(19) United States
(12) Patent Application Publication
(10) Pub. No.: US 2025/0243160 A1
(43) Pub. Date: Jul. 31, 2025

(54) INDOLE DERIVATIVES FOR TARGETING AUTOPHAGY

(71) Applicant: Automera PTE. LTD, Singapore (SG)

(72) Inventors: Shuyi Pearly NG, Singapore (SG); Mohammed Mahmood AHMED, Singapore (SG); Shawn Siang HOON, Singapore (SG); Kenrick An Fu YAP, Singapore (SG)

(21) Appl. No.: 19/035,678

(22) Filed: Jan. 23, 2025

Related U.S. Application Data
(60) Provisional application No. 63/624,642, filed on Jan. 24, 2024.

(51) Int. Cl. C07D 209/08; A61K 31/4045; A61K 31/4178; A61K 31/437; A61K 31/454; C07D 401/06; C07D 403/06; C07D 471/04; C07D 495/14

(52) U.S. Cl. CPC C07D 209/08; A61K 31/4045; A61K 31/4178; A61K 31/437; A61K 31/454; C07D 401/06; C07D 403/06; C07D 471/04; C07D 495/14

(57) ABSTRACT

Provided herein are compounds of formula (I), (II), and (III), or pharmaceutically acceptable salts, stereoisomers, or deuterated forms thereof, wherein X¹, X², Y¹, Y², Y³, R¹, R², a, b, L⁰, L¹, L², and A are defined herein. Also provided herein are pharmaceutical compositions comprising a compound of formula (I), (II), or (III), or pharmaceutically acceptable salt, a stereoisomer, or deuterated form thereof, and methods of using a compound of formula (I), (II), or (III), or pharmaceutically acceptable salt, a stereoisomer, or deuterated form thereof, e.g., in the treatment of a disease or disorder by modulating autophagic degradation and/or p62 activity.

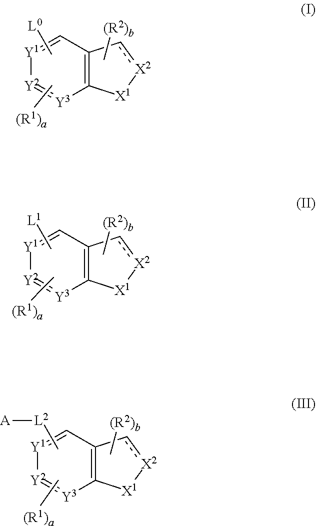

### CROSS-REFERENCE TO RELATED APPLICATIONS

This application claims priority to U.S. Provisional Patent Application No. 63/624,642, filed on Jan. 24, 2024, the content of which is hereby incorporated by reference in its entirety for all purposes.

### BACKGROUND

Autophagy is a major degradation pathway for maintaining cellular homeostasis. Autophagy eliminates unnecessary, aged, dysfunctional, or damaged intracellular components through lysosome-mediated degradation. The autophagy process can degrade bulky cellular cargoes (e.g., proteins aggregates, intracellular pathogens) that cannot be degraded by other processes, such as the ubiquitin-proteasome system (UPS).

During autophagy, cytoplasmic contents are delivered into the lysosomal system by double-membraned organelles called autophagosomes. Specifically, cytoplasmic material is sequestered into autophagosomes, which subsequently fuse with lysosomes where degradation occurs via the action of acidic lysosomal hydrolases. Autophagy helps to keep cells healthy, and dysregulation of this process can contribute to a wide range of diseases, including cancer, inflammation, neurodegeneration, and infectious diseases.

Sequestosome-1, also known as ubiquitin-binding protein p62 (SQSTM1 or p62, hereinafter “p62”), is a key autophagy receptor for targeted degradation. p62 operates as an autophagy adaptor that brings ubiquitinated substrates (e.g., damaged proteins) into contact with autophagosomes in preparation for autophagy. In addition to autophagic degradation, p62 also plays an important role in the UPS, cellular signaling, metabolism, and apoptosis.

There is a need to develop therapeutics that target p62 and modulate autophagy.

### SUMMARY

In one aspect, the present disclosure provides a compound of formula (I):

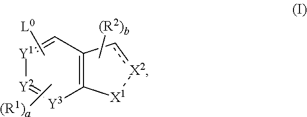

or a pharmaceutically acceptable salt, stereoisomer, or deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In another aspect, the present disclosure provides a compound of formula (II)

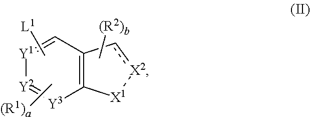

or a pharmaceutically acceptable salt, stereoisomer, or deuterated form thereof,
wherein:
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In a further aspect, the present disclosure provides a compound of formula (III)

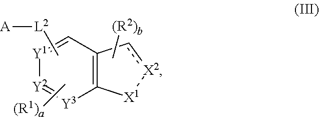

or a pharmaceutically acceptable salt, stereoisomer, or deuterated form thereof,
wherein:
   A is a ligand that binds to a protein, a protein aggregate, a protein complex, or a lipid;
   L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, arylene, heteroarylene, and heterocyclylene are each optionally and independently substituted with 1, 2, or 3 R^{Z}, wherein each R^{Z} is independently OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

### BRIEF DESCRIPTION OF THE DRAWINGS

FIG. 1A illustrates general synthetic route A for preparing compounds of the present disclosure.

FIG. 1B illustrates general synthetic route B for preparing compounds of the present disclosure.

FIG. 1C illustrates general synthetic route C for preparing compounds of the present disclosure.

FIG. 1D illustrates general synthetic route D for preparing compounds of the present disclosure.

FIG. 1E illustrates general synthetic route E for preparing compounds of the present disclosure.

FIG. 1F illustrates general synthetic route F for preparing compounds of the present disclosure.

FIG. 1G illustrates general synthetic route G for preparing compounds of the present disclosure.

FIG. 1H illustrates general synthetic route H for preparing compounds of the present disclosure.

FIG. 1I illustrates general synthetic route I for preparing compounds of the present disclosure.

FIG. 1J illustrates general synthetic route J for preparing compounds of the present disclosure.

FIG. 1K illustrates general synthetic route K for preparing compounds of the present disclosure.

FIGS. 2A-2C shows representative western blots for p62 oligomerization of compounds 31 (FIG. 2A), 34 (FIG. 2B), and 45 (FIG. 2C).

FIGS. 3A-3C shows representative Western blots for LC3 lipidation of compounds 31 (FIG. 3A), 34 (FIG. 3B), and 45 (FIG. 3C).

FIGS. 4A-4B shows representative Western blots for protein degradation by compound 97 (FIG. 4A) and compound 101 (FIG. 4B).

FIG. 5 shows representative Western blots for protein degradation by compound 107.

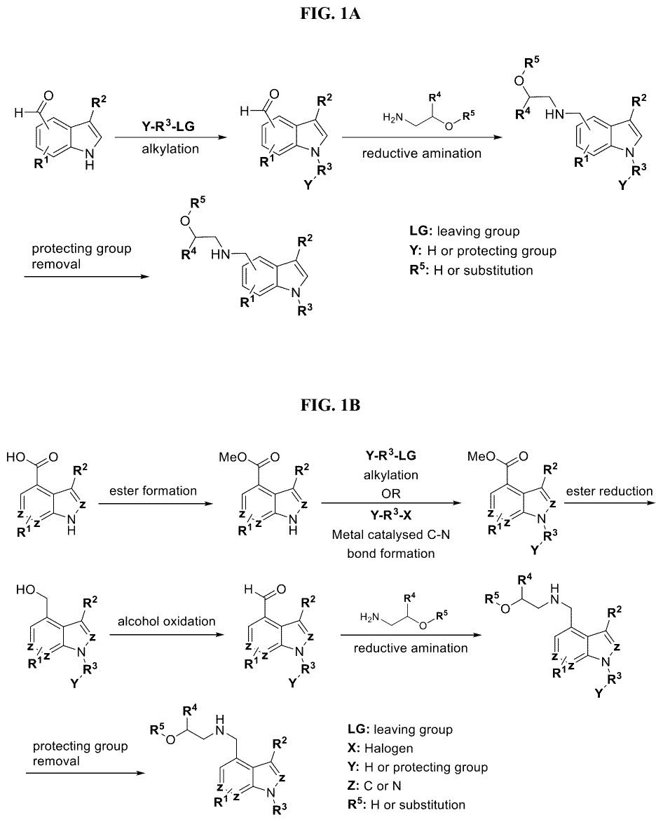

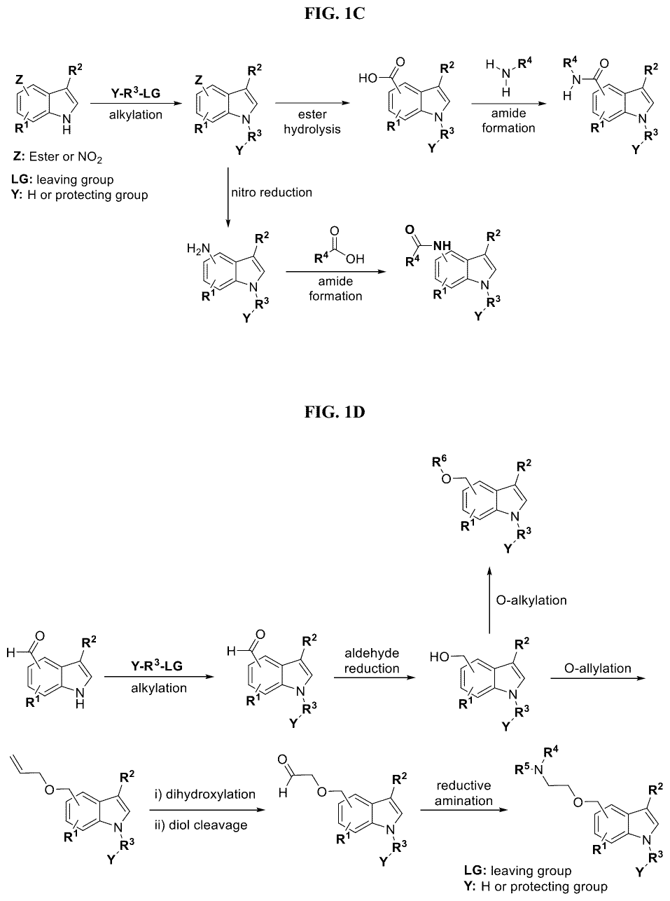

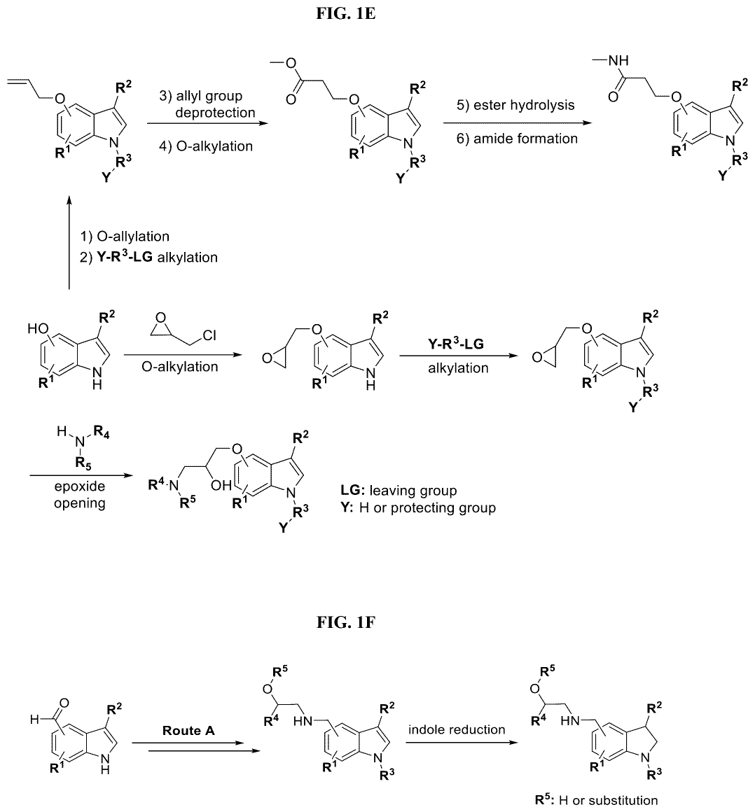

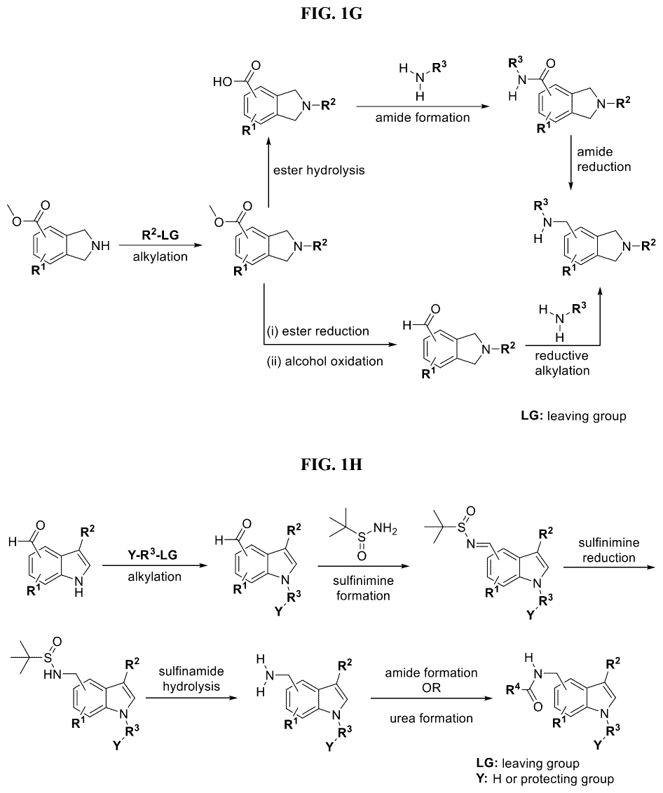

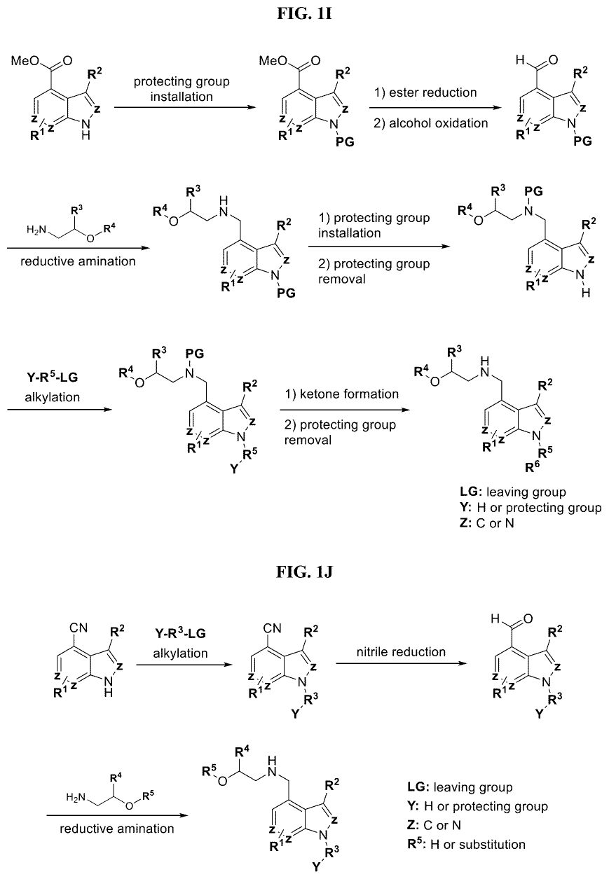

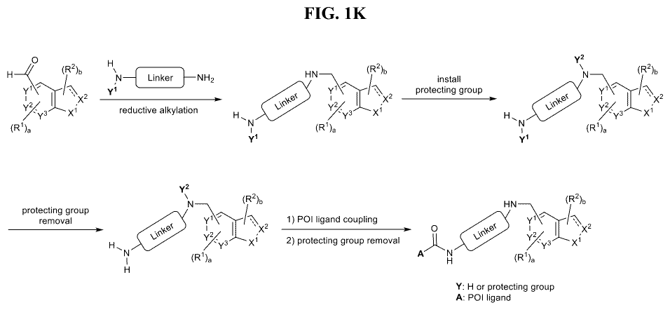

### DETAILED DESCRIPTION

Targeted Protein degradation (TPD) has emerged as a promising modality in drug discovery, typically involving heterobifunctional molecules that combine a target binder with a degradation-inducing moiety (Békés, Miklós, David R. Langley, and Craig M. Crews. 2022. “PROTAC Targeted Protein Degraders: The Past Is Prologue.” Nature Reviews. Drug Discovery 21 (3): 181-200, incorporated herein by reference in its entirety). This approach is particularly attractive for targeting proteins previously considered undruggable. However, current TPD technologies, such as PROteolysis-TArgeting Chimeras (PROTACs), are primarily limited to inducing ubiquitination of target substrates for degradation. Despite its potential, PROTAC technology faces limitations due to challenges in forming substrate-PROTAC-E3 ligase complexes, restricting its application to a limited set of targets and E3 ligases.

Autophagy is a vital cellular process that manages the degradation and recycling of cellular components (Lamark, Trond, and Terje Johansen. 2021. “Mechanisms of Selective Autophagy.” Annual Review of Cell and Developmental Biology 37 (October): 143-69; Aman, Yahyah, Tomas Schmauck-Medina, Malene Hansen, Richard I. Morimoto, Anna Katharina Simon, Ivana Bjedov, Konstantinos Palikaras, et al. 2021. “Autophagy in Healthy Aging and Disease.” Nature Aging 1 (8): 634-50; each incorporated herein by reference in their entirety). It plays a crucial role in maintaining cellular homeostasis by removing damaged or dysfunctional organelles and proteins. The process of autophagy involves the formation of autophagosomes, which engulf cellular debris and fuse with lysosomes for degradation. This mechanism is essential for cellular health, and its dysfunction is associated with various diseases, including neurodegenerative disorders, cancers, and metabolic diseases. Autophagy can be broadly classified into macroautophagy, microautophagy, and chaperone-mediated autophagy, with each type serving specific functions in the cell.

Autophagy plays a dual role in disease pathology. While it can prevent the accumulation of toxic protein aggregates and damaged organelles, its dysregulation can also contribute to disease progression. In neurodegenerative diseases such as Alzheimer's, Parkinson's, and Huntington's disease, impaired autophagy leads to the accumulation of misfolded proteins, exacerbating neurodegeneration. In cancer, autophagy can have both tumor-suppressive and tumor-promoting roles, depending on the context. Enhancing or inhibiting autophagy has been explored as a therapeutic strategy in various diseases. For instance, autophagy inducers are being investigated for their potential to clear protein aggregates in neurodegenerative diseases, while autophagy inhibitors are being explored in certain cancers (Maiuri, Maria Chiara, and Guido Kroemer. 2019. “Therapeutic Modulation of Autophagy: Which Disease Comes First?” Cell Death and Differentiation 26 (4): 680-89, incorporated herein by reference in its entirety).

p62 is a multifunctional protein that plays a significant role in autophagy, particularly in selective autophagy (Kumar, Anita V., Joslyn Mills, and Louis R. Lapierre. 2022. “Selective Autophagy Receptor P62/SQSTM1, a Pivotal Player in Stress and Aging.” Frontiers in Cell and Developmental Biology 10 (February): 793328, incorporated herein by reference in its entirety). It acts as a link between LC3 (a protein associated with autophagosomes) and ubiquitinated substrates. p62 binds to ubiquitin-tagged proteins and aggregates, delivering them to autophagosomes for degradation. It is also involved in the formation of protein aggregates known as aggresomes, which are targeted for autophagic degradation. The regulation of p62 and its interaction with other autophagy-related proteins are critical for the efficient execution of selective autophagy.

p62 is a key autophagy adaptor involved in the autophagic degradation of ubiquitinated substrates. p62 also interacts with ATG8 proteins that are found on the surface of developing autophagosomes. As such, p62 enables selective degradation by directing ubiquitinated substrates to the growing autophagosomes. Oligomerization of individual p62 units (i.e., p62 oligomer) has been shown to provide a stronger interaction with autophagosome. In addition to autophagic degradation, p62 influences other cellular pathways and is associated with pathological conditions including neurodegenerative diseases and cancer.

The N-degron pathway recognizes specific N-terminal amino acids (N-degrons) of proteins for degradation (Varshavsky, Alexander. 2019. “N-Degron and C-Degron Pathways of Protein Degradation.” Proceedings of the National Academy of Sciences of the United States of America 116 (2): 358-66, incorporated herein by reference in its entirety). The Arginylation branch of this pathway utilizes Arg, Lys, His (type 1), and Phe, Tyr, Trp, Leu, Ile (type 2) as N-degrons. Recent discoveries have shown that the Arg/N-degron pathway mediates not only ubiquitylation-dependent proteasomal clearance but also macroautophagic protein degradation. In this process, p62/SQSTM1 acts as an N-recognin, binding type-1 and type-2 N-degrons via its ZZ domain, activating p62 into an autophagy-compatible form for efficient autophagosome biogenesis (Kwon, Do Hoon, Ok Hyun Park, Leehyeon Kim, Yang Ouk Jung, Yeonkyoung Park, Hyeongseop Jeong, Jaekyung Hyun, Yoon Ki Kim, and Hyun Kyu Song. 2018. “Insights into Degradation Mechanism of N-End Rule Substrates by P62/SQSTM1 Autophagy Adapter.” Nature Communications 9 (1): 3291; Cha-Molstad, Hyunjoo, Ji Eun Yu, Zhiwei Feng, Su Hyun Lee, Jung Gi Kim, Peng Yang, Bitnara Han, et al. 2017. “P62/SQSTM1/Sequestosome-1 Is an N-Recognin of the N-End Rule Pathway Which Modulates Autophagosome Biogenesis.” Nature Communications 8 (1): 1-17; each incorporated herein by reference in their entirety).

p62 (SQSTM1) and NBR1 are key autophagy receptors that mediate the selective degradation of ubiquitinated proteins by bridging cargo to the autophagic machinery. Both proteins share several conserved domains critical to their function, including the ZZ-type zinc finger domain (ZZ domain), a PB1 domain for oligomerization, a UBA domain for binding polyubiquitinated substrates, and an LC3-interacting region (LIR) essential for autophagosome recruitment.

A defining feature of p62 and NBR1 is their ability to bind LC3, a core autophagy component embedded in the autophagosome membrane. This interaction, driven by their LIR motifs, is crucial for targeting cargo to autophagosomes for subsequent lysosomal degradation. Oligomerization through the PB1 domain promotes the clustering of cargo, enhancing both LC3 binding and autophagosome formation. The ZZ domain further stabilizes this process by indirectly facilitating interactions with ubiquitinated substrates and upstream signalling proteins involved in autophagy initiation.

Given the high degree of conservation between p62 and NBR1, the designed warheads could potentially interact with both receptors, offering a broad mechanism for modulating selective autophagy.

This disclosure presents compositions and methods for the manipulation of the intrinsic autophagic pathway for the selective degradation of pathogenic proteins. The disclosure comprises novel heterobifunctional compounds designed to engage the autophagy adaptor protein p62/SQSTM1, thereby triggering its activation and subsequent assembly of autophagosomes. These chimeric molecules are composed of a targeting ligand (also referred to as a “protein binding component” or “PBC”), which exhibits high-affinity binding to designated pathogenic proteins, conjoined via a designed, flexible linker to a p62 activating moiety (also referred to as a “warhead”). This bifunctional architecture enables the precise orchestration of p62 oligomerization and spatial localization, thereby enhancing the sequestration of the targeted proteins within nascent autophagosomes. The utility of this inventive approach lies in its capacity to harness the cell's autophagic machinery, thereby offering a therapeutic modality with broad-spectrum applicability in the attenuation of diseases characterized by aberrant protein accumulation or defective protein clearance.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

Listed below are definitions of various terms used in the specification and claims to describe the present disclosure.

Unless defined otherwise, all technical and scientific terms used in this disclosure have the same meanings as commonly understood by one of ordinary skill in the art to which this disclosure belongs.

The term “about” when immediately preceding a numerical value means a range encompassing said numerical value plus or minus an acceptable amount of variation in the art (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The terms below, as used herein, have the following meanings, unless indicated otherwise:

“Cyano” refers to the —CN radical.

“Hydroxy” or “hydroxyl” refers to the —OH radical.

“Oxo” refers to the ═O substituent.

“Halo,” “halogen” or “halide” refers to fluoro (—F), chloro (—Cl), bromo (—Br), and iodo (—I).

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 50 are included. An alkyl comprising up to 50 carbon atoms is a C₁-C₅₀ alkyl, an alkyl comprising up to 24 carbon atoms is a C₁-C₂₄ alkyl, an alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from 1 to 50 carbon atoms. Non-limiting examples of C₂-C₅₀ alkylene include ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 25 are included. An alkenyl group comprising up to 25 carbon atoms is a C₂-C₂₅ alkenyl, an alkenyl comprising up to 10 carbon atoms is a C₂-C₁₀ alkenyl, an alkenyl group comprising up to 6 carbon atoms is a C₂-C₆ alkenyl, and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkenyl group can be optionally substituted.

“Alkenylene” or “alkenylene chain” refers to a straight or branched divalent hydrocarbon chain radical, having from 2 to 25 carbon atoms, and having one or more carbon-carbon double bonds. Non-limiting examples of C₂-C₂₅ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from 2 to 25 carbon atoms, and having one or more carbon-carbon triple bonds. Each alkynyl group is attached to the rest of the molecule by a single bond. Alkynyl group comprising any number of carbon atoms from 2 to 25 are included. An alkynyl group comprising up to 25 carbon atoms is a C₂-C₂₅ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₂₅ alkynyl include ethynyl, propynyl, butynyl, pentynyl and the like. Unless stated otherwise specifically in the specification, an alkynyl group can be optionally substituted.

“Alkynylene” or “alkynylene chain” refers to a straight or branched divalent hydrocarbon chain radical, having from 2 to 25 carbon atoms, and having one or more carbon-carbon triple bonds. Non-limiting examples of C₂-C₂₅ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Hydroxyalkyl” refers to an alkyl radical, as defined above, that is substituted by one or more hydroxy groups. As used herein, the term “hydroxyalkyl” encompasses alkyls having a primary (terminal) hydroxy group, such as —CH₂OH, —CH₂CH₂OH, —CH₂CH₂CH₂OH, —CH₂CH(CH₃)CH₂OH, and —CH₂CH₂CH₂CH₂OH, those having branched (non-terminal) hydroxy groups, such as —CH(OH)CH₃, —CH₂CH(CH₃)OH, and those having both primary and branched hydroxy groups, such as —CH₂CH(OH)CH₂CH₂OH.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Aryl” refers to a hydrocarbon ring system radical comprising hydrogen, 6 to 18 carbon ring atoms and at least one aromatic ring. For purposes of this disclosure, the aryl radical can be a monocyclic, bicyclic, tricyclic or tetracyclic ring system, which can include fused, bridged, or spiro ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an arylalkyl group can be optionally substituted.

“Carbocycle,” “carbocyclyl,” or “carbocyclic ring” or refers to a ring structure, wherein the atoms which form the ring are each carbon. Carbocyclic rings can comprise from 3 to 20 carbon atoms in the ring. Carbocyclic rings include cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiro ring systems, having from three to twenty carbon atoms, e.g., having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkylene” refers to a divalent, non-aromatic, and fully saturated monocyclic or polycyclic hydrocarbon ring having 3 to 20 carbon atoms, or 3 to 8 carbon atoms. Non-limiting examples of C₃₋₈ cycloalkylene include

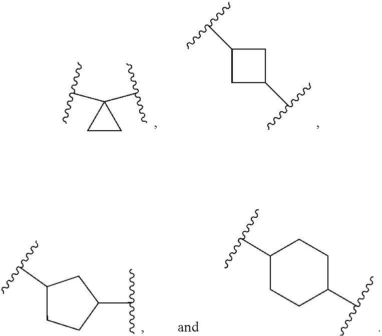 [SMILES: *C1(*)CC1 ; *C1CC(*)C1 ; *C1CCC(*)C1 ; *C1CCC(*)CC1]

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiro ring systems, having from three to twenty carbon atoms, e.g., having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiro ring systems, having from three to twenty carbon atoms, e.g., having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., difluoromethyl, trifluoromethyl, trichloromethyl, 2,2,2-trifluoroethyl, 1,2-difluoroethyl, 3-bromo-2-fluoropropyl, 1,2-dibromoethyl, and the like. The haloalkyl group of the present disclosure can be e.g., a C₁₋₁₀ haloalkyl group, a C₁₋₆ haloalkyl group, or a C₁₋₃ haloalkyl group. Unless stated otherwise specifically in the specification, a haloalkyl group can be optionally substituted.

“Heterocyclyl” “heterocyclic ring” or “heterocycle” refers to a stable 3- to 20-membered non-aromatic, saturated or partially unsaturated ring radical which consists of two to twelve carbon ring atoms and from one to six heteroatoms as ring atoms selected from nitrogen, oxygen or sulfur, at least one non-aromatic, saturated or partially unsaturated ring containing at least one heteroatom as a ring atom. Unless stated otherwise specifically in the specification, the heterocyclyl radical can be a monocyclic, bicyclic, tricyclic or tetracyclic ring system, which can include fused, bridged, or spiro ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In embodiments where “L” is heterocyclyl, the heterocyclyl radical is a diradical. Unless stated otherwise specifically in the specification, a heterocyclyl group can be optionally substituted.

“Heteroaryl” refers to a 5- to 20-membered ring system radical comprising one to thirteen carbon ring atoms, one to six heteroatoms as ring atoms selected from nitrogen, oxygen and sulfur, and at least one aromatic ring containing at least one heteroatom as a ring atom. For purposes of this disclosure, the heteroaryl radical can be a monocyclic, bicyclic, tricyclic or tetracyclic ring system, which can include fused, bridged, or spiro ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). Unless stated otherwise specifically in the specification, a heteroaryl group can be optionally substituted.

“Leaving group” refers to a functional group that can be substituted by another functional group during a chemical reaction. Exemplary leaving groups can be found in e.g., Organic Chemistry, Francis Carey, 2^{nd} edition, pages 328-331, McGraw-Hill Book Company, 1992, incorporated by reference herein. Non-limiting examples of leaving group include halogens (e.g., Cl, Br, I), methanesulfonyl (mesyl, Ms), p-toluenesulfonyl (tosyl, Ts), fluoromethanesulfonyl, difluoromethanesulfonyl, trifluoromethylsulfonyl (triflate, Tf), ethanesulfonyl, diazonium group,

“Protecting group” refers to a moiety that, when attached to a chemically reactive group in a molecule, masks or reduces chemical reactivity of the group. Protecting groups are well known in the art and include those described in detail in Protective Groups in Organic Synthesis, T. W. Greene, et al., 3^{rd} edition, John Wiley & Sons, 1999, incorporated by reference herein. Non-limiting examples of an amino protecting group (also referred to as a nitrogen protecting group) include those forming carbamates, such as tert-Butyloxycarbonyl (BOC) group, Carbobenzyloxy (Cbz) group, p-Methoxybenzyl carbonyl (Moz or MeOZ) group, Troc, 9-Fluorenylmethyloxycarbonyl (Fmoc) group, etc., those forming an amide, such as acetyl, trifluoroacetyl, benzoyl, etc., those forming a benzylic amine, such as benzyl, p-methoxybenzyl, 3,4-dimethoxybenzyl, etc., and others such as p-methoxyphenyl. Non-limiting examples of a hydroxy protecting group (also referred to as an oxygen protecting group) include those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), and those forming esters such as formate, acetate, chloroacetate, dichloroacetate, trichloroacetate, trifluoroacetate, methoxyacetate, etc.

The term “substituted” used herein means any of the above groups (i.e., alkyl, alkylene, alkenyl, alkenylene, alkynyl, alkynylene, alkoxy, alkylamino, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further includes any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

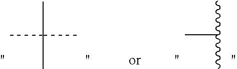

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

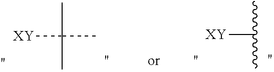

indicates that the chemical entity “XY” is bonded to another chemical entity via the point of attachment bond. Furthermore, the specific point of attachment to the non-depicted chemical entity can be specified by inference.

In this specification, unless stated otherwise, the term “pharmaceutically acceptable” is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

The term “pharmaceutically acceptable salt” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)- isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

The present disclosure is intended to encompass deuterated forms of the compounds described herein, which include isotopes of atoms occurring in the compounds. Isotopes include those atoms having the same atomic number but different mass numbers. By way of general example, and without limitation, isotopes of hydrogen include deuterium and tritium, and isotopes of carbon include ¹³C and ¹⁴C. Isotopically labeled compounds of the present disclosure can generally be prepared by conventional techniques known to those skilled in the art or by processes and methods analogous to those described herein, using an appropriate isotopically labeled reagent in place of the non-labeled reagent otherwise employed.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “derivative” refers to a chemically or biologically modified version of a chemical compound that is structurally similar to a parent compound and derivable from that parent compound. Derivatization (i.e., modification) may involve substitution of one or more moieties within the parent compound (e.g., a change in functional group). For example, when ligand A of the present disclosure is a derivative of a compound, ligand A can have a structure in which part of the structure of the compound is modified by binding to linker L². Exemplary modifications include replacement of a substituent (e.g., H, halogen, etc.) for subsequent formation of a bond via chemical process such as amidation, amination, acylation, alkylation, esterification, or dehydration.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human.

The term “treating” as used herein with regard to a patient, refers to improving at least one symptom of the patient's disorder. Treating can be improving, or at least partially ameliorating a disorder or an associated symptom of a disorder.

An “effective amount” means the amount compound or pharmaceutical formulation, that when administered to a patient for treating a state, disorder or condition is sufficient to affect such treatment.

The term “therapeutically effective” applied to dose or amount refers to that quantity of a compound or pharmaceutical formulation that is sufficient to result in a desired clinical benefit after administration to a patient in need thereof. A “therapeutically effective amount”, in some embodiments, is a dose or amount of a compound or pharmaceutical formulation that is sufficient to result in prophylaxis after administration to a patient in need thereof.

### Compounds

This disclosure presents compositions and methods for the manipulation of the intrinsic autophagic pathway for the selective degradation of pathogenic proteins. The disclosure comprises novel heterobifunctional compounds designed to engage the autophagy adaptor protein p62/SQSTM1, thereby triggering its activation and subsequent assembly of autophagosomes. These chimeric molecules are composed of a targeting ligand (also referred to as a “protein binding component” or “PBC”), which exhibits high-affinity binding to designated pathogenic proteins, conjoined via a designed, flexible linker to a p62 activating moiety (also referred to as a “warhead”). This bifunctional architecture enables the precise orchestration of p62 oligomerization and spatial localization, thereby enhancing the sequestration of the targeted proteins within nascent autophagosomes. The utility of this inventive approach lies in its capacity to harness the cell's autophagic machinery, thereby offering a therapeutic modality with broad-spectrum applicability in the attenuation of diseases characterized by aberrant protein accumulation or defective protein clearance.

In embodiments, the compounds of the present disclosure can be useful for targeted protein degradation, including for inducing targeted autophagy. In embodiments, the compounds of the present disclosure can be also useful for modulating activity of p62.

In one aspect, the disclosure provides compounds that target p62 and thereby modulating autophagy (e.g., compounds of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), and (II-G-1)). These compounds can be used on their own as therapeutics for modulating autophagy and treating diseases such as neurodegeneration and cancer. Alternatively or additionally, these compounds can be utilized in targeted protein degradation by functioning as adapter protein warheads (hereinafter “warheads”) that engage p62, and thereby bring p62 into proximity with a protein targeted for degradation. To leverage p62 for targeted protein degradation, in aspects, the disclosure provides for bifunctional compounds.

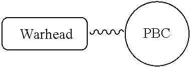

As illustrated above, these bifunctional compounds contain: (1) a first component (“warhead”) that targets and recruits an autophagy adaptor such as p62, and (2) a second component (“protein binding component” or “PBC”) that binds to a protein target to be degraded. In some embodiments, ligand A in the formulas described herein is a PBC. In embodiments, bifunctional compounds contain (3) a linker that covalently couples the warhead to the protein binding component. As such, the compounds disclosed herein can be applied for therapeutically degrading any specific targets, including, but not limited to, proteins, protein aggregates, protein complexes, lipids, lipid droplets, or pathogens (e.g., viruses) within the cell. Additional autophagy adaptor proteins include, but are not limited to, LC3, Optineurin, TAX1BP1, NBR1, NDP52, NUFIP1, WDFY3, RETREG1, Nix, and TOLLIP.

In some embodiments, ligand A and L², which will be detailed further below, function as the protein binding component (PBC) and linker, respectively, of the bifunctional compounds of formula (X-III), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1) described herein.

In embodiments, the present disclosure provides a compound of formula (X-I):

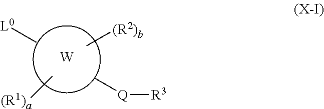

or a pharmaceutically acceptable salt, stereoisomer, or deuterated form thereof,
wherein:
   ring W is 6,5-fused heteroaryl or 6,5-fused heterocycle ring, wherein each ring W contains 1, 2, or 3 heteroatoms selected from N, O, or S, and at least 1 of the heteroatoms is N or O;
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle;
   each R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (X-I):

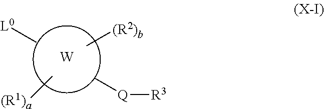

or a pharmaceutically acceptable salt, stereoisomer, or deuterated form thereof,
wherein:
   ring W is 6,5-fused heteroaryl or 6,5-fused heterocycle ring, wherein each ring W contains 1, 2, or 3 heteroatoms selected from N, O, or S, and at least 1 of the heteroatoms is N or O;
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle;
   each R^{B} is C₁₋₆ hydroxyalkyl, C₁₋₆ alkoxy, C₁₋₆ alkylene-NH₂, or C₁₋₆ alkylene-SH;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I):

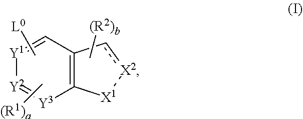

or a pharmaceutically acceptable salt, stereoisomer, or deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle;
   each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I):

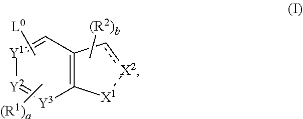

or a pharmaceutically acceptable salt, stereoisomer, or deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle;
   each R^{B} is C₁₋₆ hydroxyalkyl, C₁₋₆ alkoxy, C₁₋₆ alkylene-NH₂, or C₁₋₆ alkylene-SH;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-A):

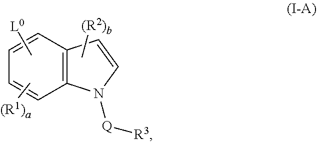

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-A):

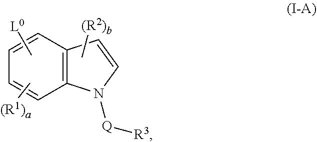

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ hydroxyalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-A-1), (I-A-2), or (I-A-3):

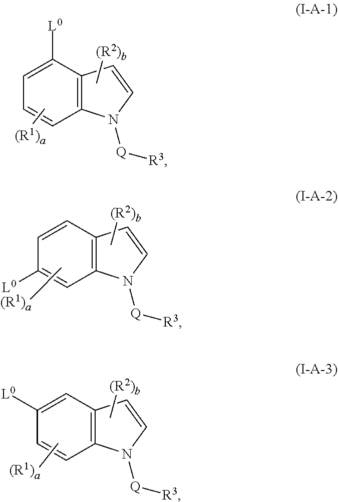

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of of formula (I-A-1), (I-A-2), or (I-A-3):

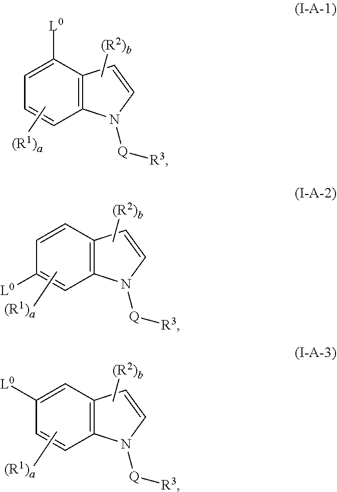

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ hydroxyalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-B), (I-C), or (I-D):

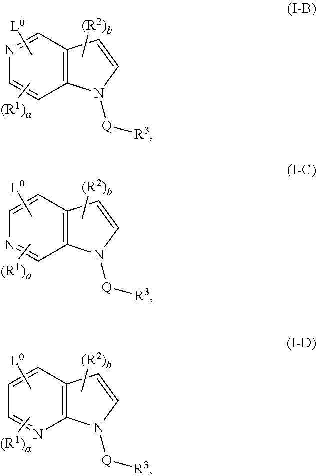

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-B), (I-C), or (I-D):

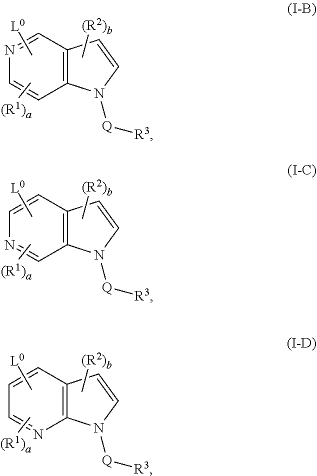

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
each R¹ is independently C₁₋₆ alkyl, C₃-C₈ cycloalkyl, or halogen;
each R² is independently C₁₋₆ alkyl, C₃-C₈ cycloalkyl, or halogen, or two R² form an oxo;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle,
each R^{B} is independently C₁₋₆ hydroxyalkyl, C₁₋₆ alkoxy, C₁₋₆ alkylene-NH₂, or C₁₋₆ alkylene-SH,
R^{X} is H, C₁₋₆ alkyl, C₁₋₆ haloalkyl, or C₁₋₆ hydroxyalkyl;
R^{Y} is C₃₋₈ cycloalkyl, C₃₋₈ hydroxycycloalkyl, aryl, or heterocycle;
a is an integer of 0-3; and
b is an integer of 0-2.

In embodiments, the present disclosure provides a compound of formula (I-B-1), (I-C-1), or (I-D-1):

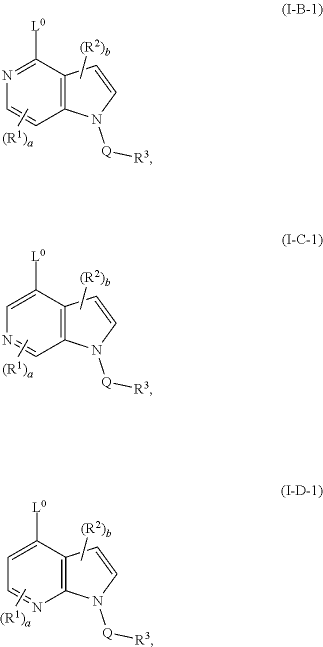

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-B-1), (I-C-1), or (I-D-1):

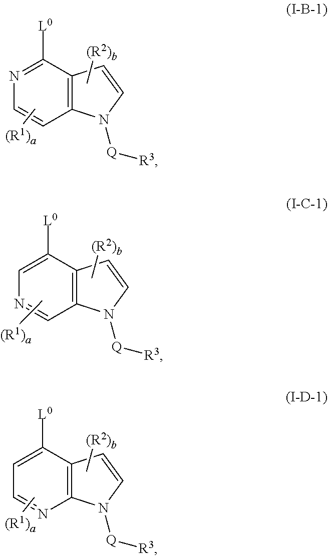

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ hydroxyalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-E), (I-F), or (I-G):

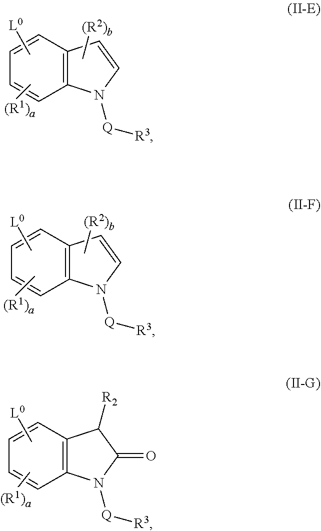

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-E), (I-F), or (I-G):

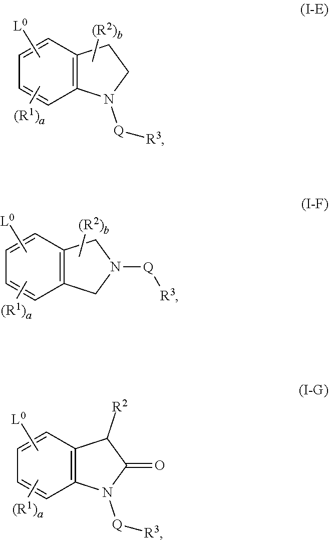

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ hydroxyalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-E-1), (I-F-1), or (I-G-1):

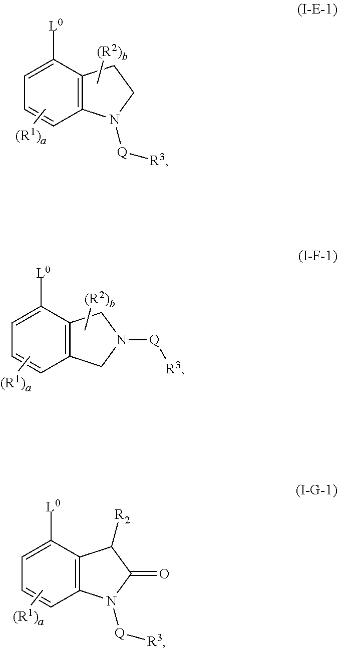

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-E-1), (I-F-1), or (I-G-1):

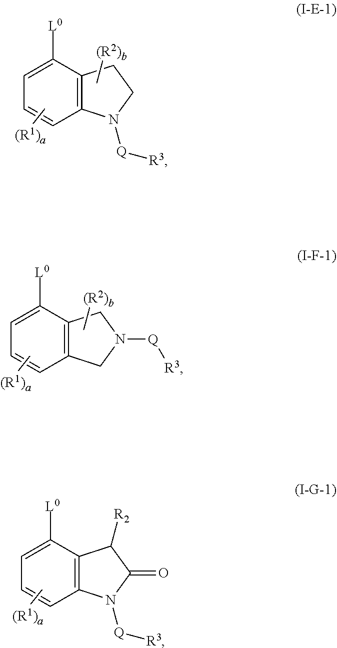

or a pharmaceutically acceptable salt, a stereoisomer, or a deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with OH or halogen,
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle,
   each R^{B} is independently C₁₋₆ hydroxyalkyl, C₁₋₆ alkoxy, C₁₋₆ alkylene-NH₂, or C₁₋₆ alkylene-SH,
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

### L⁰

In embodiments of the compound of formula (X-I), (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted. In embodiments of the compound of formula (X-I), (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted. In embodiments, the alkylene is optionally substituted with —OH, halogen, C₁₋₆ hydroxyalkyl, C₁₋₆ haloalkyl, C₃₋₈ cycloalkyl, C₁₋₆ alkoxy, —NH₂, NH(C₁-C₆ alkyl), N(C₁-C₆ alkyl)₂, S(C₁-C₆ alkyl), —SO(C₁₋₆ alkyl), —SO₂(C₁₋₆ alkyl), —SO₂N(C₁₋₆ alkyl), —SO₂NH₂, —SO₂NH(C₁-C₆ alkyl), —SO₂N(C₁-C₆ alkyl)₂, carbocycle, heterocycle, aryl, or heteroaryl. In embodiments, the alkylene is optionally substituted with —OH or halogen. In embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In embodiments, each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In embodiments, each R^{B} is independently C₁₋₆ hydroxyalkyl, C₁₋₆ alkoxy, C₁₋₆ alkylene-NH₂, or C₁₋₆ alkylene-SH.

In embodiments of the compound of formula (X-I), (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —(C₁₋₆ alkylene)-N(H)(C₁₋₆ hydroxyalkyl), —(C₁₋₆ alkylene)-N(C₁₋₆ alkyl)(C₁₋₆ hydroxyalkyl), —(C₁₋₆ alkylene)-N(H)(C₁₋₆ alkoxy), —(C₁₋₆ alkylene)-N(C₁₋₆ alkyl)(C₁₋₆ alkoxy), —(C₁₋₆ alkylene)-NH(C₁₋₆ alkylene)(C₁₋₆ alkoxy), —C(O)N(H)(C₁₋₆ hydroxyalkyl), —(C₁₋₆ alkylene)-N(H)C(O)(C₁₋₆ alkoxy), —(C₁₋₆ alkylene)-N(C₁₋₆ alkyl)C(O)(C₁₋₆ alkoxy), —(C₁₋₆ alkylene)-N(H)C(O)N(H)(C₁₋₆ alkoxy), —(C₁₋₆ alkylene)-N(C₁₋₆ alkyl)C(O)N(C₁₋₆ alkyl)(C₁₋₆ alkoxy), —(C₃₋₈ cycloalkylene)N(H)(C₁₋₆ hydroxyalkyl), —(C₃₋₈ cycloalkylene)N(C₁₋₆ alkyl)(C₁₋₆ hydroxyalkyl), —O—(C₁₋₆ alkylene)-N(H)(C₁₋₆ hydroxyalkyl), —O—(C₁₋₆ alkylene)-N(C₁₋₆ alkyl)(C₁₋₆ hydroxyalkyl), —N(H)(C₁₋₆ hydroxyalkyl), —N(H)C(O)N(H)(C₁₋₆ hydroxyalkyl), —N(H)C(O)N(H)(C₁₋₆ alkylene)-(C₁₋₆ alkoxy), —(C₁₋₆ alkylene)-O—(C₁₋₆ alkylene)C(O)N(H)(C₁₋₆ alkyl), —(C₁₋₆ alkylene)-O—(C₁₋₆ alkylene)N(H)C(O)(C₁₋₆ alkyl), —O—(C₁₋₆ alkylene)C(O)N(H)(C₁₋₆ alkyl), —N(C₁₋₆ alkyl)(C₁₋₆ hydroxyalkyl), —N(H)C(O)(C₁₋₆ hydroxyalkyl), —N(C₁₋₆ alkyl)C(O)(C₁₋₆ hydroxyalkyl), —(C₁₋₆ alkylene)-heterocyclylene-(C₁₋₆ alkoxy), —(C₁₋₆ alkylene)-O—(C₁₋₆ alkylene)-N(H)(C₁₋₆ alkyl), or —(C₁₋₆ alkylene)-O—(C₁₋₆ alkylene)-N(C₁₋₆ alkyl)(C₁₋₆ alkyl), wherein the alkylene or alkyl is optionally substituted. In embodiments of the compound of formula (X-I), (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —(C₁₋₆ alkylene)-N(H)(C₁₋₆ hydroxyalkyl), —(C₁₋₆ alkylene)-N(C₁₋₆ alkyl)(C₁₋₆ hydroxyalkyl), —(C₁₋₆ alkylene)-N(H)(C₁₋₆ alkoxy), —(C₁₋₆ alkylene)-N(C₁₋₆ alkyl)(C₁₋₆ alkoxy), —(C₁₋₆ alkylene)-N(H)C(O)(C₁₋₆ alkoxy), —(C₁₋₆ alkylene)-N(C₁₋₆ alkyl)C(O)(C₁₋₆ alkoxy), —(C₁₋₆ alkylene)-N(H)C(O)N(H)(C₁₋₆ alkoxy), —(C₁₋₆ alkylene)-N(C₁₋₆ alkyl)C(O)N(C₁₋₆ alkyl)(C₁₋₆ alkoxy), —(C₃₋₈ cycloalkylene)N(H)(C₁₋₆ hydroxyalkyl), —(C₃₋₈ cycloalkylene)N(C₁₋₆ alkyl)(C₁₋₆ hydroxyalkyl), —O—(C₁₋₆ alkylene)-N(H)(C₁₋₆ hydroxyalkyl), —O—(C₁₋₆ alkylene)-N(C₁₋₆ alkyl)(C₁₋₆ hydroxyalkyl), —N(H)(C₁₋₆ hydroxyalkyl), —N(C₁₋₆ alkyl)(C₁₋₆ hydroxyalkyl), —N(H)C(O)(C₁₋₆ hydroxyalkyl), —N(C₁₋₆ alkyl)C(O)(C₁₋₆ hydroxyalkyl), wherein the alkylene or alkyl is optionally substituted. In embodiments, the alkylene or alkyl is optionally substituted with —OH, halogen, C₁₋₆ alkyl, C₁₋₆ hydroxyalkyl, C₁₋₆ haloalkyl, C₃₋₈ cycloalkyl, C₁₋₆ alkoxy, —NH₂, NR^{A}R^{B}, S(C₁-C₆ alkyl), —SO(C₁-6 alkyl), —SO₂(C₁₋₆ alkyl), —SO₂N(C₁₋₆ alkyl), —SO₂NR^{A}R^{B}, carbocycle, heterocycle, aryl, or heteroaryl. In some embodiments, the alkylene or alkyl is optionally substituted with —OH or halogen.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-NR^{A}R^{B}. In some embodiments, L⁰ is —C₁₋₃ alkylene-NR^{A}R^{B}. In some embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In some embodiments, each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, each R^{B} is independently C₁₋₆ hydroxyalkyl, C₁₋₆ alkoxy, C₁₋₆ alkylene-NH₂, or C₁₋₆ alkylene-SH. In some embodiments, R^{A} is H, and R^{B} is C₁₋₆ hydroxyalkyl or C₃₋₆ hydroxycycloalkyl. In some embodiments, R^{B} is —CH₂CH₂OH, —CH₂CH(CH₃)OH,

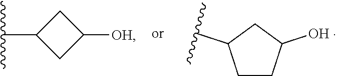 [SMILES: *C1CC(O)C1 ; "or" ; *C1CCC(O)C1 ; "."]

In some embodiments, L⁰ is —CH₂NHCH₂CH₂OH, —CHCH₃NHCH₂CH₂OH, —CH₂NHCH₂CH(CH₃)OH,

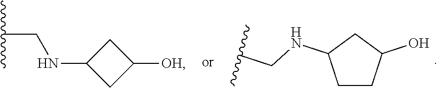 [SMILES: *CNC1CC(O)C1 ; "or" ; *CNC1CCC(O)C1 ; "."]

In some embodiments, L⁰ is —CH₂NHCH₂CH₂OH.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-NR^{A}—C₁₋₆ alkylene-R^{B}. In some embodiments, each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{B} is C₁₋₆ alkoxy and L⁰ is —(C₁₋₆ alkylene)-NR^{A}—(C₁₋₆ alkylene)-(C₁₋₆ alkoxy). In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{A} is H. In some embodiments, L⁰ is —(C₁₋₆ alkylene)-NH(C₁₋₆ alkylene)(C₁₋₆ alkoxy), In some embodiments, L⁰ is —(C₁₋₃ alkylene)-NH—(C₁₋₃ alkylene)-(C₁₋₄ alkoxy). In some embodiments, L⁰ is —CH₂NHCH₂CH₂OCH(CH₃)₂ or —CH₂NHCH₂CH₂OCH₃.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —(C₁₋₆ alkylene)-NR^{A}—(C₁₋₆ alkylene)-(C₁₋₆ alkoxy). In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{A} is H. In some embodiments, L⁰ is —(C₁₋₃ alkylene)-NH—(C₁₋₃ alkylene)-(C₂₋₄ alkoxy). In some embodiments, L⁰ is —CH₂NHCH₂CH₂OCH(CH₃)₂.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —(C₁₋₆ alkylene)-O—(C₁₋₆ alkylene)-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, L⁰ is —(C₁₋₃ alkylene)-O—(C₁₋₃ alkylene)-NHR^{B}. In some embodiments, L⁰ is —(C₁₋₆ alkylene)-O—(C₁₋₆ alkylene)-N(H)(C₁₋₆ alkyl). In some embodiments, R^{A} is C₁₋₆ alkyl. In some embodiments, L⁰ is —(C₁₋₆ alkylene)-O—(C₁₋₆ alkylene)-N(C₁₋₆ alkyl)(C₁₋₆ alkyl). In some embodiments, R^{B} is C₁₋₃ alkyl. In some embodiments, R^{B} is —CH₃ or —CH(CH₃)₂. In some embodiments, L⁰ is —CH₂OCH₂CH₂NHCH₃ or —CH₂OCH₂CH₂NHCH(CH₃)₂. In some embodiments, R^{A} is C₁₋₃ alkyl. In some embodiments, R^{B} is C₁₋₃ alkyl. In some embodiments, R^{A} and R^{B} are —CH₃. In some embodiments, L⁰ is —CH₂OCH₂CH₂N(CH₃)₂.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —(C₁₋₆ alkylene)-O—(C₁₋₆ alkylene)-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, L⁰ is —(C₁₋₃ alkylene)-O—(C₁₋₃ alkylene)-NHR^{B}. In some embodiments, R^{B} is —CH₃ or —CH(CH₃)₂. In some embodiments, L⁰ is —CH₂OCH₂CH₂NHCH₃ or —CH₂OCH₂CH₂NHCH(CH₃)₂.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —(C₁₋₆ alkylene)-O—(C₁₋₆ alkylene)-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, L⁰ is —(C₁₋₃ alkylene)-O—(C₁₋₃ alkylene)-NHR^{B}. In some embodiments, R^{B} is —CH₃ or —CH(CH₃)₂. In some embodiments, L⁰ is —CH₂OCH₂CH₂NHCH₃ or —CH₂OCH₂CH₂NHCH(CH₃)₂.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C(O)NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, R^{B} is C₁₋₆ hydroxyalkyl. In some embodiments, L⁰ is —C(O)N(H)(C₁₋₆ hydroxyalkyl), In some embodiments, L⁰ is —C(O)NHCH₂CH₂OH.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is NR^{A}C(O)R^{B} In some embodiments, R^{B} is C₁₋₆ alkyl, C₁₋₆ hydroxyalkyl, C₃₋₈ cycloalkyl, C₁₋₆ alkoxy, C₁₋₆ alkylene-NH₂, or C₁₋₆ alkylene-SH. In some embodiments, R^{B} is C₁₋₆ hydroxyalkyl, and L⁰ is —NR^{A}C(O)C₁₋₆ hydroxyalkyl. In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —NR^{A}C(O)C₁₋₆ hydroxyalkyl. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{A} is H. In some embodiments, La is —NHC(O)C₁₋₃ hydroxyalkyl. In some embodiments, L⁰ is —NHC(O)CH₂CH₂OH or —NHC(O)CH₂OH.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-NR^{A}C(O)NR^{A}R^{B}. In some embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In some embodiments, at least one R^{A} is H. In some embodiments, L⁰ is —C₁₋₃ alkylene-NHC(O)NHR^{B}. In some embodiments, R^{B} is C₁₋₆ alkyl, C₁₋₆ hydroxyalkyl, C₃₋₈ cycloalkyl, C₁₋₆ alkoxy, C₁₋₆ alkylene-NH₂, or C₁₋₆ alkylene-SH. In some embodiments, R^{B} is C₁₋₆ hydroxyalkyl. In some embodiments, L⁰ is —CH₂NHC(O)NHCH₂CH₂OH.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-NR^{A}C(O)NR^{A}R^{B}. In some embodiments, L⁰ is —C₁₋₃ alkylene-NHC(O)NR^{B}. In some embodiments, L⁰ is —CH₂NHC(O)NHCH₂CH₂OH.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₃₋₈ cycloalkylene-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, L⁰ is —C₃₋₅ cycloalkylene-NHR^{B}. In some embodiments, R^{B} is —CH₂CH₂OH. In some embodiments, L⁰ is

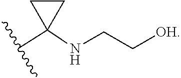 [SMILES: *C1(NCCO)CC1]

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —NR^{A}C(O)NR^{A}R^{B}. In some embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{B} is C₁₋₆ hydroxyalkyl, C₁₋₆ alkoxy, C₁₋₆ alkylene-NH₂, or C₁₋₆ alkylene-SH. In some embodiments, each R^{A} is H. In some embodiments, L⁰ is —NHC(O)NHR^{B}. In some embodiments, L⁰ is —N(H)C(O)N(H)(C₁₋₆ hydroxyalkyl). In some embodiments, R^{B} is —CH₂CH₂OH. In some embodiments, L⁰ is —NHC(O)NHCH₂CH₂OH.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —O—C₁₋₆ hydroxyalkylene-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, L⁰ is —O—(C₂₋₅ hydroxyalkylene)-NR^{B}. In some embodiments, R^{B} is C₁₋₃ alkyl. In some embodiments, R^{B} is —CH(CH₃)₂. In some embodiments, L⁰ is —OCH₂CH(OH)CH₂NHCH(CH₃)₂.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —O—C₁₋₆ hydroxyalkylene-NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, L⁰ is —O—(C₂₋₅ hydroxyalkylene)-NHR^{B}. In some embodiments, R^{B} is —CH(CH₃)₂. In some embodiments, L⁰ is —OCH₂CH(OH)CH₂NHCH(CH₃)₂.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —O—C₁₋₆ alkylene-C(O)NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, L⁰ is —O—C₁₋₃ alkylene-C(O)NHR^{B}. In some embodiments, L⁰ is —O—(C₁₋₆ alkylene)C(O)N(H)(C₁₋₆ alkyl). In some embodiments, R^{B} is C₁₋₃ alkyl. In some embodiments, R^{B} is —CH₃. In some embodiments, L⁰ is —OCH₂CH₂C(O)NHCH₃.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —O—C₁₋₆ alkylene-C(O)NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ hydroxyalkyl, C₁₋₆ alkoxy, C₁₋₆ alkylene-NH₂, or C₁₋₆ alkylene-SH. In some embodiments, R^{A} is H. In some embodiments, L⁰ is —O—C₁₋₃ alkylene-C(O)NHR^{B}. In some embodiments, R^{B} is —CH₃. In some embodiments, L⁰ is —OCH₂CH₂C(O)NHCH₃.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-O—C₁₋₆ alkylene-C(O)NR^{A}R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, R^{B} is C₁₋₆ alkyl. In some embodiments, La is —(C₁₋₆ alkylene)-O—(C₁₋₆ alkylene)C(O)N(H)(C₁₋₆ alkyl). In some embodiments, L⁰ is —C₁₋₃ alkylene-O—C₁₋₃ alkylene-C(O)NHR^{B}. In some embodiments, R^{B} is C₁₋₃ alkyl. In some embodiments, R^{B} is —CH₃. In some embodiments, L⁰ is —CH₂OCH₂CH₂C(O)NHCH₃.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-O—C₁₋₆ alkylene-NR^{A}C(O)—R^{B}. In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{B} is C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH. In some embodiments, R^{A} is H. In some embodiments, R^{B} is C₁₋₆ alkyl. In some embodiments, La is —(C₁₋₆ alkylene)-O—(C₁₋₆ alkylene)N(H)C(O)(C₁₋₆ alkyl). In some embodiments, L⁰ is —C₁₋₃ alkylene-O—C₁₋₃ alkylene-NHC(O)—R^{B}. In some embodiments, R^{B} is C₁₋₃ alkyl. In some embodiments, R^{B} is —CH₃. In some embodiments, L⁰ is —CH₂OCH₂CH₂NHC(O)CH₃.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-NR^{A}C(O)—(C₁₋₆ hydroxyalkyl). In some embodiments, R^{A} is H, C₁₋₆ alkyl, C₁₋₆ haloalkyl, C₃₋₈ cycloalkyl, C₃₋₈ hydroxycycloalkyl, aryl, or heterocycle. In some embodiments, R^{A} is H. In some embodiments, L⁰ is —(C₁₋₃ alkylene)-NHC(O)—(C₁₋₃ hydroxyalkyl). In some embodiments, L⁰ is —CH₂NHC(O)CH₂OH, or —CH₂NHC(O)CH₂CH₂OH.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —NR^{A}C(O)NR^{A}—C₁₋₆ alkylene-C₁₋₆ alkoxy. In some embodiments, each R^{A} is independently H, C₁₋₆ alkyl, C₁₋₆ haloalkyl, C₃₋₈ cycloalkyl, C₃₋₈ hydroxycycloalkyl, aryl, or heterocycle. In some embodiments, each R^{A} is H. In some embodiments, L⁰ is —N(H)C(O)N(H)(C₁₋₆ alkylene)-(C₁₋₆ alkoxy). In some embodiments, L⁰ is —NHC(O)NH—(C₁₋₃ alkylene)-(C₁₋₃ alkoxy). In some embodiments, L⁰ is —NHC(O)NHCH₂CH₂OCH₃.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —C₁₋₆ alkylene-heterocyclylene-O—C₁₋₆ alkyl. In some embodiments, L⁰ is —C₁₋₃ alkylene-heterocyclylene-O—C₁₋₃ alkyl. In some embodiments, the heterocyclylene is 3-8 membered heterocycle containing 1 or 2 heteroatoms selected from N, O, or S. In some embodiments, the heterocyclylene is R³ is 4-6 membered heterocycle containing 1 or 2 heteroatoms selected from N, or O. In some embodiments, the heterocycle is optionally substituted. In some embodiments, the heterocyclylene is

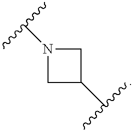 [SMILES: *C1CN(*)C1]

In some embodiments, L⁰ is

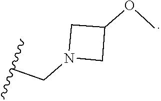 [SMILES: *CN1CC(OC)C1]

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is
—CH₂NHCH₂CH₂OH,
—CHCH₃NHCH₂CH₂OH,
—CH₂NHCH₂CH(CH₃)OH,

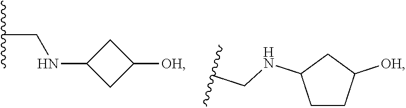 [SMILES: *CNC1CC(O)C1 ; *CNC1CCC(O)C1]

—CH₂NHCH₂CH₂OCH₃,
—CH₂NHCH₂CH₂OCH(CH₃)₂,
—CH₂OCH₂CH₂NHCH₃,
—CH₂OCH₂CH₂NHCH(CH₃)₂,
—CH₂OCH₂CH₂N(CH₃)₂,
—C(O)NHCH₂CH₂OH,
—NHC(O)CH₂CH₂OH,
—NHC(O)CH₂OH,
—CH₂NHC(O)NHCH₂CH₂OH,

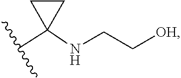 [SMILES: *C1(NCCO)CC1]

—NHC(O)NHCH₂CH₂OH,
—OCH₂CH(OH)CH₂NHCH(CH₃)₂,
—OCH₂CH₂C(O)NHCH₃,
—CH₂OCH₂CH₂C(O)NHCH₃,
—CH₂OCH₂CH₂NHC(O)CH₃,
—CH₂NHC(O)CH₂OH,
—CH₂NHC(O)CH₂CH₂OH,
—NHC(O)NHCH₂CH₂OCH₃, or

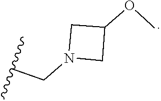 [SMILES: *CN1CC(OC)C1]

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is
—CH₂NHCH₂CH₂OH,
—CH₂NHCH₂CH(CH₃)OH,

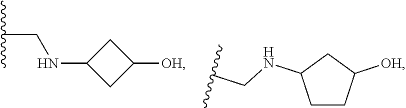 [SMILES: *CNC1CC(O)C1 ; *CNC1CCC(O)C1]

—CH₂NHCH₂CH₂OCH(CH₃)₂,
—CH₂OCH₂CH₂NHCH₃,
—CH₂OCH₂CH₂NHCH(CH₃)₂,
—C(O)NHCH₂CH₂OH,
—NHC(O)CH₂CH₂OH,
—NHC(O)CH₂OH,
—CH₂NHC(O)NHCH₂CH₂OH,

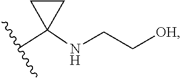 [SMILES: *C1(NCCO)CC1]

—NHC(O)NHCH₂CH₂OH,
—OCH₂CH(OH)CH₂NHCH(CH₃)₂,
—OCH₂CH₂C(O)NHCH₃,
—CH₂NHC(O)CH₂OH,
—CH₂NHC(O)CH₂CH₂OH, or
—NHC(O)NHCH₂CH₂OCH₃.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —CH₂NHCH₂CH₂OH, —CH₂NHCH₂CH(CH₃)OH, —CH₂OCH₂CH₂NHCH₃, or —OCH₂CH(OH)CH₂NHCH(CH₃)₂.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), or (I-G-1), or a pharmaceutically acceptable salt, stereoisomer, or deuterated form thereof, L⁰ is —CH₂NHCH₂CH₂OH.

In embodiments, the present disclosure provides a compound of formula (I-A-1-a), (I-A-2-a), or (I-A-3-a):

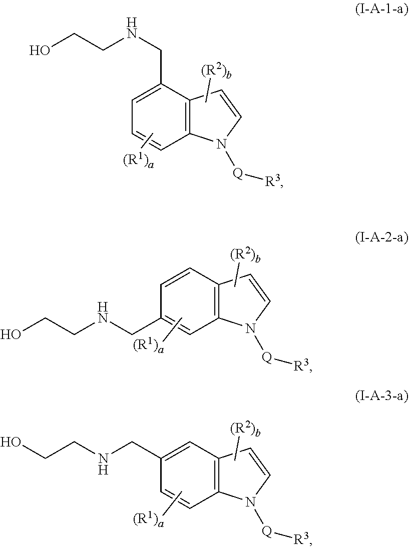

or a pharmaceutically acceptable salt, a stereoisomer, or a deuterated form thereof,
wherein:
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-B-1-a), (I-C-1-a), or (I-D-1-a):

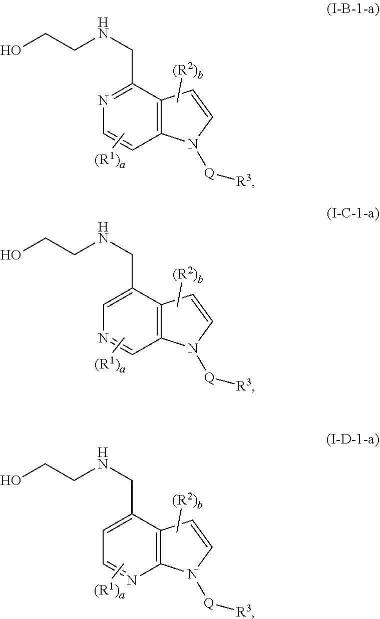

or a pharmaceutically acceptable salt, a stereoisomer, or a deuterated form thereof,
wherein:
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (I-E-1-a), (I-F-1-a), or (I-G-1-a):

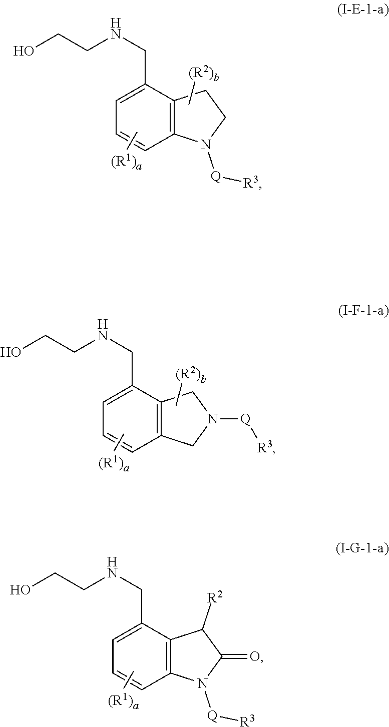

or a pharmaceutically acceptable salt, a stereoisomer, or a deuterated form thereof,
wherein:
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), or (I-G-1-a), or a pharmaceutically acceptable salt, stereoisomer, or deuterated form thereof, at least one H in L⁰ is replaced by conjugate comprising a ligand that binds to a protein, a protein aggregate, a protein complex, or a lipid.

In embodiments, the present disclosure provides for components in which a linker is covalently attached to the compounds of (X-I), (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), or (I-G-1-a). The linker (e.g., L¹ disclosed herein) may be any moiety that is capable of covalently binding to the warhead and to the protein binding component (PBC). In embodiments, the present disclosure provides a compound of formula (X-II):

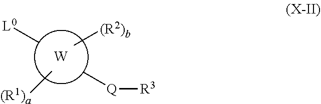

or a pharmaceutically acceptable salt, stereoisomer, or deuterated form thereof,
wherein:
   ring W is 6,5-fused heteroaryl or 6,5-fused heterocycle ring, wherein each ring W contains 1, 2, or 3 heteroatoms selected from N, O, or S, and at least 1 of the heteroatoms is N or O;
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (II)

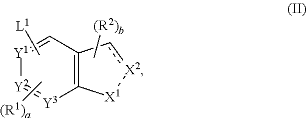

or a pharmaceutically acceptable salt, stereoisomer, or deuterated form thereof,
wherein:
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (II-A):

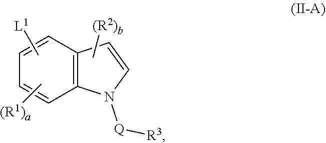

or a pharmaceutically acceptable salt, a stereoisomer, or a deuterated form thereof,
wherein:
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (II-A-1), (II-A-2), or (II-A-3):

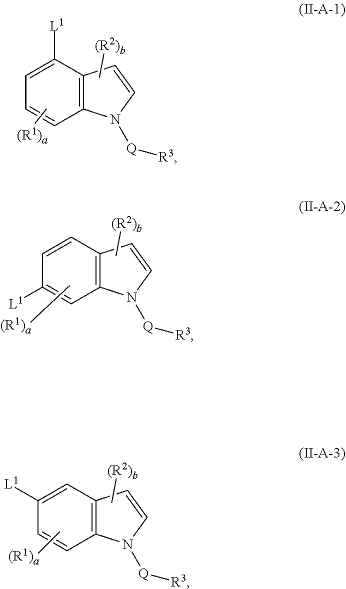

or a pharmaceutically acceptable salt, a stereoisomer, or a deuterated form thereof,
wherein:
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (II-B), (II-C), or (II-D):

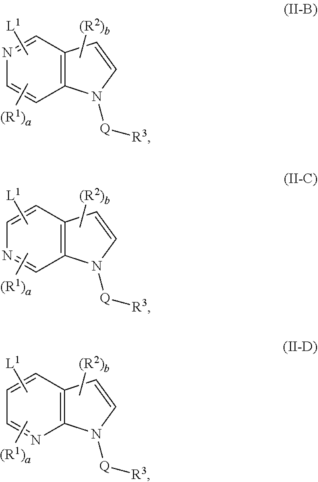

or a pharmaceutically acceptable salt, a stereoisomer, or a deuterated form thereof,
wherein:
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (II-B-1), (II-C-1), or (II-D-1):

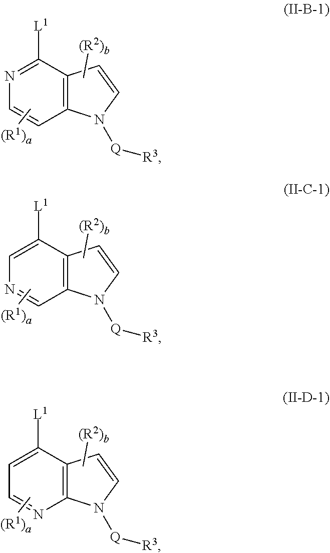

or a pharmaceutically acceptable salt, a stereoisomer, or a deuterated form thereof,
wherein:
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (II-E), (II-F), or (II-G):

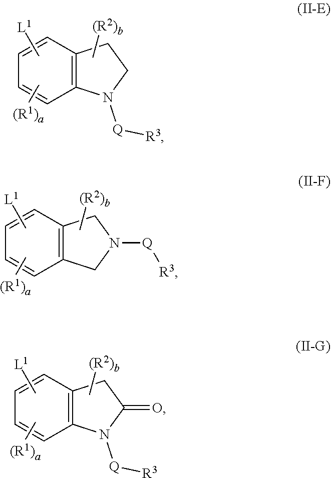

or a pharmaceutically acceptable salt, a stereoisomer, or a deuterated form thereof,
wherein:
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (II-E-1), (II-F-1), or (II-G-1):

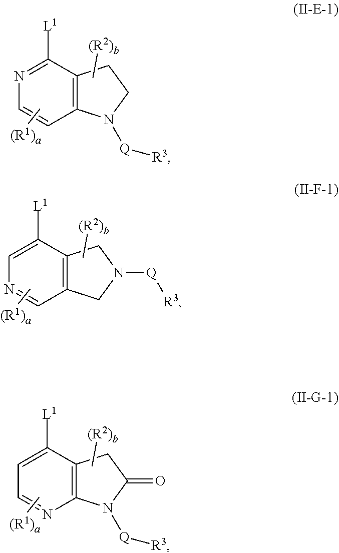

or a pharmaceutically acceptable salt, a stereoisomer, or a deuterated form thereof,
wherein:
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

### L¹

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally substituted. In embodiments, the alkylene, alkenylene, alkynylene, alkyl, arylene, heteroarylene, and heterocyclylene are each optionally substituted with 1, 2, or 3 R^{Z}, wherein each R^{Z} is independently —OH, C₁₋₆ alkyl, or halogen. In embodiments, each R⁵ is independently a bond, a leaving group, a protecting group, H, alkynyl, aryl, or heteroaryl.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —N(H)C(O)—, —N(C₁-C₆ alkyl)C(O)—, —N(C₃-C₈ cycloalkyl)C(O)—, —C(O)N(H)—, —C(O)N(C₁-C₆ alkyl)-, —C(O)N(C₃-C₈ cycloalkyl)-, arylene, heteroarylene, heterocyclylene, or C₃-C₈ cycloalkylene, wherein each arylene or heteroarylene is optionally and independently substituted. In some embodiments, each arylene or heteroarylene is optionally and independently substituted with 1 or 2 R^{Z}.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—C≡CH. In some embodiments, L¹ is —(C₁₋₃ alkylene)-NH—(CH₂CH₂O)ₘ—(CH₂)ₙ—C≡CH. In some embodiments, the alkylene is optionally substituted with 1 or 2 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-12. In some embodiments, n is an integer of 0-8. In some embodiments, L¹ is —CH₂NH—(CH₂CH₂O)—(CH₂)₂—C≡CH, —CH₂NH—(CH₂CH₂O)—(CH₂)₅—C≡CH, —CH₂NH—(CH₂CH₂O)—(CH₂)₆—C≡CH, —CH₂NH—(CH₂CH₂O)—(CH₂)₈—C≡CH, —CH₂NH—(CH₂CH₂O)₂—(CH₂)—C≡CH, —CH₂NH—[CH₂CH₂O]₂—[CH₂]₂—C≡CH, —CH₂NH—(CH₂CH₂O)₃—(CH₂)—C≡CH, or —CH₂NH—[CH₂CH₂O]₃—[CH₂]₂—C≡CH.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₃ alkylene)-NH—[CH₂CH₂O]ₘ—[CH₂]ₙ—C≡CH. In some embodiments, the alkylene is optionally substituted with 1 or 2 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-3. In some embodiments, L¹ is —CH₂NH—[CH₂CH₂O]₂—[CH₂]₂—C≡CH or —CH₂NH—[CH₂CH₂O]₃—[CH₂]₂—C≡CH.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ-arylene-C≡CH. In some embodiments, L¹ is —(C₁₋₃ alkylene)-NH(CH₂CH₂O)ₘ—(CH₂)ₙ-arylene-C≡CH. In some embodiments, the arylene is optionally substituted phenylene. In some embodiments, the alkylene is optionally substituted with 1 or 2 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-3. In some embodiments, L¹ is

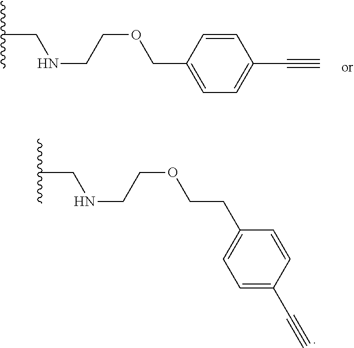 [SMILES: *CNCCOCc1ccc(C#C)cc1 ; *CNCCOCCc1ccc(C#C)cc1]

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—NH—C(O)O—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-12. In some embodiments, n is an integer of 0-8. In some embodiments, L¹ is —CH₂NH—(CH₂CH₂O)—(CH₂)₂—NHC(O)OC(CH₃)₃, —CH₂NH—(CH₂CH₂O)—(CH₂)₃—NHC(O)OC(CH₃)₃, —CH₂NH—(CH₂CH₂O)—(CH₂)₅—NHC(O)OC(CH₃)₃, —CH₂NH—(CH₂CH₂O)—(CH₂)₆—NHC(O)OC(CH₃)₃, —CH₂NH—(CH₂CH₂O)—(CH₂)₈—NHC(O)OC(CH₃)₃, —CH₂NH—(CH₂CH₂O)₂—(CH₂)₂—NHC(O)OC(CH₃)₃, or —CH₂NH—[CH₂CH₂O]₃—[CH₂]₂—NHC(O)OC(CH₃)₃.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—NH—C(O)O—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —CH₂NH—[CH₂CH₂O]₃—[CH₂]₂—NHC(O)OC(CH₃)₃.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—(C₃₋₈ cycloalkylene)-NH—C(O)O—(C₁₋₆ alkyl) or —(C₁₋₆ alkylene)-NH(CH₂CH₂O)ₘ—(CH₂)ₙ—(C₃₋₈ cycloalkylene)-(C₁₋₆ alkylene)-NH—C(O)O—(C₁₋₆ alkyl). In some embodiments, the alkylene, cycloalkylene, or alkyl is optionally and independently substituted with 1, 2, or 3 R^{Z}, wherein each R^{Z} is independently —OH, C₁₋₆ alkyl, or halogen. In some embodiments, the C₃₋₈ cycloalkylene is

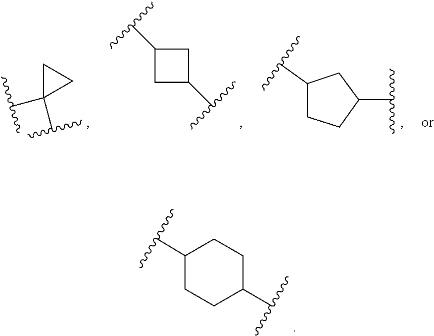 [SMILES: *C1(*)CC1 ; *C1CC(*)C1 ; *C1CCC(*)C1 ; *C1CCC(*)CC1]

In some embodiments, m is an integer of 1-6. In some embodiments, m is an integer of 1-4. In some embodiments, n is an integer of 0-12. In some embodiments, n is an integer of 0-8. In some embodiments, L¹ is

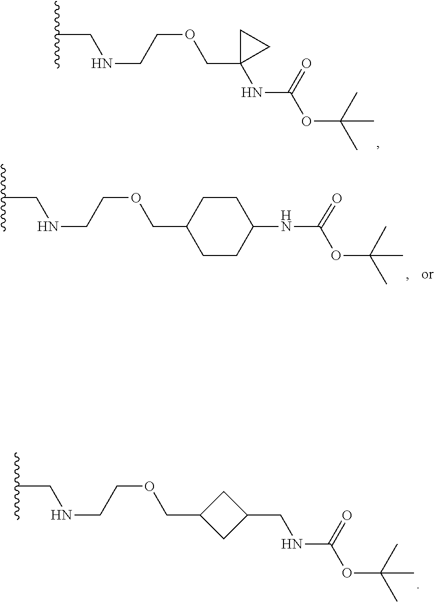 [SMILES: *CNCCOCC1(NC(=O)OC(C)(C)C)CC1 ; *CNCCOCC1CCC(NC(=O)OC(C)(C)C)CC1 ; *CNCCOCC1CC(CNC(=O)OC(C)(C)C)C1]

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ-arylene-NH—C(O)O—(C₁₋₆ alkyl). In some embodiments, the alkylene, arylene, or alkyl is optionally and independently substituted with 1, 2, or 3 R^{Z}, wherein each R^{Z} is independently —OH, C₁₋₆ alkyl, or halogen. In some embodiments, the arylene is phenylene. In embodiments, the phenylene is p-phenylene, m-phenylene o-phenylene. In some embodiments, m is an integer of 1-6. In some embodiments, m is an integer of 1-4. In some embodiments, n is an integer of 0-12. In some embodiments, n is an integer of 0-6. In some embodiments, L¹ is

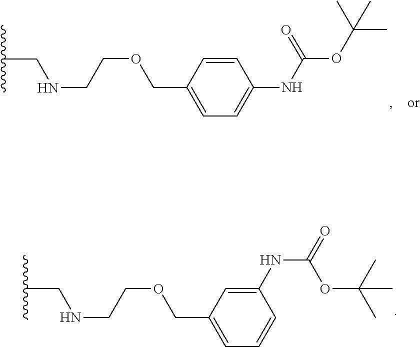 [SMILES: *CNCCOCc1ccc(NC(=O)OC(C)(C)C)cc1 ; *CNCCOCc1cccc(NC(=O)OC(C)(C)C)c1]

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—N(C₁₋₆ alkyl)-C(O)O—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —CH₂NH—[CH₂CH₂O]₃—[CH₂]₃—NCH₃C(O)OC(CH₃)₃, or —CH₂NH—[CH₂CH₂O]₄—[CH₂]₃—NCH₃C(O)OC(CH₃)₃.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ-HET^{A}, or —(C₁₋₆ alkylene)-NH(CH₂CH₂O)ₘ—(CH₂)ₙ—C(O)-HET^{A} wherein HET^{A} is a 4-10 membered heterocycle containing 1-3 heteroatoms selected from N or O, and is optionally substituted with —C(O)—(C₁₋₆ alkyl), —C(O)O—(C₁₋₆ alkyl), —(C₁₋₆ alkyl)-O—C₆₋₁₀ aryl, C₁₋₆ alkyl, —(C₁₋₆ alkylene)-C≡CH, —NH—C(O)O—(C₁₋₆ alkyl), —(C₁₋₆ alkylene)-NH—C(O)O—(C₁₋₆ alkyl), —C₁₋₆ alkoxy, —(C₁₋₆ alkylene)-C₁₋₆ alkoxy, or -arylene-C(O)O—(C₁₋₆ alkyl). In some embodiments, the alkylene is optionally substituted with 1, 2, or 3 R^{Z}, wherein each R^{Z} is independently —OH, C₁₋₆ alkyl, or halogen. In some embodiments, m is an integer of 1-6. In some embodiments, m is an integer of 1-4. In some embodiments, n is an integer of 0-12. In some embodiments, n is an integer of 0-6. In some embodiments, HET^{A} is azetidinyl, pyrrolidinyl, piperidinyl, piperazinyl, or oxetanyl optionally substituted with —C(O)—(C₁₋₆ alkyl), —C(O)O—(C₁₋₆ alkyl), —(C₁₋₆ alkyl)-O—C₆₋₁₀ aryl, —(C₁₋₆ alkylene)-C≡CH, —NH—C(O)O—(C₁₋₆ alkyl), —(C₁₋₆ alkylene)-NH—C(O)O—(C₁₋₆ alkyl), —O—C₁₋₆ alkyl, or —(C₁₋₆ alkylene)-O—(C₁₋₆ alkyl). In some embodiments, HET^{A} is azetidinyl, pyrrolidinyl, piperidinyl, piperazinyl, or oxetanyl optionally substituted with —C(O)—(C₁₋₆ alkyl), —C(O)O—(C₁₋₆ alkyl), or —(C₁₋₆ alkyl)-O—C₆₋₁₀ aryl. In some embodiments, HET^{A} is

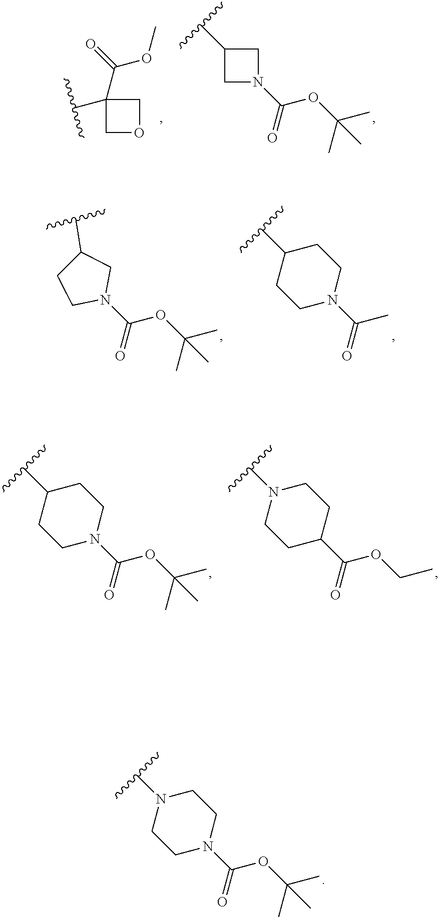

In some embodiments, L¹ is

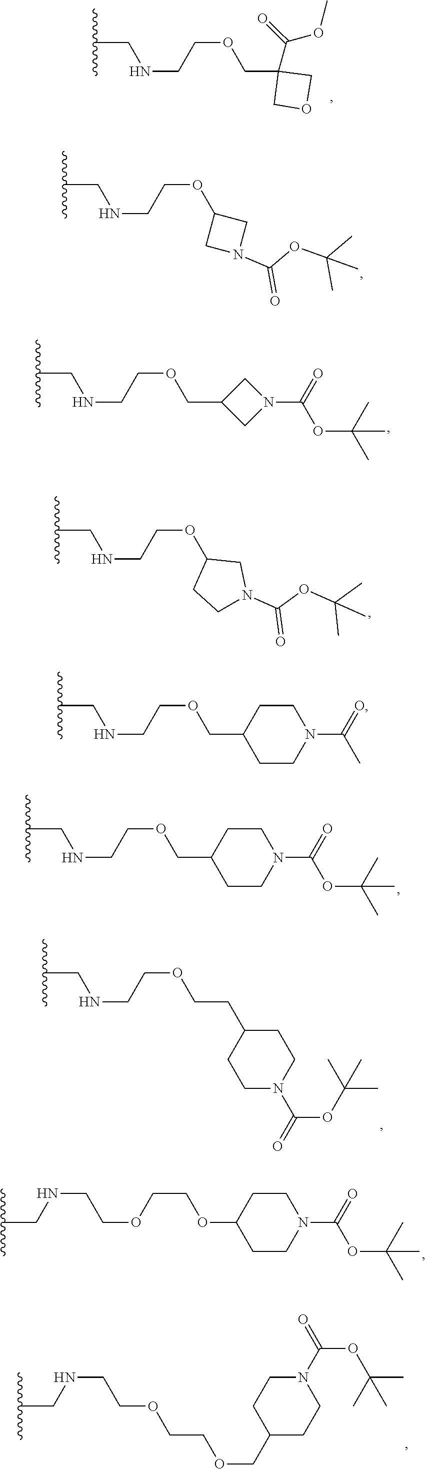

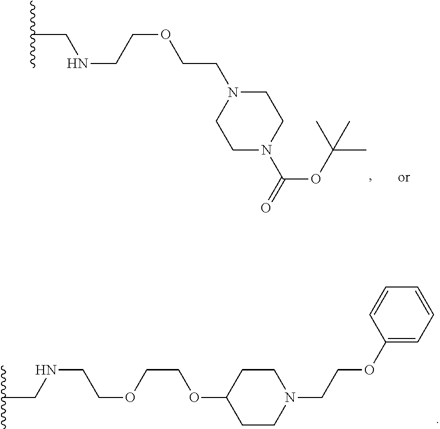 [SMILES: *CNCCOCCN1CCN(C(=O)OC(C)(C)C)CC1 ; *CNCCOCCOC1CCN(CCOc2ccccc2)CC1]

In some embodiments, L¹ is

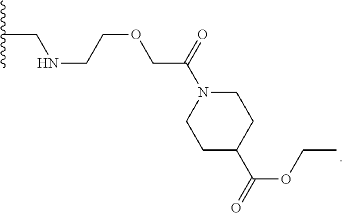 [SMILES: *CNCCOCC(=O)N1CCC(C(=O)OCC)CC1]

In some embodiments, HET^{A} is azetidinyl, pyrrolidinyl, piperidinyl, piperazinyl, or oxetanyl optionally substituted with —O—C₁₋₆ alkyl or —(C₁₋₆ alkylene)-O—(C₁₋₆ alkyl). In some embodiments, HET^{A} is

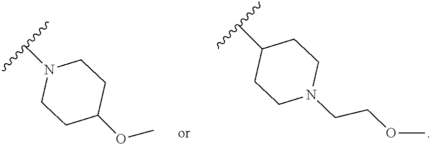

In some embodiments, L¹ is

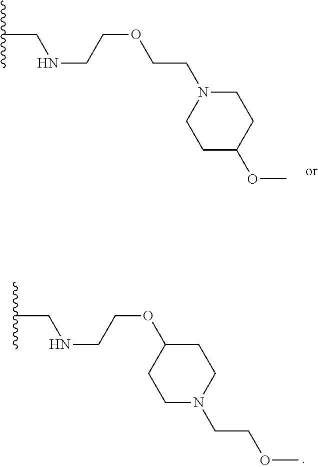 [SMILES: *CNCCOCCN1CCC(OC)CC1 ; *CNCCOC1CCN(CCOC)CC1]

In some embodiments, HET^{A} is azetidinyl, pyrrolidinyl, piperidinyl, piperazinyl, or oxetanyl optionally substituted with —(C₁₋₆ alkylene)-C≡CH. In some embodiments, HET^{A} is

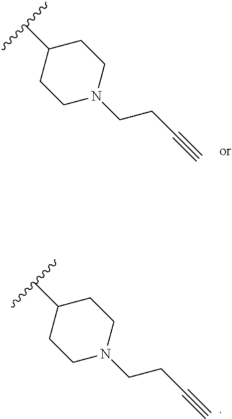 [SMILES: *C1CCN(CCC#C)CC1 ; *C1CCN(CCC#C)CC1]

In some embodiments, L¹ is

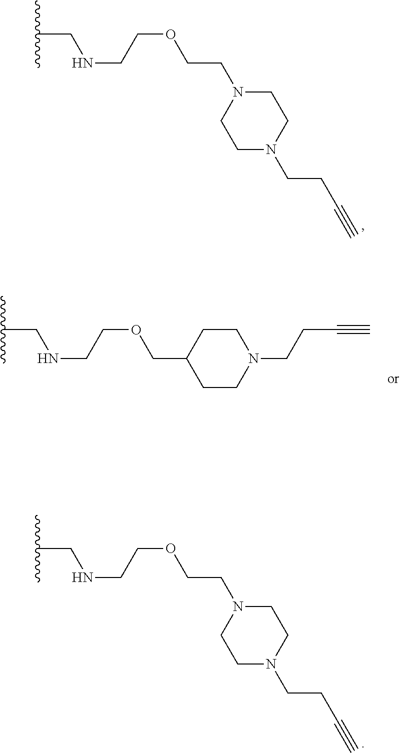 [SMILES: *CNCCOCCN1CCN(CCC#C)CC1 ; *CNCCOCC1CCN(CCC#C)CC1 ; *CNCCOCCN1CCN(CCC#C)CC1]

In some embodiments, HET^{A} is azetidinyl, pyrrolidinyl, piperidinyl, piperazinyl, or oxetanyl optionally substituted with —NH—C(O)O—(C₁₋₆ alkyl) or —(C₁₋₆ alkylene)-NH—C(O)O—(C₁₋₆ alkyl). In some embodiments, HET^{A} is

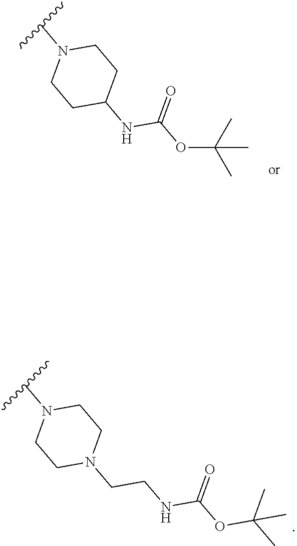 [SMILES: *N1CCC(NC(=O)OC(C)(C)C)CC1 ; *N1CCN(CCNC(=O)OC(C)(C)C)CC1]

In some embodiments, L¹ is

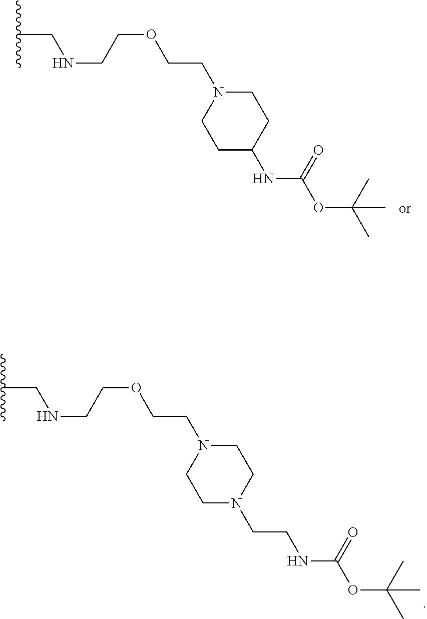 [SMILES: *CNCCOCCN1CCC(NC(=O)OC(C)(C)C)CC1 ; *CNCCOCCN1CCN(CCNC(=O)OC(C)(C)C)CC1]

In some embodiments, HET^{A} is azetidinyl, pyrrolidinyl, piperidinyl, piperazinyl, or oxetanyl optionally substituted with -arylene-C(O)O—(C₁₋₆ alkyl). In some embodiments, the arylene is optionally substituted phenylene. In some embodiments, HET^{A} is

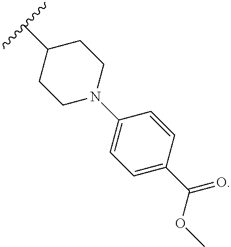 [SMILES: *C1CCN(c2ccc(C(=O)OC)cc2)CC1]

In some embodiments, L¹ is

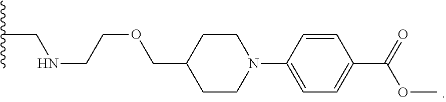 [SMILES: *CNCCOCC1CCN(c2ccc(C(=O)OC)cc2)CC1]

In some embodiments, HET^{A} is piperidinyl optionally substituted with —C(O)—(C₁₋₆ alkyl), —C(O)O—(C₁₋₆ alkyl), or —(C₁₋₆ alkyl)-O—C₆₋₁₀ aryl. In some embodiments, HET^{A} is

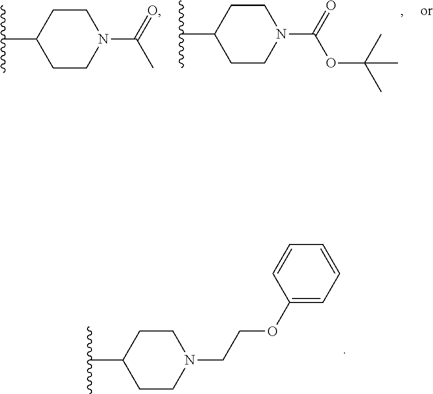

In some embodiments, L¹ is

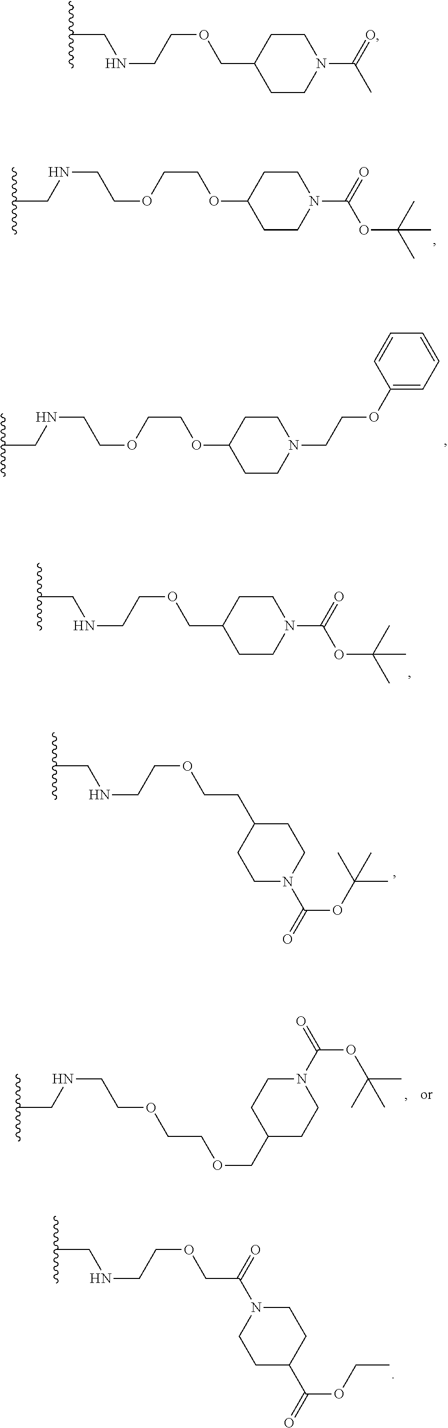

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ-HET^{A}, wherein HET^{A} is a 4-10 membered heterocycle containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl. In some embodiments, the alkylene is optionally substituted with 1, 2, or 3 R^{Z}, wherein each R^{Z} is independently —OH, C₁₋₆ alkyl, or halogen. In some embodiments, HET^{A} is piperidinyl optionally substituted with —C(O)—(C₁₋₆ alkyl), —C(O)O—(C₁₋₆ alkyl), or —(C₁₋₆ alkyl)-O—C₆₋₁₀ aryl. In some embodiments, HET^{A} is

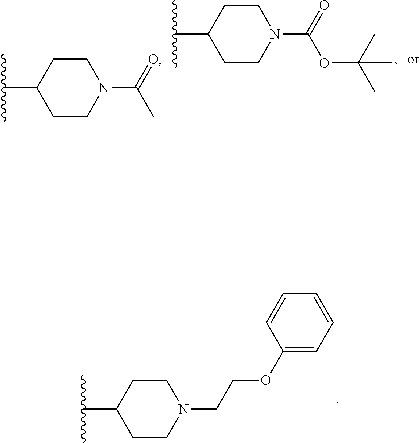

In some embodiments, L¹ is

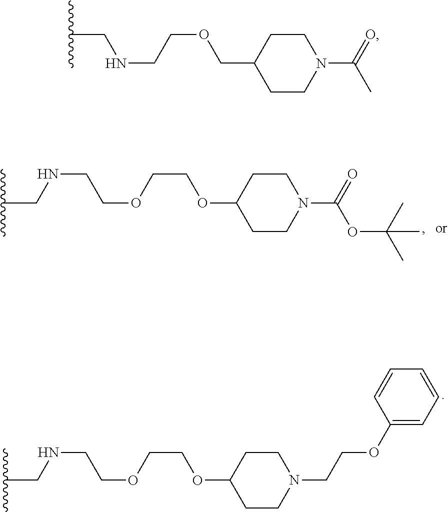 [SMILES: *CNCCOCC1CCN(C(C)=O)CC1 ; *CNCCOCCOC1CCN(C(=O)OC(C)(C)C)CC1 ; *CNCCOCCOC1CCN(CCOc2ccccc2)CC1]

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—NH—(C₁₋₆ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —(C₁₋₆ alkylene)-NH(CH₂CH₂O)ₘ—(CH₂)ₙ—NHCH₃. In some embodiments, m is an integer of 1-6. In some embodiments, m is an integer of 1-4. In some embodiments, n is an integer of 0-12. In some embodiments, n is an integer of 0-6. In some embodiments, L¹ is —CH₂NH—[CH₂CH₂O]₂—[CH₂]₂—NHCH₃, or —CH₂NH—[CH₂CH₂O]₃—[CH₂]₂—NHCH₃.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—C(O)O—(C₁₋₆ alkyl). In some embodiments, L¹ is —(C₁₋₆ alkylene)-NH(CH₂CH₂O)ₘ—(CH₂)ₙ—C(O)O—(C₁₋₄ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —(C₁₋₆ alkylene)-NH(CH₂CH₂O)ₘ—(CH₂)ₙ—C(O)OCH₃ or —(C₁₋₆ alkylene)-NH(CH₂CH₂O)ₘ—(CH₂)ₙ—C(O)OC(CH₃)₃. In some embodiments, m is an integer of 1-6. In some embodiments, m is an integer of 1-4. In some embodiments, n is an integer of 0-12. In some embodiments, n is an integer of 0-8. In some embodiments, L¹ is —CH₂NH—[CH₂CH₂O]₂—[CH₂]₂—C(O)OCH₃, —CH₂NH—(CH₂CH₂O)₃—(CH₂)₂—C(O)OCH₃, —CH₂NH(CH₂CH₂O)—(CH₂)—C(O)OC(CH₃)₃, —CH₂NH(CH₂CH₂O)—(CH₂)₂—C(O)OC(CH₃)₃, —CH₂NH(CH₂CH₂O)—(CH₂)₃—C(O)OC(CH₃)₃, —CH₂NH(CH₂CH₂O)—(CH₂)₄—C(O)OC(CH₃)₃, —CH₂NH(CH₂CH₂O)—(CH₂)₅—C(O)OC(CH₃)₃, —CH₂NH(CH₂CH₂O)—(CH₂)₈—C(O)OC(CH₃)₃, or —CH₂NH(CH₂CH₂O)₂—(CH₂)₂—C(O)OC(CH₃)₃.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—C(O)O—(C₁₋₆ alkyl). In some embodiments, L¹ is —(C₁₋₆ alkylene)-NH(CH₂CH₂O)ₘ—(CH₂)ₙ—C(O)O—(C₁₋₃ alkyl). In some embodiments, the alkylene or alkyl is optionally and independently substituted with 1, 2, or 3 R^{Z}, wherein each R^{Z} is independently —OH, C₁₋₆ alkyl, or halogen. In some embodiments, L¹ is —(C₁₋₆ alkylene)-NH(CH₂CH₂O)ₘ—(CH₂)ₙ—C(O)OCH₃. In some embodiments, L¹ is —CH₂NH—[CH₂CH₂O]₂—[CH₂]₂—C(O)OCH₃.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)₁—O—C₆-10 aryl. In some embodiments, m is an integer of 1-6. In some embodiments, m is an integer of 1-4. In some embodiments, n is an integer of 0-12. In some embodiments, n is an integer of 0-6. In some embodiments, the alkylene or aryl is optionally and independently substituted with 1, 2, or 3 R^{Z}, wherein each R^{Z} is independently —OH, C₁₋₆ alkyl, or halogen. In some embodiments, L¹ is

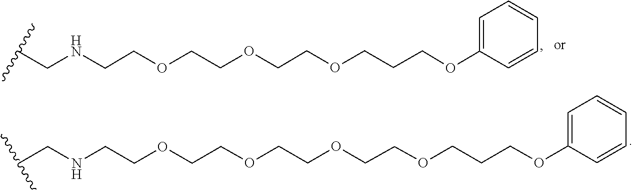 [SMILES: *CNCCOCCOCCOCCCOc1ccccc1 ; *CNCCOCCOCCOCCOCCCOc1ccccc1]

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ-arylene-C(O)O—(C₁₋₆ alkyl). In some embodiments, the alkylene, arylene or alkyl is optionally and independently substituted with 1, 2, or 3 R^{Z}, wherein each R^{Z} is independently —OH, C₁₋₆ alkyl, or halogen. In some embodiments, the arylene is phenylene. In embodiments, the phenylene is p-phenylene, m-phenylene o-phenylene. In some embodiments, m is an integer of 1-6. In some embodiments, m is an integer of 1-4. In some embodiments, n is an integer of 0-12. In some embodiments, n is an integer of 0-6. In some embodiments, L¹ is

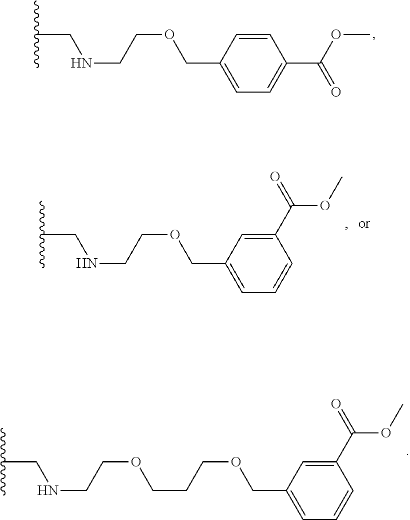 [SMILES: *CNCCOCc1ccc(C(=O)OC)cc1 ; *CNCCOCc1cccc(C(=O)OC)c1 ; *CNCCOCCCOCc1cccc(C(=O)OC)c1]

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ-heteroarylene-C(O)O—(C₁₋₆ alkyl). In some embodiments, the alkylene, heteroarylene or alkyl is optionally and independently substituted with 1, 2, or 3 R^{Z}, wherein each R^{Z} is independently —OH, C₁₋₆ alkyl, or halogen. In some embodiments, the heteroarylene is pyridinylene. In some embodiments, m is an integer of 1-6. In some embodiments, m is an integer of 1-4. In some embodiments, n is an integer of 0-12. In some embodiments, n is an integer of 0-6. In some embodiments, L¹ is

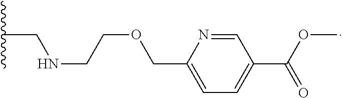 [SMILES: *CNCCOCc1ccc(C(=O)OC)cn1]

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—X^{H}, wherein X^{H} is halogen. In some embodiments, X^{H} is chloro or bromo. In some embodiments, m is an integer of 1-6. In some embodiments, m is an integer of 1-4. In some embodiments, n is an integer of 0-12. In some embodiments, n is an integer of 0-6. In some embodiments, L¹ is —CH₂NH—(CH₂CH₂O)₂—(CH₂)₃—C₁.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —O—CH₂CH(OH)—(C₁₋₆ alkylene)-NH(CH₂CH₂O)ₘ—(CH₂)ₙ—C≡CH. In some embodiments, m is an integer of 1-6. In some embodiments, m is an integer of 1-4. In some embodiments, n is an integer of 0-12. In some embodiments, n is an integer of 0-6. In some embodiments, L¹ is —O—CH₂CH(OH)—CH₂—NH(CH₂CH₂O)₂—(CH₂)₂—C≡CH.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —O—CH₂CH(OH)—(C₁₋₆ alkylene)-NH(CH₂CH₂O)ₘ—(CH₂)ₙ—C(O)O—(C₁₋₆ alkyl). In some embodiments, m is an integer of 1-6. In some embodiments, m is an integer of 1-4. In some embodiments, n is an integer of 0-12. In some embodiments, n is an integer of 0-6. In some embodiments, L¹ is —O—CH₂CH(OH)—CH₂—NH(CH₂CH₂O)—(CH₂)₂—C(O)OCH₃.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, L¹ is —(C₁₋₆ alkylene)-NH(CH₂CH₂O)ₘ—(CH₂)ₙ—(CH₂CH₂O)ₘ—(CH₂)ₙ—C(O)O—(C₁₋₆ alkyl). In some embodiments, each m is independently an integer of 1-12; and each n is independently an integer of 0-12. In some embodiments, each m is an integer of 1-6, 1-4, or 2-3. In some embodiments, each n is an integer of 0-6, 1-5, or 2-3. In some embodiments, L¹ is —CH₂NH(CH₂CH₂O)—(CH₂)—(CH₂CH₂O)—(CH₂)—C(O)OC(CH₃)₃.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, wherein L¹ is:
—(C₁₋₆ alkylene)-NH(CH₂CH₂O)ₘ—(CH₂)ₙ—C≡CH,
—(C₁₋₆ alkylene)-NH(CH₂CH₂O)ₘ—(CH₂)ₙ-arylene-C≡CH,
—(C₁₋₆ alkylene)-NH(CH₂CH₂O)ₘ—(CH₂)ₙ—NH—C(O)O—(C₁₋₆ alkyl),
—(C₁₋₆ alkylene)-NH(CH₂CH₂O)ₘ—(CH₂)ₙ—N(C₁₋₆ alkyl)-C(O)O—(C₁₋₆ alkyl),
—(C₁₋₆ alkylene)-NH(CH₂CH₂O)ₘ—(CH₂)ₙ-arylene-C(O)O—(C₁₋₆ alkyl),
—(C₁₋₆ alkylene)-NH(CH₂CH₂O)ₘ—(CH₂)ₙ-arylene-NH—C(O)O—(C₁₋₆ alkyl),
—(C₁₋₆ alkylene)-NH(CH₂CH₂O)ₘ—(CH₂)ₙ—(C₃₋₈ cycloalkylene)-NH—C(O)O—(C₁₋₆ alkyl),
—(C₁₋₆ alkylene)-NH(CH₂CH₂O)ₘ—(CH₂)ₙ—(C₃₋₈ cycloalkylene)-(C₁₋₆ alkylene)-NH—C(O)O—(C₁₋₆ alkyl),
—(C₁₋₆ alkylene)-NH(CH₂CH₂O)ₘ—(CH₂)ₙ-heteroarylene-C(O)O—(C₁₋₆ alkyl),
—(C₁₋₆ alkylene)-NH(CH₂CH₂O)ₘ—(CH₂)ₙ-HET^{A}
—(C₁₋₆ alkylene)-NH(CH₂CH₂O)ₘ—(CH₂)ₙ—C(O)-HET^{A}
—(C₁₋₆ alkylene)-NH(CH₂CH₂O)ₘ—(CH₂)ₙ—NH—(C₁₋₆ alkyl),
—(C₁₋₆ alkylene)-NH(CH₂CH₂O)ₘ—(CH₂)ₙ—C(O)O—(C₁₋₆ alkyl),
—(C₁₋₆ alkylene)-NH(CH₂CH₂O)ₘ—(CH₂)ₙ—O—C₆₋₁₀ aryl,
—(C₁₋₆ alkylene)-NH(CH₂CH₂O)ₘ—(CH₂)ₙ—X^{H},
—O—CH₂CH(OH)—(C₁₋₆ alkylene)-NH(CH₂CH₂O)ₘ—(CH₂)ₙ—C≡CH,
—O—CH₂CH(OH)—(C₁₋₆ alkylene)-NH(CH₂CH₂O)ₘ—(CH₂)ₙ—C(O)O—(C₁₋₆ alkyl), or
—(C₁₋₆ alkylene)-NH(CH₂CH₂O)ₘ—(CH₂)ₙ—(CH₂CH₂O)ₘ—(CH₂)ₙ—C(O)O—(C₁₋₆ alkyl),
wherein HET^{A} is a 4-10 membered heterocycle containing 1-3 heteroatoms selected from N or O, and is optionally substituted with —C(O)—(C₁₋₆ alkyl), —C(O)O—(C₁₋₆ alkyl), —(C₁₋₆ alkyl)-O—C₆₋₁₀ aryl, C₁₋₆ alkyl, —(C₁₋₆ alkylene)-C≡CH, —NH—C(O)O—(C₁₋₆ alkyl), —(C₁₋₆ alkylene)-NH—C(O)O—(C₁₋₆ alkyl), —C₁₋₆ alkoxy, —(C₁₋₆ alkylene)-C₁₋₆ alkoxy, or -arylene-C(O)O—(C₁₋₆ alkyl);
X^{H} is halogen;
each m is independently an integer of 1-12; and
each n is independently an integer of 0-12.

In some embodiments, HET^{A} is azetidinyl, pyrrolidinyl, piperidinyl, piperazinyl, or oxetanyl optionally substituted with —C(O)—(C₁₋₆ alkyl), —C(O)O—(C₁₋₆ alkyl), —(C₁₋₆ alkyl)-O—C₆₋₁₀ aryl, —(C₁₋₆ alkylene)-C≡CH, —NH—C(O)O—(C₁₋₆ alkyl), —(C₁₋₆ alkylene)-NH—C(O)O—(C₁₋₆ alkyl), —O—C₁₋₆ alkyl, or —(C₁₋₆ alkylene)-O—(C₁₋₆ alkyl).

In some embodiments, each m is independently an integer of 1-12, 1-6, or 1-4. In some embodiments, each n is independently an integer of 0-12, 0-8, or 0-3.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, wherein L¹ is:
—(C₁₋₆ alkylene)-NH(CH₂CH₂O)ₘ—(CH₂)ₙ—C≡CH,
—(C₁₋₆ alkylene)-NH(CH₂CH₂O)ₘ—(CH₂)ₙ—NH—C(O)O—(C₁₋₆ alkyl),
—(C₁₋₆ alkylene)-NH(CH₂CH₂O)ₘ—(CH₂)ₙ—N(C₁₋₆ alkyl)-C(O)O—(C₁₋₆ alkyl),
—(C₁₋₆ alkylene)-NH(CH₂CH₂O)ₘ—(CH₂)ₙ-HET^{A}
—(C₁₋₆ alkylene)-NH(CH₂CH₂O)ₘ—(CH₂)ₙ—NH—(C₁₋₆ alkyl),
—(C₁₋₆ alkylene)-NH(CH₂CH₂O)ₘ—(CH₂)ₙ—C(O)O—(C₁₋₆ alkyl), or
—(C₁₋₆ alkylene)-NH(CH₂CH₂O)ₘ—(CH₂)ₙ—O—C₆₋₁₀ aryl;
wherein the alkylene, alkyl, or aryl is optionally and independently substituted with 1, 2, or 3 R^{Z}, wherein each R^{Z} is independently —OH, C₁₋₆ alkyl, or halogen;
HET^{A} is a 4-10 membered heterocycle containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl;
m is an integer of 1-12; and
n is an integer of 0-6.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, wherein L¹ is
—CH₂NH—(CH₂CH₂O)—(CH₂)₂—C≡CH,
—CH₂NH—(CH₂CH₂O)—(CH₂)₅—C≡CH,
—CH₂NH—(CH₂CH₂O)—(CH₂)₆—C≡CH,
—CH₂NH—(CH₂CH₂O)—(CH₂)₈—C≡CH,
—CH₂NH—(CH₂CH₂O)₂—(CH₂)—C≡CH,
—CH₂NH—(CH₂CH₂O)₂—(CH₂)₂—C≡CH,
—CH₂NH—(CH₂CH₂O)₃—(CH₂)—C≡CH,
—CH₂NH—(CH₂CH₂O)₃—(CH₂)₂—C≡CH,

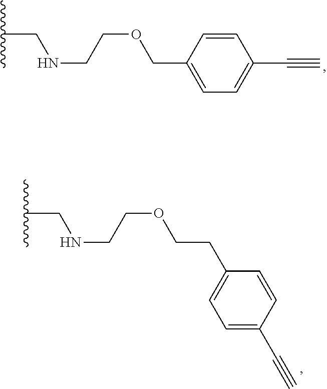 [SMILES: *CNCCOCc1ccc(C#C)cc1 ; *CNCCOCCc1ccc(C#C)cc1]

—CH₂NH—(CH₂CH₂O)—(CH₂)₂—NHC(O)OC(CH₃)₃,
—CH₂NH—(CH₂CH₂O)—(CH₂)₃—NHC(O)OC(CH₃)₃,
—CH₂NH—(CH₂CH₂O)—(CH₂)₅—NHC(O)OC(CH₃)₃,
—CH₂NH—(CH₂CH₂O)—(CH₂)₆—NHC(O)OC(CH₃)₃,
—CH₂NH—(CH₂CH₂O)—(CH₂)₈—NHC(O)OC(CH₃)₃,
—CH₂NH—(CH₂CH₂O)₂—(CH₂)₂—NHC(O)OC(CH₃)₃,
—CH₂NH—(CH₂CH₂O)₃—(CH₂)₂—NHC(O)OC(CH₃)₃,

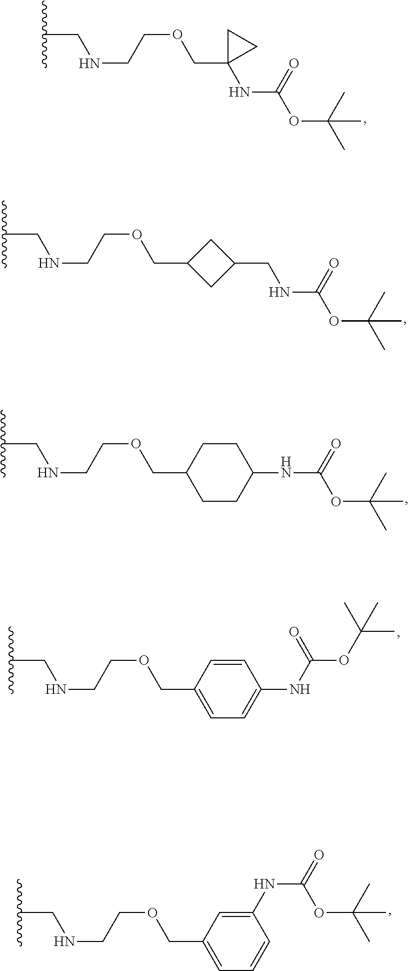 [SMILES: *CNCCOCC1(NC(=O)OC(C)(C)C)CC1 ; *CNCCOCC1CC(CNC(=O)OC(C)(C)C)C1 ; *CNCCOCC1CCC(NC(=O)OC(C)(C)C)CC1 ; *CNCCOCc1ccc(NC(=O)OC(C)(C)C)cc1 ; *CNCCOCc1cccc(NC(=O)OC(C)(C)C)c1]

—CH₂NH—(CH₂CH₂O)₃—(CH₂)₃—NCH₃C(O)OC(CH₃)₃,
—CH₂NH—(CH₂CH₂O)₄—(CH₂)₃—NCH₃C(O)OC(CH₃)₃,

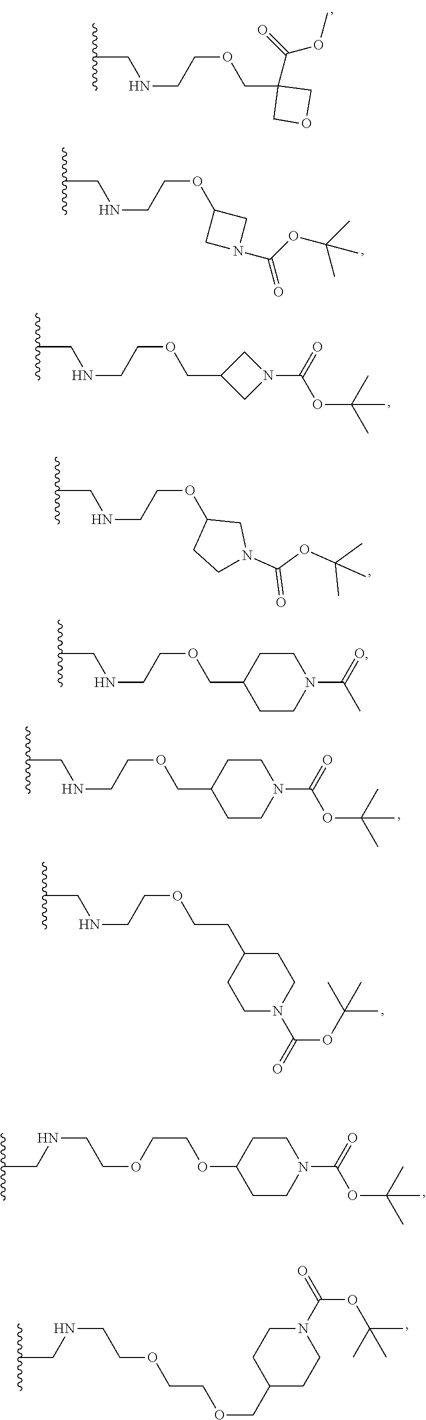

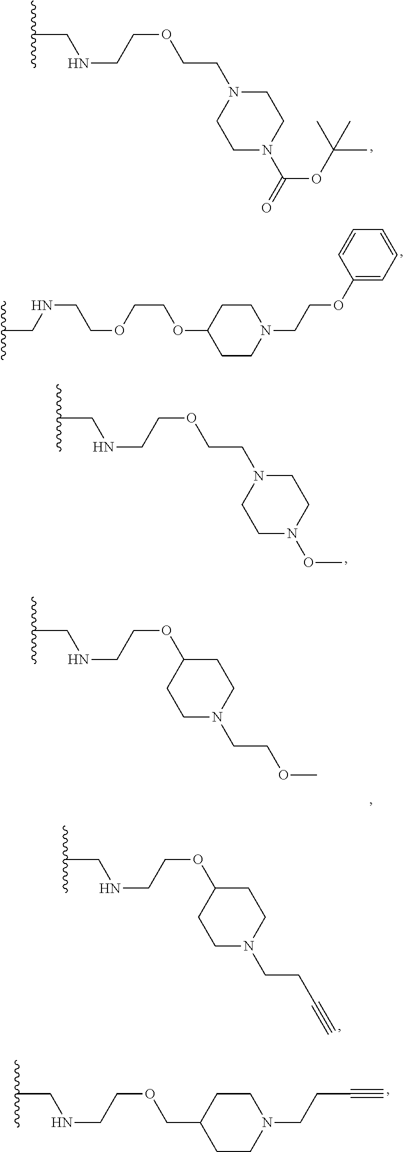 [SMILES: *CNCCOCCN1CCN(C(=O)OC(C)(C)C)CC1 ; *CNCCOCCOC1CCN(CCOc2ccccc2)CC1 ; *CNCCOCCN1CCN(OC)CC1 ; *CNCCOC1CCN(CCOC)CC1 ; *CNCCOC1CCN(CCC#C)CC1 ; *CNCCOCC1CCN(CCC#C)CC1]

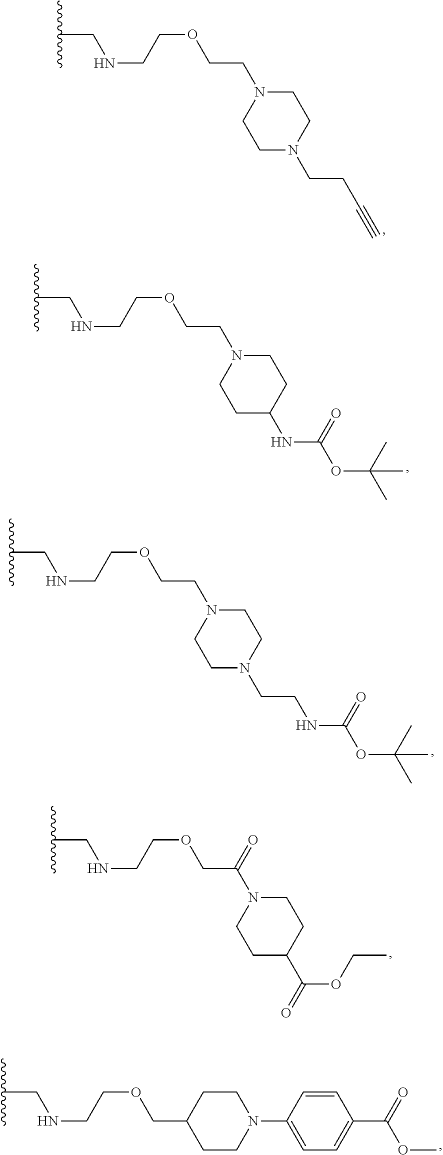 [SMILES: *CNCCOCCN1CCN(CCC#C)CC1 ; *CNCCOCCN1CCC(NC(=O)OC(C)(C)C)CC1 ; *CNCCOCCN1CCN(CCNC(=O)OC(C)(C)C)CC1 ; *CNCCOCC(=O)N1CCC(C(=O)OCC)CC1 ; *CNCCOCC1CCN(c2ccc(C(=O)OC)cc2)CC1]

—CH₂NH—(CH₂CH₂O)₂—(CH₂)₂—NHCH₃,
—CH₂NH—(CH₂CH₂O)₃—(CH₂)₂—NHCH₃,
—CH₂NH—(CH₂CH₂O)₂—(CH₂)₂—C(O)OCH₃,
—CH₂NH—(CH₂CH₂O)₃—(CH₂)₂—C(O)OCH₃,

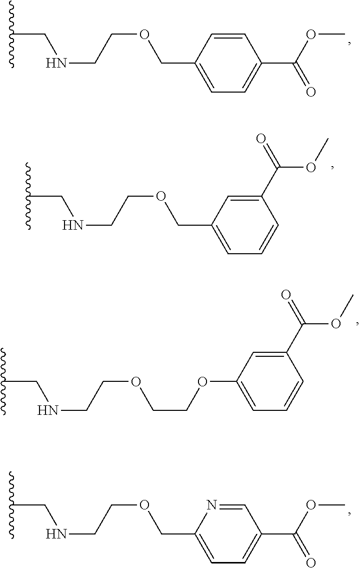 [SMILES: *CNCCOCc1ccc(C(=O)OC)cc1 ; *CNCCOCc1cccc(C(=O)OC)c1 ; *CNCCOCCOc1cccc(C(=O)OC)c1 ; *CNCCOCc1ccc(C(=O)OC)cn1]

—CH₂NH(CH₂CH₂O)—(CH₂)—C(O)OC(CH₃)₃,
—CH₂NH(CH₂CH₂O)—(CH₂)₂—C(O)OC(CH₃)₃,
—CH₂NH(CH₂CH₂O)—(CH₂)₃—C(O)OC(CH₃)₃,
—CH₂NH(CH₂CH₂O)—(CH₂)₄—C(O)OC(CH₃)₃,
—CH₂NH(CH₂CH₂O)—(CH₂)₄—C(O)OC(CH₃)₃,
—CH₂NH(CH₂CH₂O)—(CH₂)₈—C(O)OC(CH₃)₃,
—CH₂NH(CH₂CH₂O)₂—(CH₂)₂—C(O)OC(CH₃)₃,
—CH₂NH(CH₂CH₂O)—(CH₂)—(CH₂CH₂O)—(CH₂)—C(O)OC(CH₃)₃,

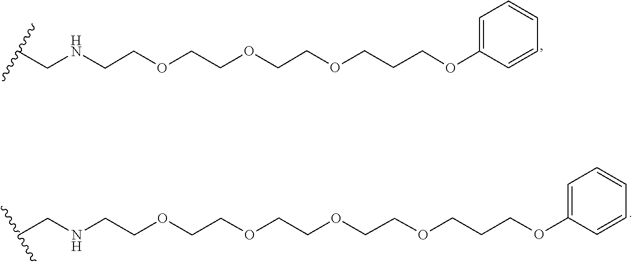 [SMILES: *CNCCOCCOCCOCCCOc1ccccc1 ; *CNCCOCCOCCOCCOCCCOc1ccccc1]

—CH₂NH—(CH₂CH₂O)₂—(CH₂)₃—C₁,
—O—CH₂CH(OH)—CH₂—NH(CH₂CH₂O)₂—(CH₂)₂—C≡CH, or
—O—CH₂CH(OH)—CH₂—NH(CH₂CH₂O)—(CH₂)₂—C(O)OCH₃.

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, wherein L¹ is
—CH₂NH—(CH₂CH₂O)₂—(CH₂)₂—C≡CH,
—CH₂NH—(CH₂CH₂O)₃—(CH₂)₂—C≡CH,
—CH₂NH—(CH₂CH₂O)₃—(CH₂)₂—NHC(O)OC(CH₃)₃,
—CH₂NH—(CH₂CH₂O)₃—(CH₂)₃—NCH₃C(O)OC(CH₃)₃,
—CH₂NH—(CH₂CH₂O)₄—(CH₂)₃—NCH₃C(O)OC(CH₃)₃,

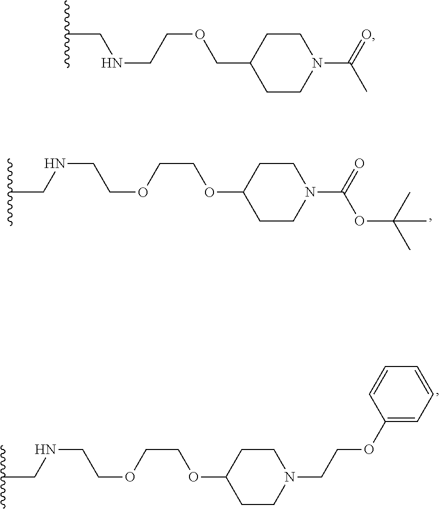 [SMILES: *CNCCOCC1CCN(C(C)=O)CC1 ; *CNCCOCCOC1CCN(C(=O)OC(C)(C)C)CC1 ; *CNCCOCCOC1CCN(CCOc2ccccc2)CC1]

—CH₂NH—(CH₂CH₂O)₂—(CH₂)₂—NHCH₃,
—CH₂NH—(CH₂CH₂O)₃—(CH₂)₂—NHCH₃,
—CH₂NH—(CH₂CH₂O)₂—(CH₂)₂—C(O)OCH₃,

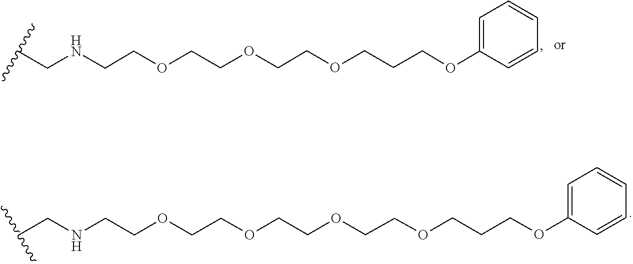 [SMILES: *CNCCOCCOCCOCCCOc1ccccc1 ; *CNCCOCCOCCOCCOCCCOc1ccccc1]

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, wherein L¹ comprises a bivalent moiety selected from:

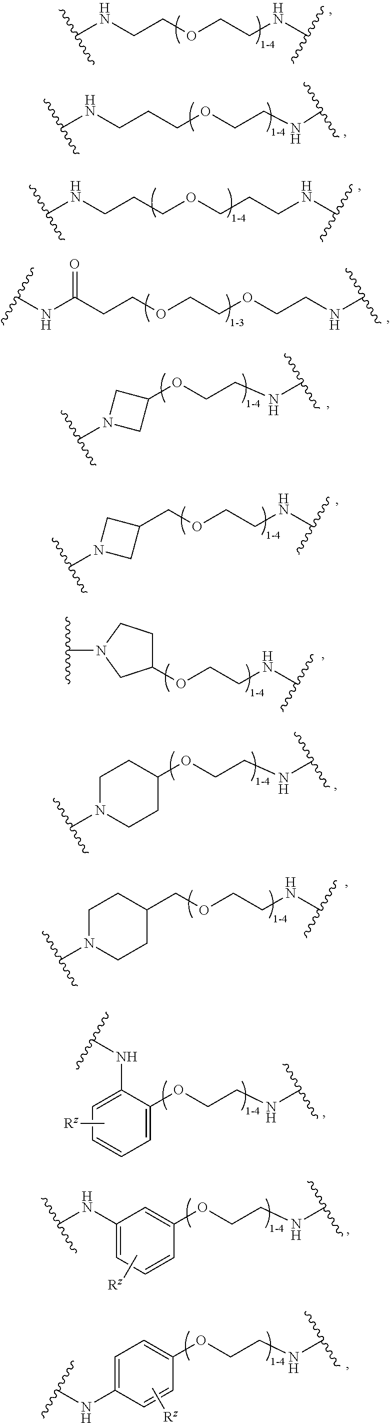

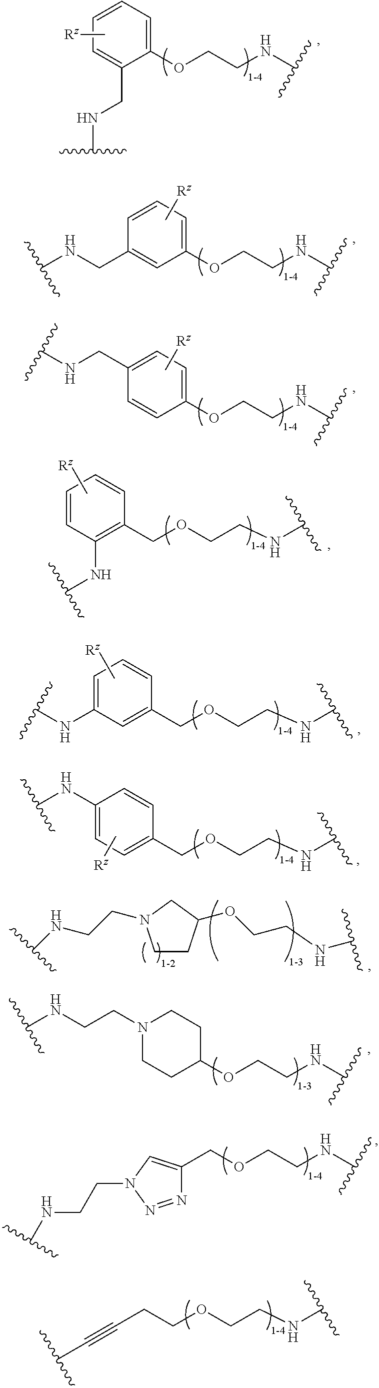

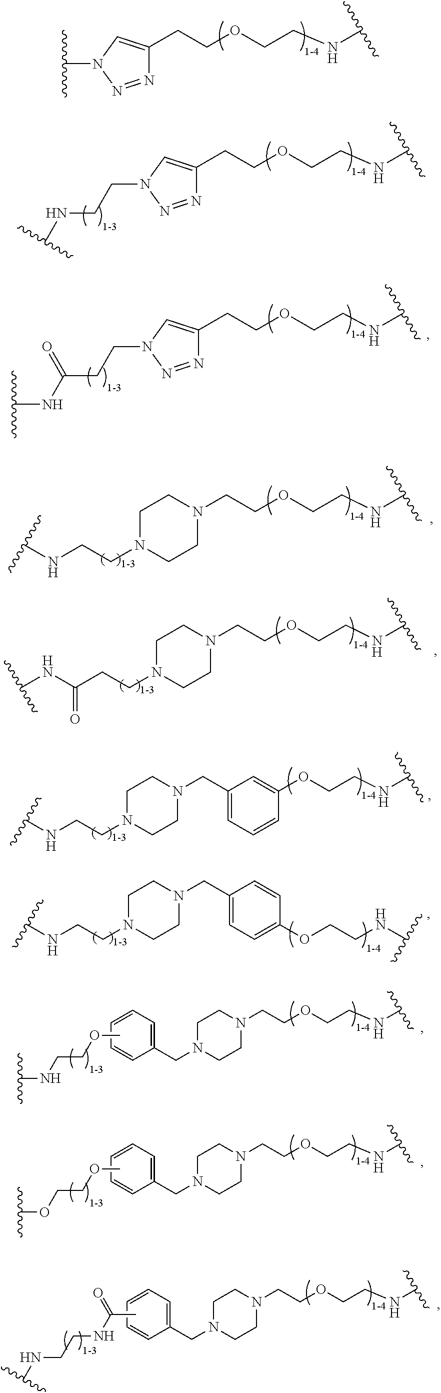

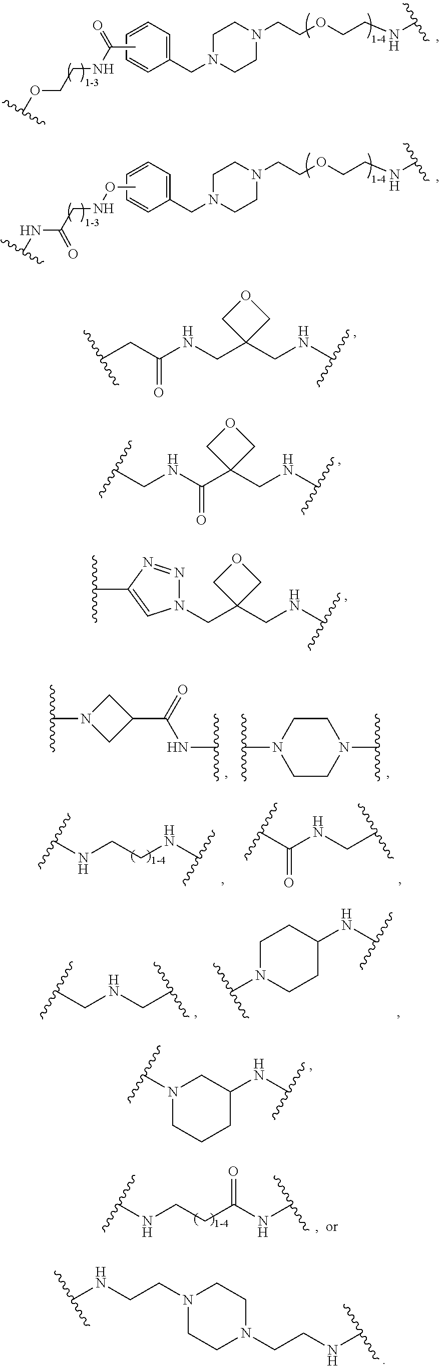

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, wherein L¹ comprises a bivalent moiety selected from:

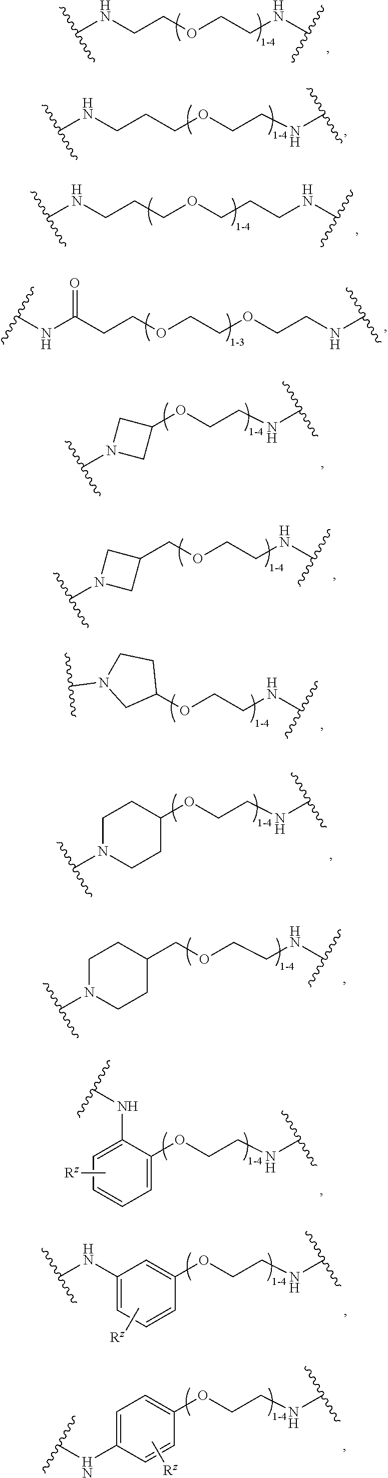

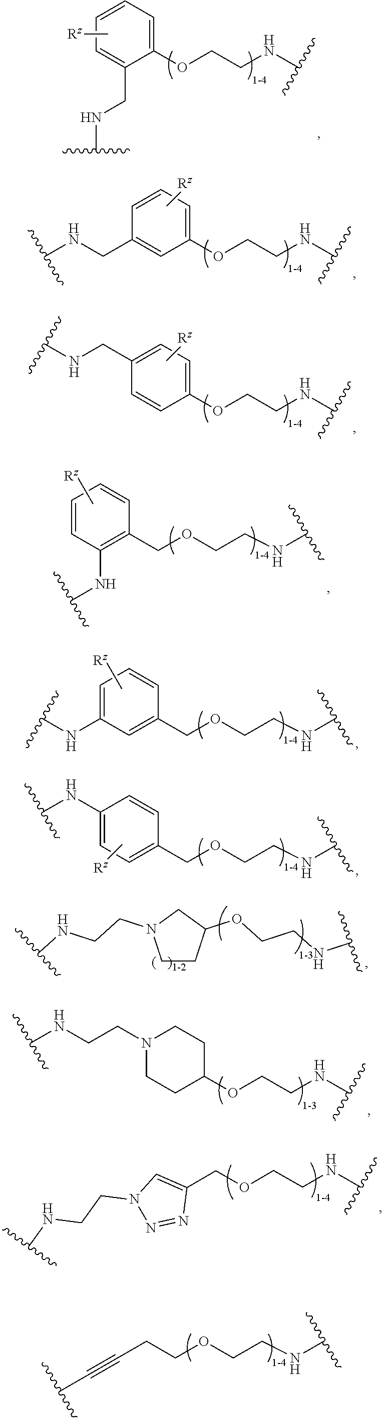

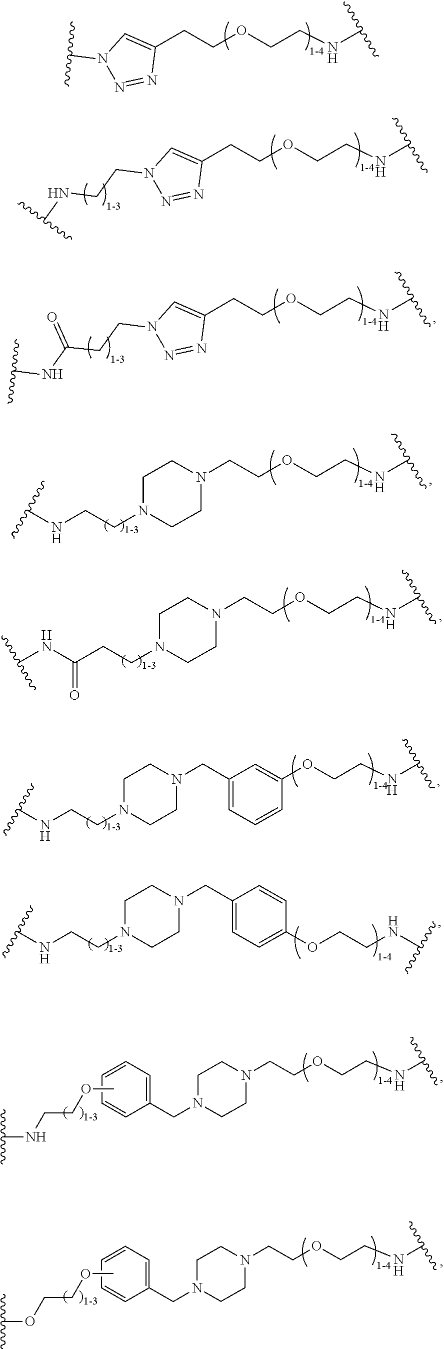

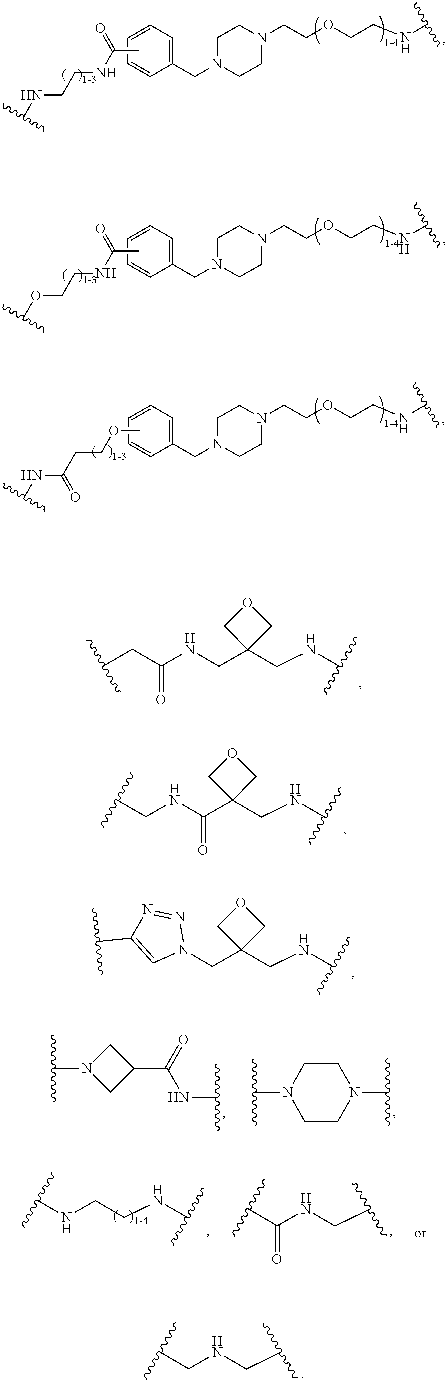

In embodiments, a ligand (“A”) that binds to a protein, a protein aggregate, a protein complex, or a lipid that is targeted for degradation is covalently attached to the compounds of formula (X-II), (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1). In embodiments, the present disclosure provides a compound of formula (X-III):

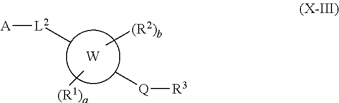

or a pharmaceutically acceptable salt, stereoisomer, or deuterated form thereof,
wherein:
   A is a ligand (e.g., a PBC) that binds to a protein, a protein aggregate, a protein complex, or a lipid;
   ring W is 6,5-fused heteroaryl or 6,5-fused heterocycle ring, wherein each ring W contains 1, 2, or 3 heteroatoms selected from N, O, or S, and at least 1 of the heteroatoms is N or O;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3;
   b is an integer of 0-2; and
   L² is a linker moiety that covalently binds ligand A to ring W.

In some embodiments, L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen.

In embodiments, the present disclosure provides a compound of formula (III)

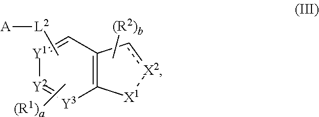

or a pharmaceutically acceptable salt, stereoisomer, or deuterated form thereof,
wherein:
   A is a ligand that binds to a protein, a protein aggregate, a protein complex, or a lipid;
   L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (III-A):

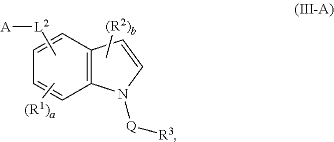

or a pharmaceutically acceptable salt, a stereoisomer, or a deuterated form thereof,
wherein:
   A is a ligand that binds to a protein, a protein aggregate, a protein complex, or a lipid;
   L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (III-A-1), (III-A-2), or (III-A-3):

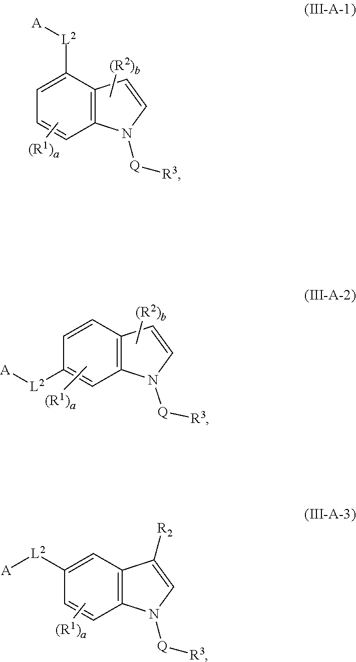

or a pharmaceutically acceptable salt, a stereoisomer, or a deuterated form thereof,
wherein:
   A is a ligand that binds to a protein, a protein aggregate, a protein complex, or a lipid;
   L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (III-B), (III-C), or (III-D):

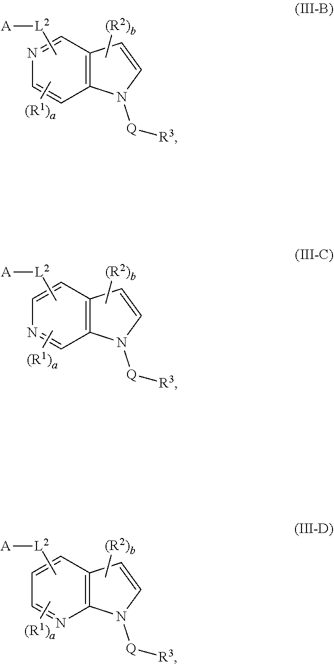

or a pharmaceutically acceptable salt, a stereoisomer, or a deuterated form thereof,
wherein:
   A is a ligand that binds to a protein, a protein aggregate, a protein complex, or a lipid;
   L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (III-B-1), (III-C-1), or (III-D-1):

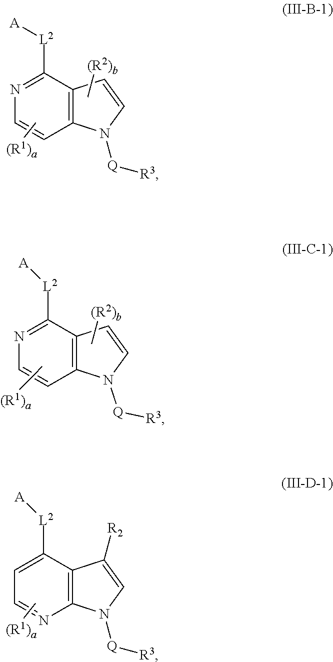

or a pharmaceutically acceptable salt, a stereoisomer, or a deuterated form thereof,
wherein:
   A is a ligand that binds to a protein, a protein aggregate, a protein complex, or a lipid;
   L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (III-E), (III-F), or (III-G):

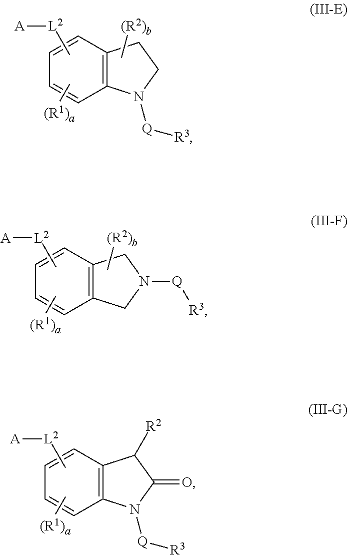

or a pharmaceutically acceptable salt, a stereoisomer, or a deuterated form thereof,
wherein:
   A is a ligand that binds to a protein, a protein aggregate, a protein complex, or a lipid;
   L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

In embodiments, the present disclosure provides a compound of formula (III-E-1), (III-F-1), or (III-G-1):

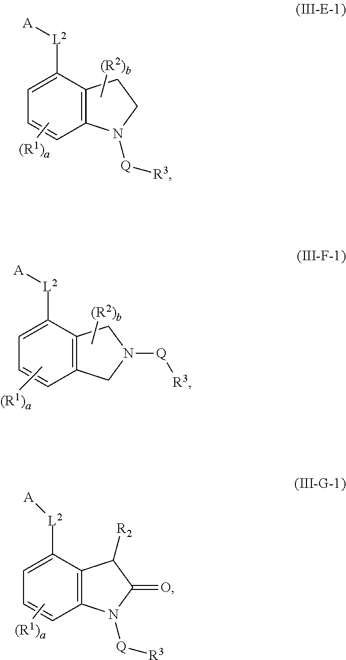

or a pharmaceutically acceptable salt, a stereoisomer, or a deuterated form thereof,
wherein:
   A is a ligand that binds to a protein, a protein aggregate, a protein complex, or a lipid;
   L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

### L²

In embodiments of the compound of formula (X-III), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted. In embodiments, the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen.

In embodiments of the compound of formula (X-III), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —N(H)C(O)—, —N(C₁-C₆ alkyl)C(O)—, —N(C₃—C₈ cycloalkyl)C(O)—, —C(O)N(H)—, —C(O)N(C₁-C₆ alkyl)-, —C(O)N(C₃-C₈ cycloalkyl)-, arylene, heteroarylene, heterocyclylene, or C₃-C₈ cycloalkylene, wherein each arylene or heteroarylene is optionally and independently substituted. In some embodiments, each arylene or heteroarylene is optionally and independently substituted with 1 or 2 R^{Z}, wherein each R^{Z} is independently OH, C₁₋₆ alkyl, or halogen.

In embodiments of the compound of formula (X-III), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, wherein L² is:
—(C₁₋₆ alkylene)-N(H)(CH₂CH₂O)ₘ—(CH₂)ₙ—N(H)—,
—(C₁₋₆ alkylene)-N(H)(CH₂CH₂O)ₘ—(CH₂)ₙ—N(C₁₋₆ alkyl)-,
—(C₁₋₆ alkylene)-N(H)(CH₂CH₂O)ₘ—(CH₂)ₙ—,
—(C₁₋₆ alkylene)-N(H)(CH₂CH₂O)ₘ—(CH₂)ₙ—N(H)—C(O)—(C₁₋₆ alkylene)-O—,
—(C₁₋₆ alkylene)-N(H)(CH₂CH₂O)ₘ—(CH₂)—HET^{B}-, or
—(C₁₋₆ alkylene)-N(H)(CH₂CH₂O)ₘ—(CH₂)ₙ-HET^{B}-(C₁₋₆ alkylene)-,
wherein each alkylene or alkyl is optionally and independently substituted with 1, 2, or 3 R^{Z}, wherein each R^{Z} is independently OH, C₁₋₆ alkyl, or halogen;
HET^{B} is a 4-10 membered heterocyclylene containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl;
m is an integer of 1-12; and
n is an integer of 0-6.

In embodiments of the compound of formula (X-III), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, wherein L² is:
—(C₁₋₆ alkylene)-NH(CH₂CH₂O)ₘ—(CH₂)ₙ—NH—,
—(C₁₋₆ alkylene)-NH(CH₂CH₂O)ₘ—(CH₂)ₙ—N(C₁₋₆ alkyl)-,
—(C₁₋₆ alkylene)-NH(CH₂CH₂O)ₘ—(CH₂)ₙ—,
—(C₁₋₆ alkylene)-NH(CH₂CH₂O)ₘ—(CH₂)ₙ—NH—C(O)—(C₁₋₆ alkylene)-O—, or
—(C₁₋₆ alkylene)-NH(CH₂CH₂O)ₘ—(CH₂)—HET^{B}-,
wherein each alkylene or alkyl is optionally and independently substituted with 1, 2, or 3 R^{Z}, wherein each R^{Z} is independently OH, C₁₋₆ alkyl, or halogen;
HET^{B} is a 4-10 membered heterocyclylene containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl;
m is an integer of 1-12; and
n is an integer of 0-6.

In some embodiments of the compound of formula (X-III), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, L² is:
—(CH₂)—N(H)(CH₂CH₂O)—(CH₂)₂—N(H)—,
—(CH₂)—N(H)(CH₂CH₂O)₂—(CH₂)₂—N(H)—,
—(CH₂)—N(H)(CH₂CH₂O)₃—(CH₂)₂—N(H)—,
—(CH₂)—N(H)(CH₂CH₂O)₃—(CH₂)₃—N(CH₃)—,
—(CH₂)—N(H)(CH₂CH₂O)₄—(CH₂)₃—N(CH₃)—,
—(CH₂)—N(H)(CH₂CH₂O)₃—,
—(CH₂)—N(H)(CH₂CH₂O)₄—,
—(CH₂)—N(H)(CH₂CH₂O)₃—(CH₂)₂—N(H)—C(O)CH₂—O—,
—(CH₂)—N(H)(CH₂CH₂O)₃—(CH₂)₂—,
—(CH₂)—N(H)(CH₂CH₂O)₃—(CH₂)₃—,

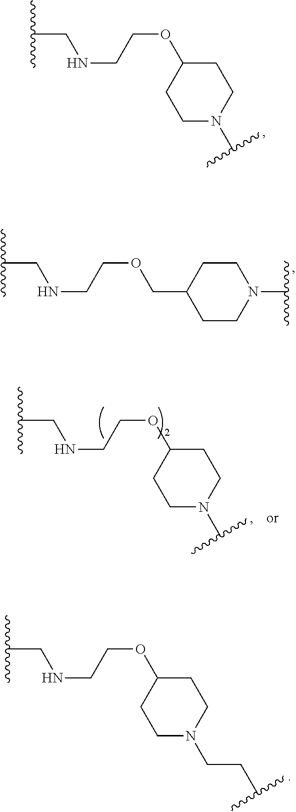

In some embodiments of the compound of formula (X-III), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, L² is:
—(CH₂)—NH(CH₂CH₂O)—(CH₂)₂—NH—,
—(CH₂)—NH(CH₂CH₂O)₂—(CH₂)₂—NH—,
—(CH₂)—NH(CH₂CH₂O)₃—(CH₂)₂—NH—,
—(CH₂)—NH(CH₂CH₂O)₃—(CH₂)₃—NCH₃—,
—(CH₂)—NH(CH₂CH₂O)₄—(CH₂)₃—NCH₃—,
—(CH₂)—NH(CH₂CH₂O)₃—,
—(CH₂)—NH(CH₂CH₂O)₄—,
—(CH₂)—NH(CH₂CH₂O)₃—(CH₂)₂—NH—C(O)CH₂—O—,
—(CH₂)—NH(CH₂CH₂O)₃—(CH₂)₂—,
—(CH₂)—NH(CH₂CH₂O)₃—(CH₂)₃—,

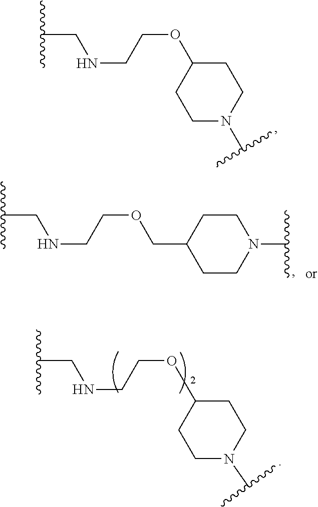

In some embodiments of the compound of formula (X-III), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, L² is:

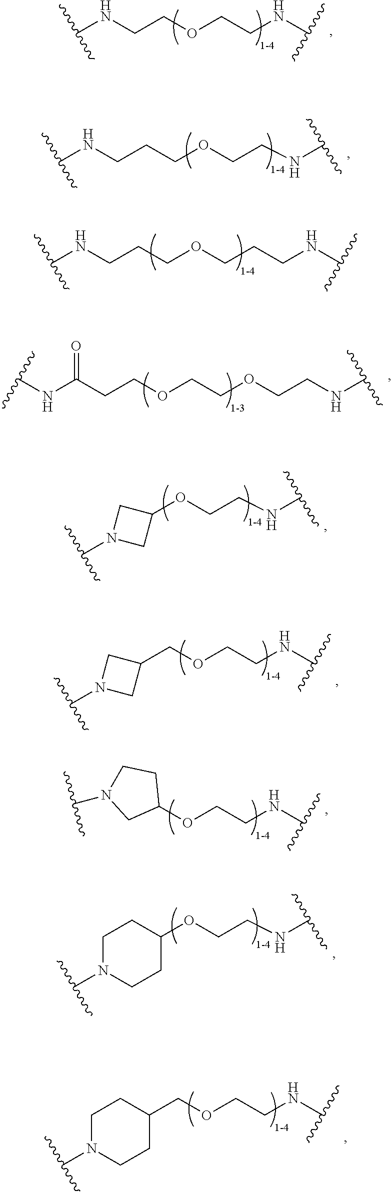

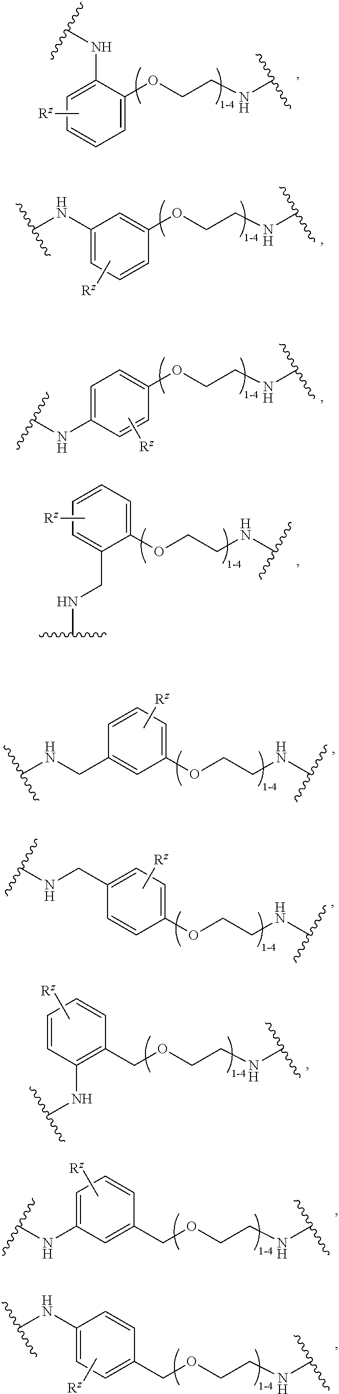

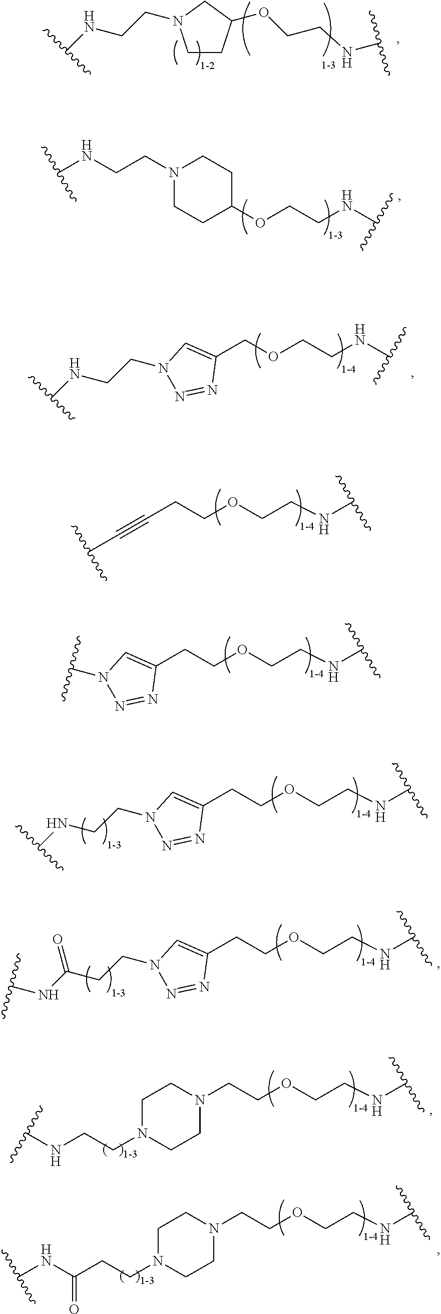

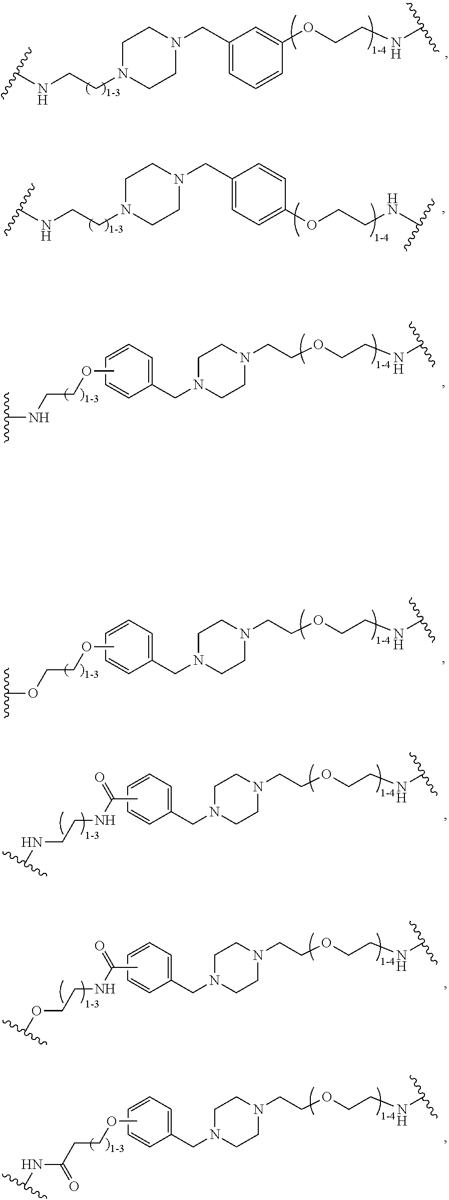

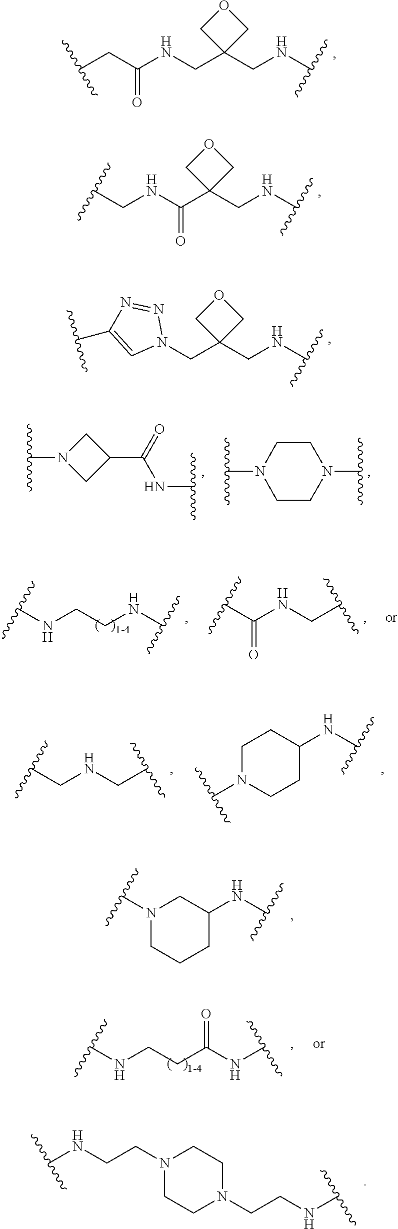

In embodiments of the compound of formula (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), or (II-G-1), or a pharmaceutically acceptable salt, stereoisomer, or deuterated form thereof, wherein L¹ comprises a bivalent moiety selected from:

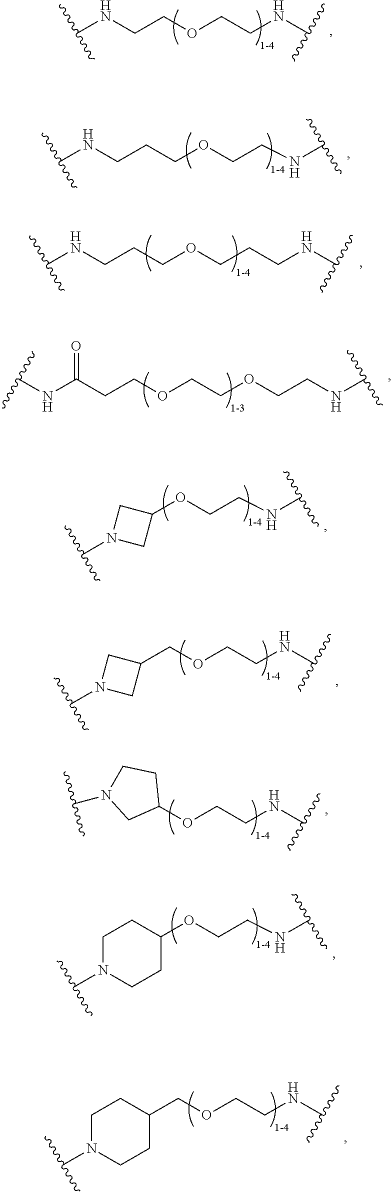

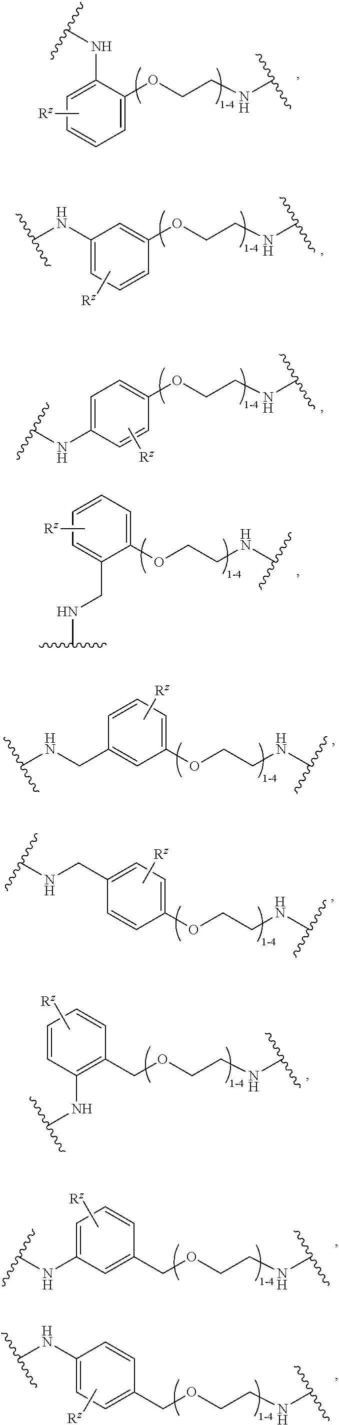

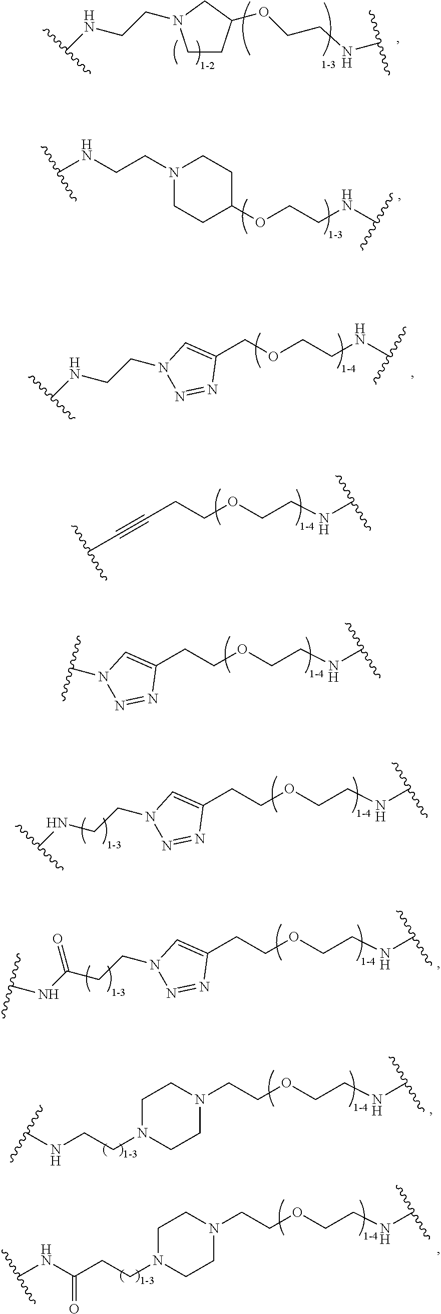

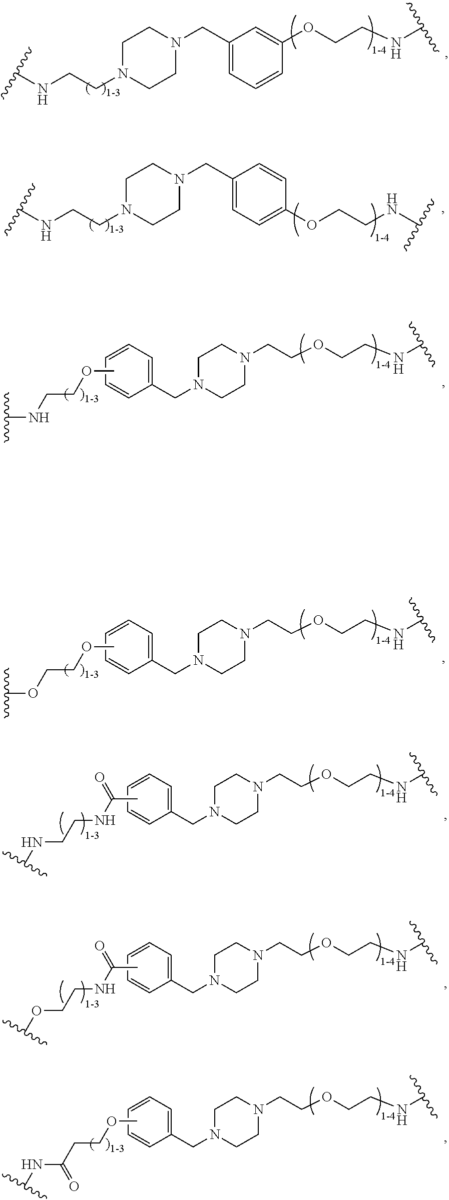

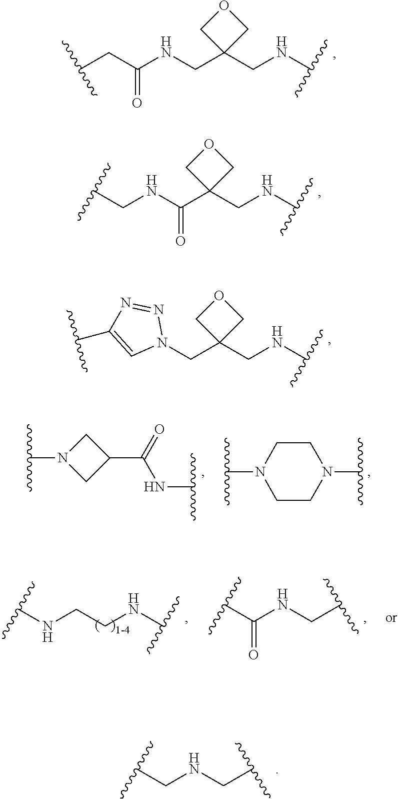

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, other linker moieties can be used as L² to covalently link A with ring W. Non-limiting examples of additional linker moiety as L² include the following:
(i) -Q1-(CH₂CH₂O)ₘ₁—(CH₂)ₙ₁—P1-, -Q1-(CH₂CH₂CH₂O)ₘ₁—(CH₂)ₙ₁—P1-, -Q1-(CH₂CH₂NH)ₘ₁—(CH₂)ₙ₁—P1-, and -Q1-(CH₂CH₂CONH)ₘ₁—(CH₂)ₙ₁—P1-, wherein Q1 is —NH—, —O—, ═N—, or —N(CH₃)—, which is a portion modified by binding to the moiety A; P1 is —NH—, —O—, —CH₂—, —C(═O)—; m1 is an integer of 0 to 4; and n1 is an integer of 0 to 3;
(ii) —(CH₂)ₙ₂—NH—C(═O)—, —(CH₂)ₙ₃—NH—C(═O)—(CH₂)ₙ₄—O—, —CH₂—CH₂—O—, —CH₂—C(═O)—, wherein n₂, n₃, and n₄ are independently an integer of 1 to 7;

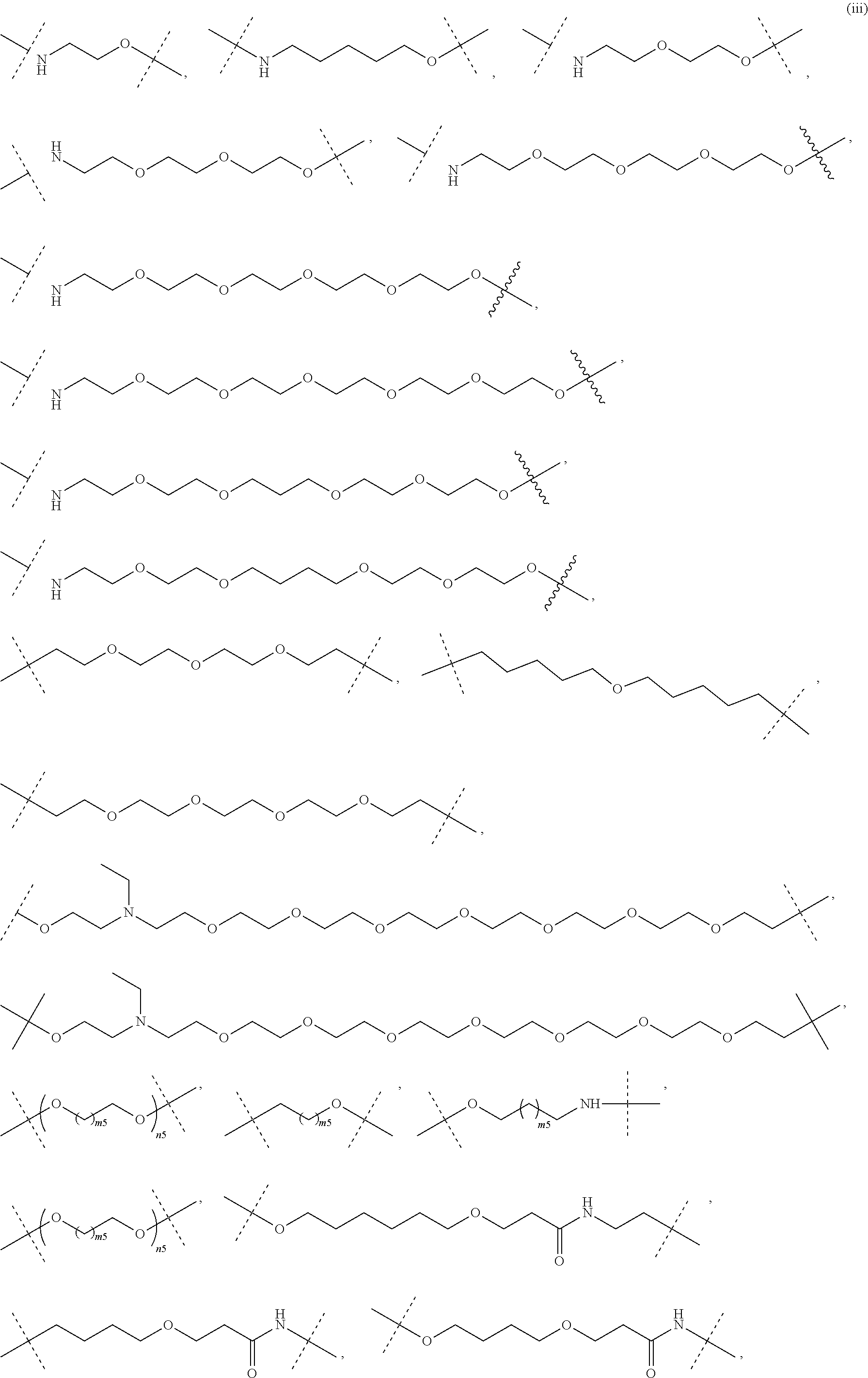

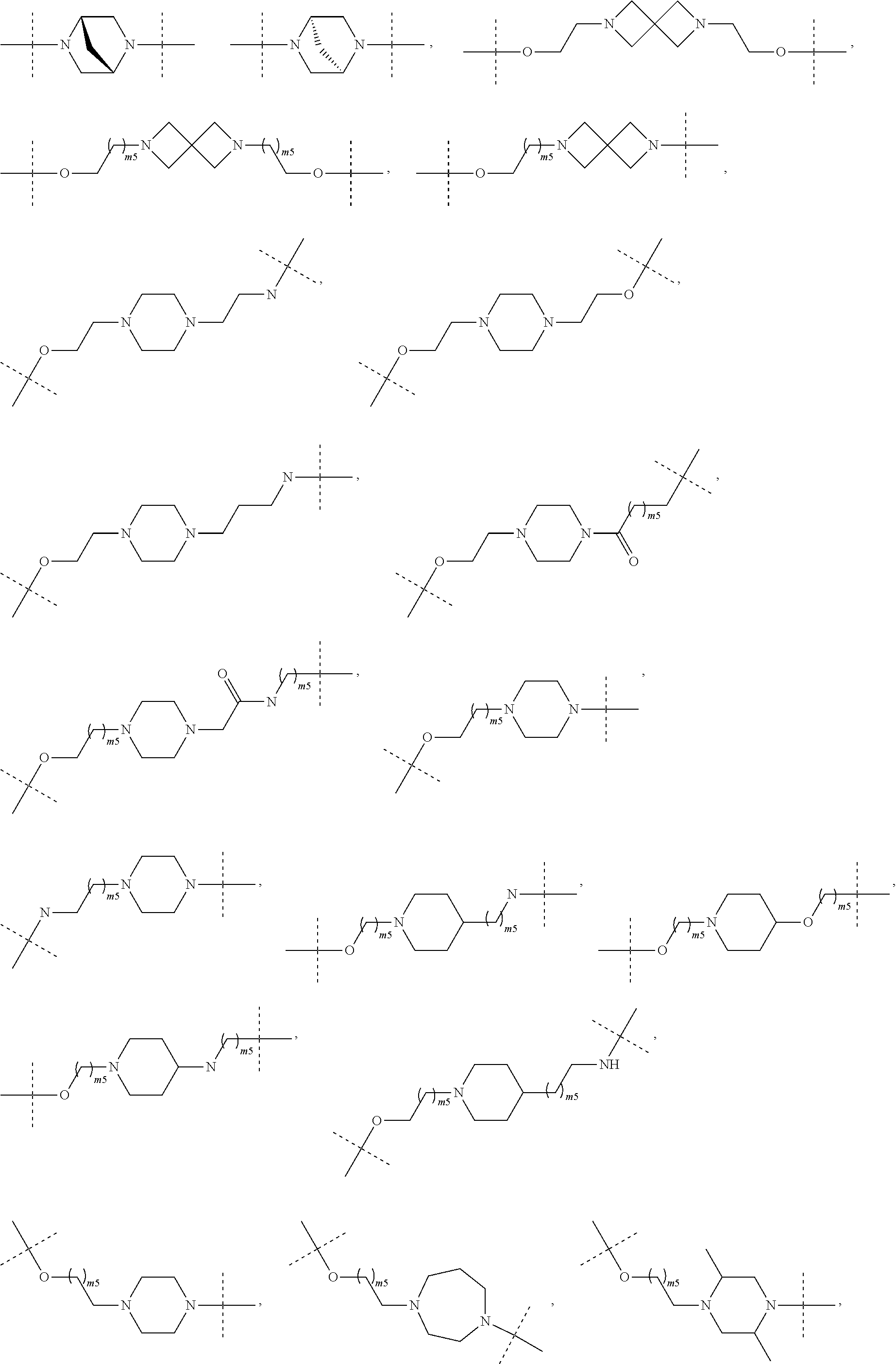

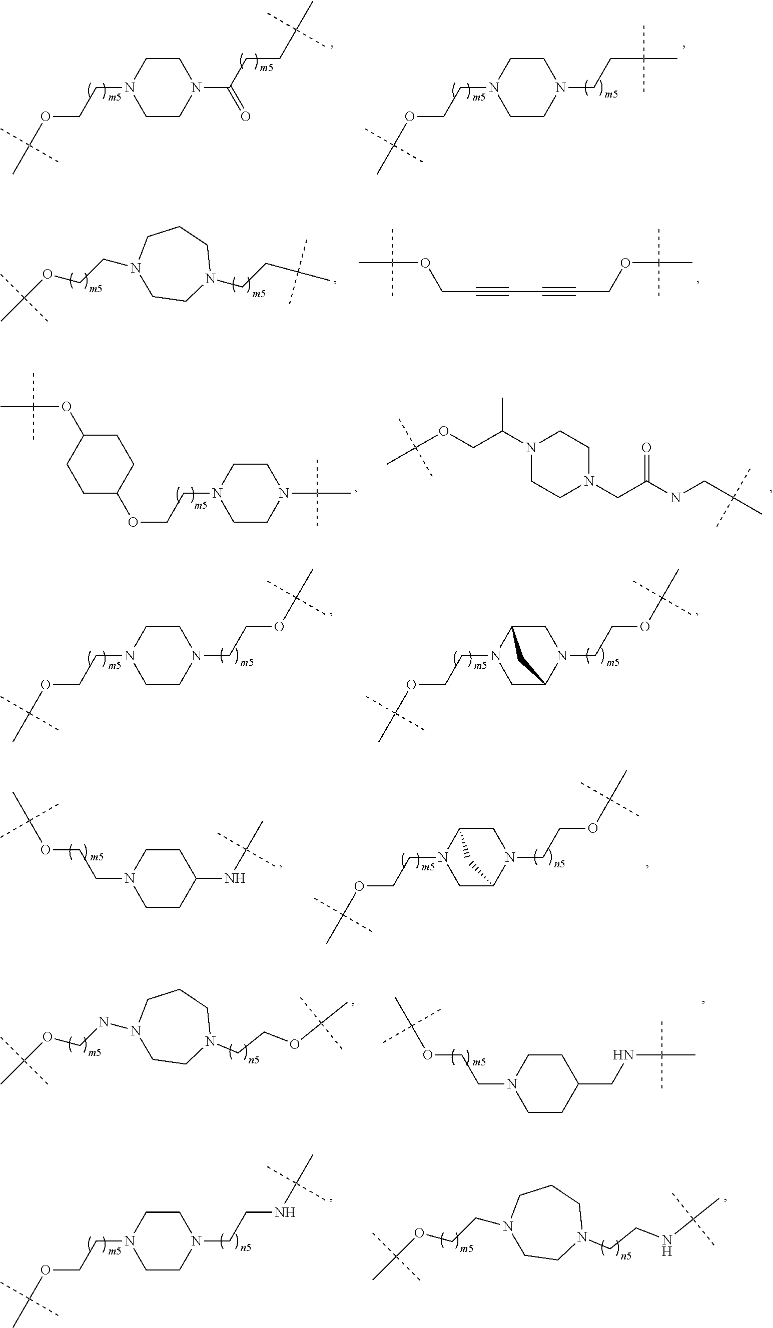

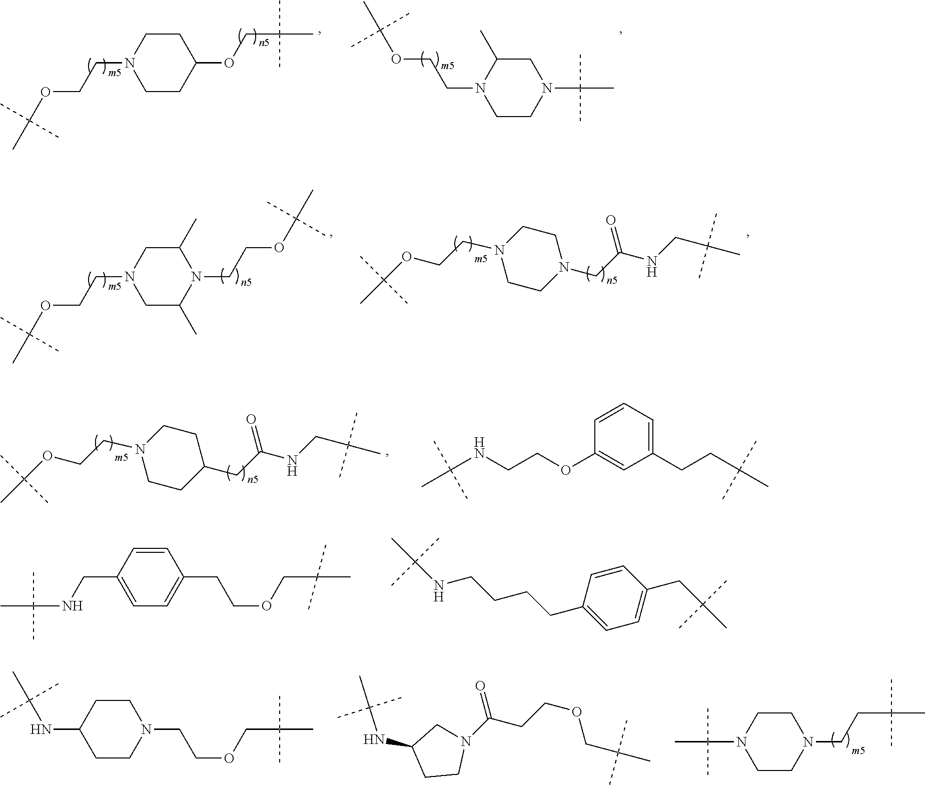

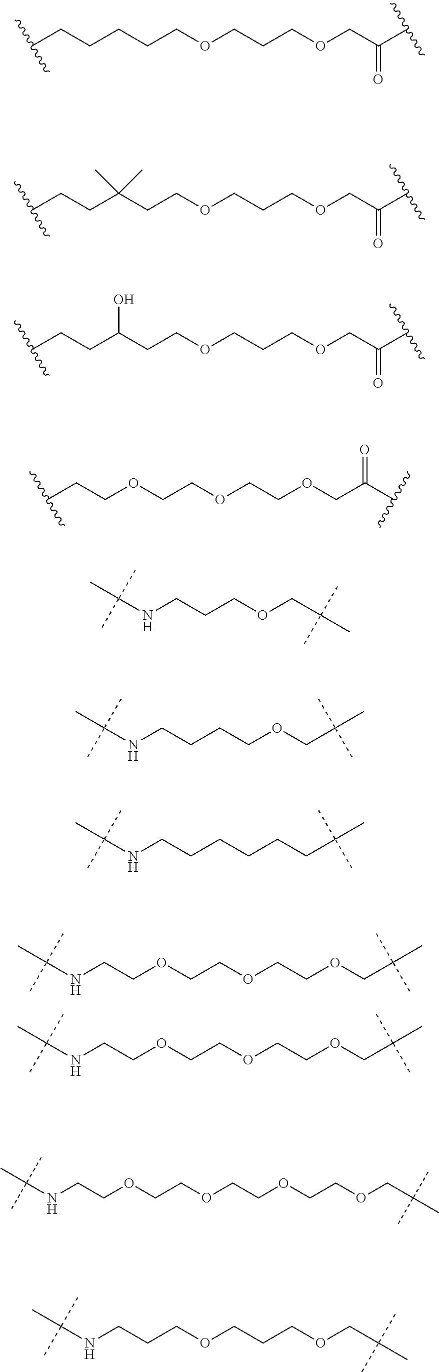

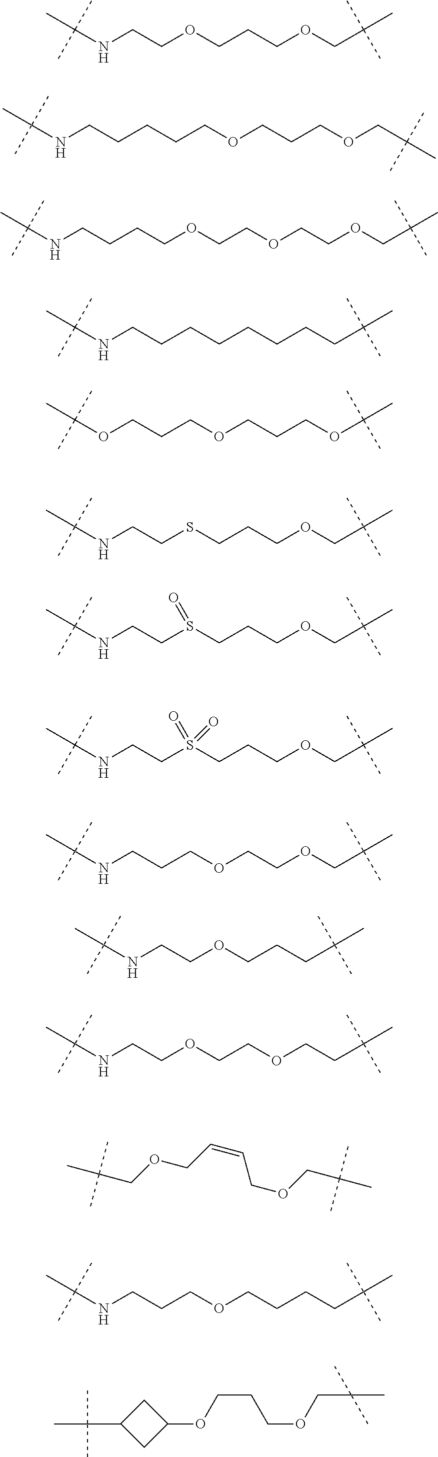

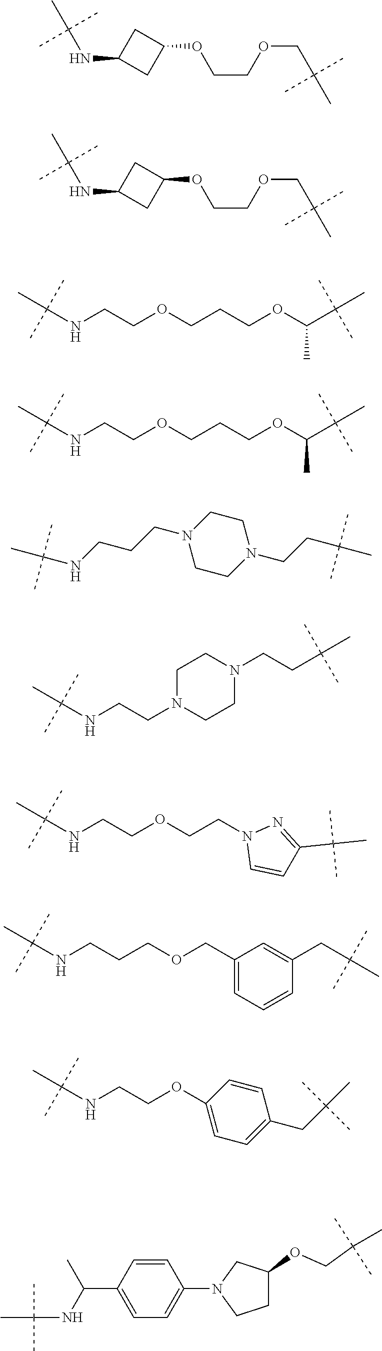

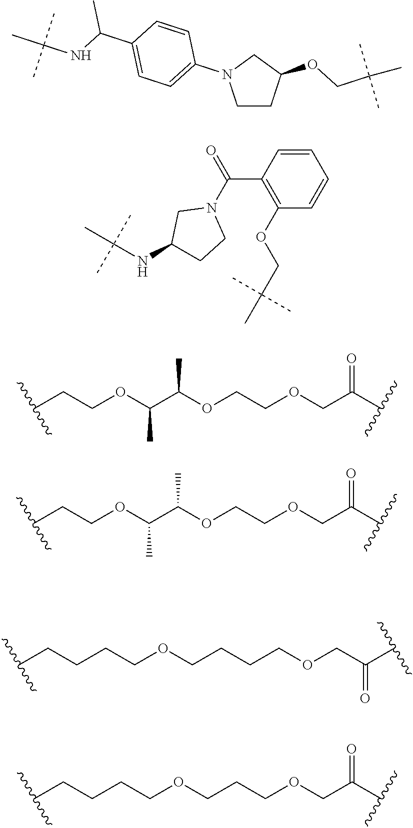

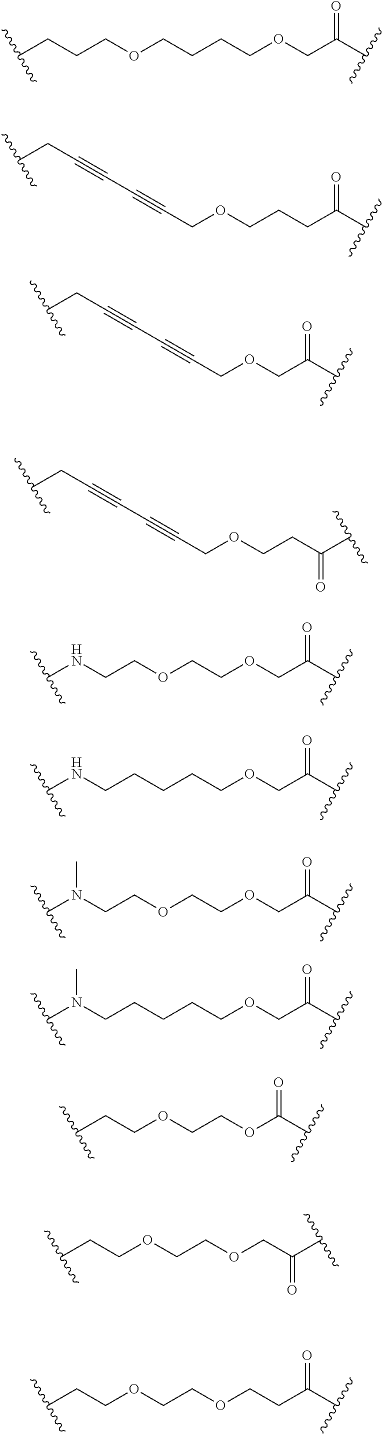

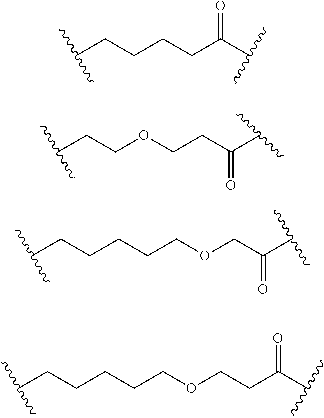

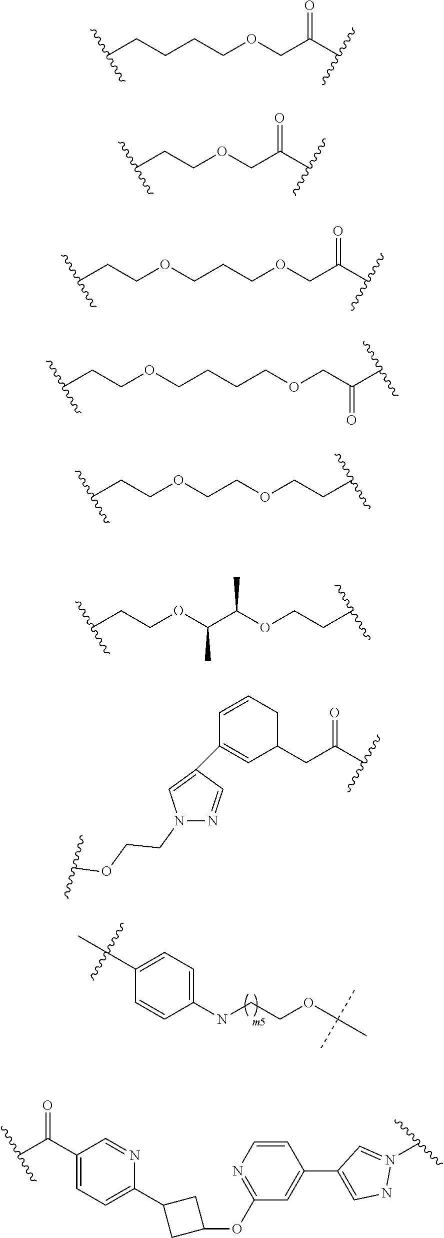

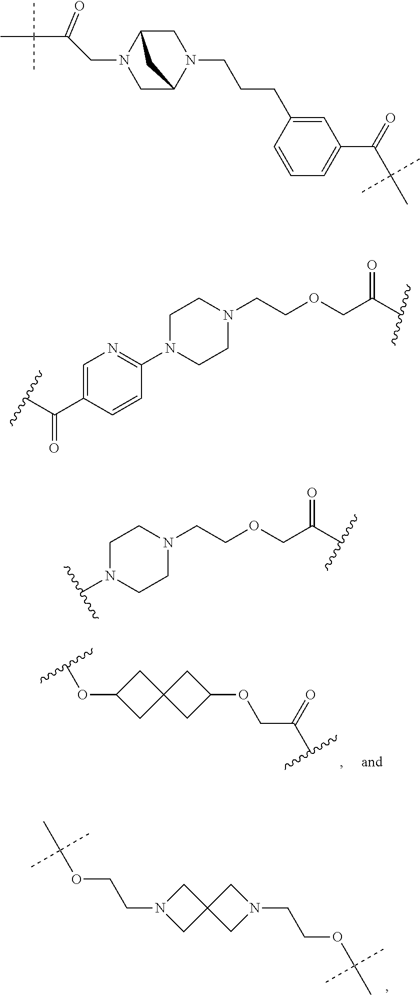

wherein m5 and n5 are each independently an integer of 0-6;
(iv) —(CH₂)ₘ₆—(OCH₂CH₂)ₙ₆O(CH₂)ₘ₆—, —O—(CH₂)ₘ₆—(OCH₂CH₂)ₘ₆O—(CH₂)ₘ₆O—, —NH—(CH₂)ₘ₆—(OCH₂CH₂)ₘ₆O—(CH₂)ₘ₆O—, —O—(CH₂)ₘ₆—(OCH₂CH₂)O—(CH₂)ₘ₆NH—, —O—(CH₂)ₘ₆—NH—(CH₂)ₘ₆—(OCH₂CH₂)ₘ₆O—(CH₂)ₘ₆—, —O—(CH₂)ₘ₆—NCH₃—(CH₂)ₘ₆—(OCH₂CH₂)ₙ₆O—(CH₂)ₘ₆—, —O—(CH₂)ₘ₆—NCH₂CH₃—(CH₂)ₘ₆—(OCH₂CH₂)ₙ₆O—(CH₂)ₚ₆—, —(CH₂)ₘ₆—NH—(CH₂)ₘ₆—(OCH₂CH₂)ₙ₆O—(CH₂)ₚ₆—, —(CH₂)ₘ₆—NCH₃—(CH₂)ₘ₆—(OCH₂CH₂)ₙ₆O—(CH₂)ₚ₆—, and —(CH₂)ₘ₆—NCH₂CH₃—(CH₂)ₘ₆—(OH₂CH₂)ₙ₆—(CH₂)ₚ₆—,
wherein each m6 is independently an integer of 0-5, each n6 is independently an integer of 0-7, and each p6 is independently an integer of 0-2.

### Ligand A (Protein Binding Component “PBC”)

In some embodiments of the compound of formula (X), (III), (III-A), (III-B), (III-C), (III-D), (III-E), (III-F), (III-G), (III-A-1), (III-A-2), (II-A-3), (III-B-1), (III-C-1), (III-D-1), (III-E-1), (III-F-1), or (III-G-1), or a pharmaceutically acceptable salt, a stereoisomer, or a deuterated form thereof, A is a ligand (e.g., a PBC) that binds to a protein. Once a protein targeted for degradation is identified, any ligand that binds to said target protein may be used in the compounds of (X), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1) described herein. Such ligands may be covalently bound to the linker using a functional group found on the ligand, or the ligand may be modified to include an appropriate functional group to facilitate conjugation to the linker.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, the protein is a protein that is associated with cancer. In some embodiments, the protein associated with cancer comprises a mutation or a fusion. In some embodiments, the protein associated with cancer is BRD4. In some embodiments, the protein associated with cancer is BRD4, and A is

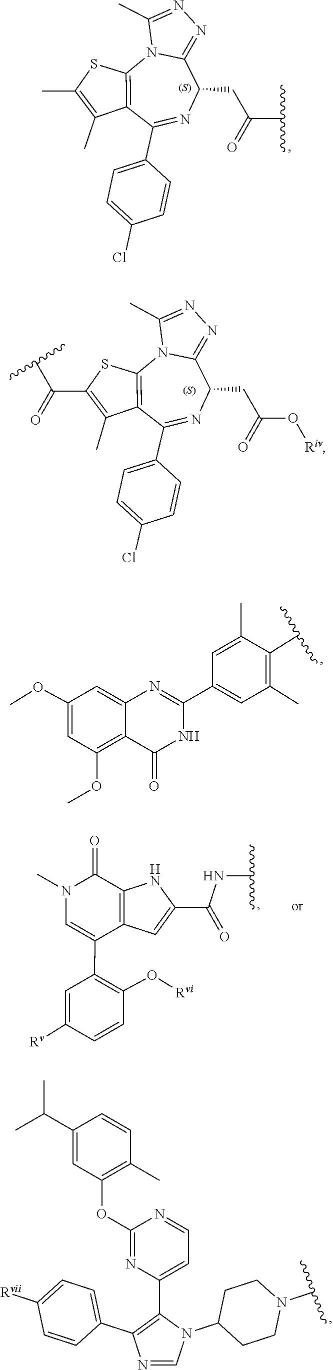

wherein:
R^{iv} is C₁₋₆ alkyl;
R^{v} is H,

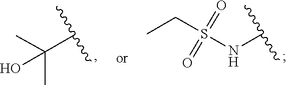

R^{vii} is

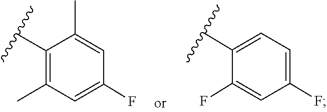

and
R^{vii} is H, F, CF₃, or C₁₋₆ alkyl.

In some embodiments, R^{iv} is methyl. Assays for assessing the binding of BRD4 with compounds having a corresponding ligand (e.g., the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof having the aforementioned A group) are generally known to a person of ordinary skill in the art. Exemplary BRD4 binding assays include, but are not limited to, surface plasmon resonance (SPR) assay, fluorescence polarization (FP), and thermal shift assay (TSA). See e.g., ACS Chem. Biol. 2019, 14, 3, 361-368; J Chem Inf Model. 2023, 63(17), 5408-5432; Structure 2023, 31(8), 912-923; SLAS Discovery 2015, 20(2), 180-189; Current Protocols in Pharmacology 2018, 80, 3.16.1-3.16.14, which are incorporated by reference herein in their entirety.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, the protein is a protein that is associated with a metabolic disease.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, the protein is a protein that is associated with inflammation.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, the protein is present in bacteria.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, the protein is present in a virus particle.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, A is a ligand that binds to a protein aggregate. In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, the protein aggregate is a Tau protein aggregate, an alpha-synuclein protein aggregate, a mutant Huntingtin protein aggregate, a β-sheet aggregate, a mitochondrial protein aggregate, an amyloid protein aggregate, or a TDP-43 protein aggregate. In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, the protein aggregate is an alpha-synuclein protein aggregate, a mutant Huntingtin protein aggregate, a β-sheet aggregate, an amyloid protein aggregate, or a TDP-43 protein aggregate.

In some embodiments, the protein aggregate is a Tau protein aggregate, and A is

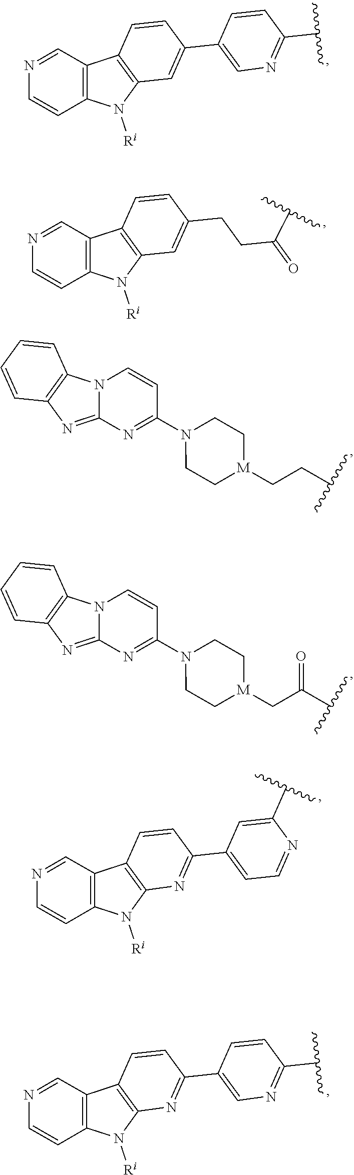

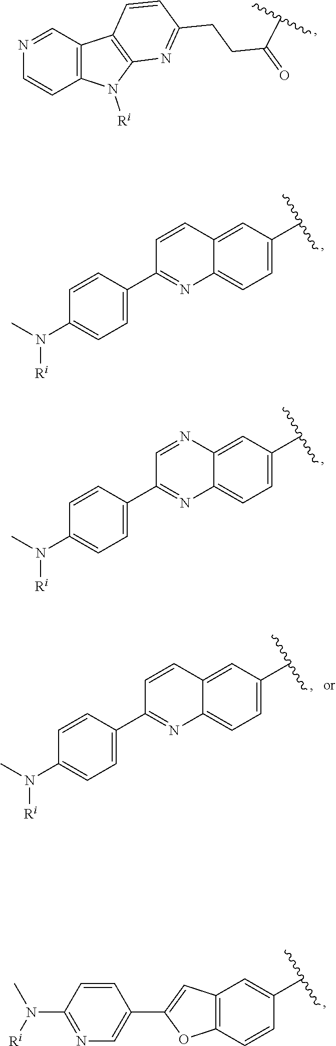

wherein: each Rⁱ is independently H or C₁₋₆ alkyl; and M is CH or N. In some embodiments, each Rⁱ is independently H or methyl.

Assays for assessing the binding of Tau with compounds having a corresponding ligand (e.g., the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof having the aforementioned A group) are generally known to a person of ordinary skill in the art. Exemplary Tau binding assays include, but are not limited to, radioligand binding assay, surface plasmon resonance (SPR) assay, differential scanning fluorimetry (DSF) and nuclear magnetic resonance (NMR) titration. See e.g., Chembiochem. 2023 May 16; 24(10):e202300163; J Chem Inf Model. 2023, 63(17), 5408-5432; Alzheimer's Res Therapy 2017, 9, 96; Eur J Nucl Med Mol Imaging 2024, 51, 3960-3977; bioRxiv 2024.03.15.585148, which are incorporated by reference herein in their entirety.

In some embodiments, the protein aggregate is an alpha-synuclein protein aggregate, and A is:

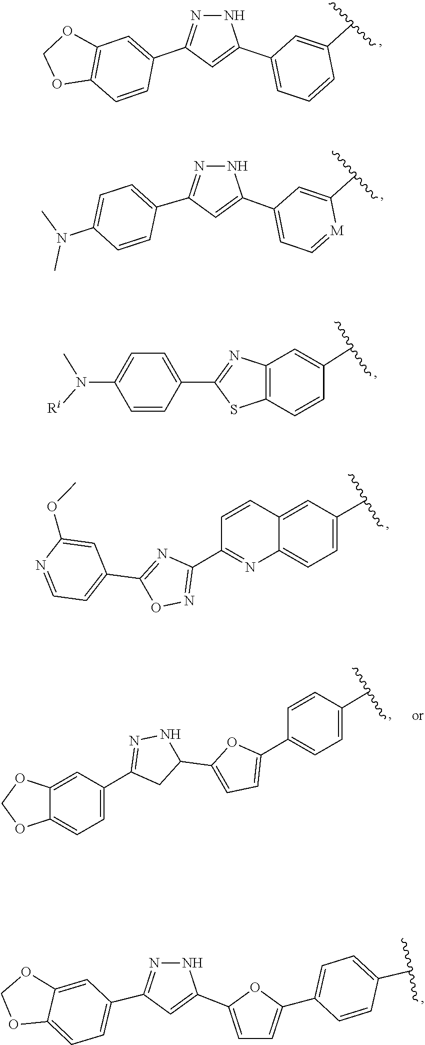

wherein: Rⁱ is H or C₁₋₆ alkyl; and M is CH or N. In some embodiments, Rⁱ is H or methyl.

Assays for assessing the binding of alpha-synuclein with compounds having a corresponding ligand (e.g., the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof having the aforementioned A group) are generally known to a person of ordinary skill in the art. Exemplary alpha-synuclein binding assays include, but are not limited to, nuclear magnetic resonance (NMR) spectroscopy, surface plasmon resonance (SPR) assay, and radioligand binding assay. See e.g., Chembiochem. 2023 May 16; 24(10):e202300163; J Chem Inf Model. 2023, 63(17), 5408-5432; Commun Biol 2018, 1, 44; J Neurochem. 2008, 105(4), 1428-37; Eur J Nucl Med Mol Imaging 2024, 51, 3960-3977, which are incorporated by reference herein in their entirety.

In some embodiments, the protein aggregate is a mutant Huntingtin protein aggregate (mHTT), and A is

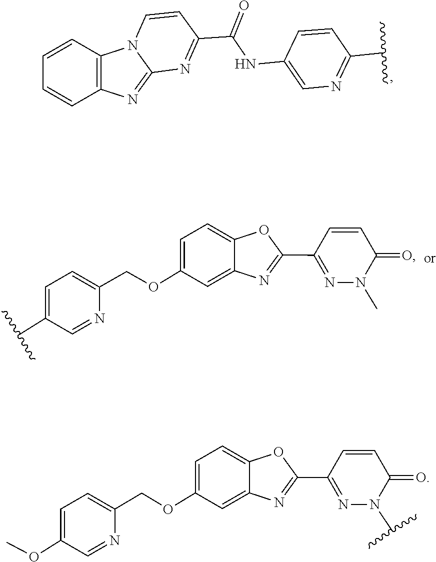 [SMILES: *c1ccc(NC(=O)c2ccn3c(n2)nc2ccccc23)cn1 ; *c1ccc(COc2ccc3oc(-c4ccc(=O)n(C)n4)nc3c2)nc1 ; *n1nc(-c2nc3cc(OCc4ccc(OC)cn4)ccc3o2)ccc1=O]

Assays for assessing the binding of mutant Huntingtin protein with compounds having a corresponding ligand (e.g., the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof having the aforementioned A group) are generally known to a person of ordinary skill in the art. Exemplary mutant Huntingtin protein binding assays include, but are not limited to, surface plasmon resonance (SPR) assay, differential scanning fluorimetry (DSF), and radioligand binding assay. See e.g., Chembiochem. 2023 May 16; 24(10):e202300163; J Chem Inf Model. 2023, 63(17), 5408-5432; Structure 2023, 31(9), 1121-1131.e6; Sci Rep 2021, 11, 17977, which are incorporated by reference herein in their entirety.

In some embodiments, the protein aggregate is a β-sheet aggregate, and A is

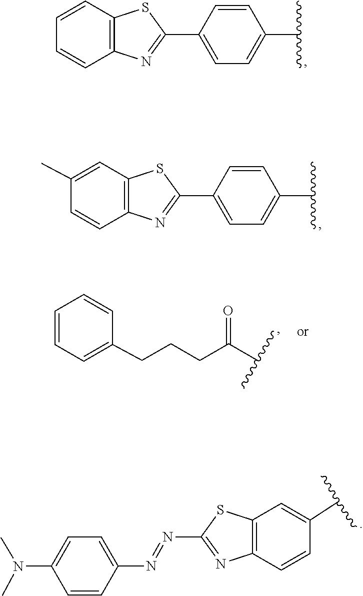 [SMILES: *c1ccc(-c2nc3ccccc3s2)cc1 ; *c1ccc(-c2nc3ccc(C)cc3s2)cc1 ; *C(=O)CCCc1ccccc1 ; *c1ccc2nc(/N=N/c3ccc(N(C)C)cc3)sc2c1]

Assays for assessing the binding of β-sheet aggregate with compounds having a corresponding ligand (e.g., the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof having the aforementioned A group) are generally known to a person of ordinary skill in the art. Exemplary β-sheet aggregate binding assays include, but are not limited to, fluorescence spectroscopy, surface plasmon resonance (SPR) assay, differential scanning fluorimetry (DSF), nanoscale differential scanning fluorimetry (nanoDSF), and thermal shift assay (TSA). See e.g., Chembiochem. 2023 May 16; 24(10):e202300163; J Chem Inf Model. 2023, 63(17), 5408-5432; Anal Biochem. 2022, 654, 114828; Eur J Nucl Med Mol Imaging 2024, 51, 3960-3977, which are incorporated by reference herein in their entirety.

In some embodiments, the protein is a mitochondrial protein, and A is

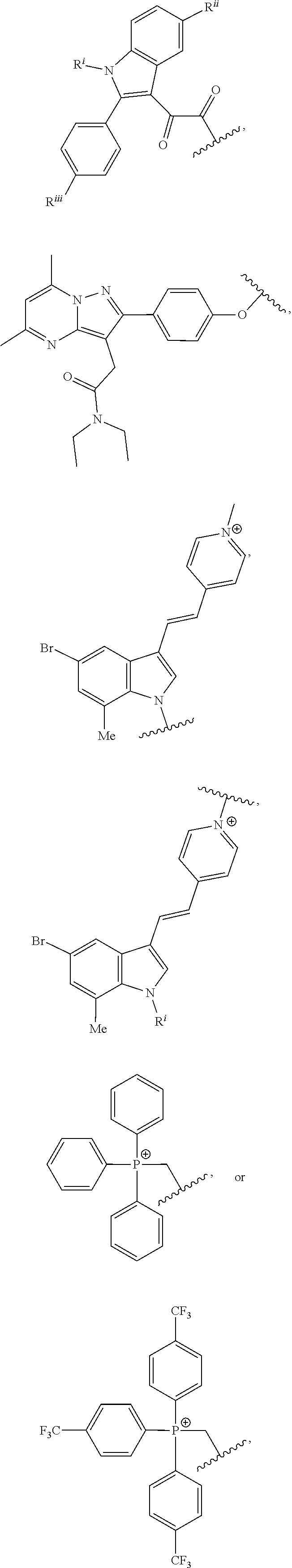

wherein: Rⁱ is H or C₁₋₆ alkyl, and Rⁱⁱ and Rⁱⁱⁱ are each independently a halogen or an alkyl. In some embodiments, Rⁱ is H or methyl, and Rⁱⁱ and Rⁱⁱⁱ are each independently F, Cl, or C₁₋₆ alkyl.

Assays for assessing the binding of mitochondrial protein with compounds having a corresponding ligand (e.g., the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof having the aforementioned A group) are generally known to a person of ordinary skill in the art. Exemplary mitochondrial protein binding assays include, but are not limited to, radioligand binding assay, surface plasmon resonance (SPR) assay, thermal shift assay (TSA), and X-ray crystallography. See e.g., Chembiochem. 2023 May 16; 24(10):e202300163; J Chem Inf Model. 2023, 63(17), 5408-5432; Journal of Med. Chem. 2004, 47(7), 1852-1855; Biochemistry 2023, 62, 7, 1262-1273, which are incorporated by reference herein in their entirety.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, the protein is an intracellular protein.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, A is aryl, arylene-heteroaryl, arylene, heteroarylene, aryl, heteroarylene-aryl, heteroarylene-heteroaryl, heteroarylene-heteroarylene-heteroaryl, heterocyclylene-aryl, heterocyclylene-heteroaryl, heteroarylene-NC(O)-heteroaryl, heteroarylene-N═N-aryl, or arylene-(arylalkyl)-OC(O)-hetercyclyl-C(O)C₁₋₆alkylene-aryl, each of which is optionally substituted with 1, 2, 3, or 4 groups independently selected from halogen, C₁₋₆ alkyl, O—C₁₋₆ alkyl, NH₂, NH(C₁₋₆ alkyl), N(C₁₋₆ alkyl)₂, —(CH₂)₁₋₄C(O)NH₂, —(CH₂)₁₋₄C(O)NH(C₁₋₆ alkyl), or —(CH₂)₁₋₄C(O)N(C₁₋₆ alkyl)₂.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, A is

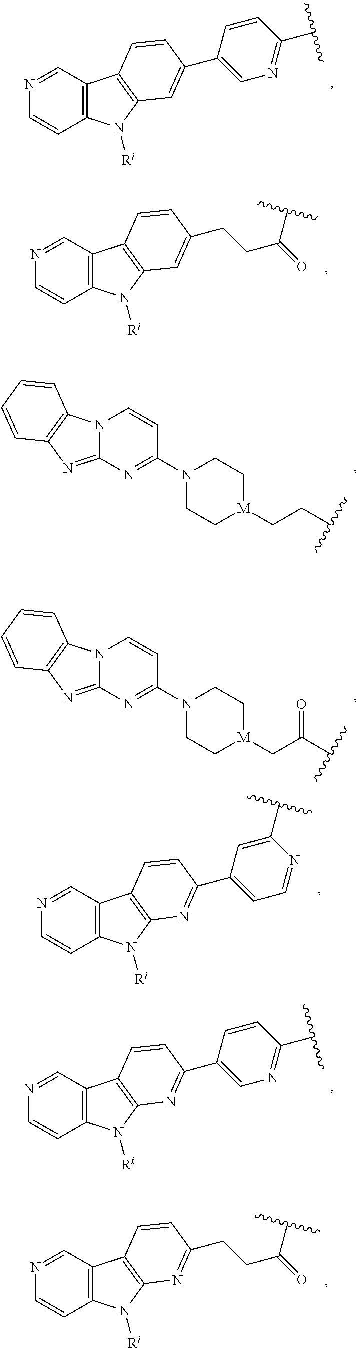

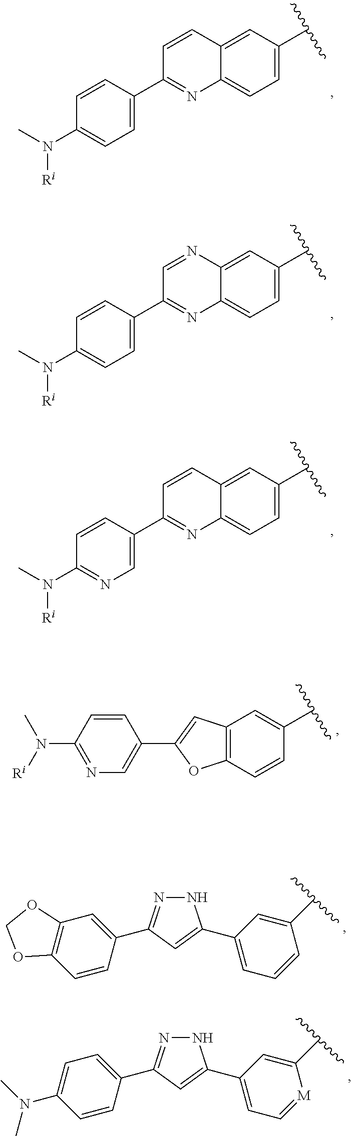

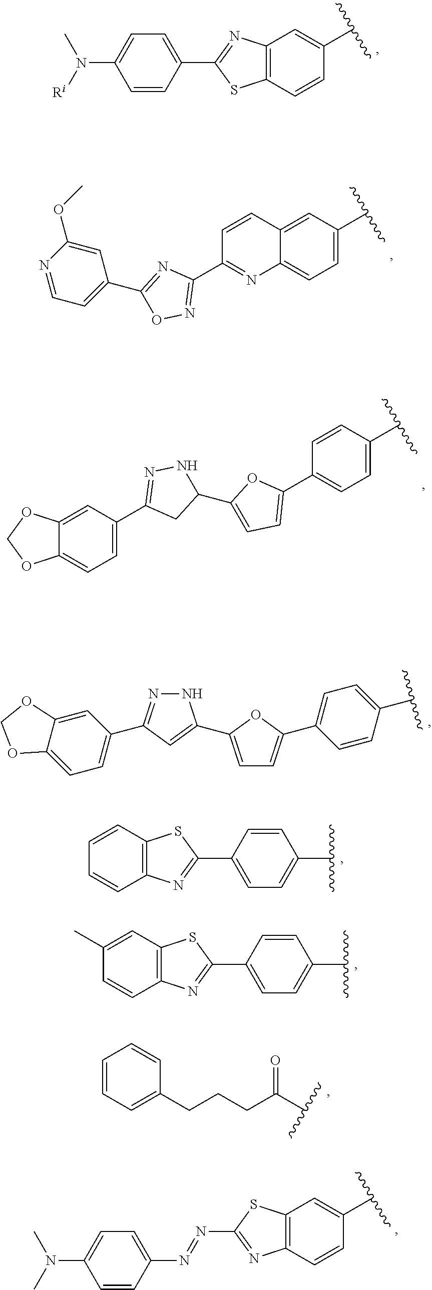

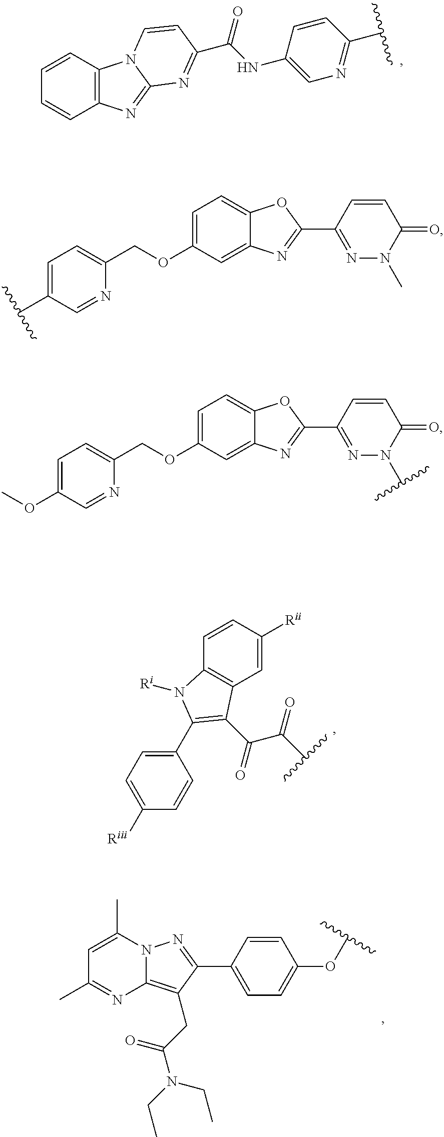

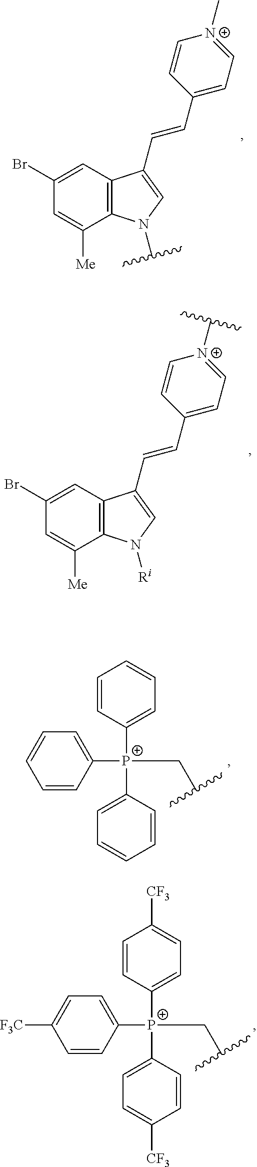

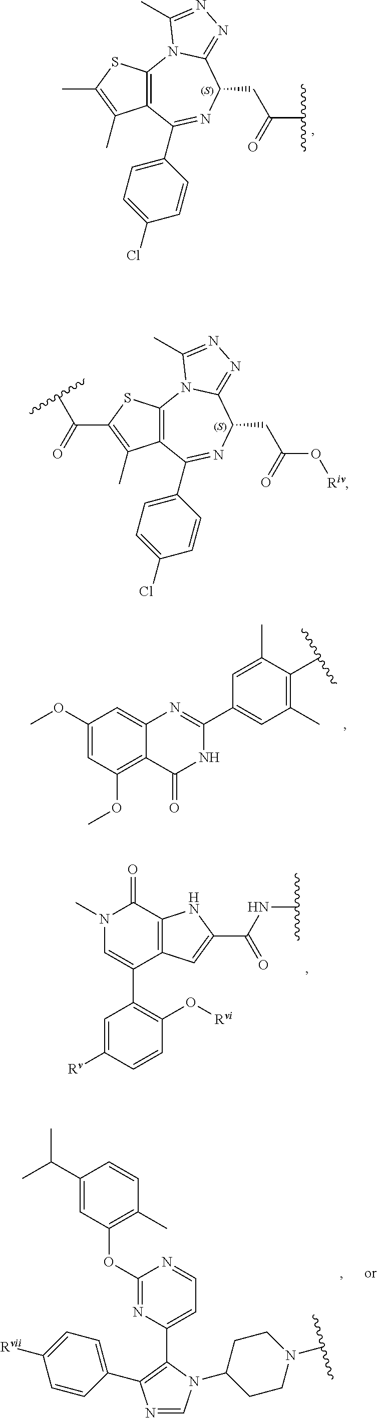

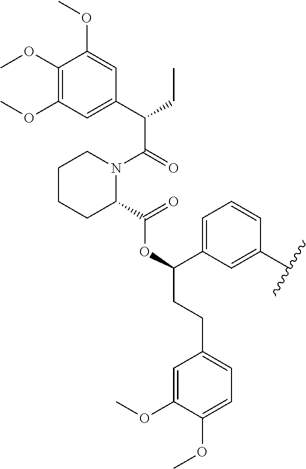

wherein:
each Rⁱ is independently H or C₁₋₆ alkyl;
Rⁱⁱ and Rⁱⁱⁱ are each independently F, Cl, or C₁₋₆ alkyl;
R^{iv} is C₁₋₆ alkyl;
R^{v} is H,

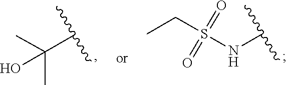

R^{vi} is

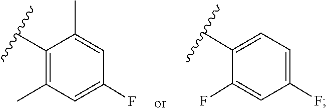

R^{vii} is H, F, CF₃, or C₁₋₆ alkyl; and
M is CH or N.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, A is

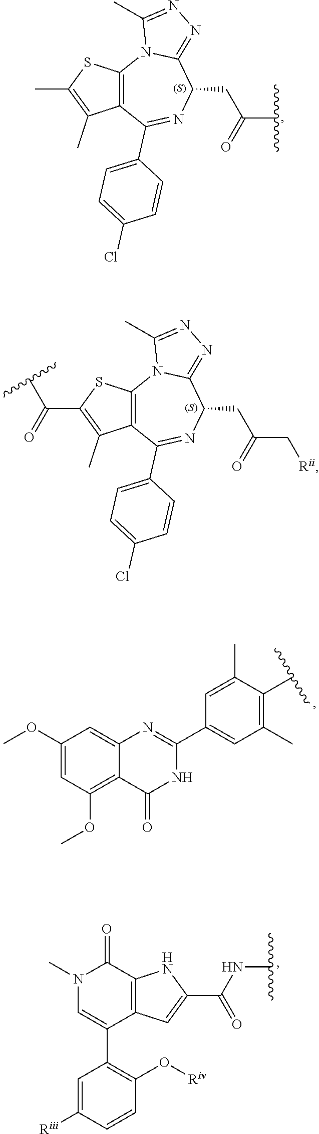

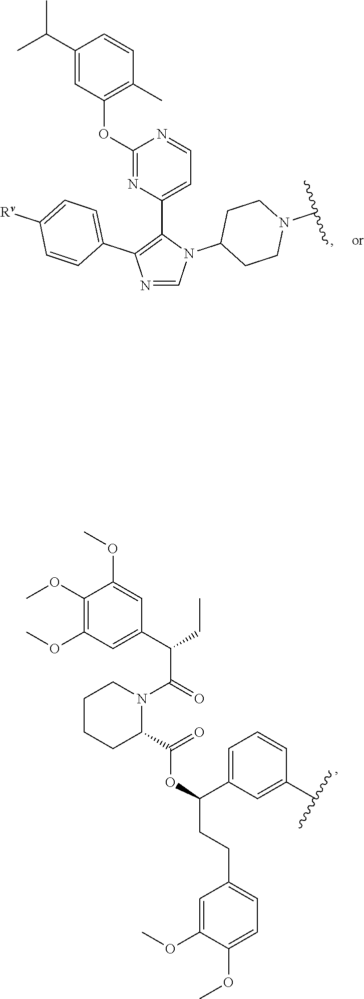

wherein:
Rⁱⁱ is C₁₋₆ alkyl;
Rⁱⁱⁱ is H,

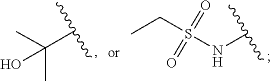

R^{iv} is

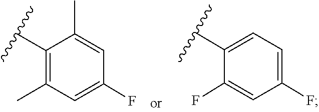

and
R^{v} is H, F, CF₃, or C₁₋₆ alkyl.

In some embodiments of the compound of formula (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, a stereoisomer, or a deuterated form thereof, other protein binding components (PBCs) that binds to a specific target protein, protein aggregates, protein complexes, lipids, or lipids can be used as ligand A. Non-limiting examples of the protein binding component include the following:

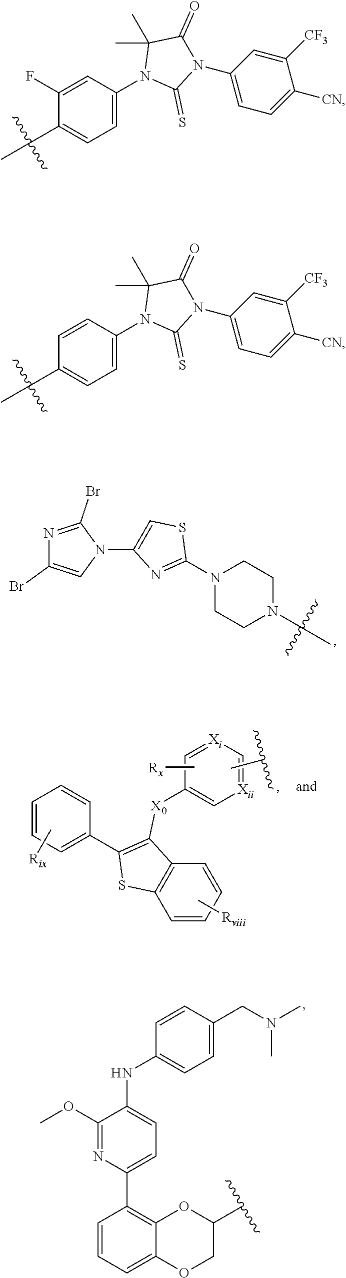

wherein:
X₀ is O or C═O;
Xᵢ and Xᵢᵢ are each independently N or CH;
Rᵥᵢᵢᵢ is OH, O(CO)Rᵢₓ, O—C₁₋₆ alkyl, wherein Rᵢₓ is an alkyl or aryl group;
Rᵢₓ is H, OH, halogen, CN, CF₃, SO₂—C₁₋₆ alkyl, O—C₁₋₆ alkyl; and
Rₓ is H or halogen.

In some embodiments, the protein binding component is a derivative (e.g., a monovalent derivative that covalently bonded to linker L²) of one of the following compounds:

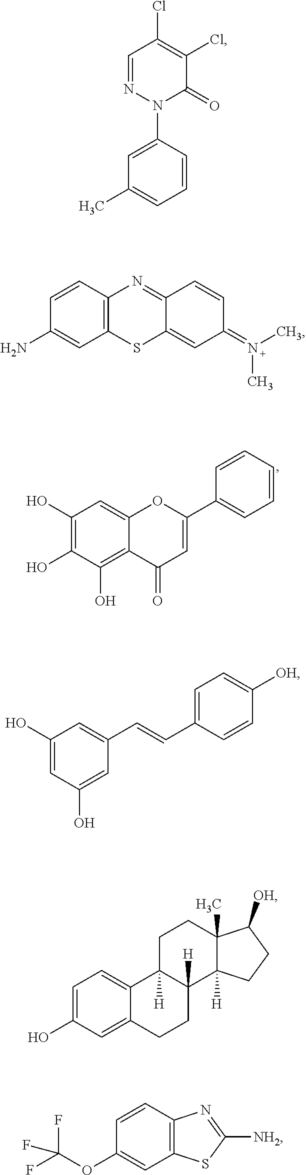

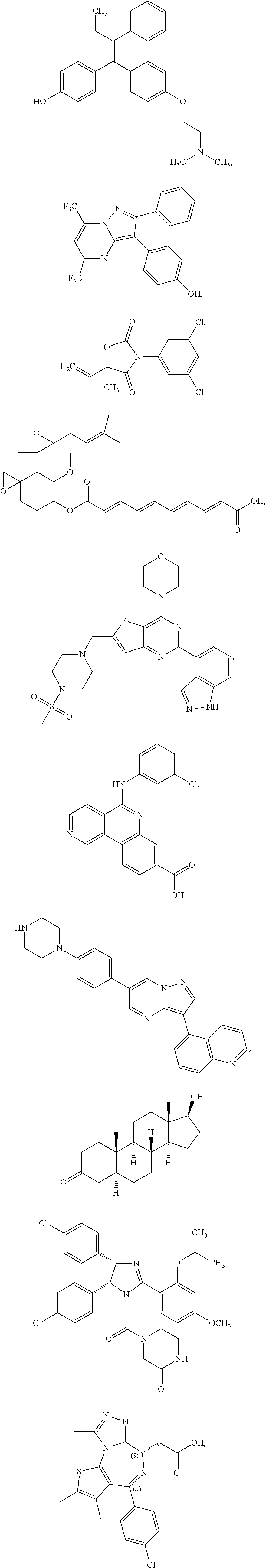

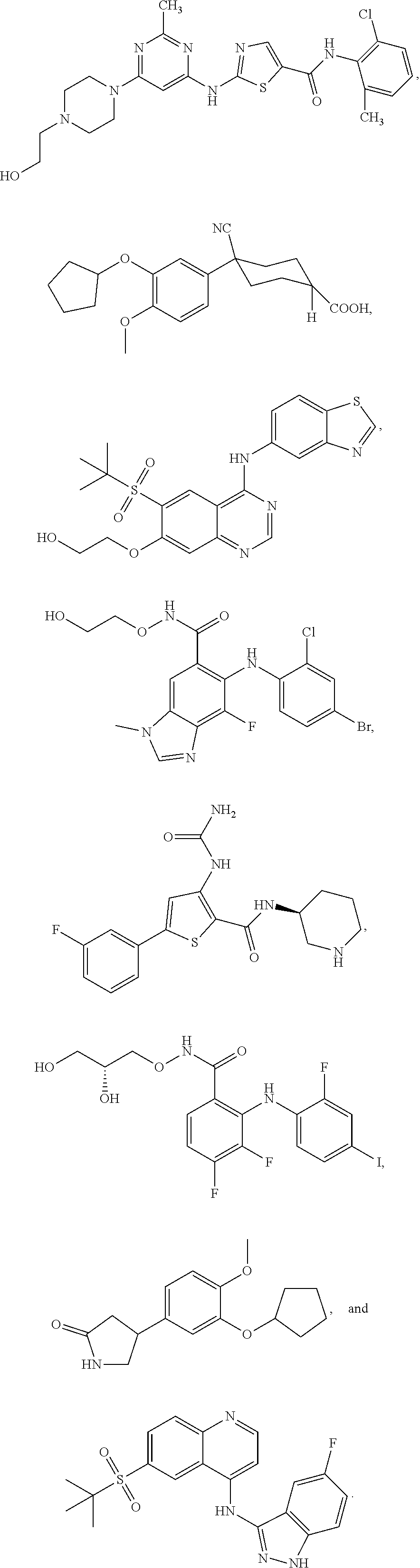

As a non-limiting example, a monovalent derivative of compound

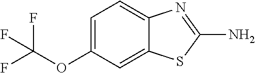 [SMILES: Nc1nc2ccc(OC(F)(F)F)cc2s1]

may be:

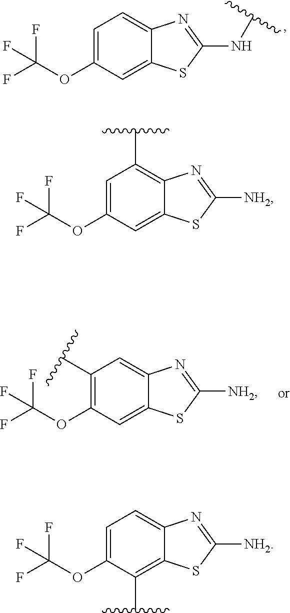 [SMILES: *Nc1nc2ccc(OC(F)(F)F)cc2s1 ; *c1cc(OC(F)(F)F)cc2sc(N)nc12 ; *c1cc2nc(N)sc2cc1OC(F)(F)F ; *c1c(OC(F)(F)F)ccc2nc(N)sc12]

In some embodiments, the protein binding component is an androgen receptor (AR) binding ligand. Non-limiting examples of androgen receptor binding ligand as protein binding component include:

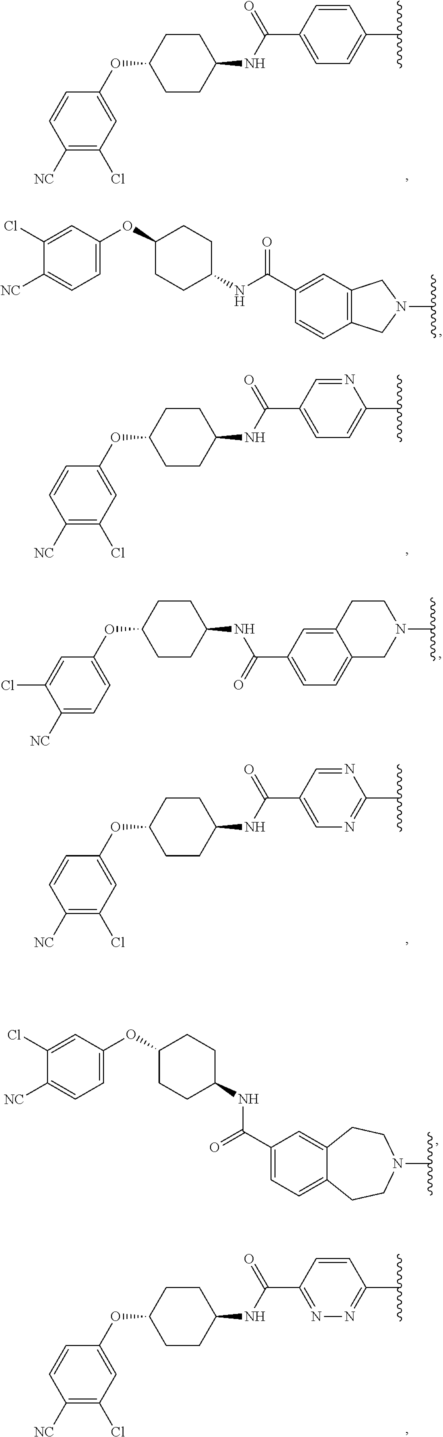

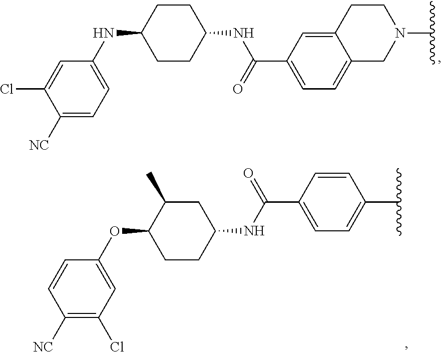 [SMILES: *N1CCc2cc(C(=O)N[C@H]3CC[C@H](Nc4ccc(C#N)c(Cl)c4)CC3)ccc2C1 ; *c1ccc(C(=O)N[C@@H]2CC[C@@H](Oc3ccc(C#N)c(Cl)c3)[C@@H](C)C2)cc1]

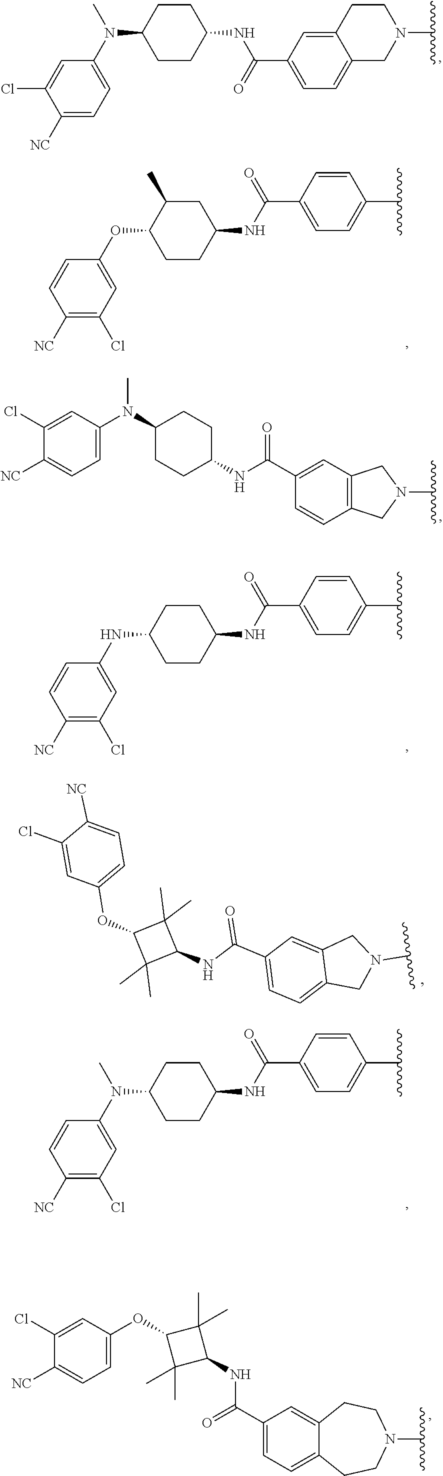

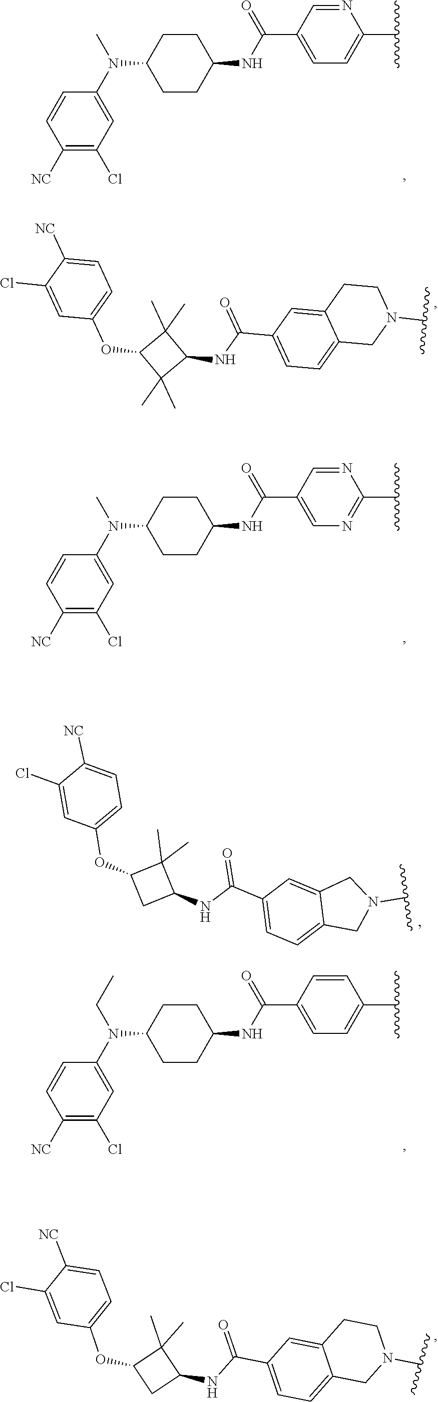

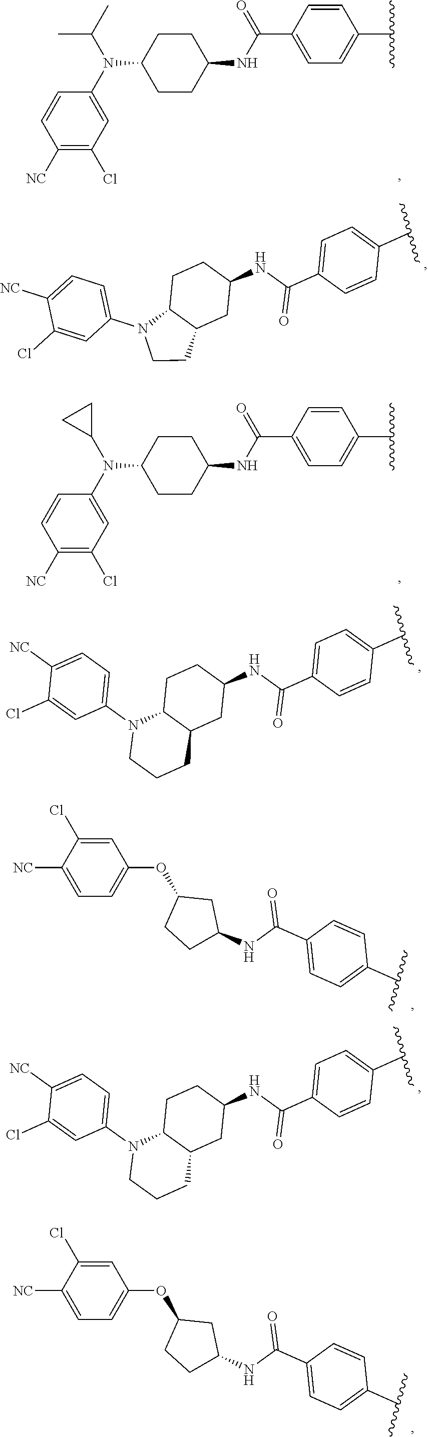

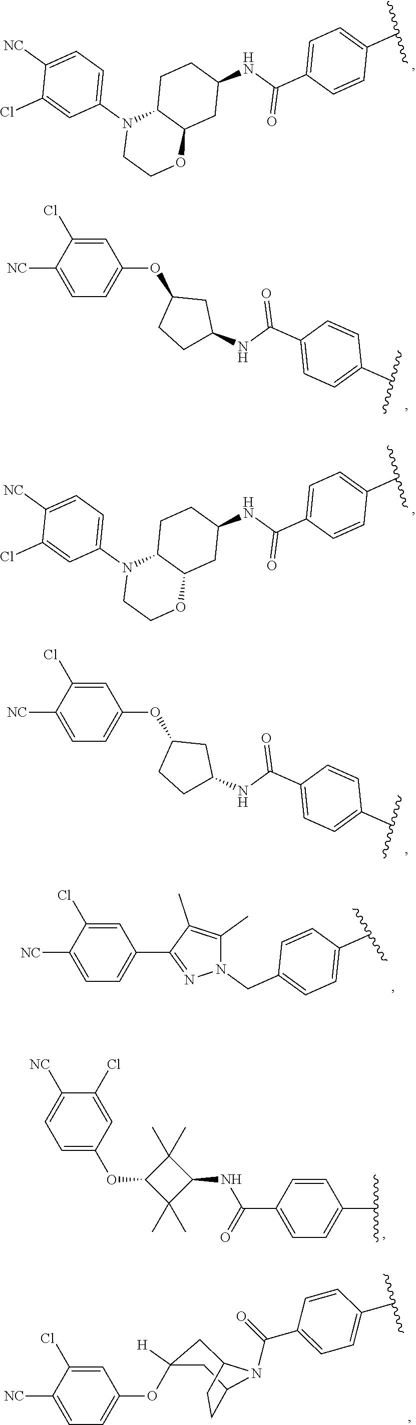

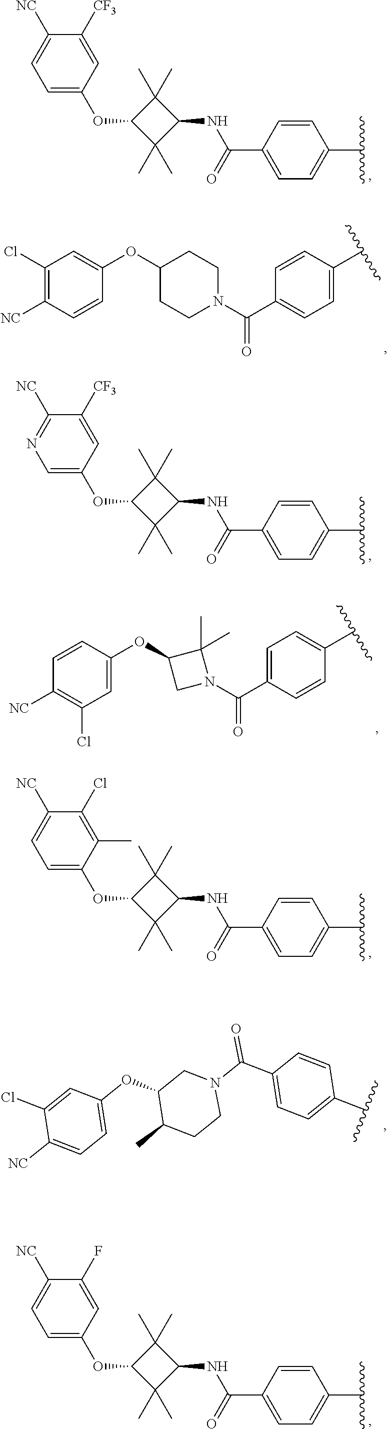

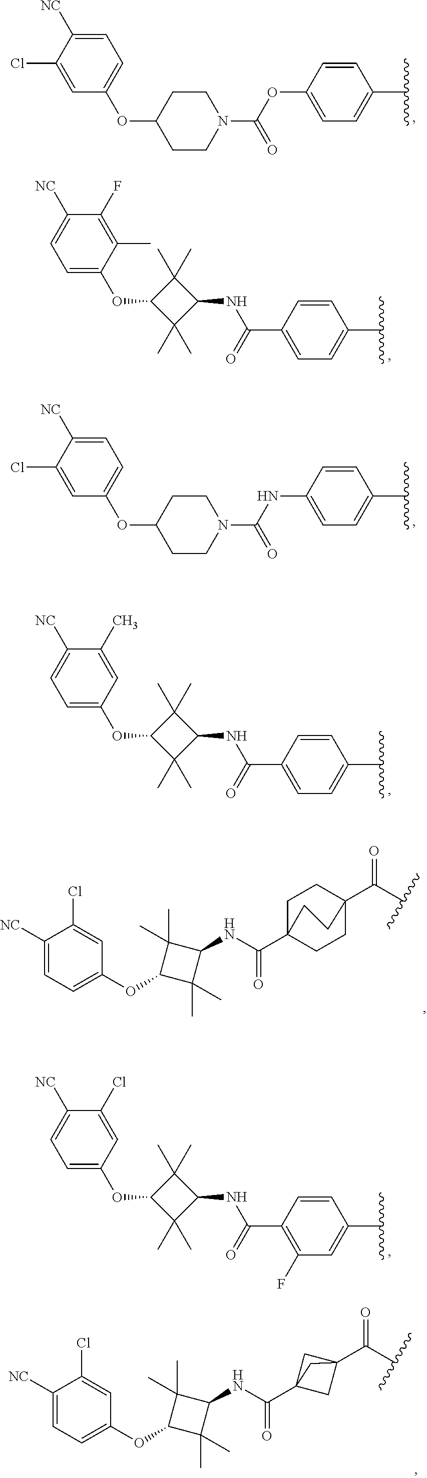

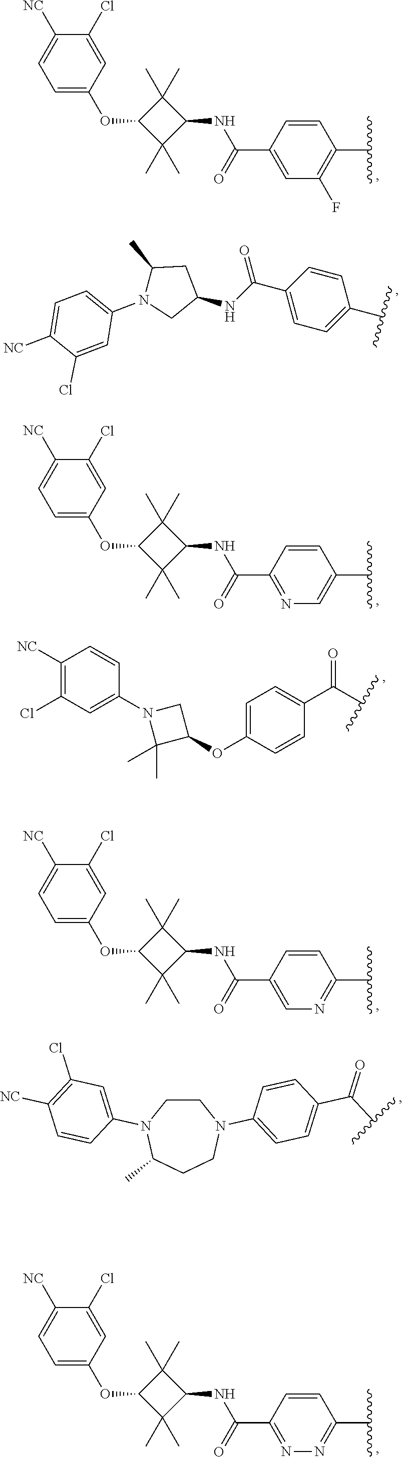

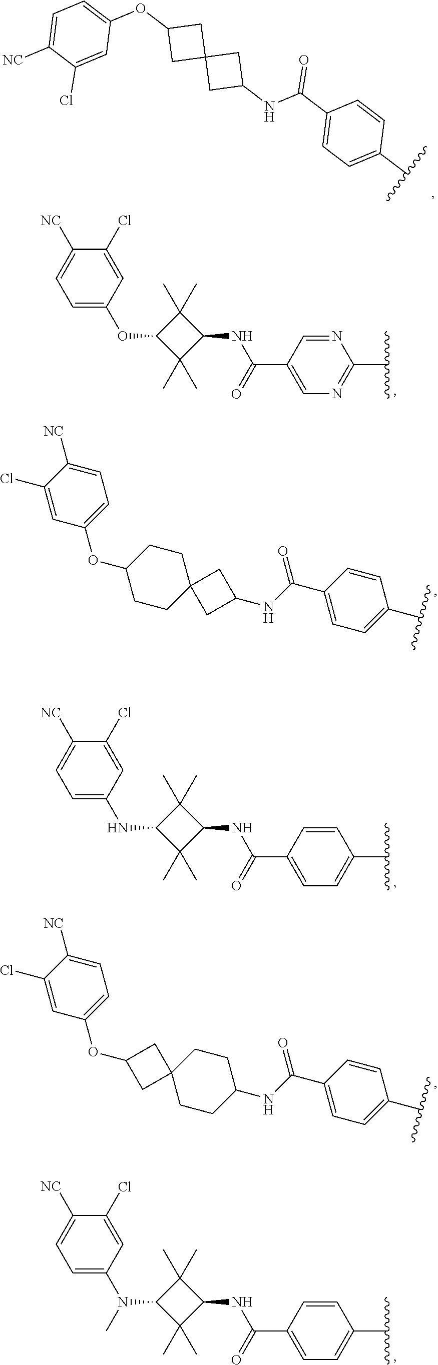 [SMILES: *c1ccc(C(=O)NC2CC3(C2)CC(Oc2ccc(C#N)c(Cl)c2)C3)cc1 ; *c1ncc(C(=O)N[C@H]2C(C)(C)[C@H](Oc3ccc(C#N)c(Cl)c3)C2(C)C)cn1 ; *c1ccc(C(=O)NC2CC3(CCC(Oc4ccc(C#N)c(Cl)c4)CC3)C2)cc1 ; *c1ccc(C(=O)N[C@H]2C(C)(C)[C@H](Nc3ccc(C#N)c(Cl)c3)C2(C)C)cc1 ; *c1ccc(C(=O)NC2CCC3(CC2)CC(Oc2ccc(C#N)c(Cl)c2)C3)cc1 ; *c1ccc(C(=O)N[C@H]2C(C)(C)[C@H](N(C)c3ccc(C#N)c(Cl)c3)C2(C)C)cc1]

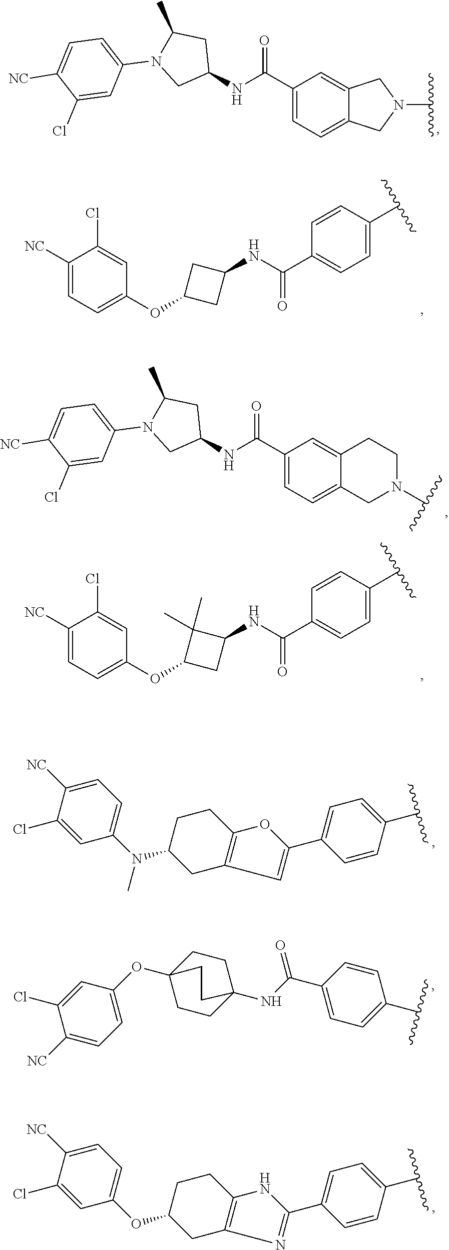

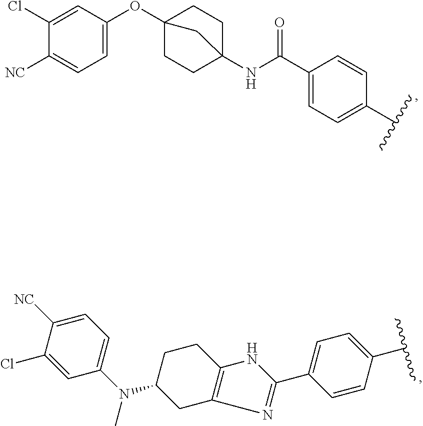 [SMILES: *c1ccc(C(=O)NC23CCC(Oc4ccc(C#N)c(Cl)c4)(CC2)C3)cc1 ; *c1ccc(-c2nc3c([nH]2)CC[C@@H](N(C)c2ccc(C#N)c(Cl)c2)C3)cc1]

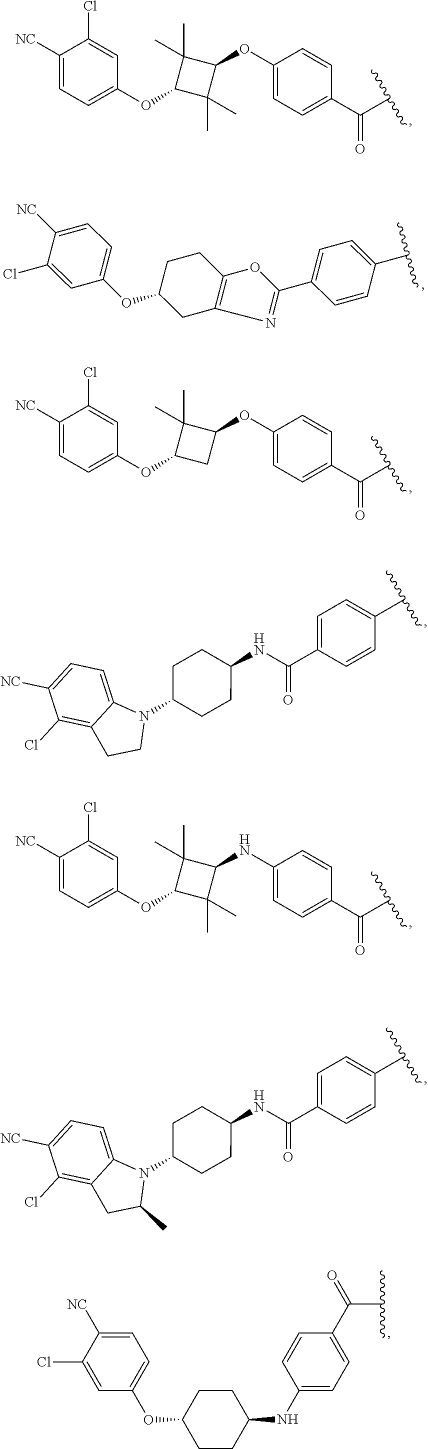

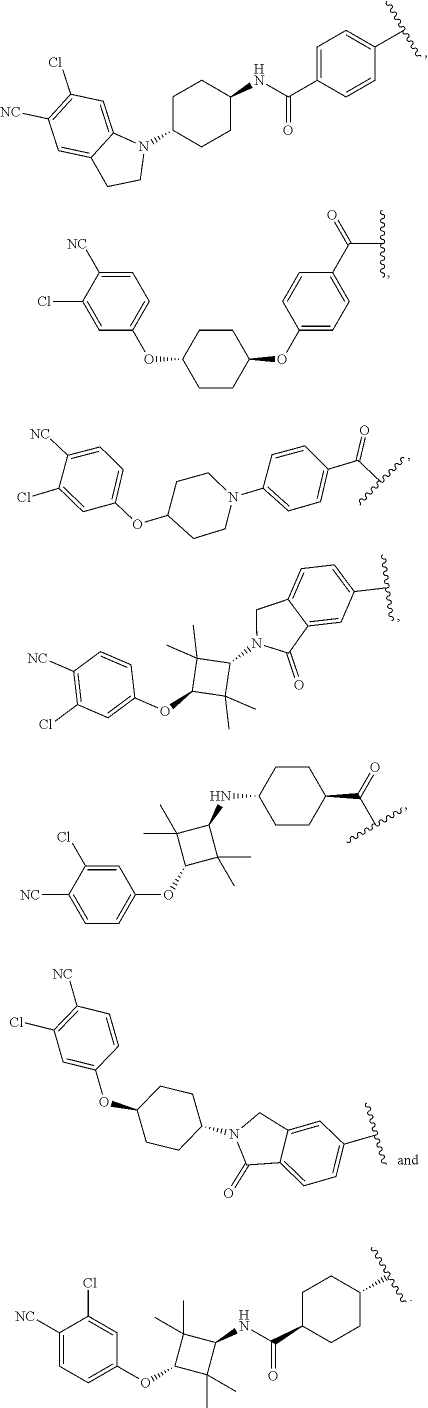

### R¹

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B3), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, each R¹ is independently optionally substituted C₁₋₆ alkyl, optionally substituted C₃-C₈ cycloalkyl, or halogen. In some embodiments, each R¹ is independently optionally substituted C₁₋₆ alkyl, C₂₋₅ alkyl, or C₃₋₄ alkyl. In some embodiments, each R¹ is independently optionally substituted C₃-C₈ cycloalkyl, C₄-C₇ cycloalkyl, or C₅-C₆ cycloalkyl. In some embodiments, each R¹ is independent halogen.

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B3), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, each R¹ is —F, —Cl, —Br, or —I. In some embodiments, each R¹ is —F or —Cl. In some embodiments, each R¹ is —F. In some embodiments, each R¹ is —Cl.

a

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B3), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, a is an integer of 0-3. In some embodiments, a is 0, 1, 2, or 3. In some embodiments, a is 1 or 2. In some embodiments, a is 0 or 1. In some embodiments, a is 0. In some embodiments, a is 1.

### R²

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, each R² is independently optionally substituted C₁₋₆ alkyl, optionally substituted C₃-C₈ cycloalkyl, or halogen, or two R² form an oxo. In some embodiments, each R² is independently optionally substituted C₁₋₆ alkyl, C₂₋₅ alkyl, or C₃₋₄ alkyl. In some embodiments, each R² is independently optionally substituted C₃-C₈ cycloalkyl, C₄-C₇ cycloalkyl, or C₅-C₆ cycloalkyl. In some embodiments, each R² is independently halogen. In some embodiments, two R² form an oxo. In some embodiments, each R² is independently C₁₋₆ alkyl, C₂₋₅ alkyl, or C₃₋₄ alkyl.

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, each R² is independently C₁₋₃ alkyl (e.g., methyl, ethyl, n-propyl, isopropyl).

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, each R² is methyl.

b

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, b is an integer of 0-2. In some embodiments, b is 0, 1, or 2. In some embodiments, b is 1 or 2. In some embodiments, b is 0 or 1. In some embodiments, b is 0. In some embodiments, b is 1.

### Q

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—, wherein the alkylene is optionally and independently substituted. In some embodiments, Q is a absent, C₁₋₄ alkylene, —(C₁₋₃ alkylene)C(O)—, C₃₋₆ cycloalkylene, —(C₃₋₆ cycloalkylene)C(O)—, —C(O)—, or —S(O)₂—, wherein the alkylene is optionally substituted. In some embodiments, Q is a absent,

In some embodiments, Q is C₁₂ alkylene, C₁₋₆ alkylene, C₁₋₅ alkylene, C₁₋₄ alkylene, C₁₋₃ alkylene, or C₁₋₂ alkylene. In some embodiments, Q is —(C₁₋₆ alkylene)C(O)—, —(C₁₋₃ alkylene)C(O)—, —(C₁₋₂ alkylene)C(O)—, or —CH₂—C(O)—. In some embodiments, Q is C₃₋₉ cycloalkylene, C₃₋₆ cycloalkylene, C₃₋₅ cycloalkylene, or C₃₋₄ cycloalkylene. In some embodiments, Q is —(C₃₋₉ cycloalkylene)C(O)—, —(C₃₋₆ cycloalkylene)C(O)—, —(C₃₋₅ cycloalkylene)C(O)—, or —(C₃₋₄ cycloalkylene)C(O)—. In some embodiments, Q is —C(O)—. In some embodiments, Q is —S(O)₂—.

In some embodiments of the compound of formula (I), (I-A), (I-B), (I-C), (I-D), (I-E), (I-F), (I-G), (I-A-1), (I-A-2), (I-A-3), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, Q is a absent.

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, Q is —CH₂—, —CH₂CH₂—, —CH(CH₃)—, or —C(CH₃)₂—. In some embodiments, Q is —CH₂—.

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, Q is —C(O)—.

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, Q is —S(O)₂—.

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1a), (I), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or III-G-1), or a pharmaceutically acceptable salt, stereoisomer, or deuterated form thereof, Q is

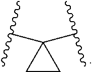 [SMILES: *C1(*)CC1]

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, Q is —CH₂C(O)—.

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, Q is

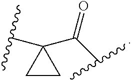 [SMILES: *C(=O)C1(*)CC1]

R³ and R⁴

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, R³ is carbocycle, heterocycle, aryl, heteroaryl, or —NR^{X}R^{Y}, wherein the carbocycle, heterocycle, aryl, and heteroaryl are each optionally substituted, R^{X} is H, C₁₋₆ alkyl, C₁₋₆ haloalkyl, or C₁₋₆ hydroxyalkyl, and R^{Y} is C₃₋₈ cycloalkyl, C₃₋₈ hydroxycycloalkyl, aryl, or heterocycle. In some embodiments, the carbocycle, heterocycle, aryl, and heteroaryl are each optionally substituted with 1, 2, or 3 R⁴, wherein each R⁴ is independently halogen, —OH, C₁₋₆ alkyl, C₁₋₆ alkoxy, or —C(O)O—(C₁₋₆ alkyl).

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, R³ is C₃₋₈ carbocycle, 3-10 membered heterocycle, C₆₋₁₀ aryl, or 5-10 membered heteroaryl, and wherein the C₃₋₈ carbocycle, 3-8 membered heterocycle, C₆₋₁₀ aryl, and 5-10 membered heteroaryl are each optionally substituted. In some embodiments, the C₃₋₈ carbocycle, 3-8 membered heterocycle, C₆₋₁₀ aryl, and 5-10 membered heteroaryl are each optionally substituted with 1 or 2 R⁴, and each R⁴ is independently halogen, —OH, C₁₋₆ alkyl, C₁₋₆ alkoxy, or —C(O)O—C₁₋₆ alkyl.

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, R³ is C₆₋₁₀ aryl optionally substituted with 1 or 2 R⁴, and wherein each R⁴ is independently halogen, C₁₋₆ alkyl, or C₁₋₆ alkoxy.

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, R³ is C₆₋₁₀ aryl optionally substituted with —F, —Cl, or C₁₋₃ alkoxy. In some embodiments, R³ is

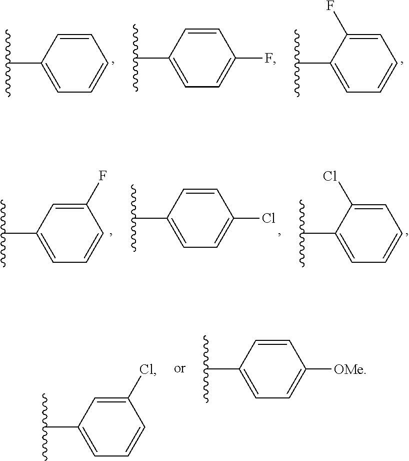

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, R³ is 5-10 membered heteroaryl optionally substituted with 1 or 2 R⁴; and wherein the heteroaryl contains 1 or 2 heteroatoms selected from N, S, or O. In some embodiments, R³ is

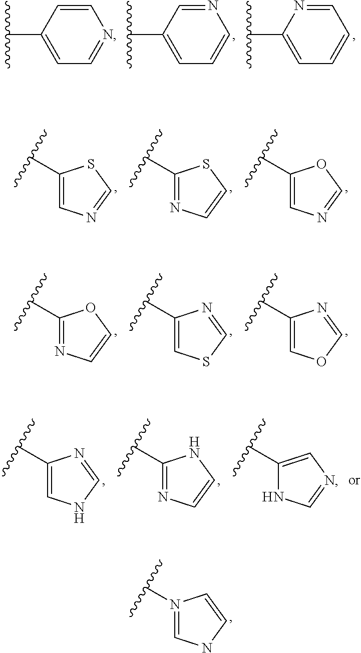

each optionally substituted with one R⁴. In some embodiments, R³ is

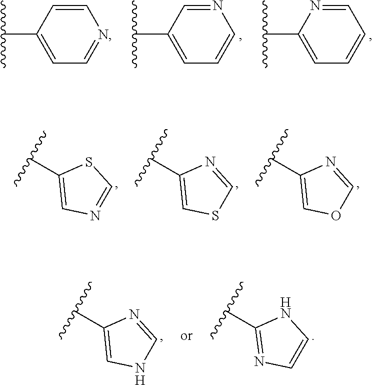

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, R³ is C₃₋₈ carbocycle optionally substituted with 1 or 2 R⁴, and wherein each R⁴ is independently halogen, —OH, C₁₋₆ alkyl, or C₁₋₆ alkoxy. In some embodiments, R³ is

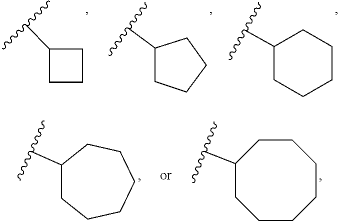

each optionally substituted with one or two R⁴. In some embodiments, R⁴ is halogen. In some embodiments, each R⁴ is fluoro. In some embodiments, R³ is

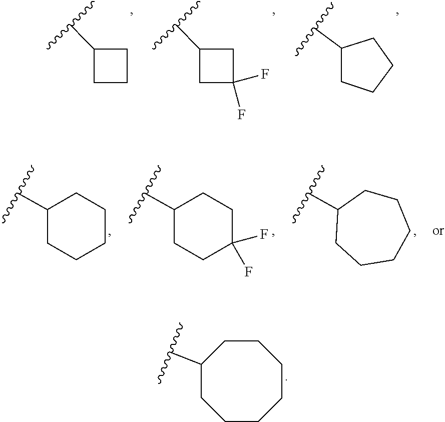

In some embodiments, R³ is

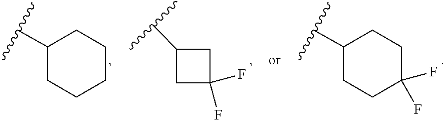 [SMILES: *C1CCCCC1 ; *C1CC(F)(F)C1 ; *C1CCC(F)(F)CC1]

In some embodiments, R³ is

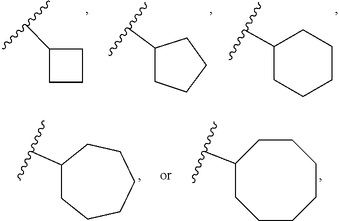

each optionally substituted with one R⁴. In some embodiments, R³ is

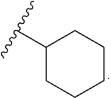 [SMILES: *C1CCCCC1]

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, R³ is 3-10 membered heterocycle containing 1 or 2 heteroatoms selected from N, O, or S, and the heterocycle is optionally substituted with 1 or 2 R⁴, and wherein each R⁴ is independently —OH, C₁₋₆ alkyl, or —C(O)O—(C₁₋₆ alkyl). In some embodiments, each R⁴ is independently —OH, C₁₋₃ alkyl, or —C(O)O—(C₁₋₅ alkyl). In some embodiments, R³ is

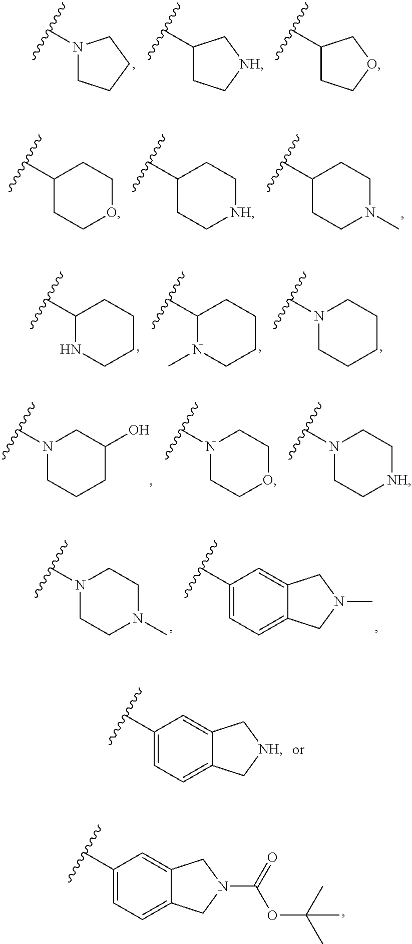

each optionally substituted with 1 or 2 R⁴. In some embodiments, R³ is

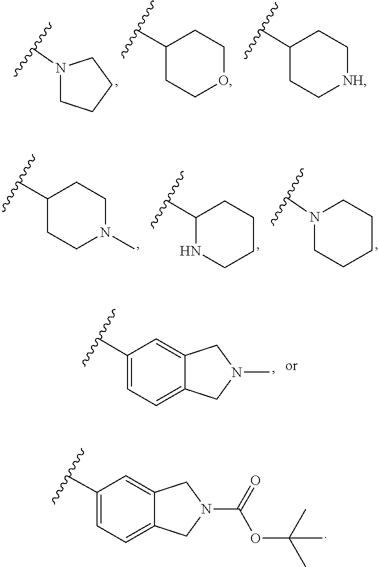

### -Q-R³

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, -Q-R³ is

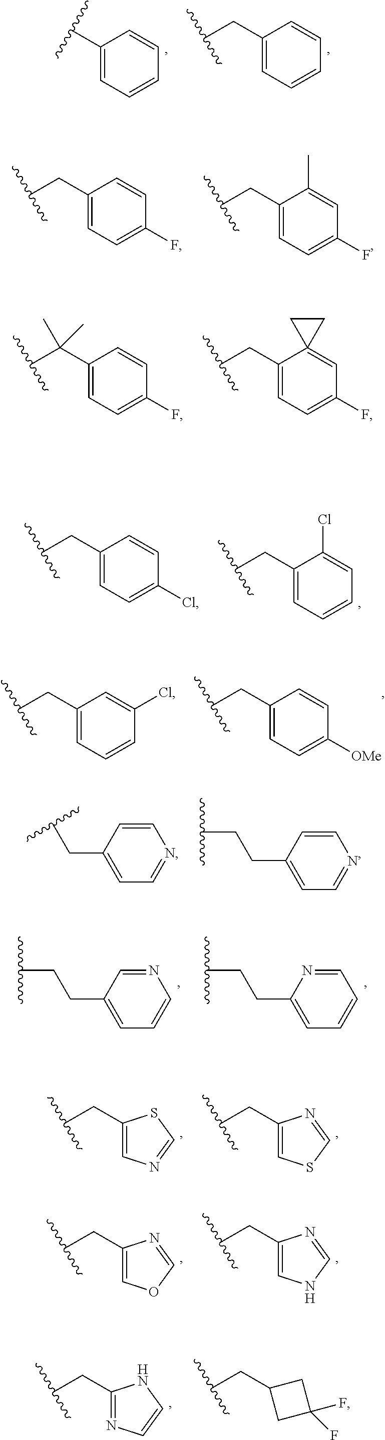

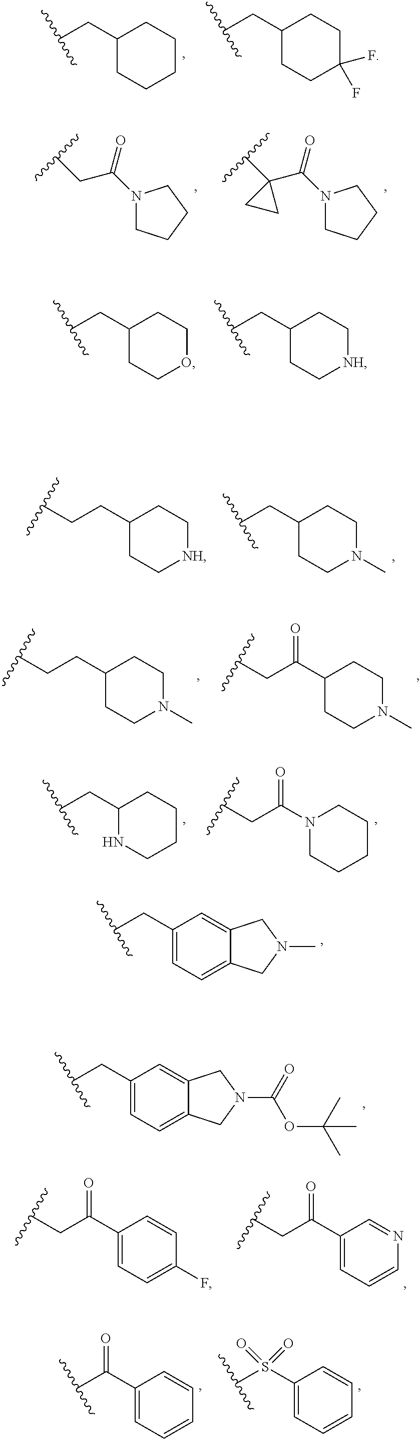

or —CH₂CH₂—NH—CH(CH₃)₂. In some embodiments, -Q-R³ is

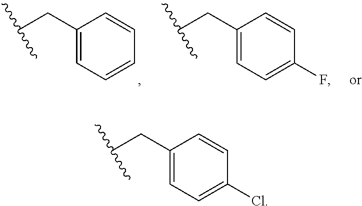

In some embodiments of the compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof, -Q-R³ is

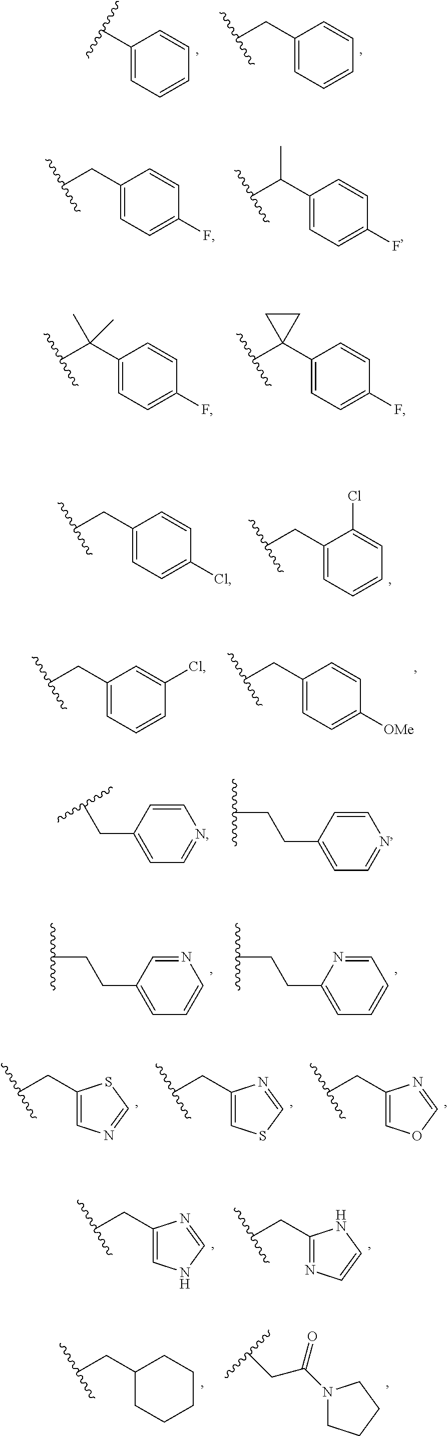

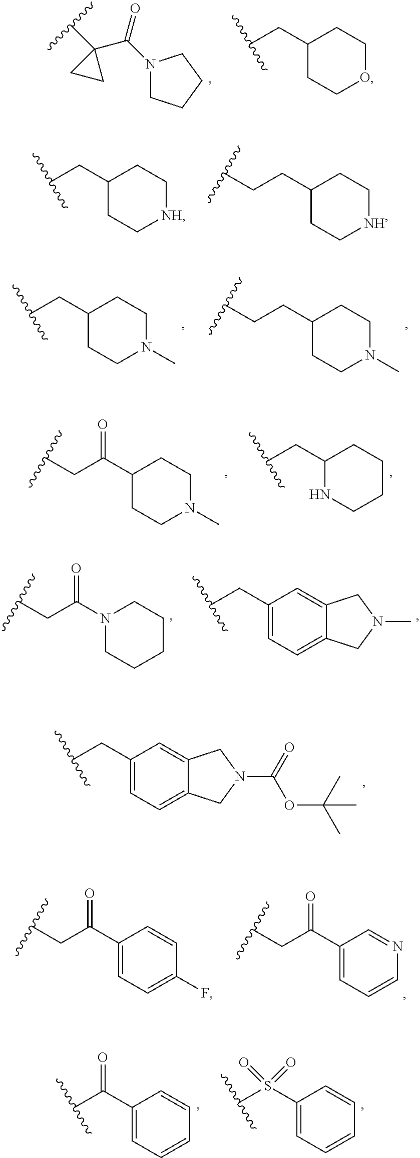

or —CH₂CH₂—NH—CH(CH₃)₂. In some embodiments, -Q-R³ is

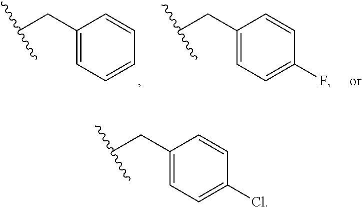

In embodiments, the compounds disclosed herein (e.g., compounds of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt, stereoisomer, or deuterated form thereof) have a molecular weight of up to 1,500 Daltons (Da), up to 1,200 Da, up to 1,000 Da, up to 900 Da, up to 800 Da, up to 700 Da, up to 600 Da, or up to 500 Da. In some embodiments, the compounds disclosed herein have a molecular weight ranging from about 100 Da to about 2,000 Da, from about 200 Da to about 1,800 Da, from about 400 Da to about 1,600 Da, from about 600 Da to about 1,200 Da, from about 700 Da to about 1,000 Da, or from about 800 Da to about 900 Da.

Another embodiment is a product obtainable by any of the processes or examples disclosed herein.

In embodiments, provided herein is a compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a stereoisomer thereof, or a pharmaceutically acceptable salt thereof.

In embodiments, provided herein is a stereoisomer of a compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1). In embodiments, provided herein is a pharmaceutically acceptable salt of a compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1). Further embodiments of the disclosure relate to a deuterated compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or a pharmaceutically acceptable salt thereof.

The compound disclosed herein is not

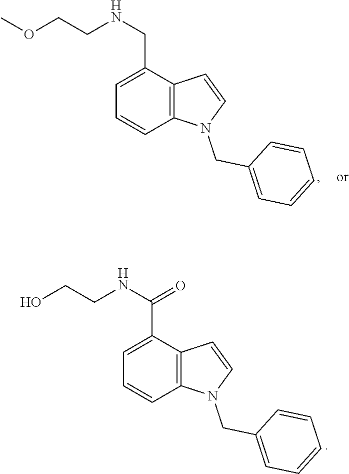 [SMILES: COCCNCc1cccc2c1ccn2Cc1ccccc1 ; O=C(NCCO)c1cccc2c1ccn2Cc1ccccc1]

In embodiments, provided herein is a compound in Table 1, 2, or 3, or a pharmaceutically acceptable salt thereof, racemic form thereof, or stereoisomer thereof.

In embodiments, provided herein is a compound in Table 1, 2, or 3, or a pharmaceutically acceptable salt thereof, or stereoisomer thereof.

In embodiments, provided herein is a compound in Table 1, 2, or 3, or a pharmaceutically acceptable salt thereof.

In one embodiment, provided herein is a compound set forth in Table 1, 2, or 3.

In some embodiments, provided herein is a pharmaceutically acceptable salt of a compound in Table 1, 2, or 3.

TABLE 1
Various compounds of the disclosure (series I)

| Comp. | |
|---|---|
| ID | Structure |
| 1 | 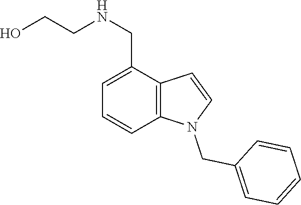 [SMILES: OCCNCc1cccc2c1ccn2Cc1ccccc1] |
| 2 | 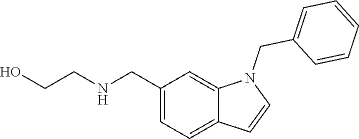 [SMILES: OCCNCc1ccc2ccn(Cc3ccccc3)c2c1] |
| 3 | 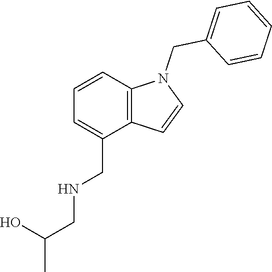 [SMILES: CC(O)CNCc1cccc2c1ccn2Cc1ccccc1] |
| 4 | 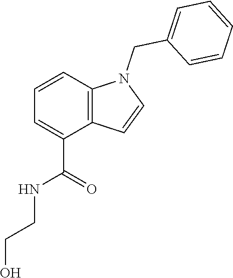 [SMILES: O=C(NCCO)c1cccc2c1ccn2Cc1ccccc1] |
| 5 | 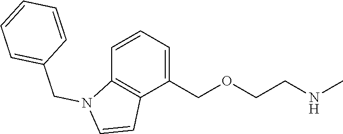 [SMILES: CNCCOCc1cccc2c1ccn2Cc1ccccc1] |
| 6 | 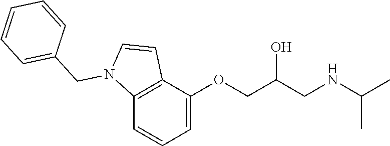 [SMILES: CC(C)NCC(O)COc1cccc2c1ccn2Cc1ccccc1] |
| 7 | 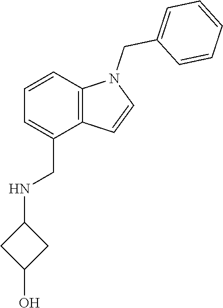 [SMILES: OC1CC(NCc2cccc3c2ccn3Cc2ccccc2)C1] |
| 8 | 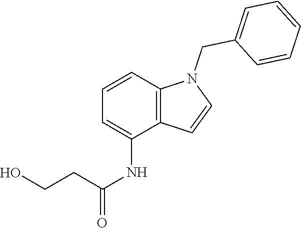 [SMILES: O=C(CCO)Nc1cccc2c1ccn2Cc1ccccc1] |
| 9 | 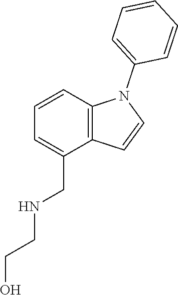 [SMILES: OCCNCc1cccc2c1ccn2-c1ccccc1] |
| 10 | 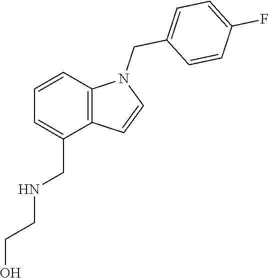 [SMILES: OCCNCc1cccc2c1ccn2Cc1ccc(F)cc1] |
| 11 | 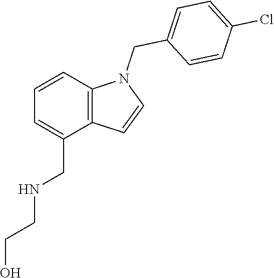 [SMILES: OCCNCc1cccc2c1ccn2Cc1ccc(Cl)cc1] |
| 12 | 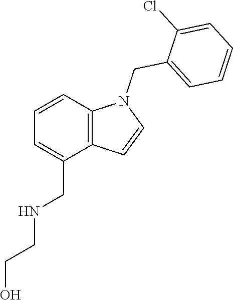 [SMILES: OCCNCc1cccc2c1ccn2Cc1ccccc1Cl] |
| 13 | 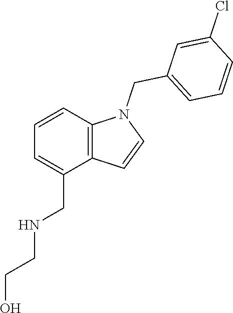 [SMILES: OCCNCc1cccc2c1ccn2Cc1cccc(Cl)c1] |
| 14 | 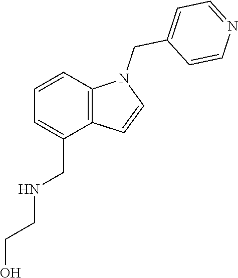 [SMILES: OCCNCc1cccc2c1ccn2Cc1ccncc1] |
| 15 | 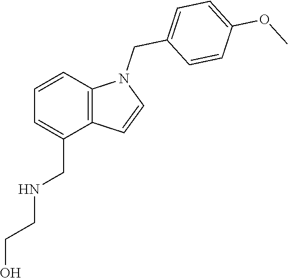 [SMILES: COc1ccc(Cn2ccc3c(CNCCO)cccc32)cc1] |
| 16 | 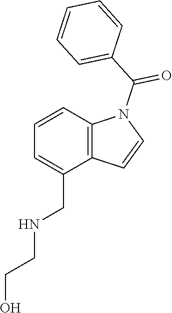 [SMILES: O=C(c1ccccc1)n1ccc2c(CNCCO)cccc21] |
| 17 | 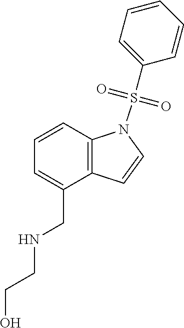 [SMILES: O=S(=O)(c1ccccc1)n1ccc2c(CNCCO)cccc21] |
| 18 | 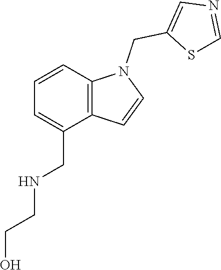 [SMILES: OCCNCc1cccc2c1ccn2Cc1cncs1] |
| 19 | 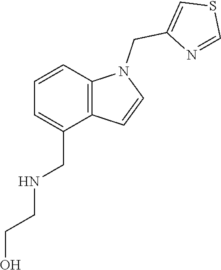 [SMILES: OCCNCc1cccc2c1ccn2Cc1cscn1] |
| 20 | 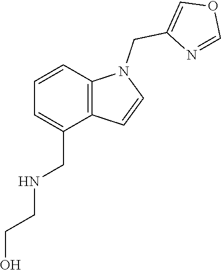 [SMILES: OCCNCc1cccc2c1ccn2Cc1cocn1] |
| 21 | 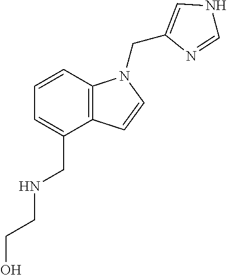 [SMILES: OCCNCc1cccc2c1ccn2Cc1c[nH]cn1] |
| 22 | 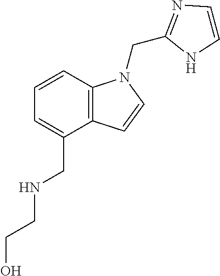 [SMILES: OCCNCc1cccc2c1ccn2Cc1ncc[nH]1] |
| 23 | 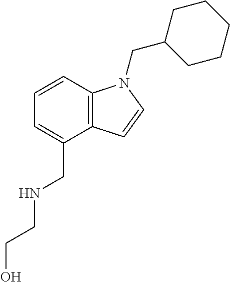 [SMILES: OCCNCc1cccc2c1ccn2CC1CCCCC1] |
| 24 | 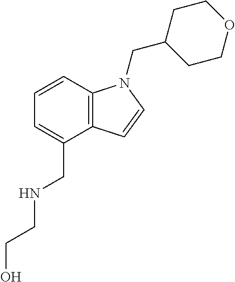 [SMILES: OCCNCc1cccc2c1ccn2CC1CCOCC1] |
| 25 | 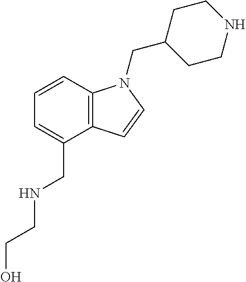 [SMILES: OCCNCc1cccc2c1ccn2CC1CCNCC1] |
| 26 | 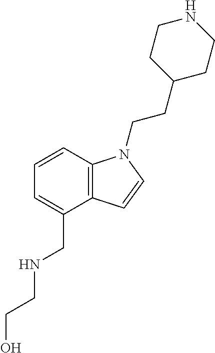 [SMILES: OCCNCc1cccc2c1ccn2CCC1CCNCC1] |
| 27 | 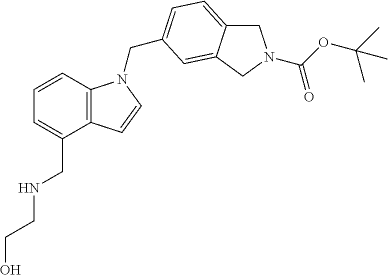 [SMILES: CC(C)(C)OC(=O)N1Cc2ccc(Cn3ccc4c(CNCCO)cccc43)cc2C1] |
| 28 | 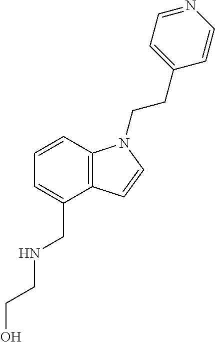 [SMILES: OCCNCc1cccc2c1ccn2CCc1ccncc1] |
| 29 | 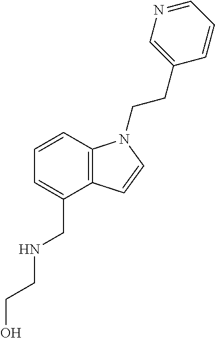 [SMILES: OCCNCc1cccc2c1ccn2CCc1cccnc1] |
| 30 | 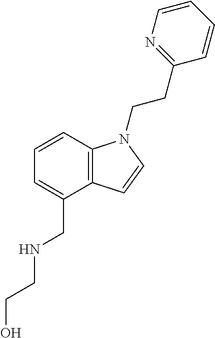 [SMILES: OCCNCc1cccc2c1ccn2CCc1ccccn1] |
| 31 | 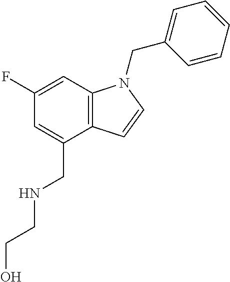 [SMILES: OCCNCc1cc(F)cc2c1ccn2Cc1ccccc1] |
| 32 | 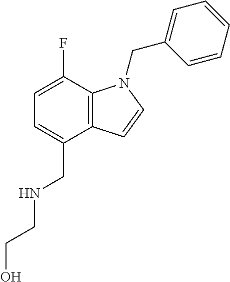 [SMILES: OCCNCc1ccc(F)c2c1ccn2Cc1ccccc1] |
| 33 | 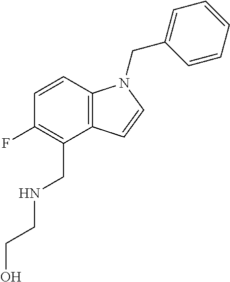 [SMILES: OCCNCc1c(F)ccc2c1ccn2Cc1ccccc1] |
| 34 | 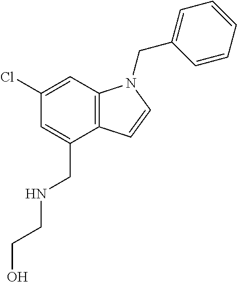 [SMILES: OCCNCc1cc(Cl)cc2c1ccn2Cc1ccccc1] |
| 35 | 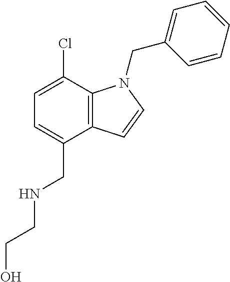 [SMILES: OCCNCc1ccc(Cl)c2c1ccn2Cc1ccccc1] |
| 36 | 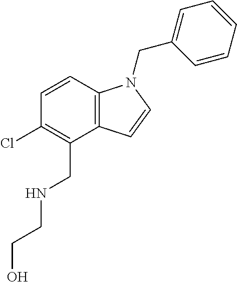 [SMILES: OCCNCc1c(Cl)ccc2c1ccn2Cc1ccccc1] |
| 37 | 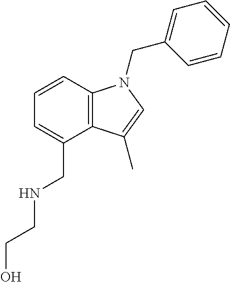 [SMILES: Cc1cn(Cc2ccccc2)c2cccc(CNCCO)c12] |
| 38 | 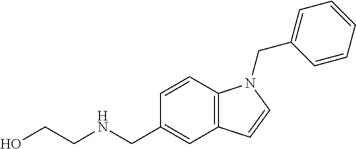 [SMILES: OCCNCc1ccc2c(ccn2Cc2ccccc2)c1] |
| 39 | 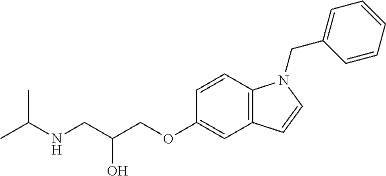 [SMILES: CC(C)NCC(O)COc1ccc2c(ccn2Cc2ccccc2)c1] |
| 40 | 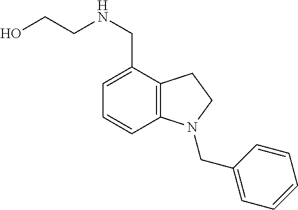 [SMILES: OCCNCc1cccc2c1CCN2Cc1ccccc1] |
| 41 | 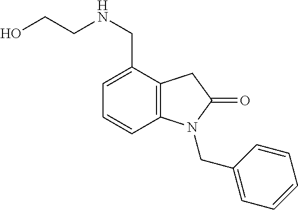 [SMILES: O=C1Cc2c(CNCCO)cccc2N1Cc1ccccc1] |
| 42 | 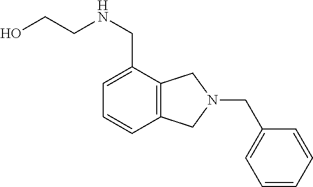 [SMILES: OCCNCc1cccc2c1CN(Cc1ccccc1)C2] |
| 43 | 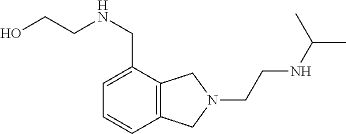 [SMILES: CC(C)NCCN1Cc2cccc(CNCCO)c2C1] |
| 44 | 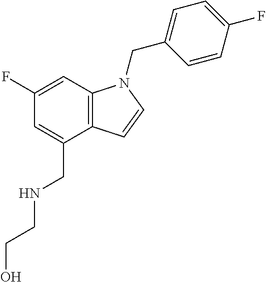 [SMILES: OCCNCc1cc(F)cc2c1ccn2Cc1ccc(F)cc1] |
| 45 | 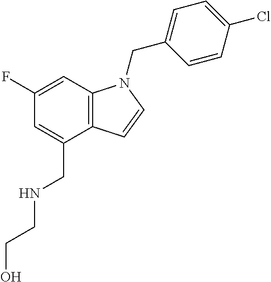 [SMILES: OCCNCc1cc(F)cc2c1ccn2Cc1ccc(Cl)cc1] |
| 46 | 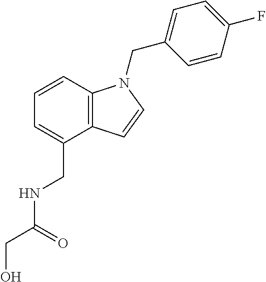 [SMILES: O=C(CO)NCc1cccc2c1ccn2Cc1ccc(F)cc1] |
| 47 | 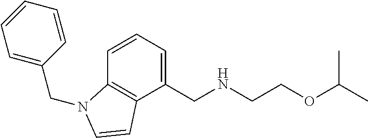 [SMILES: CC(C)OCCNCc1cccc2c1ccn2Cc1ccccc1] |
| 48 | 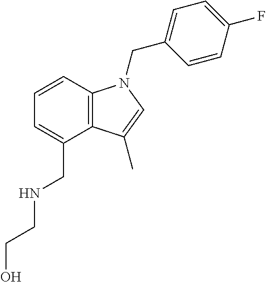 [SMILES: Cc1cn(Cc2ccc(F)cc2)c2cccc(CNCCO)c12] |
| 49 | 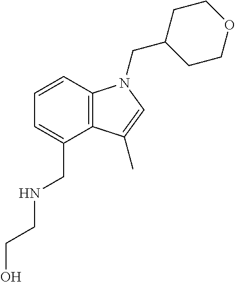 [SMILES: Cc1cn(CC2CCOCC2)c2cccc(CNCCO)c12] |
| 50 | 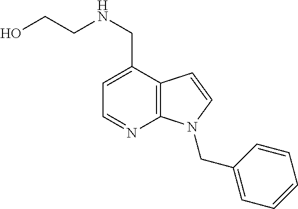 [SMILES: OCCNCc1ccnc2c1ccn2Cc1ccccc1] |
| 51 | 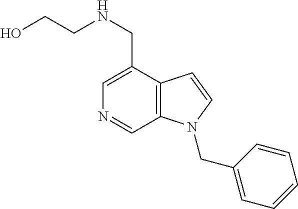 [SMILES: OCCNCc1cncc2c1ccn2Cc1ccccc1] |
| 52 | 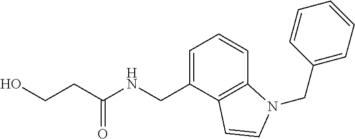 [SMILES: O=C(CCO)NCc1cccc2c1ccn2Cc1ccccc1] |
| 53 | 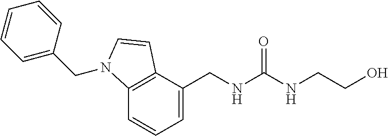 [SMILES: O=C(NCCO)NCc1cccc2c1ccn2Cc1ccccc1] |
| 54 | 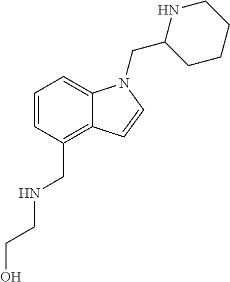 [SMILES: OCCNCc1cccc2c1ccn2CC1CCCCN1] |
| 55 | 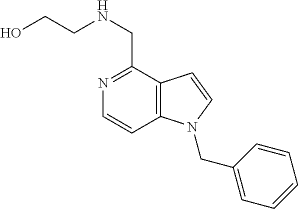 [SMILES: OCCNCc1nccc2c1ccn2Cc1ccccc1] |
| 56 | 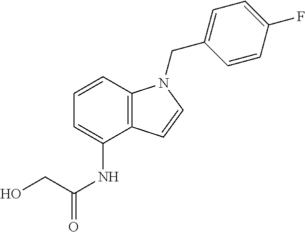 [SMILES: O=C(CO)Nc1cccc2c1ccn2Cc1ccc(F)cc1] |
| 57 | 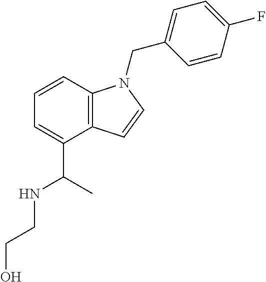 [SMILES: CC(NCCO)c1cccc2c1ccn2Cc1ccc(F)cc1] |
| 58 | 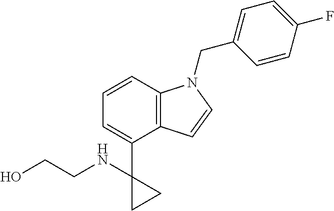 [SMILES: OCCNC1(c2cccc3c2ccn3Cc2ccc(F)cc2)CC1] |
| 59 | 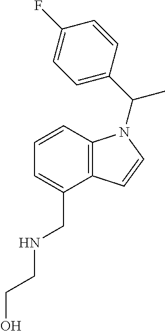 [SMILES: CC(c1ccc(F)cc1)n1ccc2c(CNCCO)cccc21] |
| 60 | 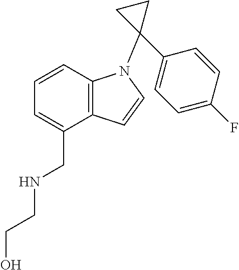 [SMILES: OCCNCc1cccc2c1ccn2C1(c2ccc(F)cc2)CC1] |
| 61 | 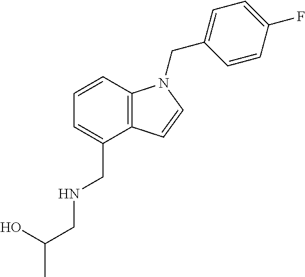 [SMILES: CC(O)CNCc1cccc2c1ccn2Cc1ccc(F)cc1] |
| 62 | 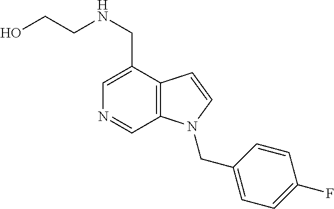 [SMILES: OCCNCc1cncc2c1ccn2Cc1ccc(F)cc1] |
| 63 | 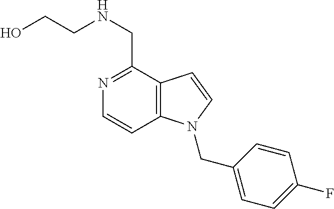 [SMILES: OCCNCc1nccc2c1ccn2Cc1ccc(F)cc1] |
| 64 | 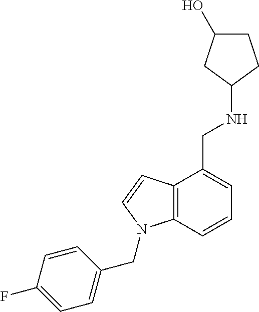 [SMILES: OC1CCC(NCc2cccc3c2ccn3Cc2ccc(F)cc2)C1] |
| 65 | 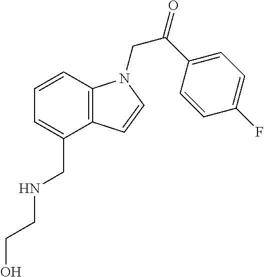 [SMILES: O=C(Cn1ccc2c(CNCCO)cccc21)c1ccc(F)cc1] |
| 66 | 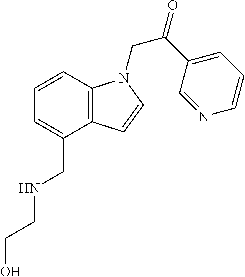 [SMILES: O=C(Cn1ccc2c(CNCCO)cccc21)c1cccnc1] |
| 67 | 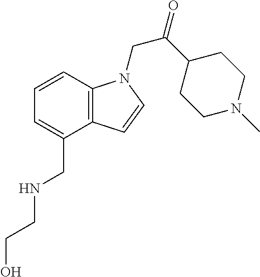 [SMILES: CN1CCC(C(=O)Cn2ccc3c(CNCCO)cccc32)CC1] |
| 68 | 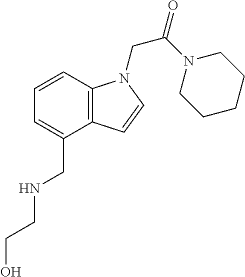 [SMILES: O=C(Cn1ccc2c(CNCCO)cccc21)N1CCCCC1] |
| 69 | 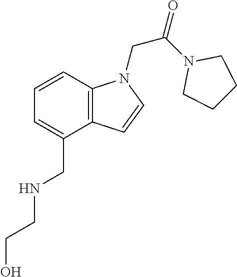 [SMILES: O=C(Cn1ccc2c(CNCCO)cccc21)N1CCCC1] |
| 70 | 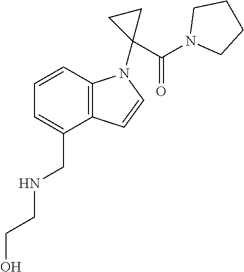 [SMILES: O=C(N1CCCC1)C1(n2ccc3c(CNCCO)cccc32)CC1] |
| 71 | 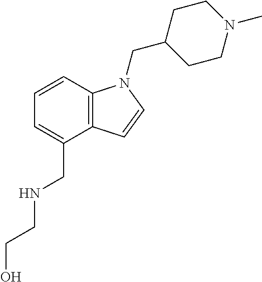 [SMILES: CN1CCC(Cn2ccc3c(CNCCO)cccc32)CC1] |
| 72 | 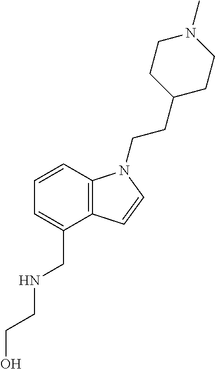 [SMILES: CN1CCC(CCn2ccc3c(CNCCO)cccc32)CC1] |
| 73 | 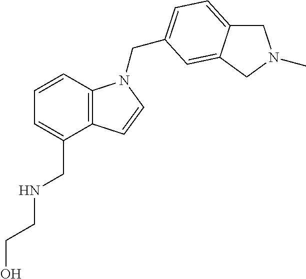 [SMILES: CN1Cc2ccc(Cn3ccc4c(CNCCO)cccc43)cc2C1] |
| 74 | 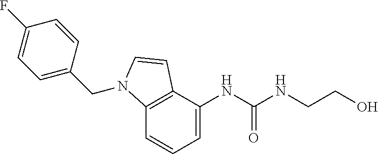 [SMILES: O=C(NCCO)Nc1cccc2c1ccn2Cc1ccc(F)cc1] |
| 75 | 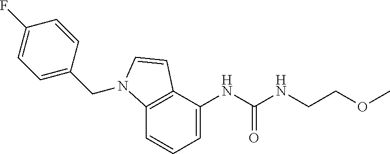 [SMILES: COCCNC(=O)Nc1cccc2c1ccn2Cc1ccc(F)cc1] |
| 76 | 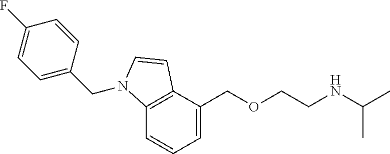 [SMILES: CC(C)NCCOCc1cccc2c1ccn2Cc1ccc(F)cc1] |
| 77 | 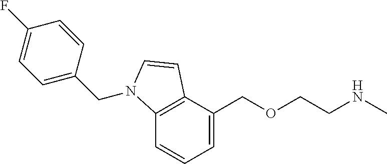 [SMILES: CNCCOCc1cccc2c1ccn2Cc1ccc(F)cc1] |
| 78 | 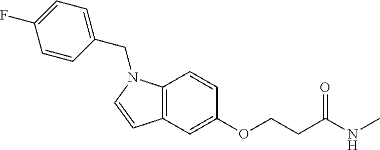 [SMILES: CNC(=O)CCOc1ccc2c(ccn2Cc2ccc(F)cc2)c1] |
| 79 | 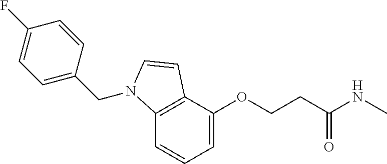 [SMILES: CNC(=O)CCOc1cccc2c1ccn2Cc1ccc(F)cc1] |
| 80 | 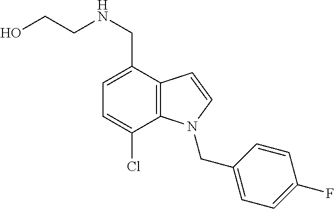 [SMILES: OCCNCc1ccc(Cl)c2c1ccn2Cc1ccc(F)cc1] |
| 81 | 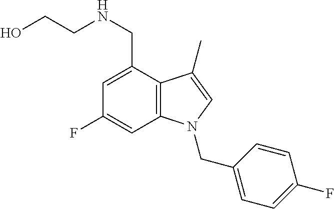 [SMILES: Cc1cn(Cc2ccc(F)cc2)c2cc(F)cc(CNCCO)c12] |
| 82 | 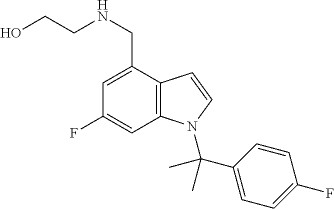 [SMILES: CC(C)(c1ccc(F)cc1)n1ccc2c(CNCCO)cc(F)cc21] |
| 83 | 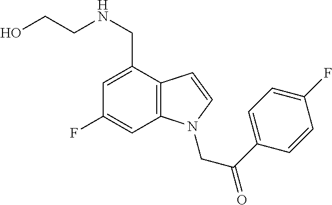 [SMILES: O=C(Cn1ccc2c(CNCCO)cc(F)cc21)c1ccc(F)cc1] |
| 108 | 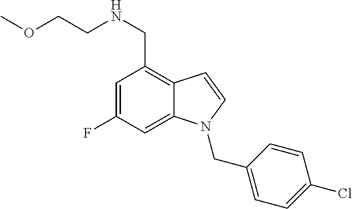 [SMILES: COCCNCc1cc(F)cc2c1ccn2Cc1ccc(Cl)cc1] |
| 110 | 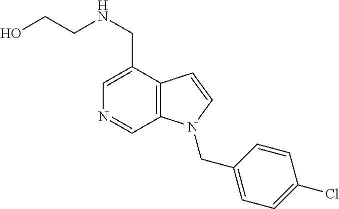 [SMILES: OCCNCc1cncc2c1ccn2Cc1ccc(Cl)cc1] |
| 111 | 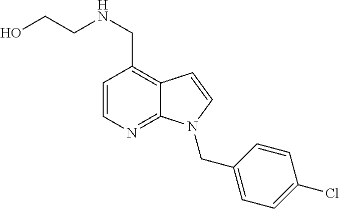 [SMILES: OCCNCc1ccnc2c1ccn2Cc1ccc(Cl)cc1] |
| 112 | 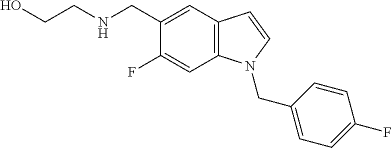 [SMILES: OCCNCc1cc2ccn(Cc3ccc(F)cc3)c2cc1F] |
| 113 | 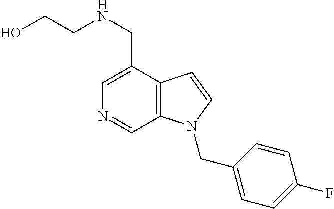 [SMILES: OCCNCc1cncc2c1ccn2Cc1ccc(F)cc1] |
| 114 | 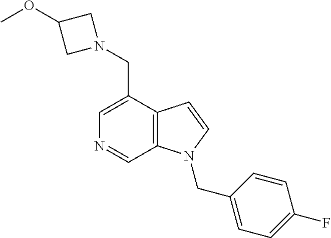 [SMILES: COC1CN(Cc2cncc3c2ccn3Cc2ccc(F)cc2)C1] |
| 115 | 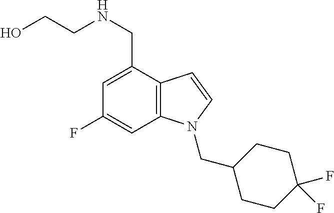 [SMILES: OCCNCc1cc(F)cc2c1ccn2CC1CCC(F)(F)CC1] |
| 116 | 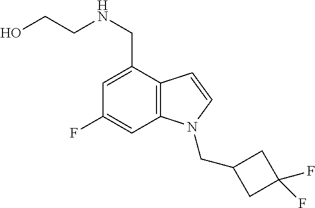 [SMILES: OCCNCc1cc(F)cc2c1ccn2CC1CC(F)(F)C1] |
| 178 | 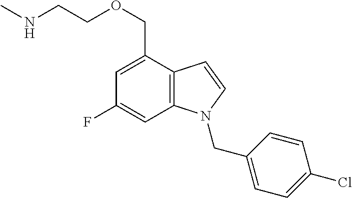 [SMILES: CNCCOCc1cc(F)cc2c1ccn2Cc1ccc(Cl)cc1] |
| 179 | 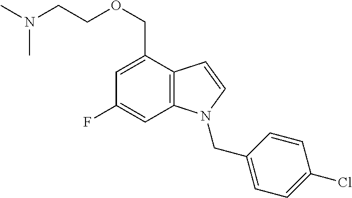 [SMILES: CN(C)CCOCc1cc(F)cc2c1ccn2Cc1ccc(Cl)cc1] |
| 180 | 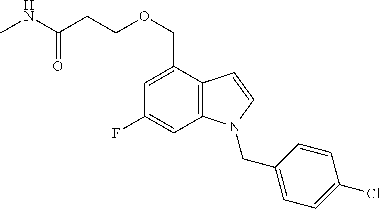 [SMILES: CNC(=O)CCOCc1cc(F)cc2c1ccn2Cc1ccc(Cl)cc1] |
| 181 | 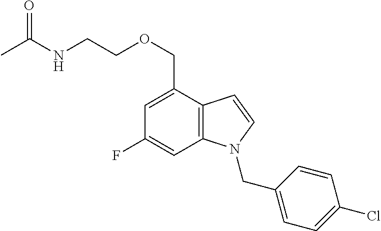 [SMILES: CC(=O)NCCOCc1cc(F)cc2c1ccn2Cc1ccc(Cl)cc1] |

TABLE 2
Various compounds of the disclosure (series II)

| Comp. | |
|---|---|
| ID | Structure |
| 84 | 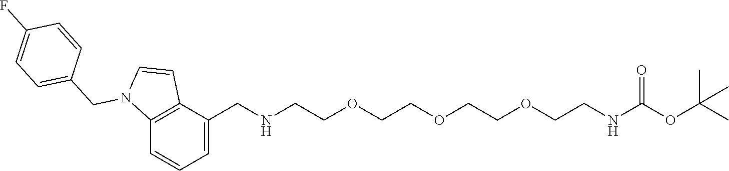 [SMILES: CC(C)(C)OC(=O)NCCOCCOCCOCCNCc1cccc2c1ccn2Cc1ccc(F)cc1] |
| 85 | 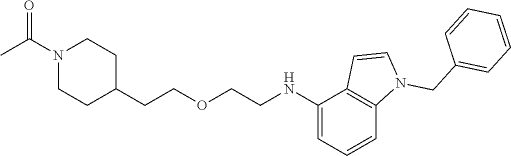 [SMILES: CC(=O)N1CCC(CCOCCNc2cccc3c2ccn3Cc2ccccc2)CC1] |
| 86 | 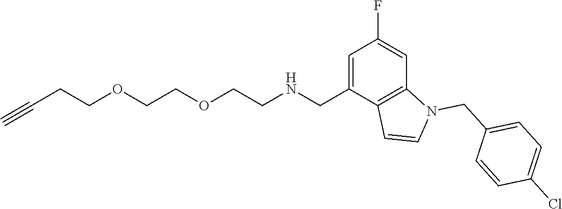 [SMILES: C#CCCOCCOCCNCc1cc(F)cc2c1ccn2Cc1ccc(Cl)cc1] |
| 87 | 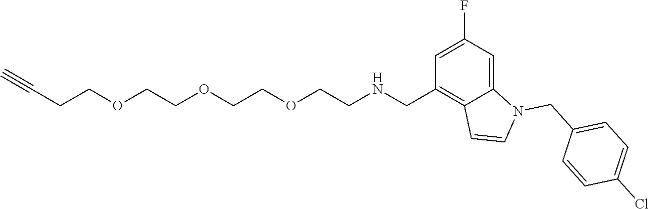 [SMILES: C#CCCOCCOCCOCCNCc1cc(F)cc2c1ccn2Cc1ccc(Cl)cc1] |
| 88 | 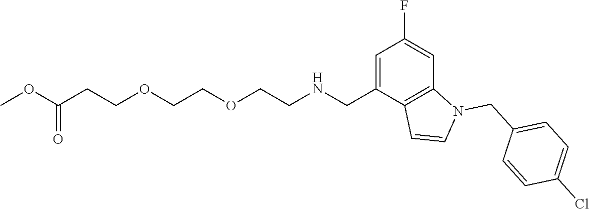 [SMILES: COC(=O)CCOCCOCCNCc1cc(F)cc2c1ccn2Cc1ccc(Cl)cc1] |
| 89 | 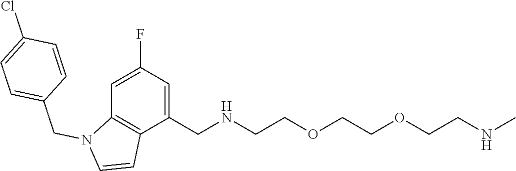 [SMILES: CNCCOCCOCCNCc1cc(F)cc2c1ccn2Cc1ccc(Cl)cc1] |
| 90 | 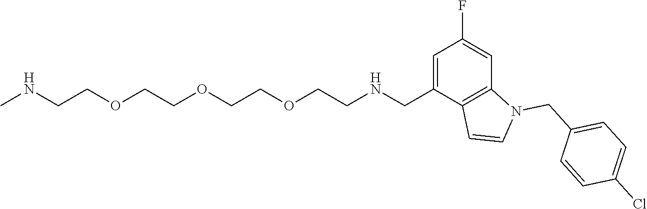 [SMILES: CNCCOCCOCCOCCNCc1cc(F)cc2c1ccn2Cc1ccc(Cl)cc1] |
| 91 | 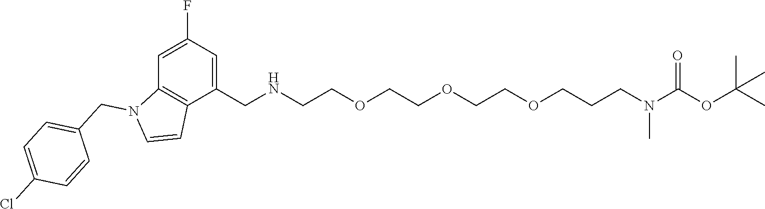 [SMILES: CN(CCCOCCOCCOCCNCc1cc(F)cc2c1ccn2Cc1ccc(Cl)cc1)C(=O)OC(C)(C)C] |
| 92 | 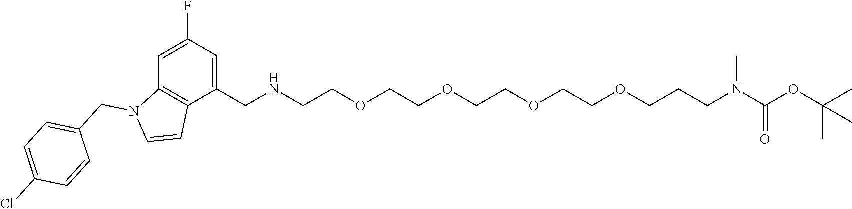 [SMILES: CN(CCCOCCOCCOCCOCCNCc1cc(F)cc2c1ccn2Cc1ccc(Cl)cc1)C(=O)OC(C)(C)C] |
| 93 | 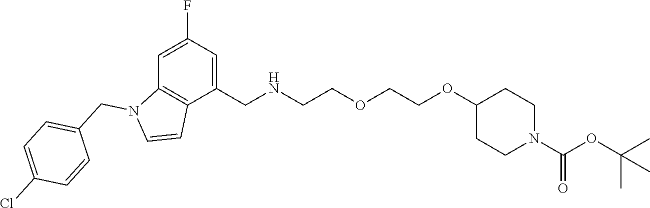 [SMILES: CC(C)(C)OC(=O)N1CCC(OCCOCCNCc2cc(F)cc3c2ccn3Cc2ccc(Cl)cc2)CC1] |
| 94 | 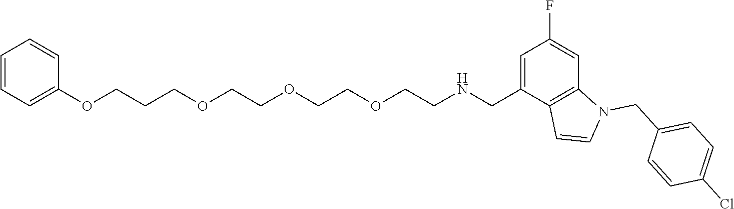 [SMILES: Fc1cc(CNCCOCCOCCOCCCOc2ccccc2)c2ccn(Cc3ccc(Cl)cc3)c2c1] |
| 95 | 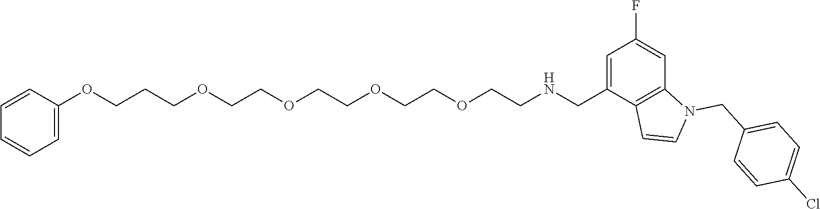 [SMILES: Fc1cc(CNCCOCCOCCOCCOCCCOc2ccccc2)c2ccn(Cc3ccc(Cl)cc3)c2c1] |
| 96 | 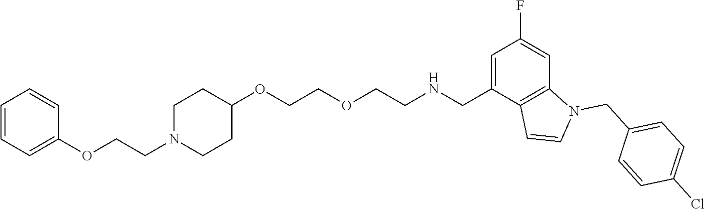 [SMILES: Fc1cc(CNCCOCCOC2CCN(CCOc3ccccc3)CC2)c2ccn(Cc3ccc(Cl)cc3)c2c1] |
| 117 | 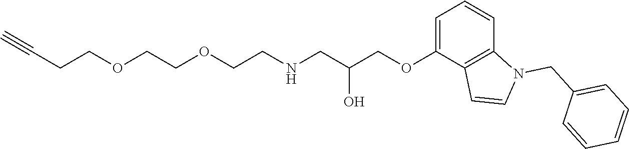 [SMILES: C#CCCOCCOCCNCC(O)COc1cccc2c1ccn2Cc1ccccc1] |
| 118 | 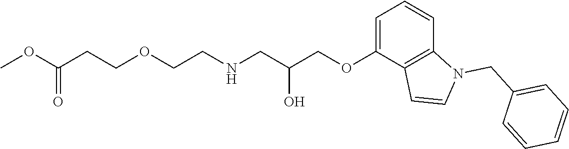 [SMILES: COC(=O)CCOCCNCC(O)COc1cccc2c1ccn2Cc1ccccc1] |
| 119 | 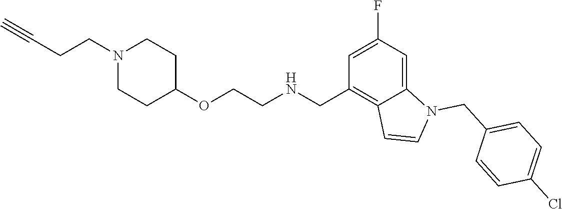 [SMILES: C#CCCN1CCC(OCCNCc2cc(F)cc3c2ccn3Cc2ccc(Cl)cc2)CC1] |
| 120 | 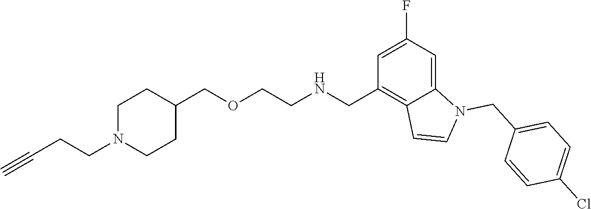 [SMILES: C#CCCN1CCC(COCCNCc2cc(F)cc3c2ccn3Cc2ccc(Cl)cc2)CC1] |
| 121 | 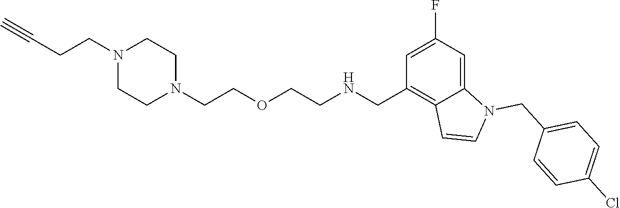 [SMILES: C#CCCN1CCN(CCOCCNCc2cc(F)cc3c2ccn3Cc2ccc(Cl)cc2)CC1] |
| 122 | 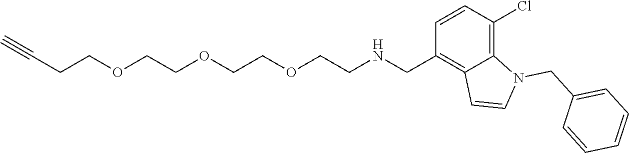 [SMILES: C#CCCOCCOCCOCCNCc1ccc(Cl)c2c1ccn2Cc1ccccc1] |
| 123 | 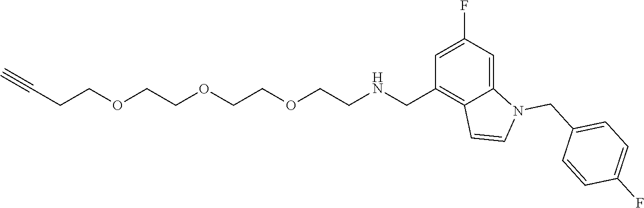 [SMILES: C#CCCOCCOCCOCCNCc1cc(F)cc2c1ccn2Cc1ccc(F)cc1] |
| 124 | 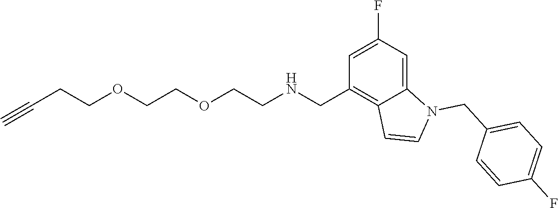 [SMILES: C#CCCOCCOCCNCc1cc(F)cc2c1ccn2Cc1ccc(F)cc1] |
| 125 | 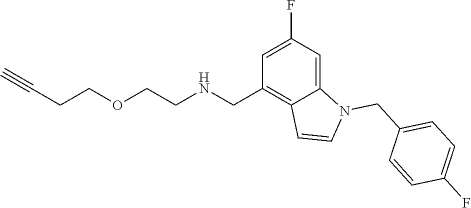 [SMILES: C#CCCOCCNCc1cc(F)cc2c1ccn2Cc1ccc(F)cc1] |
| 126 | 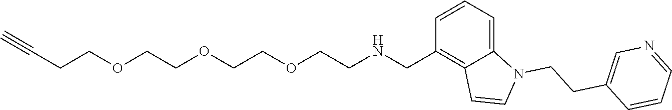 [SMILES: C#CCCOCCOCCOCCNCc1cccc2c1ccn2CCc1cccnc1] |
| 127 | 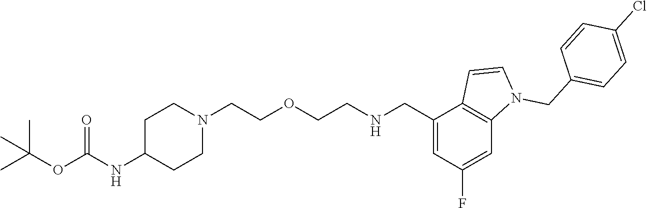 [SMILES: CC(C)(C)OC(=O)NC1CCN(CCOCCNCc2cc(F)cc3c2ccn3Cc2ccc(Cl)cc2)CC1] |
| 128 | 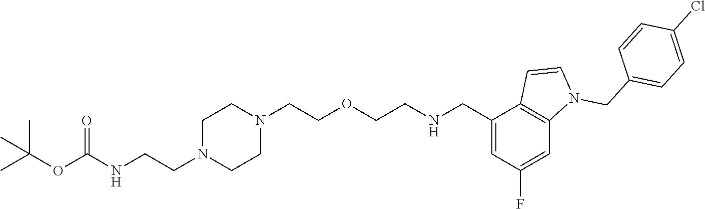 [SMILES: CC(C)(C)OC(=O)NCCN1CCN(CCOCCNCc2cc(F)cc3c2ccn3Cc2ccc(Cl)cc2)CC1] |
| 129 | 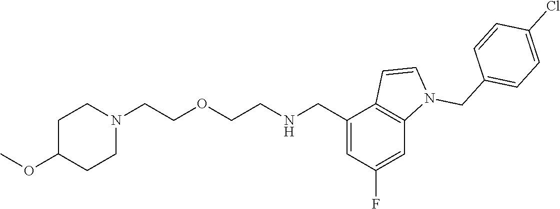 [SMILES: COC1CCN(CCOCCNCc2cc(F)cc3c2ccn3Cc2ccc(Cl)cc2)CC1] |
| 130 | 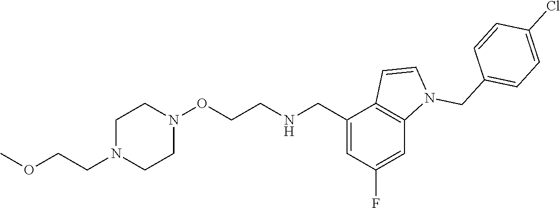 [SMILES: COCCN1CCN(OCCNCc2cc(F)cc3c2ccn3Cc2ccc(Cl)cc2)CC1] |
| 131 | 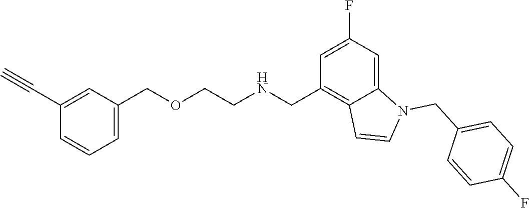 [SMILES: C#Cc1cccc(COCCNCc2cc(F)cc3c2ccn3Cc2ccc(F)cc2)c1] |
| 132 | 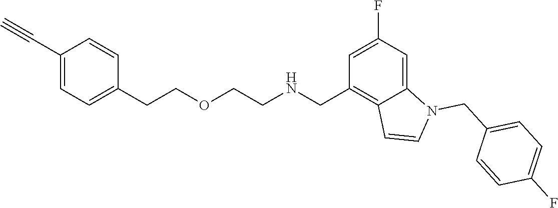 [SMILES: C#Cc1ccc(CCOCCNCc2cc(F)cc3c2ccn3Cc2ccc(F)cc2)cc1] |
| 133 | 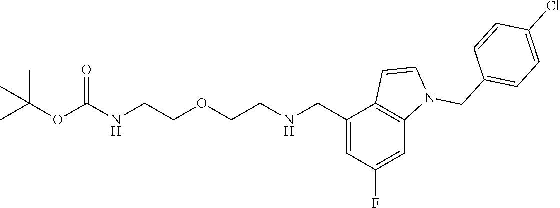 [SMILES: CC(C)(C)OC(=O)NCCOCCNCc1cc(F)cc2c1ccn2Cc1ccc(Cl)cc1] |
| 134 | 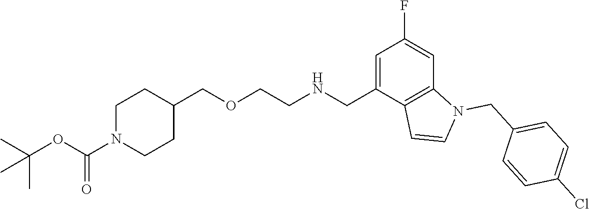 [SMILES: CC(C)(C)OC(=O)N1CCC(COCCNCc2cc(F)cc3c2ccn3Cc2ccc(Cl)cc2)CC1] |
| 135 | 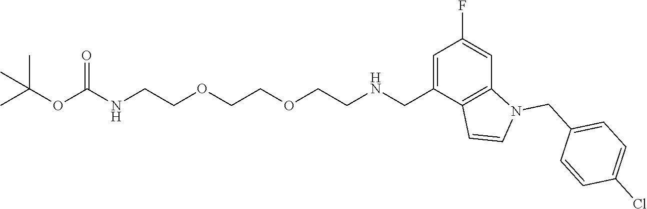 [SMILES: CC(C)(C)OC(=O)NCCOCCOCCNCc1cc(F)cc2c1ccn2Cc1ccc(Cl)cc1] |
| 136 | 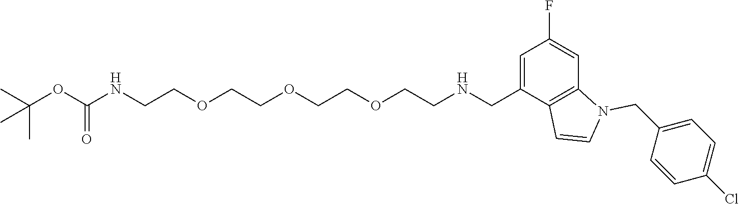 [SMILES: CC(C)(C)OC(=O)NCCOCCOCCOCCNCc1cc(F)cc2c1ccn2Cc1ccc(Cl)cc1] |
| 137 | 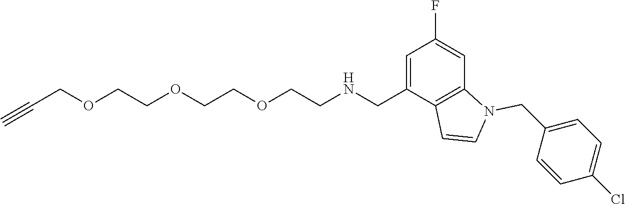 [SMILES: C#CCOCCOCCOCCNCc1cc(F)cc2c1ccn2Cc1ccc(Cl)cc1] |
| 138 | 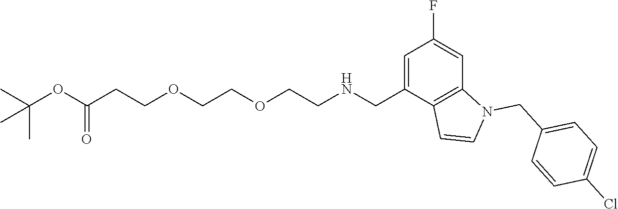 [SMILES: CC(C)(C)OC(=O)CCOCCOCCNCc1cc(F)cc2c1ccn2Cc1ccc(Cl)cc1] |
| 139 | 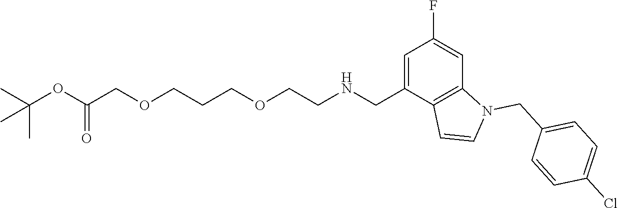 [SMILES: CC(C)(C)OC(=O)COCCCOCCNCc1cc(F)cc2c1ccn2Cc1ccc(Cl)cc1] |
| 140 | 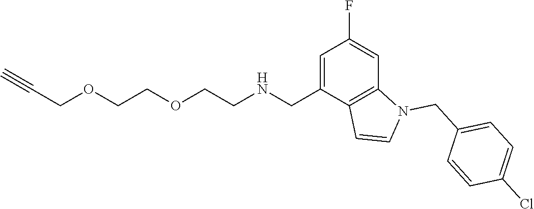 [SMILES: C#CCOCCOCCNCc1cc(F)cc2c1ccn2Cc1ccc(Cl)cc1] |
| 141 | 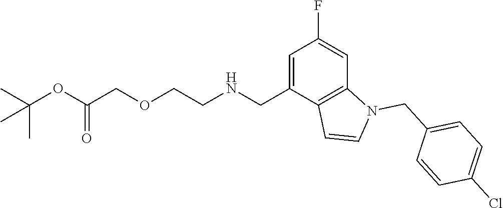 [SMILES: CC(C)(C)OC(=O)COCCNCc1cc(F)cc2c1ccn2Cc1ccc(Cl)cc1] |
| 142 | 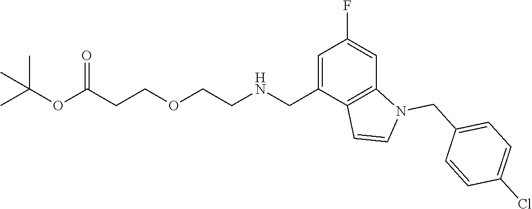 [SMILES: CC(C)(C)OC(=O)CCOCCNCc1cc(F)cc2c1ccn2Cc1ccc(Cl)cc1] |
| 143 | 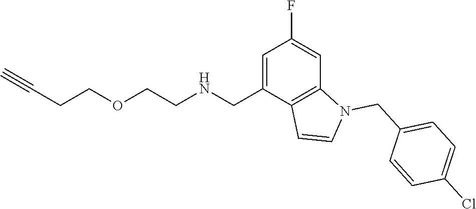 [SMILES: C#CCCOCCNCc1cc(F)cc2c1ccn2Cc1ccc(Cl)cc1] |
| 144 | 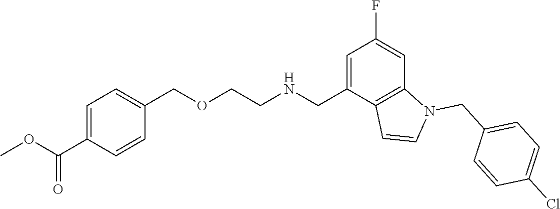 [SMILES: COC(=O)c1ccc(COCCNCc2cc(F)cc3c2ccn3Cc2ccc(Cl)cc2)cc1] |
| 145 | 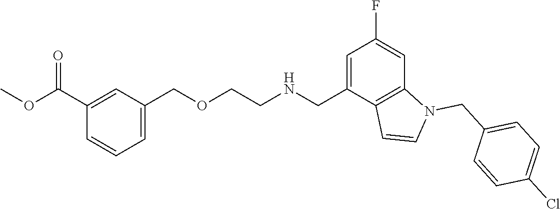 [SMILES: COC(=O)c1cccc(COCCNCc2cc(F)cc3c2ccn3Cc2ccc(Cl)cc2)c1] |
| 146 | 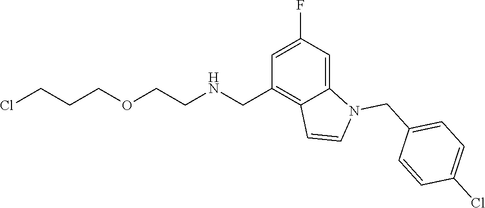 [SMILES: Fc1cc(CNCCOCCCCl)c2ccn(Cc3ccc(Cl)cc3)c2c1] |
| 147 | 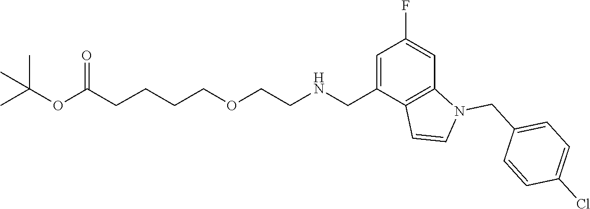 [SMILES: CC(C)(C)OC(=O)CCCCOCCNCc1cc(F)cc2c1ccn2Cc1ccc(Cl)cc1] |
| 148 | 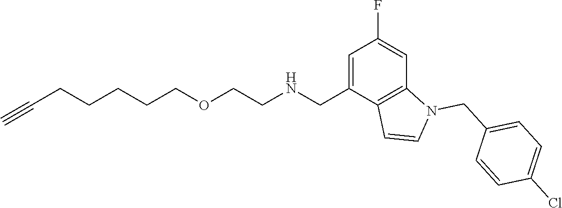 [SMILES: C#CCCCCCOCCNCc1cc(F)cc2c1ccn2Cc1ccc(Cl)cc1] |
| 149 | 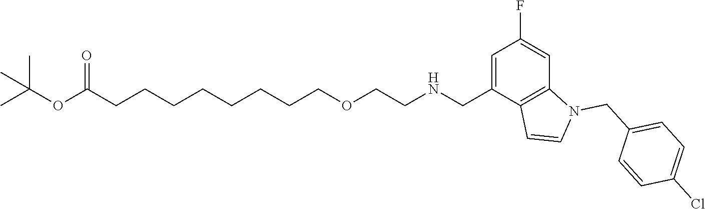 [SMILES: CC(C)(C)OC(=O)CCCCCCCCOCCNCc1cc(F)cc2c1ccn2Cc1ccc(Cl)cc1] |
| 150 | 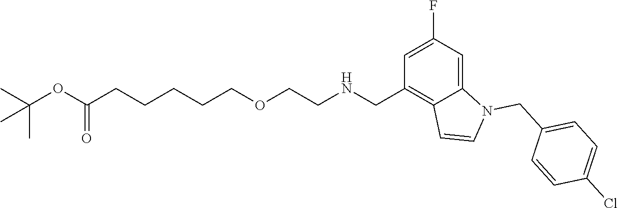 [SMILES: CC(C)(C)OC(=O)CCCCCOCCNCc1cc(F)cc2c1ccn2Cc1ccc(Cl)cc1] |
| 151 | 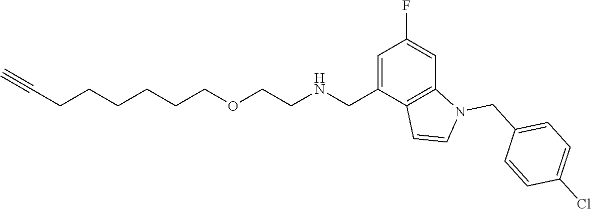 [SMILES: C#CCCCCCCOCCNCc1cc(F)cc2c1ccn2Cc1ccc(Cl)cc1] |
| 152 | 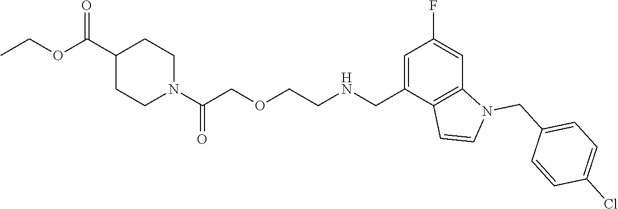 [SMILES: CCOC(=O)C1CCN(C(=O)COCCNCc2cc(F)cc3c2ccn3Cc2ccc(Cl)cc2)CC1] |
| 153 | 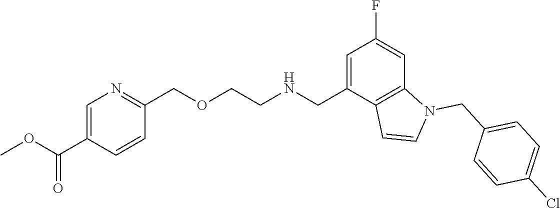 [SMILES: COC(=O)c1ccc(COCCNCc2cc(F)cc3c2ccn3Cc2ccc(Cl)cc2)nc1] |
| 154 | 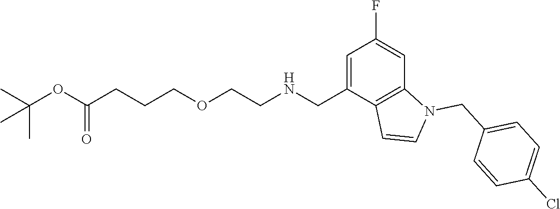 [SMILES: CC(C)(C)OC(=O)CCCOCCNCc1cc(F)cc2c1ccn2Cc1ccc(Cl)cc1] |
| 155 | 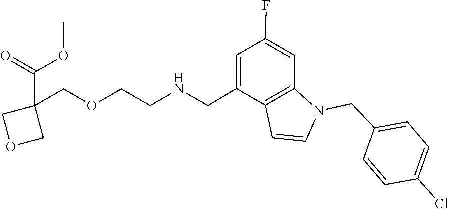 [SMILES: COC(=O)C1(COCCNCc2cc(F)cc3c2ccn3Cc2ccc(Cl)cc2)COC1] |
| 156 | 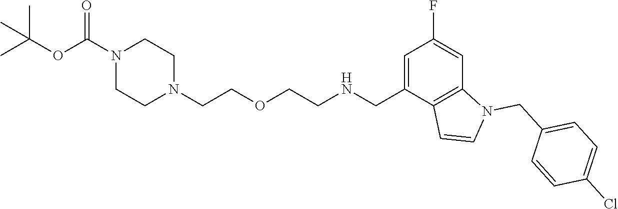 [SMILES: CC(C)(C)OC(=O)N1CCN(CCOCCNCc2cc(F)cc3c2ccn3Cc2ccc(Cl)cc2)CC1] |
| 157 | 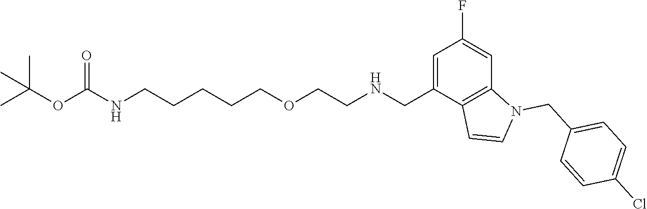 [SMILES: CC(C)(C)OC(=O)NCCCCCOCCNCc1cc(F)cc2c1ccn2Cc1ccc(Cl)cc1] |
| 158 | 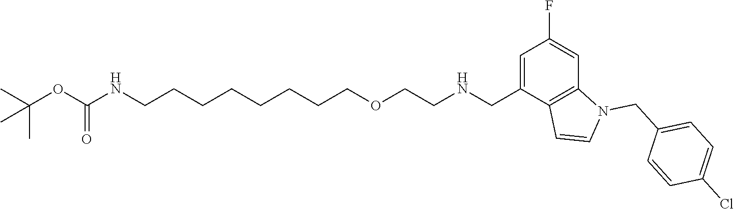 [SMILES: CC(C)(C)OC(=O)NCCCCCCCCOCCNCc1cc(F)cc2c1ccn2Cc1ccc(Cl)cc1] |
| 159 | 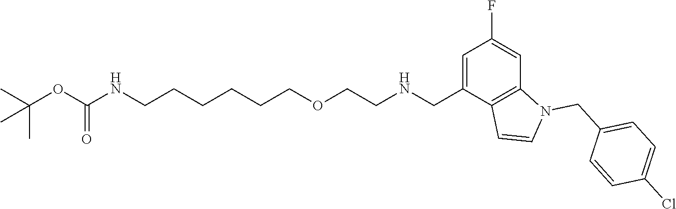 [SMILES: CC(C)(C)OC(=O)NCCCCCCOCCNCc1cc(F)cc2c1ccn2Cc1ccc(Cl)cc1] |
| 160 | 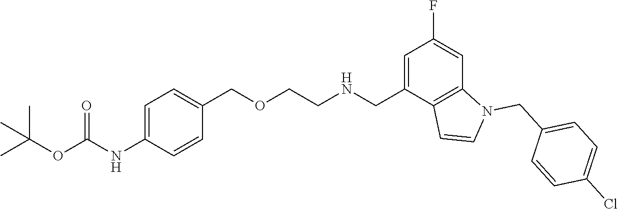 [SMILES: CC(C)(C)OC(=O)Nc1ccc(COCCNCc2cc(F)cc3c2ccn3Cc2ccc(Cl)cc2)cc1] |
| 161 | 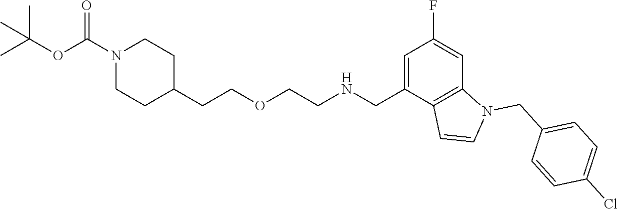 [SMILES: CC(C)(C)OC(=O)N1CCC(CCOCCNCc2cc(F)cc3c2ccn3Cc2ccc(Cl)cc2)CC1] |
| 162 | 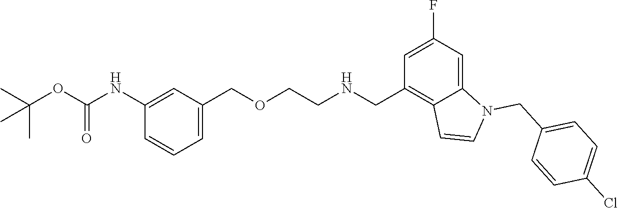 [SMILES: CC(C)(C)OC(=O)Nc1cccc(COCCNCc2cc(F)cc3c2ccn3Cc2ccc(Cl)cc2)c1] |
| 163 | 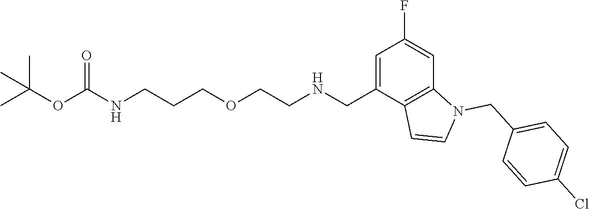 [SMILES: CC(C)(C)OC(=O)NCCCOCCNCc1cc(F)cc2c1ccn2Cc1ccc(Cl)cc1] |
| 164 | 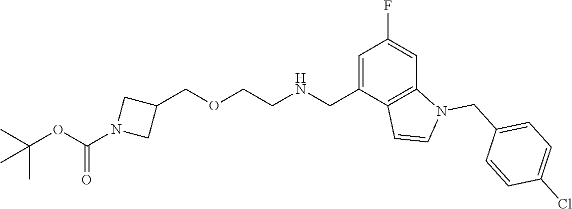 [SMILES: CC(C)(C)OC(=O)N1CC(COCCNCc2cc(F)cc3c2ccn3Cc2ccc(Cl)cc2)C1] |
| 165 | 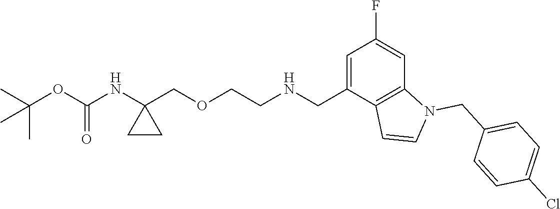 [SMILES: CC(C)(C)OC(=O)NC1(COCCNCc2cc(F)cc3c2ccn3Cc2ccc(Cl)cc2)CC1] |
| 166 | 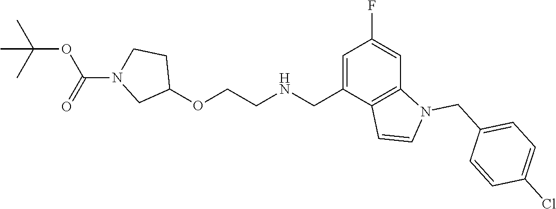 [SMILES: CC(C)(C)OC(=O)N1CCC(OCCNCc2cc(F)cc3c2ccn3Cc2ccc(Cl)cc2)C1] |
| 167 | 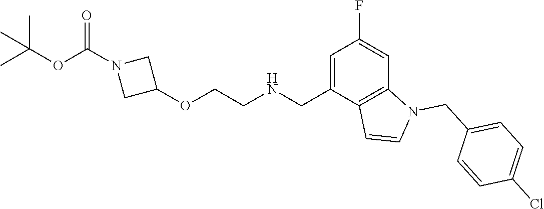 [SMILES: CC(C)(C)OC(=O)N1CC(OCCNCc2cc(F)cc3c2ccn3Cc2ccc(Cl)cc2)C1] |
| 168 | 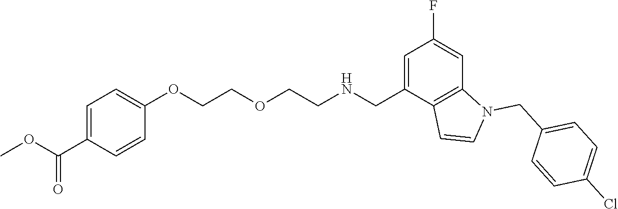 [SMILES: COC(=O)c1ccc(OCCOCCNCc2cc(F)cc3c2ccn3Cc2ccc(Cl)cc2)cc1] |
| 169 | 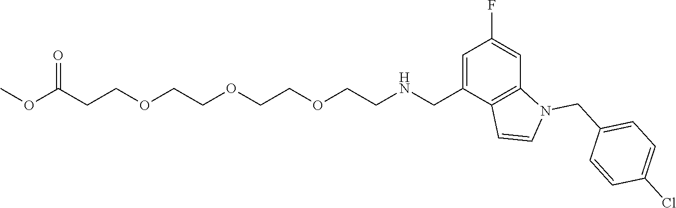 [SMILES: COC(=O)CCOCCOCCOCCNCc1cc(F)cc2c1ccn2Cc1ccc(Cl)cc1] |
| 170 | 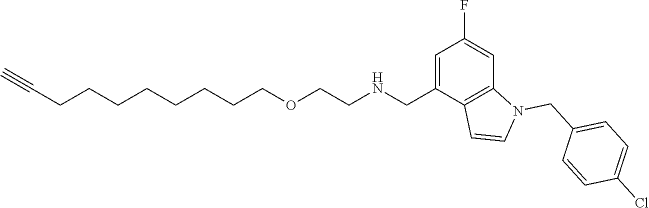 [SMILES: C#CCCCCCCCCOCCNCc1cc(F)cc2c1ccn2Cc1ccc(Cl)cc1] |
| 171 | 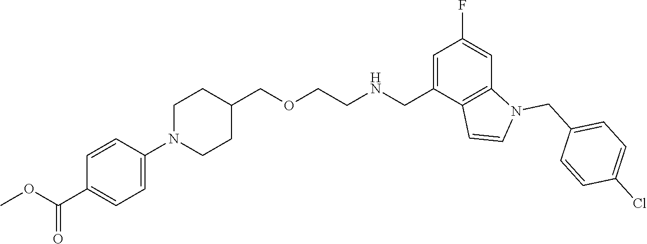 [SMILES: COC(=O)c1ccc(N2CCC(COCCNCc3cc(F)cc4c3ccn4Cc3ccc(Cl)cc3)CC2)cc1] |
| 172 | 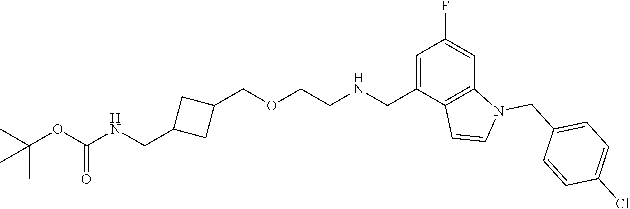 [SMILES: CC(C)(C)OC(=O)NCC1CC(COCCNCc2cc(F)cc3c2ccn3Cc2ccc(Cl)cc2)C1] |
| 173 | 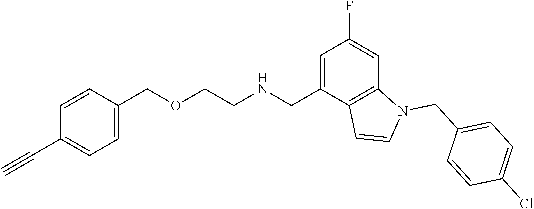 [SMILES: C#Cc1ccc(COCCNCc2cc(F)cc3c2ccn3Cc2ccc(Cl)cc2)cc1] |
| 174 | 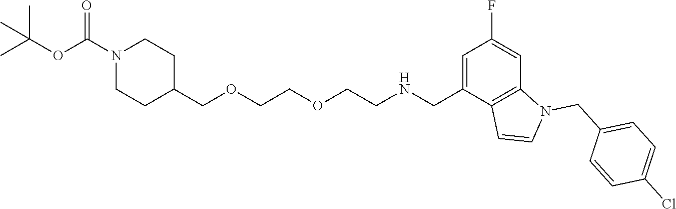 [SMILES: CC(C)(C)OC(=O)N1CCC(COCCOCCNCc2cc(F)cc3c2ccn3Cc2ccc(Cl)cc2)CC1] |
| 175 | 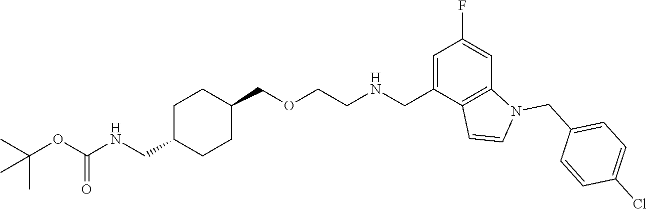 [SMILES: CC(C)(C)OC(=O)NC[C@H]1CC[C@H](COCCNCc2cc(F)cc3c2ccn3Cc2ccc(Cl)cc2)CC1] |
| 176 | 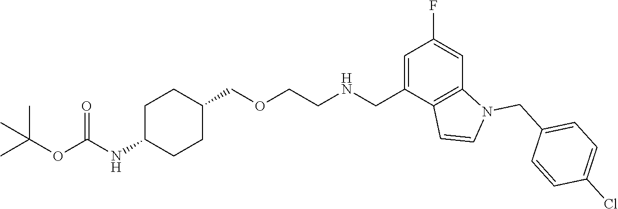 [SMILES: CC(C)(C)OC(=O)N[C@H]1CC[C@@H](COCCNCc2cc(F)cc3c2ccn3Cc2ccc(Cl)cc2)CC1] |

TABle 3
Various compounds of the disclosure (series III)

| Comp. | |
|---|---|
| ID | Structure |
| 97 | 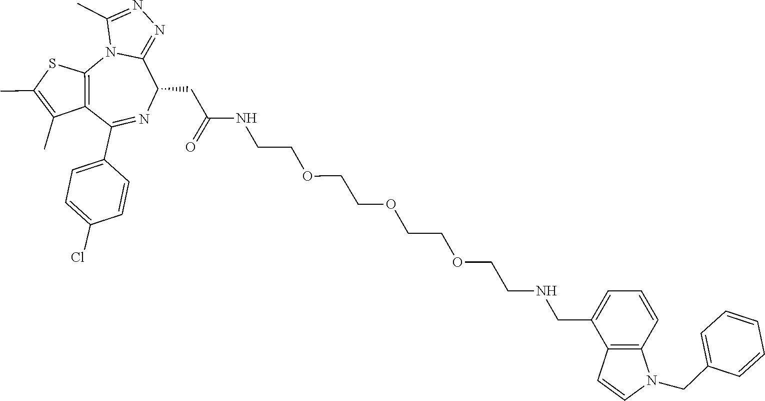 [SMILES: Cc1sc2c(c1C)C(c1ccc(Cl)cc1)=N[C@@H](CC(=O)NCCOCCOCCOCCNCc1cccc3c1ccn3Cc1ccccc1)c1nnc(C)n1-2] |
| 98 | 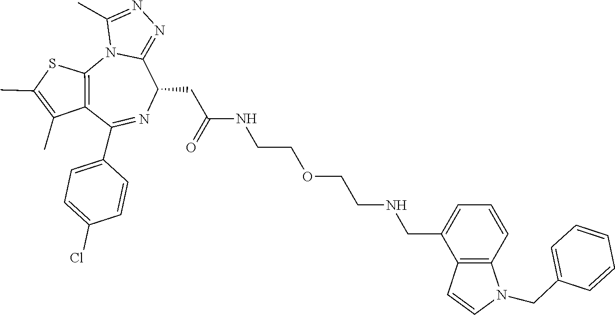 [SMILES: Cc1sc2c(c1C)C(c1ccc(Cl)cc1)=N[C@@H](CC(=O)NCCOCCNCc1cccc3c1ccn3Cc1ccccc1)c1nnc(C)n1-2] |
| 99 | 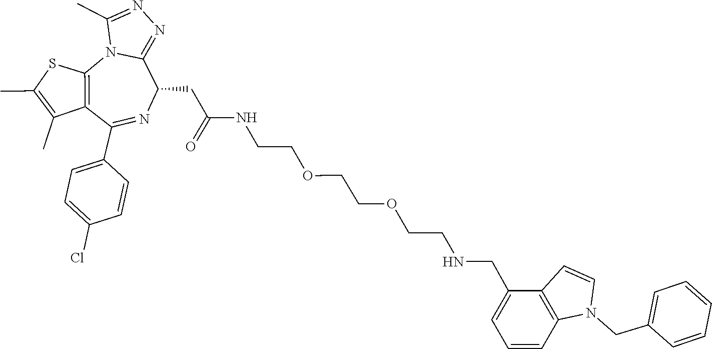 [SMILES: Cc1sc2c(c1C)C(c1ccc(Cl)cc1)=N[C@@H](CC(=O)NCCOCCOCCNCc1cccc3c1ccn3Cc1ccccc1)c1nnc(C)n1-2] |
| 100 | 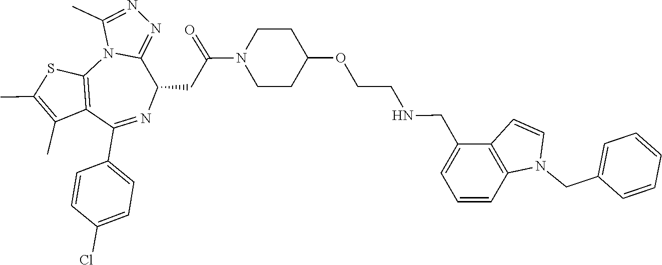 [SMILES: Cc1sc2c(c1C)C(c1ccc(Cl)cc1)=N[C@@H](CC(=O)N1CCC(OCCNCc3cccc4c3ccn4Cc3ccccc3)CC1)c1nnc(C)n1-2] |
| 101 | 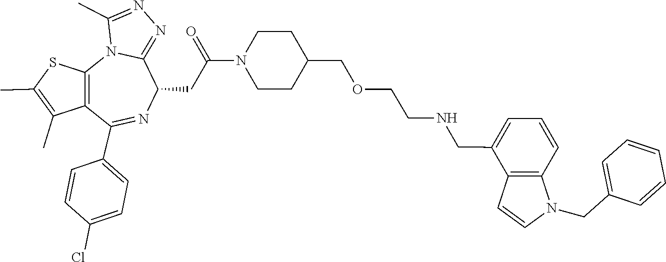 [SMILES: Cc1sc2c(c1C)C(c1ccc(Cl)cc1)=N[C@@H](CC(=O)N1CCC(COCCNCc3cccc4c3ccn4Cc3ccccc3)CC1)c1nnc(C)n1-2] |
| 102 | 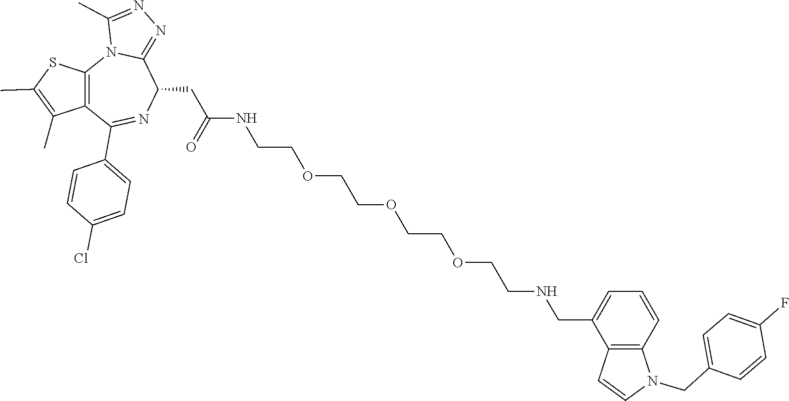 [SMILES: Cc1sc2c(c1C)C(c1ccc(Cl)cc1)=N[C@@H](CC(=O)NCCOCCOCCOCCNCc1cccc3c1ccn3Cc1ccc(F)cc1)c1nnc(C)n1-2] |
| 103 | 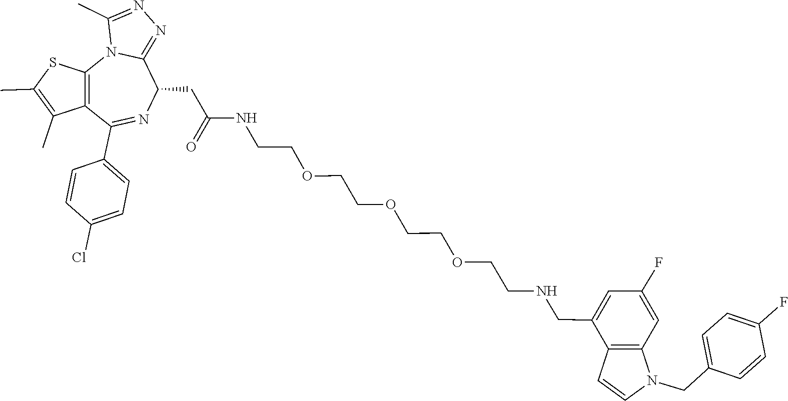 [SMILES: Cc1sc2c(c1C)C(c1ccc(Cl)cc1)=N[C@@H](CC(=O)NCCOCCOCCOCCNCc1cc(F)cc3c1ccn3Cc1ccc(F)cc1)c1nnc(C)n1-2] |
| 104 | 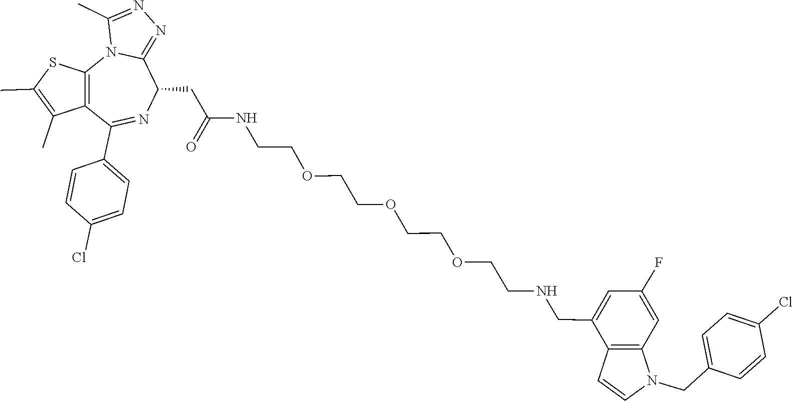 [SMILES: Cc1sc2c(c1C)C(c1ccc(Cl)cc1)=N[C@@H](CC(=O)NCCOCCOCCOCCNCc1cc(F)cc3c1ccn3Cc1ccc(Cl)cc1)c1nnc(C)n1-2] |
| 105 | 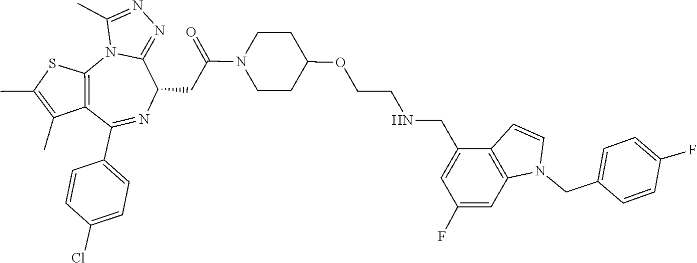 [SMILES: Cc1sc2c(c1C)C(c1ccc(Cl)cc1)=N[C@@H](CC(=O)N1CCC(OCCNCc3cc(F)cc4c3ccn4Cc3ccc(F)cc3)CC1)c1nnc(C)n1-2] |
| 106 | 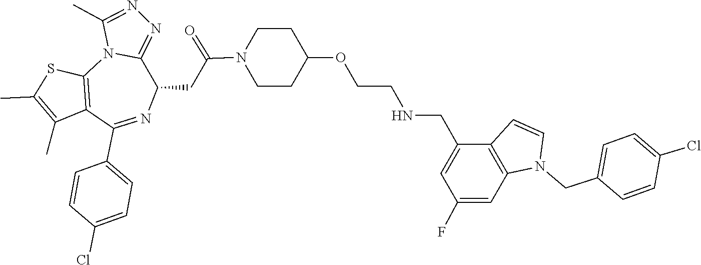 [SMILES: Cc1sc2c(c1C)C(c1ccc(Cl)cc1)=N[C@@H](CC(=O)N1CCC(OCCNCc3cc(F)cc4c3ccn4Cc3ccc(Cl)cc3)CC1)c1nnc(C)n1-2] |
| 107 | 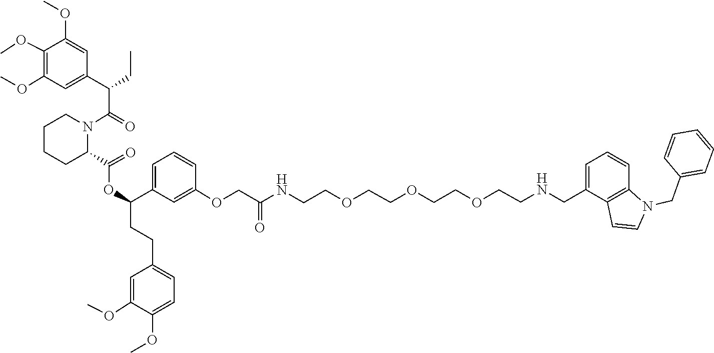 [SMILES: CC[C@H](C(=O)N1CCCC[C@H]1C(=O)O[C@H](CCc1ccc(OC)c(OC)c1)c1cccc(OCC(=O)NCCOCCOCCOCCNCc2cccc3c2ccn3Cc2ccccc2)c1)c1cc(OC)c(OC)c(OC)c1] |
| 177 | 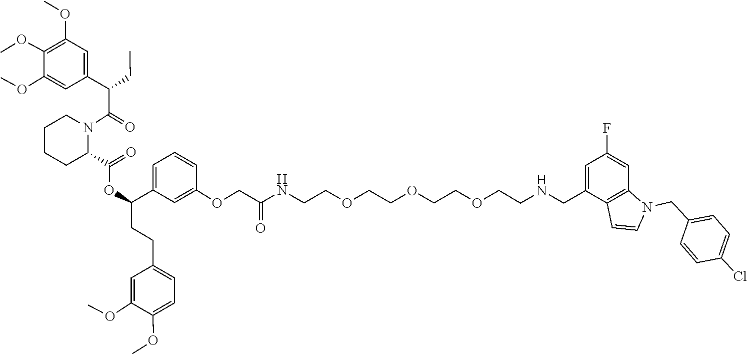 [SMILES: CC[C@H](C(=O)N1CCCC[C@H]1C(=O)O[C@H](CCc1ccc(OC)c(OC)c1)c1cccc(OCC(=O)NCCOCCOCCOCCNCc2cc(F)cc3c2ccn3Cc2ccc(Cl)cc2)c1)c1cc(OC)c(OC)c(OC)c1] |
| 182 | 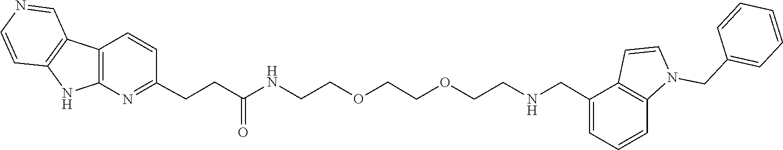 [SMILES: O=C(CCc1ccc2c(n1)[nH]c1ccncc12)NCCOCCOCCNCc1cccc2c1ccn2Cc1ccccc1] |
| 183 | 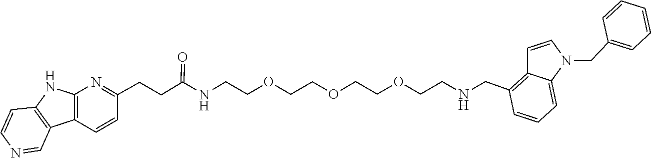 [SMILES: O=C(CCc1ccc2c(n1)[nH]c1ccncc12)NCCOCCOCCOCCNCc1cccc2c1ccn2Cc1ccccc1] |
| 184 | 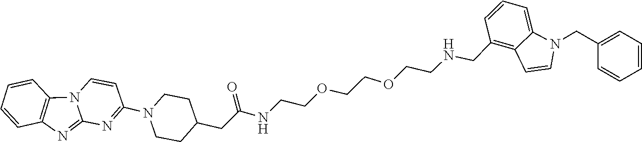 [SMILES: O=C(CC1CCN(c2ccn3c(n2)nc2ccccc23)CC1)NCCOCCOCCNCc1cccc2c1ccn2Cc1ccccc1] |
| 185 | 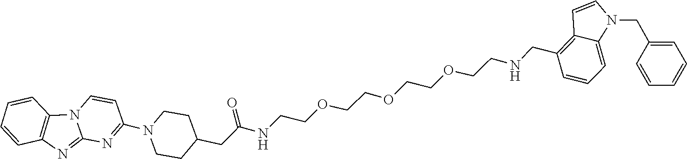 [SMILES: O=C(CC1CCN(c2ccn3c(n2)nc2ccccc23)CC1)NCCOCCOCCOCCNCc1cccc2c1ccn2Cc1ccccc1] |
| 186 | 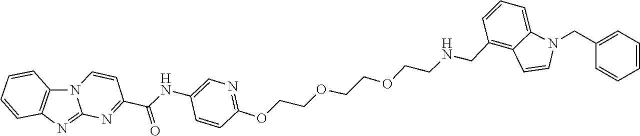 [SMILES: O=C(Nc1ccc(OCCOCCOCCNCc2cccc3c2ccn3Cc2ccccc2)nc1)c1ccn2c(n1)nc1ccccc12] |
| 187 | 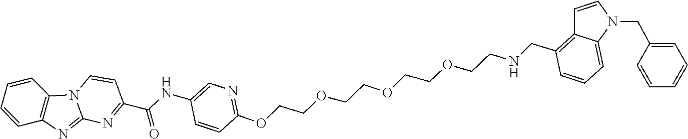 [SMILES: O=C(Nc1ccc(OCCOCCOCCOCCNCc2cccc3c2ccn3Cc2ccccc2)nc1)c1ccn2c(n1)nc1ccccc12] |
| 188 | 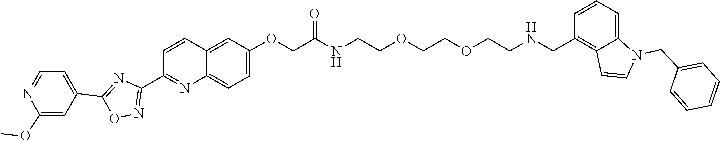 [SMILES: COc1cc(-c2nc(-c3ccc4cc(OCC(=O)NCCOCCOCCNCc5cccc6c5ccn6Cc5ccccc5)ccc4n3)no2)ccn1] |
| 189 | 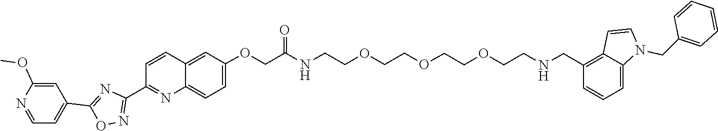 [SMILES: COc1cc(-c2nc(-c3ccc4cc(OCC(=O)NCCOCCOCCOCCNCc5cccc6c5ccn6Cc5ccccc5)ccc4n3)no2)ccn1] |
| 190 | 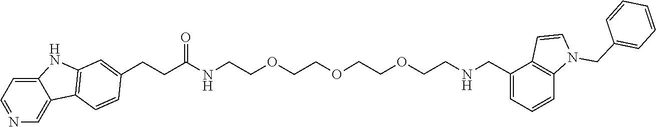 [SMILES: O=C(CCc1ccc2c(c1)[nH]c1ccncc12)NCCOCCOCCOCCNCc1cccc2c1ccn2Cc1ccccc1] |
| 191 | 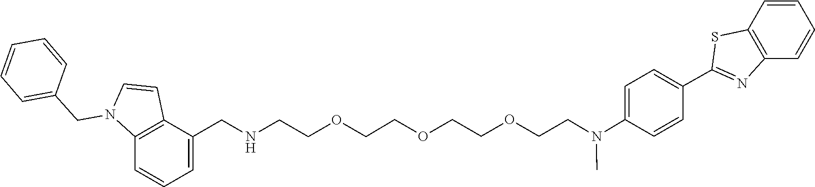 [SMILES: CN(CCOCCOCCOCCNCc1cccc2c1ccn2Cc1ccccc1)c1ccc(-c2nc3ccccc3s2)cc1] |
| 192 | 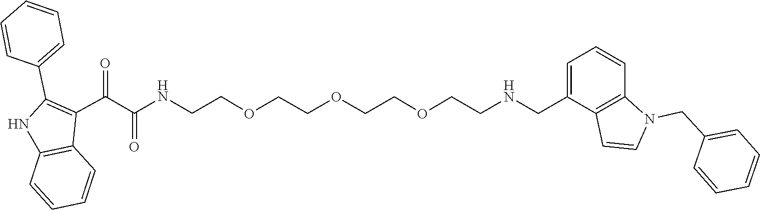 [SMILES: O=C(NCCOCCOCCOCCNCc1cccc2c1ccn2Cc1ccccc1)C(=O)c1c(-c2ccccc2)[nH]c2ccccc12] |
| 193 | 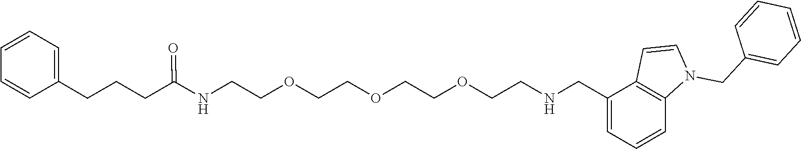 [SMILES: O=C(CCCc1ccccc1)NCCOCCOCCOCCNCc1cccc2c1ccn2Cc1ccccc1] |
| 194 | 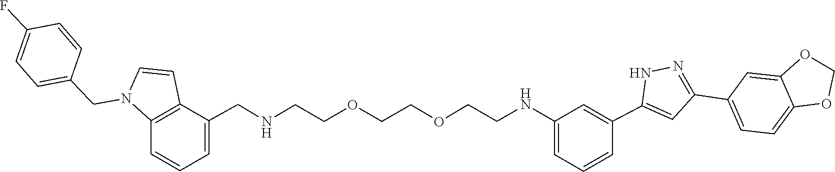 [SMILES: Fc1ccc(Cn2ccc3c(CNCCOCCOCCNc4cccc(-c5cc(-c6ccc7c(c6)OCO7)n[nH]5)c4)cccc32)cc1] |
| 195 | 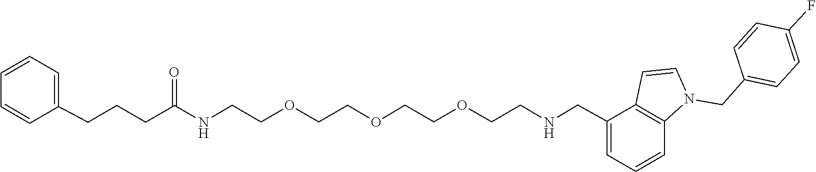 [SMILES: O=C(CCCc1ccccc1)NCCOCCOCCOCCNCc1cccc2c1ccn2Cc1ccc(F)cc1] |
| 196 | 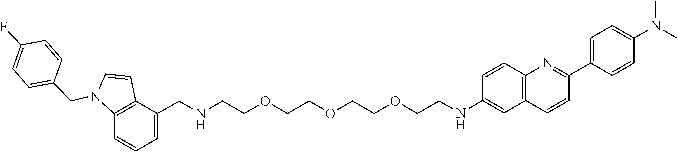 [SMILES: CN(C)c1ccc(-c2ccc3cc(NCCOCCOCCOCCNCc4cccc5c4ccn5Cc4ccc(F)cc4)ccc3n2)cc1] |
| 197 | 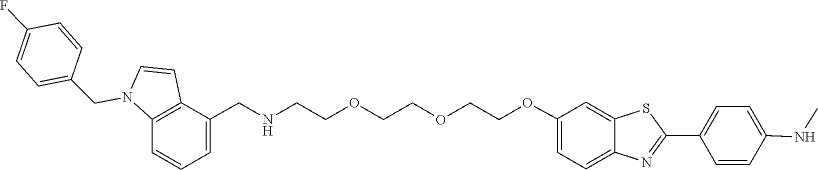 [SMILES: CNc1ccc(-c2nc3ccc(OCCOCCOCCNCc4cccc5c4ccn5Cc4ccc(F)cc4)cc3s2)cc1] |
| 198 | 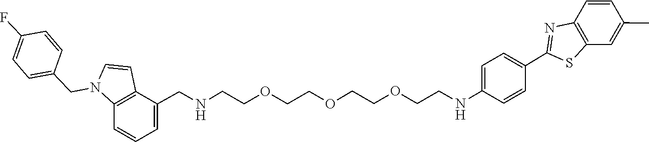 [SMILES: Cc1ccc2nc(-c3ccc(NCCOCCOCCOCCNCc4cccc5c4ccn5Cc4ccc(F)cc4)cc3)sc2c1] |
| 199 | 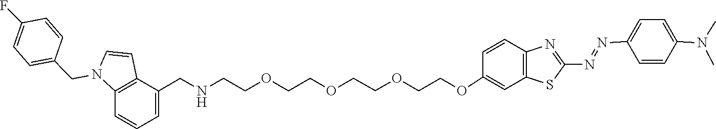 [SMILES: CN(C)c1ccc(/N=N/c2nc3ccc(OCCOCCOCCOCCNCc4cccc5c4ccn5Cc4ccc(F)cc4)cc3s2)cc1] |
| 200 | 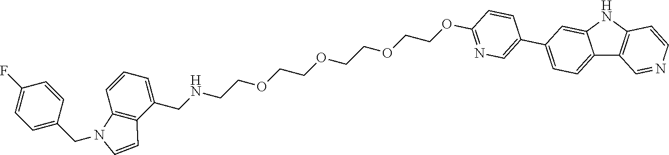 [SMILES: Fc1ccc(Cn2ccc3c(CNCCOCCOCCOCCOc4ccc(-c5ccc6c(c5)[nH]c5ccncc56)cn4)cccc32)cc1] |
| 201 | 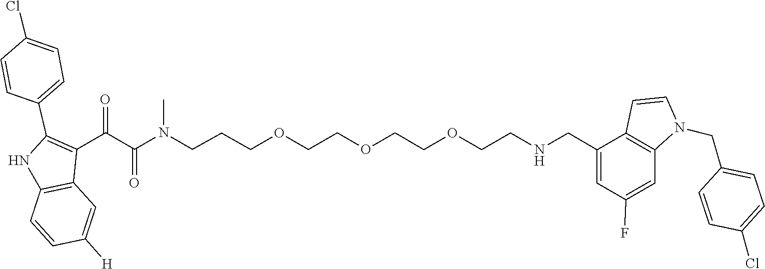 [SMILES: CN(CCCOCCOCCOCCNCc1cc(F)cc2c1ccn2Cc1ccc(Cl)cc1)C(=O)C(=O)c1c(-c2ccc(Cl)cc2)[nH]c2ccccc12] |
| 202 | 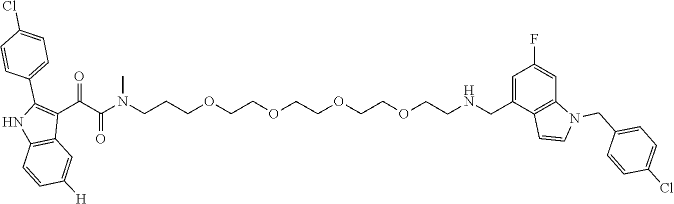 [SMILES: CN(CCCOCCOCCOCCOCCNCc1cc(F)cc2c1ccn2Cc1ccc(Cl)cc1)C(=O)C(=O)c1c(-c2ccc(Cl)cc2)[nH]c2ccccc12] |
| 203 | 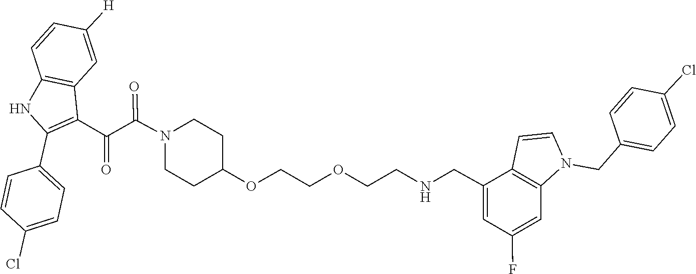 [SMILES: O=C(C(=O)N1CCC(OCCOCCNCc2cc(F)cc3c2ccn3Cc2ccc(Cl)cc2)CC1)c1c(-c2ccc(Cl)cc2)[nH]c2ccccc12] |
| 204 | 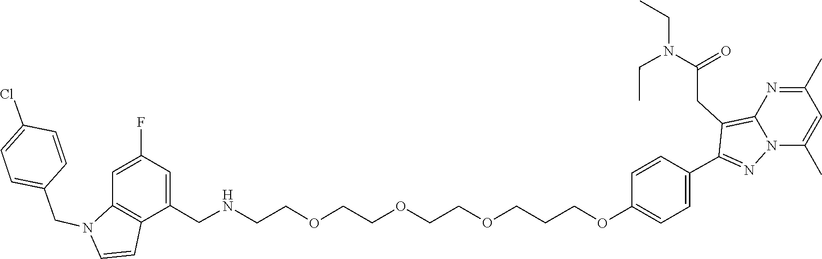 [SMILES: CCN(CC)C(=O)Cc1c(-c2ccc(OCCCOCCOCCOCCNCc3cc(F)cc4c3ccn4Cc3ccc(Cl)cc3)cc2)nn2c(C)cc(C)nc12] |
| 205 | 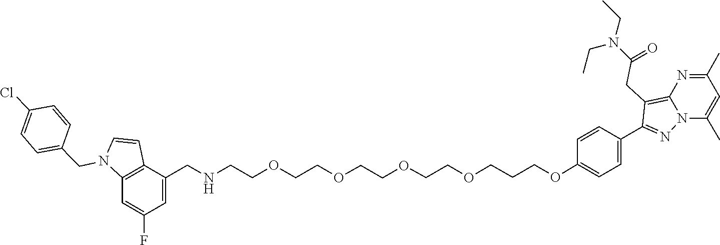 [SMILES: CCN(CC)C(=O)Cc1c(-c2ccc(OCCCOCCOCCOCCOCCNCc3cc(F)cc4c3ccn4Cc3ccc(Cl)cc3)cc2)nn2c(C)cc(C)nc12] |
| 206 | 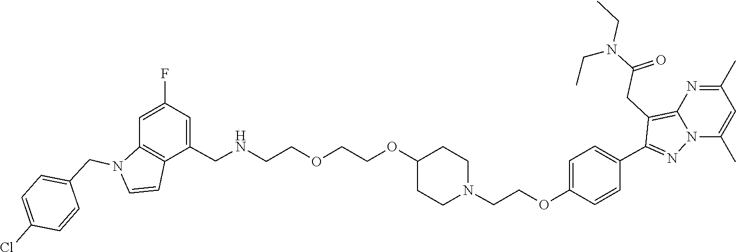 [SMILES: CCN(CC)C(=O)Cc1c(-c2ccc(OCCN3CCC(OCCOCCNCc4cc(F)cc5c4ccn5Cc4ccc(Cl)cc4)CC3)cc2)nn2c(C)cc(C)nc12] |

### Compositions

The compounds of the present disclosure (e.g., a compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or Table 1, 2, or 3, or a pharmaceutically acceptable salt, stereoisomer, or deuterated form thereof) may be used on their own but will generally be administered in the form of a pharmaceutical composition in which the compound (active ingredient) is in association with pharmaceutically acceptable adjuvant(s), diluents(s), or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2nd Ed. 2002.

In some embodiments, the present disclosure provides a pharmaceutical composition comprising one or more compounds of the present disclosure (e.g., a compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (percent (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or Table 1, 2, or 3) or a pharmaceutically acceptable salt, stereoisomer, or deuterated form thereof.

Depending on the mode of administration, the pharmaceutical composition can comprise from 0.01 to 99% wt (percent by weight), from 0.05 to 95% wt, from 0.1 to 90% wt, from 0.2 to 80% wt, from 0.3 to 70% wt, from 0.4 to 60% wt, or from 0.5 to 50% wt, of active ingredient (e.g., a compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or Table 1, 2, or 3, or a pharmaceutically acceptable salt, stereoisomer, or deuterated form thereof), all percentages by weight being based on total composition.

In embodiments, the present disclosure provides pharmaceutical composition(s) comprising one or more compounds of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or Table 1, 2, or 3, or a pharmaceutically acceptable salt, stereoisomer, or deuterated form thereof, and pharmaceutically acceptable adjuvant(s), diluent(s) or carrier(s).

In some embodiments, the present disclosure provides pharmaceutical composition(s) comprising one or more compounds of Table 1, 2, or 3, or a pharmaceutically acceptable salt, stereoisomer, or deuterated form thereof, and pharmaceutically acceptable adjuvant(s), diluent(s) or carrier(s).

The pharmaceutically acceptable excipients and adjuvants are added to the composition or formulation for a variety of purposes. In some embodiments, a pharmaceutical composition comprising one or more compounds disclosed herein, or a pharmaceutically acceptable salt, stereoisomer, or deuterated form thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, transdermally, buccally, sublingually, or rectally in formulations containing pharmaceutically acceptable carriers, adjuvants, and vehicles. The term parenteral as used here includes subcutaneous, intravenous, intramuscular, and intraarterial injections with a variety of infusion techniques. Intraarterial and intravenous injection as used herein includes administration through catheters.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion.

In some embodiments, the pharmaceutical composition is formulated for inhalation. The inhalable formulations can be, for example, formulated as a nasal spray, dry powder, or an aerosol administrable through a metered-dose inhaler.

### Therapeutic Use

In embodiments, the compounds of the present disclosure are p62 modulators, and thus may be used in any disease area where p62 plays a role. In embodiments, the compounds of the present disclosure are NBR1 modulators, and thus may be used in any disease area where NBR1 plays a role. As such, in one aspect of the disclosure, a method of treatment is provided. The method of treatment, in one embodiment, comprises, administering to a subject in need thereof, a composition comprising an effective amount of a compound of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or Table 1, 2, or 3, or a pharmaceutically acceptable salt, stereoisomer, or deuterated form thereof. In embodiments, the composition is administered to the patient for an administration period.

In embodiments, a compound or composition of the present disclosure is used in a method for modulating autophagy. In some embodiments, the present disclosure provides methods of modulating autophagy. In some embodiments, the method comprises contacting p62 (e.g., one or more amino acids present in p62 protein) with compounds or compositions of the present disclosure (e.g., compounds of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or Table 1, 2, or 3, or a pharmaceutically acceptable salt, stereoisomer, or deuterated form thereof). In some embodiments, the compound increases activity of p62, thereby causing autophagy. In some embodiments, the compound decreases activity of p62, thereby reducing autophagy.

In some embodiments, the method comprises contacting NBR1 (e.g., one or more amino acids present in NBR1 protein) with compounds or compositions of the present disclosure (e.g., compounds of formula (I), (I-A), (I-B), (I-C), (I-D), (I-E), (I-F), (I-G), (I-A-1), (I-A-2), (I-A-3), (I-B-1), (I-C-1), (I-D-1), (I-E-1), (I-F-1), (I-G-1), (I-A-1-a), (I-A-2-a), (I-A-3-a), (I-B-1-a), (I-C-1-a), (I-D-1-a), (I-E-1-a), (I-F-1-a), (I-G-1-a), (II), (II-A), (II-B), (II-C), (II-D), (II-E), (II-F), (II-G), (II-A-1), (II-A-2), (II-A-3), (II-B3-1), (II-C-1), (II-D-1), (II-E-1), (II-F-1), (II-G-1), (III), (III-A), (III-B), (III-C), (III-D), (III-E), (III-F), (III-G), (III-A-1), (III-A-2), (III-A-3), (III-B-1), (III-C-1), (III-D-1), (III-E-1), (III-F-1), or (III-G-1), or Table 1, 2, or 3, or a pharmaceutically acceptable salt, stereoisomer, or deuterated form thereof). In some embodiments, the compound increases activity of NBR1, thereby causing autophagy. In some embodiments, the compound decreases activity of NBR1, thereby reducing autophagy.

In embodiments, a compound or composition of the present disclosure is administered to a subject in need thereof in a method for inducing autophagy. In some embodiments, the compound contacts p62, thereby inducing autophagy.

In embodiments, a compound or composition of the present disclosure is administered to a subject in need thereof in a method for inducing autophagy. In some embodiments, the compound contacts NBR1, thereby inducing autophagy.

In embodiments, a compound or composition of the present disclosure is administered to a subject in need thereof in a method for degrading target proteins, protein aggregates, protein complexes, lipids (e.g., lipid droplets), bacteria, or viruses. In some embodiments, a compound or composition of the present disclosure is administered to a subject in need thereof in a method for reducing the quantity of target proteins, protein aggregates, protein complexes, lipids, bacteria, or viruses.

In embodiments, a compound or composition of the present disclosure is administered to a subject in need thereof in a method for degrading a target protein. In some embodiments, a compound or composition of the present disclosure is administered to a subject in need thereof in a method for reducing the quantity of target proteins.

In embodiments, a compound or composition of the present disclosure is administered to a patient in a method for treating cancer (e.g., cancer metastasis), metabolic diseases, inflammation, neurodegenerative disorders, and infectious diseases.

In some embodiments, the cancer is a breast cancer, colorectal cancer, kidney cancer, ovarian cancer, gastric cancer, thyroid cancer, urothelial cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, hematologic cancer, sarcoma, gastrointestinal stromal tumor, peripheral nerve sheath tumor, myeloma, mesothelioma, endometrial cancer, and/or leukemia.

In some embodiments, the cancer is breast cancer (e.g., ER negative breast cancer, triple negative breast cancer, basal-like breast cancers, HER2-positive breast cancers), kidney cancer (e.g., renal cell carcinoma (RCC)), prostate cancer, glioblastoma, or leukemia (e.g., chronic myelogenous leukemia, acute myelogenous leukemia, acute lymphoblastic leukemia).

In some embodiments, the neurodegenerative disorder is Alzheimer's disease, Parkinson's disease, and/or Huntington's Disease.

In some embodiments, the infectious disease is a bacterial infection and/or a virus infection.

### Numbered Embodiments of the Disclosure

In addition to the disclosure above, the Examples below, and the appended claims, the disclosure sets forth the following numbered embodiments.

1. A compound of formula (I)

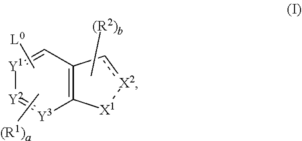

or a pharmaceutically acceptable salt, stereoisomer, or deuterated form thereof,
wherein:
   L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R^{A} is independently H, C₁₋₆ alkyl, C₁₋₆ haloalkyl, C₃₋₈ cycloalkyl, C₃₋₈ hydroxycycloalkyl, aryl, or heterocycle; each R^{B} is independently C₁₋₆ hydroxyalkyl, C₁₋₆ alkoxy, C₁₋₆ alkylene-NH₂, or C₁₋₆ alkylene-SH;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

2. The compound of embodiment 1, having a structure of formula (I-A):

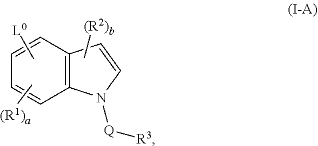

or a pharmaceutically acceptable salt, a stereoisomer, or a deuterated form thereof.

3. The compound of embodiment 1, having a structure of formula (I-B), (I-C), or (I-D):

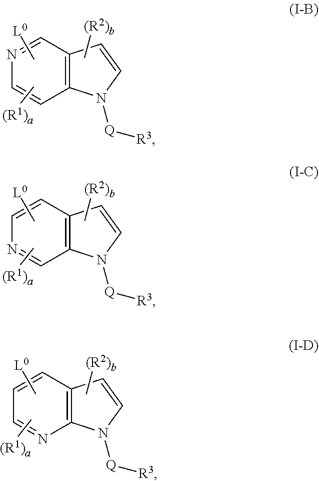

or a pharmaceutically acceptable salt, a stereoisomer, or a deuterated form thereof.

4. The compound of embodiment 1, having a structure of formula (I-E), (I-F), or (I-G):

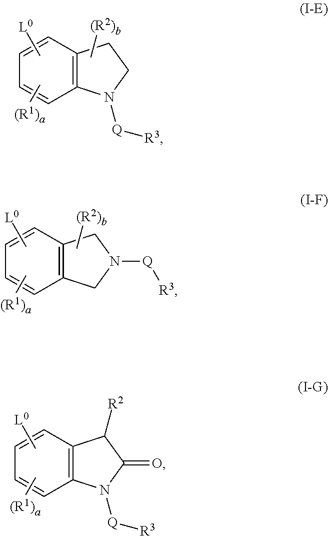

or a pharmaceutically acceptable salt, a stereoisomer, or a deuterated form thereof.

5. The compound of embodiment 1 or 2, having a structure of formula (I-A-1), (I-A-2), or (I-A-3):

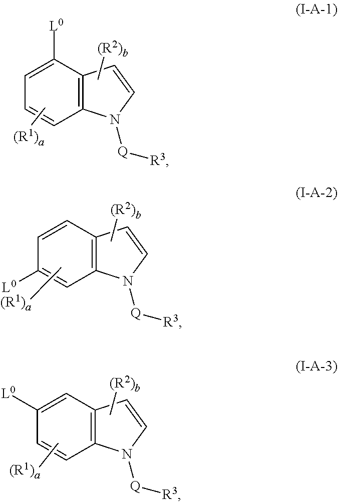

or a pharmaceutically acceptable salt, a stereoisomer, or a deuterated form thereof.

6. The compound of embodiment 1 or 3, having a structure of formula (I-B-1), (I-C-1), or (I-D-1):

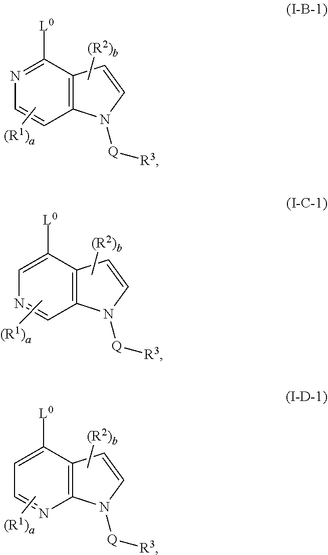

or a pharmaceutically acceptable salt, a stereoisomer, or a deuterated form thereof.

7. The compound of embodiment 1 or 4, having a structure of formula (I-E-1), (I-F-1), or (I-G-1):

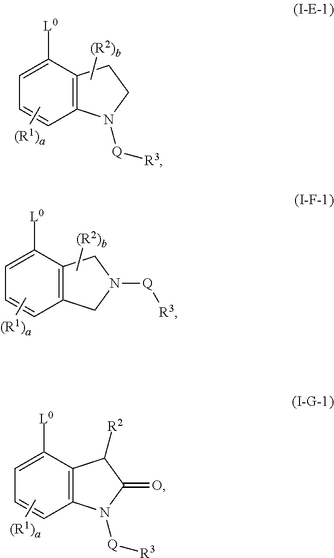

or a pharmaceutically acceptable salt, a stereoisomer, or a deuterated form thereof.

8. The compound of any one of embodiments 1-7, or a pharmaceutically acceptable salt, stereoisomer, or deuterated form thereof, wherein L⁰ is —(C₁₋₆ alkylene)-N(H)(C₁₋₆ hydroxyalkyl), —(C₁₋₆ alkylene)-N(C₁₋₆ alkyl)(C₁₋₆ hydroxyalkyl), —(C₁₋₆ alkylene)-N(H)(C₁₋₆ alkoxy), —(C₁₋₆ alkylene)-N(C₁₋₆ alkyl)(C₁₋₆ alkoxy), —(C₁₋₆ alkylene)-N(H)C(O)(C₁₋₆ alkoxy), —(C₁₋₆ alkylene)-N(C₁₋₆ alkyl)C(O)(C₁₋₆ alkoxy), —(C₁₋₆ alkylene)-N(H)C(O)N(H)(C₁₋₆ alkoxy), —(C₁₋₆ alkylene)-N(C₁₋₆ alkyl)C(O)N(C₁₋₆ alkyl)(C₁₋₆ alkoxy), —(C₃₋₈ cycloalkylene)N(H)(C₁₋₆ hydroxyalkyl), —(C₃₋₈ cycloalkylene)N(C₁₋₆ alkyl)(C₁₋₆ hydroxyalkyl), —O—(C₁₋₆ alkylene)-N(H)(C₁₋₆ hydroxyalkyl), —O—(C₁₋₆ alkylene)-N(C₁₋₆ alkyl)(C₁₋₆ hydroxyalkyl), —N(H)(C₁₋₆ hydroxyalkyl), —N(C₁₋₆ alkyl)(C₁₋₆ hydroxyalkyl), or —N(H)C(O)(C₁₋₆ hydroxyalkyl), —N(C₁₋₆ alkyl)C(O)(C₁₋₆ hydroxyalkyl),
wherein the alkylene or alkyl is optionally substituted with —OH or halogen.

9. The compound of any one of embodiments 1-8, or a pharmaceutically acceptable salt, stereoisomer, or deuterated form thereof, wherein L⁰ is
—CH₂NHCH₂CH₂OH,
—CH₂NHCH₂CH(CH₃)OH,

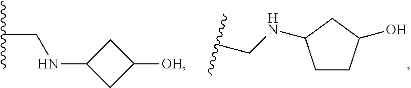 [SMILES: *CNC1CC(O)C1 ; *CNC1CCC(O)C1]

—CH₂NHCH₂CH₂OCH(CH₃)₂,
—CH₂OCH₂CH₂NHCH₃,
—CH₂OCH₂CH₂NHCH(CH₃)₂,
—C(O)NHCH₂CH₂OH,
—NHC(O)CH₂CH₂OH,
—NHC(O)CH₂OH,
—CH₂NHC(O)NHCH₂CH₂OH,

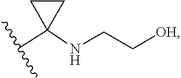 [SMILES: *C1(NCCO)CC1]

—NHC(O)NHCH₂CH₂OH,
—OCH₂CH(OH)CH₂NHCH(CH₃)₂,
—OCH₂CH₂C(O)NHCH₃,
—CH₂NHC(O)CH₂OH,
—CH₂NHC(O)CH₂CH₂OH, or
—NHC(O)NHCH₂CH₂OCH₃.

10. The compound of any one of embodiments 1-9, or a pharmaceutically acceptable salt, stereoisomer, or deuterated form thereof, wherein L⁰ is —CH₂NHCH₂CH₂OH, —CH₂NHCH₂CH(CH₃)OH, —CH₂OCH₂CH₂NHCH₃, or —OCH₂CH(OH)CH₂NHCH(CH₃)₂.

11. The compound of any one of embodiments 1, 2, 5, and 10, having a structure of formula (I-A-1-a), (I-A-2-a), or (I-A-3-a):

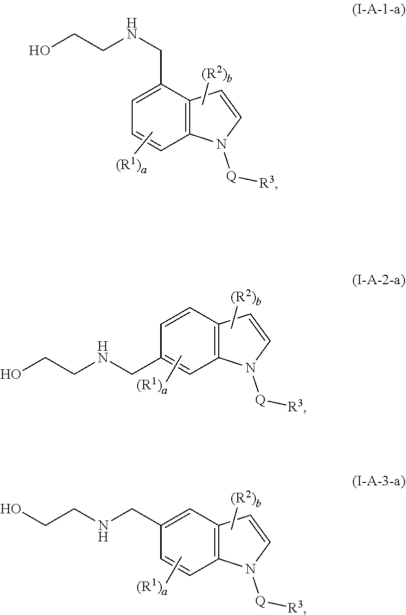

or a pharmaceutically acceptable salt, a stereoisomer, or a deuterated form thereof.

12. The compound of any one of embodiments 1, 3, 6, and 10, having a structure of formula (I-B-1-a), (I-C-1-a), or (I-D-1-a):

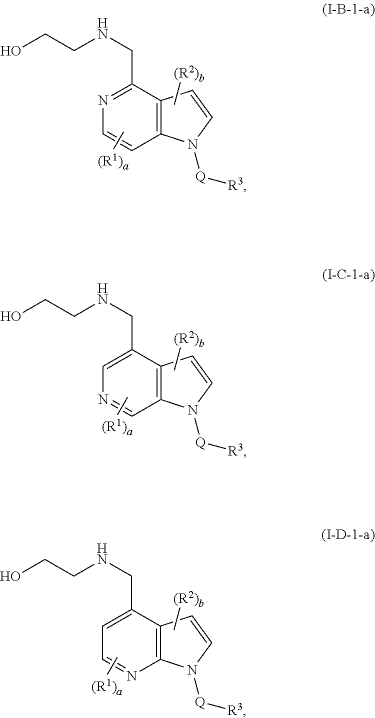

or a pharmaceutically acceptable salt, a stereoisomer, or a deuterated form thereof.

13. The compound of any one of embodiments 1, 4, 7, and 10, having a structure of formula (I-E-1-a), (I-F-1-a), or (I-G-1-a):

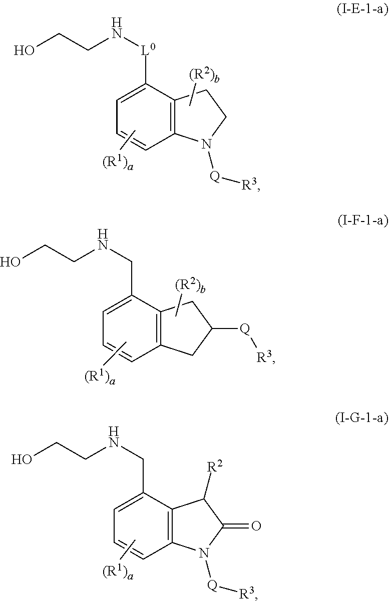

or a pharmaceutically acceptable salt, a stereoisomer, or a deuterated form thereof.

14. The compound of any one of the preceding embodiments, wherein at least one H in L⁰ is replaced by conjugate comprising a ligand that binds to a protein, a protein aggregate, a protein complex, or a lipid.

15. A compound of formula (II)

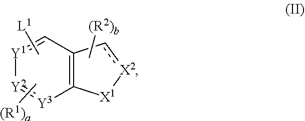

or a pharmaceutically acceptable salt, stereoisomer, or deuterated form thereof,
wherein:
   L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   each R⁵ is independently a bond, a leaving group, a protecting group, H, alkynyl, aryl, or heteroaryl;
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

16. The compound of embodiment 15, or a pharmaceutically acceptable salt, a stereoisomer, or a deuterated form thereof, wherein the compound has a structure of formula (II-A):

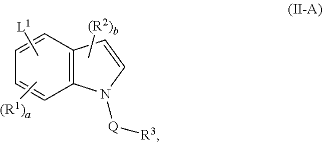

or a pharmaceutically acceptable salt, a stereoisomer, or a deuterated form thereof.

17. The compound of embodiment 15, or a pharmaceutically acceptable salt, a stereoisomer, or a deuterated form thereof, wherein the compound has a structure of formula (II-B), (II-C), or (II-D):

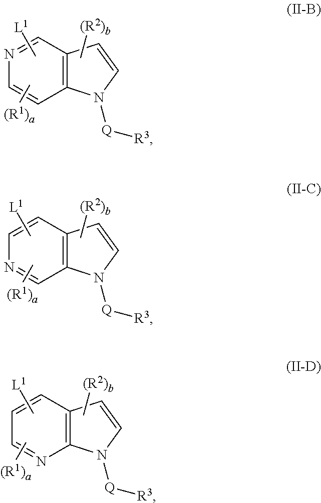

or a pharmaceutically acceptable salt, a stereoisomer, or a deuterated form thereof.

18. The compound of embodiment 15, or a pharmaceutically acceptable salt, a stereoisomer, or a deuterated form thereof, wherein the compound has a structure of formula (II-E), (II-F), or (II-G):

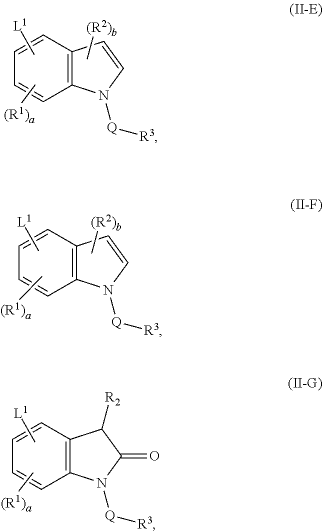

or a pharmaceutically acceptable salt, a stereoisomer, or a deuterated form thereof.

19. The compound of embodiment 16, or a pharmaceutically acceptable salt, a stereoisomer, or a deuterated form thereof, wherein the compound has a structure of formula (II-A-1), (II-A-2), or (II-A-3):

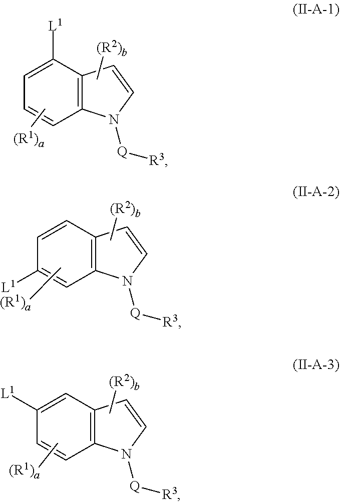

or a pharmaceutically acceptable salt, a stereoisomer, or a deuterated form thereof.

20. The compound of embodiment 17, or a pharmaceutically acceptable salt, a stereoisomer, or a deuterated form thereof, wherein the compound has a structure of formula (II-B-1), (II-C-1), or (II-D-1):

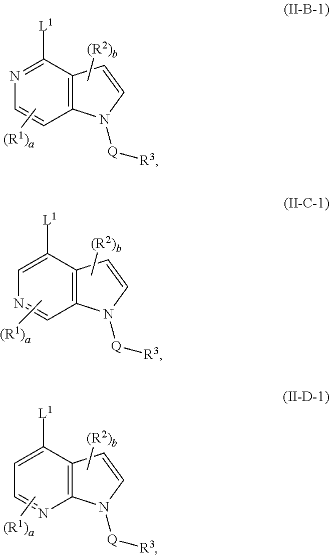

or a pharmaceutically acceptable salt, a stereoisomer, or a deuterated form thereof.

21. The compound of embodiment 18, or a pharmaceutically acceptable salt, a stereoisomer, or a deuterated form thereof, wherein the compound has a structure of formula (II-E-1), (II-F-1), or (II-G-1):

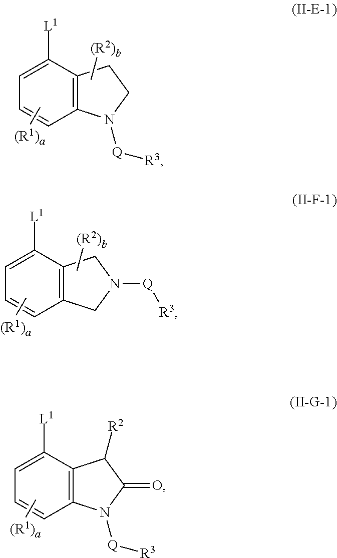

or a pharmaceutically acceptable salt, a stereoisomer, or a deuterated form thereof.

22. The compound of any one of embodiments 15-21, or a pharmaceutically acceptable salt, a stereoisomer, or a deuterated form thereof, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —N(H)C(O)—, —N(C₁-C₆ alkyl)C(O)—, —N(C₃-C₈ cycloalkyl)C(O)—, —C(O)N(H)—, —C(O)N(C₁-C₆ alkyl)-, —C(O)N(C₃-C₈ cycloalkyl)-, arylene, heteroarylene, heterocyclylene, or C₃-C₈ cycloalkylene, wherein each arylene or heteroarylene is optionally and independently substituted with 1 or 2 R^{Z}.

23. The compound of any one of embodiments 15-22, or a pharmaceutically acceptable salt, a stereoisomer, or a deuterated form thereof, wherein L¹ is:
—(C₁₋₆ alkylene)-NH(CH₂CH₂O)ₘ—(CH₂)ₙ—C≡CH,
—(C₁₋₆ alkylene)-NH(CH₂CH₂O)ₘ—(CH₂)ₙ—NH—C(O)O—(C₁₋₆ alkyl),
—(C₁₋₆ alkylene)-NH(CH₂CH₂O)ₘ—(CH₂)ₙ—N(C₁₋₆ alkyl)-C(O)O—(C₁₋₆ alkyl),
—(C₁₋₆ alkylene)-NH(CH₂CH₂O)ₘ—(CH₂)—HET^{A}
—(C₁₋₆ alkylene)-NH(CH₂CH₂O)ₘ—(CH₂)ₙ—NH—(C₁₋₆ alkyl),
—(C₁₋₆ alkylene)-NH(CH₂CH₂O)ₘ—(CH₂)ₙ—C(O)O—(C₁₋₆ alkyl), or
—(C₁₋₆ alkylene)-NH(CH₂CH₂O)ₘ—(CH₂)ₙ—O—C₆₋₁₀ aryl;
wherein HET^{A} is a 4-10 membered heterocycle containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl;
m is an integer of 1-12; and
n is an integer of 0-6.

24. The compound of embodiment 23, or a pharmaceutically acceptable salt, a stereoisomer, or a deuterated form thereof, wherein HET^{A} is piperidinyl or piperazinyl optionally substituted with —C(O)—(C₁₋₆ alkyl), —C(O)O—(C₁₋₆ alkyl), or —(C₁₋₆ alkyl)-O—C₆₋₁₀ aryl.

25. The compound of embodiment 23 or 24, or a pharmaceutically acceptable salt, a stereoisomer, or a deuterated form thereof, wherein m is an integer of 1-6.

26. The compound of embodiment 25, or a pharmaceutically acceptable salt, a stereoisomer, or a deuterated form thereof, wherein m is an integer of 1-4.

27. The compound of any one of embodiments 23-26, or a pharmaceutically acceptable salt, a stereoisomer, or a deuterated form thereof, wherein n is an integer of 0-3.

28. The compound of embodiment 23, or a pharmaceutically acceptable salt, a stereoisomer, or a deuterated form thereof, wherein L¹ is
—CH₂NH—(CH₂CH₂O)₂—(CH₂)₂—C≡CH,
—CH₂NH—(CH₂CH₂O)₃—(CH₂)₂—C≡CH,
—CH₂NH—(CH₂CH₂O)₃—(CH₂)₂—NHC(O)OC(CH₃)₃,
—CH₂NH—(CH₂CH₂O)₃—(CH₂)₃—NCH₃C(O)OC(CH₃)₃,
—CH₂NH—(CH₂CH₂O)₄—(CH₂)₃—NCH₃C(O)OC(CH₃)₃,

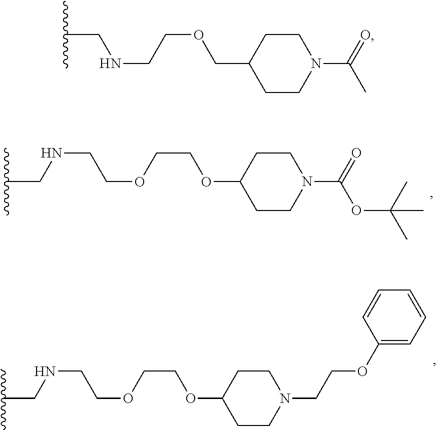 [SMILES: *CNCCOCC1CCN(C(C)=O)CC1 ; *CNCCOCCOC1CCN(C(=O)OC(C)(C)C)CC1 ; *CNCCOCCOC1CCN(CCOc2ccccc2)CC1]

—CH₂NH—(CH₂CH₂O)₂—(CH₂)₂—NHCH₃,
—CH₂NH—(CH₂CH₂O)₃—(CH₂)₂—NHCH₃,
—CH₂NH—(CH₂CH₂O)₂—(CH₂)₂—C(O)OCH₃,

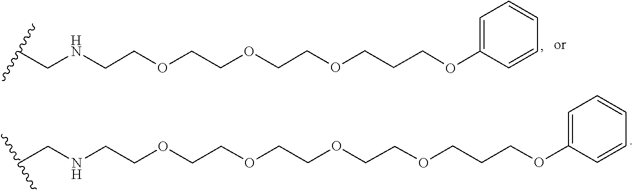 [SMILES: *CNCCOCCOCCOCCCOc1ccccc1 ; *CNCCOCCOCCOCCOCCCOc1ccccc1]

29. The compound of any one of embodiments 15-22, or a pharmaceutically acceptable salt, a stereoisomer, or a deuterated form thereof, wherein L¹ comprises a bivalent moiety selected from:

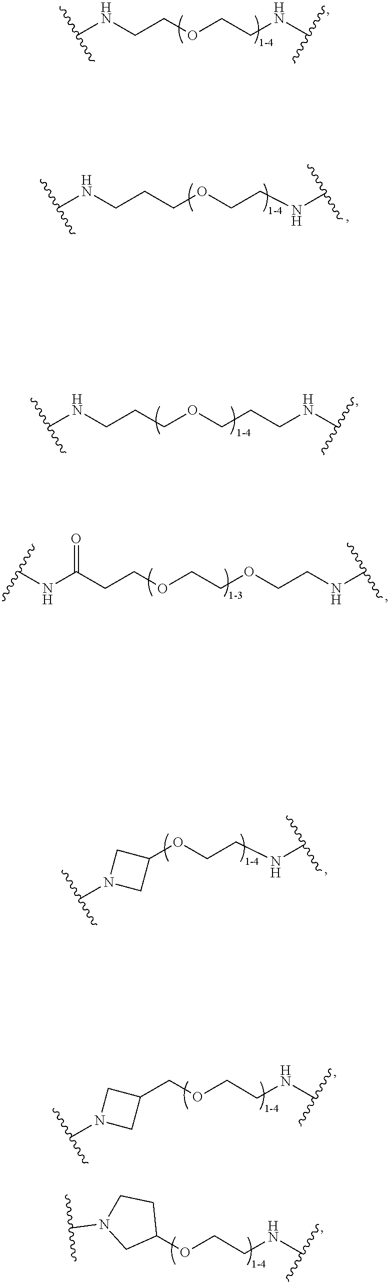

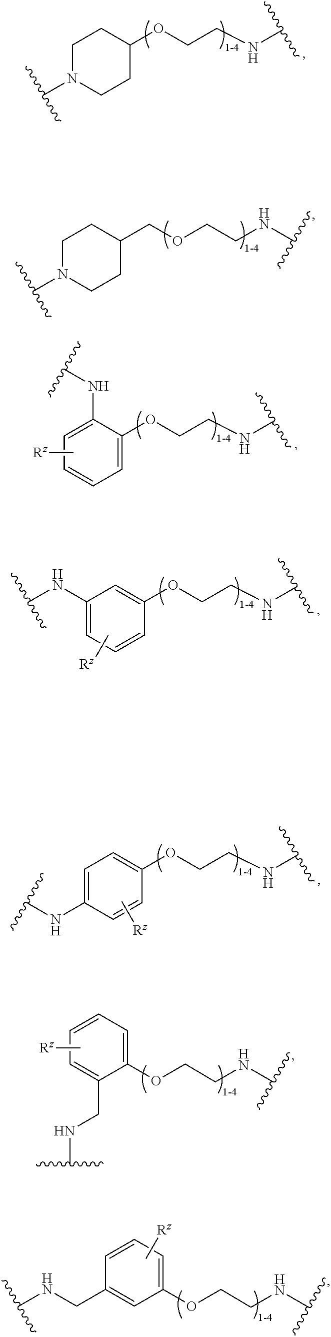

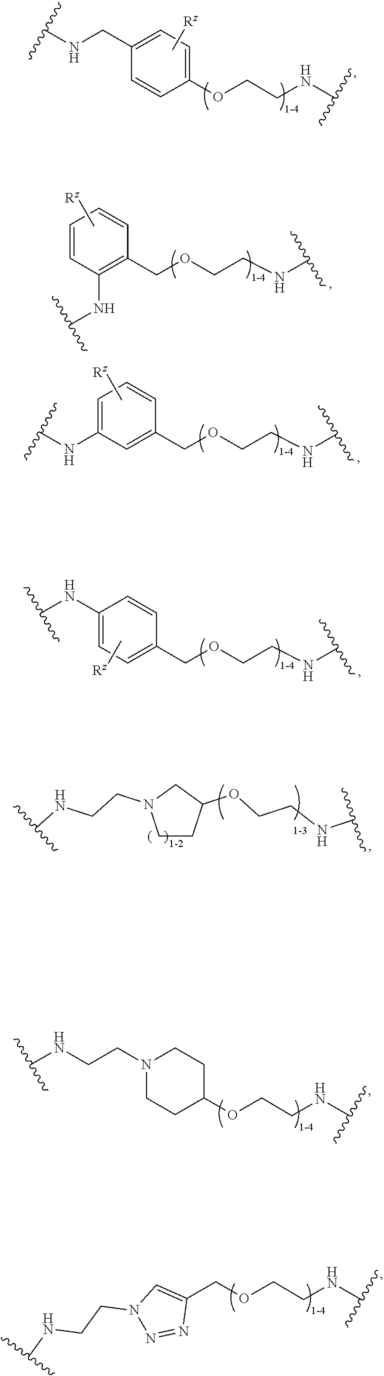

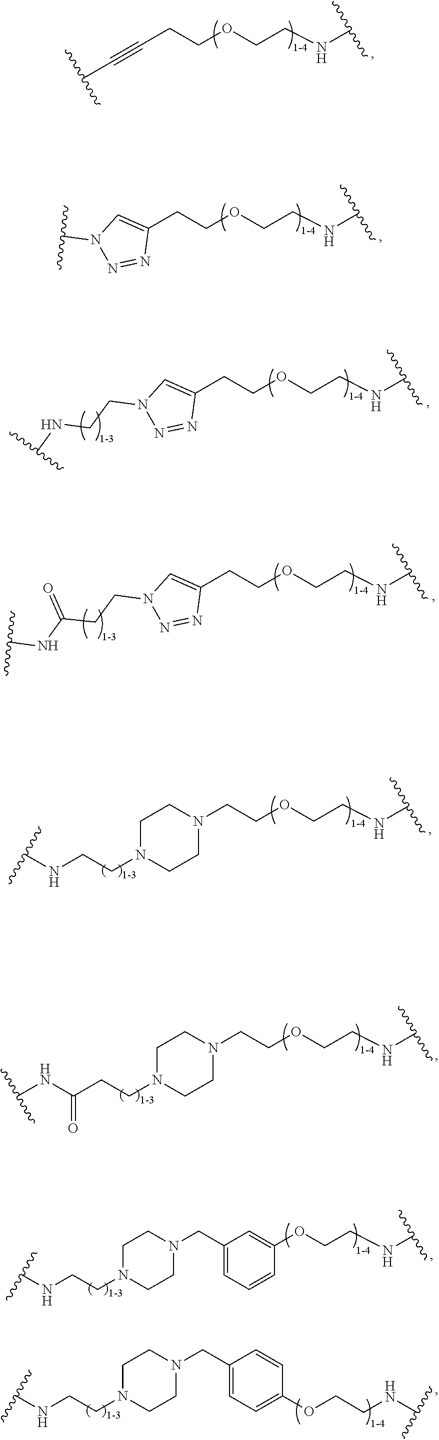

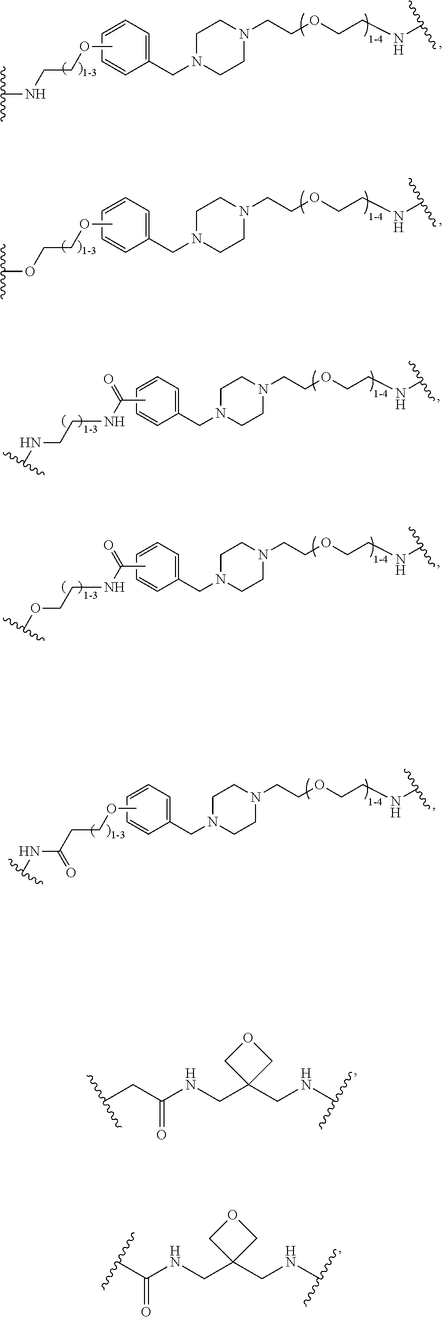

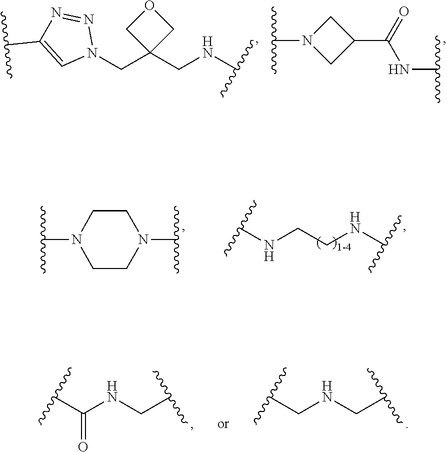

30. A compound of formula (III)

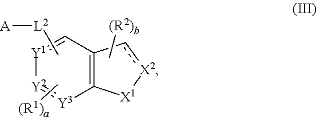

or a pharmaceutically acceptable salt, stereoisomer, or deuterated form thereof,
wherein:
   A is a ligand that binds to a protein, a protein aggregate, a protein complex, or a lipid;
   L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
   each R¹ is independently C₁₋₆ alkyl, C₃-C₈ cycloalkyl, or halogen;
   each R² is independently C₁₋₆ alkyl, C₃-C₈ cycloalkyl, or halogen, or two R² form an oxo;
   - - is a bond or absent;
   one of X¹ or X² is N-Q-R³ and the other is CH or CH₂ as permitted by valency, provided that when X² is N-Q-R³, - - is absent;
   Y¹, Y², and Y³ are each independently CH or N;
   Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
   R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
   each R⁴ is independently halogen, —OH, C₁₋₆ alkyl, C₁₋₆ alkoxy, or —C(O)O—(C₁₋₆ alkyl);
   R^{X} is H, C₁₋₆ alkyl, C₁₋₆ haloalkyl, or C₁₋₆ hydroxyalkyl;
   R^{Y} is C₃₋₈ cycloalkyl, C₃₋₈ hydroxycycloalkyl, aryl, or heterocycle;
   a is an integer of 0-3; and
   b is an integer of 0-2.

31. The compound of embodiment 30, or a pharmaceutically acceptable salt, a stereoisomer, or a deuterated form thereof, wherein the compound has a structure of formula (III-A):

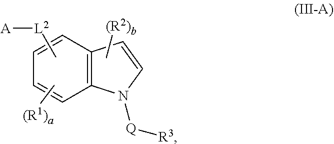

or a pharmaceutically acceptable salt, a stereoisomer, or a deuterated form thereof.

32. The compound of embodiment 30, or a pharmaceutically acceptable salt, a stereoisomer, or a deuterated form thereof, wherein the compound has a structure of formula (III-B), (III-C), or (III-D):

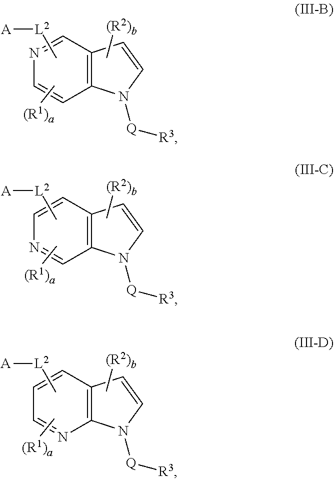

or a pharmaceutically acceptable salt, a stereoisomer, or a deuterated form thereof.

33. The compound of embodiment 30, or a pharmaceutically acceptable salt, a stereoisomer, or a deuterated form thereof, wherein the compound has a structure of formula (III-E), (III-F), or (III-G):

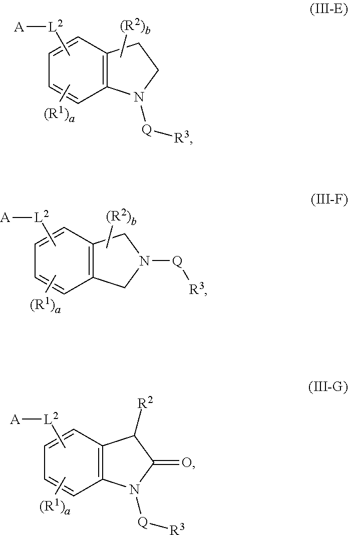

or a pharmaceutically acceptable salt, a stereoisomer, or a deuterated form thereof.

34. The compound of embodiment 31, or a pharmaceutically acceptable salt, a stereoisomer, or a deuterated form thereof, wherein the compound has a structure of formula (III-A-1), (III-A-2), or (III-A-3):

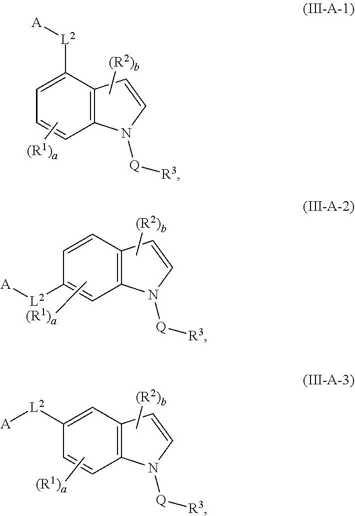

or a pharmaceutically acceptable salt, a stereoisomer, or a deuterated form thereof.

35. The compound of embodiment 32, or a pharmaceutically acceptable salt, a stereoisomer, or a deuterated form thereof, wherein the compound has a structure of formula (III-B-1), (III-C-1), or (III-D-1):

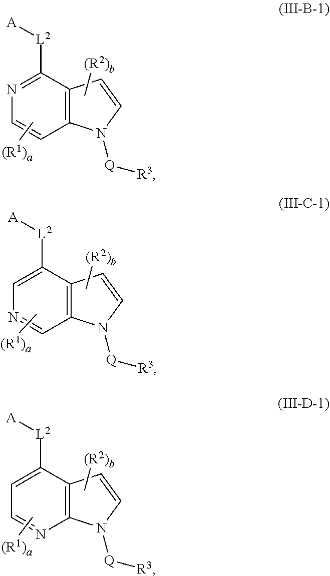

or a pharmaceutically acceptable salt, a stereoisomer, or a deuterated form thereof.

36. The compound of embodiment 33, or a pharmaceutically acceptable salt, a stereoisomer, or a deuterated form thereof, wherein the compound has a structure of formula (III-E-1), (III-F-1), or (III-G-1):

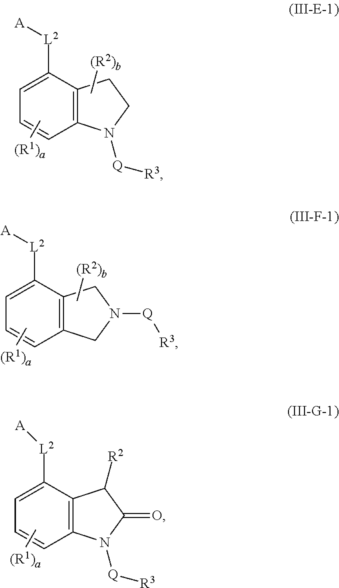

or a pharmaceutically acceptable salt, a stereoisomer, or a deuterated form thereof.

37. The compound of any one of embodiments 31-36, or a pharmaceutically acceptable salt, a stereoisomer, or a deuterated form thereof, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —N(H)C(O)—, —N(C₁-C₆ alkyl)C(O)—, —N(C₃-C₈ cycloalkyl)C(O)—, —C(O)N(H)—, —C(O)N(C₁-C₆ alkyl)-, —C(O)N(C₃-C₈ cycloalkyl)-, arylene, heteroarylene, heterocyclylene, or C₃-C₈ cycloalkylene, wherein each arylene or heteroarylene is optionally and independently substituted with 1 or 2 R^{Z}.

38. The compound of any one of embodiments 30-37, or a pharmaceutically acceptable salt, a stereoisomer, or a deuterated form thereof, wherein L² is:
—(C₁₋₆ alkylene)-NH(CH₂CH₂O)ₘ—(CH₂)ₙ—NH—,
—(C₁₋₆ alkylene)-NH(CH₂CH₂O)ₘ—(CH₂)ₙ—N(C₁₋₆ alkyl)-,
—(C₁₋₆ alkylene)-NH(CH₂CH₂O)ₘ—(CH₂)ₙ—,
—(C₁₋₆ alkylene)-NH(CH₂CH₂O)ₘ—(CH₂)ₙ—NH—C(O)—(C₁₋₆ alkylene)-O—, or
—(C₁₋₆ alkylene)-NH(CH₂CH₂O)ₘ—(CH₂)—HET^{B}-,
wherein HET^{B} is a 4-10 membered heterocyclylene containing 1-3 N, and is optionally substituted with —C(O)—(C₁₋₆ alkyl), —C(O)O—(C₁₋₆ alkyl), —(C₁₋₆ alkyl)-O—C₆₋₁₀ aryl, or C₁₋₆ alkyl;
m is an integer of 1-12; and
n is an integer of 0-6.

39. The compound of any one of embodiments 30-38, or a pharmaceutically acceptable salt, a stereoisomer, or a deuterated form thereof, wherein L² is:
—(CH₂)—NH(CH₂CH₂O)—(CH₂)₂—NH—,
—(CH₂)—NH(CH₂CH₂O)₂—(CH₂)₂—NH—,
—(CH₂)—NH(CH₂CH₂O)₃—(CH₂)₂—NH—,
—(CH₂)—NH(CH₂CH₂O)₃—(CH₂)₃—NCH₃—,
—(CH₂)—NH(CH₂CH₂O)₄—(CH₂)₃—NCH₃—,
—(CH₂)—NH(CH₂CH₂O)₃—,
—(CH₂)—NH(CH₂CH₂O)₄—,
—(CH₂)—NH(CH₂CH₂O)₃—(CH₂)₂—NH—C(O)CH₂—O—,
—(CH₂)—NH(CH₂CH₂O)₃—(CH₂)₂—,
—(CH₂)—NH(CH₂CH₂O)₃—(CH₂)₃—,

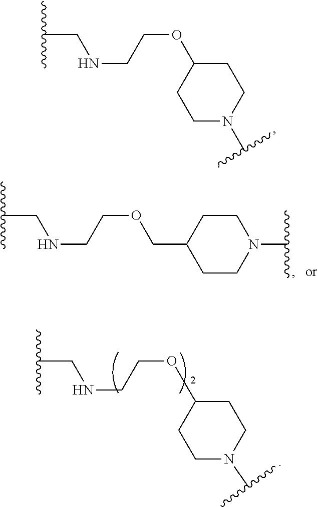

40. The compound of any one of embodiments 30-37, or a pharmaceutically acceptable salt, a stereoisomer, or a deuterated form thereof, wherein L² is:

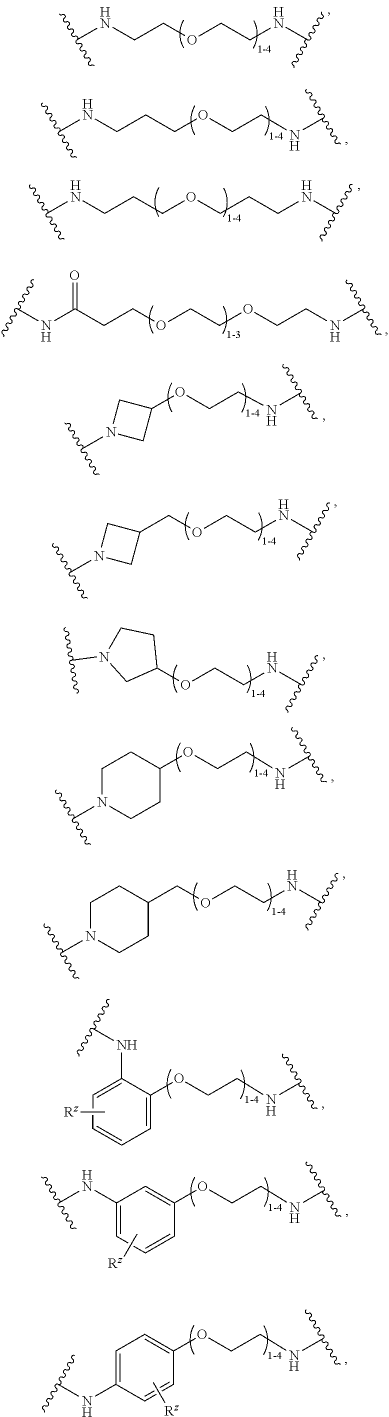

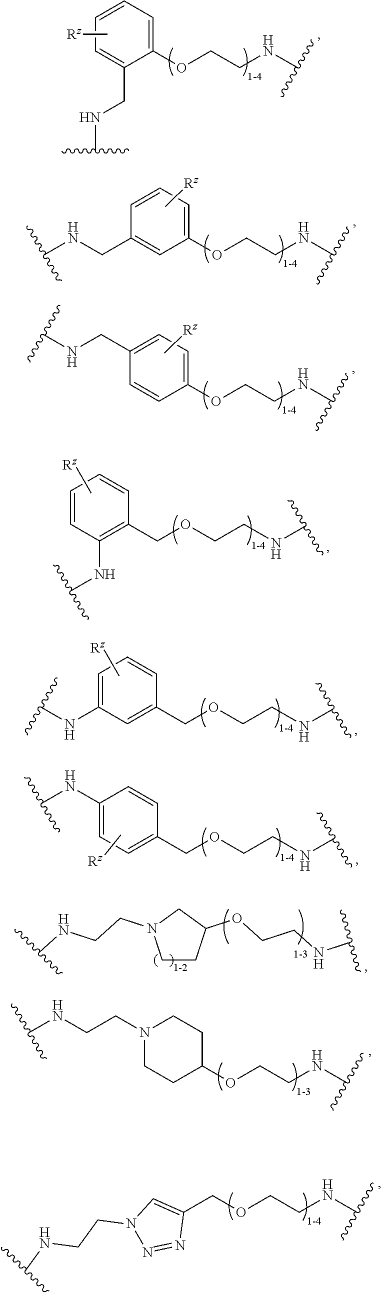

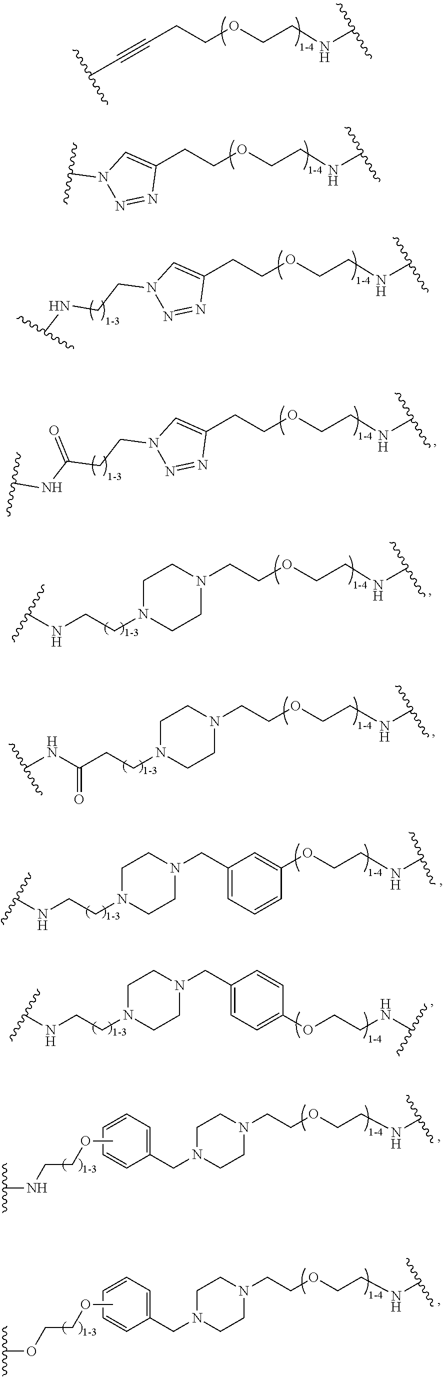

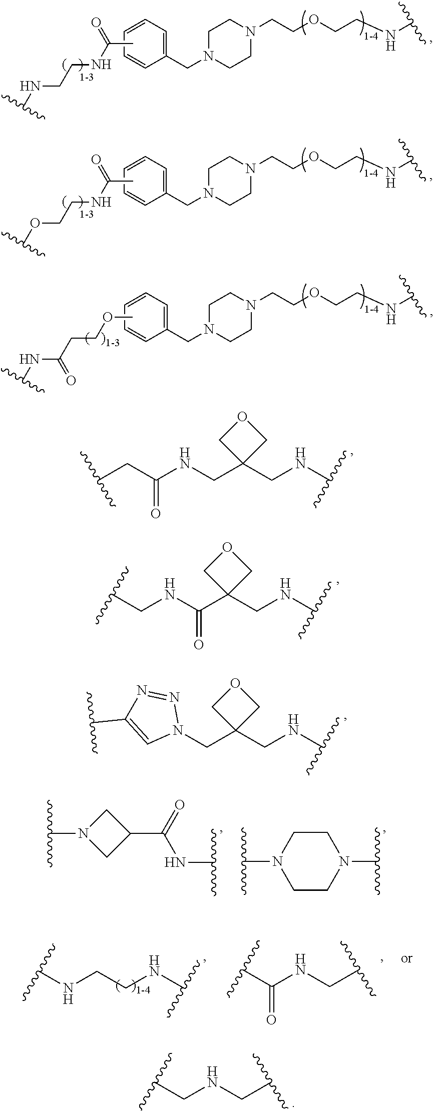

41. The compound of any one of embodiment 30-40, or a pharmaceutically acceptable salt, a stereoisomer, or a deuterated form thereof, wherein the protein is a protein that is associated with cancer.

42. The compound of embodiment 41, or a pharmaceutically acceptable salt, a stereoisomer, or a deuterated form thereof, wherein the protein associated with cancer comprises a mutation or a fusion.

43. The compound of embodiment 41 or 42, or a pharmaceutically acceptable salt, a stereoisomer, or a deuterated form thereof, wherein the protein associated with cancer is BRD4.

44. The compound of any one of embodiments 30-40, or a pharmaceutically acceptable salt, a stereoisomer, or a deuterated form thereof, wherein the protein is a protein associated with a metabolic disease.

45. The compound of any one of embodiments 30-40, or a pharmaceutically acceptable salt, a stereoisomer, or a deuterated form thereof, wherein the protein is a protein associated with inflammation.

46. The compound of any one of embodiments 30-40, or a pharmaceutically acceptable salt, a stereoisomer, or a deuterated form thereof, wherein the protein is present in bacteria.

47. The compound of any one of embodiments 30-40, or a pharmaceutically acceptable salt, a stereoisomer, or a deuterated form thereof, wherein the protein is present in a virus particle.

48. The compound of any one of embodiments 30-40, or a pharmaceutically acceptable salt, a stereoisomer, or a deuterated form thereof, wherein the protein aggregate is an alpha-synuclein protein aggregate, a mutant Huntingtin protein aggregate, an amyloid protein aggregate, or a TDP-43 protein aggregate.

49. The compound of any one of embodiments 30-40, or a pharmaceutically acceptable salt, a stereoisomer, or a deuterated form thereof, wherein the protein is an intracellular protein.

50. The compound of any one of embodiments 30-40, wherein A is aryl, arylene-heteroaryl, arylene, heteroarylene, aryl, heteroarylene-aryl, heteroarylene-heteroaryl, heteroarylene-heteroarylene-heteroaryl, heterocyclylene-aryl, heterocyclylene-heteroaryl, heteroarylene-NC(O)-heteroaryl, heteroarylene-N═N-aryl, or arylene-(arylalkyl)-OC(O)-hetercyclyl-C(O)C₁₋₆ alkylene-aryl, each of which is optionally substituted with 1, 2, 3, or 4 groups independently selected from halogen, C₁₋₆ alkyl, O—C₁₋₆ alkyl, NH₂, NH(C₁₋₆ alkyl), N(C₁₋₆ alkyl)₂, —(CH₂)₁₋₄C(O)NH₂, —(CH₂)₁₋₄C(O)NH(C₁₋₆ alkyl), or —(CH₂)₁₋₄C(O)N(C₁₋₆ alkyl)₂.

51. The compound of any one of embodiments 30-40 and 50, wherein A is

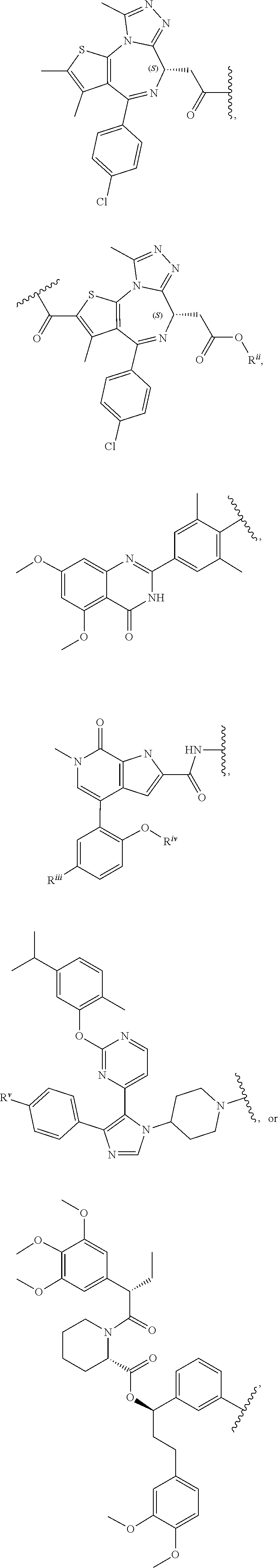

wherein:
Rⁱⁱ is C₁₋₆ alkyl;
Rⁱⁱⁱ is H,

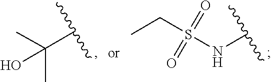

R^{iv} is

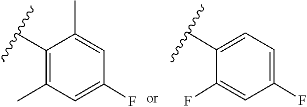

and
R^{v} is H, F, CF₃, or C₁₋₆ alkyl.

52. The compound of any one of embodiments 1-51, or a pharmaceutically acceptable salt, a stereoisomer, or a deuterated form thereof, wherein each R¹ is independent halogen.

53. The compound of embodiment 52, or a pharmaceutically acceptable salt, a stereoisomer, or a deuterated form thereof, wherein each R¹ is —F or —Cl.

54. The compound of any one of embodiments 1-53, or a pharmaceutically acceptable salt, a stereoisomer, or a deuterated form thereof, wherein a is 0 or 1.

55. The compound of any one of embodiments 1-54, or a pharmaceutically acceptable salt, a stereoisomer, or a deuterated form thereof, wherein each R² is independently C₁₋₆ alkyl.

56. The compound of embodiment 55, or a pharmaceutically acceptable salt, a stereoisomer, or a deuterated form thereof, wherein each R² is independently C₁₋₃ alkyl.

57. The compound of embodiment 55 or 56, or a pharmaceutically acceptable salt, a stereoisomer, or a deuterated form thereof, wherein each R² is methyl.

58. The compound of any one of embodiments 1-57, or a pharmaceutically acceptable salt, a stereoisomer, or a deuterated form thereof, wherein b is 0 or 1.

59. The compound of any one of embodiments 1-58, or a pharmaceutically acceptable salt, a stereoisomer, or a deuterated form thereof, wherein Q is a absent, C₁₋₄ alkylene, —(C₁₋₃ alkylene)C(O)—, C₃₋₆ cycloalkylene, —(C₃₋₆ cycloalkylene)C(O)—, —C(O)—, or —S(O)₂—.

60. The compound of embodiment 59, or a pharmaceutically acceptable salt, a stereoisomer, or a deuterated form thereof, wherein Q is a absent.

61. The compound of embodiment 59, or a pharmaceutically acceptable salt, a stereoisomer, or a deuterated form thereof, wherein Q is —CH₂—, —CH₂CH₂—, —CH(CH₃)—, or —C(CH₃)₂—.

62. The compound of embodiment 59, or a pharmaceutically acceptable salt, a stereoisomer, or a deuterated form thereof, wherein Q is —CH₂—.

63. The compound of embodiment 59, or a pharmaceutically acceptable salt, a stereoisomer, or a deuterated form thereof, wherein Q is —C(O)—.

64. The compound of embodiment 59, or a pharmaceutically acceptable salt, a stereoisomer, or a deuterated form thereof, wherein Q is —S(O)₂—.

65. The compound of embodiment 59, or a pharmaceutically acceptable salt, a stereoisomer, or a deuterated form thereof, wherein Q is.

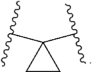 [SMILES: *C1(*)CC1]

66. The compound of embodiment 59, or a pharmaceutically acceptable salt, a stereoisomer, or a deuterated form thereof, wherein Q is —CH₂C(O)—.

67. The compound of embodiment 59, or a pharmaceutically acceptable salt, a stereoisomer, or a deuterated form thereof, wherein Q is

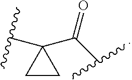 [SMILES: *C(=O)C1(*)CC1]

68. The compound of any one of embodiments 1-67, or a pharmaceutically acceptable salt, a stereoisomer, or a deuterated form thereof, wherein R³ is C₃₋₈ carbocycle, 3-10 membered heterocycle, C₆₋₁₀ aryl, or 5-10 membered heteroaryl, and wherein the C₃₋₈ carbocycle, 3-8 membered heterocycle, C₆₋₁₀ aryl, and 5-10 membered heteroaryl are each optionally substituted with 1 or 2 R⁴, and each R⁴ is independently halogen, —OH, C₁₋₆ alkyl, C₁₋₆ alkoxy, or —C(O)O—C₁₋₆ alkyl.

69. The compound of embodiment 68, or a pharmaceutically acceptable salt, a stereoisomer, or a deuterated form thereof, wherein R³ is C₆₋₁₀ aryl optionally substituted with 1 or 2 R⁴, and wherein each R⁴ is independently halogen, C₁₋₆ alkyl, or C₁₋₆ alkoxy.

70. The compound of embodiment 69, or a pharmaceutically acceptable salt, a stereoisomer, or a deuterated form thereof, wherein R³ is C₆₋₁₀ aryl optionally substituted with —F, —Cl, or C₁₋₃ alkoxy.

71. The compound of embodiment 69 or 70, or a pharmaceutically acceptable salt, a stereoisomer, or a deuterated form thereof, wherein R³ is

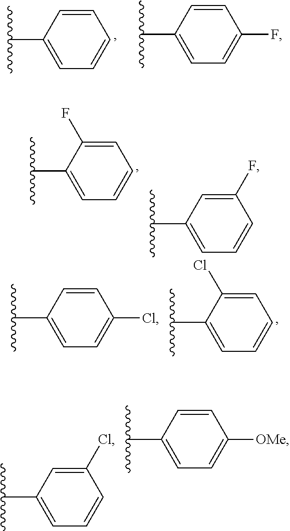

72. The compound of embodiment 68, or a pharmaceutically acceptable salt, a stereoisomer, or a deuterated form thereof, wherein R³ is 5-10 membered heteroaryl optionally substituted with 1 or 2 R⁴; and wherein the heteroaryl contains 1 or 2 heteroatoms selected from N, S, or O.

73. The compound of embodiment 72, or a pharmaceutically acceptable salt, a stereoisomer, or a deuterated form thereof, wherein R³ is

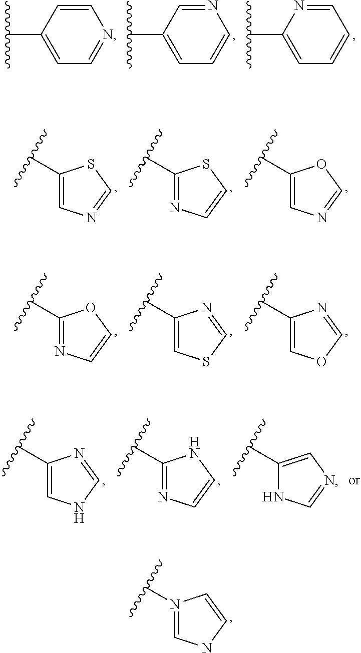

each optionally substituted with one R⁴.

74. The compound of embodiment 72 or 73, or a pharmaceutically acceptable salt, a stereoisomer, or a deuterated form thereof, wherein R³ is

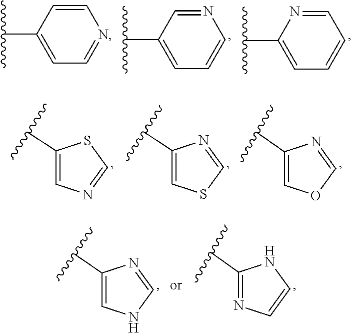

75. The compound of embodiment 68, or a pharmaceutically acceptable salt, a stereoisomer, or a deuterated form thereof, wherein R³ is C₃₋₈ carbocycle optionally substituted with 1 or 2 R⁴, and wherein each R⁴ is independently halogen, —OH, C₁₋₆ alkyl, or C₁₋₆ alkoxy.

76. The compound of embodiment 75, or a pharmaceutically acceptable salt, a stereoisomer, or a deuterated form thereof, wherein R³ is

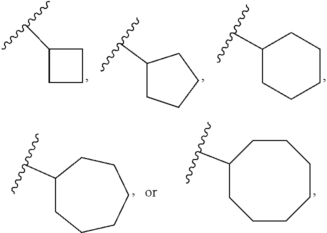

each optionally substituted with one R⁴.

77. The compound of embodiment 75 or 76, or a pharmaceutically acceptable salt, a stereoisomer, or a deuterated form thereof, wherein R³ is

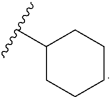 [SMILES: *C1CCCCC1]

78. The compound of embodiment 68, or a pharmaceutically acceptable salt, a stereoisomer, or a deuterated form thereof, wherein R³ is 3-10 membered heterocycle containing 1 or 2 heteroatoms selected from N, O, or S, and the heterocycle is optionally substituted with 1 or 2 R⁴, and wherein each R⁴ is independently —OH, C₁₋₆ alkyl, or —C(O)O—(C₁₋₆ alkyl).

79. The compound of embodiment 78, or a pharmaceutically acceptable salt, a stereoisomer, or a deuterated form thereof, wherein each R⁴ is independently —OH, C₁₋₃ alkyl, or —C(O)O—(C₁₋₅ alkyl).

80. The compound of embodiment 78 or 79, or a pharmaceutically acceptable salt, a stereoisomer, or a deuterated form thereof, wherein R³ is

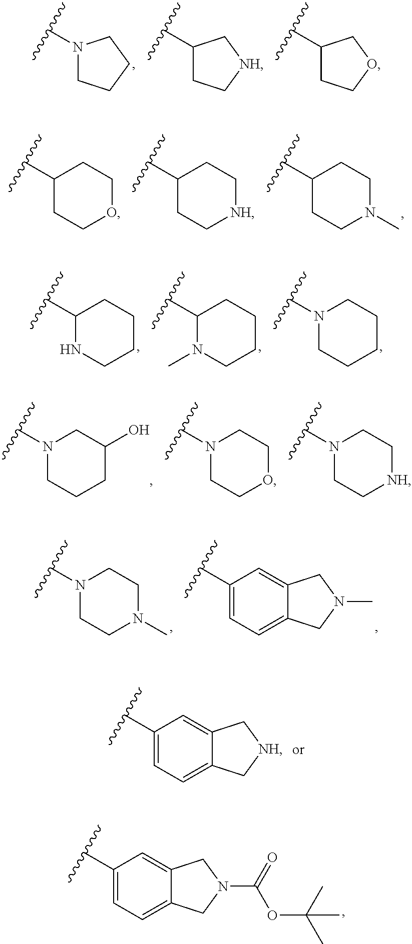

each optionally substituted with 1 or 2 R⁴.

81. The compound of any one of embodiments 78-80, or a pharmaceutically acceptable salt, a stereoisomer, or a deuterated form thereof, wherein

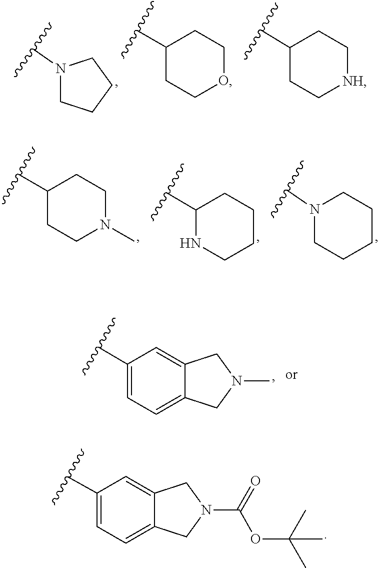

82. The compound of any one of embodiments 1-68, or a pharmaceutically acceptable salt, a stereoisomer, or a deuterated form thereof, wherein -Q-R³ is

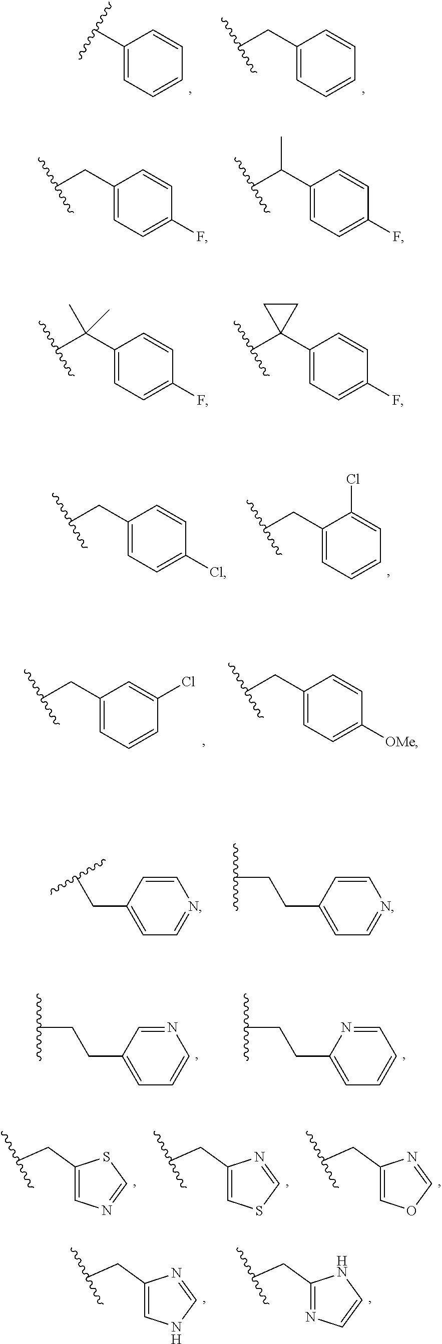

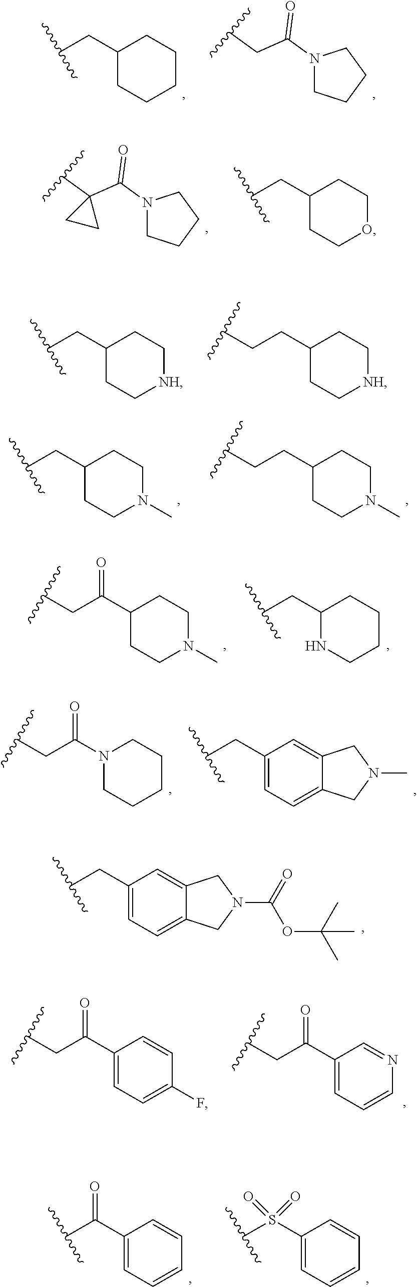

or —CH₂CH₂—NH—CH(CH₃)₂.

83. The compound of any one of embodiments 1-68 and 82, or a pharmaceutically acceptable salt, a stereoisomer, or a deuterated form thereof, wherein -Q-R³ is

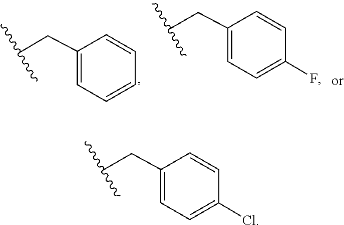

84. The compound of any one of embodiments 1, 2, and 5, wherein the compound of formula (I) is:

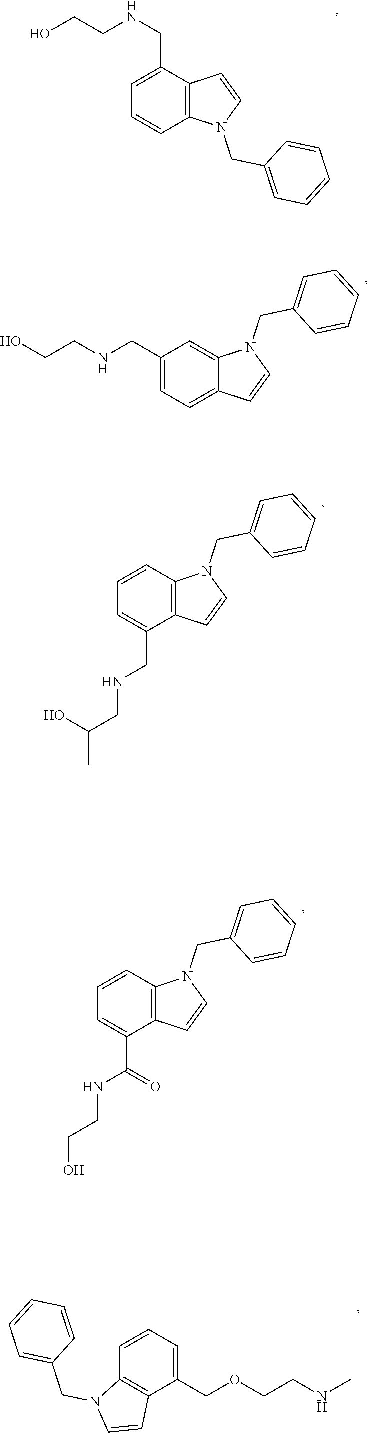 [SMILES: OCCNCc1cccc2c1ccn2Cc1ccccc1 ; OCCNCc1ccc2ccn(Cc3ccccc3)c2c1 ; CC(O)CNCc1cccc2c1ccn2Cc1ccccc1 ; O=C(NCCO)c1cccc2c1ccn2Cc1ccccc1 ; CNCCOCc1cccc2c1ccn2Cc1ccccc1]

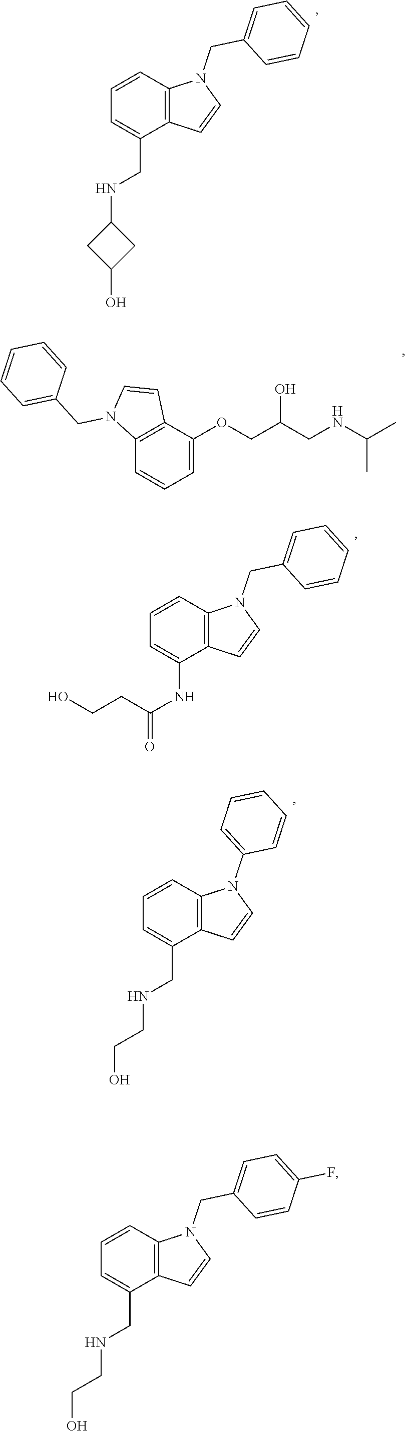 [SMILES: OC1CC(NCc2cccc3c2ccn3Cc2ccccc2)C1 ; CC(C)NCC(O)COc1cccc2c1ccn2Cc1ccccc1 ; O=C(CCO)Nc1cccc2c1ccn2Cc1ccccc1 ; OCCNCc1cccc2c1ccn2-c1ccccc1 ; OCCNCc1cccc2c1ccn2Cc1ccc(F)cc1]

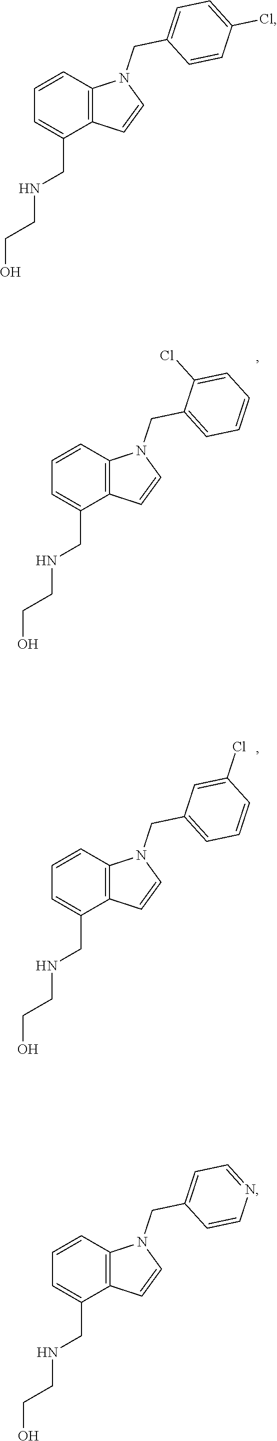 [SMILES: OCCNCc1cccc2c1ccn2Cc1ccc(Cl)cc1 ; OCCNCc1cccc2c1ccn2Cc1ccccc1Cl ; OCCNCc1cccc2c1ccn2Cc1cccc(Cl)c1 ; OCCNCc1cccc2c1ccn2Cc1ccncc1]

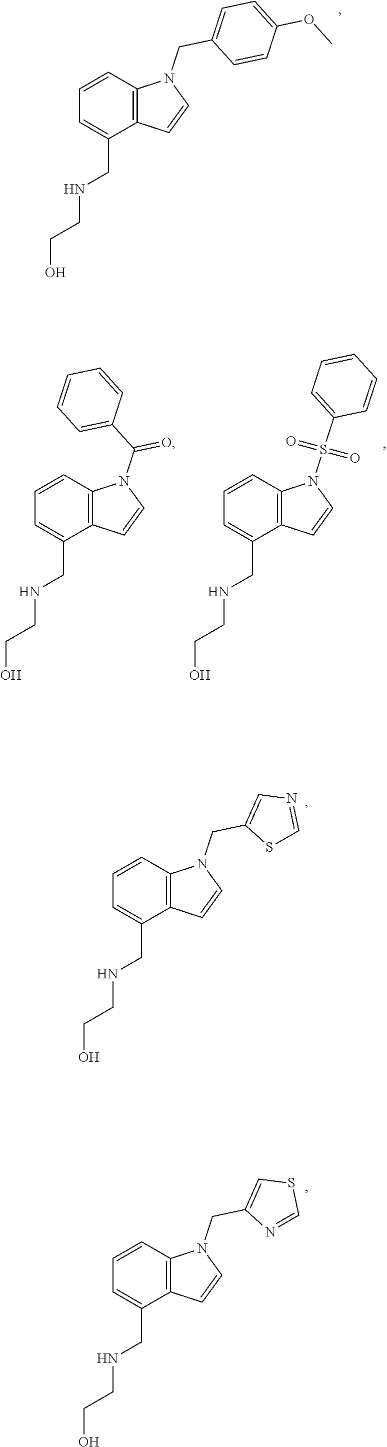 [SMILES: COc1ccc(Cn2ccc3c(CNCCO)cccc32)cc1 ; O=C(c1ccccc1)n1ccc2c(CNCCO)cccc21 ; O=S(=O)(c1ccccc1)n1ccc2c(CNCCO)cccc21 ; OCCNCc1cccc2c1ccn2Cc1cncs1 ; OCCNCc1cccc2c1ccn2Cc1cscn1]

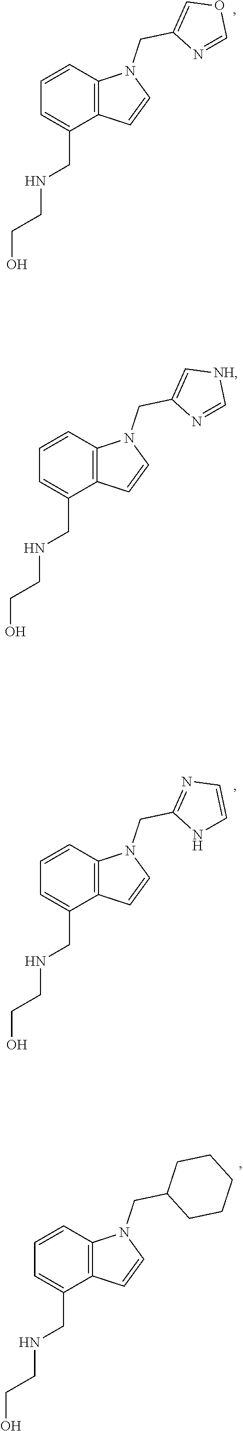 [SMILES: OCCNCc1cccc2c1ccn2Cc1cocn1 ; OCCNCc1cccc2c1ccn2Cc1c[nH]cn1 ; OCCNCc1cccc2c1ccn2Cc1ncc[nH]1 ; OCCNCc1cccc2c1ccn2CC1CCCCC1]

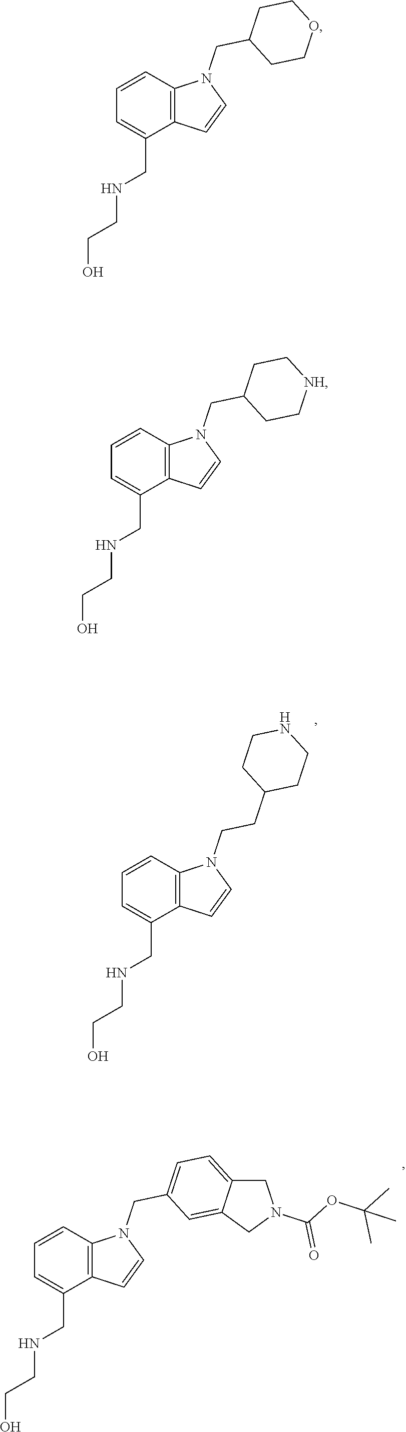 [SMILES: OCCNCc1cccc2c1ccn2CC1CCOCC1 ; OCCNCc1cccc2c1ccn2CC1CCNCC1 ; OCCNCc1cccc2c1ccn2CCC1CCNCC1 ; CC(C)(C)OC(=O)N1Cc2ccc(Cn3ccc4c(CNCCO)cccc43)cc2C1]

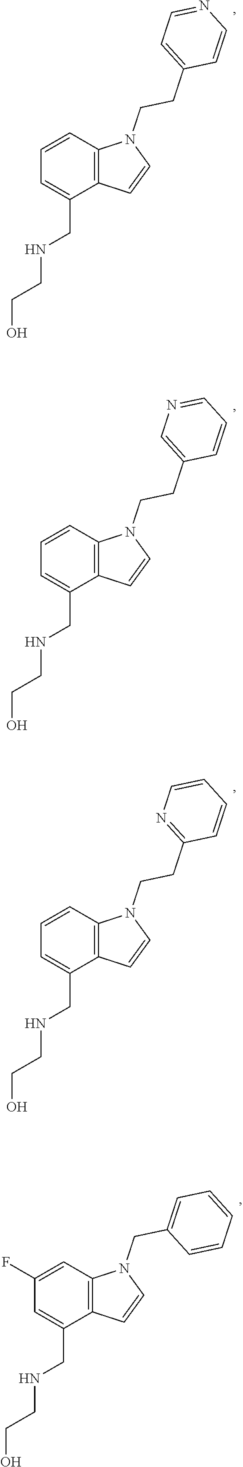 [SMILES: OCCNCc1cccc2c1ccn2CCc1ccncc1 ; OCCNCc1cccc2c1ccn2CCc1cccnc1 ; OCCNCc1cccc2c1ccn2CCc1ccccn1 ; OCCNCc1cc(F)cc2c1ccn2Cc1ccccc1]

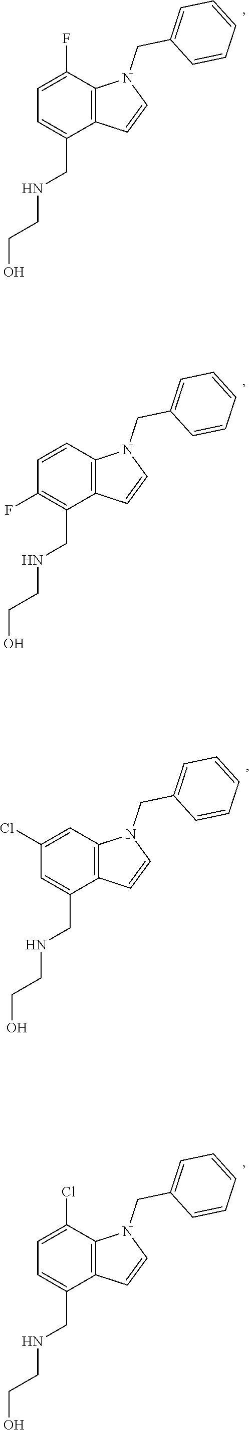 [SMILES: OCCNCc1ccc(F)c2c1ccn2Cc1ccccc1 ; OCCNCc1c(F)ccc2c1ccn2Cc1ccccc1 ; OCCNCc1cc(Cl)cc2c1ccn2Cc1ccccc1 ; OCCNCc1ccc(Cl)c2c1ccn2Cc1ccccc1]

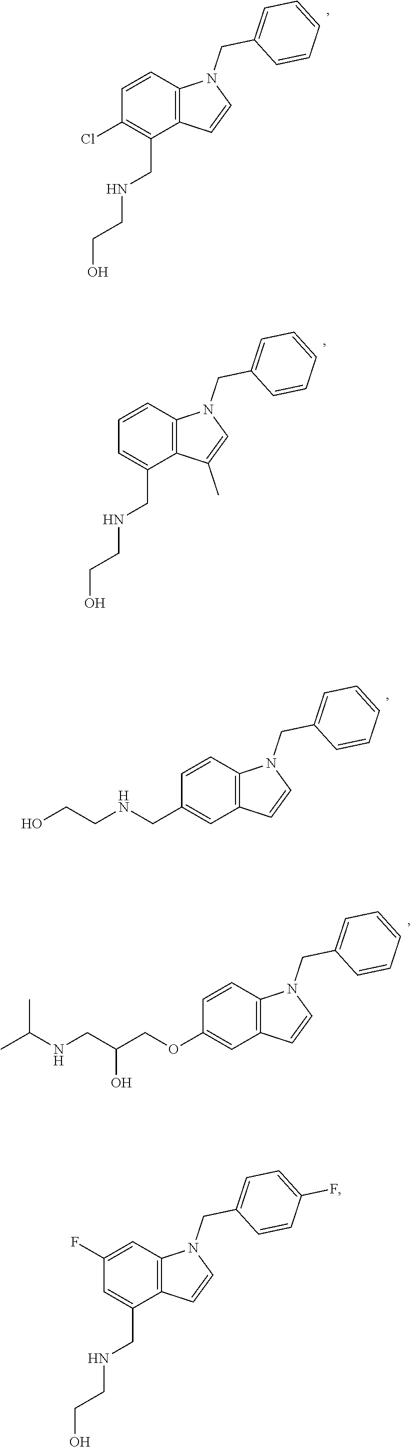 [SMILES: OCCNCc1c(Cl)ccc2c1ccn2Cc1ccccc1 ; Cc1cn(Cc2ccccc2)c2cccc(CNCCO)c12 ; OCCNCc1ccc2c(ccn2Cc2ccccc2)c1 ; CC(C)NCC(O)COc1ccc2c(ccn2Cc2ccccc2)c1 ; OCCNCc1cc(F)cc2c1ccn2Cc1ccc(F)cc1]

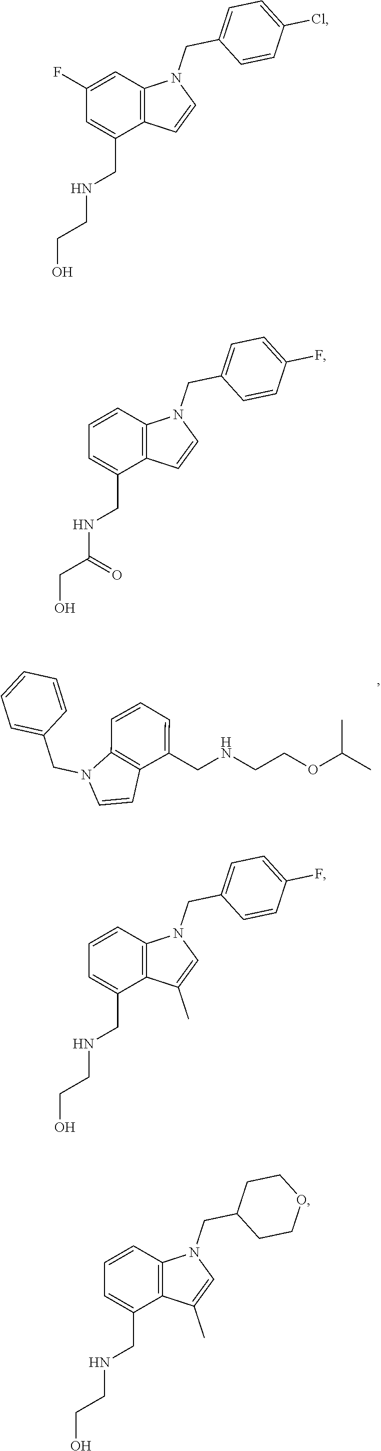 [SMILES: OCCNCc1cc(F)cc2c1ccn2Cc1ccc(Cl)cc1 ; O=C(CO)NCc1cccc2c1ccn2Cc1ccc(F)cc1 ; CC(C)OCCNCc1cccc2c1ccn2Cc1ccccc1 ; Cc1cn(Cc2ccc(F)cc2)c2cccc(CNCCO)c12 ; Cc1cn(CC2CCOCC2)c2cccc(CNCCO)c12]

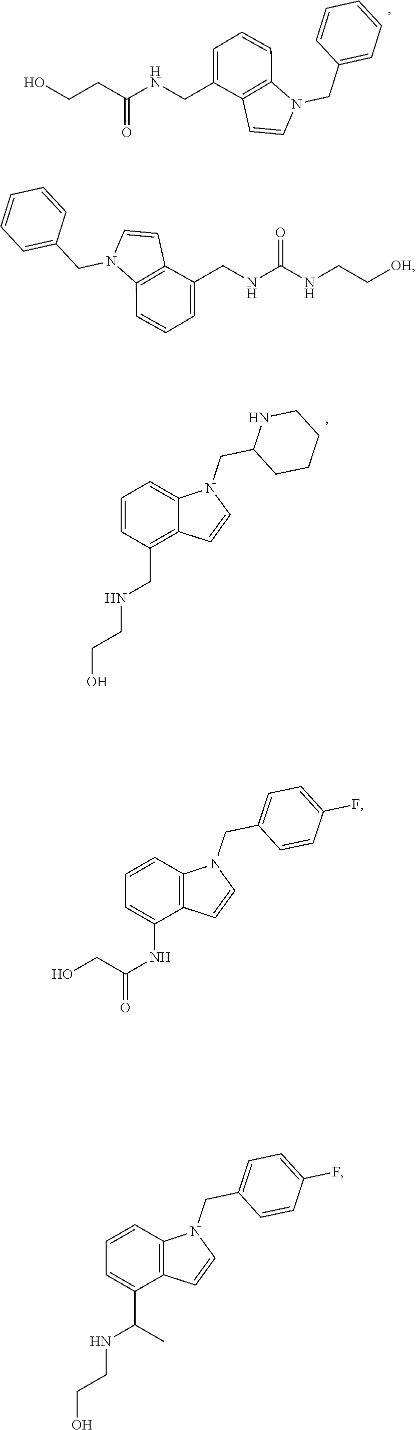 [SMILES: O=C(CCO)NCc1cccc2c1ccn2Cc1ccccc1 ; O=C(NCCO)NCc1cccc2c1ccn2Cc1ccccc1 ; OCCNCc1cccc2c1ccn2CC1CCCCN1 ; O=C(CO)Nc1cccc2c1ccn2Cc1ccc(F)cc1 ; CC(NCCO)c1cccc2c1ccn2Cc1ccc(F)cc1]

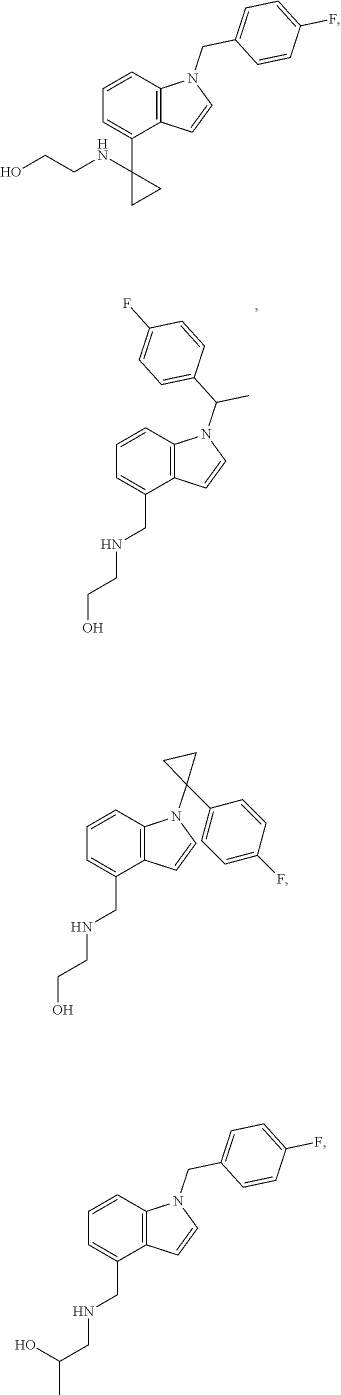 [SMILES: OCCNC1(c2cccc3c2ccn3Cc2ccc(F)cc2)CC1 ; CC(c1ccc(F)cc1)n1ccc2c(CNCCO)cccc21 ; OCCNCc1cccc2c1ccn2C1(c2ccc(F)cc2)CC1 ; CC(O)CNCc1cccc2c1ccn2Cc1ccc(F)cc1]

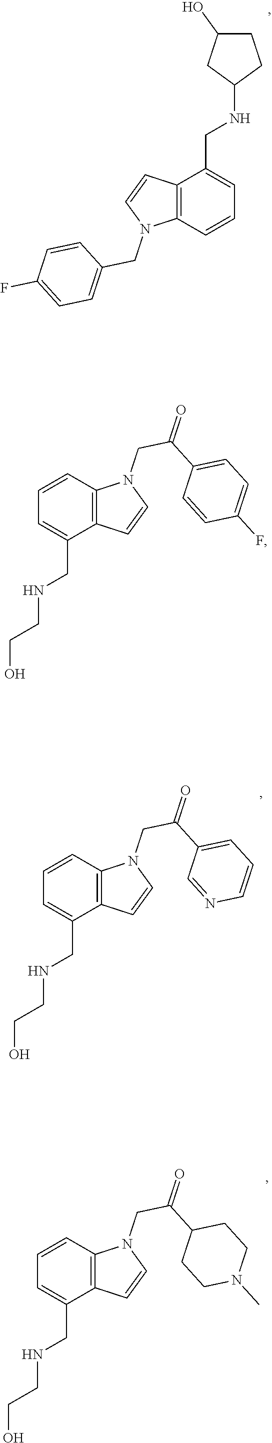 [SMILES: OC1CCC(NCc2cccc3c2ccn3Cc2ccc(F)cc2)C1 ; O=C(Cn1ccc2c(CNCCO)cccc21)c1ccc(F)cc1 ; O=C(Cn1ccc2c(CNCCO)cccc21)c1cccnc1 ; CN1CCC(C(=O)Cn2ccc3c(CNCCO)cccc32)CC1]

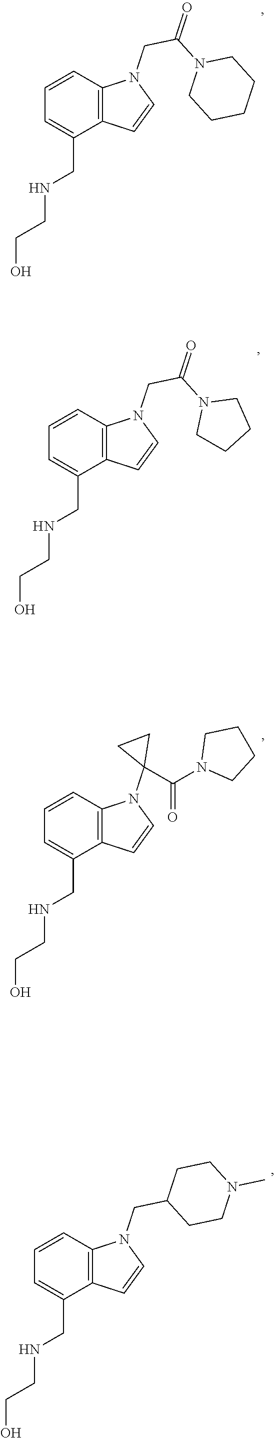 [SMILES: O=C(Cn1ccc2c(CNCCO)cccc21)N1CCCCC1 ; O=C(Cn1ccc2c(CNCCO)cccc21)N1CCCC1 ; O=C(N1CCCC1)C1(n2ccc3c(CNCCO)cccc32)CC1 ; CN1CCC(Cn2ccc3c(CNCCO)cccc32)CC1]

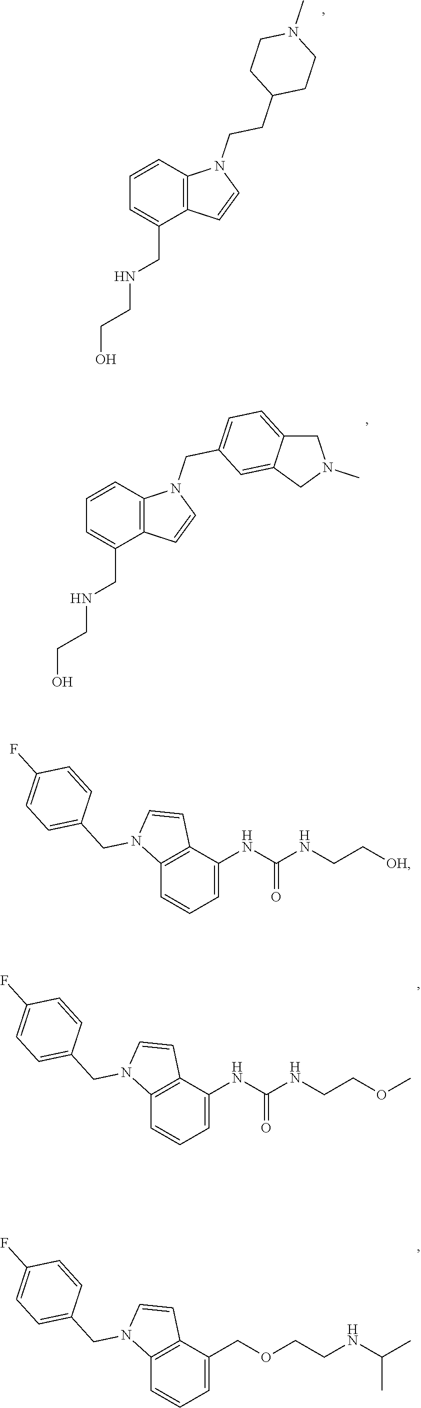 [SMILES: CN1CCC(CCn2ccc3c(CNCCO)cccc32)CC1 ; CN1Cc2ccc(Cn3ccc4c(CNCCO)cccc43)cc2C1 ; O=C(NCCO)Nc1cccc2c1ccn2Cc1ccc(F)cc1 ; COCCNC(=O)Nc1cccc2c1ccn2Cc1ccc(F)cc1 ; CC(C)NCCOCc1cccc2c1ccn2Cc1ccc(F)cc1]

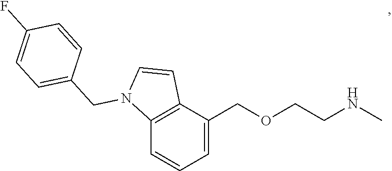 [SMILES: CNCCOCc1cccc2c1ccn2Cc1ccc(F)cc1]

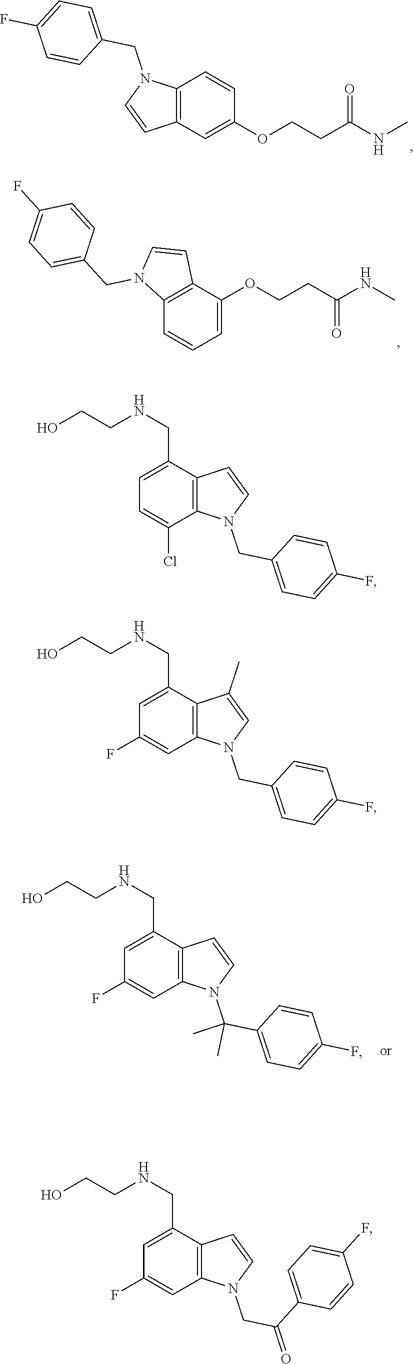 [SMILES: CNC(=O)CCOc1ccc2c(ccn2Cc2ccc(F)cc2)c1 ; CNC(=O)CCOc1cccc2c1ccn2Cc1ccc(F)cc1 ; OCCNCc1ccc(Cl)c2c1ccn2Cc1ccc(F)cc1 ; Cc1cn(Cc2ccc(F)cc2)c2cc(F)cc(CNCCO)c12 ; CC(C)(c1ccc(F)cc1)n1ccc2c(CNCCO)cc(F)cc21 ; O=C(Cn1ccc2c(CNCCO)cc(F)cc21)c1ccc(F)cc1]

or a pharmaceutically acceptable salt, a stereoisomer, or deuterated form thereof.

85. The compound of any one of embodiments 1, 3, and 6, wherein the compound of formula (I) is:

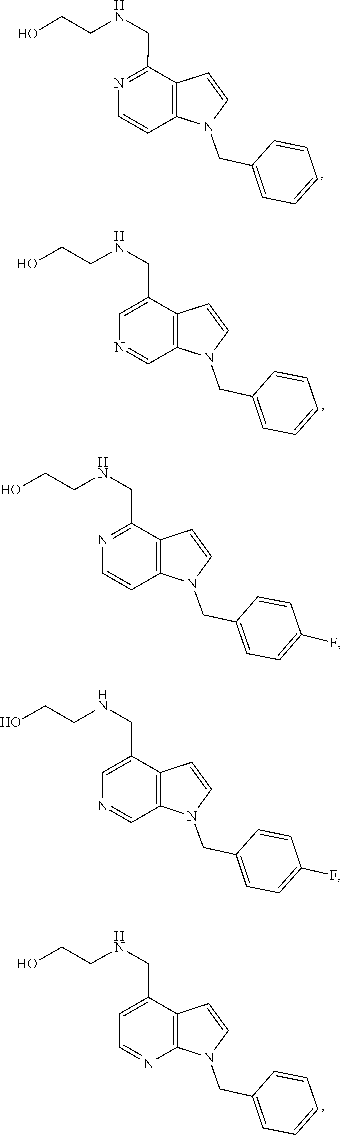 [SMILES: OCCNCc1nccc2c1ccn2Cc1ccccc1 ; OCCNCc1cncc2c1ccn2Cc1ccccc1 ; OCCNCc1nccc2c1ccn2Cc1ccc(F)cc1 ; OCCNCc1cncc2c1ccn2Cc1ccc(F)cc1 ; OCCNCc1ccnc2c1ccn2Cc1ccccc1]

or a pharmaceutically acceptable salt or deuterated form thereof.

86. The compound of any one of embodiments 1, 4, and 7, wherein the compound of formula (I) is:

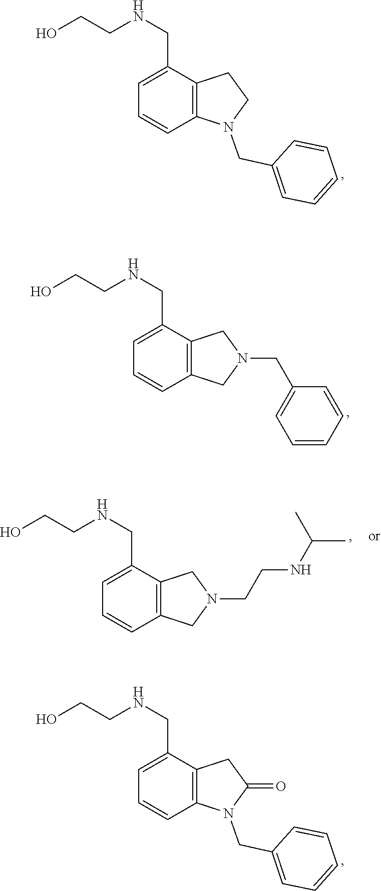 [SMILES: OCCNCc1cccc2c1CCN2Cc1ccccc1 ; OCCNCc1cccc2c1CN(Cc1ccccc1)C2 ; CC(C)NCCN1Cc2cccc(CNCCO)c2C1 ; O=C1Cc2c(CNCCO)cccc2N1Cc1ccccc1]

or a pharmaceutically acceptable salt or deuterated form thereof.

87. The compound of embodiment 15, wherein the compound of formula (II) is

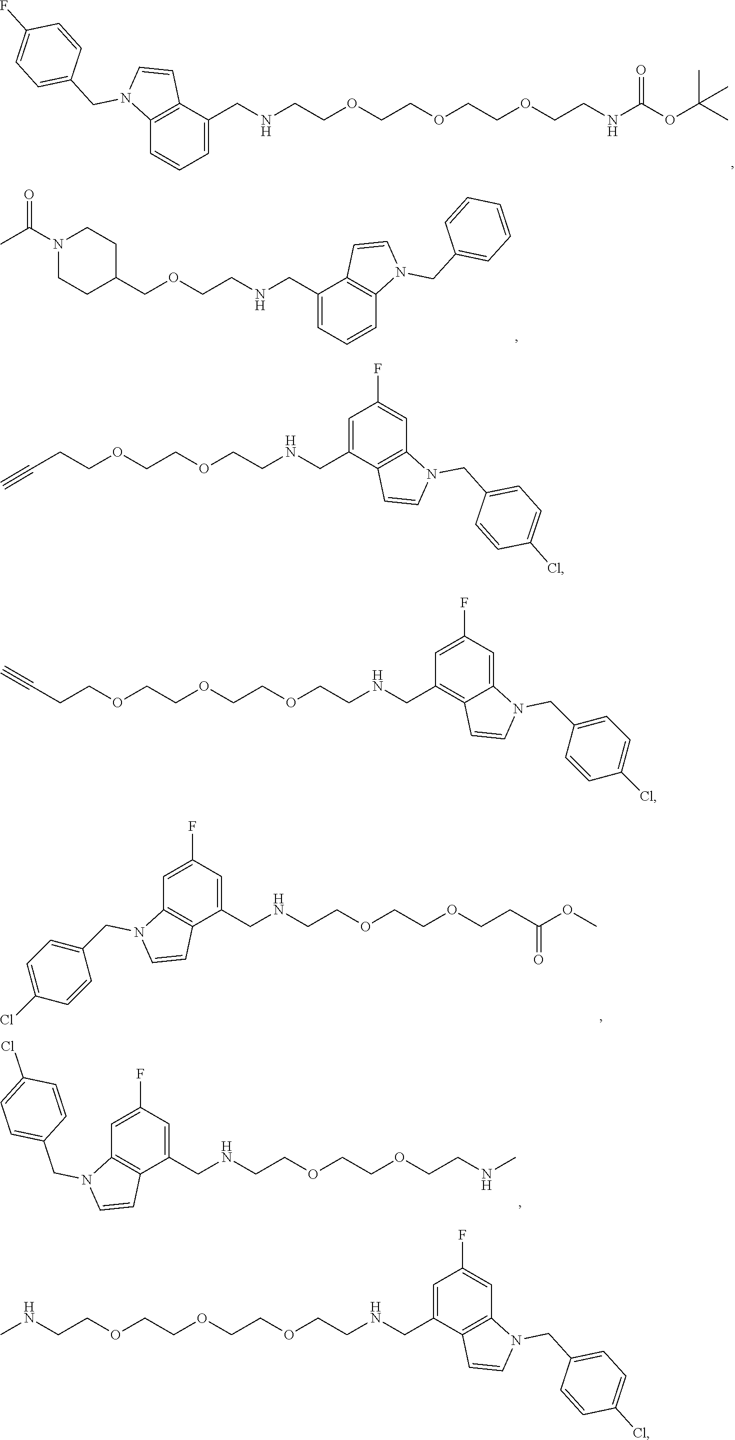

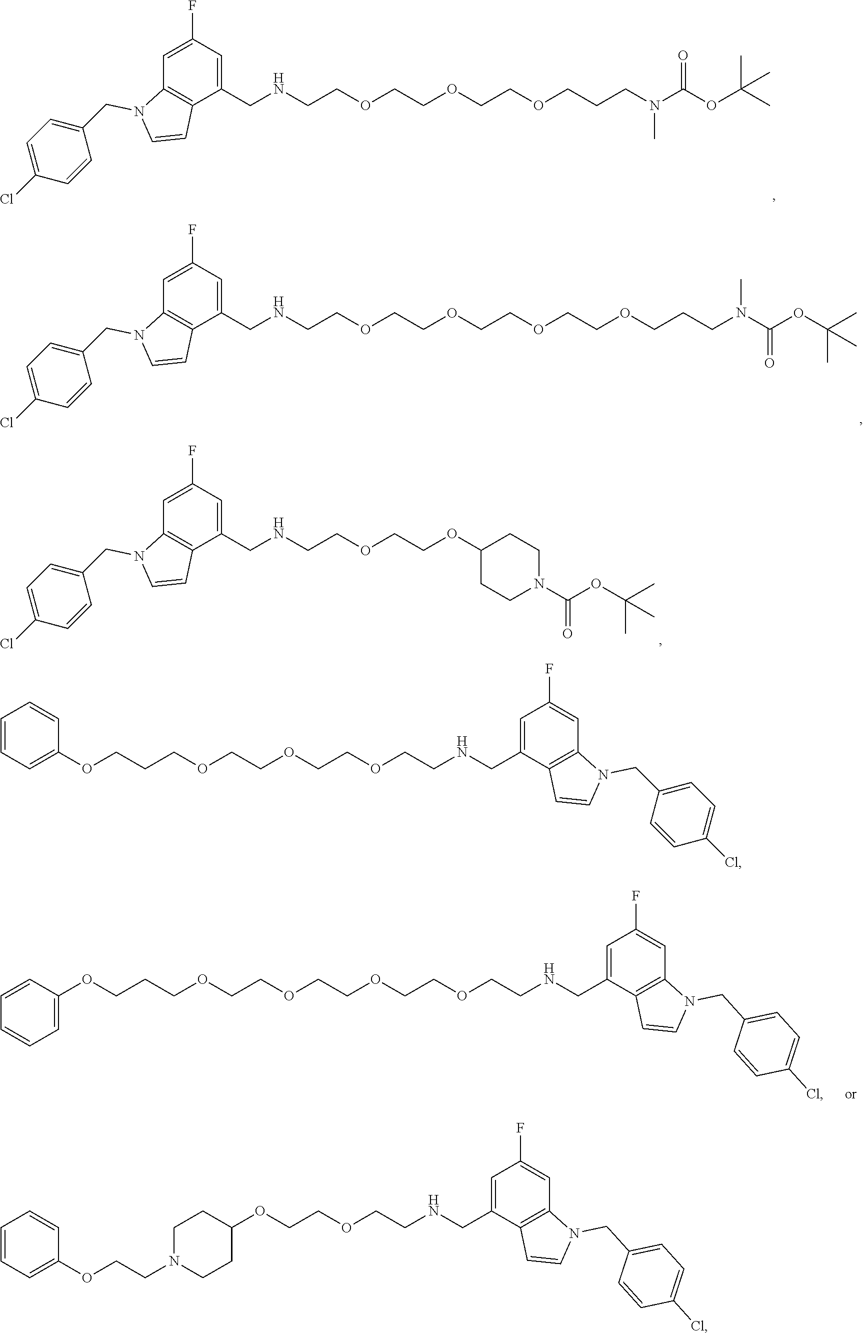

or a pharmaceutically acceptable salt, a stereoisomer, or deuterated form thereof.

88. The compound of embodiment 30, wherein the compound of formula (III) is

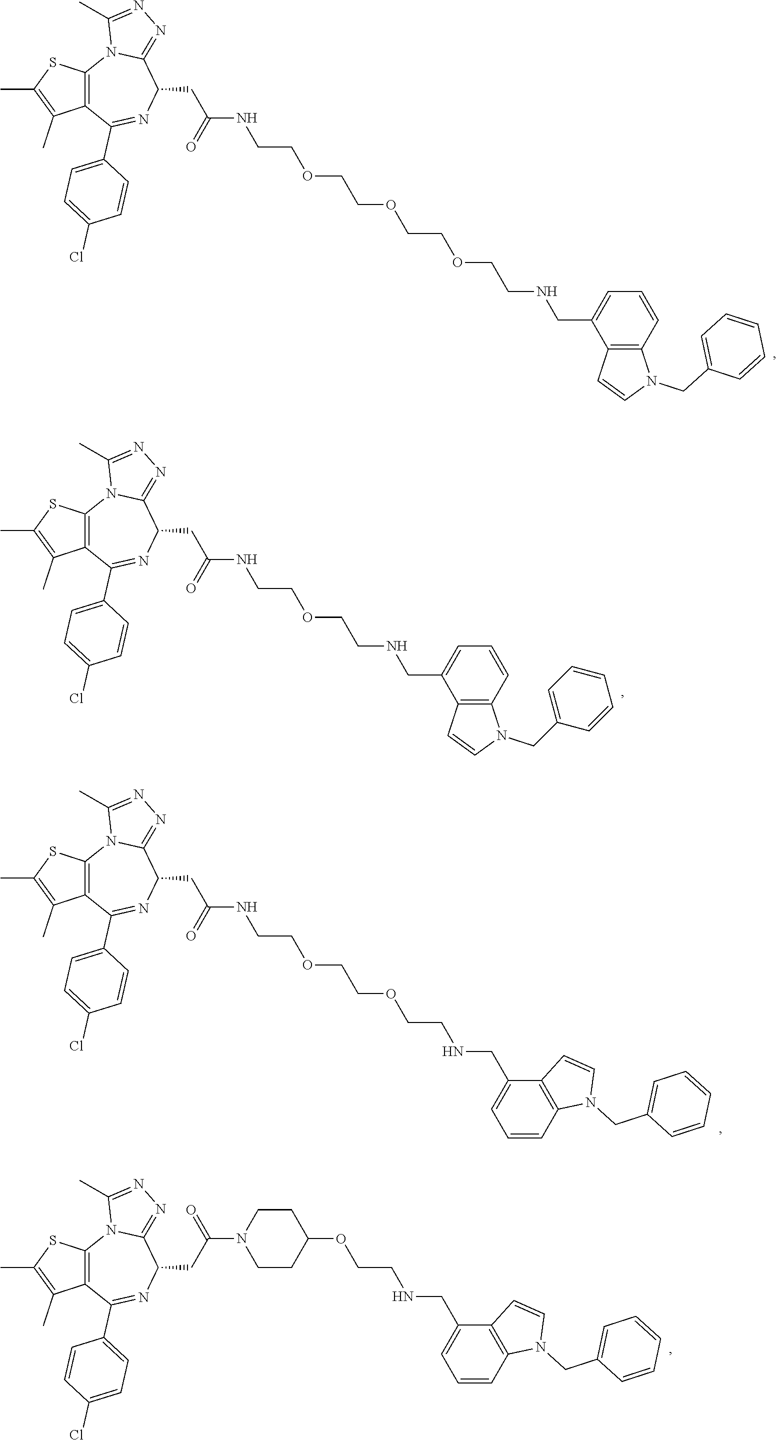 [SMILES: Cc1sc2c(c1C)C(c1ccc(Cl)cc1)=N[C@@H](CC(=O)NCCOCCOCCOCCNCc1cccc3c1ccn3Cc1ccccc1)c1nnc(C)n1-2 ; Cc1sc2c(c1C)C(c1ccc(Cl)cc1)=N[C@@H](CC(=O)NCCOCCNCc1cccc3c1ccn3Cc1ccccc1)c1nnc(C)n1-2 ; Cc1sc2c(c1C)C(c1ccc(Cl)cc1)=N[C@@H](CC(=O)NCCOCCOCCNCc1cccc3c1ccn3Cc1ccccc1)c1nnc(C)n1-2 ; Cc1sc2c(c1C)C(c1ccc(Cl)cc1)=N[C@@H](CC(=O)N1CCC(OCCNCc3cccc4c3ccn4Cc3ccccc3)CC1)c1nnc(C)n1-2]

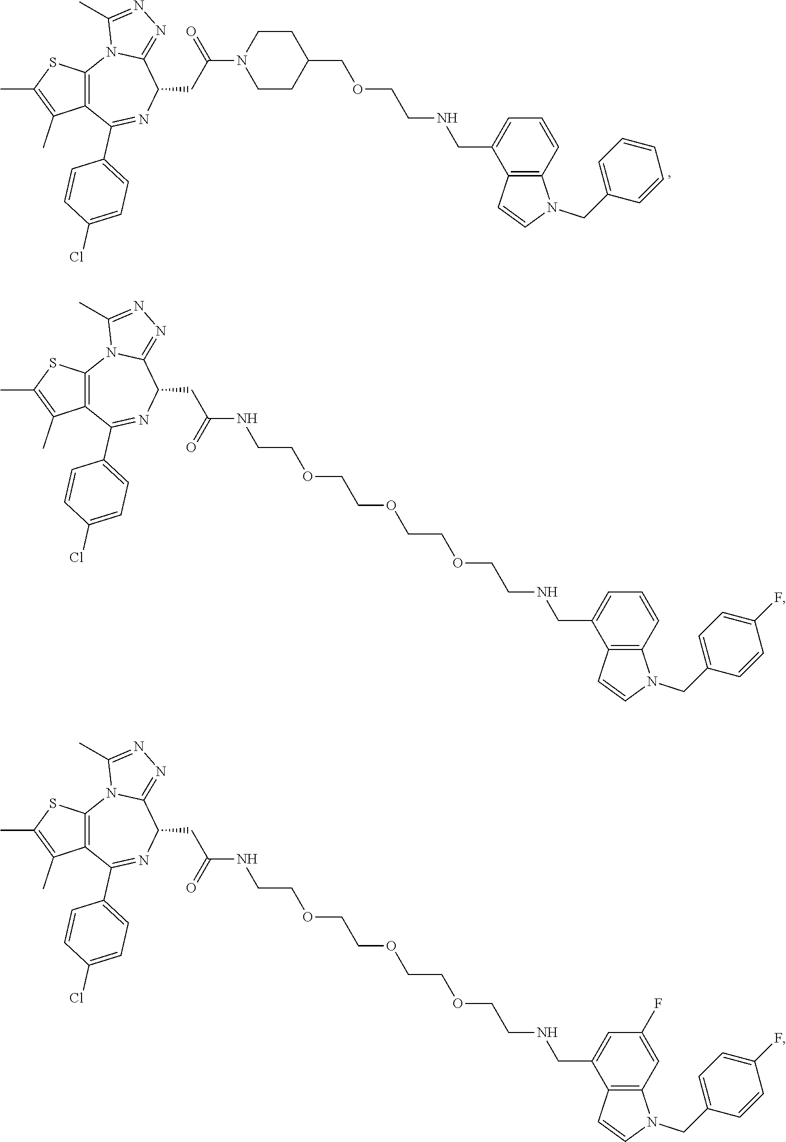

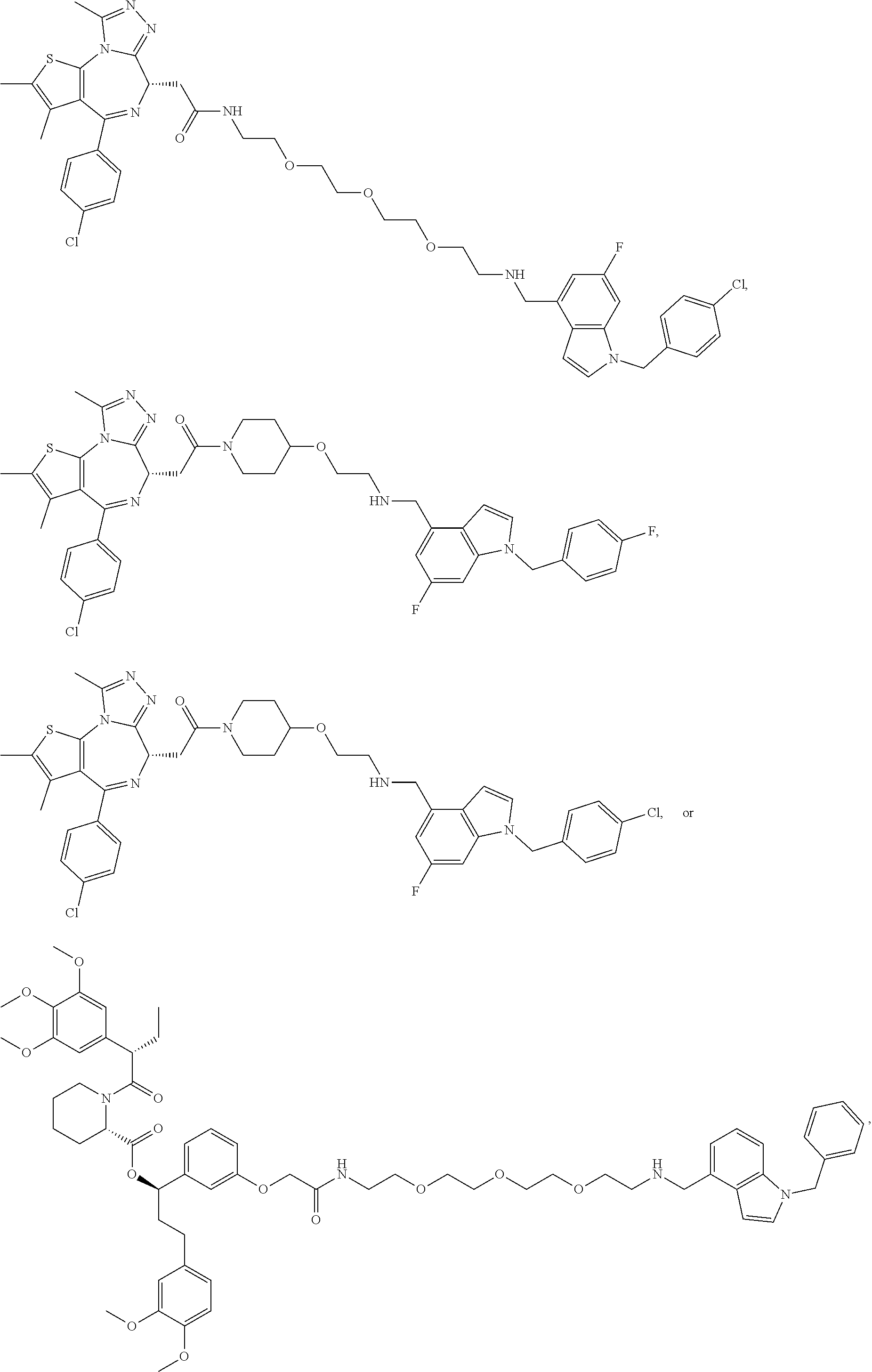

or a pharmaceutically acceptable salt, a stereoisomer, or deuterated form thereof.

89. A method of modulating autophagy in a subject, comprising administering to the subject a compound of any one of the preceding embodiments.

90. The method of embodiment 89, wherein the compound contacts p62.

91. The method of embodiment 90, wherein the compound increases activity of p62, thereby causing autophagy.

92. The method of embodiment 90, wherein is the compound decreases activity of p62, thereby reducing autophagy.

93. A method of degrading a target protein in a subject in need thereof, comprising administering a compound of any one of the preceding embodiments.

94. A method of inducing autophagy in a subject in need thereof, comprising administering a compound of any one of the preceding embodiments.

95. The method of embodiment 94, wherein the compound contacts p62, thereby inducing autophagy.

### EXAMPLES

The present disclosure is further illustrated by reference to the following Examples. However, it should be noted that these Examples, like the embodiments described above, are illustrative and are not to be construed as restricting the scope of the disclosure in any way.

In embodiments, compounds of the present disclosure can be synthesized using the following methods. General reaction conditions are given, and reaction products can be purified by generally known methods including silica gel chromatography using various organic solvents such as hexane, dichloromethane, ethyl acetate, methanol and the like or preparative reverse phase high pressure liquid chromatography.

### Abbreviations

equiv. equivalence
RT room temperature
TLC thin layer chromatography
ACN acetonitrile
AcOH acetic acid
EtOAc ethyl acetate
FA formic acid
Bz benzoyl
TBAS tetrabutylammonium hydrogen sulfate
min minutes
h hour
THE tetrahydrofuran
Pet. petroleum
aq. aqueous
br. broad
sat. saturated
DMEDA N,N′-Dimethylethylenediamine
DIBAL-H Diisobutylaluminium hydride
DMP Dess-Martin periodinane
DIPEA N,N′-Diisopropylethylamine
HATU Hexafluorophosphate Azabenzotriazole Tetramethyl Uronium
Fmoc-Osu N-(9-Fluorenylmethoxycarbonyloxy)succinimide
TFA trifluoroacetic acid
Cbz benzyl chloroformate
HOBt 1-Hydroxybenzotriazole
EDC·HCl (1-ethyl-3-(3-dimethylaminopropyl)carbodiimide hydrochloride)

### Example 1: Synthesis of Compound 1 Via Synthesis Route A

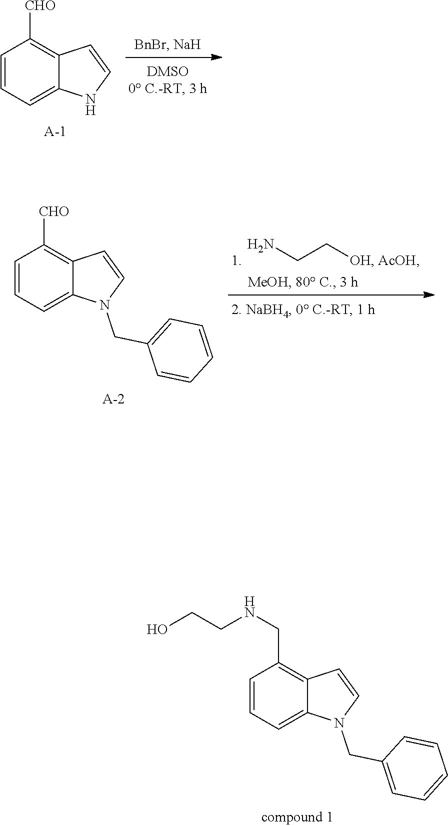

### Synthesis of 1-benzyl-1H-indole-4-carbaldehyde (A-2)

To a stirred solution of 1H-indole-4-carbaldehyde A-1 (500 mg, 3.44 mmol, 1.0 equiv.) in DMSO (10 mL) at 0° C., was added NaH (206 mg, 5.17 mmol, 1.5 equiv.) and stirred for 30 mins at the same temperature. Benzyl bromide (0.66 mL, 3.44 mmol, 1.0 equiv.) was then added and the reaction mixture was stirred at RT for 3 h. After completion of reaction by TLC, the reaction mixture was quenched with ice-cold water and extracted with ethyl acetate. The organic layer was washed with brine solution, filtered over anhydrous sodium sulphate, and concentrated under reduced pressure to afford crude material (1.2 g). The crude material was purified by silica gel flash chromatography (elution with 10% EtOAc in hexane) to afford A-2 as a pale-yellow gummy solid (700 mg, yield: 86%). TLC system: EtOAc/hexane (20:80), R_{f} value=0.6; ¹H NMR (400 MHz, CDCl₃) δ ppm: 10.26 (s, 1H), 7.63 (dd, J=7.2 Hz, 1.2 Hz, 1H), 7.55 (d, J=8.0 Hz, 1H), 7.36-7.34 (m, 2H), 7.30-7.27 (m, 4H), 7.10-7.08 (m, 2H), 5.40 (s, 2H).

### Synthesis of 2-(((1-benzyl-1H-indol-4-yl)methyl)amino)ethan-1-ol as formic acid salt (Compound 1)

To a stirred solution of A-2 (300 mg, 1.27 mmol, 1.0 equiv.) in MeOH (6 mL) at RT, was added 2-aminoethan-1-ol (195 mg, 3.17 mmol, 2.5 equiv.) and AcOH (0.1 mL, 1.91 mmol, 1.5 equiv.). The reaction mixture was stirred at 80° C. for 3 h before the mixture was cooled to 0° C. and NaBH₄ (96 mg, 2.55 mmol, 2 equiv.) was added portion-wise. The resultant mixture was stirred at RT for 1 h and upon completion of reaction by TLC, the reaction mixture was concentrated under reduced pressure and purified by reverse phase column chromatography (Grace) (elution with 20-25% ACN in 0.05% FA in H₂O) to afford compound 1 (formic acid salt) as an off-white solid (60 mg, yield: 20%). TLC system: MeOH/CH₂Cl₂ (10:90), R_{f} value=0.15; H NMR (400 MHz, DMSO-d₆) δ ppm: 8.25 (br. s, 1H; HCOOH), 7.50 (d, J=3.2 Hz, 1H), 7.36-7.34 (m, 1H), 7.31-7.27 (m, 2H), 7.25-7.22 (m, 1H), 7.21-7.18 (m, 2H), 7.08-7.03 (m, 2H), 6.62 (dd, J=3.2 Hz, 0.4 Hz, 1H), 5.42 (s, 2H), 4.05 (s, 2H), 3.52 (t, J=6.0 Hz, 2H), 2.70 (t, J=6.0 Hz, 2H); LCMS (ESI) m/z 281.1 [C₁₈H₂₀N₂O+H]⁺.

### Example 2: Synthesis of Compound 16 Via Synthesis Route A

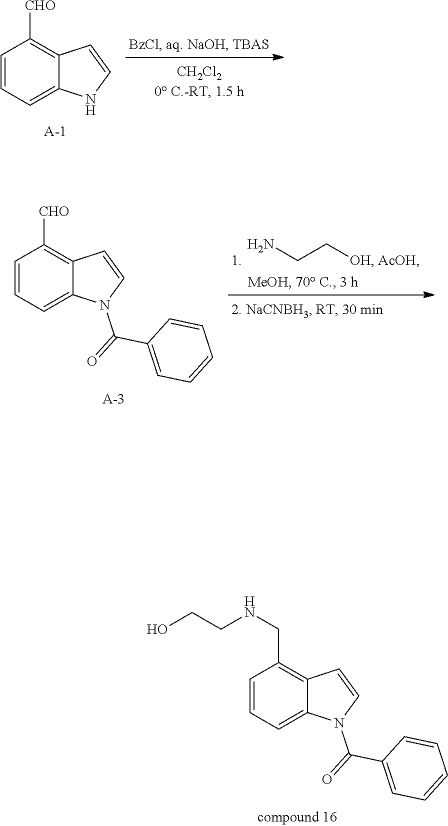

### Synthesis of 1-benzoyl-1H-indole-4-carbaldehyde (A-3)

To a stirred solution of 1H-indole-4-carbaldehyde A-1 (600 mg, 4.13 mmol, 1.0 equiv.) in CH₂Cl₂ (18 mL) at RT, was added tetrabutylammonium hydrogen sulfate (TBAS) (30 mg, 0.08 mmol, 0.02 equiv.) and NaOH (413 mg, 10.3 mmol, 2.5 equiv.) in H₂O (2 mL). The reaction mixture was stirred for 30 min before it was cooled to 0° C. and benzoyl chloride (868 mg, 6.20 mmol, 2.5 equiv.) was added. The resultant mixture was stirred at RT for 1 h and upon completion of reaction by TLC, the reaction mixture was poured into water and extracted with EtOAc (3×200 mL). The combined organic layers were washed with brine solution, dried over Na₂SO₄, concentrated under reduced pressure, and purified by silica gel (100-200 mesh) column chromatography to afford A-3 (600 mg, yield: 58%) as an off-white solid. TLC system: EtOAc/hexane (30:70), R_{f} value=0.6; ¹H NMR (400 MHz, CDCl₃) δ ppm: 10.27 (s, 1H), 8.71 (d, J=8.4 Hz, 1H), 7.81 (dd, J=7.6 Hz, 1.2 Hz, 1H), 7.78-7.75 (m, 2H), 77.67-7.63 (m, 1H), 7.58-7.54 (m, 3H), 7.50 (d, J=4.0 Hz, 1H), 7.46 (d, J=8.0 Hz, 1H); LCMS (ESI) m/z 250.0 [C₁₆H₁₁NO₂+H]⁺.

### Synthesis of (4-(((2-hydroxyethyl)amino)methyl)-1H-indol-1-yl)(phenyl)methanone as formic acid salt (Compound 16)

To a stirred solution of A-3 (150 mg, 0.60 mmol, 1.0 equiv.) in MeOH (2 mL) at RT, was added 2-aminoethan-1-ol (44 mg, 0.72 mmol, 1.2 equiv.) and AcOH (8 mg, 0.12 mmol, 0.2 equiv.). The reaction mixture was stirred at 70° C. for 3 h before it was cooled to 0° C. and NaCNBH₃ (41.6 mg, 0.66 mmol, 1.1 equiv.) was added portion-wise. The resultant mixture was stirred at RT for 30 min and upon completion of reaction by TLC, the reaction mixture was quenched with ice-cold water and extracted with CH₂Cl₂ (2×25 mL). The combined organic layers were dried over Na₂SO₄, concentrated under reduced pressure, and purified by reverse phase column chromatography (Grace) (elution with 22-25% ACN in 0.1% FA in H₂O) to afford compound 16 (formic acid salt) as an off-white solid (21 mg, yield: 10%). TLC system: MeOH/CH₂Cl₂ (10:90), R_{f} value=0.15; melting point range: 135-138° C.; ¹H NMR (400 MHz, DMSO-d₆) δ ppm: 8.24-8.21 (m, 1H), 8.16 (s, 1H; HCOOH), 7.77-7.75 (m, 2H), 7.73-7.69 (m, 1H), 7.64-7.60 (m, 2H), 7.44 (d, J=4.0 Hz, 1H), 7.39-7.37 (m, 2H), 6.98 (d, J=3.6 Hz, 1H), 4.17 (s, 2H), 3.56 (t, J=6.0 Hz, 2H), 2.77 (t→br. s, 2H); LCMS (ESI) m/z 295.1 [C₁₈H₁₈N₂O₂+H]⁺.

### Example 3: Synthesis of Compound 17 Via Synthesis Route A

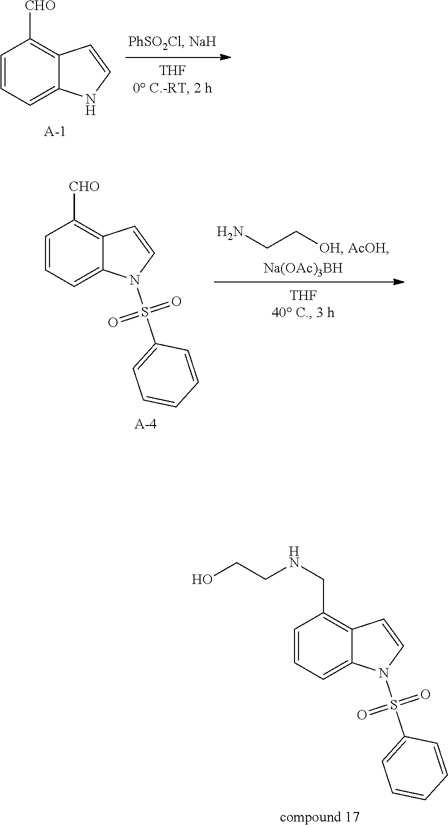

### Synthesis of 1-(phenylsulfonyl)-1H-indole-4-carbaldehyde (A-4)

To a stirred solution of 1H-indole-4-carbaldehyde A-1 (500 mg, 3.44 mmol, 1.0 equiv.) in THE (10 mL) at 0° C., was added NaH (60% dispersion in mineral oil) (344 mg, 8.60 mmol, 2.5 equiv.). The reaction mixture was stirred at 0° C. for 20 min before benzenesulfonyl chloride (910 mg, 5.16 mmol, 1.5 equiv.) was added. The resultant mixture was warmed to RT and was stirred at RT for 2 h. Upon completion of reaction by TLC, the reaction mixture was quenched with sat. NaHCO₃ solution and extracted with EtOAc (3×30 mL). The combined organic layers were washed with brine, dried over Na₂SO₄, and concentrated under reduced pressure to obtain crude material (550 mg) as a brown liquid. The crude material was purified by silica gel (100-200 mesh) column chromatography (elution with 0-15% EtOAc in hexane) to afford A-4 (330 mg, yield: 33%) as a colourless gum. TLC system: EtOAc/hexane (30:70), R_{f} value=0.5; ¹H NMR (400 MHz, CDCl₃) δ ppm: 10.18 (s, 1H), 8.30-8.27 (m, 1H), 7.92-7.87 (m, 2H), 7.77 (d, J=3.6 Hz, 1H), 7.73 (dd, J=7.6 Hz, 1.2 Hz, 1H), 7.57-7.55 (m, 1H), 7.52-7.44 (m, 4H); LCMS (ESI) m/z 286.1 [C₁₅H₁₁NO₃S+H]⁺.

### Synthesis of 2-(((1-(phenylsulfonyl)-1H-indol-4-yl)methyl)amino)ethan-1-ol as formic acid salt (Compound 17)

To a stirred solution of A-4 (300 mg, 1.05 mmol, 1.0 equiv.) in THE (3 mL) at RT, was added 2-aminoethan-1-ol (190 mg, 3.15 mmol, 3.0 equiv.), AcOH (630 mg, 10.5 mmol, 10 equiv.) and Na(OAc)₃BH (669 mg, 3.15 mmol, 3.0 equiv.). The reaction mixture was stirred at 40° C. for 3 h and upon completion of reaction by TLC, the reaction mixture was poured into water and extracted with 10% MeOH in CH₂Cl₂ (2×50 mL). The combined organic layers were washed with brine, dried over Na₂SO₄, and concentrated under reduced pressure. The crude was purified by reverse phase column chromatography (Grace) (elution with 20-30% ACN in 0.05% FA in H₂O) to afford compound 17 (formic acid salt) as a pale-yellow gum (98 mg, yield: 25%). TLC system: MeOH/CH₂Cl₂ (10:90), R_{f} value=0.25; ¹H NMR (400 MHz, DMSO-d₆) δ ppm: 8.25 (s, 1H; HCOOH), 8.00-7.97 (m, 2H), 7.86 (d, J=7.6 Hz, 1H), 7.83 (d, J=3.6 Hz, 1H), 7.71-7.69 (m, 1H), 7.61-7.57 (m, 2H), 7.34-7.28 (m, 2H), 7.03 (dd, J=3.6 Hz, 0.8 Hz, 1H), 4.01 (s, 2H), 3.49 (t, J=5.6 Hz, 2H), 2.66 (t, J=4.6 Hz, 2H); LCMS (ESI) m/z 331.1 [C₁₇H₁₈N₂O₃S+H]⁺.

### Example 4: Synthesis of Compound 21 Via Synthesis Route A

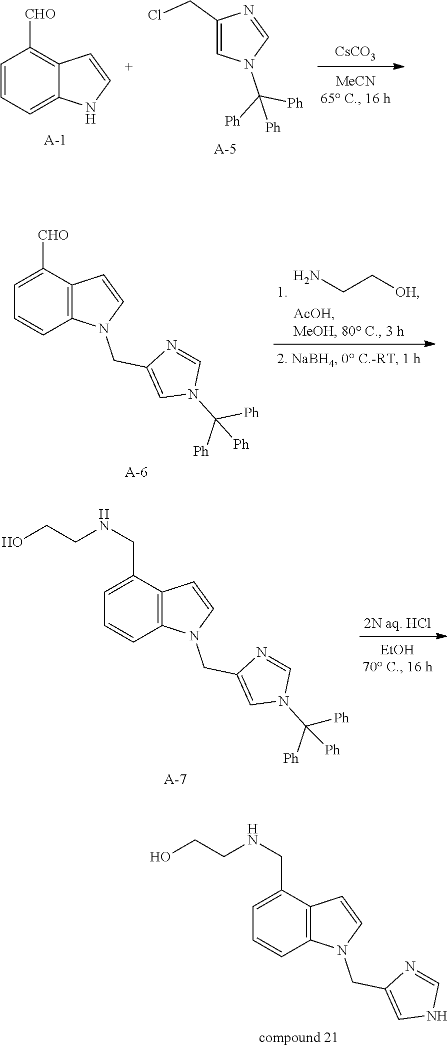

### Synthesis of 1-((1-trityl-1H-imidazol-4-yl)methyl)-1H-indole-4-carbaldehyde (A-6)

To a stirred solution of 1H-indole-4-carbaldehyde A-1 (182 mg, 1.25 mmol, 0.9 equiv.) in MeCN (10 mL) at RT, was added Cs₂CO₃ (905 mg, 2.78 mmol, 2.0 equiv.) and A-5 (500 mg, 1.39 mmol, 1.0 equiv.). The reaction mixture was stirred at 65° C. for 16 h. Upon completion of reaction by TLC, the reaction mixture was diluted with ice-cold water (50 mL) and extracted with EtOAc (2×50 mL). The combined organic layers were washed with brine, dried over anhydrous Na₂SO₄, and concentrated under reduced pressure. The crude obtained was purified by silica gel (100-200 mesh) column chromatography (elution with 40-50% EtOAc in hexane) to afford A-6 as an off-white solid (500 mg, yield: 77%), TLC system: EtOAc/Pet. ether (50:50), R_{f} value=0.2; LCMS (ESI) m/z 468.2 [C₃₂H₂₅N₃O+H]⁺.

### Synthesis of 2-(((1-((1-trityl-1H-imidazol-4-yl)methyl)-1H-indol-4-yl)methyl)amino)ethan-1-ol (A-7)

To a stirred solution of A-6 (500 mg, 1.07 mmol, 1.0 equiv.) in MeOH (5.0 mL) at RT, was added 2-aminoethan-1-ol (0.1 mL, 1.61 mmol, 1.5 equiv.) and AcOH (13 mg, 0.21 mmol, 0.2 equiv.). The reaction mixture was stirred at 70° C. for 16 h before it was cooled to 0° C. and NaBH₄ (101 mg, 2.68 mmol, 2.5 equiv.) was added portion-wise. The resultant mixture was stirred at RT for 1 h. Upon completion of reaction by TLC, the reaction mixture was diluted with ice-cold water (25 mL) and extracted with 10% MeOH in CH₂Cl₂ (2×50 mL). The combined organic layers were washed with brine, dried over anhydrous Na₂SO₄, and concentrated under reduced pressure. The crude obtained was purified by reverse phase column chromatography (Grace) (elution with 30-40% ACN in 0.1% FA in H₂O) to afford A-7 as an off-white solid (200 mg, yield: 36%). TLC system: MeOH/CH₂Cl₂ (10:90), R_{f} value=0.1; LCMS (ESI) m/z 513.4 [C₃₄H₃₂N₄O+H]⁺.

### Synthesis of 2-(((1-((1H-imidazol-4-yl)methyl)-1H-indol-4-yl)methyl)amino)ethan-1-ol as formic acid salt (Compound 21)

To a stirred solution of A-7 (200 mg, 0.39 mmol, 1.0 equiv.) in EtOH (6 mL) at 0° C., was added 2 N aq. HCl (2 mL). The reaction mixture was stirred at 70° C. for 16 h. Upon completion of reaction by TLC, the reaction mixture was concentrated under reduced pressure and purified by Prep-HPLC (formic acid buffer) to afford compound 21 (formic acid salt) as a brown-coloured semi solid (22 mg, yield: 18%). TLC system: MeOH/CH₂Cl₂ (10:90), R_{f} value=0.1; ¹H NMR (400 MHz, DMSO-d₆) δ ppm: 11.91 (br., 1H), 8.21 (s, 1H; HCOOH), 7.55 (s, 1H), 7.51 (d, J=8.0 Hz, 1H), 7.39 (d, J=3.2 Hz, 1H), 7.11-7.03 (m, 3H), 6.55 (d, J=3.2 Hz, 1H), 5.24 (s, 2H), 4.08 (s, 2H), 3.53 (t, J=5.6 Hz, 2H), 2.73 (t, J=5.6 Hz, 2H); LCMS (ESI) m/z 271.2 [C₁₅H₁₈N₄O+H]⁺.

### Example 5: Synthesis of Compound 9 Via Synthesis Route B

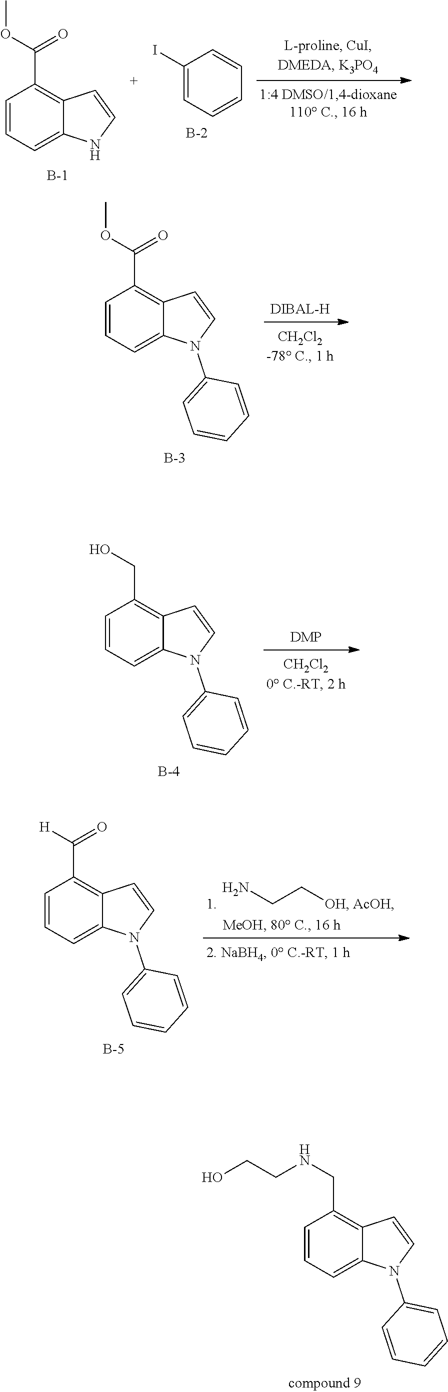

### Synthesis of methyl 1-phenyl-1H-indole-4-carboxylate (B-3)

To a stirred solution of methyl 1H-indole-4-carboxylate B-1 (1.20 g, 6.85 mmol, 1.0 equiv.) in 1:4 DMSO/1,4-dioxane (25 mL) at RT, was added B-2 (515 μL, 4.59 mmol, 0.67 eq), K₃PO₄ (2.91 g, 13.7 mmol, 2.0 equiv.) and L-proline (260 mg, 2.26 mmol, 0.33 equiv.). The mixture was deoxygenated for 5 min before CuI (430 mg, 2.26 mmol, 0.33 equiv.) and N,N-dimethylethylene diamine (0.39 mL, 3.63 mmol, 0.53 equiv.) were added. The resultant mixture was stirred at 110° C. for 16 h and upon completion of reaction by TLC, the reaction mixture was poured into water and extracted with EtOAc (3×100 mL). The combined organic layers were washed with brine, dried over Na₂SO₄, and concentrated under reduced pressure to obtain crude (1.8 g) as a brown liquid. The crude material was purified by silica gel (100-200 mesh) column chromatography (elution with 5% EtOAc in hexane) to afford B-3 (1.53 g, yield: 89%) as a yellow oil. TLC system: EtOAc/hexane (10:90), R_{f} value=0.4; ¹H NMR (400 MHz, CDCl₃) δ ppm: 7.95 (dd, J=7.2 Hz, 0.8 Hz, 1H), 7.71 (d, J=8.4 Hz, 1H), 7.55-7.51 (m, 2H), 7.48-7.45 (m, 3H), 7.41-7.37 (m, 1H), 7.34-7.33 (m 1H), 7.24 (t, J=8.0 Hz, 1H), 4.00 (s, 3H); LCMS (ESI) m/z 252.1 [C₁₆H₁₃NO₂+H]⁺.

### Synthesis of (1-phenyl-1H-indol-4-yl)methanol (B-4)

To a stirred solution of B-3 (1.50 g, 5.97 mmol, 1.0 equiv.) in CH₂Cl₂ (15 mL) at −78° C., was added DIBAL-H (1.5 M in Toluene) (11.9 mL, 17.9 mmol, 3.0 equiv.) drop-wise. The reaction mixture was stirred at −78° C. for 1 h. Upon the completion of reaction by TLC, the reaction mixture was quenched at −78° C. with the addition of MeOH (20 mL). The mixture was warmed to RT, poured into water (30 mL), and extracted with EtOAc (3×20 mL). The combined organic layers were washed with brine, dried over Na₂SO₄, and concentrated under reduced pressure to provide B-4 (1.48 g, crude) as a pale-yellow gum. TLC system: EtOAc/hexane (10:90), R_{f} value=0.3; LCMS (ESI) m/z 224.1 [C₁₅H₁₃NO+H]⁺. The crude material was used in the next step without further purification.

### Synthesis of 1-phenyl-1H-indole-4-carbaldehyde (B-5)

To a stirred solution of B-4 (1.45 g, 6.50 mmol, 1.0 equiv.) in CH₂Cl₂ (15 mL) at 0° C., was added DMP (4.14 g, 9.75 mmol, 1.5 equiv.) portion-wise. The resultant mixture was warmed to RT and stirred at RT for 2 h. Upon the completion of reaction by TLC, the reaction mixture was poured into sat. NaHCO₃ solution (20 mL) and extracted with CH₂Cl₂ (3×20 mL). The combined organic layers were washed with brine, dried over Na₂SO₄, and concentrated under reduced pressure. The crude obtained was purified by silica gel (100-200 mesh) column chromatography (elution with 5% EtOAc in hexane) to afford B-5 (900 mg, yield: 63%) as a yellow oily liquid. TLC system: EtOAc/hexane (20:80), R_{f} value=0.6; LCMS (ESI) m/z 222.1 [C₁₅H₁₁NO+H]⁺.

### Synthesis of 2-(((1-phenyl-1H-indol-4-yl)methyl)amino)ethan-1-ol as formic acid salt (Compound 9)

To a stirred solution of B-5 (250 mg, 1.13 mmol, 1.0 equiv.) in MeOH (5 mL) at RT, was added 2-aminoethan-1-ol (82 μL, 1.36 mmol, 1.2 equiv.) and AcOH (0.05 mL, 0.57 mmol, 0.5 equiv.). The reaction mixture was stirred at 80° C. for 16 h before it was cooled to 0° C. and NaBH₄ (85.5 mg, 2.26 mmol, 2 equiv.) was added portion-wise. The resultant mixture was stirred at RT for 1 h. Upon completion of reaction by TLC, the reaction mixture was concentrated under reduced pressure and purified by reverse phase column chromatography (Grace) (elution with 30% ACN in 0.1% FA in H₂O) to afford compound 9 (formic acid salt) as a pale-yellow gum (48 mg, 14%). TLC system: MeOH/CH₂Cl₂ (10:90), R_{f} value=0.2; ¹H NMR (400 MHz, DMSO-d₆) δ ppm: 8.35 (br. s, 1H; HCOOH), 7.69 (d, J=3.2 Hz, 1H), 7.62-7.57 (m, 4H), 7.50 (d, J=7.6 Hz, 1H), 7.44-7.39 (m, 1H), 7.24-7.18 (m, 2H), 6.91 (d, J=2.8 Hz, 1H), 4.21 (s, 2H), 3.59 (t, J=5.2 Hz, 2H), 2.81 (t, J=4.8 Hz, 2H); LCMS (ESI) m/z 267.1 [C₁₇H₁₈N₂O+H]⁺.

### Example 6: Synthesis of Compound 25 Via Synthesis Route B

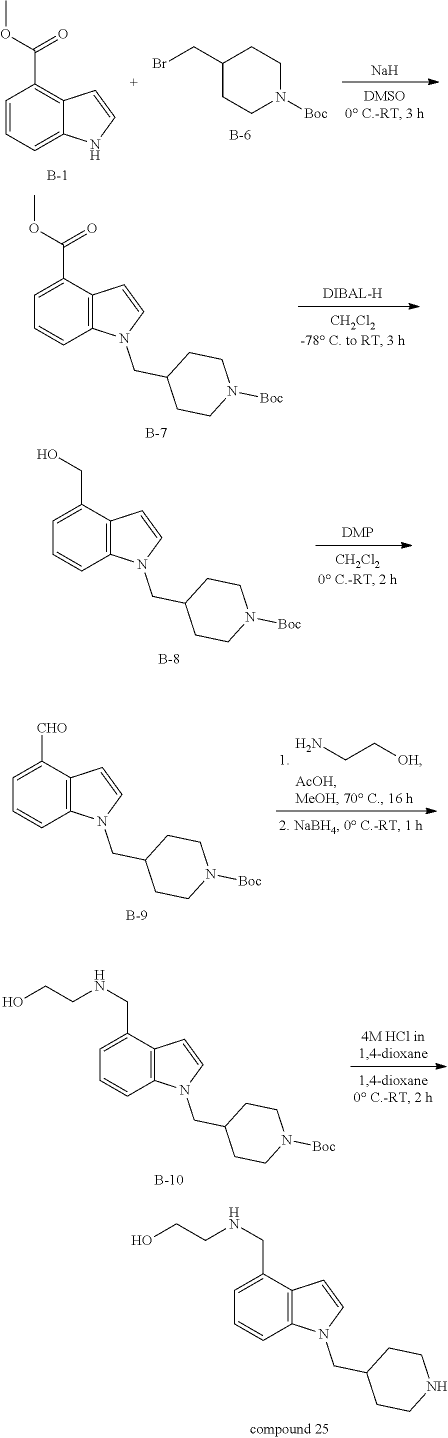

### Synthesis of methyl 1-((1-(tert-butoxycarbonyl)piperidin-4-yl)methyl)-1H-indole-4-carboxylate (B-7)

To a stirred solution of methyl 1H-indole-4-carboxylate B-1 (700 mg, 4.00 mmol, 1.0 equiv.) in DMSO (11 mL) at 0° C., was added NaH (60% dispersion in mineral oil) (400 mg, 10.0 mmol, 2.5 equiv.). The reaction mixture was stirred at 0° C. for 20 min before B-6 (1.33 g, 4.80 mmol, 1.2 equiv.) was added. The resultant mixture was warmed to RT and stirred at RT for 3 h. Upon the completion of reaction by TLC, the reaction mixture was poured into water and extracted with CH₂Cl₂ (2×80 mL). The combined organic layers were washed with brine, dried over Na₂SO₄, and concentrated under reduced pressure. The obtained crude was purified by silica gel (100-200 mesh) column chromatography (elution with 20% EtOAc in hexane) to afford B-7 (380 mg, yield: 26%) as colourless liquid. TLC system: EtOAc/hexane (20:80), R_{f} value=0.2; ¹H NMR (400 MHz, CDCl₃) δ ppm: 7.91 (d, J=7.2 Hz, 1H), 7.52 (d, J=8.0 Hz, 1H), 7.27-7.23 (m, 1H), 7.19 (d, J=3.2 Hz, 1H), 7.12 (d, J=3.2 Hz, 1H), 4.16-4.09 (br. s, 2H), 4.03 (d, J=7.2 Hz, 2H), 3.98 (s, 3H), 2.64-2.57 (m, 2H), 2.02-1.96 (m, 1H), 1.57-1.52 (m, 2H), 1.44 (s, 9H), 1.29-1.22 (m, 2H); LCMS (ESI) m/z 373.2 [C₂₁H₂₈N₂O₄+H]⁺.

### Synthesis of tert-butyl 4-((4-(hydroxymethyl)-1H-indol-1-yl)methyl)piperidine-1-carboxylate (B-8)

To a stirred solution of B-7 (380 mg, 1.02 mmol, 1.0 equiv.) in CH₂Cl₂ (3.8 mL) at −78° C., was added DIBAL-H (1.5 M in Toluene) (1.8 mL, 2.5 mmol, 2.5 equiv.) drop-wise. The resultant mixture was warmed to RT and stirred at RT for 3 h. Upon the completion of reaction by TLC, the reaction mixture was quenched with aq. sat. NH₄Cl solution, filtered through a Celite pad, and extracted with CH₂Cl₂ (2×50 mL). The combined organic layers were washed with brine, dried over Na₂SO₄, and concentrated under reduced pressure to obtain B-8 (350 mg, crude) as a colourless liquid. TLC system: EtOAc/hexane (20:80), R_{f} value=0.1; LCMS (ESI) m/z 327.5 [C₂₀H₂₈N₂O₃—OH]⁻. The crude material was used in the next step without further purification.

### Synthesis of tert-butyl 4-((4-formyl-1H-indol-1-yl)methyl)piperidine-1-carboxylate (B-9)

To a stirred solution of B-8 (350 mg, 1.02 mmol, 1.0 equiv.) in CH₂Cl₂ (3.5 mL) at 0° C., was added DMP (865 mg, 2.04 mmol, 2.0 equiv.). The resultant mixture was warmed to RT and stirred at RT for 2 h. Upon the completion of reaction by TLC, the reaction mixture was quenched with aq. NaHCO₃ solution and extracted with CH₂Cl₂ (2×70 mL). The combined organic layers were washed with brine, dried over Na₂SO₄, and concentrated under reduced pressure. The crude obtained was purified by silica gel (100-200 mesh) column chromatography (elution with 20% EtOAc in hexane) to afford B-9 (240 mg, yield: 69%) as a colourless liquid. TLC system: EtOAc/hexane (20:80), R_{f} value=0.3; ¹H NMR (400 MHz, CDCl₃) δ ppm: 10.25 (s, 1H), 7.64 (d, J=7.2 Hz, 1H), 7.60 (d, J=8.0 Hz, 1H), 7.36 (d, J=8.0 Hz, 1H), 7.30 (d, J=2.8 Hz, 1H), 7.27 (s, 1H), 4.10-4.06 (m, 4H), 2.64-2.58 (m, 2H), 2.02-1.95 (m, 1H), 1.56-1.52 (m, 2H), 1.45 (s, 9H), 1.22-1.20 (m, 2H); LCMS (ESI) m/z 343.2 [C₂₀H₂₆N₂O₃+H]⁺.

### Synthesis of tert-butyl 4-((4-(((2-hydroxyethyl)amino)methyl)-1H-indol-1-yl)methyl)piperidine-1-carboxylate (B-10)

To a stirred solution of B-9 (400 mg, 1.17 mmol, 1.0 equiv.) in MeOH (4.0 mL) at RT, was added 2-aminoethan-1-ol (143 mg, 2.34 mmol, 2.0 equiv.) and AcOH (5 mg, 0.1 mmol, 0.1 equiv.). The mixture was stirred at 70° C. for 16 h before it was cooled to 0° C. and NaBH₄ (111 mg, 2.93 mmol, 2.5 equiv.) was added. The resultant mixture was warmed to RT and stirred at RT for 1 h. Upon the completion of reaction by TLC, the reaction mixture was quenched with ice-cold water and extracted with 10% MeOH in CH₂Cl₂ (2×100 mL). The combined organic layers were washed with brine, dried over Na₂SO₄, and concentrated under reduced pressure. The crude obtained was purified by reverse phase column chromatography (Grace) (elution with 10% ACN in 0.1% FA in H₂O) to afford B-10 (140 mg, yield: 31%) as a colourless liquid. TLC system: MeOH/CH₂Cl₂ (10:90), R_{f} value=0.15; ¹H NMR (400 MHz, CDCl₃) δ ppm: 8.41 (s, 1H; HCOOH), 7.34 (d, J=8.0 Hz, 1H), 7.22 (t, J=7.6 Hz, 1H), 7.17 (d, J=7.2 Hz, 1H), 7.14 (d, J=2.8 Hz, 1H), 6.60 (d, J=2.8 Hz, 1H), 4.36 (s, 2H), 4.10 (br. s, 2H), 3.99 (d, J=7.2 Hz, 2H), 3.79 (t, J=4.8 Hz, 2H), 2.98 (t, J=4.8 Hz, 2H), 2.64-2.58 (m, 2H), 2.00-1.96 (m, 1H), 1.60-1.53 (m, 2H), 1.44 (s, 9H), 1.25-1.16 (m, 2H); LCMS (ESI) m/z 388.3 [C₂₂H₃₃N₃O₃+H]⁺.

### Synthesis of 2-(((1-(piperidin-4-ylmethyl)-1H-indol-4-yl)methyl)amino)ethan-1-ol as bis-formic acid salt (Compound 25)

To a stirred solution of B-10 (105 mg, 0.27 mmol, 1.0 equiv.) in 1,4-Dioxane (1.0 mL) at 0° C., was added 4M HCl in 1,4-Dioxane (1.0 mL). The mixture was warmed to RT and stirred at RT for 2 h. Upon the completion of reaction by TLC, the reaction mixture was concentrated under reduced pressure and washed with n-pentane to afford crude compound. The crude compound was neutralized with sat. aq. NaHCO₃ solution and extracted with 10% MeOH in CH₂Cl₂ (3×30 mL). The combined organic layers were evaporated and purified by reverse phase column chromatography (Grace) (elution with 2-10% ACN in 0.1% FA in H₂O) to afford compound 25 (bis-formic acid salt) as a light-yellow gummy liquid (25 mg, yield: 24%). TLC system: MeOH/CH₂Cl₂ (10:90), R_{f} value=0.1; ¹H NMR (400 MHz, DMSO-d₆) δ ppm: 8.32 (s, 2H; 2HCOOH), 7.38 (d, J=8.0 Hz, 1H), 7.34 (d, J=3.2 Hz, 1H), 7.09 (t, J=7.6 Hz, 1H), 7.02 (d, J=7.2 Hz, 1H), 6.55 (d, J=2.8 Hz, 1H), 4.08 (d, J=6.8 Hz, 2H), 4.00 (s, 2H), 3.50 (t, J=5.6 Hz, 2H), 3.15-3.12 (m, 2H), 2.66 (m, 4H), 2.02 (br. s, 1H), 1.58-1.55 (m, 2H), 1.36-1.28 (m, 2H); LCMS (ESI) m/z 288.1 [C₁₇H₂₅N₃O+H]⁺.

### Example 7: Synthesis of Compound 37 Via Synthesis Route B

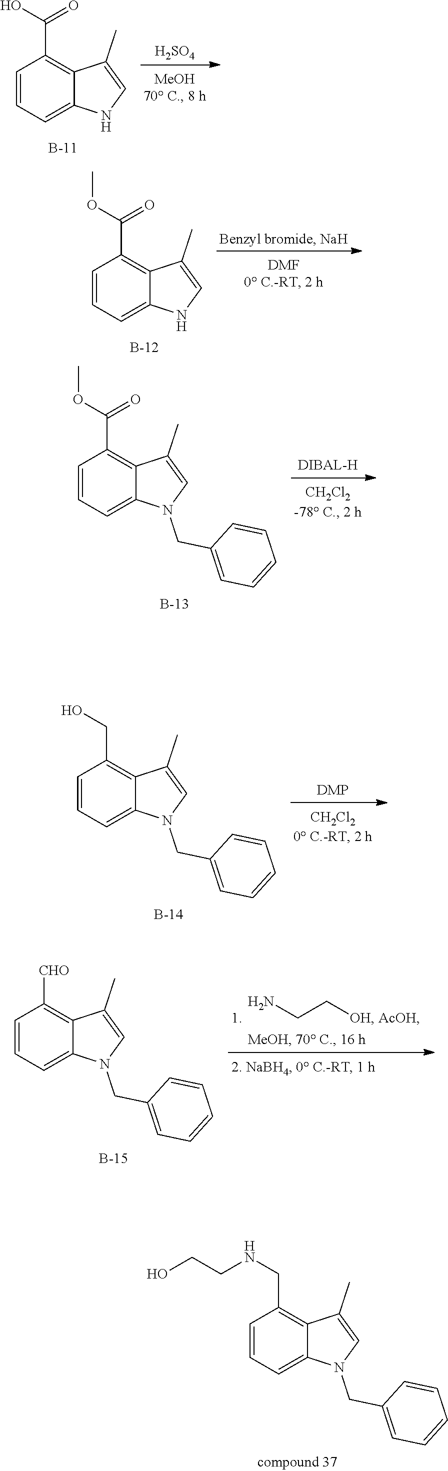

### Synthesis of methyl 3-methyl-1H-indole-4-carboxylate (B-12)

To a stirred solution of 3-methyl-1H-indole-4-carboxylic acid B-11 (500 mg, 2.85 mmol, 1.0 equiv.) in MeOH (25 mL) at RT, was added H₂SO₄ (0.3 mL, 5.71 mmol, 2.0 equiv.) dropwise. The resultant mixture was stirred at 70° C. for 8 h. Upon the completion of reaction by TLC, the reaction mixture was quenched with sat. NaHCO₃ solution and extracted with CH₂Cl₂ (3×20 mL). The combined organic layers were washed with brine, dried over Na₂SO₄, concentrated under reduced pressure, and purified by silica gel (100-200 mesh) column chromatography (elution with 20% EtOAc in hexane) to afford B-12 (500 mg, yield: 93%) as a yellow gummy liquid. TLC system: EtOAc/hexane (50:50), R_{f} value=0.3; ¹H NMR (400 MHz, CDCl₃) δ ppm: 8.09 (br. s, 1H), 7.63 (dd, J=7.6, 1.2 Hz, 1H), 7.50 (dd, J=8.0 Hz, 1.2 Hz, 1H), 7.18 (t, J=8.0 Hz, 1H), 7.10 (s, 1H), 3.96 (s, 3H), 2.41 (s, 3H); LCMS (ESI) m/z 190.1 [C₁₁H₁₁NO₂+H]⁺.

### Synthesis of methyl 1-benzyl-3-methyl-1H-indole-4-carboxylate (B-13)

To a stirred solution of B-12 (500 mg, 2.64 mmol, 1.0 equiv.) in DMF (10 mL) at 0° C., was added NaH (60% dispersion in mineral oil) (158 mg, 3.96 mmol, 1.5 equiv.). The mixture was stirred at 0° C. for 5 min before benzyl bromide (0.34 mL, 2.90 mmol, 1.1 equiv.) was added. The resultant mixture was warmed to RT and stirred at RT for 2 h. Upon the completion of reaction by TLC, the reaction mixture was poured into water and extracted with CH₂Cl₂ (3×20 mL). The combined organic layers were washed with brine, dried over Na₂SO₄, and concentrated under reduced pressure to obtain B-13 (600 mg, yield: 81%) as a yellow gummy liquid in its crude. TLC system: EtOAc/hexane (20:80), R_{f} value=0.5; ¹H NMR (400 MHz, CDCl₃) δ ppm: 7.60 (dd, J=7.6 Hz, 1.2 Hz, 1H), 7.40 (dd, J=8.0 Hz, 1.2 Hz, 1H), 7.31-7.26 (m, 3H), 7.15 (t, J=8.0 Hz, 1H), 7.09-7.07 (m, 2H), 7.02 (s, 1H), 5.29 (s, 2H), 3.96 (s, 3H), 2.41 (s, 3H), LCMS (ESI) m/z 280.1 [C₁₈H₁₇NO₂+H]⁺.

### Synthesis of (1-benzyl-3-methyl-1H-indol-4-yl)methanol (B-14)

Compound B-14 was obtained following the ester reduction procedure similar to that for the synthesis of compound B-8, utilizing DIBAL-H and reaction conditions of addition at −78° C. and stirring at RT for 2 h. B-14 was obtained in 64% yield as a yellow solid in its crude. TLC system: EtOAc/hexane (20:80), R_{f} value=0.2; LCMS (ESI) m/z 252.1 [C₁₇H₁₇NO+H]⁺.

### Synthesis of 1-benzyl-3-methyl-1H-indole-4-carbaldehyde (B-15)

Compound B-15 was obtained following the alcohol oxidation procedure similar to that for the synthesis of compound B-9, utilizing DMP and reaction conditions of addition at 0° C. and stirring at RT for 2 h. B-15 was obtained in 74% yield as a yellow oily liquid following silica gel column chromatography (elution with 5% EtOAc in hexane). TLC system: EtOAc/hexane (10:90), R_{f} value=0.5; ¹H NMR (400 MHz, CDCl₃) δ ppm: 10.65 (s, 1H), 7.76 (dd, J=7.6 Hz, 0.8 Hz, 1H), 7.50 (dd, J=8.0 Hz, 0.8 Hz, 1H), 7.33-7.22 (m, 4H), 7.11-7.09 (m, 3H), 5.32 (s, 2H), 2.58 (s, 3H); LCMS (ESI) m/z 250.1 [C₁₇H₁₅NO+H]⁺.

### Synthesis of 2-(((1-benzyl-3-methyl-1H-indol-4-yl)methyl)amino)ethan-1-ol as formic acid salt (Compound 37)

Compound 37 was obtained following the reductive amination procedure similar to that for the synthesis of compound B-10, utilizing 2-aminoethan-1-ol with NaBH₄ and reaction conditions of 0° C. to RT for 1 h. Compound 37 (formic acid salt) was obtained in 34% yield as an off-white solid following reverse phase column chromatography (Grace) (elution with 20% MeCN in 0.1% formic acid in H₂O). TLC system: MeOH/CH₂Cl₂ (10:90), R_{f} value=0.1; melting point range: 131-134° C.; ¹H NMR (400 MHz, DMSO-d₆) δ ppm: 8.28 (br. s, 1H; HCOOH), 7.34 (d, J=8.0 Hz, 1H), 7.31-7.28 (m, 2H), 7.24-7.21 (m, 2H), 7.18 (d, J=8.0 Hz, 2H), 7.05-6.98 (m, 2H), 5.33 (s, 2H), 4.19 (s, 2H), 3.57 (t, J=5.6 Hz, 2H), 2.81 (t, J=5.6 Hz, 2H), 2.45 (s, 3H); LCMS (ESI) m/z 295.2 [C₁₉H₂₂N₂O+H]⁺.

### Example 8: Synthesis of Compound 45 Via Synthesis Route B

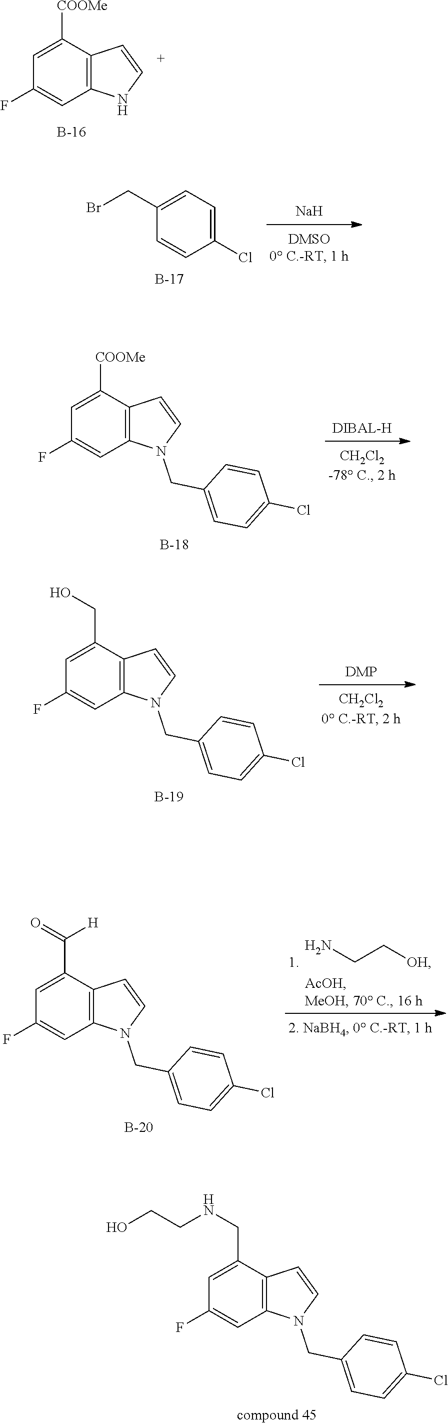

### Synthesis of methyl 1-(4-chlorobenzyl)-6-fluoro-1H-indole-4-carboxylate (B-18)

To a stirred solution of B-16 (500 mg, 2.59 mmol, 1.0 equiv.) in DMSO (7.5 mL) at 0° C., was added NaH (60% dispersion in mineral oil) (259 mg, 6.47 mmol, 2.5 equiv.). The reaction mixture was stirred at RT for 30 min, before it was cooled to 0° C., and B-17 (1.32 g, 6.47 mmol, 2.5 equiv.) was added. The reaction mixture was warmed to RT and stirred at RT for 1 h. Upon the completion of the reaction by TLC, the reaction mixture was quenched with ice-cold water and extracted with CH₂Cl₂ (3×50 mL). The combined organic layers were washed with brine, dried over Na₂SO₄, concentrated under reduced pressure, and purified by silica gel (60-120 mesh) column chromatography (elution with 10% EtOAc in hexanes) to afford B-18 (779 mg, yield: 95%) as a pale yellow solid. TLC system: EtOAc/hexane (20:80), R_{f} value=0.5; ¹H NMR (400 MHz, CDCl₃) δ ppm: 7.64 (dd, J=10.4 Hz, 2.4 Hz, 1H), 7.30-7.27 (m, 2H), 7.24 (d, J=3.2 Hz, 1H), 7.17 (dd, J=3.2 Hz, 0.8 Hz, 1H), 7.12-7.09 (m, 1H), 7.00 (d, J=6.4 Hz, 2H), 5.27 (s, 2H), 3.99 (s, 3H); LCMS (ESI) m/z 318.1 [C₁₇H₁₃ClFNO₂+H]⁺.

### Synthesis of (1-(4-chlorobenzyl)-6-fluoro-1H-indol-4-yl)methanol (B-19)

Compound B-19 was obtained following the ester reduction procedure similar to that for the synthesis of compound B-8, utilizing DIBAL-H and reaction conditions of addition at −78° C. and stirring at RT for 2 h. B-19 was obtained in 81% yield as a light yellow liquid. TLC system: EtOAc/hexane (20:80), R_{f} value=0.3; ¹H NMR (400 MHz, CDCl₃) δ ppm: 7.31-7.27 (m, 2H), 7.12 (d, J=3.2 Hz, 1H), 7.01 (d, J=8.4 Hz, 2H), 6.95 (dd, J=10.0 Hz, 2.0 Hz, 1H), 6.84 (dd, J=9.6 Hz, 2.0 Hz, 1H), 6.61 (d, J=3.2 Hz, 1H), 5.24 (s, 2H), 4.97 (d, J=5.2 Hz, 2H), 1.77 (t, J=5.6 Hz, 1H); LCMS (ESI) m/z 290.1 [C₁₆H₁₃ClFNO+H]⁺.

### Synthesis of 1-(4-chlorobenzyl)-6-fluoro-1H-indole-4-carbaldehyde (B-20)

Compound B-20 was obtained following the alcohol oxidation procedure similar to that for the synthesis of compound B-9, utilizing DMP and reaction conditions of addition at 0° C. and stirring at RT for 2 h. B-20 was obtained in 40% yield as a green solid following silica gel column chromatography (elution with 10% EtOAc in hexane). TLC system: EtOAc/hexane (10:90), R_{f} value=0.4; ¹H NMR (400 MHz, CDCl₃) δ ppm: 10.22 (s, 1H), 7.40 (dd, J=9.2 Hz, 2.0 Hz, 1H), 7.32-7.28 (m, 4H), 7.18 (dd, J=9.2 Hz, 2.0 Hz, 1H), 7.01 (d, J=8.8 Hz, 2H), 5.30 (s, 2H); LCMS (ESI) m/z 288.0 [C₁₆H₁₁ClFNO+H]⁺.

### Synthesis of 2-(((1-(4-chlorobenzyl)-6-fluoro-1H-indol-4-yl)methyl)amino)ethan-1-ol as formic acid salt (Compound 45)

Compound 45 was obtained following the reductive amination procedure similar to that for the synthesis of compound B-10, utilizing 2-aminoethan-1-ol with NaBH₄ and reaction conditions of 0° C. to RT for 1 h. Compound 45 (formic acid salt) was obtained in 45% yield as an off-white solid following reverse phase column chromatography (Grace) (elution with 20% MeCN in 0.1% formic acid in H₂O). TLC system: MeOH/CH₂Cl₂ (10:90), R_{f} value=0.2; melting point range: 111-114° C.; ¹H NMR (400 MHz, DMSO-d₆) δ ppm: 8.22 (s, 1H; HCOOH), 7.50 (d, J=3.2 Hz, 1H), 7.38 (dd, J=6.4, 2.0 Hz, 2H), 7.26-7.22 (m, 3H), 6.96 (dd, J=10.8, 2.4 Hz, 1H), 6.62 (dd, J=3.2, 0.8 Hz, 1H), 5.39 (s, 2H), 4.04 (s, 2H), 3.52 (t, J=5.6 Hz, 2H), 2.69 (t, J=5.6 Hz, 2H); LCMS (ESI) m/z 333.1 [C₁₈H₁₈ClFN₂O+H]⁺.

### Example 9: Synthesis of Compound 112 Via Synthesis Route B

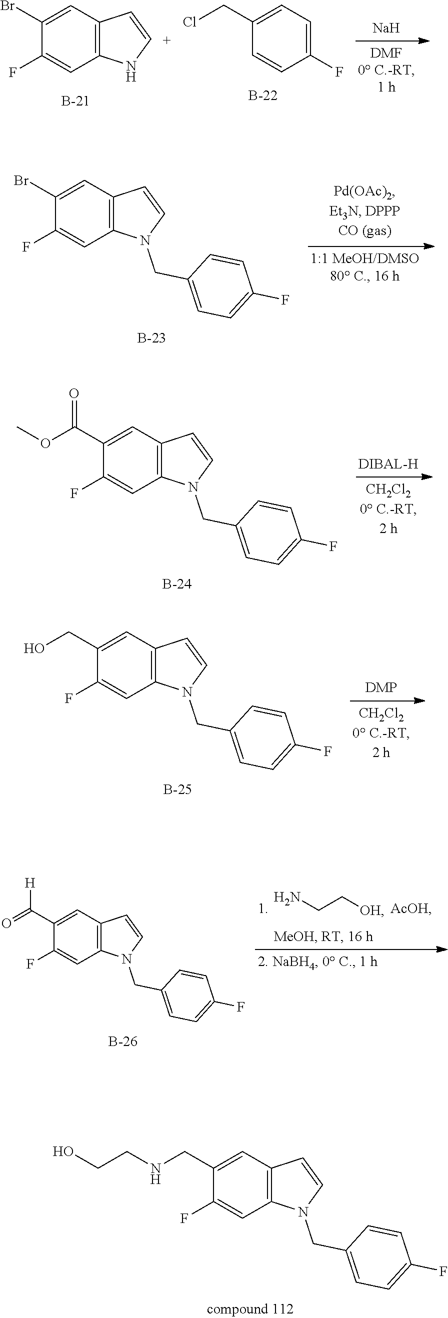

### Synthesis of 5-bromo-6-fluoro-1-(4-fluorobenzyl)-1H-indole (B-23)

To a stirred solution of B-21 (1.00 g, 4.67 mmol, 1.0 equiv.) in DMF (10 mL) at 0° C., was added NaH (60% dispersion in mineral oil) (374 mg, 9.34 mmol, 2.0 equiv.). The reaction mixture was stirred at 0° C. for 20 min, before B-22 (878 mg, 6.07 mmol, 1.3 equiv.) was added. The resultant mixture was warmed to RT and stirred at RT for 1 h. Upon the completion of the reaction by TLC, the reaction mixture was diluted with ice-cold water (20 mL) and extracted with EtOAc (3×30 mL). The combined organic layers were washed with brine, dried over anhydrous Na₂SO₄, filtered, and concentrated under reduced pressure. The crude material obtained was purified by silica gel (60-120 mesh) column chromatography (elution with 5% EtOAc in hexane) to afford B-23 (1.00 g, yield: 66%) as an off-white solid. TLC system: EtOAc/hexane (20:80), R_{f} value=0.6; ¹H NMR (400 MHz, CDCl₃) δ ppm: 7.77 (d, J=6.8 Hz, 1H), 7.11 (d, J=3.2 Hz, 1H), 7.07-6.97 (m, 5H), 6.48 (dd, J=3.2 Hz, 0.8 Hz, 1H), 5.22 (s, 2H).

### Synthesis of methyl 6-fluoro-1-(4-fluorobenzyl)-1H-indole-5-carboxylate (B-24)

To a stirred solution of B-23 (1.00 g, 3.10 mmol, 1.0 equiv.) in 1:1 DMSO/MeOH (20 mL) at RT, was added Et₃N (4.32 mL, 31.0 mmol, 10.0 equiv.), followed by DPPP (256 mg, 0.620 mmol, 0.2 equiv.). The reaction mixture was degassed with nitrogen for 10 min, before Pd(OAc)₂ (69.6 mg, 0.310 mmol, 0.1 equiv.) was added. The mixture was degassed again with nitrogen for 5 min and the resultant reaction mixture was kept under CO atmosphere (100 psi) in a steel bomb and stirred at 80° C. for 16 h. Upon the completion of the reaction by TLC, the reaction mixture was diluted with H₂O (20 mL) and extracted with EtOAc (2×30 mL). The combined organic layers were dried over anhydrous Na₂SO₄, filtered and concentrated under reduced pressure. The crude material obtained was purified by silica gel (60-120 mesh) column chromatography (elution with 15% EtOAc in hexane) to afford B-24 (800 mg, yield: 86%) as an off-white solid. TLC system: EtOAc/hexane (20:80), R_{f} value=0.1; LCMS (ESI) m/z 302.0 [C₁₇H₁₃F₂NO₂+H]⁺.

### Synthesis of (6-fluoro-1-(4-fluorobenzyl)-1H-indol-5-yl)methanol (B-25)

Compound B-25 was obtained following the ester reduction procedure similar to that for the synthesis of compound B-8, utilizing DIBAL-H (1.5 M in toluene, 3.0 equiv.) and reaction conditions of stirring at 0° C. for 2 h. B-25 was obtained in 93% yield as an off-white solid. TLC system: EtOAc/hexane (30:70), R_{f} value=0.3; ¹H NMR (400 MHz, CDCl₃) δ ppm: 7.54 (d, J=7.2 Hz, 1H), 7.03 (d, J=3.2 Hz, 1H), 7.01-6.97 (m, 2H), 6.94-6.84 (m, 3H), 6.46 (d, J=4.0 Hz, 1H), 5.16 (s, 2H), 4.74 (s, 2H).

### Synthesis of 6-fluoro-1-(4-fluorobenzyl)-1H-indole-5-carbaldehyde (B-26)

Compound B-26 was obtained following the alcohol oxidation procedure similar to that for the synthesis of compound B-9, utilizing DMP (1.3 equiv.) and reaction conditions of addition at 0° C. and stirring at RT for 2 h. B-26 was obtained in 72% yield as a brown solid following silica gel column chromatography (elution with 10% EtOAc in hexane). TLC system: EtOAc/hexane (20:80), R_{f} value=0.5; ¹H NMR (400 MHz, CDCl₃) δ ppm: 10.33 (s, 1H), 8.17 (d, J=6.8 Hz, 1H), 7.16 (d, J=3.2 Hz, 1H), 7.11-7.07 (m, 2H), 7.05-6.93 (m, 3H), 6.67-6.65 (m, 1H), 5.25 (s, 2H).

### Synthesis of 2-(((6-fluoro-1-(4-fluorobenzyl)-1H-indol-5-yl)methyl)amino)ethan-1-ol (Compound 112)

To a stirred solution of B-26 (100 mg, 0.369 mmol, 1.0 equiv.) in MeOH (1.0 mL) at 0° C., were added 2-aminoethan-1-ol (29.0 μL, 0.480 mmol, 1.3 equiv.) and AcOH (42.2 μL, 0.738 mmol, 2.0 equiv.). The reaction mixture was stirred at RT for 16 h, before it was cooled to 0° C., and NaBH₄ (21.0 mg, 0.554 mmol, 1.5 equiv.) was added portion wise. The resultant mixture was stirred at 0° C. for 1 h. Upon the completion of the reaction by TLC, the reaction mixture was quenched with ice-cold water and solid precipitated out. The solid was filtered, washed with ether, pentane and dried to afford compound 112 (81 mg, yield: 69%) as an off-white solid. TLC system: MeOH/CH₂Cl₂ (10:90); R_{f} value=0.2; ¹H NMR (400 MHz, DMSO-d₆) δ ppm: 7.53 (d, J=7.6 Hz, 1H), 7.47 (d, J=3.2 Hz, 1H), 7.32-7.24 (m, 3H), 7.14 (t, J=8.8 Hz, 2H), 6.45 (d, J=2.8 Hz, 1H), 5.36 (s, 2H), 4.45 (t, J=5.2 Hz, 1H), 3.76 (s, 2H), 3.45 (q, J=5.2 Hz, 2H), 2.57 (t, J=5.6 Hz, 2H), 1.97 (br. s, 1H); LCMS (ESI) m/z 317.2 [C₁₈H₁₈F₂N₂O+H]⁺.

### Example 10: Synthesis of Compound 4 Via Synthesis Route C

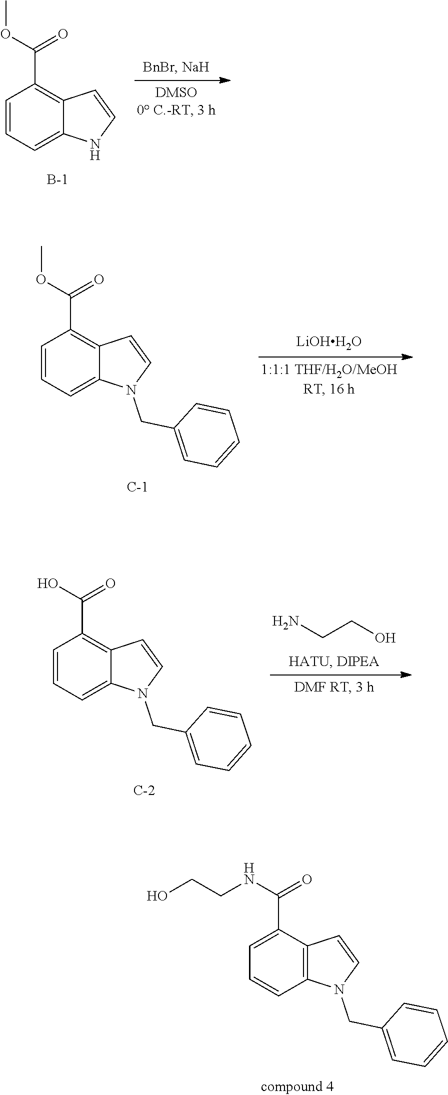

### Synthesis of methyl 1-benzyl-1H-indole-4-carboxylate (C-1)

Compound C-1 was obtained following the alkylation procedure similar to that for the synthesis of compound B-7, utilizing benzyl bromide and reaction conditions of 0° C. to RT for 3 h. C-1 was obtained in 83% yield as a colourless liquid following silica gel column chromatography (elution with 10% EtOAc in hexane). TLC system: EtOAc/hexane (20:80), R_{f} value=0.4; ¹H NMR (400 MHz, CDCl₃) δ ppm: 7.90 (dd, J=7.6 Hz, 0.8 Hz, 1H), 7.47 (d, J=7.6 Hz, 1H), 7.31-7.24 (m, 4H), 7.20-7.18 (m, 2H), 7.09-7.06 (m, 2H), 5.36 (s, 2H), 3.98 (s, 3H); LCMS (ESI) m/z 266.1 [C₁₇H₁₅NO₂+H]⁺.

### Synthesis of 1-benzyl-1H-indole-4-carboxylic acid (C-2)

To a stirred solution of C-1 (750 mg, 2.83 mmol, 1.0 equiv.) in 1:1:1 THF/H₂O/MeOH (9 mL) at RT, was added LiOH·H₂O (596 mg, 14.2 mmol, 5.0 equiv.). The resultant mixture was stirred at RT for 16 h. Upon the completion of reaction by TLC, the reaction mixture was concentrated, acidified with 1N HCl (pH 5), and extracted with 10% MeOH in CH₂Cl₂ (3×20 mL). The combined organic layers were dried over anhydrous Na₂SO₄, filtered, and concentrated to afford C-2 as off-white solid (500 mg, yield: 70%) in its crude. TLC system: MeOH/CH₂Cl₂ (5:95), R_{f} value=0.2; ¹H NMR (400 MHz, CDCl₃) δ ppm: 8.02 (d, J=6.8 Hz, 1H), 7.52 (d, J=8.0 Hz, 1H), 7.33-7.25 (m, 5H), 7.23 (d, J=8.0 Hz, 1H), 7.11-7.09 (m, 2H), 5.39 (s, 2H); LCMS (ESI) m/z 252.1 [C₁₆H₁₃NO₂+H]⁺.

### Synthesis of 1-benzyl-N-(2-hydroxyethyl)-1H-indole-4-carboxamide (Compound 4)

To a stirred solution of C-2 (400 mg, 1.59 mmol, 1.0 equiv.) in DMF (4 mL) at RT, was added HATU (908 mg, 2.39 mmol, 1.5 equiv.) and DIPEA (934 μL, 3.98 mmol, 2.5 equiv.). The mixture was stirred at RT for 20 min before 2-aminoethan-1-ol (192 μL, 3.18 mmol, 2.0 equiv.) was added. The resultant mixture was stirred at RT for additional 3 h and upon completion of reaction, the reaction mixture was diluted with water and extracted with 10% MeOH in CH₂Cl₂ (3×20 mL). The combined organic layers were dried over anhydrous Na₂SO₄, filtered, and concentrated. The crude obtained was purified by reverse phase column chromatography (Grace) (elution with 20% ACN in 0.1% FA in H₂O) to afford compound 4 as an off-white solid (158 mg, yield: 34%). TLC system: MeOH/CH₂Cl₂ (10:90), R_{f} value=0.2; melting point range: 111-114° C.; ¹H NMR (400 MHz, DMSO-d₆) δ ppm: 8.12 (t, J=5.6 Hz, 1H), 7.60-7.58 (m, 2H), 7.41 (d, J=7.2 Hz, 1H), 7.32-7.28 (m, 2H), 7.25-7.21 (m, 1H), 7.18-7.12 (m, 3H), 6.89 (d, J=3.2 Hz, 1H), 5.46 (s, 2H), 4.73 (t, J=5.6 Hz, 1H), 3.54 (q, J=6.0 Hz, 2H), 3.37 (q, J=6.0 Hz, 2H); LCMS (ESI) m/z 295.2 [C₁₈H₁₈N₂O₂+H]⁺.

### Example 11: Synthesis of Compound 5 Via Synthesis Route D

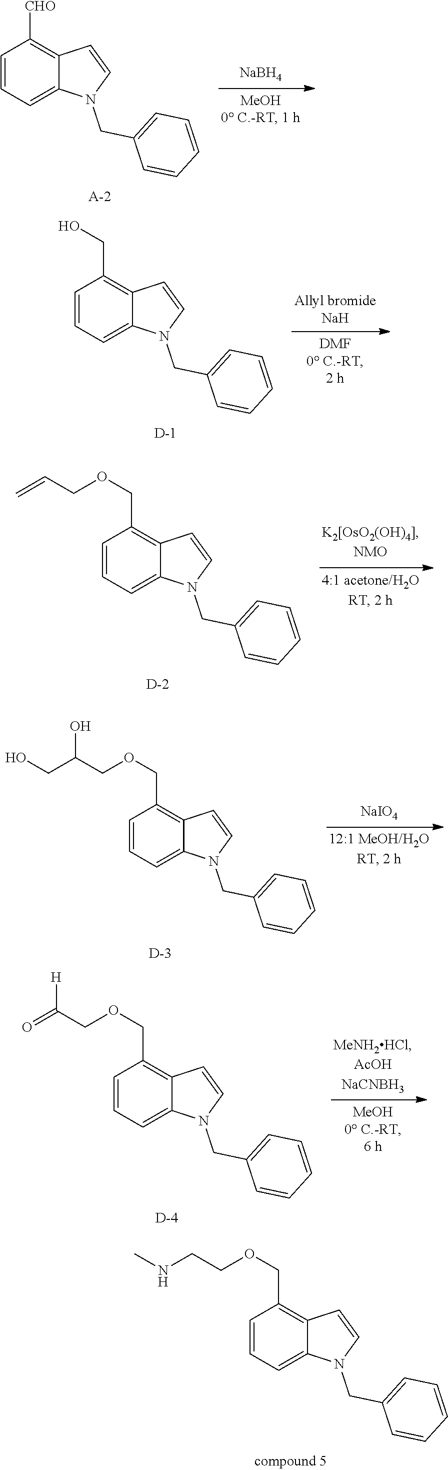 [SMILES: O=Cc1cccc2c1ccn2Cc1ccccc1 ; OCc1cccc2c1ccn2Cc1ccccc1 ; C=CCOCc1cccc2c1ccn2Cc1ccccc1 ; OCC(O)COCc1cccc2c1ccn2Cc1ccccc1 ; O=CCOCc1cccc2c1ccn2Cc1ccccc1 ; CNCCOCc1cccc2c1ccn2Cc1ccccc1]

### Synthesis of (1-benzyl-1H-indol-4-yl)methanol (D-1)

To a stirred solution of A-2 (1.0 g, 4.25 mmol, 1.0 equiv.) in MeOH (20 mL) at 0° C., was added NaBH₄ (402 mg, 10.6 mmol, 2.5 equiv.) portion-wise. The resultant mixture was warmed to RT and stirred at RT for 1 h. Upon the completion of reaction by TLC, the reaction mixture was diluted with ice-cold water (50 mL) and extracted with CH₂Cl₂ (3×50 mL). The combined organic layers were washed with brine, dried over Na₂SO₄, and concentrated under reduced pressure to afford D-1 (750 mg, crude) as a yellow solid. TLC system: EtOAc/hexane (30:70), R_{f} value=0.6; LCMS (ESI) m/z 238.1 [C₁₆H₁₅NO+H]⁺. The crude compound was used directly in the next step without further purification.

### Synthesis of 4-((allyloxy)methyl)-1-benzyl-1H-indole (D-2)

To a stirred solution of D-1 (500 mg, 2.10 mmol, 1.0 equiv.) in DMF (5 mL) at 0° C., was added NaH (60% dispersion in mineral oil) (211 mg, 5.27 mmol, 2.5 equiv.). The mixture was removed from the ice bath and stirred at RT for 20 min before it was cooled back to 0° C. and 3-bromoprop-1-ene (455 μL, 5.27 mmol, 2.5 equiv.) was added. The resultant mixture was warmed to RT and stirred at RT for 2 h. Upon completion of reaction by TLC, the reaction mixture was quenched with ice-cold water (50 mL) and extracted with CH₂Cl₂ (2×100 mL). The combined organic layers were washed with brine, dried over Na₂SO₄, and concentrated under reduced pressure to afford D-2 (520 mg, crude) as a brown gummy liquid. TLC system: EtOAc/hexane (30:70), R_{f} value=0.6; LCMS (ESI) m/z 278.1 [C₁₉H₁₉NO+H]⁺. The crude compound was used directly in the next step without further purification.

### Synthesis of 3-((1-benzyl-1H-indol-4-yl)methoxy)propane-1,2-diol (D-3)

To a stirred solution of D-2 (520 mg, 1.87 mmol, 1.0 equiv.) in 4:1 acetone/water (12.5 mL) at RT, was added K₂[OsO₂(OH)₄](70 mg, 0.19 mmol, 0.1 equiv.) and NMO [50% in water, (285 mg, 2.43 mmol, 1.3 equiv.)]. The reaction mixture stirred at RT for 2 h. Upon the completion of reaction by TLC, the reaction mixture was diluted with water (50 mL) and extracted with EtOAc (3×50 mL). The combined organic layers were washed with brine, dried over Na₂SO₄, and concentrated under reduced pressure. The crude obtained was purified by silica gel (100-200 mesh) column chromatography (elution with 35% EtOAc in hexane) to afford D-3 (250 mg, yield: 43%) as a light green gummy liquid. TLC system: EtOAc/hexane (50:50), R_{f} value=0.2; ¹H NMR (400 MHz, CDCl₃) δ ppm: 7.32-7.27 (m, 4H), 7.17-7.11 (m, 4H), 7.06 (d, J=7.2 Hz, 1H), 6.64 (dd, J=3.2 Hz, 0.4 Hz, 1H), 5.34 (s, 2H), 4.86 (s, 2H), 3.88 (br. s, 1H), 3.68-3.57 (m, 4H), 2.57 (br. s, 1H), 2.03 (br. s, 1H); LCMS (ESI) m/z 310.1 [C₁₉H₂₁NO₃—H]⁻.

### Synthesis of 2-((1-benzyl-1H-indol-4-yl)methoxy)acetaldehyde (D-4)

To a stirred solution of D-3 (250 mg, 0.80 mmol, 1.0 equiv.) in MeOH (6 mL) and water (0.5 mL) at RT, was added NaIO₄ (275 mg, 1.28 mmol, 1.6 equiv.). The resultant mixture was stirred at RT for 2 h. Upon the completion of reaction by TLC, the reaction mixture was diluted with water (50 mL) and extracted with CH₂Cl₂ (3×30 mL). The organic layers were washed with brine, dried over Na₂SO₄, concentrated under reduced pressure, and purified by silica gel (100-200 mesh) column chromatography (elution with 20% EtOAc in hexane) to afford D-4 (120 mg, yield: 53%) as an oily liquid. TLC system: EtOAc/hexane (50:50), R_{f} value=0.4; ¹H NMR (400 MHz, CDCl₃) δ ppm: 9.71 (s, 1H), 7.34-7.27 (m, 3H), 7.19-7.08 (m, 6H), 6.70 (dd, J=4.4 Hz, 0.8 Hz, 1H), 5.34 (s, 2H), 4.96 (s, 2H), 4.12 (s, 2H); LCMS (ESI) m/z 280.1 [C₁₈H₁₇NO₂+H]⁺.

### Synthesis of 2-((1-benzyl-1H-indol-4-yl)methoxy)-N-methylethan-1-amine (Compound 5)

To a stirred solution of D-4 (60 mg, 0.21 mmol, 1.0 equiv.) in MeOH (1 mL) at RT, was added MeNH₂·HCl (43.2 mg, 0.64 mmol, 3.0 equiv.) and AcOH (1.2 μL, 0.02 mmol, 0.1 equiv.). The reaction mixture was cooled to 0° C. before NaCNBH₃ (40.2 mg, 0.64 mmol, 3.0 equiv.) was added. The resultant mixture was then warmed to RT and stirred at RT for 6 h. Upon the completion of reaction by TLC, the reaction mixture was quenched with ice-cold water and extracted with 10% MeOH in CH₂Cl₂ (2×15 mL). The combined organic layers were dried over Na₂SO₄, concentrated under reduced pressure, and purified by reverse phase column chromatography (Grace) (elution with 19-20% ACN in 0.1% FA in H₂O) to afford compound 5 (30 mg, yield: 49%) as an off-white gummy solid. TLC system: MeOH/CH₂Cl₂ (10:90), R_{f} value=0.5; ¹H NMR (400 MHz, DMSO-d₆) δ ppm: 7.53 (d, J=3.2 Hz, 1H), 7.41 (d, J=8.0 Hz, 1H), 7.35-7.29 (m, 2H), 7.24 (d, J=7.2 Hz, 1H), 7.20 (d, J=7.2 Hz, 2H), 7.08 (t, J=8.0 Hz, 1H), 7.03 (d, J=6.8 Hz, 1H), 6.58 (d, J=2.8 Hz, 1H), 5.43 (s, 2H), 4.76 (s, 2H), 3.61 (t, J=5.2 Hz, 2H), 2.92 (t, J=5.2 Hz, 2H), 2.42 (s, 3H); LCMS (ESI) m/z 295.2 [C₁₉H₂₂N₂O+H]⁺.

### Example 12: Synthesis of Compound 6 Via Synthesis Route E

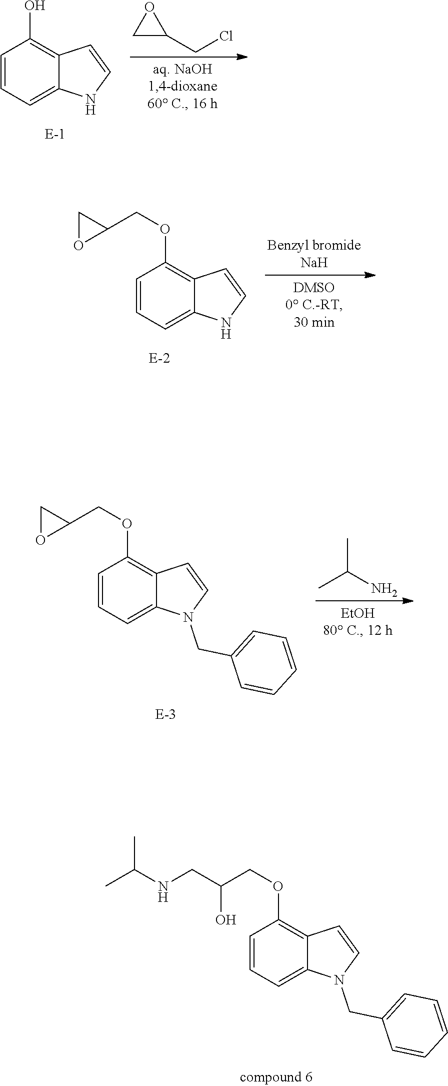

### Synthesis of 4-(oxiran-2-ylmethoxy)-1H-indole (E-2)

To a stirred solution of 1H-indol-4-ol E-1 (200 mg, 1.50 mmol, 1.0 equiv.) in 1,4-dioxane (2 mL) at 0° C., was added a solution of NaOH (72 mg, 1.80 mmol, 1.2 equiv.) in water (1 mL). The reaction mixture was stirred at RT for 20 min before epichlorohydrin (235 μL, 3.00 mmol, 2.0 equiv.) was added. The resultant mixture was stirred at 60° C. for 16 h. Upon the completion of the reaction by TLC, the reaction mixture was quenched with ice-cold water (15 mL) and extracted with EtOAc (2×30 mL). The combined organic layers were washed with brine, dried over Na₂SO₄, filtered, and concentrated under reduced pressure to afford the crude compound. The crude compound was purified by silica gel (60-120 mesh) column chromatography (elution with 15-25% EtOAc in hexane) to afford E-2 as a gummy solid (160 mg, yield: 56%). TLC system: EtOAc/hexane (30:70), R_{f} value=0.6; ¹H NMR (400 MHz, CDCl₃) δ ppm: 8.17 (br. s, 1H), 7.14-7.12 (m, 1H), 7.10-7.04 (m, 2H), 6.70-6.69 (m, 1H), 6.52 (d, J=7.6 Hz, 1H), 4.35 (dd, J=11.2 Hz, 3.6 Hz, 1H), 4.16 (dd, J=11.2 Hz, 5.6 Hz, 1H), 3.49-3.44 (m, 1H), 2.94 (t, J=4.8 Hz, 1H), 2.82 (dd, J=5.2 Hz, 2.8 Hz, 1H); LCMS (ESI) m/z 190.2 [C₁₁H₁₁NO₂+H]⁺.

### Synthesis of 1-benzyl-4-(oxiran-2-ylmethoxy)-1H-indole (E-3)

To a stirred solution of E-2 (160 mg, 0.85 mmol, 1.0 equiv.) in DMSO (1.6 mL) at 0° C., was added NaH (60% dispersion in mineral oil) (51 mg, 1.28 mmol, 1.5 equiv.). The reaction mixture was stirred at 0° C. for 20 min before benzyl bromide (0.12 mL, 1.02 mmol, 1.2 equiv.) was added. The resultant mixture was warmed to RT and stirred at RT for 30 min. Upon the completion of reaction by TLC, the reaction mixture was quenched with ice-cold water (15 mL) and extracted with EtOAc (2×30 mL). The combined organic layers were washed with brine, dried over Na₂SO₄, filtered, and concentrated under reduced pressure to afford the crude compound. The crude compound was purified by silica gel (60-120 mesh) column chromatography (elution with 5-10% EtOAc in hexane) to afford E-3 as an off-white gummy solid (180 mg, yield: 76%). TLC system: EtOAc/hexane (30:70), R_{f} value=0.4; ¹H NMR (400 MHz, CDCl₃) δ ppm: 7.31-7.28 (m, 1H), 7.24-7.23 (m, 1H), 7.09-7.04 (m, 4H), 6.93 (d, J=8.4 Hz, 1H), 6.68 (d, J=3.2 Hz, 1H), 6.52 (d, J=7.6 Hz, 1H), 5.31 (s, 2H), 4.35 (dd, J=11.2 Hz, 3.6 Hz, 1H), 4.16 (dd, J=11.2 Hz, 5.6 Hz, 1H), 3.47-4.43 (m, 1H), 2.93 (t, J=4.8 Hz, 1H), 2.82 (dd, J=4.8 Hz, 2.8 Hz, 1H); LCMS (ESI) m/z 280.1 [C₁₈H₁₇NO₂+H]⁺.

### Synthesis of 1-((1-benzyl-1H-indol-4-yl)oxy)-3-(isopropylamino)propan-2-ol as formic acid salt (Compound 6)

To a stirred solution of E-3 (180 mg, 0.64 mmol, 1.0 equiv.) in EtOH (1.8 mL) at RT, was added isopropylamine (0.28 mL, 3.22 mmol, 5.0 equiv.). The reaction mixture was stirred at 80° C. for 12 h. Upon the completion of reaction by TLC, the reaction mixture was concentrated under reduced pressure and purified by reverse phase column chromatography (Grace) (elution with 15-25% ACN in 0.1% FA in H₂O) to afford compound 6 (formic acid salt) as an off-white semi solid (116 mg, yield: 47%). TLC system: MeOH/CH₂Cl₂ (10:90), R_{f} value=0.2; ¹H NMR (400 MHz, DMSO-d₆) δ ppm: 8.31 (s, 1H; HCOOH), 7.38 (d, J=3.2 Hz, 1H), 7.31-7.27 (m, 2H), 7.25-7.21 (m, 1H), 7.16-7.14 (m, 2H), 7.04-6.97 (m, 2H), 6.52-6.51 (m, 2H), 5.39 (s, 2H), 4.07-4.04 (m, 3H), 3.04-2.93 (m, 2H), 2.80 (dd, J=12.0 Hz, 7.2 Hz, 1H), 1.10 (d, J=5.6 Hz, 6H); LCMS (ESI) m/z 339.3 [C₂₁H₂₆N₂O₂+H]⁺.

### Example 13: Synthesis of Compound 40 Via Synthesis Route F

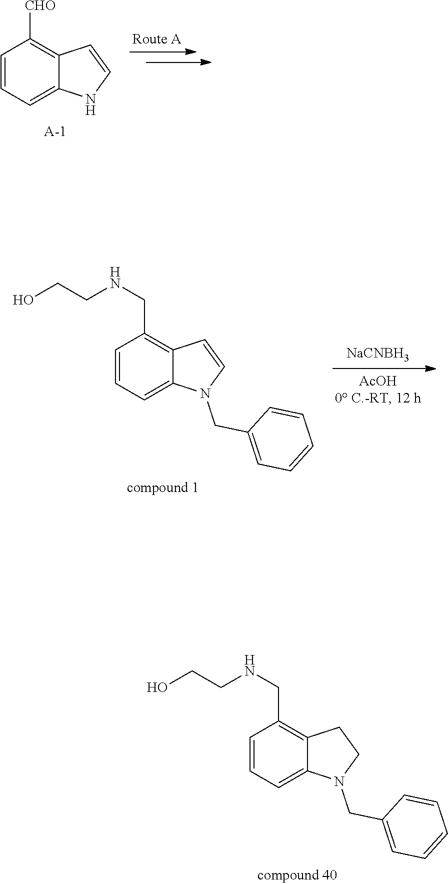

### Synthesis of 2-(((1-benzylindolin-4-yl)methyl)amino)ethan-1-ol as formic acid salt (Compound 40)

To a stirred solution of compound 1 (150 mg, 0.54 mmol, 1.0 equiv.) in AcOH (4.5 mL) at 0° C., was added NaBH₃CN (63 mg, 1.00 mmol, 1.87 equiv.). The resultant mixture was stirred at RT for 12 h. Upon the completion of reaction by TLC, the reaction mixture was quenched with aq. NaHCO₃ solution (10 mL) and extracted with 10% MeOH in CH₂Cl₂ (2×50 mL). The combined organic layers were dried over Na₂SO₄ and concentrated under reduced pressure. The crude obtained was purified by reverse phase column chromatography (Grace) (elution with 27% ACN in 0.01% FA in H₂O) to afford compound 40 (formic acid salt) as an off-white solid (62 mg, yield: 35%). TLC system: MeOH/CH₂Cl₂ (10:90), R_{f} value=0.15; melting point range: 98-102° C.; ¹H NMR (400 MHz, DMSO-d₆) δ ppm: 8.26 (s, 1H; HCOOH), 7.35-7.34 (m, 4H), 7.28-7.25 (m, 1H), 6.97 (t, J=7.6 Hz, 1H), 6.64 (d, J=7.6 Hz, 1H), 6.51 (d, J=8.0 Hz, 1H), 4.26 (s, 2H), 3.72 (s, 2H), 3.53 (t, J=5.6 Hz, 2H), 3.27 (t, J=8.4 Hz, 2H), 2.92 (t, J=8.4 Hz, 2H), 2.71 (t, J=5.6 Hz, 2H); LCMS (ESI) m/z 283.2 [C₁₈H₂₂N₂O+H]⁺.

### Example 14: Synthesis of Compound 42 Via Synthesis Route G

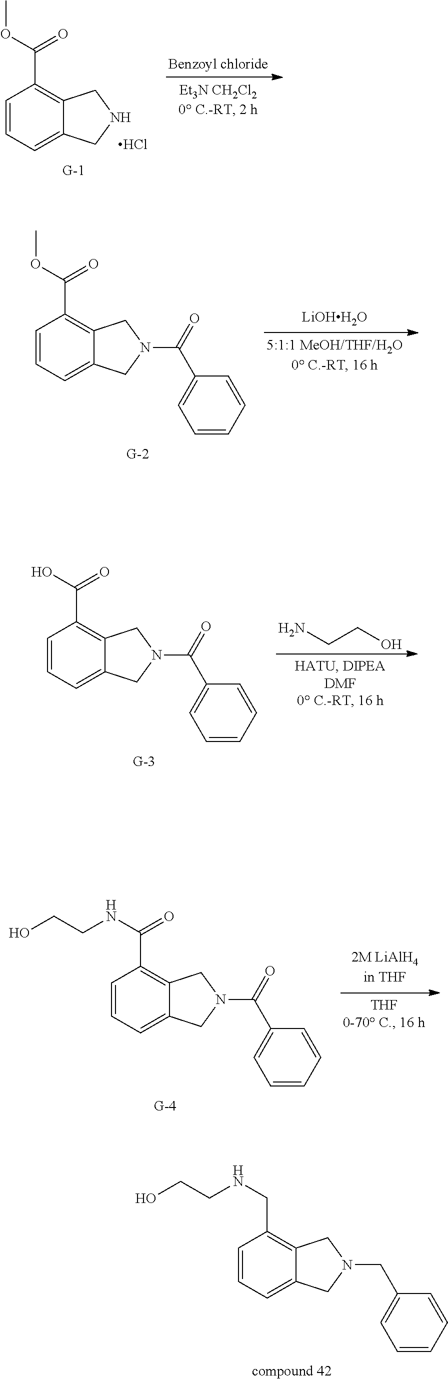

### Synthesis of methyl 2-benzoylisoindoline-4-carboxylate (G-2)

To a stirred solution of G-1 (500 mg, 2.34 mmol, 1.0 equiv.) in CH₂Cl₂ (5 mL) at 0° C., was added Et₃N (0.82 mL, 5.85 mmol, 2.5 equiv.) and benzoyl chloride (0.33 mL, 2.81 mmol, 1.2 equiv.). The resultant mixture was warmed to RT and stirred at RT for 2 h. Upon the completion of reaction by TLC, the reaction mixture was diluted with water (15 mL) and extracted with CH₂Cl₂ (2×20 mL). The combined organic layers were washed with brine, dried over Na₂SO₄, concentrated, and purified by silica gel (100-200 mesh) column chromatography (elution with 15% EtOAc in hexane) to afford G-2 (500 mg, yield: 76%) as an off-white solid. TLC system: EtOAc/hexane (30:70), R_{f} value=0.2; LCMS (ESI) m/z 282.1 [C₁₇H₁₅NO₃+H]⁺.

### Synthesis of 2-benzoylisoindoline-4-carboxylic acid (G-3)

To a stirred solution of G-2 (500 mg, 1.78 mmol, 1.0 equiv.) in 5:1:1 MeOH/THF/H₂O (7 mL) at 0° C., was added LiOH·H₂O (224 mg, 5.34 mmol, 3.0 equiv.). The resultant mixture was warmed to RT and stirred at RT for 16 h. Upon the completion of reaction by TLC, the volatiles were removed and the reaction mixture was acidified with 2N HCl (5 mL) and extracted with 10% MeOH in CH₂Cl₂ (2×20 mL). The combined organic layers were washed with brine, dried over Na₂SO₄, and concentrated to afford G-3 as an off-white solid (350 mg, yield: 74%). TLC system: MeOH/CH₂Cl₂ (5:95), R_{f} value=0.2; ¹H NMR (400 MHz, CDCl₃) δ ppm: 8.06-7.99 (m, 1H), 7.61-7.57 (m, 3H), 7.47-7.34 (m, 4H), 5.36-5.11 (d, 2H), 5.06-4.79 (d, 2H); LCMS (ESI) m/z 268.0 [C₁₆H₁₃NO₃+H]⁺.

### Synthesis of 2-benzoyl-N-(2-hydroxyethyl)isoindoline-4-carboxamide (G-4)

Compound G-4 was obtained following the amide coupling procedure similar to that for the synthesis of compound 4, utilizing 2-aminoethan-1-ol and reaction conditions of 0° C. to RT for 16 h. G-4 was obtained in 61% yield as a gummy liquid following reverse phase column chromatography (Grace) (elution with 25-30% ACN in 0.1% FA in H₂O). TLC system: MeOH/CH₂Cl₂ (10:90), R_{f} value=0.3; LCMS (ESI) m/z 311.1 [C₁₈H₁₈N₂O₃+H]⁺.

### Synthesis of 2-(((2-benzylisoindolin-4-yl)methyl)amino)ethan-1-ol (Compound 42)

To a stirred solution of G-4 (300 mg, 0.97 mmol, 1.0 equiv.) in THE (6 mL) at 0° C., was added 2 M LiAlH₄ in THE solution (2.43 mL, 4.85 mmol, 5.0 equiv.). The resultant mixture was warmed and stirred at 70° C. for 16 h. Upon the completion of reaction by TLC, the reaction mixture was quenched with the addition of aq. NH₄Cl (10 mL) and extracted with 10% MeOH in CH₂Cl₂ (2×20 mL). The combined organic layers were dried over Na₂SO₄, concentrated under reduced pressure, and purified by Prep-HPLC (ammonium bicarbonate buffer) to afford compound 42 as a light brown gummy liquid (48 mg, yield: 18%). TLC system: MeOH/CH₂Cl₂ (10:90), R_{f} value=0.1; ¹H NMR (400 MHz, DMSO-d₆) δ ppm: 7.40-7.33 (m, 4H), 7.29-7.25 (m, 1H), 7.16-7.13 (m, 2H), 7.09-7.07 (m, 1H), 4.44 (t, J=5.2 Hz, 1H), 3.88 (s, 2H), 3.87 (s, 2H), 3.83 (s, 2H), 3.61 (s, 2H), 3.43 (q, J=5.2 Hz, 2H), 2.53 (t, J=6.0 Hz, 2H); LCMS (ESI) m/z 283.1 [C₁₈H₂₂N₂O+H]⁺.

### Example 15: Synthesis of Compound 52 Via Synthesis Route H

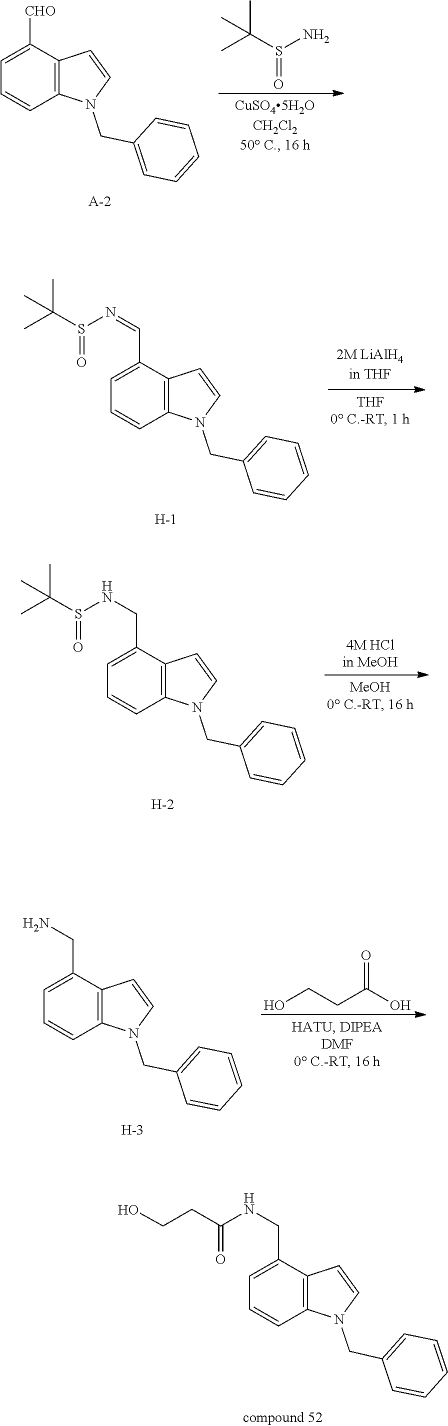

### Synthesis of (EZ)—N-((1-benzyl-1H-indol-4-yl)methylene)-2-methylpropane-2-sulfinamide (H-1)

To a stirred solution of 1-benzyl-1H-indole-4-carbaldehyde A-2 (2.00 g, 8.50 mmol, 1.0 equiv.) in CH₂Cl₂ (20 mL) at RT, was added 2-methylpropane-2-sulfinamide (3.61 g, 29.8 mmol, 3.5 equiv.) and CuSO₄·5CH₂O (3.20 g, 12.8 mmol, 1.5 equiv.). The resultant mixture was stirred at 50° C. for 16 h. Upon the completion of reaction by TLC, the reaction mixture was diluted with water and extracted with CH₂Cl₂ (3×70 mL). The combined organic layers were dried over Na₂SO₄, filtered, and concentrated under reduced pressure. The crude obtained was purified by silica gel (60-120 mesh) column chromatography (elution with 20% EtOAc in hexane) to afford H-1 (1.5 g, yield: 52%) as pale yellow solid. TLC system: EtOAc/hexane (20:80), R_{f} value=0.3; LCMS (ESI) m/z 339.1 [C₂₀H₂₂N₂OS+H]⁺.

### Synthesis of N-((1-benzyl-1H-indol-4-yl)methyl)-2-methylpropane-2-sulfinamide (H-2)

To a stirred solution of H-1 (1.50 g, 4.43 mmol, 1.0 equiv.) in THE (30 mL) at 0° C., was added 2 M LiAlH₄ in THE solution (4.43 mL, 8.86 mmol, 2.0 equiv.). The resultant mixture was warmed to RT and stirred at RT for 1 h. Upon the completion of reaction by TLC, the reaction mixture was quenched with sat. NH₄Cl solution (50 mL) and extracted with CH₂Cl₂ (2×100 mL). The combined organic layers were dried over Na₂SO₄, filtered, and concentrated under reduced pressure. The crude obtained was purified by silica gel (100-200 mesh) column chromatography (elution with 30% EtOAc in hexane) to afford H-2 (1.4 g, yield: 93%) as yellow gummy liquid. TLC system: EtOAc/hexane (30:70), R_{f} value=0.2; ¹H NMR (400 MHz, CDCl₃) δ ppm: 7.32-7.27 (m, 3H), 7.26-7.24 (m, 1H), 7.16-7.09 (m, 5H), 6.63 (d, J=2.8 Hz, 1H), 5.33 (s, 2H), 4.67 (dd, J=13.2 Hz, 4.0 Hz, 1H), 4.53 (dd, J=13.2 Hz, 4.0 Hz, 1H), 3.54-3.51 (m, 1H), 1.25 (s, 9H); LCMS (ESI) m/z 341.2 [C₂₀H₂₄N₂OS+H]⁺.

### Synthesis of (1-benzyl-1H-indol-4-yl)methanamine (H-3)

In a sealed tube, to a stirred solution of H-2 (1.4 g, 4.10 mmol, 1.0 equiv.) in MeOH (14 mL) at 0° C., was added 4M HCl in MeOH (2.8 mL). The resultant mixture was warmed to RT and stirred at RT for 16 h. Upon the completion of reaction, the volatiles were removed under vacuum and the reaction mixture was diluted with water (50 mL), neutralized with sat. NaHCO₃ solution and extracted with 10% MeOH in CH₂Cl₂ (3×50 mL). The combined organic layers were dried over Na₂SO₄, filtered, concentrated, and purified by silica gel (100-200 mesh) column chromatography (elution with 50% EtOAc in hexane) to afford H-3 (900 mg, yield: 93%) as yellow colour gummy. TLC system: EtOAc/hexane (50:50), R_{f} value=0.1; ¹H NMR (400 MHz, CDCl₃) δ ppm: 7.31-7.25 (m, 3H), 7.20 (d, J=8.0 Hz, 1H), 7.17-7.06 (m, 5H), 6.62 (d, J=2.8 Hz, 1H), 5.33 (s, 2H), 4.17 (s, 2H); LCMS (ESI) m/z 237.2 [C₁₆H₁₆N₂+H]⁺.

### Synthesis of N-((1-benzyl-1H-indol-4-yl)methyl)-2-hydroxyacetamide (Compound 52)

To a stirred solution of 3-hydroxypropanoic acid (30% solution in water) (1.02 mL, 3.40 mmol, 4.0 equiv.) in DMF (3 mL) at 0° C., was added DIPEA (0.3 mL, 1.70 mmol, 2.0 equiv.), HATU (422 mg, 1.11 mmol, 1.3 equiv.) and H-3 (200 mg, 0.85 mmol, 1.0 equiv.). The reaction mixture was warmed to RT and stirred at RT for 16 h. Upon the completion of reaction by TLC, the reaction mixture was diluted with ice-cold water and extracted with 10% MeOH in CH₂Cl₂ (3×50 mL). The combined organic layers were dried over Na₂SO₄, filtered, concentrated under reduced pressure, and purified by reverse phase column chromatography (Grace) (elution with 30% ACN in 0.1% FA in H₂O) to afford compound 52 (45 mg, yield: 17%) as off-white solid. TLC system: MeOH/CH₂Cl₂ (10:90), R_{f} value=0.6; melting point range: 110-113° C.; ¹H NMR (400 MHz, DMSO-d₆) δ ppm: 8.24 (t, J=5.6 Hz, 1H), 7.49 (d, J=3.2 Hz, 1H), 7.35-7.27 (m, 3H), 7.24-7.21 (m, 1H), 7.19-7.17 (m, 2H), 7.03 (t, J=8.0 Hz, 1H), 6.91 (d, J=7.2 Hz, 1H), 6.55 (d, J=2.8 Hz, 1H), 5.42 (s, 2H), 4.57 (t, J=5.2 Hz, 1H), 4.51 (d, J=5.6 Hz, 2H), 3.66-3.62 (m, 2H), 2.30 (t, J=6.4 Hz, 2H); LCMS (ESI) m/z 309.1 [C₁₉H₂₀N₂O₂+H]⁺.

### Example 16: Synthesis of Compound 53 Via Synthesis Route H

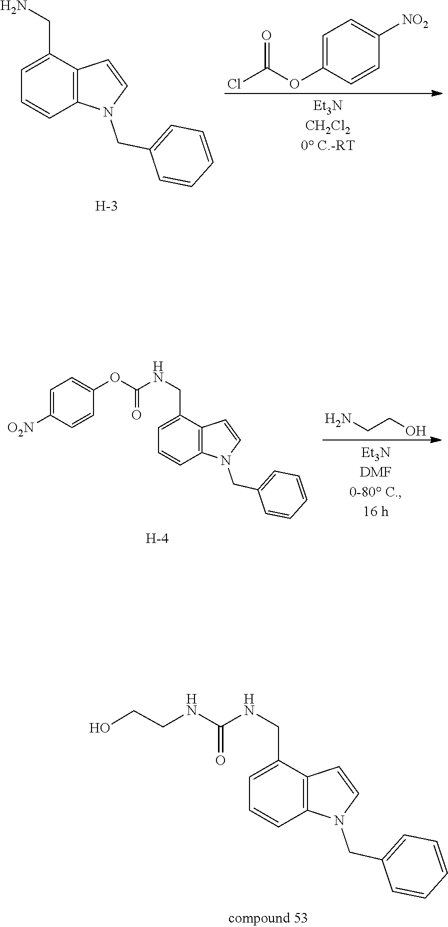

### Synthesis of 4-nitrophenyl ((1-benzyl-1H-indol-4-yl)methyl)carbamate (H-4)

In a sealed tube, to a stirred solution of H-3 (200 mg, 0.85 mmol, 1.0 equiv.) in CH₂Cl₂ (4 mL) at 0° C., was added Et₃N (0.3 mL, 2.13 mmol, 2.5 equiv.) and 4-nitrophenyl carbonochloridate (206 mg, 1.02 mmol, 1.2 equiv.). The resultant mixture was warmed to RT and stirred at RT for 16 h. Upon the completion of reaction by TLC, the reaction mixture was diluted with ice-cold water (50 mL) and extracted with CH₂Cl₂ (2×50 mL). The combined organic layers were dried over Na₂SO₄, filtered, and concentrated under reduced pressure. The crude obtained was washed with n-pentane to afford H-4 (300 mg) as a pale-yellow liquid in its crude. TLC system: MeOH/CH₂Cl₂ (5:95), R_{f} value=0.7; LCMS (ESI) m/z 402.2 [C₂₃H₁₉N₃O₄+H]⁺. The crude compound was used directly in the next step without further purification.

### Synthesis of 1-((1-benzyl-1H-indol-4-yl)methyl)-3-(2-hydroxyethyl)urea (Compound 53)

To a stirred solution of H-4 (300 mg, 0.75 mmol, 1.0 equiv.) in DMF (3 mL) at 0° C., was added Et₃N (0.2 mL, 1.50 mmol, 2.0 equiv.) and 2-aminoethan-1-ol (91 μL, 1.50 mmol, 2.0 equiv.). The resultant mixture was stirred at 80° C. for 16 h. Upon the completion of reaction by TLC, the reaction mixture was diluted with ice-cold water and extracted with 10% MeOH in CH₂Cl₂ (3×30 mL). The combined organic layers were dried over Na₂SO₄, filtered, concentrated under reduced pressure, and purified by reverse phase column chromatography (Grace) (elution with 28% ACN in 0.1% FA in H₂O) to afford compound 53 (95 mg, yield: 35% over 2 steps) as an off-white solid. TLC system: MeOH/CH₂Cl₂ (5:95), R_{f} value=0.2; ¹H NMR (400 MHz, DMSO-d₆) δ ppm: 7.49 (d, J=7.2 Hz, 1H), 7.33-7.27 (m, 3H), 7.24-7.21 (m, 1H), 7.18-7.16 (m, 2H), 7.03 (t, J=8.0 Hz, 1H), 6.89 (d, J=7.2 Hz, 1H), 6.56 (d, J=3.2 Hz, 1H), 6.36 (d, J=5.6 Hz, 1H), 5.93 (t, J=5.6 Hz, 1H), 5.41 (s, 2H), 4.65 (t, J=5.2 Hz, 1H), 4.45 (d, J=6.0 Hz, 2H), 3.40-3.36 (m, 2H), 3.11-3.07 (m, 2H); LCMS (ESI) m/z 324.2 [C₁₉H₂₁N₃O₂+H]⁺.

### Example 17: Synthesis of Compound 83 Via Synthesis Route I

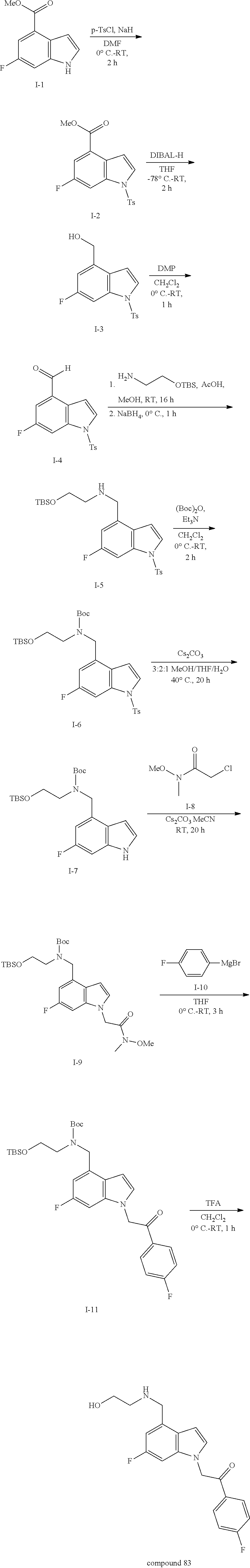

### Synthesis of methyl 6-fluoro-1-tosyl-1H-indole-4-carboxylate (I-2)

To a stirred solution of methyl 6-fluoro-1H-indole-4-carboxylate I-1 (7.50 g, 38.8 mmol, 1.0 equiv.) in DMF (150 mL) at 0° C., was added NaH (60% dispersion in mineral oil) (3.10 g, 77.6 mmol, 2.0 equiv.) in portions under nitrogen atmosphere. The mixture was stirred at 0° C. for 30 min before p-TsCl (11.1 g, 58.2 mmol, 1.5 equiv.) was added in portions. The resultant mixture was warmed to RT and stirred at RT for 2 h. Upon the completion of the reaction by TLC, the reaction mixture was quenched with saturated aq. NH₄Cl (100 mL) and extracted with EtOAc (2×100 mL). The combined organic layers were washed with ice-cold water (300 mL), dried over Na₂SO₄, filtered and concentrated under reduced pressure. The obtained crude material was taken up in MeOH (50 mL), stirred for 15 min, filtered and dried to afford I-2 (6.00 g, yield: 45%) as an off-white solid. TLC system: EtOAc/hexane (20:80), R_{f} value=0.5; ¹H NMR (400 MHz, CDCl₃) δ ppm: 7.96-7.93 (m, 1H), 7.75 (d, J=8.4 Hz, 2H), 7.70 (dd, J=9.6 Hz, 2.4 Hz, 1H), 7.67 (d, J=4.0 Hz, 1H), 7.32 (d, J=4.0 Hz, 1H), 7.24 (2H, overlapped with solvent signal), 3.95 (s, 3H), 2.36 (s, 3H); LCMS (ESI) m/z 347.9 [C₁₇H₁₄FNO₄S+H]⁺.

### Synthesis of (6-fluoro-1-tosyl-1H-indol-4-yl)methanol (I-3)

To a stirred solution of I-2 (6.00 g, 17.3 mmol, 1.0 equiv.) in THE (60 mL) at −78° C., was added DIBAL-H (1 M in hexane) (51.9 mL, 51.9 mmol, 3.0 equiv.) under nitrogen atmosphere. The resultant mixture was warmed to RT and stirred at RT for 2 h. Upon the completion of the reaction by TLC, the reaction mixture was quenched with saturated aq. NH₄Cl solution. The precipitate was filtered, washing with EtOAc. The filtrate was extracted with EtOAc (2×100 mL), dried over Na₂SO₄, filtered and concentrated under reduced pressure. The crude material obtained was purified by silica gel (230-400 mesh) flash column chromatography (gradient elution with 0-10% EtOAc in hexane) to afford I-3 (4.50 g, yield: 81%) as an off-white solid. TLC system: EtOAc/hexane (20:80), R_{f} value=0.4; ¹H NMR (400 MHz, DMSO-d₆) δ ppm: 7.90 (d, J=8.4 Hz, 2H), 7.81 (d, J=3.6 Hz, 1H), 7.59 (dd, J=9.6 Hz, 1.6 Hz, 1H), 7.39 (d, J=8.0 Hz, 2H), 7.11 (dd, J=10.0 Hz, 2.0 Hz, 1H), 6.92 (d, J=4.4 Hz, 1H), 5.40 (t, J=5.6 Hz, 1H), 4.70 (d, J=6.0 Hz, 2H), 2.32 (s, 3H); LCMS (ESI) m/z 319.9 [C₁₆H₁₄FNO₃S+H]⁺.

### Synthesis of 6-fluoro-1-tosyl-1H-indole-4-carbaldehyde (I-4)

To a stirred solution of I-3 (8.49 g, 26.6 mmol, 1 equiv.) in CH₂Cl₂ (135 mL) at 0° C., was added DMP (22.6 g, 53.2 mmol, 2.0 equiv.) in portions under nitrogen atmosphere. The resultant mixture was warmed to RT and stirred at RT for 1 h. Upon the completion of the reaction by TLC, the reaction mixture was filtered through a pad of celite. The filtrate was washed with saturated aq. NaHCO₃ (2×75 mL) and the aqueous layer was back extracted with CH₂Cl₂ (50 mL). The combined organic layers were dried over anhydrous Na₂SO₄, filtered and concentrated under reduced pressure. The crude material obtained was purified by silica gel (100-200 mesh) column chromatography (gradient elution with 0-10% EtOAc in hexanes) to afford I-4 (7.51 g, yield: 89%) as an off-white solid. TLC system: EtOAc/hexanes (20:80), R_{f} value=0.35; ¹H NMR (400 MHz, CDCl₃) δ ppm: 10.13 (s, 1H), 8.03-7.99 (m, 1H), 7.77 (d, J=8.8 Hz, 2H), 7.74 (d, J=3.6 Hz, 1H), 7.48 (dd, J=8.8 Hz, 2.4 Hz, 1H), 7.42 (d, J=4.4 Hz, 1H), 7.28 (2H, overlapped with solvent signal), 2.37 (s, 3H); LCMS (ESI) m/z 317.9 [C₁₆H₁₂FNO₃S+H]⁺.

### Synthesis of 2-((tert-butyldimethylsilyl)oxy)-N-((6-fluoro-1-tosyl-1H-indol-4-yl)methyl)ethan-1-amine (I-5)

To a stirred solution of I-4 (4.20 g, 13.2 mmol, 1.0 equiv.) and 2-((tert-butyldimethylsilyl)oxy)ethan-1-amine (2.31 g, 13.2 mmol, 1.0 equiv.) in MeOH (42 mL) at RT, was added AcOH (0.38 mL, 6.60 mmol, 0.5 equiv.). The reaction mixture was stirred at 70° C. for 2 h before it was cooled to 0° C., and NaBH₄ (999 mg, 26.4 mmol, 2.0 equiv.) was added. The resultant mixture was warmed to RT and stirred at RT for 2 h. Upon the completion of reaction by TLC, the reaction mixture was quenched with ice-cold water (30 mL) and extracted with EtOAc (2×70 mL). The combined organic layers were dried over Na₂SO₄, filtered and concentrated under reduced pressure. The crude material obtained was purified by silica gel (230-400 mesh) column chromatography (gradient elution with 0-30% EtOAc in hexanes) to afford I-5 (3.50 g, yield: 56%) as a pale-yellow gum. TLC system: EtOAc/hexane (40:60), R_{f} value=0.3; ¹H NMR (400 MHz, DMSO-d₆) δ ppm: 7.94 (d, J=8.4 Hz, 2H), 7.82 (d, J=3.6 Hz, 1H), 7.61 (dd, J=9.6 Hz, 2.0 Hz, 1H), 7.43 (d, J=8.0 Hz, 2H), 7.17 (dd, J=10.4 Hz, 2.4 Hz, 1H), 6.99 (d, J=3.6 Hz, 1H), 3.95 (s, 2H), 3.64 (t, J=6.0 Hz, 2H), 2.60 (t, J=6.0 Hz, 2H), 2.37 (s, 3H), 2.18 (br. s, 1H), 0.83 (s, 9H), −0.00 (s, 6H); LCMS (ESI) m/z 477.4 [C₂₄H₃₃FN₂O₃SSi+H]⁺.

### Synthesis of tert-butyl (2-((tert-butyldimethylsilyl)oxy)ethyl)((6-fluoro-1-tosyl-1H-indol-4-yl)methyl)carbamate (I-6)

To a stirred solution of I-5 (3.50 g, 7.34 mmol, 1.0 equiv.) in CH₂Cl₂ (35 mL) at 0° C., were added Et₃N (2.05 mL, 14.7 mmol, 2.0 equiv.) and (Boc)₂O (2.53 mL, 11.0 mmol, 1.5 equiv.). The resultant mixture was warmed to RT and stirred at RT for 2 h. Upon the completion of the reaction by TLC, the reaction mixture was diluted with water (100 mL) and extracted with CH₂Cl₂ (2×75 mL). The combined organic layers were dried over Na₂SO₄, filtered and concentrated under reduced pressure. The crude material obtained was purified by silica gel (230-400 mesh) column chromatography (gradient elution with 0-20% EtOAc in hexane) to afford I-6 (4.00 g, yield: 94%) as a pale-yellow gummy. TLC system: EtOAc/hexane (20:80), R_{f} value=0.5; ¹H NMR (400 MHz, DMSO-d₆) δ ppm: 7.93-7.88 (m, 2H), 7.84 (d, J=3.6 Hz, 1H), 7.62 (dd, J=9.6 Hz, 2.0 Hz, 1H), 7.39 (d, J=8.0 Hz, 2H), 6.95 (dd, J=10.4 Hz, 2.0 Hz, 1H), 6.84 (s, 1H), 4.62 (s, 2H), 3.57-3.50 (m, 2H), 3.29-3.16 (m, 2H), 2.32 (s, 3H), 1.41 (s, 6H) and 1.18 (s, 3H) tert-butyl protons of Boc split into two peaks, 0.76 (s, 9H), −0.09 (s, 6H); LCMS (ESI) m/z 577.4 [C₂₉H₄₁FN₂O₅SSi+H]⁺.

### Synthesis of tert-butyl (2-((tert-butyldimethylsilyl)oxy)ethyl)((6-fluoro-1H-indol-4-yl)methyl)carbamate (I-7)

To a stirred solution of I-6 (500 mg, 0.867 mmol, 1.0 equiv.) in a 3:2:1 mixture of MeOH/THF/H₂O (5 mL) was added Cs₂CO₃ (847 mg, 2.60 mmol, 3.0 equiv.) at RT. The resultant reaction mixture was stirred at 40° C. for 20 h, before it was cooled to RT, diluted with water (30 mL), and extracted with EtOAc (2×25 mL). The combined organic layers were dried over anhydrous Na₂SO₄, filtered and concentrated under reduced pressure. The crude material obtained was purified by silica gel (230-400 mesh) column chromatography (gradient elution with 0-20% EtOAc in hexane) to afford I-7 (100 mg, yield: 27%) as an off-white gum. TLC system: EtOAc/hexane (20:80), R_{f} value=0.4; ¹H NMR (400 MHz, DMSO-d₆) δ ppm: 11.22 (s, 1H), 7.34 (s, 1H), 7.08 (d, J=10.0 Hz, 1H), 6.69-6.66 (m, 1H), 6.43 (s, 1H), 4.69 (s, 2H), 3.62-3.55 (m, 2H), 3.26-3.20 (m, 1H), 3.17-3.14 (m, 1H), 1.45-1.35 (d, 9H), 0.85 (s, 9H), −0.00 (s, 6H); LCMS (ESI) m/z 423.1 [C₂₂H₃₅FN₂O₃Si+H]⁺.

### Synthesis of tert-butyl (2-((tert-butyldimethylsilyl)oxy)ethyl)((6-fluoro-1-(2-(methoxy(methyl)amino)-2-oxoethyl)-1H-indol-4-yl)methyl)carbamate (I-9)

To a stirred solution of I-7 (600 mg, 1.42 mmol, 1.0 equiv.) in MeCN (6 mL) was added Cs₂CO₃ (925 mg, 2.84 mmol, 2.0 equiv.) at RT. The reaction mixture was stirred for 10 minutes before it was cooled to 0° C., and 2-chloro-N-methoxy-N-methylacetamide I-8 (391 mg, 2.84 mmol, 2.0 equiv.) was added. The resultant mixture was warmed to RT and stirred at RT for 20 h. Upon the completion of the reaction by TLC, the reaction mixture was diluted with H₂O (50 mL) and extracted with EtOAc (2×50 mL). The combined organic layers were dried over anhydrous Na₂SO₄, filtered and concentrated under reduced pressure. The crude material obtained was purified by silica gel (230-400 mesh) column chromatography (gradient elution with 0-30% EtOAc in hexanes) to afford I-9 (400 mg, yield: 54%) as a pale-yellow gum. TLC system: EtOAc/hexane (30:70), R_{f} value=0.5; ¹H NMR (400 MHz, DMSO-d₆) δ ppm: 7.32-7.28 (m, 1H), 7.18-7.15 (m, 1H), 6.71-6.64 (m, 1H), 6.45-6.42 (m, 1H), 5.16 (s, 2H), 4.68 (s, 2H), 3.81 (s, 3H), 3.62-3.58 (m, 2H), 3.23-3.13 (m, 2H), 3.12 (s, 3H), 1.43-1.34 (d, 9H), 0.84 (s, 9H), −0.00 (s, 6H); LCMS (ESI) m/z 424.3 [C₂₆H₄₂FN₃O₅Si-Boc+H]⁺.

### Synthesis of tert-butyl (2-((tert-butyldimethylsilyl)oxy)ethyl)((6-fluoro-1-(2-(4-fluorophenyl)-2-oxoethyl)-1H-indol-4-yl)methyl)carbamate (I-11)

To a stirred solution of I-9 (125 mg, 0.239 mmol, 1.0 equiv.) in THE (2 mL) at 0° C., was added (4-fluorophenyl)magnesium bromide I-10 (1 M in THF, 0.478 mL, 0.478 mmol, 2.0 equiv.) drop-wise under nitrogen atmosphere. The resultant mixture was warmed to RT and stirred at RT for 3 h. Upon the completion of the reaction by TLC, the reaction mixture was cooled to 0° C., quenched with saturated aq. NH₄Cl (20 mL) and extracted with EtOAc (2×50 mL). The combined organic layers were dried over Na₂SO₄, filtered and concentrated under reduced pressure. The crude material obtained was purified by silica gel (230-400 mesh) flash column chromatography (gradient elution with 0-15% EtOAc in hexanes) to afford I-11 (62 mg, yield: 46%) as pale-yellow gum. TLC system: EtOAc/hexane (20:80), R_{f} value=0.5; ¹H NMR (400 MHz, DMSO-d₆) δ ppm: 8.18-8.15 (m, 2H), 7.45 (t, J=8.8 Hz, 2H), 7.32 (s, 1H), 7.25 (d, J=10.0 Hz, 1H), 6.75-6.68 (m, 1H), 6.52-6.48 (m, 1H), 5.89 (s, 2H), 4.72 (s, 2H), 3.64-3.61 (m, 2H), 3.27-3.20 (m, 2H), 1.46-1.36 (d, 9H), 0.86 (s, 9H), 0.02 (s, 6H); LCMS (ESI) m/z 559.0 [C₃₀H₄₀F₂N₂O₄Si+H]⁺.

### Synthesis of 2-(6-fluoro-4-(((2-hydroxyethyl)amino)methyl)-1H-indol-1-yl)-1-(4-fluorophenyl)ethan-1-one (compound 83)

To a stirred solution of I-11 (250 mg, 0.447 mmol, 1.0 equiv.) in CH₂Cl₂ (2.5 mL) at 0° C., was added TFA (0.25 mL) under nitrogen atmosphere. The resultant mixture was warmed to RT and stirred at RT for 1 h. Upon the completion of the reaction by TLC, the reaction mixture was concentrated under reduced pressure. The obtained residue was diluted with 10% MeOH in CH₂Cl₂ (20 ml) and washed with sat. NaHCO₃ solution. The organic layer was dried over Na₂SO₄, filtered and concentrated under reduced pressure. The crude material obtained was purified by prep-HPLC [Prep conditions: X-Terra C18 column, 5 μm, 19×150 mm; Buffer A: 0.1% NH₃ in H₂O; Buffer B: MeCN/H₂O (80:20); Gradient (Time/% B): 0/20, 14/60, 14.1/98; Flow rate: 12 mL/min] to afford compound 83 (45.0 mg, yield: 29%) as an off-white solid. TLC system: MeOH/CH₂Cl₂ (10:90), R_{f} value=0.2; melting point range: 127.6-131.9° C.; ¹H NMR (400 MHz, DMSO-d₆) δ ppm: 8.19-8.15 (m, 2H), 7.47-7.42 (m, 2H), 7.29 (d, J=3.2 Hz, 1H), 7.16 (dd, J=10.0 Hz, 2.0 Hz, 1H), 6.91 (dd, J=10.8 Hz, 2.4 Hz, 1H), 6.58 (d, J=3.2 Hz, 1H), 5.87 (s, 2H), 4.50 (t, J=5.6 Hz, 1H), 3.98 (s, 2H), 3.50 (q, J=5.6 Hz, 2H), 2.64 (t, J=5.6 Hz, 2H); LCMS (ESI) m/z 345.3 [C₁₉H₁₈F₂N₂O₂+H]⁺.

### Example 18: Synthesis of Compound 110 Via Synthesis Route J

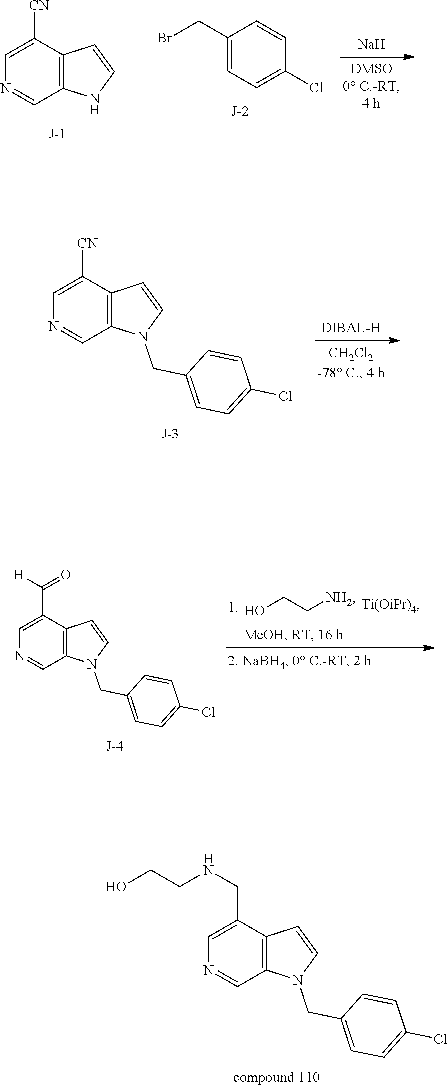

### Synthesis of 1-(4-chlorobenzyl)-1H-pyrrolo[2,3-c]pyridine-4-carbonitrile (J-3)

To a stirred solution of 1H-pyrrolo[2,3-c]pyridine-4-carbonitrile J-1 (1.00 g, 6.99 mmol, 1.0 equiv.) in DMSO (10 mL) at 0° C., was added NaH (60% dispersion in mineral oil) (700 mg, 17.5 mmol, 2.5 equiv.). The reaction mixture was allowed to stir at RT for 20 min, before it was cooled to 0° C., and J-2 (1.72 g, 8.39 mmol, 1.2 equiv.) was added. The resultant mixture was warmed to RT and stirred at RT for 4 h. Upon the completion of reaction by TLC, the reaction mixture was poured into ice-cold water (100 mL) and extracted with EtOAc (3×150 mL). The combined organic layers were washed with brine, dried over anhydrous Na₂SO₄, and concentrated under reduced pressure. The crude material obtained was purified by silica gel (100-200 mesh) column chromatography (gradient elution with 30-40% EtOAc in hexane) to afford J-3 (400 mg, yield: 21%) as a yellow solid. TLC system: EtOAc/hexane (40:60), R_{f} value=0.2; ¹H NMR (400 MHz, CDCl₃) δ ppm: 8.84 (s, 1H), 8.59 (s, 1H), 7.49 (d, J=3.2 Hz, 1H), 7.34-7.32 (m, 2H), 7.08 (d, J=8.4 Hz, 2H), 6.81 (dd, J=2.8 Hz, 0.8 Hz, 1H), 5.44 (s, 2H); LCMS (ESI) m/z 268.2 [C₁₅H₁₀ClN₃+H]⁺.

### Synthesis of 1-(4-chlorobenzyl)-1H-pyrrolo[2,3-c]pyridine-4-carbaldehyde (J-4)

To a stirred solution of J-3 (400 mg, 1.49 mmol, 1.0 equiv.) in anhydrous CH₂Cl₂ (4 mL) at −78° C., was added DIBAL-H (1.5 M in toluene) (1.99 mL, 2.98 mmol, 2.0 equiv.) under nitrogen atmosphere. The resultant mixture was stirred at −78° C. for 4 h. Upon the completion of reaction by TLC, the reaction mixture was quenched with aq. sat. NH₄Cl solution (10 mL) at −78° C., allowed to warm to RT, diluted with water (20 mL) and extracted with CH₂Cl₂ (3×30 mL). The combined organic layers were dried over anhydrous Na₂SO₄, filtered and concentrated under reduced pressure. The crude material obtained was purified by silica gel (100-200 mesh) column chromatography (gradient elution with 30-40% EtOAc in hexane) to afford J-4 (130 mg, yield: 32%) as a yellow solid. TLC system: EtOAc/hexane (40:60), R_{f} value=0.3; ¹H NMR (400 MHz, CDCl₃) δ ppm: 10.31 (s, 1H), 8.89 (s, 1H), 8.72 (s, 1H), 7.54 (d, J=2.8 Hz, 1H), 7.36-7.33 (m, 3H), 7.08 (d, J=8.0 Hz, 2H), 5.47 (s, 2H); LCMS (ESI) m/z 271.1 [C₁₅H₁₁ClN₂O+H]⁺.

### Synthesis of 2-(((1-(4-chlorobenzyl)-1H-pyrrolo[2,3-c]pyridin-4-yl)methyl)amino)ethan-1-ol (Compound 110)

To a stirred solution of J-4 (130 mg, 0.480 mmol, 1.0 equiv.) in MeOH (1.30 mL) at RT was added 2-aminoethan-1-ol (58.0 μL, 0.960 mmol, 2.0 equiv.), followed by Ti(OiPr)₄ (0.130 mL). The reaction mixture was stirred at RT for 16 h before it was cooled to 0° C., and NaBH₄ (36.3 mg, 0.960 mmol, 2.0 equiv.) was added portion-wise. The resultant reaction mixture was stirred at RT for 2 h. Upon the completion of reaction by TLC, the reaction mixture was quenched with ice-cold water (20 mL) and extracted with 10% MeOH in CH₂Cl₂ (2×25 mL). The combined organic layers were dried over anhydrous Na₂SO₄, concentrated under reduced pressure and purified by prep-HPLC [Prep conditions: Gemini C18 column, 5 μm, 21.2×250 mm; Buffer A: 0.1% liquid NH₃ in 10 mM ammonium bicarbonate in water; Buffer B: MeCN/H₂O (80:20); Gradient (Time/% B): 0/35, 16/75, 16.1/98; Flow Rate: 13 mL/min] to afford compound 110 as a white solid (28.0 mg, yield: 18%). TLC system: MeOH/CH₂Cl₂ (10:90), R_{f} value=0.1; melting point range: 49.3-54.1° C.; ¹H NMR (400 MHz, DMSO-d₆) δ ppm: 8.71 (s, 1H), 8.06 (s, 1H), 7.73 (d, J=3.2 Hz, 1H), 7.40-7.37 (m, 2H), 7.29-7.26 (m, 2H), 6.67 (dd, J=3.2 Hz, 0.8 Hz, 1H), 5.53 (s, 2H), 4.46 (t, J=5.6 Hz, 1H), 3.95 (s, 2H), 3.46 (q, J=5.6 Hz, 2H), 2.59 (t, J=5.6 Hz, 2H); LCMS (ESI) m/z 316.0 [C₁₇H₁₈ClN₃O+H]⁺.

### Example 19: Synthesis of Compound 84 Via Synthesis Route A

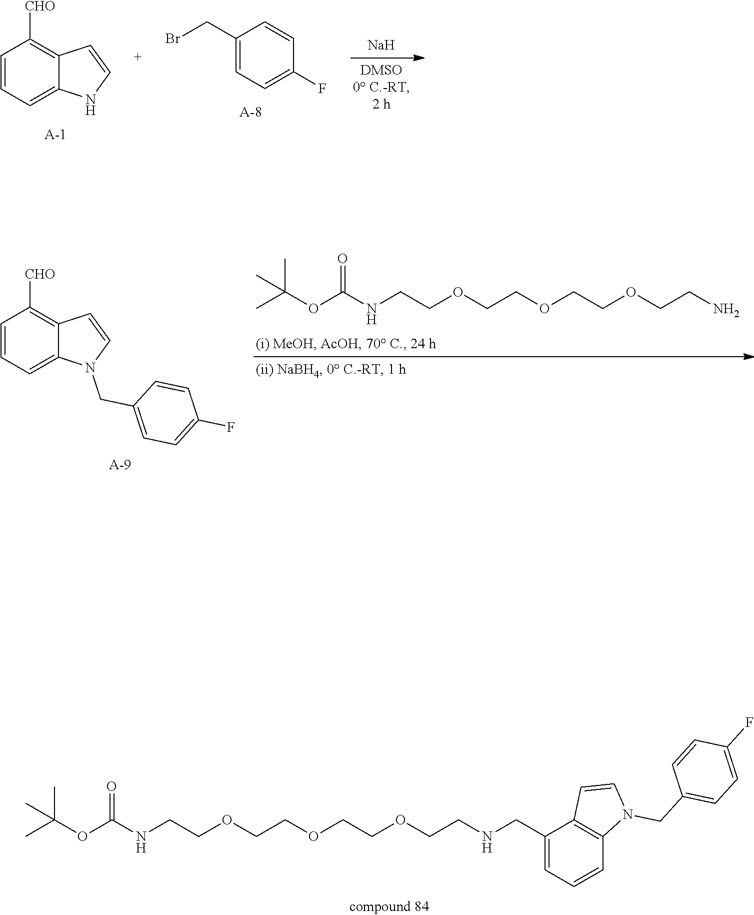 [SMILES: O=Cc1cccc2[nH]ccc12 ; Fc1ccc(CBr)cc1 ; O=Cc1cccc2c1ccn2Cc1ccc(F)cc1 ; CC(C)(C)OC(=O)NCCOCCOCCOCCN ; CC(C)(C)OC(=O)NCCOCCOCCOCCNCc1cccc2c1ccn2Cc1ccc(F)cc1]

### Synthesis of 1-(4-fluorobenzyl)-1H-indole-4-carbaldehyde (A-9)

Compound A-9 was obtained following the alkylation procedure similar to that for the synthesis of compound A-2, utilizing 1-(bromomethyl)-4-fluorobenzene A-8 and reaction conditions of 0° C. to RT for 2 h. Compound A-9 was obtained in 52% yield as a gummy solid liquid following silica gel column chromatography (elution with 0-7% EtOAc in hexane). TLC system: EtOAc/hexane (10:90), R_{f} value=0.5; ¹H NMR (400 MHz, CDCl₃) δ ppm: 10.26 (s, 1H), 7.64 (d, J=7.2 Hz, 1H), 7.53 (d, J=8.0 Hz, 1H), 7.36-7.29 (m, 3H), 7.09-7.05 (m, 2H), 6.99 (t, J=8.4 Hz, 2H), 5.37 (s, 2H); LCMS (ESI) m/z 254.1 [C₁₆H₁₂FNO+H]⁺.

### Synthesis of tert-butyl (1-(1-(4-fluorobenzyl)-1H-indol-4-yl)-5,8,11-trioxa-2-azatridecan-13-yl)carbamate as formic acid salt (compound 84)

Compound 84 was obtained following the reductive alkylation procedure similar to that for the synthesis of compound 1, utilizing tert-butyl (2-(2-(2-(2-aminoethoxy)ethoxy)ethoxy)ethyl)carbamate and reaction conditions of imine formation at 70° C. for 24 h and NaBH₄ reduction at 0° C. to RT for 1 h. Compound 84 was obtained in 16% yield as a colourless gum following reversed phase column chromatography (elution with 15% ACN in 0.1% FA in H₂O). TLC system: MeOH/CH₂Cl₂ (10:90), R_{f} value=0.2; ¹H NMR (400 MHz, DMSO-d₆-D₂O) δ ppm: 8.36 (s, 1H; HCOOH), 7.59 (d, J=3.2 Hz, 1H), 7.50 (dd, J=7.2 Hz, 1.2 Hz, 1H), 7.30-7.27 (m, 2H), 7.20-7.11 (m, 4H), 6.72 (d, J=3.2 Hz, 1H), 5.42 (s, 2H), 4.34 (s, 2H), 3.67 (t, J=4.8 Hz, 2H), 3.56 (s, 4H), 3.51-3.47 (m, 4H), 3.35 (t, J=6.0 Hz, 2H), 3.10-3.04 (m, 4H), 1.36 (s, 9H); LCMS (ESI) m/z 530.4 [C₂₉H₄₀FN₃O₅+H]⁺.

### Example 20: Synthesis of Compound 85 Via Synthesis Route A

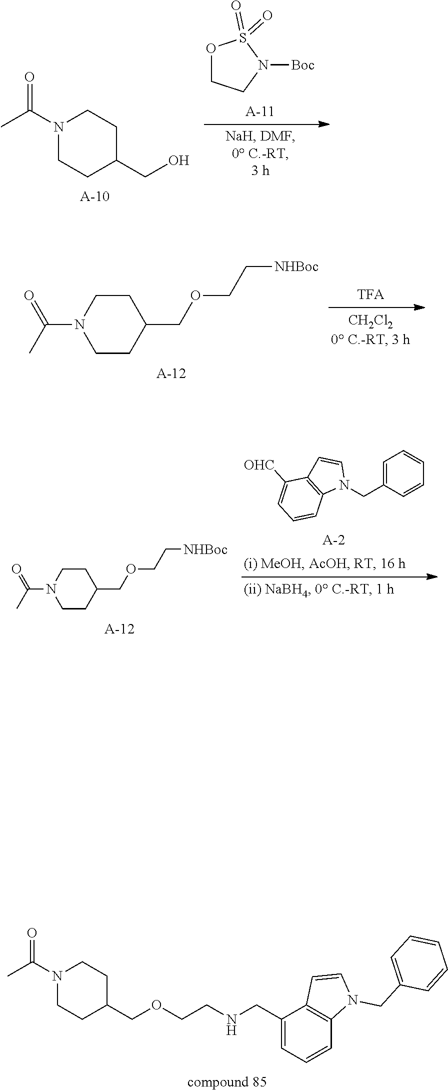 [SMILES: CC(=O)N1CCC(CO)CC1 ; CC(C)(C)OC(=O)N1CCOS1(=O)=O ; CC(=O)N1CCC(COCCNC(=O)OC(C)(C)C)CC1 ; O=Cc1cccc2c1ccn2Cc1ccccc1 ; CC(=O)N1CCC(COCCNC(=O)OC(C)(C)C)CC1 ; CC(=O)N1CCC(COCCNCc2cccc3c2ccn3Cc2ccccc2)CC1]

### Synthesis of tert-butyl (2-((1-acetylpiperidin-4-yl)methoxy)ethyl)carbamate (A-12)

To a stirred solution of 1-(4-(hydroxymethyl)piperidin-1-yl)ethan-1-one A-10 (1.0 g, 6.36 mmol, 1 equiv.) in DMF (20 mL) at 0° C., was added NaH (60% dispersion in mineral oil) (382 mg, 9.54 mmol, 1.5 equiv.). The reaction mixture was stirred at 0° C. for 30 min before tert-butyl 1,2,3-oxathiazolidine-3-carboxylate 2,2-dioxide A-11 (1.56 g, 7.00 mmol, 1.1 equiv.) was added. The resultant mixture was warmed to RT and stirred at RT for 3 h. Upon the completion of reaction by TLC, the reaction mixture was quenched with ice-cold water (80 mL) and extracted with EtOAc (3×50 mL). The combined organic layers were dried over Na₂SO₄, concentrated under reduced pressure, and purified by silica gel (100-200 mesh) column chromatography (elution with 2% MeOH in CH₂Cl₂) to afford A-12 as a colourless gummy solid (0.9 g, yield: 47%). TLC system: EtOAc/hexane (70:30), R_{f} value=0.3; LCMS (ESI) m/z 301.7 [C₁₅H₂₈N₂O₄+H]⁺.

### Synthesis of 1-(4-((2-aminoethoxy)methyl)piperidin-1-yl)ethan-1-one (A-13)

To a stirred solution of A-12 (0.9 g, 3.00 mmol, 1.0 equiv.) in CH₂Cl₂ (9 mL) at 0° C., was added TFA (1.8 mL). The resultant mixture was warmed to RT and stirred at RT for 3 h. Upon the completion of reaction by TLC, the volatiles were removed under vacuum. The residue was diluted with water, basified with sat. aq. NaHCO₃ solution and extracted with 20% MeOH in CH₂Cl₂ (3×30 mL). The combined organic layers were dried over Na₂SO₄ and concentrated under reduced pressure to afford A-13 as a pale brown gummy liquid (250 mg, crude). TLC system: MeOH/CH₂Cl₂ (10:90), R_{f} value=0.2; LCMS (ESI) m/z 201.2 [C₁₀H₂₀N₂O₂+H]⁺. The crude material was used directly in the next step without further purification.

### Synthesis of 1-(4-((2-(((1-benzyl-1H-indol-4-yl)methyl)amino)ethoxy)methyl)piperidin-1-yl)ethan-1-one as formic acid salt (Compound 85)

Compound 85 was obtained following the reductive alkylation procedure similar to that for the synthesis of compound 1, utilizing carbaldehyde A-2 and reaction conditions of imine formation at RT for 16 h and NaBH₄ reduction at 0° C. to RT for 1 h. Compound 85 was obtained in 15% yield as a colourless gum following reversed phase column chromatography (elution with 20% ACN in 0.1% FA in H₂O). TLC system: MeOH/CH₂Cl₂ (10:90), R_{f} value=0.3; ¹H NMR (400 MHz, DMSO-d₆) δ ppm: 8.21 (s, 1H; HCOOH), 7.49 (d, J=3.2 Hz, 1H), 7.35-7.28 (m, 3H), 7.25-7.23 (m, 1H), 7.22-7.18 (m, 2H), 7.07-7.00 (m, 2H), 6.59 (d, J=2.8 Hz, 1H), 5.41 (s, 2H), 4.35-4.32 (m, 1H), 4.01 (s, 2H), 3.78-3.74 (m, 1H), 3.47 (t, J=5.6 Hz, 2H), 3.21 (d, J=6.4 Hz, 2H), 2.99-2.93 (m, 1H), 2.75 (t, J=5.6 Hz, 2H), 2.46-2.43 (m, 1H), 1.96 (s, 3H), 1.78-1.71 (m, 1H), 1.67-1.59 (m, 2H), 1.10-0.89 (m, 2H); LCMS (ESI) m/z 420.3 [C₂₆H₃₃N₃O₂+H]⁺.

### Example 21: Synthesis of Compound 87 Via Synthesis Route B

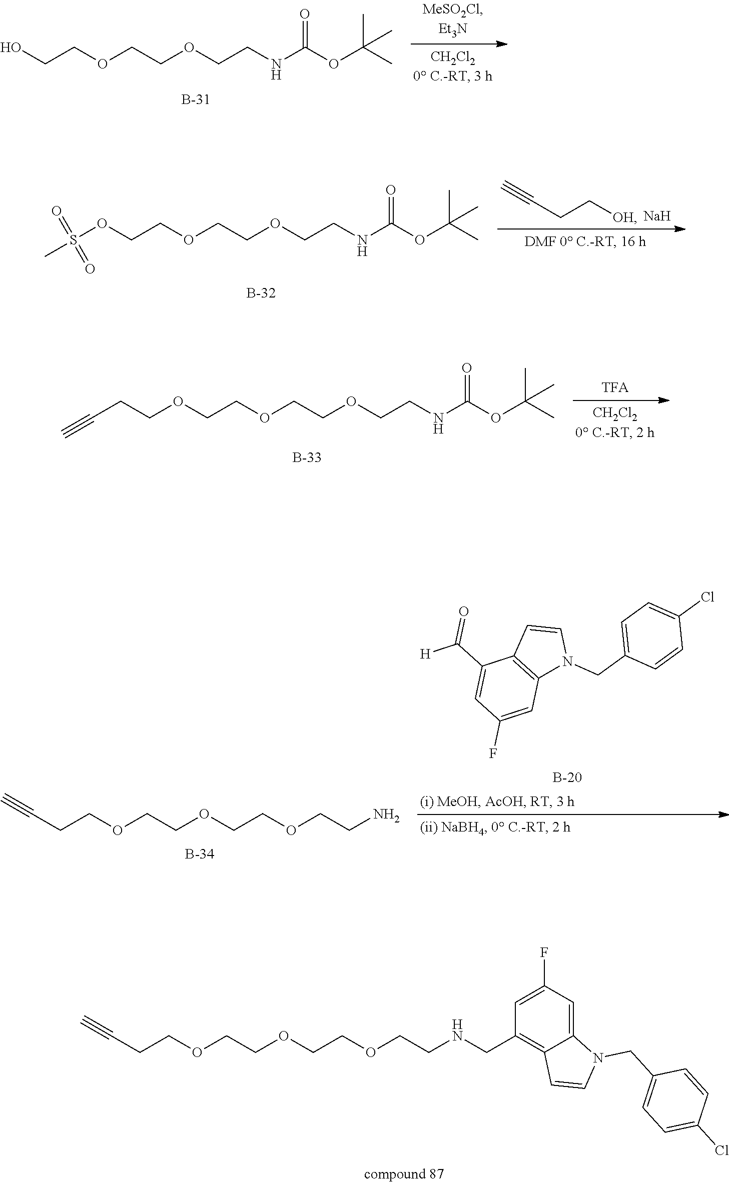

### Synthesis of 2,2-dimethyl-4-oxo-3,8,11-trioxa-5-azatridecan-13-yl methanesulfonate (B-32)

To a stirred solution of tert-butyl (2-(2-(2-hydroxyethoxy)ethoxy)ethyl)carbamate B-31 (5.0 g, 20.1 mmol, 1 equiv.) in CH₂Cl₂ (50 mL) at 0° C., was added Et₃N (8.4 mL, 60.3 mmol, 3 equiv.) followed by addition of MeSO₂Cl (2.3 mL, 30.2 mmol, 1.5 equiv.) dropwise. The resultant mixture was warmed to RT and stirred at RT for 3 h. Upon the completion of reaction by TLC, the reaction mixture was diluted with water (50 mL) and extracted with CH₂Cl₂ (2×50 mL). The combined organic layers were washed with brine, dried over anhydrous Na₂SO₄, filtered, and concentrated under reduced pressure to afford B-32 (5.00 g, yield: 76%) as a pale-yellow liquid in its crude. TLC system: 100% EtOAc, R_{f} value=0.7; ¹H NMR (400 MHz, CDCl₃) δ ppm: 4.97 (br. s, 1H), 4.41-4.38 (m, 2H), 3.79-3.76 (m, 2H), 3.68-3.61 (m, 4H), 3.55-3.52 (m, 2H), 3.34-3.25 (m, 2H), 3.08 (s, 3H), 1.45 (s, 9H). The crude material was used directly in the next step without further purification.

### Synthesis of tert-butyl (2-(2-(2-(but-3-yn-1-yloxy)ethoxy)ethoxy)ethyl)carbamate (B-33)

To a stirred solution of B-32 (1.0 g, 3.05 mmol, 1 equiv.) in DMF (10 mL) at 0° C., was added NaH (244 mg, 60% in mineral oil, 6.10 mmol, 2 equiv.). The mixture was stirred at 0° C. for 30 min before but-3-yn-1-ol (277 μL, 3.66 mmol, 1.2 equiv.) in DMF (1 mL) was added dropwise at 0° C. The resultant mixture was allowed to warm to RT and stirred at RT for 16 h. Upon the completion of reaction by TLC, the reaction mixture was quenched with saturated aq. NH₄Cl (50 mL) and extracted with CH₂Cl₂ (2×30 mL). The combined organic layers were washed with water, then brine, dried over anhydrous Na₂SO₄, filtered, and concentrated under reduced pressure. The crude material obtained was purified by silica gel (100-200 mesh) column chromatography (gradient elution with 0-70% EtOAc in hexanes) to afford B-33 (600 mg, yield: 65%) as a pale-yellow liquid. TLC system: EtOAc/hexane (70:30), R_{f} value=0.8; ¹H NMR (400 MHz, CDCl₃) δ ppm: 5.09 (br. s, 1H), 3.66-3.61 (m, 10H), 3.56-3.53 (m, 2H), 3.32-3.30 (m, 2H), 2.51-2.45 (m, 2H), 1.98 (t, J=2.8 Hz, 1H), 1.46 (s, 9H).

### Synthesis of 2-(2-(2-(but-3-yn-1-yloxy)ethoxy)ethoxy)ethan-1-amine (B-34)

To a stirred solution of B-33 (900 mg, 2.99 mmol) in CH₂Cl₂ (9 mL) at 0° C., was added TFA (5 mL). The resultant mixture was warmed to RT and stirred at RT for 2 h. Upon the completion of reaction by TLC, the reaction mixture was concentrated in vacuo. The obtained residue was basified with aqueous NaHCO₃ solution and extracted with CH₂Cl₂ (2×30 mL). The combined organic layers were washed with water, then brine, dried over Na₂SO₄, filtered, and concentrated under reduced pressure to afford B-34 (500 mg, yield: 83%) as a pale-yellow liquid in its crude. TLC system: 100% EtOAc, R_{f} value=0.3; ¹H NMR (400 MHz, DMSO-d₆) δ ppm: 3.56-3.46 (m, 10H), 3.35 (t, J=5.6 Hz, 2H), 2.79 (t, J=2.8 Hz, 1H), 2.68-2.64 (m, 2H), 2.41-2.36 (m, 2H). The crude material was used directly in the next step without further purification.

### Synthesis of 2-(2-(2-(but-3-yn-1-yloxy)ethoxy)ethoxy)-N-((1-(4-chlorobenzyl)-6-fluoro-1H-indol-4-yl)methyl)ethan-1-amine (Compound 87)

Compound 87 was obtained following the reductive alkylation procedure similar to that for the synthesis of compound 1, utilizing carbaldehyde B-20 (synthesized via example 8) and reaction conditions of imine formation at RT for 3 h and NaBH₄ reduction at 0° C. to RT for 2 h. Compound 87 was obtained in 13% yield as a colourless liquid following prep-HPLC [Prep conditions: Gemini C18 column, 5 μm, 21.2×250 mm; Buffer A: 0.1% liquid NH₃ in H₂O; Buffer B: 80:20 MeCN/H₂O; Gradient (Time/% B): 0/70, 10/90, 16/90, 16.1/98; Flow Rate: 14 mL/min] purification. TLC system: EtOAc/hexane (8:2), R_{f} value=0.3; ¹H NMR (400 MHz, DMSO-d₆) δ ppm: 7.47 (d, J=3.2 Hz, 1H), 7.39-7.36 (m, 2H), 7.23-7.18 (m, 3H), 6.89 (dd, J=10.8 Hz, 2.0 Hz, 1H), 6.59 (d, J=2.8 Hz, 1H), 5.38 (s, 2H), 3.95 (s, 2H), 3.52-3.44 (m, 12H), 2.78 (t, J=2.8 Hz, 1H), 2.69 (t, J=5.6 Hz, 2H), 2.38-2.34 (m, 2H); LCMS (ESI) m/z 473.2 [C₂₆H₃₀ClFN₂O₃+H]⁺.

### Example 22: Synthesis of Compound 101 Via Synthesis Route K

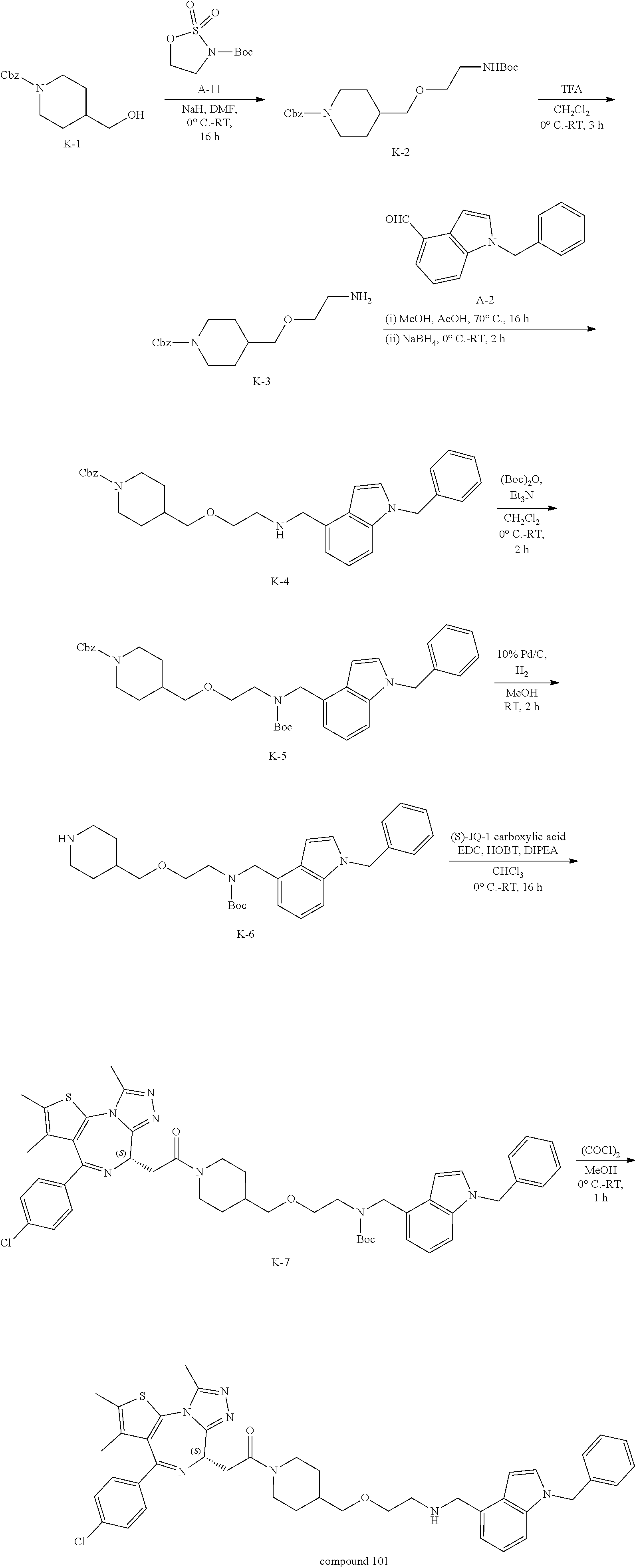

### Synthesis of benzyl 4-((2-((tert-butoxycarbonyl)amino)ethoxy)methyl)piperidine-1-carboxylate (K-2)

To a stirred solution of benzyl 4-(hydroxymethyl)piperidine-1-carboxylate K-1 (1.20 g, 4.81 mmol, 1 equiv.) in DMF (12 mL) at 0° C., was added NaH (60% dispersion in mineral oil) (385 mg, 9.62 mmol, 2.0 equiv.). The reaction mixture was stirred at 0° C. for 20 min before tert-butyl 1,2,3-oxathiazolidine-3-carboxylate 2,2-dioxide A-11 (1.61 g, 7.22 mmol, 1.5 equiv.) was added. The resultant mixture was warmed to RT and stirred at RT for 16 h. Upon the completion of reaction by TLC, the reaction mixture was quenched with ice-cold water (50 mL) and extracted with EtOAc (2×70 mL). The combined organic layers were dried over Na₂SO₄, concentrated under reduced pressure, and purified by silica gel (100-200 mesh) column chromatography (elution with 30-35% EtOAc in hexane) to afford K-2 as a brown gummy solid (1.40 g, yield: 74%). TLC system: EtOAc/hexane (40:60), R_{f} value=0.2; ¹H NMR (400 MHz, CDCl₃) δ ppm: 7.38-7.28 (m, 5H), 5.12 (s, 2H), 4.81 (br. s, 1H), 4.20 (br. s, 2H), 3.46 (t, J=5.2 Hz, 2H), 3.30-3.27 (m, 4H), 2.78 (t→br, 2H), 1.79-1.70 (m, 3H), 1.44 (s, 9H), 1.27-1.21 (m, 2H); LCMS (ESI) m/z 393.4 [C₂₁H₃₂N₂O₅+H]⁺.

### Synthesis of benzyl 4-((2-aminoethoxy)methyl)piperidine-1-carboxylate (K-3)

To a stirred solution of K-2 (1.40 g, 3.57 mmol, 1.0 equiv.) in CH₂Cl₂ (14 mL) at 0° C., was added TFA (2.8 mL). The resultant mixture was warmed to RT and stirred at RT for 3 h. Upon the completion of reaction by TLC, the volatiles were removed under vacuum. The residue was diluted with water, basified with sat. aq. NaHCO₃ solution and extracted with 10% MeOH in CH₂Cl₂ (2×100 mL). The combined organic layers were dried over Na₂SO₄ and concentrated under reduced pressure to afford K-3 as a colourless gummy liquid (850 mg, 80%). TLC system: MeOH/CH₂Cl₂ (10:90), R_{f} value=0.2; ¹H NMR (400 MHz, CDCl₃) δ ppm: 7.36-7.29 (m, 5H), 5.11 (s, 2H), 4.18 (br. s, 2H), 3.55 (t, J=5.2 Hz, 2H), 3.51-3.48 (br. s, 2H), 3.30 (d, J=6.0 Hz, 2H), 3.00 (t, J=5.2 Hz, 2H), 2.77 (t→br, 2H), 1.78-1.70 (m, 3H), 1.18-1.15 (m, 2H); LCMS (ESI) m/z 293.3 [C₁₆H₂₄N₂O₃+H]⁺. The crude material was used directly in the next step without further purification.

### Synthesis of benzyl 4-((2-(((1-benzyl-1H-indol-4-yl)methyl)amino)ethoxy)methyl)piperidine-1-carboxylate (K-4)

To a stirred solution of carbaldehyde A-2 (600 mg, 2.55 mmol, 1.0 equiv.) in MeOH (12 mL) at RT, was added K-3 (895 mg, 3.06 mmol, 1.2 equiv.) and AcOH (0.44 mL, 7.65 mmol, 3.0 equiv.). The resultant mixture was stirred at 70° C. for 16 h before the reaction mixture was cooled to 0° C. and NaBH₄ (193 mg, 5.10 mmol, 2.0 equiv.) was added. The resultant mixture was warmed to RT and stirred at RT for 2 h. Upon the completion of reaction by TLC, the reaction mixture was quenched with ice-cold water (30 mL) and extracted with 10% MeOH in CH₂Cl₂ (2×70 mL). The combined organic layers were dried over Na₂SO₄, concentrated under reduced pressure, and purified by reverse phase column chromatography (Grace) (elution with 30% ACN in 0.1% FA in H₂O) to afford K-4 as a colourless gummy liquid (380 mg, yield: 29%). TLC system: MeOH/CH₂Cl₂ (10:90), R_{f} value=0.25; ¹H NMR (400 MHz, CDCl₃) δ ppm: 7.38-7.35 (m, 5H), 7.33-7.27 (m, 5H), 7.21 (d, J=3.2 Hz, 1H), 7.19-7.14 (m, 2H), 7.10-7.08 (m, 2H), 6.66 (d, J=3.2 Hz, 1H), 5.32 (s, 2H), 5.12 (s, 2H), 4.41 (s, 2H), 4.14 (br. s, 2H), 3.62 (t→br, 2H), 3.22-3.09 (m, 4H→19H; due to moisture peak shift), 2.71-2.66 (m, 2H), 1.72-1.67 (m, 1H), 1.61-1.57 (m, 2H), 1.11-1.02 (m, 2H); LCMS (ESI) m/z 512.3 [C₃₂H₃₇N₃O₃+H]⁺.

### Synthesis of benzyl 4-((2-(((1-benzyl-1H-indol-4-yl)methyl)(tert-butoxycarbonyl)amino)ethoxy)methyl)piperidine-1-carboxylate (K-5)

To a stirred solution of K-4 (380 mg, 0.74 mmol, 1.0 equiv.) in CH₂Cl₂ (8 mL) at 0° C., was added Et₃N (0.21 mL, 1.48 mmol, 2.0 equiv.) and (Boc)₂O (0.26 mL, 1.11 mmol, 1.5 equiv.). The resultant mixture was warmed to RT and stirred at RT for 2 h. Upon the completion of reaction by TLC, the reaction mixture was evaporated and purified by silica gel (60-120 mesh) column chromatography (elution with 70-80% EtOAc in hexane) to afford K-5 as a colourless liquid (390 mg, yield: 86%). TLC system: MeOH/CH₂Cl₂ (10:90), R_{f} value=0.5; ¹H NMR (400 MHz, CDCl₃) δ ppm: 7.36-7.34 (m, 4H), 7.33-7.27 (m, 4H), 7.20 (d, J=6.0 Hz, 1H), 7.14-7.09 (m, 4H), 6.97-6.93 (m, 1H), 6.64-6.56 (m, 1H), 5.31 (s, 2H), 5.12 (s, 2H), 4.83 (s, 2H), 4.17 (br. s, 2H), 3.51-3.47 (m, 1H), 3.40 (br. s, 2H), 3.28-3.27 (m, 1H), 3.21-3.18 (m, 2H), 2.77-2.73 (m, 2H), 1.60-1.67 (m, 3H), 1.50-1.46 (m, 9H), 1.15-1.12 (m, 2H); LCMS (ESI) m/z 612.3 [C₃₇H₄₅N₃O₅+H]⁺.

### Synthesis of tert-butyl ((1-benzyl-1H-indol-4-yl)methyl)(2-(piperidin-4-ylmethoxy)ethyl)carbamate (K-6)

To a stirred solution of K-5 (380 mg, 0.62 mmol, 1.0 equiv.) in MeOH (8 mL) at RT was added 10% Pd/C (80 mg). The mixture was stirred at RT for 2 h under a H₂-balloon. Upon the completion of reaction by TLC, the reaction mixture was filtered through a Celite pad, washed with 5% MeOH in CH₂Cl₂ (30 mL), and evaporated under vacuum to afford K-6 as a light-yellow gum (250 mg, crude). TLC system: MeOH/CH₂Cl₂ (5:95), R_{f} value=0.2; ¹H NMR (400 MHz, CDCl₃) δ ppm: 7.32-7.27 (m, 3H), 7.22-7.20 (m, 1H), 7.13-7.10 (m, 4H), 6.97-6.95 (m, 1H), 6.65-6.58 (m, 1H), 5.32 (s, 2H), 4.84 (s, 2H), 3.53-3.51 (br. s, 1H), 3.42 (br. s, 2H), 3.28-3.18 (m, 3H), 3.08-3.05 (m, 2H), 2.63-2.55 (m, 2H), 1.70-1.67 (m, 3H), 1.51-1.45 (m, 9H), 1.13-1.10 (m, 2H); LCMS (ESI) m/z 478.2 [C₂₉H₃₉N₃O₃+H]⁺. The crude material was used directly in the next step without further purification.

### Synthesis of tert-butyl (S)-((1-benzyl-1H-indol-4-yl)methyl)(2-((1-(2-(4-(4-chlorophenyl)-2,3,9-trimethyl-6H-thieno[3,2-f][1,2,4]triazolo[4,3-a][1,4]diazepin-6-yl)acetyl)piperidin-4-yl)methoxy)ethyl)carbamate (K-7)

To a stirred solution of (S)-JQ-1 carboxylic acid (commercially procured) (170 mg, 0.42 mmol, 1.0 equiv.) in CHCl₃ (3.5 mL) at 0° C., was added K-6 (239 mg, 0.50 mmol, 1.2 equiv.), DIPEA (0.12 mL, 1.26 mmol, 3.0 equiv.), HOBt (74 mg, 0.55 mmol, 1.3 equiv.) and EDC·HCl (96 mg, 0.50 mmol, 1.2 equiv.). The resultant mixture was warmed to RT and stirred at RT for 16 h. Upon the completion of reaction by TLC, the reaction mixture was quenched with ice-cold water (30 mL) and extracted with CH₂Cl₂ (2×50 mL). The combined organic layers were dried over Na₂SO₄, filtered, and concentrated under reduced pressure to afford crude product. The crude product was purified by reverse phase column chromatography (Grace) (elution with 60-65% ACN in 0.1% FA in H₂O) to afford K-7 as a light-yellow solid (300 mg, yield: 83%). TLC system: MeOH/CH₂Cl₂ (10:90), R_{f} value=0.7; ¹H NMR (400 MHz, CDCl₃) δ ppm: 7.42-7.39 (m, 2H), 7.33-7.27 (m, 5H), 7.21 (d, J=8.0 Hz, 1H), 7.14-7.10 (m, 4H), 6.99-6.94 (br. s, 1H), 6.65-6.58 (m, 1H), 5.32 (s, 2H), 4.84-4.79 (m, 3H), 4.62 (t→br, 1H), 4.27-4.22 (m, 1H), 3.69-3.52 (m, 3H), 3.42 (br. s, 2H), 3.30-3.10 (m, 4H), 2.66 (s, 3H), 2.64-2.60 (m, 1H), 2.39 (s, 3H), 1.84-1.73 (m, 3H), 1.67 (s, 3H), 1.52-1.45 (m, 9H), 1.29-1.26 (m, 2H); LCMS (ESI) m/z 860.3 [C₄₈H₅₄ClN₇O₄S+H]⁺.

### Synthesis of (S)-1-(4-((2-(((1-benzyl-1H-indol-4-yl)methyl)amino)ethoxy)methyl)piperidin-1-yl)-2-(4-(4-chlorophenyl)-2,3,9-trimethyl-6H-thieno[3,2-f][1,2,4]triazolo[4,3-a][1,4]diazepin-6-yl)ethan-1-one (compound 101)

To a stirred solution of K-7 (150 mg, 0.17 mmol, 1.0 equiv.) in MeOH (1.5 mL) at 0° C., was added oxalyl chloride (30 μL, 0.35 mmol, 2.0 equiv.). The resultant mixture was warmed to RT and stirred at RT for 1 h. Upon the completion of reaction by TLC, the volatiles were removed under reduced pressure and the crude was purified by reverse phase column chromatography (elution with 40-45% ACN in 0.1% FA in H₂O) to afford 40 mg of isolated product as formic acid salt. The isolated product was quenched with sat. NaHCO₃ solution, extracted with EtOAc, concentrated, triturated with Et₂O and pentane, and lyophilized to afford compound 101 as an off-white solid (21 mg, 16%). TLC system: MeOH/CH₂Cl₂ (10:90), R_{f} value=0.6; melting point range: 82-84° C.; ¹H NMR (400 MHz, DMSO-d₆) δ ppm: 7.50-7.47 (m, 3H), 7.44-7.42 (m, 2H), 7.32-7.27 (m, 3H), 7.24-7.22 (m, 1H), 7.20-7.17 (m, 2H), 7.05-7.01 (m, 1H), 6.99-6.98 (m, 1H), 6.58 (d, J=3.2 Hz, 1H), 5.40 (s, 2H), 4.57 (t, J=7.2 Hz, 1H), 4.36-4.33 (m, 1H), 4.13-4.09 (m, 1H), 3.97 (s, 2H), 3.60-3.54 (m, 1H), 3.47-3.46 (m, 2H), 3.40-3.37 (m, 2H), 3.25-3.22 (m, 2H), 3.12-3.06 (m, 1H), 2.71-2.70 (m, 2H), 2.59 (s, 3H), 2.41 (s, 3H), 1.79-1.73 (m, 2H), 1.63-1.62 (m, 4H), 1.24-1.16 (m, 1H), 0.97-0.94 (m, 1H); LCMS (ESI) m/z 760.3 [C₄₃H₄₆ClN₇O₂S+H]⁺.

### Example 23: Synthesis of Compound 107 Via Synthesis Route K

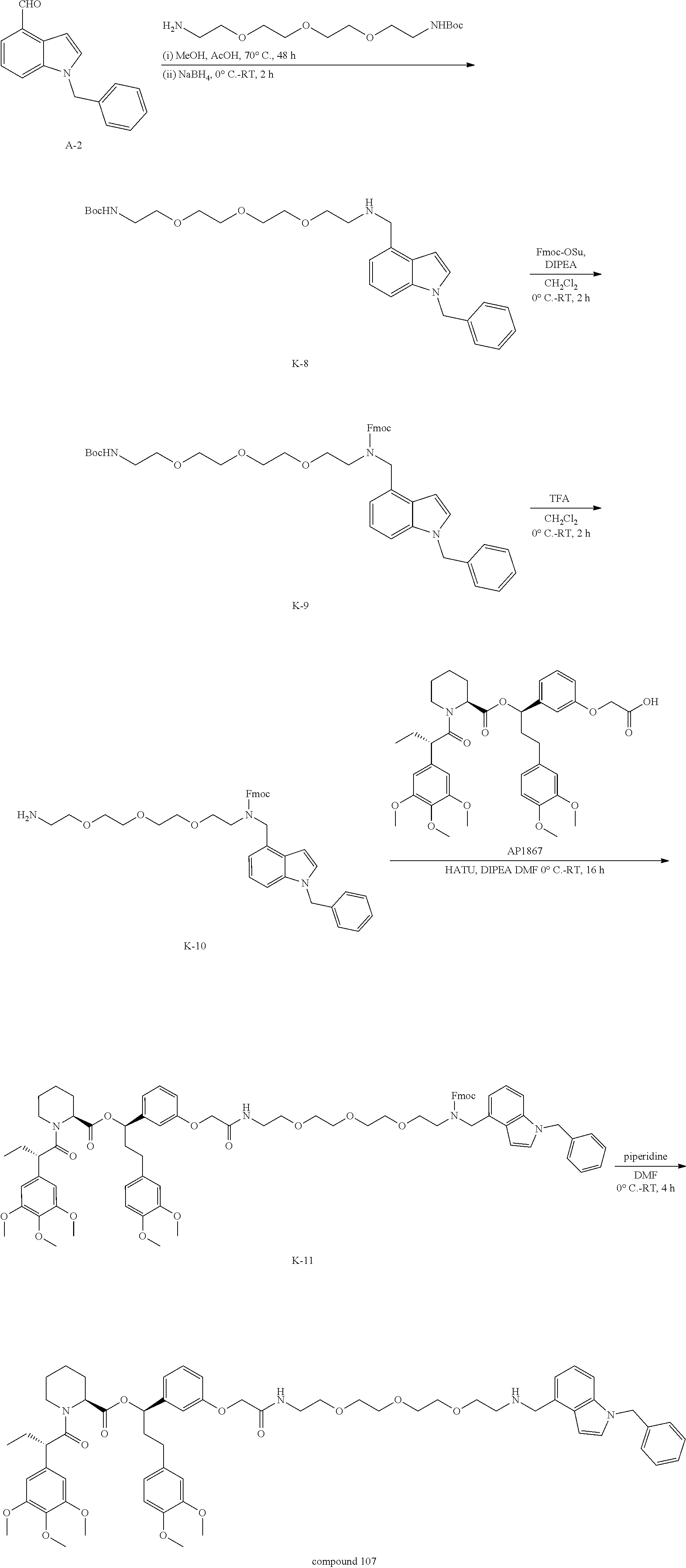

### Synthesis of tert-butyl (1-(1-benzyl-1H-indol-4-yl)-5,8,11-trioxa-2-azatridecan-13-yl)carbamate as formic acid salt (K-8)

To a stirred solution of A-2 (1.00 g, 4.25 mmol, 1 equiv.) in MeOH (20 mL) at RT, was added tert-butyl (2-(2-(2-(2-aminoethoxy)ethoxy)ethoxy)ethyl)carbamate (1.24 g, 4.25 mmol, 1 equiv.), AcOH (73 μL, 1.28 mmol, 0.3 equiv.) and molecular sieves (200 mg). The resultant mixture was stirred at 70° C. for 48 h before it was cooled to 0° C. and NaBH₄ (322 mg, 8.50 mmol, 2 equiv.) was added portion-wise. The reaction mixture was warmed to RT and stirred at RT for 2 h. Upon the completion of reaction by TLC, the reaction mixture was quenched with ice-cold water (30 mL) and extracted with 10% MeOH in CH₂Cl₂ (2×50 mL). The combined organic layers were dried over Na₂SO₄, concentrated under reduced pressure, and purified by reverse phase column chromatography (Grace) (elution with 30-35% ACN in 0.1% FA in H₂O) to afford K-8 (formic acid salt) as a brown gummy liquid (0.90 g, yield: 38%). TLC system: MeOH/CH₂Cl₂ (5:95), R_{f} value=0.2; ¹H NMR (400 MHz, CDCl₃) δ ppm: 8.46 (s, 1H; HCOOH), 7.30-7.27 (m, 5H), 7.21-7.15 (m, 3H), 7.11 (d, J=7.2 Hz, 2H), 6.70 (d, J=2.8 Hz, 1H), 5.47 (s, 1H), 5.33 (s, 2H), 4.42 (s, 2H), 3.73 (t, J=4.8 Hz, 2H), 3.64-3.45 (m, 12H), 3.09 (t, J=4.8 Hz, 2H), 1.42 (s, 9H); LCMS (ESI) m/z 512.3 [C₂₉H₄₁N₃O₅+H]⁺.

### Synthesis of (9H-fluoren-9-yl)methyl ((1-benzyl-1H-indol-4-yl)methyl)(2,2-dimethyl-4-oxo-3,8,11,14-tetraoxa-5-azahexadecan-16-yl)carbamate (K-9)

To a stirred solution of K-8 (900 mg, 1.61 mmol, 1 equiv.) in CH₂Cl₂ (9 mL) at 0° C., was added DIPEA (0.56 mL, 3.22 mmol, 2 equiv.) and Fmoc-OSu, (651 mg, 1.93 mmol, 1.2 equiv.). The resultant mixture was warmed to RT and stirred at RT for 2 h. Upon the completion of reaction by TLC, the reaction mixture was quenched with ice-cold water (10 mL) and extracted with CH₂Cl₂ (2×30 mL). The combined organic layers were dried over Na₂SO₄, filtered, and concentrated under reduced pressure to afford crude product. The crude product was purified by silica gel (60-120 mesh) column chromatography (elution with 30-35% EtOAc in hexane) to afford K-9 as a light-green gummy liquid (1.00 g, yield: 85%). TLC system: EtOAc/hexane (30:70), R_{f} value=0.15; ¹H NMR (400 MHz, CDCl₃) δ ppm: 7.75 (d, J=7.2 Hz, 1H), 7.69 (d, J=7.6 Hz, 1H), 7.60 (d, J=7.2 Hz, 1H), 7.40-7.37 (m, 2H), 7.31-7.28 (m, 5H), 7.21 (t, J=8.0 Hz, 1H), 7.13-7.10 (m, 5H), 6.93-6.88 (m, 1H), 6.61-6.51 (d, 1H), 5.33 (d, J=12.4 Hz, 2H), 5.00 (br. s, 1H), 4.89 (d, J=18.8 Hz, 2H), 4.59 (d, J=6.0 Hz, 1H), 4.45 (d, J=6.0 Hz, 1H), 4.28-4.15 (m, 1H), 3.64-3.56 (m, 9H), 3.51-3.42 (m, 3H), 3.29-3.24 (m, 4H), 1.42 (s, 9H); LCMS (ESI) m z 734.4 [C₄₄H₅₁N₃O₇+H]⁺.

### Synthesis of (9H-fluoren-9-yl)methyl (2-(2-(2-(2-aminoethoxy)ethoxy)ethoxy)ethyl)((1-benzyl-1H-indol-4-yl)methyl)carbamate as trifluoroacetic acid salt (K-10)

To a stirred solution of K-9 (400 mg, 0.55 mmol, 1 equiv.) in CH₂Cl₂ (5 mL) at 0° C., was added TFA (1 mL). The resultant mixture was warmed to RT and stirred at RT for 2 h. Upon the completion of reaction by TLC, the volatiles were removed under reduced pressure and the material obtained was triturated with n-pentane (2×10 mL) to afford K-10 (trifluoroacetic acid salt) as a light-brown liquid (450 mg, crude). TLC system: MeOH/CH₂Cl₂ (10:90), R_{f} value=0.2; LCMS (ESI) m/z 634.3 [C₃₉H₄₃N₃O₅+H]⁺. The crude material was used directly in the next step without further purification.

### Synthesis of (R)-1-(3-((4-((1-benzyl-1H-indol-4-yl)methyl)-1-(9H-fluoren-9-yl)-3,17-dioxo-2,7,10,13-tetraoxa-4,16-diazaoctadecan-18-yl)oxy)phenyl)-3-(3,4-dimethoxyphenyl)propyl (S)-1-((S)-2-(3,4,5-trimethoxyphenyl)butanoyl)piperidine-2-carboxylate (K-11)

To a stirred solution of AP1867 (commercially procured) (100 mg, 0.14 mmol, 1.0 equiv.) in DMF (2.5 mL) at 0° C., was added K-10 (157 mg, 0.21 mmol, 1.5 equiv.), HATU (65 mg, 0.17 mmol, 1.2 equiv.) and DIPEA (61 μL, 0.35 mmol, 2.5 equiv.). The resultant mixture was warmed to RT and stirred at RT for 16 h. Upon the completion of reaction by TLC, the reaction mixture was quenched with ice-cold water (10 mL) and extracted with EtOAc (3×30 mL). The combined organic layers were dried over Na₂SO₄, filtered, and concentrated under reduced pressure to afford the crude compound. The crude compound was purified by silica gel (100-200 mesh) column chromatography (elution with EtOAc and 2% MeOH in CH₂Cl₂) to afford K-11 as a brown gummy liquid (136 mg, yield: 74%). TLC system: EtOAc, R_{f} value=0.6; LCMS (ESI) m/z 1310.0 [C₇₇H₈₈N₄O₁₅+H]⁺.

### Synthesis of (R)-1-(3-((1-(1-benzyl-1H-indol-4-yl)-15-oxo-5,8,11-trioxa-2,14-diazahexadecan-16-yl)oxy)phenyl)-3-(3,4-dimethoxyphenyl)propyl (S)-1-((S)-2-(3,4,5-trimethoxyphenyl)butanoyl)piperidine-2-carboxylate (Compound 107)

To a stirred solution of K-11 (135 mg, 0.1 mmol, 1.0 equiv.) in DMF (2.2 mL) at 0° C., was added piperidine (24 μL, 0.2 mmol, 2.0 equiv.). The resultant mixture was warmed to RT and stirred at RT for 4 h. Upon the completion of reaction by TLC, the reaction mixture was concentrated and purified by prep-HPLC [Prep conditions: Sunfire C18 column, 5 μm, 19×250 mm; Buffer A: 0.1% FA in water; Buffer B: ACN; Gradient (Time/% B): 0/25, 15/60, 25/95; Flow Rate: 13 ml/min] to afford compound 107 as an off-white semi solid (18 mg, yield: 17%). TLC system: MeOH/CH₂Cl₂ (10:90), R_{f} value=0.2; melting point range: 68-72° C.; ¹H NMR (400 MHz, DMSO-d₆) δ ppm: 8.05 (t, J=5.6 Hz, 1H), 7.54 (d, J=2.8 Hz, 1H), 7.45 (br., 1H), 7.31-7.27 (m, 2H), 7.24-7.22 (m, 1H), 7.20-7.15 (m, 3H), 7.12-7.10 (m, 2H), 6.97-6.89 (m, 1H), 6.84-6.81 (m, 3H), 6.75-6.69 (m, 2H), 6.64-6.58 (m, 2H), 6.52 (s, 2H), 5.54-5.51 (m, 1H), 5.43 (s, 2H), 5.29-5.27 (m, 1H), 4.48-4.46 (m, 2H), 4.25 (br. s, 2H), 4.04-3.92 (m, 1H), 3.87-3.82 (m, 1H), 3.74-3.32 (m, 28H), 3.29-3.26 (m, 2H), 2.99 (br. s, 2H), 2.63-2.60 (m, 1H), 2.15-2.13 (m, 1H), 1.94-1.89 (m, 2H), 1.68-1.55 (m, 4H), 1.23-1.17 (m, 1H), 0.80 (t, J=7.6 Hz, 3H); LCMS (ESI) m/z 1087.8 [C₆₂H₇₈N₄O₁₃+H]⁺.

Where absolute stereochemistry has not been indicated, compounds herein are racemic mixtures with relative stereochemistry as drawn.

TABLE 4
Summary of compounds (series I) prepared according to the Examples above and characterizations thereof

| Cpd | Synthesis | | |
|---|---|---|---|
| No. | Route | Structure | NMR and Mass Spectrometry |
| 1 | A | 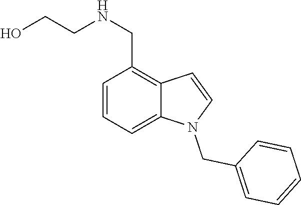 [SMILES: OCCNCc1cccc2c1ccn2Cc1ccccc1] | ¹H NMR (400 MHz, DMSO-d₆) δ (ppm): 8.25 (br. s, 1H; HCOOH), 7.50 (d, J = 3.2 Hz, 1H), 7.36-7.34 (m, 1H), 7.31-7.27 (m, 2H), 7.25- 7.22 (m, 1H), 7.21-7.18 (m, 2H), 7.08-7.03 (m, 2H), 6.62 (dd, J = 3.2 Hz, 0.4 Hz, 1H), 5.42 (s, 2H), 4.05 (s, 2H), 3.52 (t, J = 6.0 Hz, 2H), 2.70 (t, J = 6.0 Hz, 2H). MS (ESI) m/z 281.1[C₁₈H₂₀N₂O + H]⁺. |
| 2 | A | 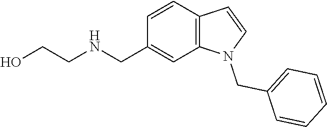 [SMILES: OCCNCc1ccc2ccn(Cc3ccccc3)c2c1] | ¹H NMR (400 MHz, DMSO-d₆) δ (ppm): 8.30 (br. s, 1H; HCOOH), 7.53-7.48 (m, 3H), 7.32-7.28 (m, 2H), 7.26-7.22 (m, 1H), 7.20-7.18 (m, 2H), 7.06 (dd, J = 8.0 Hz, 1.2 Hz, 1H), 6.46 (dd, J = 3.2 Hz, 0.8 Hz, 1H), 5.40 (s, 2H), 3.92 (s, 2H), 3.52 (t, J = 5.6 Hz, 2H), 2.67 (t, |
| | | | J = 5.6 Hz, 2H). MS (ESI) m/z |
| | | | 281.1[C₁₈H₂₀N₂O + H]⁺. |
| 3 | A | 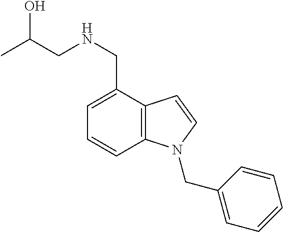 [SMILES: CC(O)CNCc1cccc2c1ccn2Cc1ccccc1] | ¹H NMR (400 MHz, DMSO-d₆) δ (ppm): 8.31 (br. s, 1H; HCOOH), 7.53 (d, J = 3.2 Hz, 1H), 7.40 (dd, J = 6.8 Hz, 2.0 Hz, 1H), 7.32-7.28 (m, 2H), 7.25-7.19 (m, 3H), 7.11- 7.07 (m, 2H), 6.66 (d, J = 2.8 Hz, 1H), 5.42 (s, 2H), 4.14 (s, 2H), 3.85-3.81 (m, 1H), 2.68 (dd, J = 12.0 Hz, 4.0 Hz, 1H), 2.58 (dd, J = 12.0 Hz, 8.0 Hz, 1H), 1.04 (d, J = 6.4 Hz, 3H). MS (ESI) m/z 295.2[C₁₉H₂₂N₂O + H]⁺. |
| 4 | C | 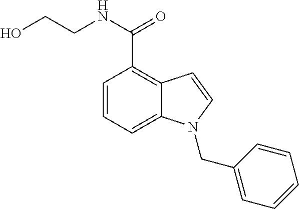 [SMILES: O=C(NCCO)c1cccc2c1ccn2Cc1ccccc1] | ¹H NMR (400 MHz, DMSO-d₆) δ (ppm): 8.12 (t, J = 5.6 Hz, 1H), 7.60-7.58 (m, 2H), 7.41 (d, J = 7.2 Hz, 1H), 7.32-7.28 (m, 2H), 7.25- 7.21 (m, 1H), 7.18-7.12 (m, 3H), 6.89 (d, J = 3.2 Hz, 1H), 5.46 (s, 2H), 4.73 (t, J = 5.6 Hz, 1H), 3.54 (q, J = 6.0 Hz, 2H), 3.37 (q, J = 6.0 Hz, 2H). MS (ESI) m/z 295.2 [C₁₈H₁₈N₂O₂ + H]⁺. |
| 5 | D | 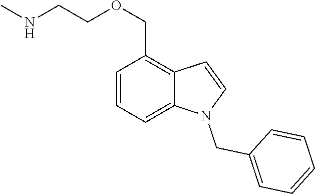 [SMILES: CNCCOCc1cccc2c1ccn2Cc1ccccc1] | ¹H NMR (400 MHz, DMSO-d₆) δ (ppm): 7.53 (d, J = 3.2 Hz, 1H), 7.41 (d, J = 8.0 Hz, 1H), 7.35-7.29 (m, 2H), 7.24 (d, J = 7.2 Hz, 1H), 7.20 (d, J = 7.2 Hz, 2H), 7.08 (t, J = 8.0 Hz, 1H), 7.03 (d, J = 6.8 Hz, 1H), 6.58 (d, J = 2.8 Hz, 1H), 5.43 (s, 2H), 4.76 (s, 2H), 3.61 (t, J = 5.2 Hz, 2H), 2.92 (t, J = 5.2 Hz, 2H), 2.42 (s, 3H). MS (ESI) m/z 295.2[C₁₉H₂₂N₂O + H]⁺. |
| 6 | E | 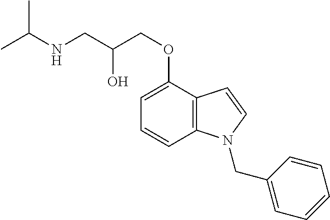 [SMILES: CC(C)NCC(O)COc1cccc2c1ccn2Cc1ccccc1] | ¹H NMR (400 MHz, DMSO-d₆) δ (ppm): 8.31 (s, 1H; HCOOH), 7.38 (d, J = 3.2 Hz, 1H), 7.31-7.27 (m, 2H), 7.25-7.21 (m, 1H), 7.16-7.14 (m, 2H), 7.04-6.97 (m, 2H), 6.52- 6.51 (m, 2H), 5.39 (s, 2H), 4.07- 4.04 (m, 3H), 3.04-2.93 (m, 2H), 2.80 (dd, J = 12.0 Hz, 7.2 Hz, 1H), 1.10 (d, J = 5.6 Hz, 6H). MS (ESI) m/z 339.3[C₂₁H₂₆N₂O₂ + H]⁺. |
| 7 | A | 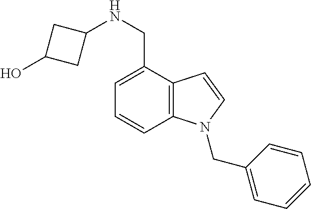 [SMILES: OC1CC(NCc2cccc3c2ccn3Cc2ccccc2)C1] | ¹H NMR (400MHz, DMSO-d₆) δ (ppm): 8.24 (s, 1H; HCOOH), 7.50 (d, J = 3.2 Hz, 1H), 7.34 (d, J = 8.0 Hz, 1H), 7.31-7.27 (m, 2H), 7.25-7.22 (m, 1H), 7.19-7.17 (m, 2H), 7.07-7.00 (m, 2H), 6.60 (d, J = 3.2 Hz, 1H), 5.41 (s, 2H), 3.93- 3.92 (m, 2H), 3.77-3.71 (m, 1H), 2.82-2.76 (m, 1H), 2.46-2.41 (m, 1H), 211-2.07& 1.98-1.93 (m, 1H), 1.69-1.62 (m, 2H). MS (ESI) m/z 307.1[C₂₀H₂₂N₂O + H]⁺. |
| 8 | C | 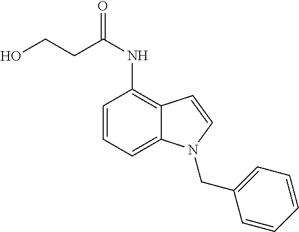 [SMILES: O=C(CCO)Nc1cccc2c1ccn2Cc1ccccc1] | ¹H NMR (400 MHz, DMSO-d₆) δ (ppm):9.57 (s, 1H), 7.66 (d, J = 7.6 Hz, 1H), 7.43 (d, J = 3.2 Hz, 1H), 7.31-7.27 (m, 2H), 7.25-7.21 (m, 1H), 7.17-7.15 (m, 3H), 7.01 (t, J = 8.0 Hz, 1H), 6.75 (d, J = 3.2 Hz, 1H), 5.40 (s, 2H), 4.76 (t, J = 4.8 Hz, 1H), 3.74 (q, J = 6.0 Hz, 2H), 2.59 (t, J = 6.4 Hz, 2H). MS (ESI) m/z 295.1[C₁₈H₁₈N₂O₂ + H]⁺. |
| 9 | B | 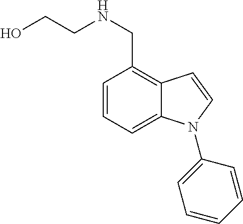 [SMILES: OCCNCc1cccc2c1ccn2-c1ccccc1] | ¹H NMR (400 MHz, DMSO-d₆) δ (ppm): 8.35 (br. s, 1H), 7.69 (d, J = 3.2 Hz, 1H), 7.62-7.57 (m, 4H), 7.50 (d, J = 7.6 Hz, 1H), 7.44-7.39 (m, 1H), 7.24-7.18 (m, 2H), 6.91 (d, J = 2.8 Hz, 1H), 4.21 (s, 2H), 3.59 (t, J = 5.2 Hz, 2H), 2.81 (t, J = 4.8 Hz, 2H). MS (ESI) m/z 267.1[C₁₇H₁₈N₂O + H]⁺. |
| 10 | A | 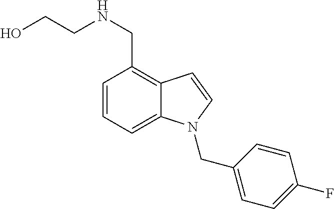 [SMILES: OCCNCc1cccc2c1ccn2Cc1ccc(F)cc1] | ¹H NMR (400 MHz, DMSO-d₆) δ (ppm): 8.26 (br. s, 1H; HCOOH), 7.52 (d, J = 3.2 Hz, 1H), 7.39 (dd, J = 6.8 Hz, 2.0 Hz, 1H), 7.27-7.23 (m, 2H), 7.13 (t, J = 6.8 Hz, 2H), 7.09-7.06 (m, 2H), 6.64 (dd, J = 3.2 Hz, 0.8 Hz, 1H), 5.41 (s, 2H), 4.09 (s, 2H), 3.54 (t, J = 5.6 Hz, 2H), 2.74 (t, J = 5.6 Hz, 2H). MS (ESI) m/z 299.2[C₁₈H₁₉FN₂O + H]⁺. |
| 11 | A | 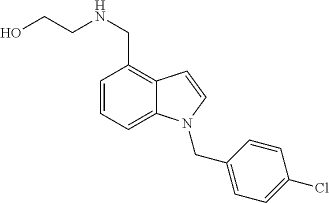 [SMILES: OCCNCc1cccc2c1ccn2Cc1ccc(Cl)cc1] | ¹H NMR (400 MHz, DMSO-d₆- D₂O exchange) δ (ppm): 8.37 (br. s, 1H; HCOOH), 7.59 (d, J = 3.2 Hz, 1H), 7.48-7.45 (m, 1H), 7.37 (d, J = 8.4 Hz, 2H), 7.23 (d, J = 8.4 Hz, 2H), 7.17 (d, J = 4.8 Hz, 2H), 6.73 (d, J = 3.2 Hz, 1H), 5.44 (s, 2H), 4.32 (s, 2H), 3.65 (t, J = 5.2 Hz, 2H), 2.95 (t, J = 5.2 Hz, 2H). MS (ESI) m/z 315.2 [C₁₈H₁9ClN₂O + H]⁺. |
| 12 | A | 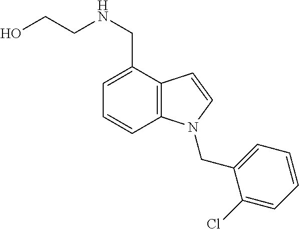 [SMILES: OCCNCc1cccc2c1ccn2Cc1ccccc1Cl] | ¹H NMR (400 MHz, DMSO-d₆) δ (ppm): 8.26 (s, 1H; HCOOH), 7.50 (dd, J = 8.0 Hz, 1.2 Hz, 1H), 7.46 (d , J = 3.2 Hz, 1H), 7.32-7.28 (m, 2H), 7.21 (td, J = 7.6 Hz, 1.2 Hz, 1H), 7.10-7.07 (m, 2H), 6.69 (d, J = 2.8 Hz, 1H), 6.66 (dd, J = 7.6 Hz, 1.2 Hz, 1H), 5.52 (s, 2H), 4.11 (s, 2H), 3.55 (t, J = 5.6 Hz, 2H), 2.75 (t, J = 5.6 Hz, 2H). MS (ESI) m/z 315.1[C₁₈H₁₉ClN₂O + H]⁺. |
| 13 | A | 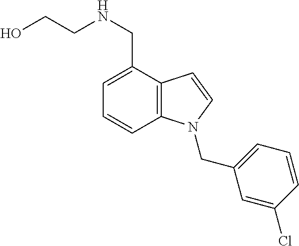 [SMILES: OCCNCc1cccc2c1ccn2Cc1cccc(Cl)c1] | ¹H NMR (400 MHz, DMSO-d₆) δ (ppm): 8.27 (s, 1H; HCOOH), 7.54 (d, J = 3.2 Hz, 1H), 7.39 (dd, J = 6.4 Hz, 2.4 Hz, 1H), 7.35-7.29 (m, 2H), 7.25 (s, 1H), 7.16-7.14 (m, 1H), 7.12-7.07 (m, 2H), 6.67 (d, J = 2.8 Hz, 1H), 5.45 (s, 2H), 4.10 (s, 2H), 3.54 (t, J = 5.6 Hz, 2H), 2.75 (t, J = 5.6 Hz, 2H). MS (ESI) m/z 315.1[C₁₈H₁₉ClN₂O + H]⁺. |
| 14 | A | 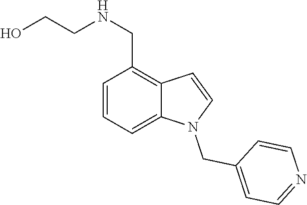 [SMILES: OCCNCc1cccc2c1ccn2Cc1ccncc1] | ¹H NMR (400 MHz, DMSO-d₆) δ (ppm): 8.47 (dd, J = 4.8, 1.6 Hz, 2H), 8.29 (s, 1H; HCOOH), 7.54 (d, J = 3.2 Hz, 1H), 7.33 (dd, J = 6.8, 1.6 Hz, 1H), 7.12-7.06 (m, 4H), 6.71 (d, J = 2.8 Hz, 1H), 5.50 (s, 2H), 4.14 (s, 2H), 3.56 (t, J = 5.6 Hz, 2H), 2.78 (t, J = 5.6 Hz, 2H). MS (ESI) m/z 282.2 [C₁₇H₁₉N₃O + H]⁺. |
| 15 | A | 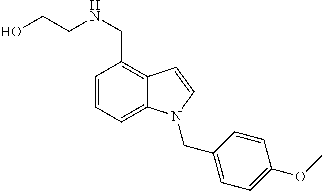 [SMILES: COc1ccc(Cn2ccc3c(CNCCO)cccc32)cc1] | ¹H NMR (400 MHz, DMSO-d₆) δ (ppm): 8.29 (s, 1H; HCOOH), 7.50 (d, J = 3.2 Hz, 1H), 7.42-7.40 (m, 1H), 7.17 (d, J = 8.8 Hz, 2H), 7.09- 7.06 (m, 2H), 6.85 (d, J = 8.8 Hz, 2H), 6.63 (d, J = 3.2 Hz, 1H), 5.33 (s, 2H), 4.11 (s, 2H), 3.69 (s, 3H), 3.55 (t, J = 5.6 Hz, 2H), 2.75 (t, J = 5.6 Hz, 2H). MS (ESI) m/z 311.1 [C₁₉H₂N₂O₂ + H]⁺. |
| 16 | A | 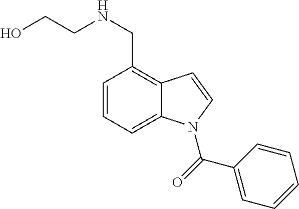 [SMILES: O=C(c1ccccc1)n1ccc2c(CNCCO)cccc21] | ¹H NMR (400 MHz, DMSO-d₆) δ (ppm): 8.24-8.21 (m, 1H), 8.16 (s, 1H; HCOOH), 7.77-7.75 (m, 2H), 7.73-7.69 (m, 1H), 7.64-7.60 (m, 2H), 7.44 (d, J = 4.0 Hz, 1H), 7.39- 7.37 (m, 2H), 6.98 (d, J = 3.6 Hz, 1H), 4.17 (s, 2H), 3.56 (t, J = 6.0 Hz, 2H), 2.77 (t→ br. s, 2H). MS (ESI) m/z 295.1[C₁₈H₁₈N₂O₂ + H]⁺. |
| 17 | A | 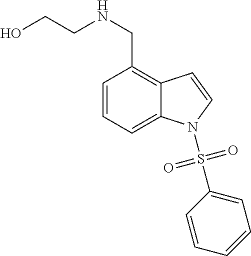 [SMILES: O=S(=O)(c1ccccc1)n1ccc2c(CNCCO)cccc21] | ¹H NMR (400 MHz, DMSO-d₆) δ (ppm): 8.25 (s, 1H; HCOOH), 8.00-7.97 (m, 2H), 7.86 (d, J = 7.6 Hz, 1H), 7.83 (d, J = 3.6 Hz, 1H), 7.71-7.69 (m, 1H), 7.61-7.57 (m, 2H), 7.34-7.28 (m, 2H), 7.03 (dd, J = 3.6 Hz, 0.8 Hz, 1H), 4.01 (s, 2H), 3.49 (t, J = 5.6 Hz, 2H), 2.66 (t, J = 4.6 Hz, 2H). MS (ESI) m/z 331.1[C₁₇H₁₈N₂O₃S + H]⁺. |
| 18 | A | 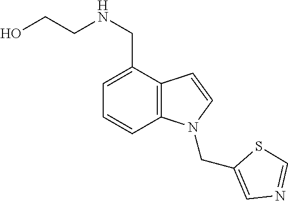 [SMILES: OCCNCc1cccc2c1ccn2Cc1cncs1] | ¹H NMR (400 MHz, DMSO-d₆) δ (ppm): 8.95 (d, J = 0.8 Hz, 1H), 8.26 (s, 1H; HCOOH), 7.95 (s, 1H), 7.52-7.49 (m, 2H), 7.15-7.07 (m, 2H), 6.63 (d, J = 3.2 Hz, 1H), 5.71 (s, 2H), 4.07 (s, 2H), 3.52 (t, J = 5.6 Hz, 2H), 2.72 (t, J = 5.6 Hz, 2H). MS (ESI) m/z 288.1 [C₁₅H₁₇N₃OS + H]⁺. |
| 19 | A | 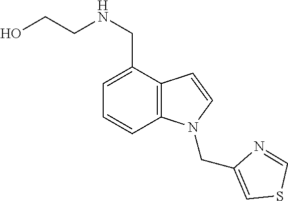 [SMILES: OCCNCc1cccc2c1ccn2Cc1cscn1] | ¹H NMR (400 MHz, DMSO-d₆) δ (ppm): 9.04 (s, 1H), 7.48 (d, J = 2.0 Hz, 1H), 7.43 (d, J = 3.2 Hz, 1H), 7.41 (d, J = 8.4 Hz, 1H), 7.07- 6.98 (m, 2H), 6.56 (d, J = 3.2 Hz, 1H), 5.51 (s, 2H), 4.45 (br. s, 1H), 3.94 (s, 2H), 3.47 (t, J = 5.6 Hz, 2H), 2.61 (t, J = 5.6 Hz, 2H). MS (ESI) m/z 288.1[C₁₅H₁₇N₃OS + H]⁺. |
| 20 | A | 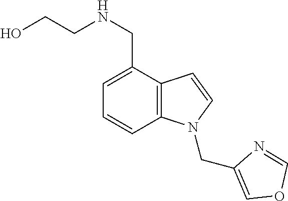 [SMILES: OCCNCc1cccc2c1ccn2Cc1cocn1] | ¹H NMR (400 MHz, DMSO-d₆) δ (ppm): 8.30 (d, J = 0.8 Hz, 1H), 8.25 (s, 1H; HCOOH), 8.08 (d, J = 0.4 Hz, 1H), 7.47 (d, J = 8.0 Hz, 1H), 7.41 (d, J = 3.2 Hz, 1H), 7.12- 7.04 (m, 2H), 6.58 (dd, J = 3.2 Hz, 0.4 Hz, 1H), 5.30 (s, 2H), 4.04 (s, 2H), 3.51 (t, J = 5.6 Hz, 2H), 2.69 (t, J = 5.6 Hz, 2H). MS (ESI) m/z 272.1[C₁₅H₁₇N₃O₂ + H]⁺. |
| 21 | A | 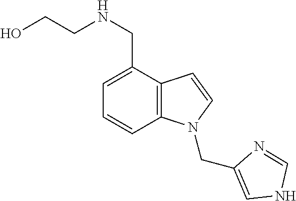 [SMILES: OCCNCc1cccc2c1ccn2Cc1c[nH]cn1] | ¹H NMR (400 MHz, DMSO-d₆) δ (ppm): 11.91 (br., 1H), 8.21 (s, 1H; HCOOH), 7.55 (s, 1H), 7.51 (d, J = 8.0 Hz, 1H), 7.39 (d, J = 3.2 Hz, 1H), 7.11-7.03 (m, 3H), 6.55 (d, J = 3.2 Hz, 1H), 5.24 (s, 2H), 4.08 (s, 2H), 3.53 (t, J = 5.6 Hz, 2H), 2.73 (t, J = 5.6 Hz, 2H). MS (ESI) m/z 271.2[C₁₅H₁₈N₄O + H]⁺. |
| 22 | A | 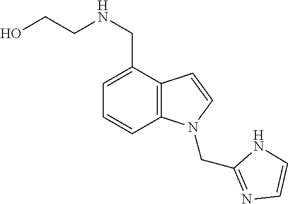 [SMILES: OCCNCc1cccc2c1ccn2Cc1ncc[nH]1] | ¹H NMR (400 MHz, DMSO-d₆) δ (ppm): 8.24 (s, 1H; HCOOH), 7.48 (d, J = 8.0 Hz, 1H), 7.39 (d, J = 3.2 Hz, 1H), 7.12-7.05 (m, 2H), 6.94 (s, 2H), 6.59 (d, J = 2.8 Hz, 1H), 5.35 (s, 2H), 4.07 (s, 2H), 3.52 (t, J = 5.6 Hz, 2H), 2.71 (t, J = 5.6 Hz, 2H). MS (ESI) m/z 271.2 [C₁₅H₁₈N₄O + H]⁺. |
| 24 | A | 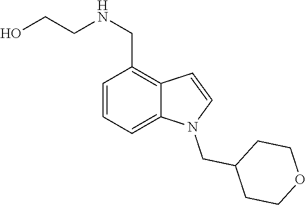 [SMILES: OCCNCc1cccc2c1ccn2CC1CCOCC1] | ¹H NMR (400 MHz, DMSO-d₆) δ (ppm): 8.31 (br. s, 1H; HCOOH), 7.46 (d, J = 7.6 Hz, 1H), 7.37 (d, J = 2.8 Hz, 1H), 7.14-7.08 (m, 2H), 6.59 (d, J = 3.2 Hz, 1H), 4.12 (s, 2H), 4.07 (d, J = 6.8 Hz, 2H), 3.82- 3.79 (m, 2H), 3.55 (t, J = 5.6 Hz, 2H), 3.19 (t, J = 10.4 Hz, 2H), 2.76 (t, J = 5.6 Hz, 2H), 2.03 (br. s, 1H), 1.37-1.25 (m, 4H). MS (ESI) m/z 289.1[C₁₇H₂₄N₂O₂ + H]⁺. |
| 25 | B | 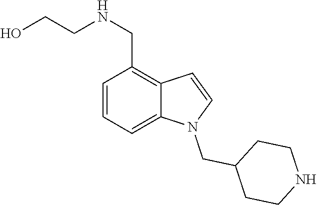 [SMILES: OCCNCc1cccc2c1ccn2CC1CCNCC1] | ¹H NMR (400 MHz, DMSO-d₆) δ (ppm): 8.32 (s, 2H; 2HCOOH), 7.38 (d, J = 8.0 Hz, 1H), 7.34 (d, J = 3.2 Hz, 1H), 7.09 (t, J = 7.6 Hz, 1H), 7.02 (d, J = 7.2 Hz, 1H), 6.55 (d, J = 2.8 Hz, 1H), 4.08 (d, J = 6.8 Hz, 2H), 4.00 (s, 2H), 3.50 (t, J = 5.6 Hz, 2H), 3.15-3.12 (m, 2H), 2.66 (m, 4H), 2.02 (br. s, 1H), 1.58-1.55 (m, 2H), 1.36-1.28 (m, 2H). MS (ESI) m/z 288.1 [C₁₇H₂₅N₃O + H]⁺. |
| 26 | A | 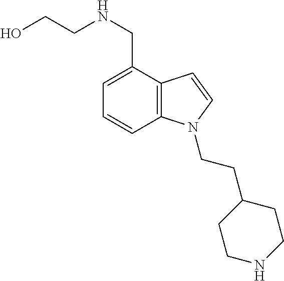 [SMILES: OCCNCc1cccc2c1ccn2CCC1CCNCC1] | ¹H NMR (400 MHz, DMSO-d₆) δ (ppm): 7.36 (d, J = 3.2 Hz, 1H), 7.33 (d, J = 8.0 Hz, 1H), 7.10 (t, J = 8.0 Hz, 1H), 7.02 (d, J = 7.2 Hz, 1H), 6.54 (d, J = 2.8 Hz, 1H), 4.50 (br, 1H), 4.22 (t, J = 7.2 Hz, 2H), 3.99 (s, 2H), 3.50 (t, J = 6.0 Hz, 2H), 3.00 (d, J = 12.0 Hz, 2H), 2.65 (t, J = 5.6 Hz, 2H), 2.52-2.48 (m, 2H), 1.72-1.67 (m, 4H), 1.36-1.34 (m, 1H), 1.19-1.13 (m, 2H). MS (ESI) m/z 302.2 [C₁₈H₂₇N₃O + H]⁺. |
| 27 | A | 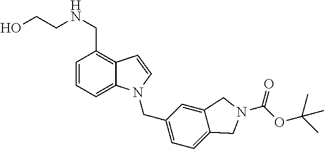 [SMILES: CC(C)(C)OC(=O)N1Cc2ccc(Cn3ccc4c(CNCCO)cccc43)cc2C1] | ¹H NMR (400MHz, DMSO-d₆) δ (ppm): 8.22 (s, 1H; HCOOH), 7.50 (t, J = 3.2 Hz, 1H), 7.36 (d, J = 7.2 Hz, 1H), 7.26-7.18 (m, 2H), 7.14-7.11 (m, 1H), 7.09-7.03 (m, 2H), 6.62 (d, J = 2.8 Hz, 1H), 5.41 (s, 2H), 4.52 2H), 4.50 (s, 2H), 4.05 (s, 2H), 3.52 (t, J = 5.6 Hz, 2H), 2.71 (t, J = 5.6 Hz, 2H), |
| | | | 1.43 (s, 9H). MS (ESI) m/z 422.1 |
| | | | [C₂₅H₃₁N₃O₃ + H]⁺. |
| 29 | A | 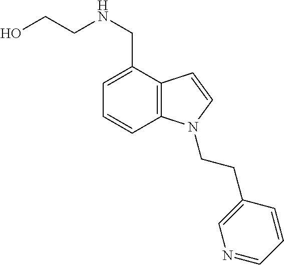 [SMILES: OCCNCc1cccc2c1ccn2CCc1cccnc1] | ¹H NMR (400 MHz, DMSO-d₆) δ (ppm): 8.40 (dd, J = 4.8 Hz, 1.2 Hz, 1H), 8.36 (d, J = 1.6 Hz, 1H), 8.31 (br. s, 1H; HCOOH), 7.61 (d, J = 8.0 Hz 1H), 7.47 (d, J = 7.2 Hz, 1H), 7.33 (d, J = 3.2 Hz, 1H), 7.28 (dd, J = 7.6 Hz, 4.8 Hz, 1H), 7.15- 7.10 (m, 2H), 6.58 (d, J = 3.2 Hz, 1H), 4.47 (t, J = 7.2 Hz, 2H), 4.14 (s, 2H), 3.57 (t, J = 5.6 Hz, 2H), 3.11 (t, J = 7.2 Hz, 2H), 2.78 (t, J = 5.6 Hz, 2H). MS (ESI) m/z 296.1 [C₁₈H₂₁N₃O + H]⁺. |
| 31 | B | 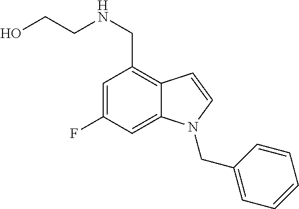 [SMILES: OCCNCc1cc(F)cc2c1ccn2Cc1ccccc1] | ¹H NMR (400 MHz, DMSO-d₆) δ (ppm): 8.23 (br. s, 1H; HCOOH), 7.50 (d, J = 3.2 Hz, 1H), 7.33-7.29 (m, 2H), 7.26-7.20 (m, 4H), 6.96 (dd, J = 10.4, 2.0 Hz, 1H), 6.62 (d, J = 3.2 Hz, 1H), 5.39 (s, 2H), 4.05 (s, 2H), 3.52 (t, J = 5.6 Hz, 2H), 2.70 (t, J = 5.6 Hz, 2H). MS (ESI) m/z 299.2[C₁₈H₁₉FN₂O + H]⁺. |
| 34 | B | 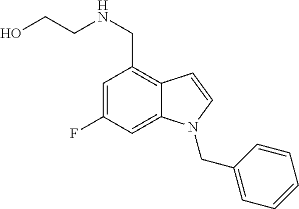 [SMILES: OCCNCc1cc(F)cc2c1ccn2Cc1ccccc1] | ¹H NMR (400 MHz, DMSO-d₆) δ (ppm): 8.25 (s, 1H; HCOOH), 7.55 (d, J = 3.2 Hz, 1H), 7.50 (s, 1H), 7.33-7.29 (m, 2H), 7.27-7.24 (m, 1H), 7.20-7.18 (m, 2H), 7.11 (s, 1H), 6.65 (d, J = 2.8 Hz, 1H), 5.43 (s, 2H), 4.06 (s, 2H), 3.52 (t, J = 5.6 Hz, 2H), 2.70 (t, J = 5.6 Hz, 2H). MS (ESI) m/z 315.1 [C₁₈H₁₉ClN₂O + H]⁺. |
| 35 | B | 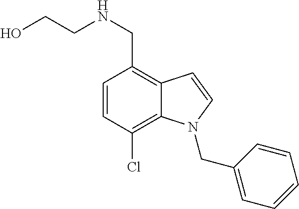 [SMILES: OCCNCc1ccc(Cl)c2c1ccn2Cc1ccccc1] | ¹H NMR (400 MHz, DMSO-d₆) δ (ppm): 8.26 (s, 1H; HCOOH), 7.59 (d, J = 3.2 Hz, 1H), 7.30-7.27 (m, 2H), 7.24-7.22 (m, 1H), 7.10-7.07 (m, 2H), 6.99-6.96 (m, 2H), 6.77 (d, J = 3.2 Hz, 1H), 5.78 (s, 2H), 4.09 (s, 2H), 3.54 (t, J = 6.0 Hz, 2H), 2.73 (t, J = 6.0 Hz, 2H). MS (ESI) m/z 315.2[C₁₈H₁₉ClN₂O + H]⁺. |
| 36 | B | 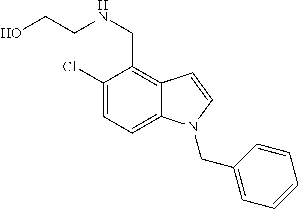 [SMILES: OCCNCc1c(Cl)ccc2c1ccn2Cc1ccccc1] | ¹H NMR (400 MHz, DMSO-d₆) δ (ppm): 8.19 (s, 1H; HCOOH), 7.59 (d, J = 3.2 Hz, 1H), 7.41 (d, J = 8.8 Hz, 1H), 7.32-7.29 (m, 2H), 7.26- 7.24 (m, 1H), 7.20-7.18 (m, 2H), 7.12 (d, J = 8.4 Hz, 1H), 6.70 (d, J = 2.8 Hz, 1H), 5.43 (s, 2H), 4.10 (s, 2H), 3.47 (t, J = 5.6 Hz, 2H), 2.66 (t, J = 5.6 Hz, 2H). MS (ESI) m/z 315.2[C₁₈H₁₉ClN₂O +H]⁺. |
| 37 | B | 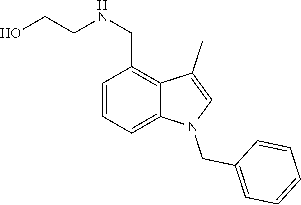 [SMILES: Cc1cn(Cc2ccccc2)c2cccc(CNCCO)c12] | ¹H NMR (400 MHz, DMSO-d₆) δ ppm: 8.28 (br. s, 1H; HCOOH), 7.34 (d, J = 8.0 Hz, 1H), 7.31-7.28 (m, 2H), 7.24-7.21 (m, 2H), 7.18 (d, J = 8.0 Hz, 2H), 7.05-6.98 (m, 2H), 5.33 (s, 2H), (s, 2H), 4.19 (s, 2H), 3.57 (t, J = 5.6 Hz, 2H), 2.81 (t, J = 5.6 Hz, 2H), 2.45 (s, 3H). MS (ESI) m/z 295.2[C₁₉H₂₂N₂O + H]⁺. |
| 38 | A | 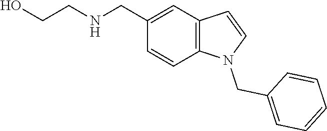 [SMILES: OCCNCc1ccc2c(ccn2Cc2ccccc2)c1] | ¹H NMR (400 MHz, DMSO-d₆) δ (ppm): 8.32 (s, 1H; HCOOH), 7.57 (s, 1H), 7.51 (d, J = 3.2 Hz, 1H), 7.42 (d, J = 8.8 Hz, 1H), 7.32-7.28 (m, 2H), 7.25-7.23 (m, 1H), 7.21- 7.17 (m, 2H), 7.14 (dd, J = 8.8 Hz, 1.6 Hz, 1H), 6.46 (d, J = 2.8 Hz, 1H), 5.41 (s, 2H), 3.93 (s, 2H), |
| | | | 3.53 (t, J = 5.6 Hz, 2H), 2.72 (t, J = |
| | | | 5.6 Hz, 2H). MS (ESI) m/z 281.2 |
| | | | [C₁₈H₂₀N₂O + H]⁺. |
| 39 | E | 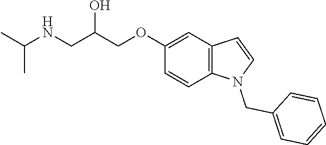 [SMILES: CC(C)NCC(O)COc1ccc2c(ccn2Cc2ccccc2)c1] | ¹H NMR (400 MHz, DMSO-d₆) δ (ppm): 8.32 (s, 1H; HCOOH), 7.45 (d, J = 3.2 Hz, 1H), 7.32-7.27 (m, 3H), 7.24-7.21 (m, 1H), 7.17-7.15 (m, 2H), 7.07 (d, J = 2.4 Hz, 1H), 6.75 (dd, J = 9.2 Hz, 2.4 Hz, 1H), 6.38 (d, J = 2.4 Hz, 1H), 5.37 (s, 2H), 4.01-3.97 (m, 1H), 3.92-3.90 (m, 2H) 3.03-2.97 (m, 1H), 2.90 |
| | | | (dd, J = 12.0 Hz, 3.6 Hz, 1H), |
| | | | 2.77-2.72 (m, 1H), (m, 1.11-1.09 |
| | | | (m, 6H). MS (ESI) m/z 339.1 |
| | | | [C₂₁H₂₆N₂O₂ + H]⁺. |
| 40 | F | 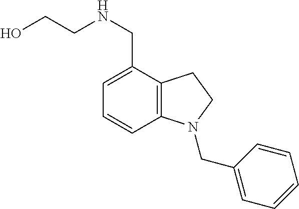 [SMILES: OCCNCc1cccc2c1CCN2Cc1ccccc1] | ¹H NMR (400 MHz, DMSO-d₆) δ (ppm): 8.26 (s, 1H; HCOOH), 7.35-7.34 (m, 4H), 7.28-7.25 (m, 1H), 6.97 (t, J = 7.6 Hz, 1H), 6.64 (d, J = 7.6 Hz, 1H), 6.51 (d, J = 8.0 Hz, 1H), 4.26 (s, 2H), 3.72 (s, 2H), 3.53 (t, J = 5.6 Hz, 2H), 3.27 (t, J = 8.4 Hz, 2H), 2.92 (t, J = 8.4 Hz, 2H), 2.71 (t, J = 5.6 Hz, 2H). MS (ESI) m/z 283.2[C₁₈H₂₂N₂O + H]⁺. |
| 42 | G | 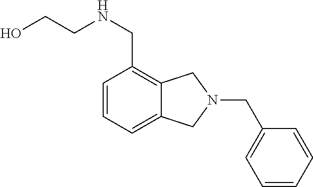 [SMILES: OCCNCc1cccc2c1CN(Cc1ccccc1)C2] | ¹H NMR (400 MHz, DMSO-d₆) δ (ppm): 7.40-7.33 (m, 4H), 7.29- 7.25 (m, 1H), 7.16-7.13 (m, 2H), 7.09-7.07 (m, 1H), 4.44 (t, J = 5.2 Hz, 1H), 3.88 (s, 2H), 3.87 (s, 2H), 3.83 (s, 2H), 3.61 (s, 2H), 3.43 (q, J = 5.2 Hz, 2H), 2.53 (t, J = 6.0 Hz, 2H). MS (ESI) m/z 283.1[C₁₈H₂₂N₂O + H]⁺. |
| 43 | G | 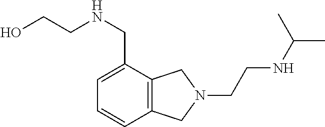 [SMILES: CC(C)NCCN1Cc2cccc(CNCCO)c2C1] | ¹H NMR (400 MHz, DMSO-d₆) δ (ppm): 7.15-7.14 (m, 2H), 7.10- 7.08 (m, 1H), 4.45 (br. s, 1H), 3.89 (s, 2H), 3.85 (s, 2H), 3.64 (s, 2H), 3.45 (br. t, 2H), 2.80-2.76 (m, 3H), 2.73-2.71 (m, 2H), 2.56-2.53 (m, 2H), 1.01 (d, J = 6.0 Hz, 6H). MS (ESI) m/z 278.2[C₁₆H₂₇N₃O + H]⁺. |
| 44 | B | 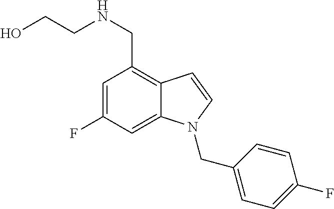 [SMILES: OCCNCc1cc(F)cc2c1ccn2Cc1ccc(F)cc1] | ¹H NMR (400 MHz, DMSO-d₆) δ (ppm): 8.23 (br. s, 1H; HCOOH) 7.48 (d, J = 3.2 Hz, 1H), 7.29-7.23 (m, 3H), 7.16-7.12 (m, 2H), 6.94 (dd, J = 10.4 Hz, 2.0 Hz, 1H), 6.60 (d, J = 3.2 Hz, 1H), 5.37 (s, 2H), 4.01 (s, 2H), 3.51 (t, J = 5.6 Hz, 2H), 2.67 (t, J = 5.6 Hz, 2H). MS (ESI) m/z 317.1[C₁₈H₁₈F₂N₂O + H]⁺. |
| 45 | B | 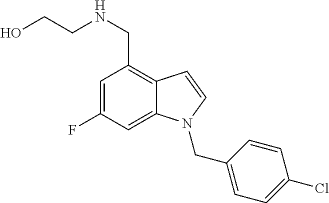 [SMILES: OCCNCc1cc(F)cc2c1ccn2Cc1ccc(Cl)cc1] | ¹H NMR (400 MHz, DMSO-d₆) δ (ppm): 8.22 (s, 1H; HCOOH), 7.50 (d, J = 3.2 Hz, 1H), 7.38 (dd, J = 6.4, 2.0 Hz, 2H), 7.26-7.22 (m, 3H), 6.96 (dd, J = 10.8, 2.4 Hz, 1H), 6.62 (dd, J = 3.2, 0.8 Hz, 1H), 5.39 (s, 2H), 4.04 (s, 2H), 3.52 (t, J = 5.6 Hz, 2H), 2.69 (t, J = 5.6 Hz, 2H). MS (ESI) m/z 333.1 [C₁₈H₁₈ClFN₂O + H]⁺. |
| 46 | H | 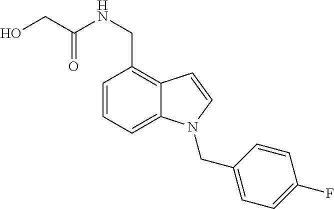 [SMILES: O=C(CO)NCc1cccc2c1ccn2Cc1ccc(F)cc1] | ¹H NMR (400 MHz, DMSO-d₆) δ (ppm): 8.09 (t, J = 6.0 Hz, 1H), 7.49 (d, J = 3.2 Hz, 1H), 7.34 (d, J = 8.4 Hz, 1H), 7.25-7.22 (m, 2H), 7.12 (t, J = 8.8 Hz, 2H), 7.04 (t, J = 8.0 Hz, 1H), 6.91 (d, J = 6.8 Hz, 1H), 6.61 (d, J = 2.8 Hz, 1H), 5.47 (t, J = 6.0 Hz, 1H), 5.40 (s, 2H), 4.55 (t, J = 6.0 Hz, 2H), 3.85 (d, J = 6.0 Hz, 2H). MS (ESI) m/z 313.2[C₁₈H₁₇FN₂O₂ +H]⁺. |
| 47 | A | 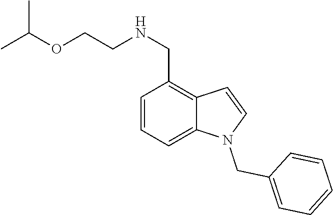 [SMILES: CC(C)OCCNCc1cccc2c1ccn2Cc1ccccc1] | ¹H NMR (400 MHz, DMSO-d₆) δ (ppm): 8.26 (s, 1H; HCOOH), 7.51 (d, J = 3.2 Hz, 1H), 7.37 (d, J = 7.6 Hz, 1H), 7.31-7.28 (m, 2H), 7.25- 7.18 (m, 3H), 7.09-7.03 (m, 2H), 6.63 (d, J = 2.8 Hz, 1H), 5.42 (s, 2H), 4.07 (s, 2H), 3.55-3.47 (m, 3H), 2.78 (t, J = 5.6 Hz, 2H), 1.07 (d, J = 6.0 Hz, 6H). MS (ESI) m/z 323.3[C₂₁H₂₆N₂O + H]⁺. |
| 48 | B | 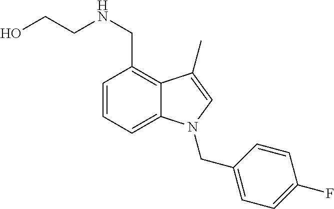 [SMILES: Cc1cn(Cc2ccc(F)cc2)c2cccc(CNCCO)c12] | ¹H NMR (400 MHz, DMSO-d₆) δ (ppm): 8.24 (s, 1H; HCOOH), 7.32 (d, J = 8.0 Hz, 1H), 7.24-7.21 (m, 3H), 7.14-7.10 (m, 2H), 7.02 (t, J = 8.0 Hz, 1H), 6.95 (d, J = 6.8 Hz, 1H), 5.31 (s, 2H), 4.13 (s, 2H), 3.54 (t, J = 5.6 Hz, 2H), 2.76 (t, J = 5.6 Hz, 2H), 2.44 (s, 3H). MS (ESI) m/z 313.2[C₁₉H₂₁FN₂O + H]⁺. |
| 49 | B | 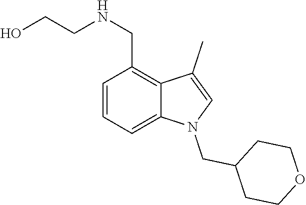 [SMILES: Cc1cn(CC2CCOCC2)c2cccc(CNCCO)c12] | ¹H NMR (400 MHz, DMSO-d₆) δ (ppm): 8.28 (br. s, 1H; HCOOH), 7.38 (d, J = 8.0 Hz, 1H), 7.10 (s, 1H), 7.06 (t, J = 8.0 Hz, 1H), 6.98 (d, J = 6.8 Hz, 1H), 4.20 (s, 2H), 3.98 (d, J = 7.2 Hz, 2H), 3.80 (dd, J = 11.6 Hz, 2.4 Hz, 2H), 3.57 (t, J = 5.2 Hz, 2H), 3.18 (t, J = 10.4 Hz, 2H), 2.82 (t, J = 5.6 Hz, 2H), 2.43 (s, 3H), 2.00-1.95 (m, 1H), 1.37-1.35 (m, 2H), 1.30-1.20 (m, 2H). MS (ESI) m/z 303.2 [C₁₈H₂₆N₂O₂ + H]⁺. |
| 50 | B | 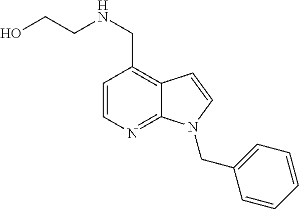 [SMILES: OCCNCc1ccnc2c1ccn2Cc1ccccc1] | ¹H NMR (400 MHz, DMSO-d₆) δ (ppm): 8.23-8.22 (m, 2H), 7.59 (d, J = 3.6 Hz, 1H), 7.32-7.28 (m, 2H), 7.25-7.21 (m, 3H), 7.15 (d, J = 4.8 Hz, 1H), 6.64 (d, J = 3.6 Hz, 1H), 5.48 (s, 2H), 4.09 (s, 2H), 3.52 (t, J = 5.6 Hz, 2H), 2.69 (t, J = 5.6 Hz, 2H). MS (ESI) m/z 282.2 [C₁₇H₁₉N₃O + H]⁺. |
| 51 | B | 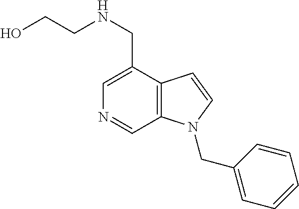 [SMILES: OCCNCc1cncc2c1ccn2Cc1ccccc1] | ¹H NMR (400 MHz, DMSO-d₆) δ (ppm): 8.71 (s, 1H), 8.05 (s, 1H), 7.72 (d, J = 3.2 Hz, 1H), 7.34-7.30 (m, 2H), 7.27-7.24 (m, 3H), 6.66 (d, J = 3.2 Hz, 1H), 5.52 (s, 2H), 4.45 (t, 1H), 3.96 (s, 2H), 3.49- 3.45 (m, 2H), 2.61 (t, J = 6.0 Hz, 2H). MS (ESI) m/z 282.1 [C₁₇H₁₉N₃O + H]⁺. |
| 52 | H | 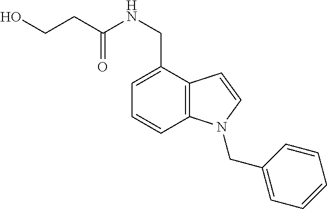 [SMILES: O=C(CCO)NCc1cccc2c1ccn2Cc1ccccc1] | ¹H NMR (400 MHz, DMSO-d₆) δ (ppm): 8.24 (t, J = 5.6 Hz, 1H), 7.49 (d, J = 3.2 Hz, 1H), 7.35-7.27 (m, 3H), 7.24-7.21 (m, 1H), 7.19- 7.17 (m, 2H), 7.03 (t, J = 8.0 Hz, 1H), 6.91 (d, J = 7.2 Hz, 1H), 6.55 (d, J = 2.8 Hz, 1H), 5.42 (s, 2H), 4.57 (t, J = 5.2 Hz, 1H), 4.51 (d, J = 5.6 Hz, 2H), 3.66-3.62 (m, 2H), 2.30 (t, J = 6.4 Hz, 2H). MS (ESI) m/z 309.1[C₁₉H2₀N₂O₂ + H]⁺. |
| 53 | H | 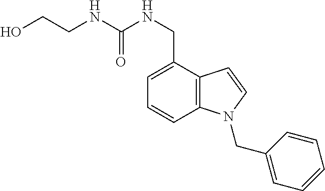 [SMILES: O=C(NCCO)NCc1cccc2c1ccn2Cc1ccccc1] | ¹H NMR (400 MHz, DMSO-d₆) δ (ppm): 7.49 (d, J = 7.2 Hz, 1H), 7.33-7.27 (m, 3H), 7.24-7.21 (m, 1H), 7.18-7.16 (m, 2H), 7.03 (t, J = 8.0 Hz, 1H), 6.89 (d, J = 7.2 Hz, 1H), 6.56 (d, J = 3.2 Hz, 1H), 6.36 (d, J = 5.6 Hz, 1H), 5.93 (t, J = 5.6 Hz, 1H), 5.41 (s, 2H), 4.65 (t, J = 5.2 Hz, 1H), 4.45 (d, J = 6.0 Hz, 2H), 3.40-3.36 (m, 2H), 3.11-3.07 (m, 2H). MS (ESI) m/z 324.2 [C₁₉H₂₁N₃O₂ + H]⁺. |
| 54 | B | 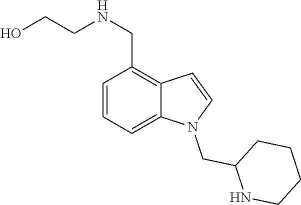 [SMILES: OCCNCc1cccc2c1ccn2CC1CCCCN1] | ¹H NMR (400 MHz, DMSO-d₆) δ (ppm): 7.36 (d, J = 8.0 Hz, 1H), 7.29 (d, J = 3.2 Hz, 1H), 7.07 (t, J = 7.4 Hz, 1H), 6.99 (d, J = 6.8 Hz, 1H), 6.51 (d, J = 2.4 Hz, 1H), 4.45 (br. s, 1H), 4.06-4.03 (m, 2H), 3.95 (s, 2H), 3.48 (t, J = 5.6 Hz, 2H), 2.91-2.88 (m, 1H), 2.83-2.80 (m, 1H), 2.63 (t, J = 5.6 Hz, 2H), 2.41-2.38 (m, 1H), 1.70-1.67 (m, 1H), 1.47-1.42 (m, 2H), 1.28-1.17 (m, 2H), 1.08-1.03 (m, 1H). MS (ESI) m/z 288.1[C₁₇H₂₅N₃O + H]⁺. |
| 77 | D | 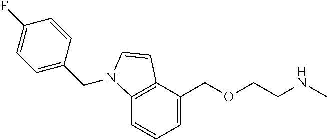 [SMILES: CNCCOCc1cccc2c1ccn2Cc1ccc(F)cc1] | ¹H NMR (400 MHz, DMSO-d₆) δ (ppm): 8.30 (s, 1H; HCOOH), 7.53 (d, J = 3.2 Hz, 1H), 7.42 (d, J = 8.4 Hz, 1H), 7.27-7.24 (m, 2H), 7.16-7.02 (m, 4H), 6.59 (d, J = 2.8 Hz, 1H), 5.42 (s, 2H), 4.76 (s, 2H), 3.62 (t, J = 5.2 Hz, 2H), 2.93 (t, J = 5.2 Hz, 2H), 2.43 (s, 3H). MS (ESI) m/z 313.2 |
| | | | [C₁₉H₂₁FN₂O + H]⁺. |
| 78 | E | 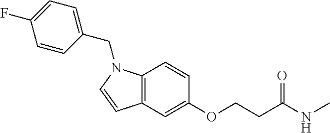 [SMILES: CNC(=O)CCOc1ccc2c(ccn2Cc2ccc(F)cc2)c1] | ¹H NMR (400 MHz, DMSO-d₆) δ ppm: 7.88 (d, J = 3.6 Hz, 1H), 7.44 (d, J = 3.2 Hz, 1H), 7.31 (d, J = 8.8 Hz, 1H), 7.23-7.19 (m, 2H), 7.15- 7.09 (m, 2H), 7.05 (d, J = 2.4 Hz, 1H), 6.70 (dd, J = 8.8 Hz, 2.4 Hz, 1H), 6.37 (d, J = 2.4 Hz, 1H), 5.35 (s, 2H), 4.14 (t, J = 6.4 Hz, 2H), |
| | | | 2.59 (d, J = 4.8 Hz, 3H), 2.52-2.49 |
| | | | (m, 2H). MS (ESI) m/z 327.3 |
| | | | [C₁₉H₁₉FN₂O₂ + H]⁺. |
| 79 | E | 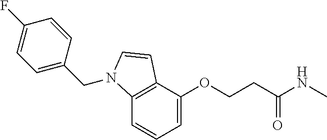 [SMILES: CNC(=O)CCOc1cccc2c1ccn2Cc1ccc(F)cc1] | ¹H NMR (400 MHz, DMSO-d₆) δ ppm: 7.92 (d, J = 3.6 Hz, 1H), 7.36 (d, J = 3.2 Hz, 1H), 7.22-7.18 (m, 2H), 7.15-7.09 (m, 2H), 7.04-6.97 (m, 2H), 6.52 (dd, J = 7.2, 0.8 Hz, 1H), 6.42 (d, J = 2.8 Hz, 1H), 5.37 (s, 2H), 4.27 (t, J = 6.4 Hz, 2H), 2.61 (d, J = 4.8 Hz, 3H), 2.58 (t, J = 6.0 Hz, 2H). MS (ESI) m/z |
| | | | 327.1[C₁₉H₁₉FN₂O₂ + H]⁺. |
| 80 | B | 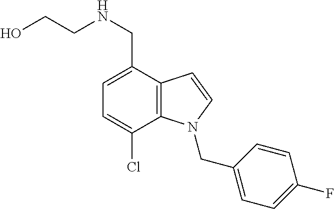 [SMILES: OCCNCc1ccc(Cl)c2c1ccn2Cc1ccc(F)cc1] | ¹H NMR (400 MHz, DMSO-d₆) δ (ppm): 8.22 (s, 1H; HCOOH), 7.58-7.57 (d, J = 3.2 Hz, 1H), 7.12- 7.02 (m, 6H), 6.75-6.74 (d, J = 3.2 Hz, 1H), 5.75 (s, 2H), 4.03 (s, 2H), 3.52 (t, J = 5.6 Hz, 2H), 2.69 (t, J = 5.6 Hz, 2H). MS (ESI) m/z 333.2 [C₁₈H₁₈ClFN₂O + H]⁺. |
| 81 | B | 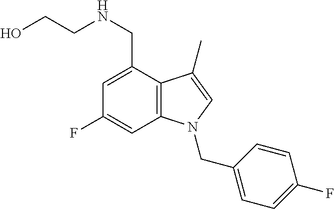 [SMILES: Cc1cn(Cc2ccc(F)cc2)c2cc(F)cc(CNCCO)c12] | ¹H NMR (400 MHz, DMSO-d₆) δ (ppm): 8.21 (s, 1H; HCOOH), 7.27-7.19 (m, 4H), 7.16-7.11 (m, 2H), 6.86 (dd, J = 10.8 Hz, 2.4 Hz, 1H), 5.27 (s, 2H), 4.11 (s, 2H), 3.52 (t, J = 5.6 Hz, 2H), 2.72 (t, J = 5.6 Hz, 2H), 2.40 (s, 3H). MS (ESI) m/z 331.1[C₁₉H₂₀F₂N₂O + H]⁺. |
| 83 | I | 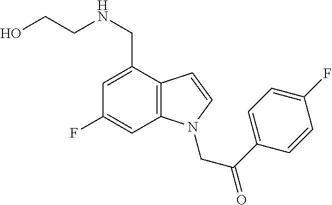 [SMILES: O=C(Cn1ccc2c(CNCCO)cc(F)cc21)c1ccc(F)cc1] | ¹H NMR (400 MHz, DMSO-d₆) δ (ppm): 8.19-8.15 (m, 2H), 7.47- 7.42 (m, 2H), 7.29 (d, J = 3.2 Hz, 1H), 7.16 (dd, J = 10.0 Hz, 2.0 Hz, 1H), 6.91 (dd, J = 10.8 Hz, 2.4 Hz, 1H), 6.58 (d, J = 3.2 Hz, 1H), 5.87 (s, 2H), 4.50 (t, J = 5.6 Hz, 1H), 3.98 (s, 2H), 3.50 (q, J = 5.6 Hz, 2H), 2.64 (t, J = 5.6 Hz, 2H). MS (ESI) m/z 345.3[C₁₉H₁₈F₂N₂O₂ + H]⁺. |
| 108 | A | 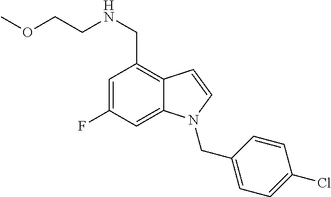 [SMILES: COCCNCc1cc(F)cc2c1ccn2Cc1ccc(Cl)cc1] | ¹H NMR (400 MHz, DMSO-d₆) δ (ppm): 8.20 (s, 1H; HCOOH), 7.49 (d, J = 3.2 Hz, 1H), 7.39-7.36 (m, 2H), 7.24-7.21 (m, 3H), 6.93 (dd, J = 10.8 Hz, 2.4 Hz, 1H), 6.61 (d, J = 3.2 Hz, 1H), 5.39 (s, 2H), 4.01 (s, 2H), 3.43 (t, J = 5.6 Hz, 2H), 3.24 (s, 3H), 2.75 (t, J = 5.6 Hz, 2H). MS (ESI) m/z 347.1 [C₁₉H₂₀ClFN₂O + H]⁺. |
| 110 | J | 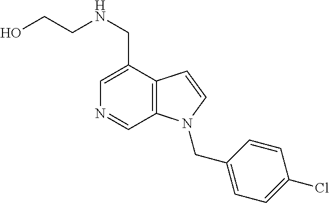 [SMILES: OCCNCc1cncc2c1ccn2Cc1ccc(Cl)cc1] | ¹H NMR (400 MHz, DMSO-d₆) δ (ppm): 8.71 (s, 1H), 8.06 (s, 1H), 7.73 (d, J = 3.2 Hz, 1H), 7.40-7.37 (m, 2H), 7.29-7.26 (m, 2H), 6.67 (dd, J = 3.2 Hz, 0.8 Hz, 1H), 5.53 (s, 2H), 4.46 (t, J = 5.6 Hz, 1H), 3.95 (s, 2H), 3.46 (q, J = 5.6 Hz, 2H), 2.59 (t, J = 5.6 Hz, 2H). MS (ESI) m/z 316.0[C₁₇H₁₈ClN₃O + H]⁺. |
| 111 | B | 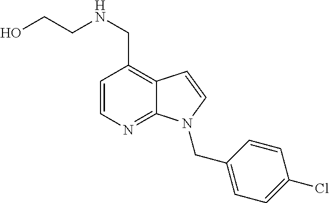 [SMILES: OCCNCc1ccnc2c1ccn2Cc1ccc(Cl)cc1] | ¹H NMR (400 MHz, DMSO-d₆) δ (ppm): 8.22 (d, J = 4.8 Hz, 1H), 8.20 (s, 1H; HCOOH), 7.61 (d, J = 3.2 Hz, 1H), 7.38-7.35 (m, 2H), 7.25-7.23 (m, 2H), 7.16 (d, J = 4.8 Hz, 1H), 6.65 (d, J = 3.6 Hz, 1H), 5.47 (s, 2H), 4.08 (s, 2H), 3.52 (t, J = 5.6 Hz, 2H), 2.67 (t, J = 5.6 Hz, 2H). MS (ESI) m/z 316.1 [C₁₇H₁₈ClN₃O + H]⁺. |
| 112 | B | 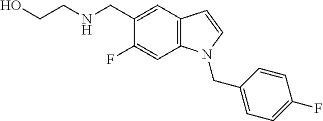 [SMILES: OCCNCc1cc2ccn(Cc3ccc(F)cc3)c2cc1F] | ¹H NMR (400 MHz, DMSO-d₆) δ (ppm): 7.53 (d, J = 7.6 Hz, 1H), 7.47 (d, J = 3.2 Hz, 1H), 7.32-7.24 (m, 3H), 7.14 (t, J = 8.8 Hz, 2H), 6.45 (d, J = 2.8 Hz, 1H), 5.36 (s, 2H), 4.45 (t, J = 5.2 Hz, 1H), 3.76 (s, 2H), 3.45 (q, J = 5.2 Hz, 2H), 2.57 (t, J = 5.6 Hz, 2H), 1.97 (br. |
| | | | s, 1H). MS (ESI) m/z 317.2 |
| | | | [C₁₈H₁₈F₂N₂O + H]⁺. |
| 113 | J | 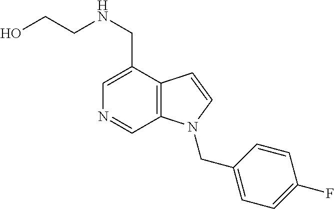 [SMILES: OCCNCc1cncc2c1ccn2Cc1ccc(F)cc1] | ¹H NMR (400 MHz, DMSO-d₆) δ (ppm): 8.74 (s, 1H), 8.05 (s, 1H), 7.73 (d, J = 3.2 Hz, 1H), 7.35-7.31 (m, 2H), 7.18-7.13 (m, 2H), 6.66 (d, J = 3.2 Hz, 1H), 5.51 (s, 2H), 4.45 (t, J = 5.2 Hz, 1H), 3.95 (s, 2H), 3.46 (q, J = 5.2 Hz, 2H), 2.59 (t, J = 5.6 Hz, 2H). MS (ESI) m/z 300.0[C₁₇H₁₈FN₃O + H]⁺. |
| 114 | J | 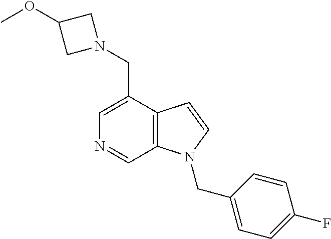 [SMILES: COC1CN(Cc2cncc3c2ccn3Cc2ccc(F)cc2)C1] | ¹H NMR (400 MHz, DMSO-d₆) δ (ppm): 8.74 (s, 1H), 7.99 (s, 1H), 7.72 (d, J = 3.2 Hz, 1H), 7.35-7.31 (m, 2H), 7.17-7.13 (m, 2H), 6.62 (d, J = 2.8 Hz, 1H), 5.50 (s, 2H), 3.96-3.93 (m, 1H), 3.80 (s, 2H), 3.47-3.44 (m, 2H), 3.13 (s, 3H), 2.88-2.85 (m, 2H). MS (ESI) m/z 326.1[C₁₉H₂₀FN₃O + H]⁺. |
| 115 | B | 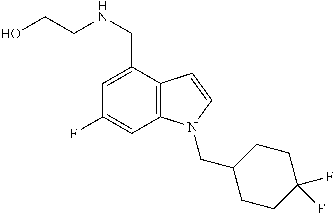 [SMILES: OCCNCc1cc(F)cc2c1ccn2CC1CCC(F)(F)CC1] | ¹H NMR (400 MHz, DMSO-d₆) δ ppm: 8.53 (br, 2H; HCl), 7.50 (dd, J = 10.4, 1.6 Hz, 1H), 7.44 (d, J = 3.2 Hz, 1H), 7.12 (dd, J = 10.4, 1.6 Hz, 1H), 6.69 (d, J = 2.8 Hz, 1H), 5.11 (s, 1H), 4.33 (s, 2H), 4.09 (d, J = 7.2 Hz, 2H), 3.65 (br. s, 2H), 2.95 (t, J = 5.2 Hz, 2H), 1.97-1.91 (m, 3H), 1.80-1.65 (m, 2H), 1.57-1.54 (m, 2H), 1.30-1.25 (m, 2H). MS (ESI) m/z 341.2 [C₁₈H₂₃F₃N₂O + H]⁺. |
| 116 | B | 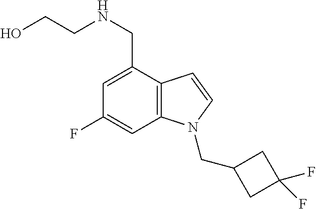 [SMILES: OCCNCc1cc(F)cc2c1ccn2CC1CC(F)(F)C1] | ¹H NMR (400 MHz, DMSO-d₆) δ ppm: 8.24 (s, 1H; HCOOH), 7.42 (d, J = 3.2 Hz, 1H), 7.32 (dd, J = 10.4, 2.0 Hz, 1H), 6.95 (dd, J = 10.8, 2.0 Hz, 1H), 6.56 (d, J = 3.2 Hz, 1H), 4.26 (d, J = 6.4 Hz, 2H), 4.02 (s, 2H), 3.51 (t, J = 5.6 Hz, 2H), 2.68 (t, J = 5.6 Hz, 2H), 2.61-2.56 (m, 3H), 2.46-2.35 (m, 2H). MS (ESI) m/z 313.4 [C₁₆H₁₉F₃N₂O + H]⁺. |
| 178 | D | 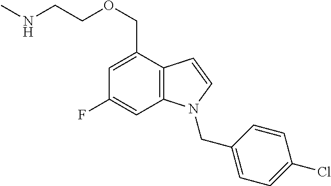 [SMILES: CNCCOCc1cc(F)cc2c1ccn2Cc1ccc(Cl)cc1] | ¹H NMR (400 MHz, DMSO-d₆) δ ppm: 8.30 (s, 1H; HCOOH), 7.52 (d, J = 3.2 Hz, 1H), 7.38 (d, J = 8.4 Hz, 2H), 7.29 (dd, J = 10.4, 2.0 Hz, 1H), 7.22 (d, J = 8.4 Hz, 2H), 6.94 (dd, J = 10.4, 2.0 Hz, 1H), 6.58 (d, J = 3.2 Hz, 1H), 5.40 (s, 2H), 4.76 (s, 2H), 3.63 (t, J = 5.2 Hz, 2H), 2.92 (t, J = 5.2 Hz, 2H), 2.42 (s, 3H). MS (ESI) m/z 347.3 [C₁₉H₂₀ClFN₂O + H]⁺. |
| 179 | D | 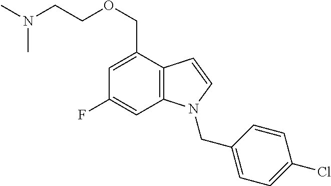 [SMILES: CN(C)CCOCc1cc(F)cc2c1ccn2Cc1ccc(Cl)cc1] | ¹H NMR (400 MHz, DMSO-d₆) δ ppm: 7.50 (d, J = 3.2 Hz, 1H), 7.37 (d, J = 8.4 Hz, 2H), 7.27 (dd, J = 10.0, 2.0 Hz, 1H), 7.21 (d, J = 8.4 Hz, 2H), 6.86 (dd, J = 10.4, 2.0 Hz, 1H), 6.56 (d, J = 2.8 Hz, 1H), 5.39 (s, 2H), 4.71 (s, 2H), 3.56 (t, J = 6.0 Hz, 2H), 2.45 (t, J = 6.0 Hz, 2H), 2.15 (s, 6H). MS (ESI) m/z 361.1[C₂₀H₂₂ClFN₂O + H]⁺. |
| 180 | D | 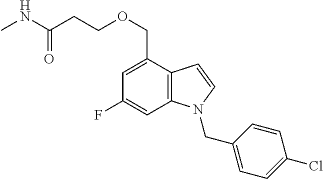 [SMILES: CNC(=O)CCOCc1cc(F)cc2c1ccn2Cc1ccc(Cl)cc1] | ¹H NMR (400 MHz, DMSO-d₆) δ ppm: 7.79 (d, J = 3.6 Hz, 1H), 7.49 (d, J = 3.2 Hz, 1H), 7.38 (d, J = 8.4 Hz, 2H), 7.26 (dd, J = 10.4, 2.0 Hz, 1H), 7.21 (d, J = 8.4 Hz, 2H), 6.85 (dd, J = 10.4, 2.0 Hz, 1H), 6.55 (d, J = 3.2 Hz, 1H), 5.39 (s, 2H), 4.69 (s, 2H), 3.68 (t, J = 6.4 Hz, 2H), 2.56 (d, J = 4.4 Hz, 3H), 2.35 (t, J = 6.4 Hz, 2H). MS (ESI) m/z 375.0[C₂₀H₂₀ClFN₂O₂ + H]⁺. |
| 181 | D | 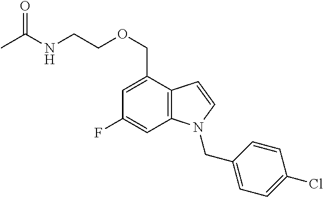 [SMILES: CC(=O)NCCOCc1cc(F)cc2c1ccn2Cc1ccc(Cl)cc1] | ¹H NMR (400 MHz, DMSO-d₆) δ ppm: 7.94 (t, 1H), 7.50 (d, J = 3.2 Hz, 1H), 7.38-7.36 (m, 2H), 7.27 (dd, J = 10.4, 2.0 Hz, 1H), 7.22 (d, J = 8.4 Hz, 2H), 6.90 (dd, J = 10.4, 2.0 Hz, 1H), 6.57 (dd, J = 3.2, 0.8 Hz, 1H), 5.39 (s, 2H), 4.72 (s, 2H), 3.47 (t, J = 5.6 Hz, 2H), 3.25 (q, J = 5.6 Hz, 2H), 1.79 (s, 3H). MS (ESI) m/z 375.3[C₂₀H₂₀ClFN₂O₂ + H]⁺. |

TABLE 5
Summary of compounds (series II) prepared according to the examples above and characterizations thereof

| Cpd | Synthesis | | NMR and Mass |
|---|---|---|---|
| No. | Route | Structure | Spectrometry |
| 84 | A | 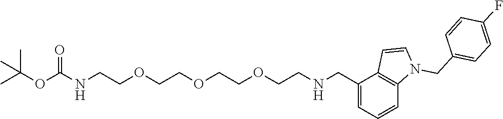 [SMILES: CC(C)(C)OC(=O)NCCOCCOCCOCCNCc1cccc2c1ccn2Cc1ccc(F)cc1] | ¹H NMR (400 MHz, DMSO-d₆-D₂O) δ (ppm): 8.36 (s, 1H; HCOOH), 7.59 (d, J = 3.2 Hz, 1H), 7.50 (dd, J = 7.2 Hz, 1.2 Hz, 1H), 7.30-7.27 (m, 2H), 7.20-7.11 (m, 4H), 6.72 (d, J = 3.2 Hz, 1 H), |
| | | | 5.42 (s, 2H), 4.34 (s, 2H), |
| | | | 3.67 (t, J = 4.8 Hz, 2H), |
| | | | 3.56 (s, 4H), 3.51-3.47 |
| | | | (m, 4H), 3.35 (t, J = 6.0 |
| | | | Hz, 2H), 3.10-3.04 (m, |
| | | | 4H), 1.36 (s, 9H). MS |
| | | | (ESI) m/z 530.4 |
| | | | [C₂₉H₄₀FN₃O₅ + H]⁺. |
| 85 | A | 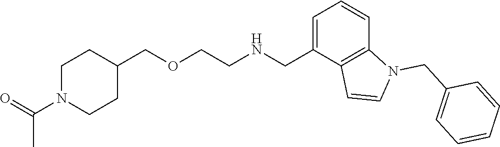 [SMILES: CC(=O)N1CCC(COCCNCc2cccc3c2ccn3Cc2ccccc2)CC1] | ¹H NMR (400 MHz, DMSO-d₆) δ (ppm): 8.21 (s, 1H; HCOOH), 7.49 (d, J = 3.2 Hz, 1H), 7.35- 7.28 (m, 3H), 7.25-7.23 (m, 1H), 7.22-7.18 (m, 2H), 7.07-7.00 (m, 2H), 6.59 (d, J = 2.8 Hz, 1H), 5.41 (s, 2H), 4.35-4.32 |
| | | | (m, 1H), 4.01 (s, 2H), |
| | | | 3.78-3.74 (m, 1H), 3.47 |
| | | | (t, J = 5.6 Hz, 2H), 3.21 |
| | | | (d, J = 6.4 Hz, 2H), 2.99- |
| | | | 2.93 (m, 1H), 2.75 (t, J = |
| | | | 5.6 Hz, 2H), 2.46-2.43 |
| | | | (m, 1H), 1.96 (s, 3H), |
| | | | 1.78-1.71 (m, 1H), 1.67- |
| | | | 1.59 (m, 2H), 1.10-0.89 |
| | | | (m, 2H). MS (ESI) m/z |
| | | | 420.3[C₂₆H₃₃N₃O₂ + H]⁺. |
| 86 | B | 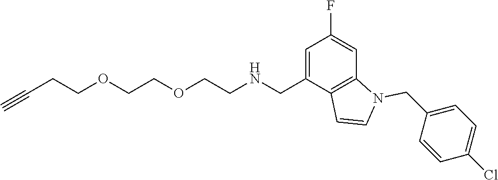 [SMILES: C#CCCOCCOCCNCc1cc(F)cc2c1ccn2Cc1ccc(Cl)cc1] | ¹H NMR (400 MHz, DMSO-d₆) δ (ppm): 7.47 (d, J = 3.6 Hz, 1H), 7.39- 7.36 (m, 2H), 7.23-7.17 (m, 3H), 6.89 (dd, J = 10.8 Hz, 2.4 Hz, 1H), 6.59 (d, J = 3.2 Hz, 1H), 5.38 (s, 2H), 3.95 (s, 2H), 3.51-3.46 (m, 8H), 2.77 (t, J = 2.8 Hz, 1H), 2.70- 2.67 (m, 2H), 2.38-2.34 |
| | | | (m, 2H), 2.19 (br, 1H). |
| | | | MS (ESI) m/z 429.1 |
| | | | [C₂₄H₂₆ClFN₂O₂ + H]⁺. |
| 87 | B | 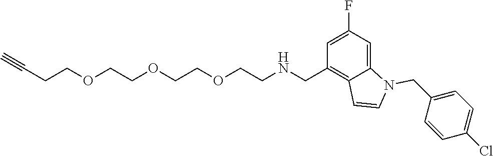 [SMILES: C#CCCOCCOCCOCCNCc1cc(F)cc2c1ccn2Cc1ccc(Cl)cc1] | ¹H NMR (400 MHz, DMSO-d₆) δ (ppm): 7.47 (d, J = 3.2 Hz, 1H), 7.39- 7.36 (m, 2H), 7.23-7.18 (m, 3H), 6.89 (dd, J = 10.8 Hz, 2.0 Hz, 1H), 6.59 (d, J = 2.8 Hz, 1H), 5.38 (s, 2H), 3.95 (s, 2H), 3.52-3.44 (m, 12H), 2.78 (t, J = 2.8 Hz, 1H), |
| | | | 2.69 (t, J = 5.6 Hz, 2H), |
| | | | 2.38-2.34 (m, 2H). MS |
| | | | (ESI) m/z 473.2 |
| | | | [C₂₆H₃₀ClFN₂O₃ + H]⁺. |
| 88 | B | 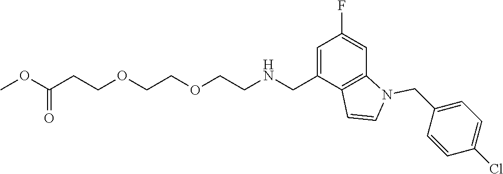 [SMILES: COC(=O)CCOCCOCCNCc1cc(F)cc2c1ccn2Cc1ccc(Cl)cc1] | ¹H NMR (400 MHz, DMSO-d₆) δ (ppm): 7.47 (d, J = 3.2 Hz, 1H), 7.39- 7.36 (m, 2H), 7.22-7.18 (m, 3H), 6.89 (dd, J = 10.8 Hz, 2.4 Hz, 1H), 6.59 (d, J = 3.2 Hz, 1H), 5.38 (s, 2H), 3.95 (s, 2H), 3.61 (t, J = 6.0 Hz, 2H), 3.57 (s, 3H), 3.49-3.46 (m, 6H), 2.68 (t, J = 5.6 |
| | | | Hz, 2H), 2.53-2.51 (m, |
| | | | 2H). MS (ESI) m/z 463.1 |
| | | | [C₂₄H₂₈ClFN₂O₄ + H]⁺. |
| 89 | B | 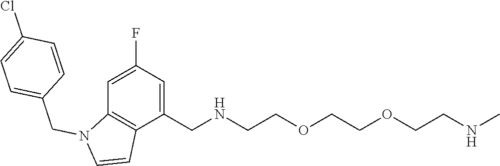 [SMILES: CNCCOCCOCCNCc1cc(F)cc2c1ccn2Cc1ccc(Cl)cc1] | ¹H NMR (400 MHz, DMSO-d₆) δ (ppm): 9.43 (br. s, 2H; HCl), 8.90 (br. s, 2H), 7.63 (d, J = 3.2 Hz, 1H), 7.47 (dd, J = 10.0 Hz, 1.6 Hz, 1H), 7.39 (d, J = 8.4 Hz, 2H), 7.29 (dd, J = 10.4 Hz, 2.0 Hz, 1H), 7.24 (d, J = 8.4 Hz, 2H), 6.81 (d, J = 2.8 |
| | | | Hz, 1H), 5.43 (s, 2H), |
| | | | 4.43 (t, J = 5.2 Hz, 2H), |
| | | | 3.75 (t, J = 5.2 Hz, 2H), |
| | | | 3.69 (t, J = 5.2 Hz, 2H), |
| | | | 3.60 (s, 4H), 3.14-3.03 |
| | | | (m, 4H), 2.54 (s, 3H). |
| | | | MS (ESI) m/z 434.2 |
| | | | [C₂₃H₂₉ClFN₃O₂ + H]⁺. |
| 90 | B | 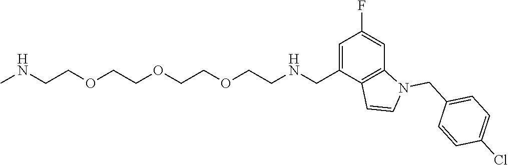 [SMILES: CNCCOCCOCCOCCNCc1cc(F)cc2c1ccn2Cc1ccc(Cl)cc1] | ¹H NMR (400 MHz, DMSO-d₆) δ (ppm): 7.57 (d, J = 3.2 Hz, 1H), 7.40- 7.34 (m, 3H), 7.23 (d, J = 8.4 Hz, 2H), 7.09 (dd, J = 10.4 Hz, 2.0 Hz, 1H), 6.71 (d, J = 3.2 Hz, 1H), 5.41 (s, 2H), 4.22 (s, 2H), 3.65-3.61 (m, 4H), 3.55- 3.24 (br. s, 8H), 3.02 (t, |
| | | | J = 5.6 Hz, 2H), 2.94 (t, |
| | | | J = 5.6 Hz, 2H), 2.52 (s, |
| | | | 3H). MS (ESI) m/z 478.2 |
| | | | [C₂₅H₃₃ClFN₃O₃ + H]⁺. |
| 91 | B | 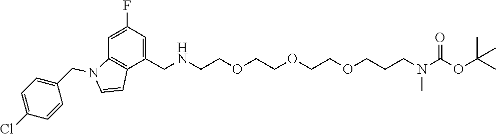 [SMILES: CN(CCCOCCOCCOCCNCc1cc(F)cc2c1ccn2Cc1ccc(Cl)cc1)C(=O)OC(C)(C)C] | ¹H NMR (400 MHz, DMSO-d₆) δ (ppm): 7.47 (d, J = 3.2 Hz, 1H), 7.37 (d, J = 8.4 Hz, 2H), 7.22- 7.17 (m, 3H), 6.89 (dd, J = 10.8 Hz, 2.0 Hz, 1H), 6.58 (d, J = 3.2 Hz, 1H), 5.38 (s, 2H), 3.95 (s, 2H), |
| | | | 3.51-3.43 (m, 10H), 3.35- |
| | | | 3.33 (m, 2H) 3.16 (t, J = |
| | | | 7.2 Hz, 2H), 2.73 (s, 3H), |
| | | | 2.68 (t, J = 5.6 Hz, 2H), |
| | | | 2.08 (br, 1H), 1.66-1.63 |
| | | | (m, 2H), 1.36 (s, 9H). |
| | | | MS (ESI) m/z 592.3 |
| | | | [C₃₁H₄₃ClFN₃O₅ + H]⁺. |
| 92 | B | 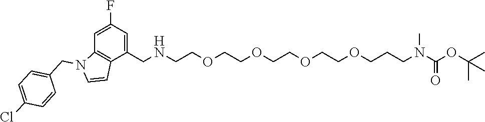 [SMILES: CN(CCCOCCOCCOCCOCCNCc1cc(F)cc2c1ccn2Cc1ccc(Cl)cc1)C(=O)OC(C)(C)C] | ¹H NMR (400 MHz, DMSO-d₆) δ (ppm): 7.47 (d, J = 3.2 Hz, 1H), 7.38- 7.36 (m, 2H), 7.22-7.18 (m, 3H), 6.89 (dd, J = 10.8 Hz, 2.0 Hz, 1H), 6.59 (d, J = 2.8 Hz, 1H), 5.38 (s, 2H), 3.95 (s, 2H), |
| | | | 3.49-3.42 (m, 14H), 3.34 |
| | | | (m, 2H), 3.17 (t, J = 7.2 |
| | | | Hz, 2H), 2.74 (s, 3H), |
| | | | 2.69 (t, J = 6.0 Hz, 2H), |
| | | | 1.66-1.63 (t, J = 6.4 Hz, |
| | | | 2H), 1.37 (s, 9H). MS |
| | | | (ESI) m/z 636.2 |
| | | | [C₃₃H₄₇ClFN₃O₆ + H]⁺. |
| 93 | B | 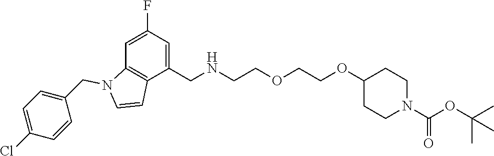 [SMILES: CC(C)(C)OC(=O)N1CCC(OCCOCCNCc2cc(F)cc3c2ccn3Cc2ccc(Cl)cc2)CC1] | ¹H NMR (400 MHz, DMSO-d₆) δ (ppm): 7.46 (d, J = 3.2 Hz, 1H), 7.37 (d, J = 8.4 Hz, 2H), 7.22- 7.17 (m, 3H), 6.89 (dd, J = 10.8 Hz, 2.4 Hz, 1H), 6.58 (d, J = 3.2 Hz, 1H), 5.37 (s, 2H), 3.95 (s, 2H), 3.62-3.56 (m, 2H), 3.51- 3.48 (m, 6H), 3.45-3.41 |
| | | | (m, 1H), 3.01-2.90 (m, |
| | | | 2H), 2.68 (t, J = 5.6 Hz, |
| | | | 2H), 2.08 (br, 1H), 1.77- |
| | | | 1.71 (m, 2H), 1.37 (s, |
| | | | 9H), 1.32-1.24 (m, 2H). |
| | | | MS (ESI) m/z 560.1 |
| | | | [C₃₀H₃₉ClFN₃O₄ + H]⁺. |
| 94 | B | 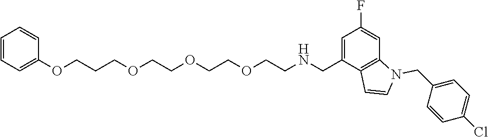 [SMILES: Fc1cc(CNCCOCCOCCOCCCOc2ccccc2)c2ccn(Cc3ccc(Cl)cc3)c2c1] | ¹H NMR (400 MHz, DMSO-d₆) δ (ppm): 7.47 (d, J = 3.2 Hz, 1H), 7.38- 7.36 (m, 2H), 7.27-7.17 (m, 5H), 6.91-6.88 (m, 4H), 6.58 (d, J = 3.2 Hz, 1H), 5.37 (s, 2H), 3.97 (t, J = 6.0 Hz, 2H), 3.94 (s, 2H), 3.53-3.46 (m, 12H), |
| | | | 2.69-2.66 (m, 2H), 2.10 |
| | | | (br, 1H), 1.92-1.89 (m, |
| | | | 2H). MS (ESI) m/z 555.2 |
| | | | [C₃₁H₃₆ClFN₂O₄ + H]⁺. |
| 95 | B | 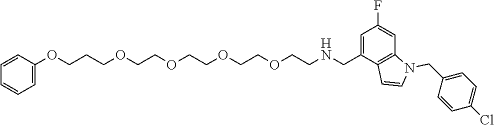 [SMILES: Fc1cc(CNCCOCCOCCOCCOCCCOc2ccccc2)c2ccn(Cc3ccc(Cl)cc3)c2c1] | ¹H NMR (400 MHz, DMSO-d₆) δ (ppm): 7.46 (d, J = 3.2 Hz, 1H), 7.38- 7.36 (m, 2H), 7.28-7.24 (m, 2H), 7.22-7.17 (m, 3H), 6.92-6.88 (m, 4H), 6.58 (d, J = 2.4 Hz, 1H), 5.37 (s, 2H), 3.98 (t, J = |
| | | | 6.4 Hz, 2H), 3.94 (s, 2H), |
| | | | 3.52 (t, J = 6.4 Hz, 2H), |
| | | | 3.49-3.47 (m, 14H), |
| | | | 2.69-2.67 (m, 2H), 2.11 |
| | | | (br, 1H), 1.94-1.88 (m, |
| | | | 2H). MS (ESI) m/z 599.1 |
| | | | [C₃₃H₄₀ClFN₂O₅ + H]⁺. |
| 96 | B | 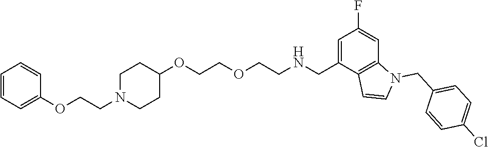 [SMILES: Fc1cc(CNCCOCCOC2CCN(CCOc3ccccc3)CC2)c2ccn(Cc3ccc(Cl)cc3)c2c1] | ¹H NMR (400 MHz, DMSO-d₆) δ (ppm): 7.46 (d, J = 3.2 Hz, 1H), 7.37 (d, J = 6.4 Hz, 2H), 7.29- 7.17 (m, 5H), 6.93-6.88 (m, 4H), 6.59 (d, J = 2.8 Hz, 1H), 5.37 (s, 2H), 4.02 (t, J = 6.0 Hz, 2H), 3.95 (s, 2H), 3.51-3.48 |
| | | | (m, 6H), 3.28-3.22 (m, |
| | | | 1H), 2.75-2.67 (m, 4H), |
| | | | 2.62 (t, J = 5.6 Hz, 2H), |
| | | | 2.13-2.08 (m, 2H), 1.81- |
| | | | 1.73 (m, 2H), 1.41-1.33 |
| | | | (m, 2H). MS (ESI) m/z |
| | | | 580.2[C₃₃H₃₉ClFN₃O₃ + |
| | | | H]⁺. |
| 117 | E | 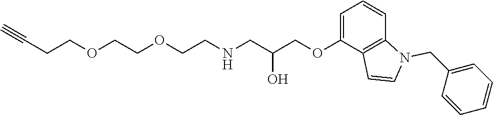 [SMILES: C#CCCOCCOCCNCC(O)COc1cccc2c1ccn2Cc1ccccc1] | ¹H NMR (400 MHz, DMSO-d₆) δ (ppm): 8.25 (br. s, 1H; HCOOH), 7.38 (d, J = 3.2 Hz, 1H), 7.31-7.27 (m, 2H), 7.24- 7.21 (m, 1H), 7.16-7.14 (m, 2H), 7.03-6.96 (m, 2H), 6.51-6.49 (m, 2H), |
| | | | 5.38 (s, 2H), 4.01 (s, 3H), |
| | | | 3.57-3.51 (m, 6H), 3.48 |
| | | | (t, J = 6.8 Hz, 2H), 2.87- |
| | | | 2.70 (m, 5H), 2.39-2.33 |
| | | | (m, 2H). MS (ESI) m/z |
| | | | 437.1[C₂₆H₃₂N₂O₄ + H]⁺. |
| 118 | E | 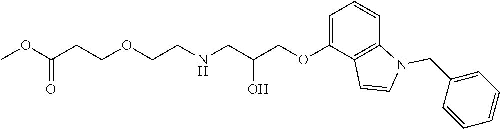 [SMILES: COC(=O)CCOCCNCC(O)COc1cccc2c1ccn2Cc1ccccc1] | ¹H NMR (400 MHz, DMSO-d₆) δ (ppm): 7.36 (d, J = 3.2 Hz, 1H), 7.31- 7.27 (m, 2H), 7.24-7.21 (m, 1H), 7.16-7.14 (m, 2H), 7.02-6.96 (m, 2H), 6.50-6.49 (m, 2H), 5.38 (s, 2H), 4.98 (br. s, 1H), |
| | | | 4.02-3.94 (m, 3H), 3.62- |
| | | | 3.56 (m, 5H), 3.44 (t, J = |
| | | | 6.0 Hz, 2H), 2.75-2.60 |
| | | | (m, 4H), 2.55-2.51 (m, |
| | | | 2H), 1.88 (br, 1H). MS |
| | | | (ESI) m/z 427.2 |
| | | | [C₂₄H₃₀N₂O₅ + H]⁺. |
| 119 | B | 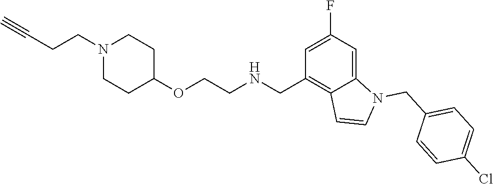 [SMILES: C#CCCN1CCC(OCCNCc2cc(F)cc3c2ccn3Cc2ccc(Cl)cc2)CC1] | ¹H NMR (400 MHz, DMSO-d₆) δ (ppm): 7.49 (d, J = 3.2 Hz, 1H), 7.37 (d, J = 8.4 Hz, 2H), 7.22 (d, J = 8.4 Hz, 3H), 6.92 (d, J = 9.2 Hz, 1H), 6.61 (d, J = 3.2 Hz, 1H), 5.38 (s, 2H), 4.00 (s, 2H), 3.48 (t, J = 5.6 Hz, 2H), 3.26- 3.21 (m, 1H), 2.76 (t, J = 2.4 Hz, 1H), 2.73-2.65 (m, 4H), 2.43 (t, J = 7.6 |
| | | | Hz, 2H), 2.23-2.26 (m, |
| | | | 2H), 2.09-2.04 (m, 2H), |
| | | | 1.78-1.76 (m, 2H). 1.39- |
| | | | 1.36 (m, 2H). MS |
| | | | (ESI) m/z 468.1 |
| | | | [C₂₇H₃₁ClFN₃O + H]⁺. |
| 120 | B | 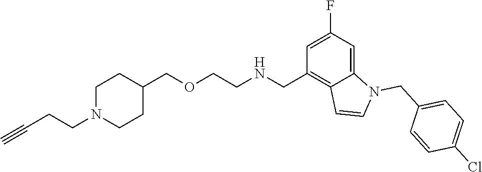 [SMILES: C#CCCN1CCC(COCCNCc2cc(F)cc3c2ccn3Cc2ccc(Cl)cc2)CC1] | ¹H NMR (400 MHz, DMSO-d₆) δ (ppm): 7.46 (d, J = 3.2 Hz, 1H), 7.37 (dt, J = 9.2 Hz, 2.4 Hz, 2H), 7.22-7.17 (m, 3H), 6.89 (dd, J = 10.8 Hz, 2.0 Hz, 1H), 6.58 (dd, J = 3.2 Hz, 0.8 Hz, 1H), 5.38 (s, 2H), 3.95 (s, 2H), 3.43 (t, J = 5.6 Hz, 2H), 3.19 (d, J = 6.4 Hz, 2H), 2.81 (d, |
| | | | J = 11.2 Hz, 2H), 2.75 (t, |
| | | | J = 2.4 Hz, 1H), 2.66 (t, |
| | | | J = 2.0 Hz, 2H), 2.44-2.40 |
| | | | (m, 2H), 2.28 (dt, J = 7.6 |
| | | | Hz, 2.4 Hz, 2H), 2.08 (br. |
| | | | s, 1H), 1.88 (dt, J = 11.6 |
| | | | Hz, 2.4 Hz, 2H), 1.58 (d, |
| | | | J = 13.2 Hz, 2H), 1.49- |
| | | | 1.41 (m, 1H), 1.12-1.08 |
| | | | (m, 2H). MS (ESI) m/z |
| | | | 482.3[C₂₈H₃₃ClFN₃O + |
| | | | H]⁺. |
| 121 | B | 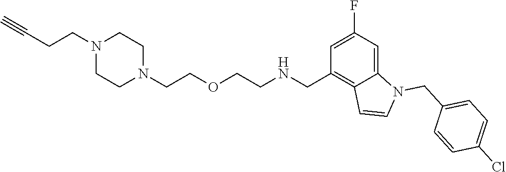 [SMILES: C#CCCN1CCN(CCOCCNCc2cc(F)cc3c2ccn3Cc2ccc(Cl)cc2)CC1] | ¹H NMR (400 MHz, DMSO-d₆) δ (ppm): 8.19 (s, 1H, HCOOH), 7.48 (d, J = 3.2 Hz, 1H), 7.37 (d, J = 8.4 Hz, 2H), 7.23- 7.21 (m, 3H), 6.91 (dd, J = 10.8 Hz, 2.0 Hz, 1H), 6.60 (d, J = 3.2 Hz, 1H), 5.38 (s, 2H), 3.99 (s, 2H), 3.47 (q, J = 6.0 Hz, 4H), 2.76 (t, J = 2.4 Hz, |
| | | | 1H), 2.72 (t, J = 5.6 Hz, |
| | | | 2H), 2.58-2.51 (m, 4H), |
| | | | 2.43 (t, J = 5.6 Hz, 2H), |
| | | | 2.40-2.26 (m, 8H). MS |
| | | | (ESI) m/z 497.2 |
| | | | [C₂₈H₃₄ClFN₄O + H]⁺. |
| 122 | B | 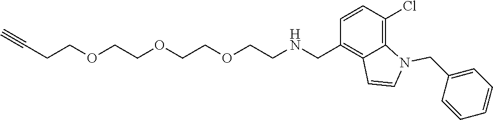 [SMILES: C#CCCOCCOCCOCCNCc1ccc(Cl)c2c1ccn2Cc1ccccc1] | ¹H NMR (400 MHz, DMSO-d₆) δ (ppm): 7.54 (d, J = 3.2 Hz, 1H), 7.30- 7.26 (m, 2H), 7.23-7.21 (m, 1H), 7.06-7.01 (m, 2H), 6.99-6.97 (m, 2H), 6.71 (d, J = 3.2 Hz, 1H) 5.77 (s, 2H), 3.95 (s, 2H), |
| | | | 3.52-3.45 (m, 12H), 2.77 |
| | | | (t, J = 3.2 Hz, 1H), 2.70 |
| | | | (t, J = 5.6 Hz, 2H), 2.38- |
| | | | 2.34 (m, 2H), 2.14 (br, |
| | | | 1H). MS (ESI) m/z 455.2 |
| | | | [C₂₆H₃₁ClN₂O₃ + H]⁺. |
| 123 | B | 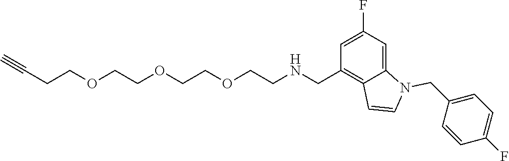 [SMILES: C#CCCOCCOCCOCCNCc1cc(F)cc2c1ccn2Cc1ccc(F)cc1] | ¹H NMR (400 MHz, DMSO-d₆) δ (ppm): 7.47 (d, J = 3.2 Hz, 1H), 7.29- 7.24 (m, 2H), 7.23-7.20 (m, 1H), 7.17-7.12 (m, 2H), 6.91-6.87 (m, 1H), 6.58-6.57 (m, 1H), 5.36 (s, 2H), 3.95 (s, 2H), 3.52-3.44 (m, 12H), 2.78 (t, J = 2.8 Hz, 1H), 2.68 |
| | | | (t, J = 5.6 Hz, 2H), 2.38- |
| | | | 2.33 (m, 2H), 2.12 (br. s, |
| | | | 1H). MS (ESI) m/z 457.3 |
| | | | [C₂₆H₃₀F₂N₂O₃ + H]⁺. |
| 124 | B | 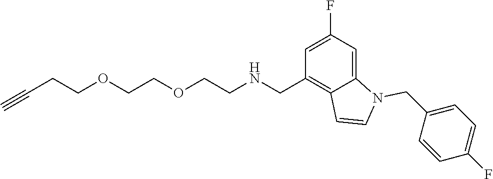 [SMILES: C#CCCOCCOCCNCc1cc(F)cc2c1ccn2Cc1ccc(F)cc1] | ¹H NMR (400 MHz, DMSO-d₆) δ (ppm): 7.48 (d, J = 3.2 Hz, 1H), 7.29- 7.22 (m, 3H), 7.16-7.12 (m, 2H), 6.91 (dd, J = 10.8 Hz, 2.0 Hz, 1H), 6.59 (dd, J = 3.2 Hz, 0.8 Hz, 1H), 5.36 (s, 2H), 3.98 (s, 2H), 3.52-3.46 (m, 8H), 2.77 (t, J = 2.4 Hz, 1H), 2.71 (t, J = 5.6 |
| | | | Hz, 2H), 2.38-2.34 (m, |
| | | | 2H). MS (ESI) m/z 413.2 |
| | | | [C₂₄H₂₆F₂N₂O₂ + H]⁺. |
| 125 | B | 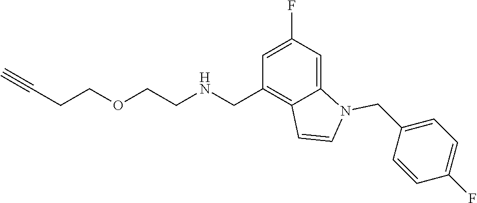 [SMILES: C#CCCOCCNCc1cc(F)cc2c1ccn2Cc1ccc(F)cc1] | ¹H NMR (400 MHz, DMSO-d₆) δ (ppm): 7.48 (d, J = 3.2 Hz, 1H), 7.28 (dd, J = 8.4 Hz, 5.6 Hz, 2H), 7.22 (d, J = 8.0 Hz, 1H), 7.14 (t, J = 8.8 Hz, 2H), 6.90 (d, J = 10.8 Hz, 1H), 6.59 (d, J = 2.8 Hz, 1H), 5.37 (s, 2H), 3.95 (s, 2H), 3.51-3.45 (m, 4H), 2.78 (d, J = 2.4 Hz, 1H), 2.70-2.67 (m, 2H), 2.38 (dt, J = 6.8 Hz, 2.4 |
| | | | Hz, 2H). MS (ESI) m/z |
| | | | 369.1[C₂₂H₂₂F₂N₂O + H]⁺. |
| 126 | A | 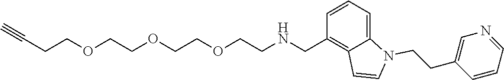 [SMILES: C#CCCOCCOCCOCCNCc1cccc2c1ccn2CCc1cccnc1] | ¹H NMR (400 MHz, DMSO-d₆) δ (ppm): 8.39- 8.35 (m, 2H), 7.60-5.57 (m, 1H), 7.37 (d, J = 8.0 Hz, 1H), 7.28-7.24 (m, |
| | | | 2H), 7.06 (t, J = 7.2 Hz, |
| | | | 1H), 6.98 (d, J = 7.2 Hz, |
| | | | 1H), 6.49 (d, J = 2.8 Hz, |
| | | | 1H), 4.42 (t, J = 7.2 Hz, |
| | | | 2H), 3.94 (s, 2H), 3.51- |
| | | | 3.44 (m, 12H), 3.08 (t, |
| | | | J = 7.2 Hz, 2H), 2.78 (t, |
| | | | J = 2.4 Hz, 1H), 2.69 (t, |
| | | | J = 6.0 Hz, 2H), 2.38- |
| | | | 2.34 (m, 2H). MS (ESI) |
| | | | m/z 436.2[C₂₆H₃₃N₃O₃ + |
| | | | H]⁺. |
| 127 | B | 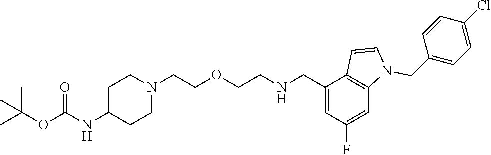 [SMILES: CC(C)(C)OC(=O)NC1CCN(CCOCCNCc2cc(F)cc3c2ccn3Cc2ccc(Cl)cc2)CC1] | ¹H NMR (400 MHz, DMSO-d₆) δ (ppm): 7.47 (d, J = 3.2 Hz, 1H), 7.39- 7.36 (m, 2H), 7.22-7.17 (m, 3H), 6.89 (dd, J = 10.8 Hz, 2.4 Hz, 1H), 6.71 (d, J = 7.6 Hz, 1H), 6.59 (dd, J = 3.2 Hz, 0.8 Hz, 1H), 5.38 (s, 2H), 3.95 (s, 2H) 3.47-3.42 |
| | | | (m, 4H), 3.17-3.14 (m, |
| | | | 1H), 2.79-2.76 (m, 2H), |
| | | | 2.67 (t, J = 5.6 Hz, 2H), |
| | | | 2.40 (t, J = 6.0 Hz, 2H), |
| | | | 2.16 (br. s, 1H), 1.93 (t, |
| | | | J = 11.2 Hz, 2H), 1.63 (d, |
| | | | J = 11.2 Hz, 2H), 1.37 (s, |
| | | | 9H), 1.36-1.30 (m, 2H) |
| | | | MS (ESI) m/z 559.4 |
| | | | [C₃₀H₄₀ClFN₄O₃ + H]⁺. |
| 128 | B | 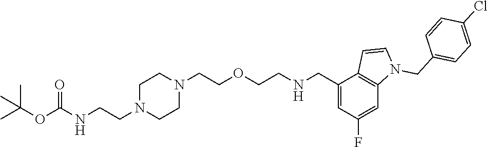 [SMILES: CC(C)(C)OC(=O)NCCN1CCN(CCOCCNCc2cc(F)cc3c2ccn3Cc2ccc(Cl)cc2)CC1] | ¹H NMR (400 MHz, DMSO-d₆) δ ppm: 7.47 (d, J = 3.2 Hz, 1H), 7.37 (d, J = 6.4 Hz, 2H), 7.21 (d, J = 8.4 Hz, 2H), 7.19- 7.17 (m, 1H), 6.89 (dd, J = 10.8 Hz, 2.0 Hz, 1H), 6.61-6.58 (m, 2H), 5.38 (s, 2H), 3.95 (s, 2H), |
| | | | 3.47-3.44 (m, 4H), 2.98 |
| | | | (q, J = 6.4 Hz, 2H), 2.67 |
| | | | (t, J = 5.6 Hz, 2H), 2.41 |
| | | | (t, J = 6.0 Hz, 2H), 2.38- |
| | | | 2.27 (m, 8H), 2.25 (t, J = |
| | | | 6.8 Hz, 2H), 1.36 (s, 9H). |
| | | | MS (ESI) m/z 588.3 |
| | | | [C₃₁H₄₃ClFN₅O₃ + H]⁺. |
| 129 | B | 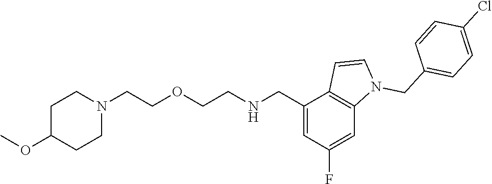 [SMILES: COC1CCN(CCOCCNCc2cc(F)cc3c2ccn3Cc2ccc(Cl)cc2)CC1] | ¹H NMR (400 MHz, DMSO-d₆) δ (ppm): 7.47 (d, J = 3.2 Hz, 1H), 7.37 (d, J = 8.4 Hz, 2H), 7.22- 7.17 (m, 3H), 6.89 (dd, J = 10.8 Hz, 2.0 Hz, 1H), 6.59 (d, J = 2.4, 1H), 5.38 (s, 2H), 3.95 (s, 2H), 3.48-3.44 (m, 4H), 3.19 (s, 3H), 3.10-3.06 (m, 1H), 2.69-2.65 (m, 4H), 2.42 (t, J = 6.0 Hz, 2H), |
| | | | 2.09-2.03 (m, 2H), 1.78- |
| | | | 1.73 (m, 2H), 1.38-1.30 |
| | | | (m, 2H). MS (ESI) m/z |
| | | | 474.3[C₂₆H₃₃ClFN₃O₂ + |
| | | | H]⁺. |
| 130 | B | 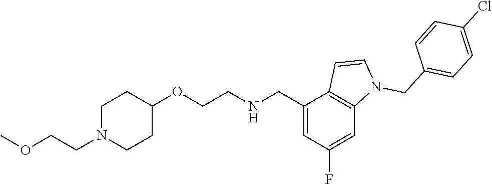 [SMILES: COCCN1CCC(OCCNCc2cc(F)cc3c2ccn3Cc2ccc(Cl)cc2)CC1] | ¹H NMR (400 MHz, DMSO-d₆) δ (ppm): 7.47 (d, J = 3.2 Hz, 1H), 7.39- 7.36 (m, 2H), 7.23-7.17 (m, 3H), 6.90 (dd, J = 10.8 Hz, 2.0 Hz, 1H), 6.59 (d, J = 3.2 Hz, 1H), 5.38 (s, 2H), 3.95 (s, 2H), 3.46 (t, J = 5.6 Hz, 2H), 3.38 (t, J = 6.0 Hz, 2H), 3.27-3.19 (m, 4H), 2.69- 2.63 (m, 4H), 2.41 (t, J = |
| | | | 5.6 Hz, 2H), 2.04 (t, J = |
| | | | 9.6 Hz, 2H), 1.79-1.74 |
| | | | (m, 2H), 1.39-1.33 (m, |
| | | | 2H). MS (ESI) m/z 474.3 |
| | | | [C₂₆H₃₃ClFN₃O₂ + H]⁺. |
| 131 | B | 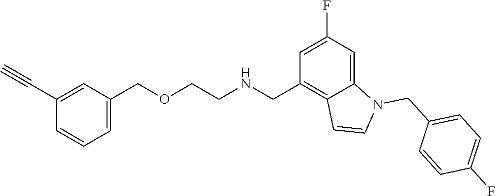 [SMILES: C#Cc1cccc(COCCNCc2cc(F)cc3c2ccn3Cc2ccc(F)cc2)c1] | ¹H NMR (400 MHz, DMSO-d₆) δ ppm: 7.47 (d, J = 3.2 Hz, 1H), 7.43 (s, 1H), 7.40-7.35 (m, 3H), 7.29-7.21 (m, 3H), 7.16-7.11 (m, 2H), 6.91 (dd, J = 10.8, 2.4 Hz, 1H), 6.58 (d, J = 2.8 Hz, 1H), 5.36 (s, 2H), 4.47 (s, 2H), 4.18 (s, 1H), 3.98 (s, 2H), 3.54 (t, J = 5.6 Hz, 2H), 2.76 (t, J = 5.6 Hz, 2H). |
| | | | MS (ESI) m/z 431.2 |
| | | | [C₂₇H₂₄F₂N₂O + H]⁺. |
| 132 | B | 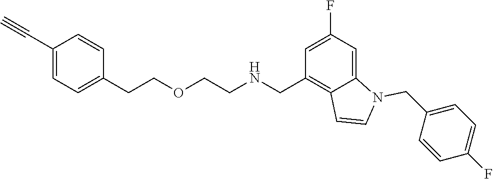 [SMILES: C#Cc1ccc(CCOCCNCc2cc(F)cc3c2ccn3Cc2ccc(F)cc2)cc1] | ¹H NMR (400 MHz, DMSO-d₆) δ ppm: 7.47 (d, J = 3.2 Hz, 1H), 7.36 (d, J = 8.0 Hz, 2H), 7.29- 7.21 (m, 5H), 7.13 (dt, J = 8.8, 2.0 Hz, 2H), 6.87 (dd, J = 10.8, 2.0 Hz, 1H), 6.56 (d, J = 2.8 Hz, 1H), 5.36 (s, 2H), 4.10 (s, 1H), 3.93 (s, 2H), 3.57 (t, J = 6.8 Hz, 2H), 3.47 (t, J = |
| | | | 5.6 Hz, 2H), 2.80 (t, J = |
| | | | 6.8 Hz, 2H), 2.68 (t, J = |
| | | | 5.6 Hz, 2H). MS (ESI) |
| | | | m/z 445.2[C₂₈H₂₆F₂N₂O + |
| | | | H]⁺. |
| 133 | B | 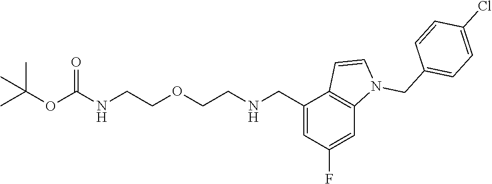 [SMILES: CC(C)(C)OC(=O)NCCOCCNCc1cc(F)cc2c1ccn2Cc1ccc(Cl)cc1] | ¹H NMR (400 MHz, CDCl₃) δ (ppm): 7.27 (d, J = 6.4 Hz, 2 H), 7.10 (d, J = 3.2 Hz, 1H), 7.06- 6.99 (m, 2H), 6.92 (dd, J = 10.4 Hz, 2.4 Hz, 1H), 6.80 (dd, J = 9.6 Hz, 2.4 Hz, 1H), 6.60 (d, J = 3.2 Hz, 1H), 5.22 (s, 2H), 5.00 (s, 1H), 4.09 (s, 2H), 3.59 (t, J = 5.2 Hz, 2H) 3.50 (t, J = 5.2 Hz, 2H), |
| | | | 3.30 (q, J = 5.6 Hz, 2H), |
| | | | 2.86 (t, J = 5.2 Hz, 2H), |
| | | | 1.43 (s, 9H). MS |
| | | | (ESI) m/z 476.5 |
| | | | [C₂₅H₃₁ClFN₃O₃ + H]⁺. |
| 134 | B | 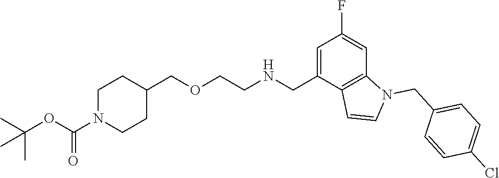 [SMILES: CC(C)(C)OC(=O)N1CCC(COCCNCc2cc(F)cc3c2ccn3Cc2ccc(Cl)cc2)CC1] | ¹H NMR (400 MHz, CDCl₃) δ (ppm): 7.30- 7.22 (m, 2H), 7.10 (s, 1H), 7.05-6.98 (m, 2H), 6.91 (dd, J = 10.4 Hz, 2.4 Hz, 1H), 6.81 (dd, J = 9.6 Hz, 2.4 Hz, 1H), 6.59 (dd, J = 3.2 Hz, 0.8 Hz, 1H), 5.21 (s, 2H), 4.11 (s, 2H), 4.07 (s, 2H), 3.56 (t, J = 5.2 Hz, 2H), 3.25 (d, J = |
| | | | 6.0 Hz, 2H), 2.87 (t, J = |
| | | | 5.2 Hz, 2H), 2.67 (t, J = |
| | | | 12.8 Hz, 2H), 2.01 (s, |
| | | | 1H), 1.77-1.62 (m, 2H), |
| | | | 1.45 (s, 9H), 1.18-1.03 |
| | | | (m, 2H). MS (ESI) m/z |
| | | | 530.7[C₂₉H₃₇ClFN₃O₃ + |
| | | | H]⁺. |
| 135 | B | 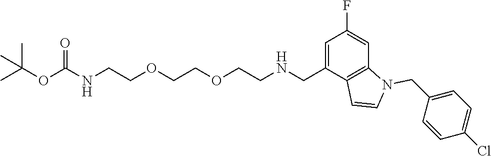 [SMILES: CC(C)(C)OC(=O)NCCOCCOCCNCc1cc(F)cc2c1ccn2Cc1ccc(Cl)cc1] | ¹H NMR (400 MHz, CDCl₃) δ (ppm): 7.31- 7.23 (m, 2H), 7.09 (d, J = 3.2 Hz, 1H), 7.06-6.98 (m, 2H), 6.92 (dd, J = 10.4 Hz, 2.4 Hz, 1H), 6.83-6.76 (m, 1H), 6.60 (dd, J = 3.2 Hz, 0.8 Hz, 1H), 5.21 (s, 2H), 5.13 (s, 1H), 4.08 (s, 2H), 3.67- |
| | | | 3.60 (m, 2H), 3.60 (s, |
| | | | 4H), 3.52 (t, J = 5.2 Hz, |
| | | | 2H), 3.29 (q, J = 5.6 Hz, |
| | | | 2H), 2.88 (t, J = 5.2 Hz, |
| | | | 2H), 1.43 (s, 9H). MS |
| | | | (ESI) m/z 520.5 |
| | | | [C₂₇H₃₅ClFN₃O₄ + H]⁺. |
| 136 | B | 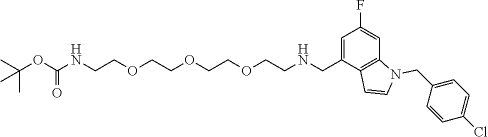 [SMILES: CC(C)(C)OC(=O)NCCOCCOCCOCCNCc1cc(F)cc2c1ccn2Cc1ccc(Cl)cc1] | ¹H NMR (400 MHz, CDCl₃) δ (ppm): 7.30- 7.24 (m, 2H), 7.08 (d, J = 3.2 Hz, 1H), 7.05-6.99 (m, 2H), 6.92 (dd, J = 10.4 Hz, 2.4 Hz, 1H), 6.79 (dd, J = 9.6 Hz, 2.4 Hz, 1H), 6.60 (d, J = 3.2 Hz, 1H), 5.28 (s, 1H), |
| | | | 5.22 (s, 2H), 4.08 (s, 2H), |
| | | | 3.68-3.55 (m, 10H), 3.50 |
| | | | (t, J = 5.2 Hz, 2H), 3.28 |
| | | | (q, J = 5.6 Hz, 2H), 2.88 |
| | | | (t, J = 5.2 Hz, 2H), 1.43 |
| | | | (s, 9H). MS (ESI) m/z |
| | | | 564.7[C₂₉H₃₉ClFN₃O₅ + |
| | | | H]⁺. |
| 137 | B | 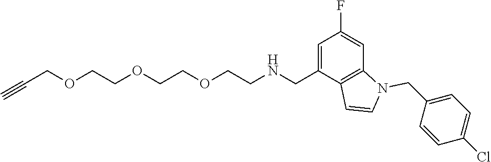 [SMILES: C#CCOCCOCCOCCNCc1cc(F)cc2c1ccn2Cc1ccc(Cl)cc1] | ¹H NMR (400 MHz, CDCl₃) δ (ppm): 7.31- 7.23 (m, 2H), 7.08 (d, J = 3.2 Hz, 1H), 7.06-6.99 (m, 2H), 6.92 (dd, J = 10.4 Hz, 2.4 Hz, 1H), 6.79 (dd, J = 9.6 Hz, 2.4 Hz, 1H), 6.60 (dd, J = 3.2 Hz, 0.8 Hz, 1H), 5.21 (s, 2H), 4.18 (d, J = 2.4 |
| | | | Hz, 2H), 4.08 (s, 2H), |
| | | | 3.72-3.57 (m, 10H), 2.88 |
| | | | (t, J = 5.2 Hz, 2H), 2.41 |
| | | | (t, J = 2.4 Hz, 1H). MS |
| | | | (ESI) m/z 459.5 |
| | | | [C₂₅H₂₈ClFN₂O₃ + H]⁺. |
| 138 | B | 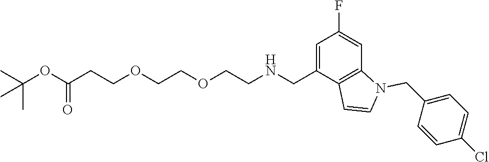 [SMILES: CC(C)(C)OC(=O)CCOCCOCCNCc1cc(F)cc2c1ccn2Cc1ccc(Cl)cc1] | ¹H NMR (400 MHz, CDCl₃) δ (ppm): 7.31- 7.23 (m, 2H), 7.08 (d, J = 3.2 Hz, 1H), 7.06-6.98 (m, 2H), 6.92 (dd, J = 10.4 Hz, 2.4 Hz, 1H), 6.79 (dd, J = 9.6 Hz, 2.4 Hz, 1H), 6.60 (dd, J = 3.2 Hz, 0.8 Hz, 1H), 5.21 (s, 2H), 4.08 (s, |
| | | | 2H), 3.70 (t, J = 6.4 Hz, |
| | | | 2H), 3.66-3.56 (m, 2H), |
| | | | 3.60 (s, 4H), 2.87 (t, J = |
| | | | 5.2 Hz, 2H), 2.49 (t, J = |
| | | | 6.4 Hz, 2H), 1.44 (s, |
| | | | 9H). MS (ESI) m/z 505.6 |
| | | | [C₂₇H₃₄ClFN₂O₄ + H]⁺. |
| 139 | B | 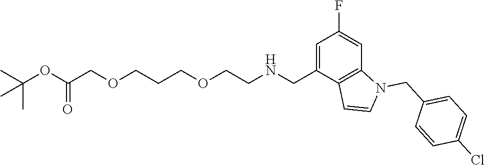 [SMILES: CC(C)(C)OC(=O)COCCCOCCNCc1cc(F)cc2c1ccn2Cc1ccc(Cl)cc1] | ¹H NMR (400 MHz, CDCl₃) δ (ppm): 7.31- 7.23 (m, 2H), 7.09 (d, J = 3.2 Hz, 1H), 7.05-6.99 (m, 2H), 6.92 (dd, J = 10.4 Hz, 2.4 Hz, 1H), 6.79 (dd, J = 9.6 Hz, 2.4 Hz, 1H), 6.60 (dd, J = 3.2 Hz, 0.8 Hz, 1H), 5.22 (s, 2H), 4.09 (s, 2H), 3.93 |
| | | | (s, 2H), 3.63-3.51 (m, |
| | | | 6H), 2.86 (t, J = 5.2 Hz, |
| | | | 2H), 1.89 (p, J = 6.4 Hz, |
| | | | 2H), 1.47 (s, 9H). MS |
| | | | (ESI) m/z 505.6 |
| | | | [C₂₇H₃₄ClFN₂O₄ +H]⁺. |
| 140 | B | 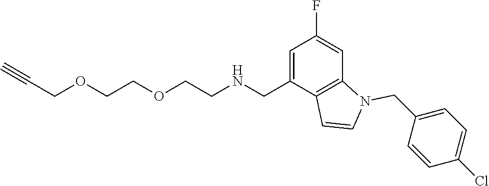 [SMILES: C#CCOCCOCCNCc1cc(F)cc2c1ccn2Cc1ccc(Cl)cc1] | ¹H NMR (400 MHz, CDCl₃) δ (ppm): 7.33- 7.23 (m, 2H), 7.08 (d, J = 3.2 Hz, 1H), 7.06-6.98 (m, 2H), 6.93 (dd, J = 10.4 Hz, 2.4 Hz, 1H), 6.79 (dd, J = 9.6 Hz, 2.4 Hz, 1H), 6.60 (dd, J = 3.2 Hz, 0.8 Hz, 1H), 5.22 (s, 2H), 4.19 (d, J = 2.4 Hz, 2H), 4.08 (s, 2H), 3.74- 3.67 (m, 2H), 3.67-3.60 |
| | | | (m, 4H), 2.89 (t, J = 5.2 |
| | | | Hz, 2H), 2.41 (t, J = 2.4 |
| | | | Hz, 1H). MS (ESI) m/z |
| | | | 415.4[C₂₃H₂₄ClFN₂O₂ + |
| | | | H]⁺. |
| 141 | B | 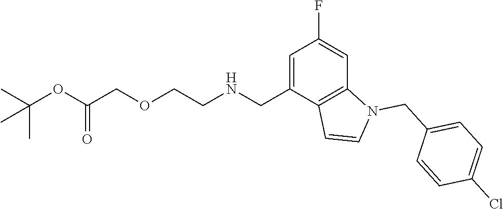 [SMILES: CC(C)(C)OC(=O)COCCNCc1cc(F)cc2c1ccn2Cc1ccc(Cl)cc1] | ¹H NMR (400 MHz, CDCl₃) δ (ppm): 7.27 (d, J = 6.4 Hz, 2H), 7.09 (d, J = 3.2 Hz, 1H), 7.05- 6.99 (m, 2H), 6.95 (dd, J = 10.4 Hz, 2.4 Hz, 1H), 6.81 (dd, J = 9.2 Hz, 2.4 Hz, 1H), 6.62 (dd, J = 3.2 Hz, 0.8 Hz, 1H), 5.22 (s, 2H), 4.13 (s, 2H), 3.98 (s, 2H), 3.71 (t, J = 5.2 Hz, 2H), 2.94 (t, J = 5.2 Hz, 2H), 1.47 (s, 9H). |
| | | | MS (ESI) m/z 447.6 |
| | | | [C₂₄H₂₈ClFN₂O₃ + H]⁺. |
| 142 | B | 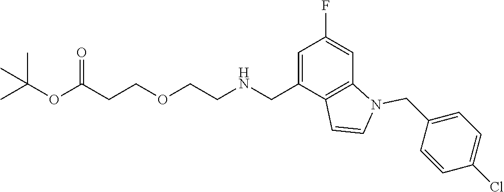 [SMILES: CC(C)(C)OC(=O)CCOCCNCc1cc(F)cc2c1ccn2Cc1ccc(Cl)cc1] | ¹H NMR (400 MHz, CDCl₃) 8 (ppm): 7.32- 7.24 (m, 2H), 7.10 (d, J = 3.2 Hz, 1H), 7.06-6.99 (m, 2H), 6.96 (dd, J = 10.4 Hz, 2.4 Hz, 1H), 6.81 (dd, J = 9.6 Hz, 2.0 Hz, 1H), 6.62 (dd, J = 3.2 Hz, 0.8 Hz, 1H), 5.22 (s, 2H), 4.14 (s, 2H), 3.72-3.62 (m, 4H), 2.90 (t, J = 5.2 Hz, 2H), 2.48 |
| | | | (t, J = 6.4 Hz, 2H), 1.42 |
| | | | (s, 9H). MS (ESI) m/z |
| | | | 461.5[C₂₅H₃₀ClFN₂O₃ + |
| | | | H]⁺. |
| 143 | | 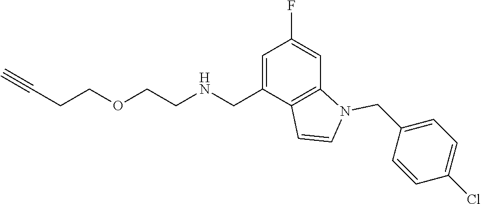 [SMILES: C#CCCOCCNCc1cc(F)cc2c1ccn2Cc1ccc(Cl)cc1] | ¹H NMR (400 MHz, CDCl₃) δ (ppm): 7.27 (d, J = 8.0 Hz, 2H), 7.09 (d, J = 3.2 Hz, 1H), 7.02 (d, J = 8.0 Hz, 2H), 6.93 (dd, J = 10.4 Hz, 2.4 Hz, 1H), 6.80 (dd, J = 9.6 Hz, 2.4 Hz, 1H), 6.61 (d, J = 3.2 Hz, 1H), 5.21 (s, 2H), 4.09 (s, 2H), 3.63 (t, J = 5.2 Hz, 2H), 3.58 (t, J = 6.8 Hz, 2H), 2.88 (t, J = 5.2 Hz, 2H), 2.46 (td, |
| | | | J = 6.8 Hz, 2.4 Hz, 2H), |
| | | | 1.97 (t, J = 2.8 Hz, 1H). |
| | | | MS (ESI) m/z 385.5 |
| | | | [C₂₂H₂₂ClFN₂O + H]⁺. |
| 144 | B | 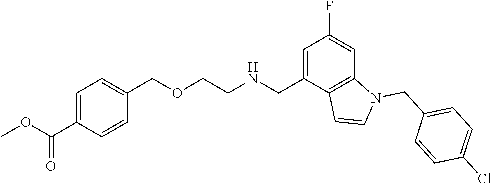 [SMILES: COC(=O)c1ccc(COCCNCc2cc(F)cc3c2ccn3Cc2ccc(Cl)cc2)cc1] | ¹H NMR (400 MHz, DMSO-d₆) δ (ppm): 7.97 (d, J = 8.0 Hz, 2H), 7.50 (dd, J = 6.0 Hz, 2.4 Hz, 3H), 7.44-7.37 (m, 2H), 7.28-7.19 (m, 3H), 6.94 (dd, J = 10.8 Hz, 2.4 Hz, 1H), 6.62 (d, J = 3.2 Hz, 1H), 5.42 (s, 2H), 4.59 (s, 2H), 4.00 (s, 2H), 3.89 (s, 3H), 3.60 (t, J = 5.6 Hz, 2H), 2.79 (t, J = 5.6 |
| | | | Hz, 2H). MS (ESI) m/z |
| | | | 481.6[C₂₇H₂₆ClFN₂O₃ + |
| | | | H]⁺. |
| 145 | B | 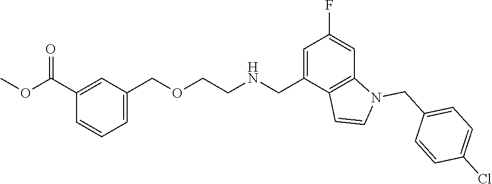 [SMILES: COC(=O)c1cccc(COCCNCc2cc(F)cc3c2ccn3Cc2ccc(Cl)cc2)c1] | ¹H NMR (400 MHz, DMSO-d₆) δ (ppm): 7.92 (s, 1H), 7.87 (d, J = 7.6 Hz, 1H), 7.60 (d, J = 7.6 Hz, 1H), 7.53-7.40 (m, 2H), 7.36 (d, J = 8.4 Hz, 2H), 7.24-7.14 (m, 3H), 6.93-6.86 (m, 1H), 6.57 (d, J = 3.2 Hz, 1H), 5.37 (s, 2H), 4.54 (s, 2H), 3.96 (s, 2H), 3.84 (s, 3H), 3.55 (t, J = 5.6 Hz, 2H), 2.75 |
| | | | (t, J = 5.6 Hz, 2H). MS |
| | | | (ESI) m/z 481.6 |
| | | | [C₂₇H₂₆ClFN₂O₃ + H]⁺. |
| 146 | B | 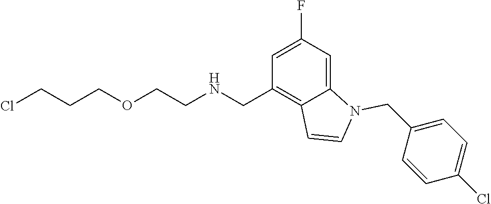 [SMILES: Fc1cc(CNCCOCCCCl)c2ccn(Cc3ccc(Cl)cc3)c2c1] | ¹H NMR (400 MHz, DMSO-d₆) δ (ppm): 9.40 (s, 2H; HCl), 7.61 (d, J = 3.2 Hz, 1H), 7.45 (d, J = 9.6 Hz, 1H), 7.38 (d, J = 8.0 Hz, 2H), 7.34-7.16 (m, 3H), 6.79 (d, J = 3.2 Hz, 1H), 5.42 (s, 2H), 4.40 (s, 2H), 3.75-3.65 (m, 4H), 3.52 (t, J = 6.0 Hz, 2H), 3.11 (t, J = 5.2 Hz, 2H), 1.96 (p, J = 6.4 Hz, 2H). MS (ESI) m/z |
| | | | 409.6[C₂₁H₂₃Cl₂FN₂O + |
| | | | H]⁺. |
| 147 | B | 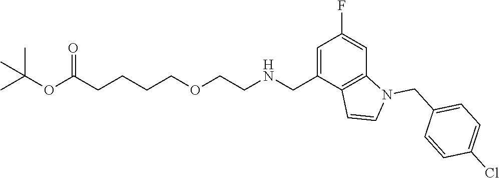 [SMILES: CC(C)(C)OC(=O)CCCCOCCNCc1cc(F)cc2c1ccn2Cc1ccc(Cl)cc1] | ¹H NMR (400 MHz, CDCl₃) δ (ppm): 7.27 (d, J = 6.4 Hz, 2H), 7.10 (d, J = 3.2 Hz, 1H), 7.05- 6.99 (m, 2H), 6.94 (dd, J = 10.4 Hz, 2.4 Hz, 1H), 6.81 (dd, J = 9.6 Hz, 2.4 Hz, 1H), 6.61 (d, J = 3.2 Hz, 1H), 5.22 (s, 2H), 4.12 (s, 2H), 3.58 (t, J = 5.2 Hz, 2H), 3.44 (t, J = |
| | | | 6.0 Hz, 2H), 2.88 (t, J = |
| | | | 5.2 Hz, 2H), 2.23 (t, J = |
| | | | 7.2 Hz, 2H), 1.70-1.52 |
| | | | (m, 4H), 1.43 (s, 9H). |
| | | | MS (ESI) m/z 489.5 |
| | | | [C₂₇H₃₄ClFN₂O₃ + H]⁺. |
| 148 | B | 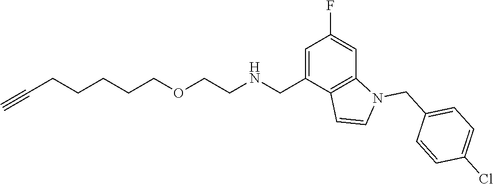 [SMILES: C#CCCCCCOCCNCc1cc(F)cc2c1ccn2Cc1ccc(Cl)cc1] | ¹H NMR (400 MHz, DMSO-d₆) δ (ppm): 7.46 (d, J = 3.2 Hz, 1H), 7.41- 7.33 (m, 2H), 7.25-7.14 (m, 3H), 6.89 (dd, J = 10.8 Hz, 2.4 Hz, 1H), 6.58 (dd, J = 3.2 Hz, 0.8 Hz, 1H), 5.38 (s, 2H), 3.95 (s, 2H), 3.44 (t, J = 5.6 Hz, 2H), 3.38-3.30 (m, 2H), 2.74-2.64 (m, 3H), 2.13 (td, J = 6.8 Hz, |
| | | | 2.8 Hz, 2H), 1.57-1.27 |
| | | | (m, 6H). MS (ESI) m/z |
| | | | 427.5[C₂₅H₂₈ClFN₂O + |
| | | | H]⁺. |
| 149 | B | 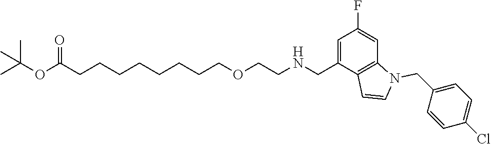 [SMILES: CC(C)(C)OC(=O)CCCCCCCCOCCNCc1cc(F)cc2c1ccn2Cc1ccc(Cl)cc1] | ¹H NMR (400 MHz, DMSO-d₆) δ (ppm): 7.46 (d, J = 3.2 Hz, 1H), 7.40- 7.33 (m, 2H), 7.24-7.15 (m, 3H), 6.89 (dd, J = 10.4 Hz, 2.4 Hz, 1H), 6.58 (d, J = 3.2 Hz, 1H), 5.38 (s, 2H), 3.95 (s, 2H), 3.43 (t, J = 5.6 Hz, |
| | | | 2H), 2.67 (t, J = 5.6 Hz, |
| | | | 2H), 2.14 (t, J = 7.2 Hz, |
| | | | 2H), 2.05-1.95 (m, 1H), |
| | | | 1.49-1.42 (m, 4H), 1.37 |
| | | | (s, 9H), 1.23 (s, 10H). |
| | | | MS (ESI) m/z 545.6 |
| | | | [C₃₁H₄₂ClFN₂O₃ + H]⁺. |
| 150 | B | 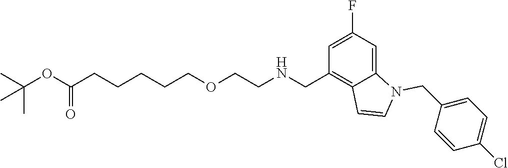 [SMILES: CC(C)(C)OC(=O)CCCCCOCCNCc1cc(F)cc2c1ccn2Cc1ccc(Cl)cc1] | ¹H NMR (400 MHz, CDCl₃) δ (ppm): 7.31- 7.24 (m, 2H), 7.11 (d, J = 3.2 Hz, 1H), 7.06-7.00 (m, 2H), 6.96 (dd, J = 10.4 Hz, 2.4 Hz, 1H), 6.82 (dd, J = 9.6 Hz, 2.4 Hz, 1H), 6.62 (dd, J = 3.2 Hz, 0.8 Hz, 1H), 5.22 (s, 2H), 4.16 (s, 2H), 3.60 (t, J = 5.2 Hz, 2H), 3.43 |
| | | | (t, J = 6.4 Hz, 2H), 2.91 |
| | | | (t, J = 5.2 Hz, 2H), 2.24- |
| | | | 2.16 (m, 2H), 1.65-1.52 |
| | | | (m, 4H), 1.43 (s, 9H), |
| | | | 1.41-1.29 (m, 2H). MS |
| | | | (ESI) m/z 503.6 |
| | | | [C₂₈H₃₆ClFN₂O₃ + H]⁺. |
| 151 | B | 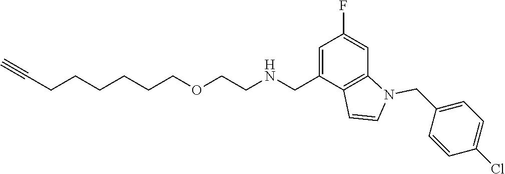 [SMILES: C#CCCCCCCOCCNCc1cc(F)cc2c1ccn2Cc1ccc(Cl)cc1] | ¹H NMR (400 MHz, DMSO-d₆) δ (ppm): 7.46 (d, J = 3.2 Hz, 1H), 7.41- 7.33 (m, 2H), 7.24-7.14 (m, 3H), 6.89 (dd, J = 10.8 Hz, 2.4 Hz, 1H), 6.58 (d, J = 3.2 Hz, 1H), 5.37 (s, 2H), 3.95 (s, 2H), 3.43 (t, J = 5.6 Hz, 2H), 3.33 (d, J = 6.4 Hz, 2H), 2.72 (t, J = 2.4 Hz, 1H), |
| | | | 2.68 (t, J = 5.6 Hz, 2H), |
| | | | 2.12 (td, J = 6.8 Hz, 2.4 |
| | | | Hz, 2H), 1.49-1.24 (m, |
| | | | 8H). MS (ESI) m/z 441.5 |
| | | | [C₂₆H₃₀ClFN₂O + H]⁺. |
| 152 | B | 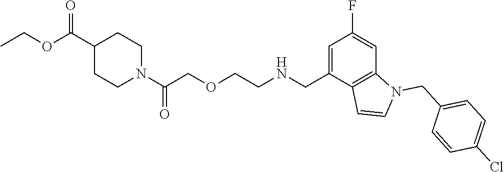 [SMILES: CCOC(=O)C1CCN(C(=O)COCCNCc2cc(F)cc3c2ccn3Cc2ccc(Cl)cc2)CC1] | ¹H NMR (400 MHz, DMSO-d₆) δ (ppm): 7.46 (d, J = 3.2 Hz, 1H), 7.37 (d, J = 8.4 Hz, 2H), 7.24- 7.14 (m, 3H), 6.89 (d, J = 10.4 Hz, 1H), 6.59 (d, J = 3.2 Hz, 1H), 5.38 (s, 2H), 4.18-4.00 (m, 5H), 3.95 (s, 2H), 3.72 (d, J = 14.0 Hz, 1H), 3.51 (t, J = 5.6 Hz, 2H), 3.03 (t, J = 12.4 |
| | | | Hz, 1H), 2.77-2.65 (m, |
| | | | 3H), 2.55 (td, J = 10.8 |
| | | | Hz, 5.6 Hz, 1H), 1.80 (d, |
| | | | J = 13.2 Hz, 2H), 1.53- |
| | | | 1.45 (m, 1H), 1.40-1.33 |
| | | | (m, 1H), 1.16 (t, J = 7.2 |
| | | | Hz, 3H). MS (ESI) m/z |
| | | | 530.7[C₂₈H₃ClFN₃O₄ + |
| | | | H]⁺. |
| 153 | B | 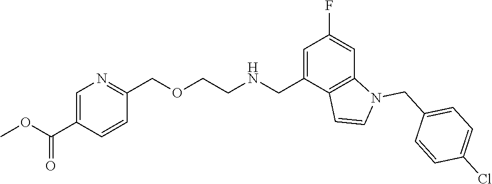 [SMILES: COC(=O)c1ccc(COCCNCc2cc(F)cc3c2ccn3Cc2ccc(Cl)cc2)nc1] | ¹H NMR (400 MHz, DMSO-d₆) δ (ppm): 9.01 (dd, J = 2.0 Hz, 0.8 Hz, 1H), 8.27 (dd, J = 8.4 Hz, 2.0 Hz, 1H), 7.61 (dd, J = 8.4 Hz, 0.8 Hz, 1H), 7.46 (d, J = 3.2 Hz, 1H), 7.40-7.33 (m, 2H), 7.25-7.14 (m, 3H), 6.91 (dd, J = 10.8 Hz, 2.4 Hz, 1H), 6.59 (dd, J = 3.2 Hz, 0.8 Hz, 1H), 5.37 (s, |
| | | | 2H), 4.64 (s, 2H), 3.97 |
| | | | (s, 2H), 3.88 (s, 3H), |
| | | | 3.63 (t, J = 5.6 Hz, 2H), |
| | | | 2.78 (t, J = 5.6 Hz, 2H). |
| | | | MS (ESI) m/z 482.6 |
| | | | [C₂₆H₂₅ClFN₃O₃ + H]⁺. |
| 154 | B | 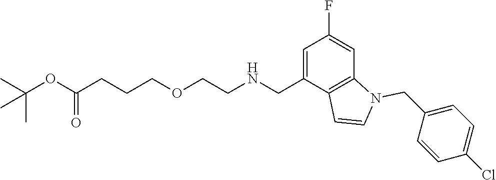 [SMILES: CC(C)(C)OC(=O)CCCOCCNCc1cc(F)cc2c1ccn2Cc1ccc(Cl)cc1] | ¹H NMR (400 MHz, CDCl₃) δ (ppm): 7.32- 7.24 (m, 2H), 7.09 (d, J = 3.2 Hz, 1H), 7.05-6.98 (m, 2H), 6.88 (dd, J = 10.4 Hz, 2.4 Hz, 1H), 6.82 (dd, J = 9.2 Hz, 2.4 Hz, 1H), 6.64 (dd, J = 3.2 Hz, 0.8 Hz, 1H), 5.22 (s, 2H), 3.89 (s, 2H), 3.55 (t, J = 5.2 Hz, 2H), 2.68 |
| | | | (t, J = 5.2 Hz, 2H), 2.60 |
| | | | (t, J = 7.2 Hz, 2H), 2.23 |
| | | | (t, J = 7.2 Hz, 2H), 1.86 |
| | | | (p, J = 7.2 Hz, 2H), 1.43 |
| | | | (s, 9H). MS (ESI) m/z |
| | | | 475.6[C₂₆H₃₂ClFN₂O₃ + |
| | | | H]⁺. |
| 155 | B | 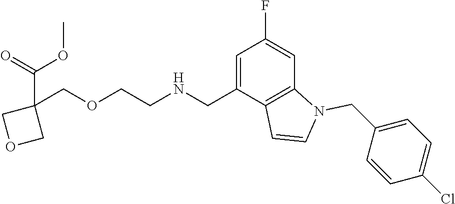 [SMILES: COC(=O)C1(COCCNCc2cc(F)cc3c2ccn3Cc2ccc(Cl)cc2)COC1] | ¹H NMR (400 MHz, CDCl₃) δ (ppm): 7.27 (d, J = 6.4 Hz, 2H), 7.10 (d, J = 3.2 Hz, 1H), 7.06- 6.98 (m, 2H), 6.92 (dd, J = 10.4 Hz, 2.4 Hz, 1H), 6.81 (dd, J = 9.6 Hz, 2.0 Hz, 1H), 6.60 (dd, J = 3.2 Hz, 0.8 Hz, 1H), 5.22 (s, 2H), 4.88 (d, J = 6.4 Hz, 2H), 4.52 (d, J = 6.4 Hz, 2H), 4.10 (s, 2H), 3.91 (s, 2H), 3.74 (s, |
| | | | 3H), 3.67 (t, J = 5.2 Hz, |
| | | | 2H), 2.89 (t, J = 5.2 Hz, |
| | | | 2H). MS (ESI) m/z 461.4 |
| | | | [C₂₄H₂₆ClFN₂O₄ + H]⁺. |
| 156 | B | 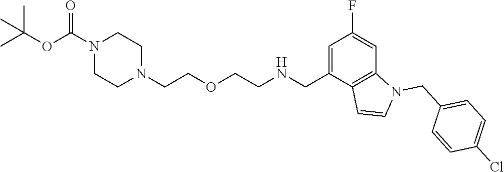 [SMILES: CC(C)(C)OC(=O)N1CCN(CCOCCNCc2cc(F)cc3c2ccn3Cc2ccc(Cl)cc2)CC1] | ¹H NMR (400 MHz, DMSO-d₆) δ (ppm): 7.43 (d, J = 3.2 Hz, 1H), 7.37- 7.31 (m, 2H), 7.21-7.11 (m, 3H), 6.89 (dd, J = 10.8 Hz, 2.4 Hz, 1H), 6.64 (d, J = 3.2 Hz, 1H), 5.35 (s, 2H), 3.82 (s, 2H), 3.45 (t, J = 6.0 Hz, 2H), 3.19 (t, J = 4.8 Hz, 4H), 2.56 (td, J = 6.4 Hz, |
| | | | 3.6 Hz, 4H), 2.35 (t, J = |
| | | | 6.4 Hz, 2H), 2.17 (t, J = |
| | | | 5.2 Hz, 4H), 1.83 (s, |
| | | | 1H), 1.35 (s, 9H). MS |
| | | | (ESI) m/z 545.8 |
| | | | [C₂₉H₃₈ClFN₄O₃ + H]⁺. |
| 157 | B | 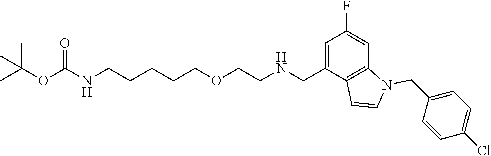 [SMILES: CC(C)(C)OC(=O)NCCCCCOCCNCc1cc(F)cc2c1ccn2Cc1ccc(Cl)cc1] | ¹H NMR (400 MHz, CDCl₃) δ (ppm): 7.27 (d, J = 6.4 Hz, 2H), 7.10 (d, J = 3.2 Hz, 1H), 7.06- 6.99 (m, 2H), 6.93 (dd, J = 10.4 Hz, 2.4 Hz, 1H), 6.80 (dd, J = 9.6 Hz, 2.4 Hz, 1H), 6.60 (dd, J = 3.2 Hz, 0.8 Hz, 1H), 5.22 (s, 2H), 4.53 (s, 1H), 4.10 (s, |
| | | | 2H), 3.57 (t, J = 5.2 Hz, |
| | | | 2H), 3.42 (t, J = 6.4 Hz, |
| | | | 2H), 3.09 (q, J = 6.4 Hz, |
| | | | 2H), 2.87 (t, J = 5.2 Hz, |
| | | | 2H), 1.63-1.42 (m, 4H), |
| | | | 1.43 (s, 9H), 1.41-1.29 |
| | | | (m, 2H). MS (ESI) m/z |
| | | | 518.7[C₂₈H₃₇ClFN₃O₃ + |
| | | | H]⁺. |
| 158 | B | 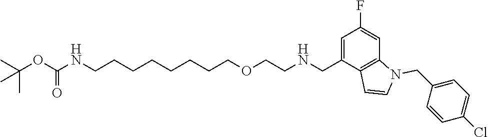 [SMILES: CC(C)(C)OC(=O)NCCCCCCCCOCCNCc1cc(F)cc2c1ccn2Cc1ccc(Cl)cc1] | ¹H NMR (400 MHz, CDCl₃) δ (ppm): 7.32- 7.23 (m, 2H), 7.09 (d, J = 3.2 Hz, 1H), 7.06-6.99 (m, 2H), 6.93 (dd, J = 10.4 Hz, 2.4 Hz, 1H), 6.84-6.76 (m, 1H), 6.60 (dd, J = 3.2 Hz, 0.8 Hz, 1H), 5.22 (s, 2H), 4.50 |
| | | | (s, 1H), 4.10 (s, 2H), |
| | | | 3.61-3.54 (m, 2H), 3.41 |
| | | | (t, J = 6.8 Hz, 2H), 3.08 |
| | | | (q, J = 6.8 Hz, 2H), 2.91- |
| | | | 2.84 (m, 2H), 1.54 (q, |
| | | | J = 6.8 Hz, 2H), 1.44 (s, |
| | | | 11H), 1.30-1.26 (m, 8H). |
| | | | MS (ESI) m/z 560.7 |
| | | | [C₃₁H₄₃ClFN₃O₃ + H]⁺. |
| 159 | B | 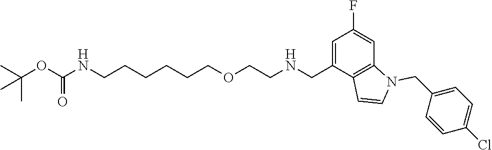 [SMILES: CC(C)(C)OC(=O)NCCCCCCOCCNCc1cc(F)cc2c1ccn2Cc1ccc(Cl)cc1] | ¹H NMR (400 MHz, CDCl₃) δ (ppm): 7.27 (d, J = 6.4 Hz, 2H), 7.10 (d, J = 3.2 Hz, 1H), 7.05- 6.98 (m, 2H), 6.94 (dd, J = 10.4 Hz, 2.4 Hz, 1H), 6.81 (dd, J = 9.6 Hz, 2.4 Hz, 1H), 6.61 (dd, J = 3.2 Hz, 0.8 Hz, 1H), |
| | | | 5.22 (s, 2H), 4.50 (s, |
| | | | 1H), 4.12 (s, 2H), 3.57 |
| | | | (t, J = 5.2 Hz, 2H), 3.42 |
| | | | (t, J = 6.8 Hz, 2H), 3.09 |
| | | | (q, J = 6.8 Hz, 2H), 2.89 |
| | | | (t, J = 5.2 Hz, 2H), 1.60- |
| | | | 1.43 (m, 4H), 1.44 (s, |
| | | | 9H), 1.38-1.27 (m, 4H). |
| | | | MS (ESI) m/z 532.6 |
| | | | [C₂₉H₃₉ClFN₃O₃ + H]⁺. |
| 160 | B | 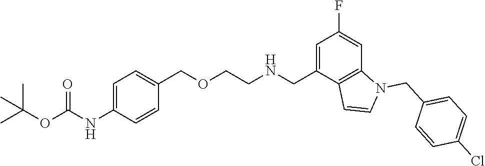 [SMILES: CC(C)(C)OC(=O)Nc1ccc(COCCNCc2cc(F)cc3c2ccn3Cc2ccc(Cl)cc2)cc1] | ¹H NMR (400 MHz, CDCl₃) δ (ppm): 7.33 (d, J = 8.0 Hz, 2H), 7.29- 7.22 (m, 3H), 7.06 (d, J = 3.2 Hz, 1H), 7.04-6.96 (m, 2H), 6.88 (dd, J = 10.4 Hz, 2.4 Hz, 1H), 6.80 (dd, J = 9.6 Hz, 2.4 Hz, 1H), 6.53 (dd, J = 3.2 Hz, 0.8 Hz, 1H), 6.49 |
| | | | (s, 1H), 5.21 (s, 2H), 3.84 |
| | | | (s, 2H), 3.63 (s, 2H), 3.55 |
| | | | (t, J = 5.2 Hz, 2H), 2.66 |
| | | | (t, J = 5.2 Hz, 2H), 1.52 |
| | | | (s, 9H). MS (ESI) m/z |
| | | | 538.7[C₃₀H₃₃ClFN₃O₃ + |
| | | | H]⁺. |
| 161 | B | 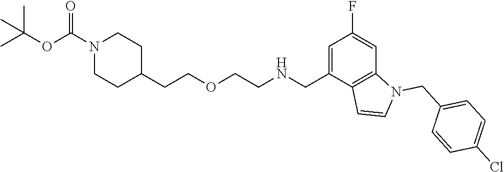 [SMILES: CC(C)(C)OC(=O)N1CCC(CCOCCNCc2cc(F)cc3c2ccn3Cc2ccc(Cl)cc2)CC1] | ¹H NMR (400 MHz, DMSO-d₆) δ (ppm): 7.46 (d, J = 3.2 Hz, 1H), 7.39- 7.35 (m, 2H), 7.24-7.15 (m, 3H), 6.89 (dd, J = 10.8 Hz, 2.4 Hz, 1H), 6.58 (d, J = 3.2 Hz, 1H), 5.37 (s, 2H), 3.95 (s, 2H), 3.87 (d, J = 13.2 Hz, 2H), 3.47-3.34 (m, 4H), 2.68 (t, J = 5.6 Hz, 2H), 2.61 |
| | | | (s, 2H), 1.58 (dd, J = |
| | | | 12.8 Hz, 3.6 Hz, 2H), |
| | | | 1.54-1.44 (m, 1H), 1.41 |
| | | | (t, J = 6.4 Hz, 2H), 1.37 |
| | | | (s, 9H), 0.94 (qd, J = |
| | | | 12.4 Hz, 4.4 Hz, 2H). |
| | | | MS (ESI) m/z 544.9 |
| | | | [C₃₀H₃₉ClFN₃O₃ + H]⁺. |
| 162 | B | 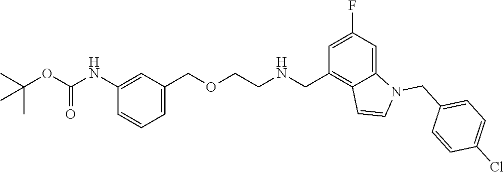 [SMILES: CC(C)(C)OC(=O)Nc1cccc(COCCNCc2cc(F)cc3c2ccn3Cc2ccc(Cl)cc2)c1] | ¹H NMR (400 MHz, CDCl₃) δ (ppm): 7.34 (s, 1H), 7.31-7.20 (m, 3H), 7.08 (d, J = 3.2 Hz, 1H), 7.05-6.96 (m, 3H), 6.93 (dd, J = 10.4 Hz, 2.4 Hz, 1H), 6.80 (dd, J = 9.6 Hz, 2.4 Hz, 1H), 6.60 (dd, J = 3.2 Hz, 0.8 Hz, 1H), 6.49 (s, 1H), 5.22 (s, 2H), 4.49 (s, 2H), 4.09 (s, 2H), 3.64 |
| | | | (t, J = 5.2 Hz, 2H), 2.91 |
| | | | (t, J = 5.2 Hz, 2H), 1.51 |
| | | | (s, 9H). MS (ESI) m/z |
| | | | 538.6[C₃₀H₃₃ClFN₃O₃ + |
| | | | H]⁺. |
| 163 | B | 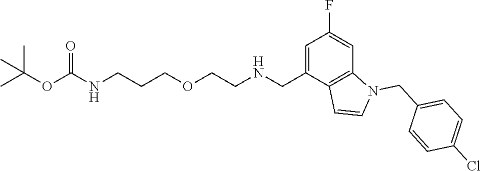 [SMILES: CC(C)(C)OC(=O)NCCCOCCNCc1cc(F)cc2c1ccn2Cc1ccc(Cl)cc1] | ¹H NMR (400 MHz, CDCl₃) δ (ppm): 7.27 (d, J = 6.8 Hz, 2H), 7.10 (d, J = 2.8 Hz, 1H), 7.06- 6.99 (m, 2H), 6.94 (d, J = 10.4 Hz, 1H), 6.80 (d, J = 9.6 Hz, 1H), 6.61 (s, 1H), 5.22 (s, 2H), 4.91 (s, 1H), 4.12 (s, 2H), 3.58 (s, 2H), 3.50 (s, 2H), 3.22 (s, 2H), 2.87 (s, 2H), 1.43 (s, 9H). |
| | | | MS (ESI) m/z 490.5 |
| | | | [C₂₆H₃₃ClFN₃O₃ + H]⁺. |
| 164 | B | 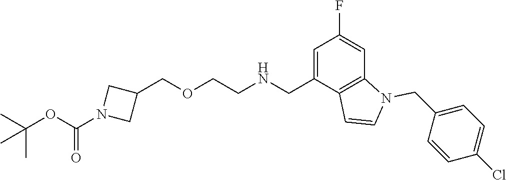 [SMILES: CC(C)(C)OC(=O)N1CC(COCCNCc2cc(F)cc3c2ccn3Cc2ccc(Cl)cc2)C1] | ¹H NMR (400 MHz, DMSO-d₆) δ (ppm): 7.46 (d, J = 3.2 Hz, 1H), 7.41- 7.33 (m, 2H), 7.25-7.14 (m, 3H), 6.89 (dd, J = 10.8 Hz, 2.4 Hz, 1H), 6.58 (dd, J = 3.2 Hz, 0.8 Hz, 1H), 5.37 (s, 2H), 3.95 (s, 2H), 3.84 (s, 2H), 3.54-3.46 (m, 6H), 2.69 (t, J = 5.6 Hz, 3H), 1.34 |
| | | | (s, 9H). MS (ESI) m/z |
| | | | 502.8[C₂₇H₃₃ClFN₃O₃ + |
| | | | H]⁺. |
| 165 | B | 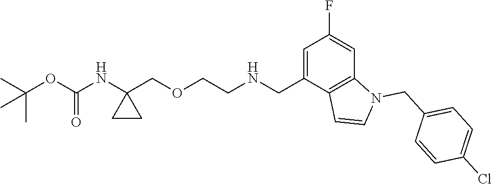 [SMILES: CC(C)(C)OC(=O)NC1(COCCNCc2cc(F)cc3c2ccn3Cc2ccc(Cl)cc2)CC1] | ¹H NMR (400 MHz, CDCl₃) δ (ppm): 7.28 (d, J = 2.0 Hz, 2H), 7.10 (d, J = 3.2 Hz, 1H), 7.02 (d, J = 8.0 Hz, 2H), 6.94 (dd, J = 10.4 Hz, 2.4 Hz, 1H), 6.81 (dd, J = 9.6 Hz, 2.4 Hz, 1H), 6.61 (d, J = 3.2 Hz, 1H), 5.22 (s, 2H), 5.12 (s, 1H), 4.11 (s, 2H), 3.63 (t, J = 5.2 Hz, 2H), |
| | | | 3.45 (s, 2H), 2.88 (t, J = |
| | | | 5.2 Hz, 2H), 1.41 (s, 9H), |
| | | | 0.87-0.64 (m, 4H). MS |
| | | | (ESI) m/z 502.7 |
| | | | [C₂₇H₃₃ClFN₃O₃ + H]⁺. |
| 166 | B | 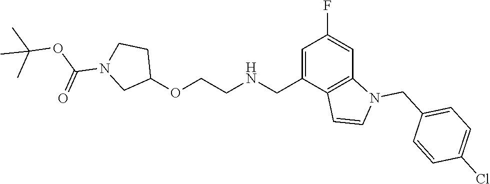 [SMILES: CC(C)(C)OC(=O)N1CCC(OCCNCc2cc(F)cc3c2ccn3Cc2ccc(Cl)cc2)C1] | ¹H NMR (400 MHz, CDCl₃) δ (ppm): 7.32- 7.24 (m, 2H), 7.11 (d, J = 3.2 Hz, 1H), 7.06-6.99 (m, 2H), 6.92 (dd, J = 10.4 Hz, 2.4 Hz, 1H), 6.82 (dd, J = 9.6 Hz, 2.4 Hz, 1H), 6.60 (dd, J = 3.2 Hz, 0.8 Hz, 1H), 5.22 (s, 2H), 4.12 (s, 2H), 4.02 (s, 1H), 3.63-3.56 (m, 2H), 3.43-3.35 (m, 4H), |
| | | | 2.89 (t, J = 5.2 Hz, 2H), |
| | | | 2.05 (s, 1H), 2.01-1.82 |
| | | | (m, 2H), 1.45 (s, 9H). |
| | | | MS (ESI) m/z 502.6 |
| | | | [C₂₇H₃₃ClFN₃O₃ + H]⁺. |
| 167 | B | 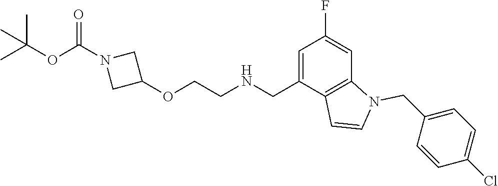 [SMILES: CC(C)(C)OC(=O)N1CC(OCCNCc2cc(F)cc3c2ccn3Cc2ccc(Cl)cc2)C1] | ¹H NMR (400 MHz, DMSO-d₆) δ (ppm): 7.46 (d, J = 3.2 Hz, 1H), 7.40-7.33 (m, 2H), 7.24- 7.14 (m, 3H), 6.90 (dd, J = 10.8 Hz, 2.4 Hz, 1H), 6.59 (d, J = 3.2 Hz, 1H), 5.38 (s, 2H), 4.26-4.16 (m, 1H), 4.01-3.92 (m, 4H), 3.63 (s, 2H), 3.41 (t, J = 5.6 Hz, 2H), 2.68 (t, J = 5.6 Hz, 2H), 1.36 |
| | | | (s, 9H). MS (ESI) m/z |
| | | | 488.7[C₂₆H₃₁ClFN₃O₃ + |
| | | | H]⁺. |
| 168 | B | 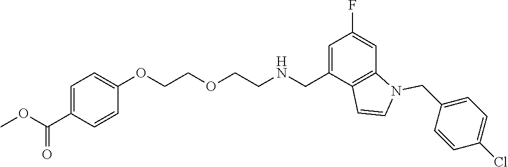 [SMILES: COC(=O)c1ccc(OCCOCCNCc2cc(F)cc3c2ccn3Cc2ccc(Cl)cc2)cc1] | ¹H NMR (400 MHz, CDCl₃) δ (ppm): 8.00- 7.92 (m, 2H), 7.30-7.23 (m, 2H), 7.05 (d, J = 3.2 Hz, 1H), 7.04-6.98 (m, 2H), 6.96-6.86 (m, 3H), 6.80 (dd, J = 9.6 Hz, 2.4 Hz, 1H), 6.59 (dd, J = 3.2 Hz, 0.8 Hz, 1H), 5.20 (s, 2H), 4.16 (dd, J = 5.6 Hz, 3.6 Hz, 2H), |
| | | | 4.10 (s, 2H), 3.88 (s, |
| | | | 3H), 3.86-3.80 (m, 2H), |
| | | | 3.72 (t, J = 5.2 Hz, 2H), |
| | | | 2.92 (t, J = 5.2 Hz, 2H). |
| | | | MS (ESI) m/z 511.5 |
| | | | [C₂₈H₂₈ClFN₂O₄ + H]⁺. |
| 169 | B | 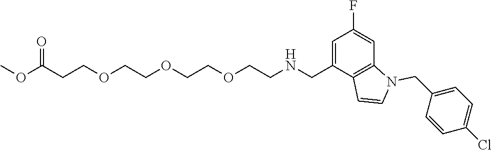 [SMILES: COC(=O)CCOCCOCCOCCNCc1cc(F)cc2c1ccn2Cc1ccc(Cl)cc1] | ¹H NMR (400 MHz, CDCl₃) δ (ppm): 7.31- 7.23 (m, 2H), 7.09 (d, J = 3.2 Hz, 1H), 7.06-6.98 (m, 2H), 6.93 (dd, J = 10.4 Hz, 2.4 Hz, 1H), 6.79 (dd, J = 9.6 Hz, 2.4 Hz, 1H), 6.60 (dd, J = 3.2 Hz, 0.8 Hz, 1H), 5.22 (s, 2H), 4.09 (s, |
| | | | 2H), 3.73 (t, J = 6.4 Hz, |
| | | | 2H), 3.67 (s, 3H), 3.66- |
| | | | 3.56 (m, 10H), 2.88 (t, |
| | | | J = 5.2 Hz, 2H), 2.58 (t, |
| | | | J = 6.4 Hz, 2H). MS |
| | | | (ESI) m/z 507.5 |
| | | | [C₂₆H₃₂ClFN₂O₅ + H]⁺. |
| 170 | B | 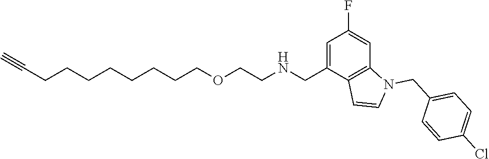 [SMILES: C#CCCCCCCCCOCCNCc1cc(F)cc2c1ccn2Cc1ccc(Cl)cc1] | ¹H NMR (400 MHz, CDCl₃) δ (ppm): 7.31- 7.23 (m, 2H), 7.09 (d, J = 3.2 Hz, 1H), 7.06- 6.98 (m, 2H), 6.93 (dd, J = 10.4 Hz, 2.4 Hz, 1H), 6.84-6.76 (m, 1H), 6.60 (dd, J = 3.2 Hz, 0.8 Hz, 1H), 5.22 (s, 2H), 4.10 (s, 2H), 3.61-3.54 (m, |
| | | | 2H), 3.42 (t, J = 6.8 Hz, |
| | | | 2H), 2.91-2.84 (m, 2H), |
| | | | 2.17 (td, J = 7.2, 2.4 Hz, |
| | | | 2H), 1.94 (s, 1H), 1.62- |
| | | | 1.45 (m, 4H), 1.44-1.24 |
| | | | (m, 8H). MS (ESI) m/z |
| | | | 469.5[C₂₈H₃₄ClFN₂O + |
| | | | H]⁺. |
| 171 | B | 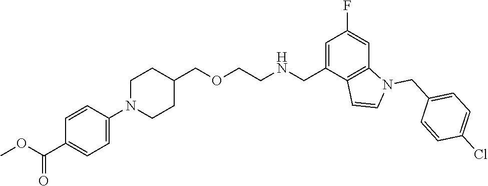 [SMILES: COC(=O)c1ccc(N2CCC(COCCNCc3cc(F)cc4c3ccn4Cc3ccc(Cl)cc3)CC2)cc1] | ¹H NMR (400 MHz, CDCl₃) δ (ppm): 7.93- 7.85 (m, 2H), 7.27 (d, J = 8.0 Hz, 2H), 7.08 (d, J = 3.2 Hz, 1H), 7.05-6.98 (m, 2H), 6.94 (dd, J = 10.4 Hz, 2.4 Hz, 1H), 6.90-6.78 (m, 3H), 6.60 (d, J = 3.2 Hz, 1H), 5.21 (s, 2H), 4.12 (s, 2H), 3.86 (s, 5H), 3.60 (t, J = 5.2 Hz, 2H), 3.30 (d, J = |
| | | | 6.0 Hz, 2H), 2.89 (t, J = |
| | | | 5.2 Hz, 2H), 2.82 (td, |
| | | | J = 12.8 Hz, 2.4 Hz, 2H), |
| | | | 1.81 (dt, J = 11.6 Hz, 3.2 |
| | | | Hz, 3H), 1.38-1.29 (m, |
| | | | 2H). MS (ESI) m/z 564.6 |
| | | | [C₃₂H₃₅ClFN₃O₃ + H]⁺. |
| 172 | B | 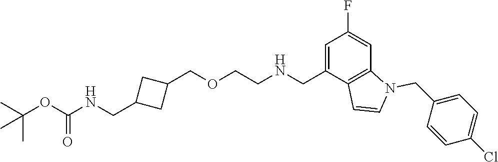 [SMILES: CC(C)(C)OC(=O)NCC1CC(COCCNCc2cc(F)cc3c2ccn3Cc2ccc(Cl)cc2)C1] | ¹H NMR (400 MHz, CDCl₃) δ (ppm): 7.31- 7.23 (m, 2H), 7.12-7.06 (m, 1H), 7.06-6.99 (m, 2H), 6.97-6.88 (m, 1H), 6.80 (dd, J = 9.6 Hz, 2.4 Hz, 1H), 6.60 (d, J = 3.2 Hz, 1H), 5.22 (s, 2H), 4.52 (d, J = 6.0 Hz, 1H), 4.11 (s, 2H), 3.62-3.53 |
| | | | (m, 2H), 3.35 (d, J = 6.0 |
| | | | Hz, 2H), 3.07 (t, J = 6.4 |
| | | | Hz, 2H), 2.87 (t, J = 5.2 |
| | | | Hz, 2H), 2.50-2.23 (m, |
| | | | 2H), 2.17-2.05 (m, 2H), |
| | | | 1.43 (s, 11H). MS |
| | | | (ESI) m/z 530.6 |
| | | | [C₂₉H₃₇ClFN₃O₃ + H]⁺. |
| 173 | B | 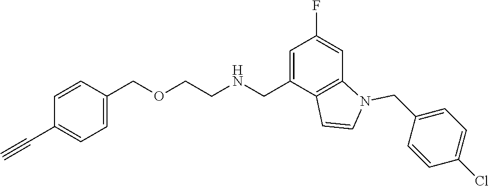 [SMILES: C#Cc1ccc(COCCNCc2cc(F)cc3c2ccn3Cc2ccc(Cl)cc2)cc1] | ¹H NMR (400 MHz, DMSO-d₆) δ (ppm): 7.49-7.41 (m, 3H), 7.41- 7.29 (m, 4H), 7.25-7.14 (m, 3H), 6.90 (dd, J = 10.8 Hz, 2.4 Hz, 1H), 6.58 (dd, J = 3.2 Hz, 0.8 Hz, 1H), 5.38 (s, 2H), 4.48 (s, 2H), 4.16 (s, 1H), 3.95 (s, 2H), 3.53 (t, J = 5.6 Hz, 2H), 2.73 (t, J = 5.6 Hz, 2H). MS |
| | | | (ESI) m/z 447.5 |
| | | | [C₂₇H₂₄ClFN₂O + H]⁺. |
| 174 | B | 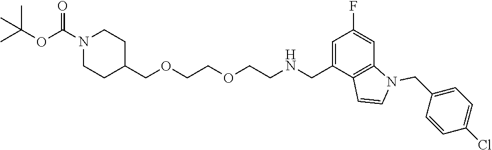 [SMILES: CC(C)(C)OC(=O)N1CCC(COCCOCCNCc2cc(F)cc3c2ccn3Cc2ccc(Cl)cc2)CC1] | ¹H NMR (400 MHz, DMSO-d₆) δ (ppm): 7.46 (d, J = 3.2 Hz, 1H), 7.40- 7.33 (m, 2H), 7.24-7.14 (m, 3H), 6.89 (dd, J = 10.8 Hz, 2.4 Hz, 1H), 6.58 (d, J = 3.2 Hz, 1H), 5.38 (s, 2H), 3.95 (s, 2H), 3.89 (d, J = 12.8 Hz, 2H), |
| | | | 3.53-3.42 (m, 6H), 3.21 |
| | | | (d, J = 6.0 Hz, 2H), 2.69 |
| | | | (t, J = 5.6 Hz, 2H), 2.62 |
| | | | (s, 2H), 1.60 (t, J = 14.8 |
| | | | Hz, 2H), 1.37 (s, 9H), |
| | | | 1.24-1.22 (m, 1H), 0.96 |
| | | | (qd, J = 12.4 Hz, 4.4 Hz, |
| | | | 2H). MS (ESI) m/z 574.7 |
| | | | [C₃₁H₄₁ClFN₃O₄ + H]⁺. |
| 175 | B | 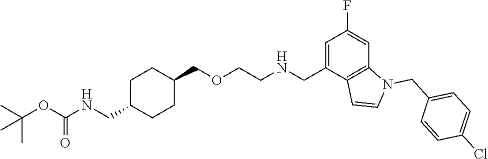 [SMILES: CC(C)(C)OC(=O)NC[C@H]1CC[C@H](COCCNCc2cc(F)cc3c2ccn3Cc2ccc(Cl)cc2)CC1] | ¹H NMR (400 MHz, CDCl₃) δ (ppm): 7.31- 7.23 (m, 2H), 7.08 (d, J = 3.2 Hz, 1H), 7.05- 6.98 (m, 2H), 6.92 (dd, J = 10.4 Hz, 2.4 Hz, 1H), 6.80 (dd, J = 9.6 Hz, 2.4 Hz, 1H), 6.59 (dd, J = 3.2 Hz, 0.8 Hz, 1H), 5.22 (s, 2H), 4.58 (s, 1H), |
| | | | 4.09 (s, 2H), 3.55 (t, J = |
| | | | 5.2 Hz, 2H), 3.23 (d, J = |
| | | | 6.4 Hz, 2H), 2.96 (t, J = |
| | | | 6.4 Hz, 2H), 2.86 (t, J = |
| | | | 5.2 Hz, 2H), 1.82-1.72 |
| | | | (m, 4H), 1.57-1.47 (m, |
| | | | 1H), 1.44 (s, 9H), 1.37 |
| | | | (s, 1H), 1.00-0.84 (m, |
| | | | 4H). MS (ESI) m/z 558.7 |
| | | | [C₃₁H₄₁ClFN₃O₃ + H]⁺. |
| 176 | B | 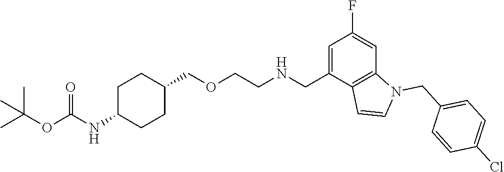 [SMILES: CC(C)(C)OC(=O)N[C@H]1CC[C@@H](COCCNCc2cc(F)cc3c2ccn3Cc2ccc(Cl)cc2)CC1] | ¹H NMR (400 MHz, CDCl₃) δ (ppm): 7.31- 7.23 (m, 2H), 7.09 (d, J = 3.2 Hz, 1H), 7.06- 6.98 (m, 2H), 6.93 (dd, J = 10.4 Hz, 2.4 Hz, 1H), 6.80 (dd, J = 9.6 Hz, 2.4 Hz, 1H), 6.60 (dd, J = 3.2 Hz, 0.8 Hz, 1H), 5.22 (s, 2H), 4.63 (s, 1H), 4.10 (s, 2H), 3.73 (s, 1H), |
| | | | 3.60-3.53 (m, 2H), 3.28 |
| | | | (d, J = 6.4 Hz, 2H), 2.87 |
| | | | (t, J = 5.2 Hz, 2H), 1.84- |
| | | | 1.48 (m, 7H), 1.44 (s, |
| | | | 9H), 1.29-1.14 (m, 2H). |
| | | | MS (ESI) m/z 544.7 |
| | | | [C₃₀H₃₉ClFN₃O₃ + H]⁺. |

TABLE 6
Summary of compounds (series III) prepared according to the examples above
and characterizations thereof

| Cpd | Synthesis | | NMR and Mass |
|---|---|---|---|
| No. | Route | Structure | Spectrometry |
| 97 | K | 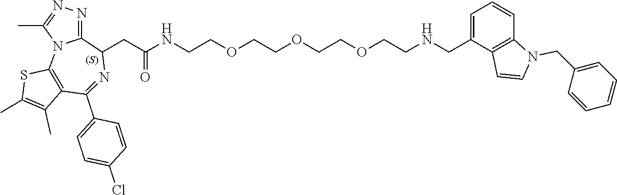 | ¹H NMR (400 MHz, DMSO-d₆) δ (ppm): 8.27 (t, 1H), 8.24 (s, 1H; HCOOH), 7.49- 7.47 (m, 3H), 7.43-7.41 (m, 2H), 7.32-7.24 (m, 4H), 7.19-7.17 (m, 2H), 7.05-6.98 (m, 2H), 6.58 (dd, J = 3.2 Hz, 0.8 Hz, 1H), |
| | | | 5.40 (s, 2H), 4.52- |
| | | | 4.49 (t→dd, 1H), |
| | | | 3.97 (s, 2H), 3.53- |
| | | | 3.48 (m, 10H), |
| | | | 3.45-3.42 (m, 2H), |
| | | | 3.28-3.17 (m, 4H), |
| | | | 2.72 (t, J = 5.6 Hz, |
| | | | 2H), 2.59 (s, 3H), |
| | | | 2.40 (s, 3H), 1.61 |
| | | | (s, 3H). |
| | | | MS (ESI) m/z |
| | | | 794.4 |
| | | | [C₄₃H₄₈ClN₇O₄S + |
| | | | H]⁺. |
| 98 | K | 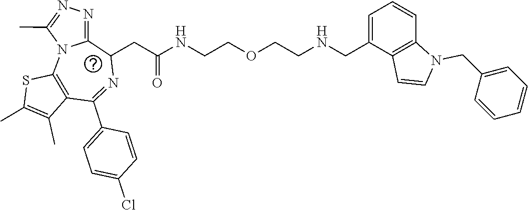 [SMILES: Cc1sc2c(c1C)C(c1ccc(Cl)cc1)=NC(CC(=O)NCCOCCNCc1cccc3c1ccn3Cc1ccccc1)c1nnc(C)n1-2] | ¹H NMR (400 MHz, DMSO-d₆) δ (ppm): 8.29 (t, J = 5.2 Hz, 1H) 8.21 (s, 1H; HCOOH), 7.51-7.47 (m, 3H), 7.43-7.41 (m, 2H), 7.38-7.35 (m, 1H), 7.30-7.27 (m, 2H), 7.24-7.17 (m, 3H), 7.07-7.05 (m, 2H), 6.64 (d, J = 3.2 Hz, 1H), 5.41 (s, 2H), |
| | | | 4.52-4.49 (m, 1H), |
| | | | 4.11 (s, 2H), 3.57 |
| | | | (t, J = 5.6 Hz, 2H), |
| | | | 3.46 (t, J = 5.6 Hz, |
| | | | 2H), 3.34-3.19 (m, |
| | | | 4H), 2.86 (t, J = |
| | | | 5.6 Hz, 2H), 2.59 |
| | | | (s, 3H), 2.40 (s, |
| | | | 3H), 1.61 (s, 3H). |
| | | | MS (ESI) m/z |
| | | | 706.3 |
| | | | [C₃₉H₄₀ClN₇O₂S + |
| | | | H]⁺. |
| 99 | K | 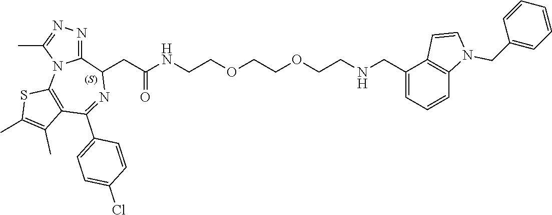 | ¹H NMR (400 MHz, DMSO-d₆) δ (ppm): 8.28 (t, J = 5.6 Hz, 1H), 8.20 (s, 1H; HCOOH), 7.53 (d, J = 3.2 Hz, 1H), 7.49-7.46 (m, 2H), 7.43-7.38 (m, 3H), 7.31-7.27 (m, 2H), 7.24-7.22 (m, 1H), 7.20-7.18 (m, 2H), 7.08-7.06 (m, 2H), 6.65 (d, J = 3.2 Hz, 1H), 5.42 |
| | | | (s, 2H), 4.52-4.49 |
| | | | (m, 1H), 4.14 (s, |
| | | | 2H), 3.57 (t, J = |
| | | | 5.6 Hz, 2H), 3.52 |
| | | | (s, 4H), 3.45 (t, J = |
| | | | 5.6 Hz, 2H), 3.30- |
| | | | 3.20 (m, 4H), 2.89 |
| | | | (t, J = 5.6 Hz, 2H), |
| | | | 2.58 (s, 3H), 2.40 |
| | | | (s, 3H), 1.61 (s, |
| | | | 3H). |
| | | | MS (ESI) m/z |
| | | | 750.4 |
| | | | [C₄₁H₄₄ClN₇O₃S + |
| | | | H]⁺. |
| 100 | K | 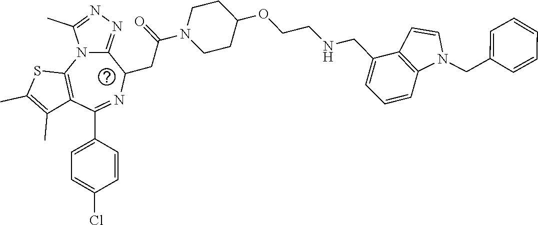 [SMILES: Cc1sc2c(c1C)C(c1ccc(Cl)cc1)=NC(CC(=O)N1CCC(OCCNCc3cccc4c3ccn4Cc3ccccc3)CC1)c1nnc(C)n1-2] | ¹H NMR (400 MHz, DMSO-d₆) δ (ppm): 8.17 (s, 1H; HCOOH), 7.55- 7.52 (m, 1H), 7.50-7.42 (m, 4H), 7.40-7.38 (m, 1H), 7.31-7.28 (m, 2H), 7.25-7.19 (m, 3H), 7.11-7.06 (m, 2H), 6.65 (t, 1H), 5.42 (s, 2H), 4.57 (t, J = 6.4 Hz, 1H), 4.12 (d, J = 3.2 Hz, 2H), |
| | | | 3.85-3.82 (m, 2H), |
| | | | 3.58-3.54 (m, 4H), |
| | | | 3.35-3.34 (m, 1H), |
| | | | 3.14-3.09 (m, 1H), |
| | | | 2.87-2.85 (m, 2H), |
| | | | 2.59 (s, 3H), 2.41 |
| | | | (s, 3H), 1.91 (br. s, |
| | | | 1H), 1.75 (br. s, |
| | | | 1H), 1.63 (s, 3H), |
| | | | 1.51 (br. s, 1H), |
| | | | 1.34 (br. s, 1H). |
| | | | MS (ESI) m/z |
| | | | 746.3 |
| | | | [C₄₂H₄₄ClN₇O₂S + |
| | | | H]⁺. |
| 101 | K | 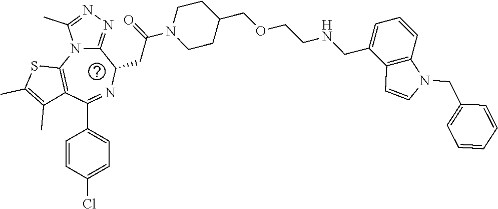 [SMILES: Cc1sc2c(c1C)C(c1ccc(Cl)cc1)=N[C@@H](CC(=O)N1CCC(COCCNCc3cccc4c3ccn4Cc3ccccc3)CC1)c1nnc(C)n1-2] | ¹H NMR (400 MHz, DMSO-d₆) δ (ppm): 7.50-7.47 (m, 3H), 7.44-7.42 (m, 2H), 7.32-7.27 (m, 3H), 7.24-7.22 (m, 1H), 7.20-7.17 (m, 2H), 7.05-7.01 (m, 1H), 6.99-6.98 (m, 1H), 6.58 (d, J = 3.2 Hz, 1H), 5.40 (s, 2H), 4.57 (t, J = 7.2 Hz, 1H), 4.36- |
| | | | 4.33 (m, 1H), |
| | | | 4.13-4.09 (m, 1H), |
| | | | 3.97 (s, 2H), 3.60- |
| | | | 3.54 (m, 1H), |
| | | | 3.47-3.46 (m, 2H), |
| | | | 3.40-3.37 (m, 2H), |
| | | | 3.25-3.22 (m, 2H), |
| | | | 3.12-3.06 (m, 1H), |
| | | | 2.71-2.70 (m, 2H), |
| | | | 2.59 (s, 3H), 2.41 |
| | | | (s, 3H), 1.79-1.73 |
| | | | (m, 2H), 1.63-1.62 |
| | | | (m, 4H), 1.24-1.16 |
| | | | (m, 1H), 0.97-0.94 |
| | | | (m, 1H). |
| | | | MS (ESI) m/z |
| | | | 760.3 |
| | | | [C₄₃H₄₆ClN₇O₂S + |
| | | | H]⁺. |
| 102 | K | 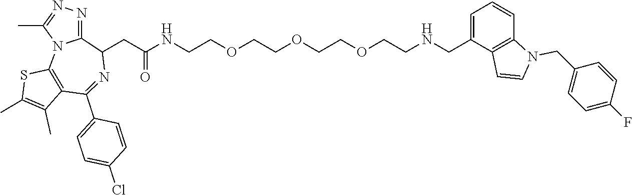 [SMILES: Cc1sc2c(c1C)C(c1ccc(Cl)cc1)=NC(CC(=O)NCCOCCOCCOCCNCc1cccc3c1ccn3Cc1ccc(F)cc1)c1nnc(C)n1-2] | ¹H NMR (400 MHz, DMSO-d₆) δ (ppm): 8.29 (t, J = 5.6 Hz, 1H), 8.25 (s, 1H; HCOOH), 7.53 (d, J = 3.2 Hz, 1H), 7.49-7.47 (m, 2H), 7.43-7.39 (m, 3H), 7.27-7.23 (m, 2H), 7.15-7.06 (m, 4H), 6.64 (d, J = 2.8 Hz, 1H), 5.41 |
| | | | (s, 2H), 4.53-4.50 |
| | | | (m, 1H), 4.11 (s, |
| | | | 2H), 3.58-3.55 (m, |
| | | | 10H), 3.44 (t, J = |
| | | | 5.6 Hz, 2H), 3.32- |
| | | | 3.19 (m, 4H), 2.85 |
| | | | (t, J = 5.6 Hz, 2H), |
| | | | 2.59 (s, 3H), 2.40 |
| | | | (s, 3H), 1.61 (s, |
| | | | 3H). |
| | | | MS (ESI) m/z |
| | | | 812.3 |
| | | | [C₄₃H₄₇ClFN₇O₄S + |
| | | | H]⁺. |
| 103 | K | 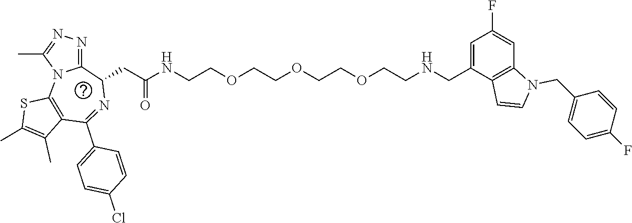 [SMILES: Cc1sc2c(c1C)C(c1ccc(Cl)cc1)=N[C@@H](CC(=O)NCCOCCOCCOCCNCc1cc(F)cc3c1ccn3Cc1ccc(F)cc1)c1nnc(C)n1-2] | ¹H NMR (400 MHz, DMSO-d₆) δ (ppm): 8.27 (t, J = 5.6 Hz, 1H), 8.19 (s, 1H; HCOOH), 7.48-7.46 (m, 3H), 7.43-7.40 (m, 2H), 7.28-7.21 (m, 3H), 7.13 (t, J = 8.8 Hz, 2H), 6.90 (dd, J = 10.8 Hz, 2.0 Hz, 1H), 6.58 (d, J = 3.2 Hz, 1H), 5.36 (s, 2H), 4.52-4.49 |
| | | | (m, 1H), 3.97 (s, |
| | | | 2H), 3.52-3.48 (m, |
| | | | 11H), 3.45-3.42 |
| | | | (m, 2H), 3.28-3.17 |
| | | | (m, 3H), 2.71 (t, J = |
| | | | 5.6 Hz, 2H), 2.58 |
| | | | (s, 3H), 2.40 (s, |
| | | | 3H), 1.61 (s, 3H). |
| | | | MS (ESI) m/z |
| | | | 830.2 |
| | | | [C₄₃H₄₆ClF₂N₇O₄S + |
| | | | H]⁺. |
| 104 | K | 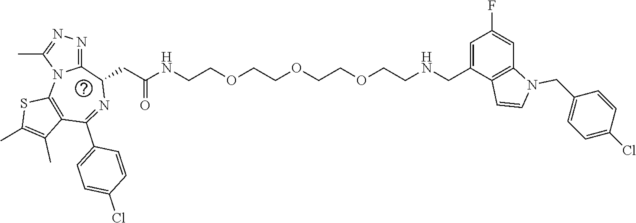 [SMILES: Cc1sc2c(c1C)C(c1ccc(Cl)cc1)=N[C@@H](CC(=O)NCCOCCOCCOCCNCc1cc(F)cc3c1ccn3Cc1ccc(Cl)cc1)c1nnc(C)n1-2] | ¹H NMR (400 MHz, DMSO-d₆) δ (ppm): 8.27 (t, J = 5.6 Hz, 1H), 8.15 (s, 1H; HCOOH), 7.49-7.46 (m, 3H), 7.41 (d, J = 8.8 Hz, 2H), 7.36 (d, J = 8.4 Hz, 2H), 7.25- 7.20 (m, 3H), 6.93 (dd, J = 10.8 Hz, 2.0 Hz, 1H), 6.61 (d, J = 3.2 Hz, 1H), 5.38 (s, 2H), 4.52- |
| | | | 4.48 (m, 1H), 4.03 |
| | | | (s, 2H), 3.55-3.49 |
| | | | (m, 10H), 3.44 (t, |
| | | | J = 6.0 Hz, 2H), |
| | | | 3.28-3.17 (m, 4H), |
| | | | 2.77 (t, J = 5.6 Hz, |
| | | | 2H), 2.59 (s, 3H), |
| | | | 2.40 (s, 3H), 1.61 |
| | | | (s, 3H). |
| | | | MS (ESI) m/z |
| | | | 846.1 |
| | | | [C₄₃H₄₆Cl₂FN₇O₄S + |
| | | | H]⁺. |
| 105 | K | 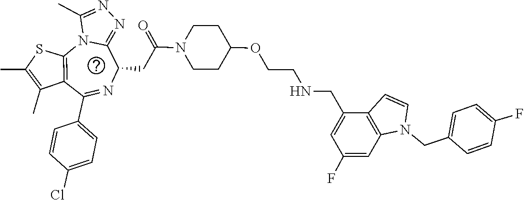 [SMILES: Cc1sc2c(c1C)C(c1ccc(Cl)cc1)=N[C@@H](CC(=O)N1CCC(OCCNCc3cc(F)cc4c3ccn4Cc3ccc(F)cc3)CC1)c1nnc(C)n1-2] | ¹H NMR (400 MHz, DMSO-d₆) δ (ppm): 8.18 (s, 1H; HCOOH), 7.51- 7.42 (m, 5H), 7.29-7.24 (m, 3H), 7.14 (t, J = 8.8 Hz, 2H), 6.94 (dd, J = 10.4 Hz, 2.0 Hz, 1H), 6.62 (d, J = 2.8 Hz, 1H), 5.37 (s, 2H), 4.57 (t, J = 6.8 Hz, 1H), 4.04 |
| | | | (s, 2H), 3.86-3.82 |
| | | | (m, 2H), 3.64-3.55 |
| | | | (m, 3H), 3.40-3.37 |
| | | | (m, 3H), 3.14-3.08 |
| | | | (m, 1H), 2.77 (t, J = |
| | | | 5.2 Hz, 2H), 2.59 |
| | | | (s, 3H), 2.41 (s, |
| | | | 3H), 1.93-1.89 (m, |
| | | | 1H), 1.76-1.73 (m, |
| | | | 1H), 1.63 (s, 3H), |
| | | | 1.59-1.51 (m, 1H), |
| | | | 1.36-1.31 (m, 1H). |
| | | | MS (ESI) m/z |
| | | | 782.1 |
| | | | [C₄₂H₄₂ClF₂N₇O₂S + |
| | | | H]⁺. |
| 106 | K | 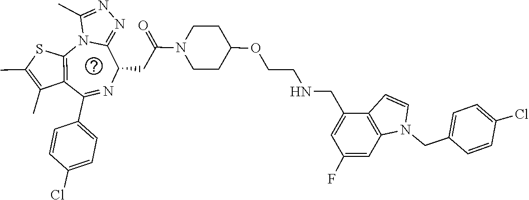 [SMILES: Cc1sc2c(c1C)C(c1ccc(Cl)cc1)=N[C@@H](CC(=O)N1CCC(OCCNCc3cc(F)cc4c3ccn4Cc3ccc(Cl)cc3)CC1)c1nnc(C)n1-2] | ¹H NMR (400 MHz, DMSO-d₆) δ (ppm): 8.16 (s, 1H; HCOOH), 7.52- 7.47 (m, 3H), 7.47 (d, J = 8.8 Hz, 2H), 7.37 (d, J = 8.4 Hz, 2H), 7.25-7.21 (m, 3H), 6.95 (dd, J = 10.4 Hz, 1.6 Hz, 1H), 6.63 (br. s, 1H), 5.39 (s, 2H), 4.57 (t, J = 6.4 Hz, |
| | | | 1H), 4.05 (s, 2H), |
| | | | 3.86-3.82 (m, 2H), |
| | | | 3.65-3.55 (m, 3H), |
| | | | 3.38-3.34 (m, 3H), |
| | | | 3.14-3.09 (m, 1H), |
| | | | 2.78 (t, J = 5.2 Hz, |
| | | | 2H), 2.59 (s, 3H), |
| | | | 2.41 (s, 3H), 1.92- |
| | | | 1.89 (m, 1H), |
| | | | 1.76-1.74 (m, 1H), |
| | | | 1.63 (s, 3H), 1.56- |
| | | | 1.51 (m, 1H), |
| | | | 1.36-1.31 (m, 1H). |
| | | | MS (ESI) m/z |
| | | | 798.1 |
| | | | [C₄₂H₄₂Cl₂FN₇O₂S + |
| | | | H]⁺. |
| 107 | K | 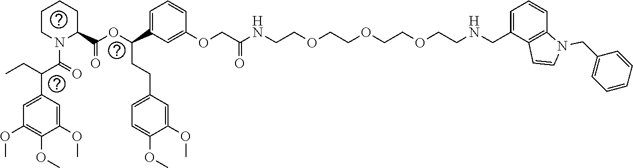 [SMILES: CCC(C(=O)N1CCCC[C@H]1C(=O)O[C@H](CCc1ccc(OC)c(OC)c1)c1cccc(OCC(=O)NCCOCCOCCOCCNCc2cccc3c2ccn3Cc2ccccc2)c1)c1cc(OC)c(OC)c(OC)c1] | ¹H NMR (400 MHz, DMSO-d₆) δ (ppm): 8.05 (t, J = 5.6 Hz, 1H), 7.54 (d, J = 2.8 Hz, 1H), 7.45 (br., 1H), 7.31-7.27 (m, 2H), 7.24-7.22 (m, 1H), 7.20-7.15 (m, 3H), 7.12-7.10 (m, 2H), 6.97-6.89 (m, 1H), |
| | | | 6.84-6.81 (m, 3H), |
| | | | 6.75-6.69 (m, 2H), |
| | | | 6.64-6.58 (m, 2H), |
| | | | 6.52 (s, 2H), 5.54- |
| | | | 5.51 (m, 1H), 5.43 |
| | | | (s, 2H), 5.29-5.27 |
| | | | (m, 1H), 4.48-4.46 |
| | | | (m, 2H), 4.25 (br. |
| | | | s, 2H), 4.04-3.92 |
| | | | (m, 1H), 3.87-3.82 |
| | | | (m, 1H), 3.74-3.32 |
| | | | (m, 28H), 3.29- |
| | | | 3.26 (m, 2H), 2.99 |
| | | | (br. s, 2H), 2.63- |
| | | | 2.60 (m, 1H), |
| | | | 2.15-2.13 (m, 1H), |
| | | | 1.94-1.89 (m, 2H), |
| | | | 1.68-1.55 (m, 4H), |
| | | | 1.23-1.17 (m, |
| | | | 1H), 0.80 (t, J = |
| | | | 7.6 Hz, 3H). |
| | | | MS (ESI) m/z |
| | | | 1087.8 |
| | | | [C₆₂H₇₈N₄O₁₃ + |
| | | | H]⁺. |
| 177 | K | 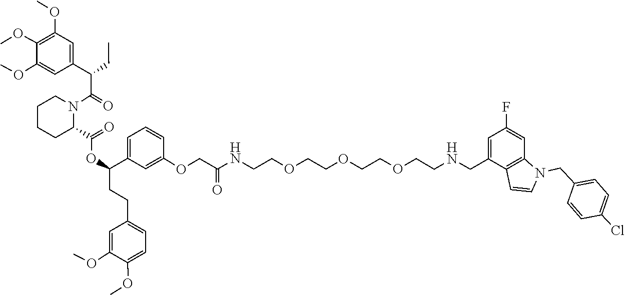 [SMILES: CC[C@H](C(=O)N1CCCC[C@H]1C(=O)O[C@H](CCc1ccc(OC)c(OC)c1)c1cccc(OCC(=O)NCCOCCOCCOCCNCc2cc(F)cc3c2ccn3Cc2ccc(Cl)cc2)c1)c1cc(OC)c(OC)c(OC)c1] | ¹H NMR (400 MHz, DMSO-d₆) δ (ppm): 8.05 (t, J = 5.6 Hz, 1H), 7.46 (d, J = 3.2 Hz, 1H), 7.37-7.35 (m, 2H), 7.21-7.19 (m, 2H), 7.17-7.14 (m, 1H), 6.97-6.81 (m, 4H), 6.75-6.71 (m, 1H), 6.69-6.62 (m, 2H), 6.58-6.57 (m, 2H), 6.53 (s, 2H), 5.54- 5.51 (m, 1H), 5.37 (s, 2H), 5.28-5.27 (m, 1H), 4.48-4.46 (m, 2H), 4.04-3.96 (m, 1H), 3.94 (s, |
| | | | 2H), 3.86-3.82 (m, |
| | | | 1H), 3.74-3.40 (m, |
| | | | 28H), 3.28-3.26 |
| | | | (m, 2H), 2.69-2.61 |
| | | | (m, 4H), 2.41-2.33 |
| | | | (m, 2H), 2.15-2.11 |
| | | | (m, 1H), 1.94-1.89 |
| | | | (m, 2H), 1.68-1.55 |
| | | | (m, 4H), 1.49-1.41 |
| | | | (m, 1H) 1.19-1.12 |
| | | | (m, 1H), 0.80 (t, |
| | | | J = 7.6 Hz, 3H). |
| | | | MS (ESI) m/z |
| | | | 1139.6 |
| | | | [C₆₂H₇₆ClFN₄O₁₃ + |
| | | | H]⁺. |
| 184 | K | 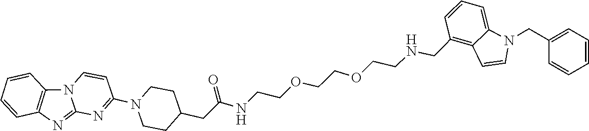 [SMILES: O=C(CC1CCN(c2ccn3c(n2)nc2ccccc23)CC1)NCCOCCOCCNCc1cccc2c1ccn2Cc1ccccc1] | ¹H NMR (400 MHz, DMSO-d₆) δ (ppm): 8.92 (d, J = 8.0 Hz, 1H), 8.21 (s, 1H; HCOOH), 7.93 (d, J = 8.0 Hz, 1H), 7.87 (t, J = 5.6 Hz, 1H), 7.49- 7.47 (m, 2H), 7.32 |
| | | | (d, J = 7.6 Hz, 1H), |
| | | | 7.30-7.26 (m, 3H), |
| | | | 7.23-7.22 (m, 1H), |
| | | | 7.18-7.12 (m, 3H), |
| | | | 7.06-7.00 (m, 2H), |
| | | | 6.86 (d, J = 7.6 Hz, |
| | | | 1H), 6.60 (d, J = |
| | | | 2.8 Hz, 1H), 5.40 |
| | | | (s, 2H), 4.52 (br. s, |
| | | | 2H), 4.03 (s, 2H), |
| | | | 3.54-3.38 (m, 8H), |
| | | | 3.19 (q, J = 5.6 Hz, |
| | | | 2H), 3.03-2.97 (m, |
| | | | 2H), 2.78 (t, J = |
| | | | 5.6 Hz, 2H), 2.02 |
| | | | (s, 3H), 1.75-1.72 |
| | | | (m, 2H), 1.17-1.12 |
| | | | (m, 2H). |
| | | | MS (ESI) m/z |
| | | | 658.1 |
| | | | [C₃₉H₄₅N₇O₃ − H]⁻. |
| 185 | K | 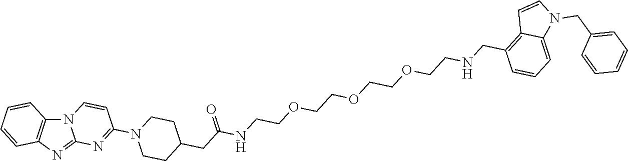 [SMILES: O=C(CC1CCN(c2ccn3c(n2)nc2ccccc23)CC1)NCCOCCOCCOCCNCc1cccc2c1ccn2Cc1ccccc1] | ¹H NMR (400 MHz, DMSO-d₆) δ (ppm): 8.93 (d, J = 8.0 Hz, 1H), 7.93 (d, J = 7.6 Hz, 1H), 7.88 (t, J = 5.6 Hz, 1H), 7.54 (d, J = 3.2 Hz, 1H), 7.48 (d, J = 8.0 Hz, 1H), 7.42-7.40 (m, 1H), |
| | | | 7.30-7.27 (m, 3H), |
| | | | 7.26-7.13 (m, 4H), |
| | | | 7.09-7.07 (m, 2H), |
| | | | 6.86 (d, J = 8.0 Hz, |
| | | | 1H), 6.66 (d, J = |
| | | | 3.2 Hz, 1H), 5.42 |
| | | | (s, 2H), 4.53 (br. s, |
| | | | 2H), 4.19 (s, 2H), |
| | | | 3.60 (t, J = 5.6 Hz, |
| | | | 2H), 3.53-3.47 (m, |
| | | | 8H), 3.40-3.37 (m, |
| | | | 2H), 3.21-3.17 (m, |
| | | | 2H), 3.04-2.94 (m, |
| | | | 4H), 1.91 (br. s, |
| | | | 3H), 1.75-1.72 (m, |
| | | | 2H), 1.18-1.10 (m, |
| | | | 2H). |
| | | | MS (ESI) m/z |
| | | | 704.6 |
| | | | [C₄₁H₄₉N₇O₄ + |
| | | | H]⁺. |
| 186 | K | 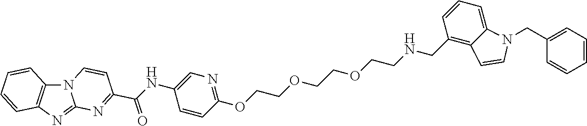 [SMILES: O=C(Nc1ccc(OCCOCCOCCNCc2cccc3c2ccn3Cc2ccccc2)nc1)c1ccn2c(n1)nc1ccccc12] | ¹H NMR (400 MHz, DMSO-d₆) δ (ppm): 11.07 (s, 1H), 9.77 (d, J = 7.2 Hz, 1H), 8.69 (d, J = 2.4 Hz, 1H), 8.43 (d, J = 8.4 Hz, 1H), 8.21 (dd, J = |
| | | | 8.8 Hz, 2.4 Hz, |
| | | | 1H), 7.96 (d, J = |
| | | | 8.4 Hz, 1H), 7.77 |
| | | | (d, J = 7.2 Hz, 1H), |
| | | | 7.64 (t, J = 7.6 Hz, |
| | | | 1H), 7.53 (t, J = |
| | | | 7.6 Hz, 1H), 7.48 |
| | | | (d, J = 3.2 Hz, 1H), |
| | | | 7.34 (d, J = 7.6 Hz, |
| | | | 1H), 7.28 (t, J = |
| | | | 7.2 Hz, 2H), 7.22 |
| | | | (d, J = 6.8 Hz, 1H), |
| | | | 7.18 (d, J = 7.2 Hz, |
| | | | 2H), 7.07-7.01 (m, |
| | | | 2H), 6.87 (d, J = |
| | | | 8.8 Hz, 1H), 6.61 |
| | | | (d, J = 2.8 Hz, 1H), |
| | | | 5.40 (s, 2H), 4.36 |
| | | | (t, J = 4.8 Hz, 2H), |
| | | | 4.03 (s, 2H), 3.74 |
| | | | (t, J = 4.8 Hz, 2H), |
| | | | 3.60-3.57 (m, 2H), |
| | | | 3.54-3.52 (m, 4H), |
| | | | 2.78 (t, J = 5.6 Hz, |
| | | | 2H). |
| | | | MS (ESI) m/z |
| | | | 656.2 |
| | | | [C₃₈H₃₇N₇O₄ + |
| | | | H]⁺. |
| 188 | K | 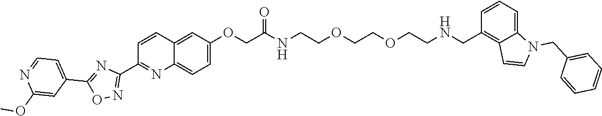 | ¹H NMR (400 MHz, DMSO-d₆) δ (ppm): 8.52-8.47 (m, 2H), 8.24-8.21 (m, 2H), 8.17 (s, 1H; HCOOH), 8.14 (d, J = 9.2 Hz, 1H), 7.74 (dd, J = |
| | | | 5.2 Hz, 1.2 Hz, |
| | | | 1H), 7.61 (dd, J = |
| | | | 9.2 Hz, 2.8 Hz, |
| | | | 1H), 7.55 (s, 1H), |
| | | | 7.48-7.46 (m, 2H), |
| | | | 7.33 (d, J = 7.6 Hz, |
| | | | 1H), 7.29-7.26 (m, |
| | | | 2H), 7.22 (d, J = |
| | | | 7.2 Hz, 1H), 7.19- |
| | | | 7.16 (m, 2H), |
| | | | 7.06-6.99 (m, 2H), |
| | | | 6.59 (d, J = 3.2 Hz, |
| | | | 1H), 5.39 (s, 2H), |
| | | | 4.68 (s, 2H), 4.03 |
| | | | (s, 2H), 3.98 (s, |
| | | | 3H), 3.52-3.46 (m, |
| | | | 8H), 3.32-3.12 (m, |
| | | | 2H), 2.77 (t, J = |
| | | | 5.6 Hz, 2H). |
| | | | MS (ESI) m/z |
| | | | 728.2 |
| | | | [C₄₁H₄₁N₇O₆ + |
| | | | H]⁺. |
| 192 | K | 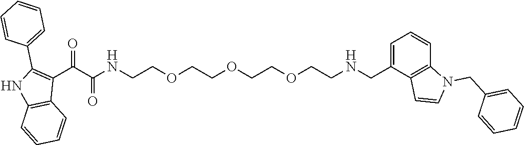 [SMILES: O=C(NCCOCCOCCOCCNCc1cccc2c1ccn2Cc1ccccc1)C(=O)c1c(-c2ccccc2)[nH]c2ccccc12] | ¹H NMR (400 MHz, DMSO-d₆) δ (ppm): 12.26 (s, 1H), 8.51 (t, J = 5.2 Hz, 1H), 8.08 (dd, J = 6.8 Hz, 1.2 Hz, 1H), 7.57- 7.54 (m, 3H), 7.49-7.42 (m, 5H), 7.31-7.20 (m, 8H), |
| | | | 7.18-7.10 (m, 2H), |
| | | | 6.67 (d, J = 2.8 Hz, |
| | | | 1H), 5.43 (s, 2H), |
| | | | 4.20 (s, 2H), 3.59 |
| | | | (t, J = 5.2 Hz, 2H), |
| | | | 3.53 (br. s, 4H), |
| | | | 3.51-3.44 (m, 4H), |
| | | | 3.21 (t, J = 6.0 Hz, |
| | | | 1H), 2.94-2.87 (m, |
| | | | 4H). |
| | | | MS (ESI) m/z |
| | | | 659.3 |
| | | | [C₄₀H₄₂N₄O₅ + |
| | | | H]⁺. |
| 193 | K | 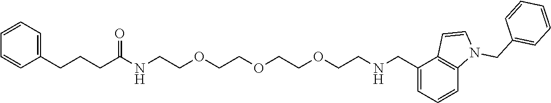 [SMILES: O=C(CCCc1ccccc1)NCCOCCOCCOCCNCc1cccc2c1ccn2Cc1ccccc1] | ¹H NMR (400 MHz, DMSO-d₆) δ (ppm): 8.17 (s, 1H; HCOOH), 7.83 (t, J = 4.8 Hz, 1H) 7.51 (d, J = 3.2 Hz, 1H), 7.36 (d, J = |
| | | | 7.6 Hz, 1H), 7.32- |
| | | | 7.23 (m, 5H), |
| | | | 7.20-7.15 (m, 5H), |
| | | | 7.09-7.02 (m, 2H), |
| | | | 6.62 (d, J = 2.8 Hz, |
| | | | 1H), 5.42 (s, 2H), |
| | | | 4.06 (s, 2H), 3.54- |
| | | | 3.46 (m, 10H), |
| | | | 3.39-3.34 (m, 2H), |
| | | | 3.20-3.15 (m, 2H), |
| | | | 2.80 (t, J = 5.6 Hz, |
| | | | 2H), 2.55-2.52 (m, |
| | | | 2H), 2.08 (t, J = |
| | | | 5.6 Hz, 2H), 1.78- |
| | | | 1.73 (m, 2H). |
| | | | MS (ESI) m/z |
| | | | 558.3 |
| | | | [C₃₄H₄₃N₃O₄ + |
| | | | H]⁺. |
| 194 | K | 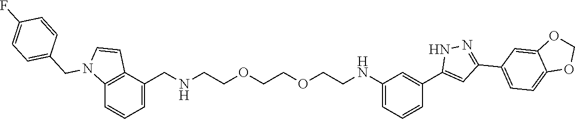 [SMILES: Fc1ccc(Cn2ccc3c(CNCCOCCOCCNc4cccc(-c5cc(-c6ccc7c(c6)OCO7)n[nH]5)c4)cccc32)cc1] | ¹H NMR (400 MHz, DMSO-d₆) δ (ppm): 13.1 (br, 1H), 7.45 (d, J = 3.2 Hz, 1H), 7.37 (s, 1H), 7.33-7.31 (m, 2H), 7.24-7.21 (m, 2H), 7.13-7.09 |
| | | | (m, 3H), 7.05-6.96 |
| | | | (m, 6H), 6.59-6.55 |
| | | | (m, 2H), 6.05 (s, |
| | | | 2H), 5.60 (br. s, |
| | | | 1H), 5.37 (s, 2H), |
| | | | 3.97 (s, 2H), 3.60- |
| | | | 3.50 (m, 6H), |
| | | | 3.39-3.35 (m, 2H), |
| | | | 3.25-3.23 (m, 2H), |
| | | | 2.72 (t, J = 5.6 Hz, |
| | | | 2H). |
| | | | MS (ESI) m/z |
| | | | 648.3 |
| | | | [C₃₈H₃₈FN₅O₄ + |
| | | | H]⁺. |
| 195 | K | 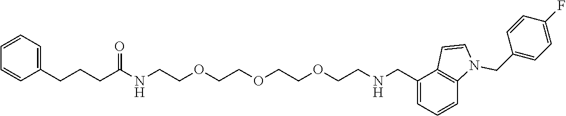 [SMILES: O=C(CCCc1ccccc1)NCCOCCOCCOCCNCc1cccc2c1ccn2Cc1ccc(F)cc1] | ¹H NMR (400 MHz, DMSO-d₆) δ (ppm): 8.26 (s, 1H; HCOOH), 7.84 (t, J = 5.2 Hz, 1H), 7.50 (d, J = 3.2 Hz, 1H), 7.37 (d, J = 7.6 Hz, 1H), 7.28- |
| | | | 7.23 (m, 4H), |
| | | | 7.17-7.03 (m, 7H), |
| | | | 6.62 (d, J = 2.8 Hz, |
| | | | 1H), 5.40 (s, 2H), |
| | | | 4.04 (s, 2H), 3.53- |
| | | | 3.45 (m, 10H), |
| | | | 3.37 (t, J = 6.0 Hz, |
| | | | 2H), 3.19-3.15 (m, |
| | | | 2H), 2.78 (t, J = |
| | | | 5.6 Hz, 2H), 2.55- |
| | | | 2.51 (m, 2H), 2.07 |
| | | | (t, J = 7.2 Hz, 2H), |
| | | | 1.80-1.75 (m, 2H). |
| | | | MS (ESI) m/z |
| | | | 576.4 |
| | | | [C₃₄H₄₂FN₃O₄ + |
| | | | H]⁺. |
| 196 | K | 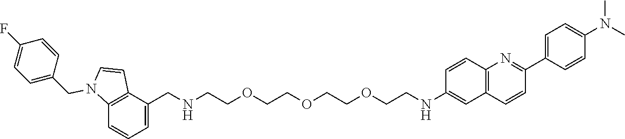 [SMILES: CN(C)c1ccc(-c2ccc3cc(NCCOCCOCCOCCNCc4cccc5c4ccn5Cc4ccc(F)cc4)ccc3n2)cc1] | ¹H NMR (400 MHz, DMSO-d₆) δ (ppm): 8.20 (s, 1H; HCOOH), 8.02 (d, J = 9.2 Hz, 2H), 7.96 (d, J = 8.4 Hz, 1H), 7.80 (d, J = 8.8 Hz, 1H), 7.68 (d, J = 9.2 Hz, 1H), |
| | | | 7.46 (d, J = 3.2 Hz, |
| | | | 1H), 7.32 (d, J = |
| | | | 8.0 Hz, 1H), 7.24- |
| | | | 7.17 (m, 3H), 7.11 |
| | | | (t, J = 9.2 Hz, 2H), |
| | | | 7.06-6.99 (m, 2H), |
| | | | 6.81 (d, J = 9.2 Hz, |
| | | | 2H), 6.70 (d, J = |
| | | | 2.4 Hz, 1H), 6.59 |
| | | | (d, J = 2.8 Hz, 1H), |
| | | | 6.03 (t, 1H), 5.37 |
| | | | (s, 2H), 3.98 (s, |
| | | | 2H), 3.62 (t, J = |
| | | | 5.6 Hz, 2H), 3.54 |
| | | | (s, 4H), 3.51-3.48 |
| | | | (m, 6H), 3.33 (m, |
| | | | 2H), 2.97 (s, 6H), |
| | | | 2.73 (t, J = 5.6 Hz, |
| | | | 2H). |
| | | | MS (ESI) m/z |
| | | | 676.3 |
| | | | [C₄₁H₄₆FN₅O₃ + |
| | | | H]⁺. |
| 198 | K | 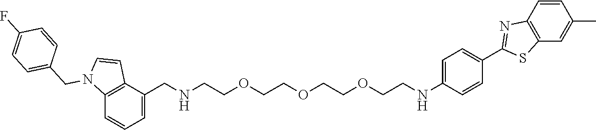 [SMILES: Cc1ccc2nc(-c3ccc(NCCOCCOCCOCCNCc4cccc5c4ccn5Cc4ccc(F)cc4)cc3)sc2c1] | ¹H NMR (400 MHz, DMSO-d₆) δ (ppm): 7.80-7.75 (m, 4H), 7.47 (d, J = 2.8 Hz, 1H), 7.32 (d, J = 7.6 Hz, 1H), 7.28-7.21 (m, 3H), 7.11 (t, J = 8.8 Hz, 2H), 7.06-7.00 (m, |
| | | | 2H), 6.69 (d, J = |
| | | | 8.8 Hz, 2H), 6.58 |
| | | | (d, J = 2.4 Hz, 1H), |
| | | | 6.40 (t, J = 5.6 Hz, |
| | | | 1H), 5.38 (s, 2H), |
| | | | 3.98 (s, 2H), 3.57- |
| | | | 3.48 (m, 13H), |
| | | | 3.27-3.23 (m, 2H), |
| | | | 2.72 (t, J = 5.6 Hz, |
| | | | 2H), 2.42 (s, 3H). |
| | | | MS (ESI) m/z |
| | | | 653.4 |
| | | | [C₃₈H₄₁FN₄O₃S + |
| | | | H]⁺. |
| 200 | K | 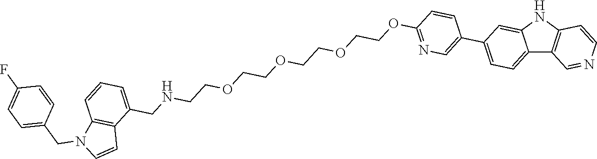 [SMILES: Fc1ccc(Cn2ccc3c(CNCCOCCOCCOCCOc4ccc(-c5ccc6c(c5)[nH]c5ccncc56)cn4)cccc32)cc1] | ¹H NMR (400 MHz, DMSO-d₆) δ (ppm): 11.83 (br. s, 1H), 9.36 (s, 1H), 8.55 (d, J = 2.4 Hz, 1H), 8.43 (d, J = 5.6 Hz, 1H), 8.30 (d, J = 8.4 Hz, 1H), 8.23 (s, 1H; HCOOH), 8.10 |
| | | | (dd, J = 8.8 Hz, 2.4 |
| | | | Hz, 1H), 7.77 (s, |
| | | | 1H), 7.55 (dd, J = |
| | | | 8.0 Hz, 1.2 Hz, |
| | | | 1H), 7.51 (d, J = |
| | | | 3.2 Hz, 1H), 7.48 |
| | | | (d, J = 5.6 Hz, 1H), |
| | | | 7.38 (dd, J = 6.8 |
| | | | Hz, 1.6 Hz, 1H), |
| | | | 7.25-7.21 (m, 2H), |
| | | | 7.13-7.05 (m, 4H), |
| | | | 6.94 (d, J = 8.8 Hz, |
| | | | 1H), 6.64 (d, J = |
| | | | 2.8 Hz, 1H), 5.39 |
| | | | (s, 2H), 4.42 (t, J = |
| | | | 4.8 Hz, 2H), 4.11 |
| | | | (s, 2H), 3.75 (d, J = |
| | | | 4.8 Hz, 2H), 3.57- |
| | | | 3.52 (m, 10H), |
| | | | 2.85 (t, J = 5.2 Hz, |
| | | | 2H). |
| | | | MS (ESI) m/z |
| | | | 674.2 |
| | | | [C₄₀H₄₀FN₅O₄ + |
| | | | H]⁺. |
| 203 | K | 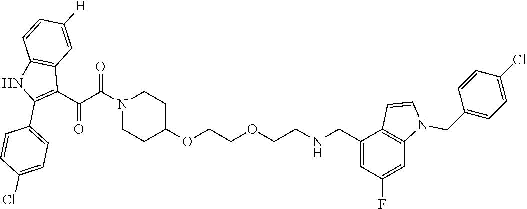 [SMILES: O=C(C(=O)N1CCC(OCCOCCNCc2cc(F)cc3c2ccn3Cc2ccc(Cl)cc2)CC1)c1c(-c2ccc(Cl)cc2)[nH]c2ccccc12] | ¹H NMR (400 MHz, DMSO-d₆) δ (ppm): 12.60 (br. s, 1H), 8.08 (d, J = 7.6 Hz, 1H), 7.61- 7.55 (m, 4H), 7.50 (d, J = 6.4 Hz, 1H), 7.44 (d, J = 3.2 Hz, 1H), 7.36-7.32 (m, 2H), 7.31-7.23 (m, 2H), 7.19 (d, J = 8.4 Hz, 2H), 7.17-7.13 (m, 1H), |
| | | | 6.90-6.87 (m, 1H), |
| | | | 6.57 (d, J = 3.2 Hz, |
| | | | 1H), 5.35 (s, 2H), |
| | | | 3.94 (s, 2H), 3.51- |
| | | | 3.44 (m, 7H), |
| | | | 3.39-3.29 (m, 2H), |
| | | | 3.01-2.96 (m, 1H), |
| | | | 2.89-2.80 (m, 1H), |
| | | | 2.70-2.67 (m, 2H), |
| | | | 1.67-1.59 (m, 2H), |
| | | | 1.30-1.17 (m, 2H). |
| | | | MS (ESI) m/z |
| | | | 741.3 |
| | | | [C₄₁H₃₉Cl₂FN₄O₄ + |
| | | | H]⁺. |
| 204 | K | 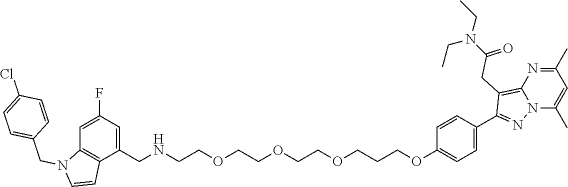 [SMILES: CCN(CC)C(=O)Cc1c(-c2ccc(OCCCOCCOCCOCCNCc3cc(F)cc4c3ccn4Cc3ccc(Cl)cc3)cc2)nn2c(C)cc(C)nc12] | ¹H NMR (400 MHz, DMSO-d₆) δ (ppm): 7.68 (d, J = 8.8 Hz, 2H), 7.45 (d, J = 3.6 Hz 1H), 7.37-7.35 (m, 2H), 7.21-7.16 (m, 3H), 7.01 (d, J = 8.8 Hz, 2H), 6.90-6.86 (m, 1H), 6.83 (s, 1H), 6.57 (d, J = 2.4 Hz, 1H), 5.36 (s, 2H), |
| | | | 4.28 (t, 2H), 3.93 |
| | | | (s, 2H), 3.82 (s, |
| | | | 2H), 3.55-3.44 (m, |
| | | | 14H), 3.29-3.25 |
| | | | (m, 2H), 2.68-2.66 |
| | | | (m, 5H), 2.49 (s, |
| | | | 3H), 1.95-1.91 (m, |
| | | | 2H), 1.17 (t, J = |
| | | | 7.2 Hz, 3H), 1.01 |
| | | | (t, J = 7.2 Hz, 3H). |
| | | | MS (ESI) m/z |
| | | | 813.3 |
| | | | [C₄₅H₅₄ClFN₆O₅ + |
| | | | H]⁺. |

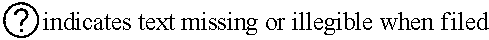

### Example 24: Surface Plasmon Resonance (SPR) Analysis

### Protein Preparation

Recombinant p62 protein was buffer exchanged into Biacore SPR running buffer (Cytiva 100671) using the Slide-A-Lyzer Mini Dialysis system 7K MWCO (Thermo Fisher Scientific, Cat no. 69560). The final protein concentration was determined using the Nanodrop One spectrophotometer (Thermo Fisher Scientific, cat no. ND-ONE-W), with calibration for the dialysis buffer.

### Protein Immobilisation

The prepared protein was diluted in sodium acetate buffer (NaAc, pH 5.5, Cytiva) to concentrations ranging from approximately 0.25-40 μg/mL. Post-immobilization, the protein required approximately 16 hours of equilibration in running buffer supplemented with 1% DMSO at 15° C. to ensure adequate surface equilibration and protein reorganization.

### SPR Running Conditions

All SPR experiments were conducted at a constant temperature of 15° C. The SPR chip surface was activated using EDC/NHS for a contact time of 6-10 min at a flow rate of 10 μL/min. This was followed by protein immobilization for 2100 seconds and ethanolamine capping for 420 seconds, both at the same flow rate. Immediately after immobilization, the NH2-REEE peptide was analyzed in Single Cycle Kinetics mode (SCK) to verify surface activity, ensuring it binds the sample with the published affinity (˜20 μM by SPR).

### Compound Binding Experiments

For general compound binding studies, experiments were performed at 15° C. using a running buffer composed of 10 mM HEPES, 150 mM NaCl, 1 mM DTT, 0.05% Tween 20, and 1% DMSO at pH 7.4. The test compound was initially prepared at a 20 mM concentration in DMSO and subsequently titrated in 2-fold dilutions. For each assay, 2 μL of the compound was mixed with 198 μL of the running buffer, and the mixture was thoroughly pipetted approximately 15 times for homogenization.

A series of blank injections (0 μM concentration) were included for double referencing, with at least 8 or 16 per plate depending on the format. Typical injection parameters were 120 seconds for association and 180 seconds for dissociation at a flow rate of 50 L/min. A four-point solvent correction was prepared with DMSO concentrations ranging from 2% to 0.5% in the buffer. Post each compound injection, the system's needles were washed with 50% DMSO.

### Data Acquisition and Analysis

Analysis of the SPR data was conducted using the Cytiva Evaluation software. The binding levels at the late time-point of each interaction were corrected for reference, then normalized to percentage bound values to account for variations in immobilization levels, protein molecular weight, and compound molecular weight.

### Example 25: Western Blot Protocol

HeLa cells were seeded in 6-well plates (700,000 cells) and allowed to adhere overnight. Media was replaced one hour before administering either vehicle (DMSO) or test compounds. After 24 hours of treatment, the media was discarded, and cells were rinsed once with ice-cold 1×PBS before detached from plates with ice-cold RIPA Lysis and Extraction Buffer (Thermo Fisher Scientific) which was supplemented with protease and phosphatase inhibitors. The resulting cell suspensions were transferred to pre-chilled microcentrifuge tubes, incubated on ice for 20 min with intermittent mixing and then centrifuged at 12,000 rpm for 20 min at 4° C. The supernatants were then aspirated slowly and transferred into new tubes kept on ice. The protein concentration in the samples was determined using BCA kit (Thermo Fisher Scientific) as per manufacturer's instructions.

For the p62 oligomerization and LC3 lipidation assays, cell lysates were prepared using RIPA buffer supplemented with 2% SDS and benzonase (Sigma). The lysates were then combined with 4× Laemmli protein sample buffer (Bio-Rad), heated at 70° C. for 10 min and subjected to a brief centrifugation of 10-15 seconds.

For the BRD4 degradation assay, cell lysates were prepared with RIPA buffer containing 0.25% SDS. These were mixed with 4× Laemmli protein sample buffer (Bio-Rad) supplemented with beta-mercaptoethaol (355 mM). The lysates were then heated at 95° C. for 5 min, and briefly centrifuged for 10-15 seconds.

For the FKBP12(F36V) degradation assay, cell lysates were prepared with a Triton-X-100 buffer supplemented with 2% SDS. The lysates were mixed with 4× Laemmli protein sample buffer (Bio-Rad) supplemented with beta-mercaptoethaol (355 mM). The lysates were then heated at 95° C. for 5 min, and briefly centrifuged for 10-15 seconds.

For p62 oligomerization and LC3 lipidation assays, lysates containing 30 μg of protein and for BRD4 and FKBP12(F36V) degradation assays, lysates containing 20 μg of protein, were loaded slowly into the wells of 4-20% gradient Tris-glycine gels (Bio-Rad). The gels were run at 100V until completion and transferred onto PVDF membranes. Membranes were blocked using 5% milk in TBS-Tween (0.1%) buffer at room temperature for 1 hour. The membranes were incubated with primary antibody in 5% milk-TBST with shaking, overnight at 4° C. The primary antibodies used were: p62 (Novus Biologicals 2C11, H00008878-MO1), LC3A/B (Cell Signalling Technology D3U4C, 12741S), FKBP12 (Abcam ab24373), BRD4 (Cell Signalling Technology E2A7X, 13440S), GAPDH (Cell Signalling Technology D4C6R, 97166S), and β-Actin (Proteintech 66009-1-Ig). Following primary antibody incubation, membranes were washed thrice with TBST 10 min before and after incubation with secondary-HRP conjugated antibody in 5% milk-TBST for 1 hour at room temperature. The secondary antibodies used were: Anti-Mouse IgG HRP Conjugate (Promega W4021B) and Anti-Rabbit IgG HRP Conjugate (Promega W4011). For the p62 oligomerization assay, the total protein levels were assessed using Ponceau staining (Thermo Fisher Scientific) of PVDF membrane as per recommendation by manufacturer's guidelines.

Imaging was acquired using SuperSignal™ West Pico PLUS Chemiluminescent Substrate (Thermo Fisher Scientific) with iBright Invitrogen FL100 imaging system. Band signal intensity, reflective of protein concentrations, were quantified using Invitrogen™ iBright™ Analysis and Image J software.

### Example 26: Immunocytochemistry and Imaging Protocol

HeLa cells were seeded in coated 96-well plates (10,000 cells) and allowed to adhere overnight. After 24 hours of treatment with either vehicle (DMSO) or test compounds, the media was discarded, and cells were rinsed with ice-cold 1×PBS before fixation by 4% formaldehyde or paraformaldehyde for 20 min. Cells were then washed with 1×PBS and permeabilized with 0.1% Triton X-100 in PBS for 20 min and blocked with Blocking Buffer in PBS for 1 hour. The cells were incubated thereafter for 1 hour with anti-p62 primary antibody in Blocking Buffer, followed by incubation with secondary antibody for 1 hour. DAPI dye was used to visualize nuclei of fixed cells. The blocking buffer used were 5% Fetal Bovine Serum (FBS, ThermoFIsher Scientific, 10270106) and Li-cor Intercept (PBS) Blocking Buffer (Li-cor, 927-70001). The primary antibody used was mouse anti-p62 primary antibody (Novus Biologicals, H00008878-M01). The secondary antibodies used were goat anti-mouse Alexa Fluor Plus 488 secondary antibody (Invitrogen, GOXMS) and goat anti-mouse Alexa Fluor 488 secondary antibody (Invitrogen, A-11029). The DAPI dyes used were DAPI dye (Invitrogen, 62248) and DAPI dye (Invitrogen, D1306).

For the EVOS M7000 System, cells were imaged on the EVOS M7000 microscope using a 20× air objective (Nikon, N.A 0.45). DAPI and p62 channels were illuminated with LED light cubes using integrated hard-coated filter sets for DAPI (357/447 nm) and GFP (470/525 nm) respectively. Images were analysed for p62 puncta count on the Celeste 6.0 software.

For the Operetta CLS System, cells were imaged on the Operetta CLS using a 63× water objective on the DAPI and Alexa 488 channels. Images were analysed for p62 puncta count on the Harmony 5.1 software.

### Example 27: Studies on Compound Binding to p62

Compound binding studies were carried out according to experimental procedures above and SPR % occupancy was determined and summarized in Table 7.

TABLE 7
SPR binding data (% occupancy at 100 μM)

| Cpd No. | SPR % occupancy |
|---|---|
| 1 | ++ |
| 2 | + |
| 3 | ++ |
| 4 | + |
| 5 | ++ |
| 6 | ++ |
| 7 | + |
| 8 | + |
| 9 | +++ |
| 10 | +++ |
| 11 | +++ |
| 12 | ++ |
| 13 | ++ |
| 14 | + |
| 15 | ++ |
| 16 | +++ |
| 17 | ++ |
| 21 | ++ |
| 22 | + |
| 24 | ++ |
| 25 | + |
| 26 | ++ |
| 27 | +++ |
| 29 | + |
| 31 | +++ |
| 34 | +++ |
| 35 | +++ |
| 36 | ++ |
| 37 | ++ |
| 38 | ++ |
| 40 | + |
| 42 | + |
| 43 | + |
| 44 | +++ |
| 45 | ++ |
| 46 | ++ |
| 47 | + |
| 48 | ++ |
| 49 | ++ |
| 50 | + |
| 51 | + |
| 52 | + |
| 53 | + |
| 54 | + |
| 84 | + |
| 85 | + |

Legend:
+: % occupancy <30
++: % occupancy 30-70
+++: % occupancy >70

### Example 28: Oligomerization Activity of p62 Protein

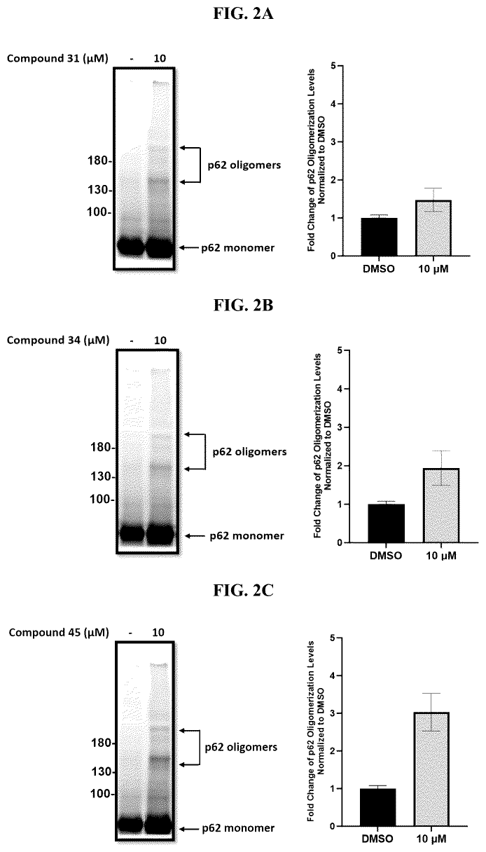

HeLa cells were treated with compounds for 24 hours to evaluate the effect of these compounds on p62 oligomerisation activity. The resulting changes in levels of p62 oligomers were assessed by Western blotting, as described in experimental procedures. The fold change of p62 oligomer levels induced by compound treatment relative to vehicle control (DMSO) was determined and summarized in Table 8. Representative Western blot images are shown in FIGS. 2A-C. Specifically, FIGS. 2A-C show Western blot images and their corresponding quantification showing the levels of high molecular weight (100-200 kDa) p62 oligomers by 24 hours compound treatments at listed concentrations, relative to vehicle control (DMSO) in HeLa cells. Protein loading consistencies was assessed by Ponceau staining (not shown).

TABLE 8
Compound effect on p62 oligomerization

| Cpd No. | p62 oligomerization |
|---|---|
| 1 | ++ |
| 6 | ++ |
| 9 | ++ |
| 10 | ++ |
| 11 | ++ |
| 13 | ++ |
| 21 | + |
| 26 | ++ |
| 27 | ++ |
| 31 | ++ |
| 34 | ++ |
| 35 | ++ |
| 37 | ++ |
| 44 | ++ |
| 45 | ++ |
| 48 | ++ |
| 49 | + |

Legend:
++: >2-fold change relative to DMSO control
+: <2-fold change relative to DMSO control

### Example 29: Lipidation Activity of LC3 Protein

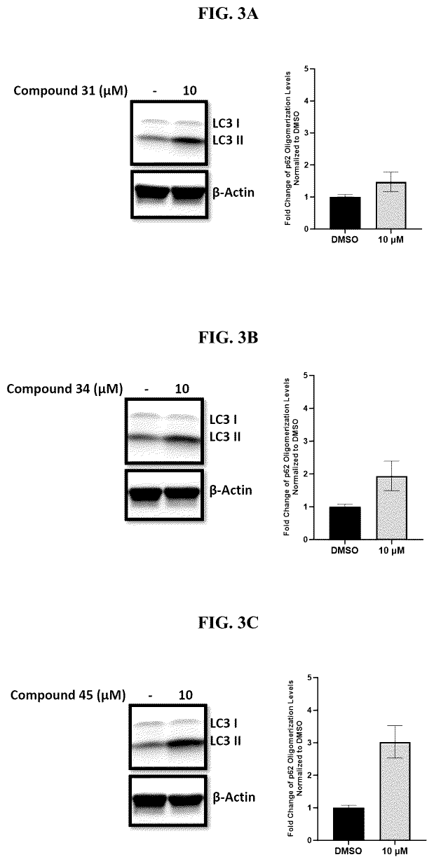

HeLa cells were treated with compounds for 24 hours to evaluate the effect of these compounds on LC3-II/LC3-I levels, a measure of autophagy activation. The changes in levels of LC3-I, LC3-II and 3-Actin (loading control) were assessed by Western blotting, as described in experimental procedures. The fold increase of LC3-II/LC3-I levels induced by compound treatment relative to vehicle control (DMSO) was determined and summarized in Table 9. Representative Western blot images are shown in FIGS. 3A-C. Specifically, FIGS. 3A-C show Western blot images and their corresponding quantification showing the lipidation levels of LC3 protein (LC3-II/LC3-I) by 24 hours compound treatments at listed concentrations, relative to vehicle control (DMSO) in HeLa cells.

TABLE 9
Compound effect on LC3 lipidation

| Cpd No. | LC3 lipidation |
|---|---|
| 1 | + |
| 6 | + |
| 9 | + |
| 10 | + |
| 11 | ++ |
| 13 | + |
| 21 | − |
| 26 | + |
| 27 | + |
| 31 | + |
| 34 | + |
| 35 | ++ |
| 37 | + |
| 44 | ++ |
| 45 | ++ |
| 48 | ++ |
| 49 | − |

Legend:
++: >2-fold change in LC3-II/LC3-I relative to DMSO control
+: <2-fold change in LC3-II/LC3-I relative to DMSO control
−: no change in LC3-II/LC3-I relative to DMSO control

### Example 30: Puncta Activity of p62 Protein

HeLa cells were treated with compounds for 24 hours to evaluate the effect of these compounds on p62 puncta activity. The resulting changes in levels of p62 puncta were assessed by imaging, either on the EVOS M7000 or Operetta CLS systems as described in experimental procedures. The fold change of p62 puncta levels induced by compound treatment relative to vehicle control (DMSO) was determined and summarized in Table 10.

TABLE 10
Compound effect on p62 puncta

| Cpd No. | p62 puncta |
|---|---|
| 1 | + |
| 3 | + |
| 5 | + |
| 7 | + |
| 39 | ++ |
| 44 | ++ |
| 45 | ++ |
| 54 | ++ |
| 77 | ++ |
| 80 | ++ |
| 81 | ++ |
| 83 | ++ |
| 86 | ++ |
| 87 | ++ |
| 88 | + |
| 89 | ++ |
| 90 | ++ |
| 93 | ++ |
| 94 | ++ |
| 108 | ++ |
| 110 | ++ |
| 111 | + |
| 112 | ++ |
| 113 | + |
| 114 | + |
| 117 | ++ |
| 118 | ++ |
| 119 | ++ |
| 120 | ++ |
| 121 | ++ |
| 122 | ++ |
| 123 | ++ |
| 125 | + |
| 126 | + |
| 127 | ++ |
| 128 | ++ |
| 129 | ++ |
| 130 | ++ |
| 133 | ++ |
| 134 | ++ |
| 135 | ++ |
| 136 | ++ |
| 137 | ++ |
| 138 | ++ |
| 139 | + |
| 140 | ++ |
| 141 | + |
| 142 | + |
| 143 | ++ |
| 144 | + |
| 145 | + |
| 146 | ++ |
| 147 | + |
| 148 | + |
| 149 | + |
| 150 | + |
| 151 | + |
| 152 | + |
| 154 | ++ |
| 155 | + |
| 156 | ++ |
| 157 | ++ |
| 158 | ++ |
| 159 | ++ |
| 160 | ++ |
| 161 | ++ |
| 162 | ++ |
| 163 | ++ |
| 164 | ++ |
| 165 | ++ |
| 166 | ++ |
| 167 | ++ |
| 168 | ++ |
| 169 | + |
| 170 | ++ |
| 171 | ++ |
| 172 | ++ |
| 173 | ++ |
| 174 | ++ |
| 175 | ++ |
| 176 | ++ |
| 178 | ++ |

Legend:
++: >2-fold change in p62 puncta levels relative to DMSO control
+: <2-fold change in p62 puncta levels relative to DMSO control

### Example 31: Degradation of BRD4

To evaluate levels of protein degradation by compounds 97-102, HeLa cells were treated with the corresponding compounds at doses up to 10 μM for 24 hours. The changes in levels of BRD4 and GAPDH (loading control) were assessed by Western blotting, as described in experimental procedures. Results are summarized in Table 11 below, measured by percentage change in BRD4 levels by compound treatment relative to vehicle (DMSO) control.

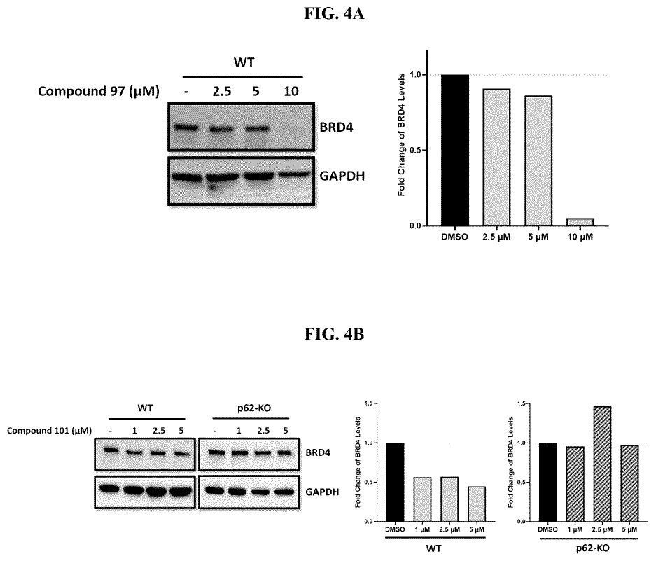

To demonstrate p62 dependency of the compounds, wild type (WT) and p62 knockout (p62-KO) HeLa cells were treated with respective compounds for 24 hours. Compound-mediated BRD4 degradation was not observed in p62-KO cells in contrast to WT HeLa cells. Representative Western blot images are shown in FIGS. 4A-B. Specifically, FIGS. 4A-B show Western blot images and their corresponding quantifications showing the degradation levels of BRD4 by 24 hours compound treatments at listed concentrations, relative to vehicle control (DMSO) in wild type (WT) and/or p62 knockout (p62-KO) HeLa cells.

TABLE 11
BRD4 degradation by compounds 97-102

| Cpd No. | Degradation |
|---|---|
| 97 | ++ |
| 98 | + |
| 99 | ++ |
| 100 | ++ |
| 101 | ++ |
| 102 | + |

### Example 32: Degradation of FKBP12(F36V)

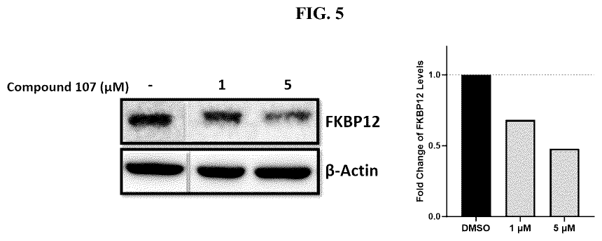

HeLa cells expressing FKBP12-F36V were treated with compound 107 for 24 hours to evaluate its ability to degrade the target protein. The changes in levels of FKBP12 and 3-Actin (loading control) were assessed by Western blotting, as described in experimental procedures. Representative Western blots images are shown in FIG. 5. Specifically, FIG. 5 shows Western blot images and their corresponding quantification showing the degradation levels of FKBP12 by 24 hours compound treatment at listed concentrations, relative to vehicle control (DMSO) in HeLa cells expressing FKBP12(F36V).

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A compound of formula (I) 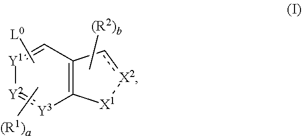
or a pharmaceutically acceptable salt, stereoisomer, or deuterated form thereof,
wherein:
L⁰ is —C₁₋₆ alkylene-NR^{A}R^{B}, —C₁₋₆ alkylene-NR^{A}—C₁₋₆ alkylene-R^{B}, —C₁₋₆ alkylene-NR^{A}C(O)NR^{A}R^{B}, —C₁₋₆ alkylene-NR^{A}C(O)—R^{B}, —C₁₋₆ alkylene-O—C₁₋₆ alkylene-NR^{A}R^{B}, —C₁₋₆ alkylene-O—C₁₋₆ alkylene-C(O)NR^{A}R^{B}, —C₁₋₆ alkylene-O—C₁₋₆ alkylene-NR^{A}C(O)—R^{B}, —C₁₋₆ alkylene-heterocyclylene-O—C₁₋₆ alkyl, —C₃₋₈ cycloalkylene-NR^{A}R^{B}, —C(O)NR^{A}R^{B}, —O—C₁₋₆ alkylene-C(O)NR^{A}R^{B}, —O—C₁₋₆ alkylene-NR^{A}R^{B}, —NR^{A}C(O)R^{B}, —NR^{A}C(O)NR^{A}R^{B}, or —NR^{A}C(O)NR^{A}—C₁₋₆ alkylene-R^{B}, wherein the alkylene is optionally substituted with —OH or halogen;
each R¹ is independently C₁₋₆ alkyl, C₃-C₈ cycloalkyl, or halogen;
each R² is independently C₁₋₆ alkyl, C₃-C₈ cycloalkyl, or halogen, or two R² form an oxo;
- - is a bond or absent;
one of X¹ or X² is N-Q-R³ and the other is CH or CH₂ as permitted by valency, provided that when X² is N-Q-R³, - - is absent;
Y¹, Y², and Y³ are each independently CH or N;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, or —C(O)O—(C₁₋₆ alkyl);
each R^{A} is independently H, C₁₋₆ alkyl, C₁₋₆ haloalkyl, C₃₋₈ cycloalkyl, C₃₋₈ hydroxycycloalkyl, aryl, or heterocycle;
each R^{B} is independently C₁₋₆ alkyl, C₁₋₆ hydroxyalkyl, C₃₋₈ hydroxycycloalkyl, C₁₋₆ alkoxy, C₁₋₆ alkylene-NH₂, or C₁₋₆ alkylene-SH;
R^{X} is H, C₁₋₆ alkyl, C₁₋₆ haloalkyl, or C₁₋₆ hydroxyalkyl;
R^{Y} is C₃₋₈ cycloalkyl, C₃₋₈ hydroxycycloalkyl, aryl, or heterocycle;
a is an integer of 0-3;
b is an integer of 0-2; and
the compound of formula (I) is not

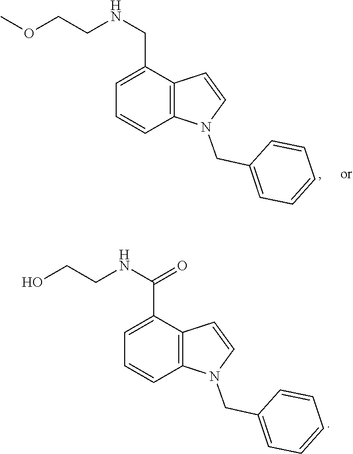 [SMILES: COCCNCc1cccc2c1ccn2Cc1ccccc1 ; O=C(NCCO)c1cccc2c1ccn2Cc1ccccc1]

2. The compound of claim 1, having a structure of formula (I-A): 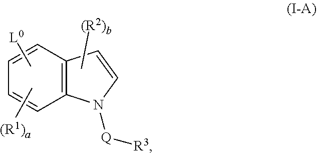
or a pharmaceutically acceptable salt, a stereoisomer, or a deuterated form thereof.

3. The compound of claim 1, having a structure of formula (I-B), (I-C), or (I-D): 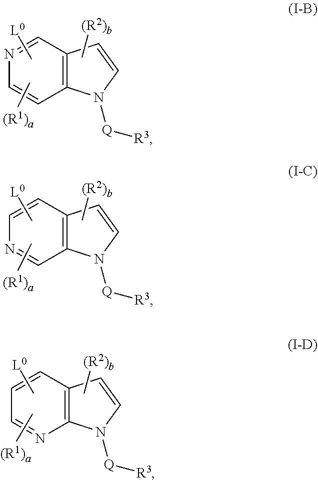
or a pharmaceutically acceptable salt, a stereoisomer, or a deuterated form thereof.

4. The compound of claim 1, having a structure of formula (I-E), (I-F), or (I-G): 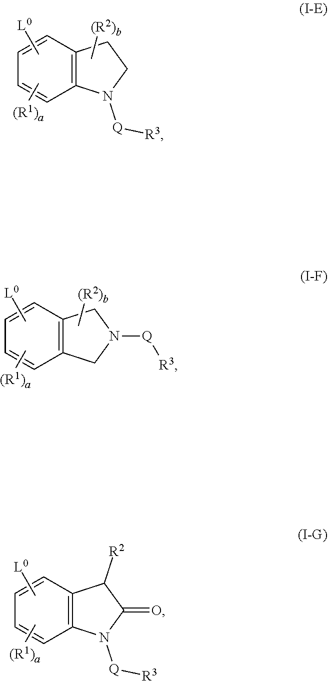
or a pharmaceutically acceptable salt, a stereoisomer, or a deuterated form thereof.

5. The compound of claim 1, having a structure of formula (I-A-1), (I-A-2), or (I-A-3): 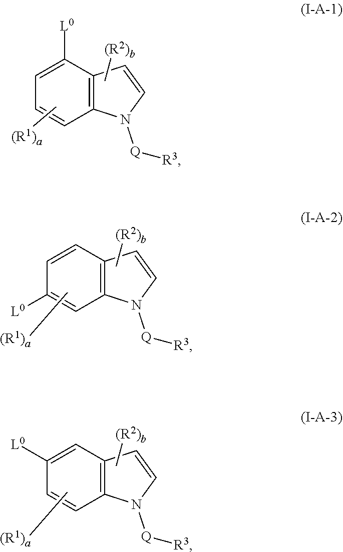
or a pharmaceutically acceptable salt, a stereoisomer, or a deuterated form thereof.

6. The compound of claim 1, having a structure of formula (I-B-1), (I-C-1), or (I-D-1): 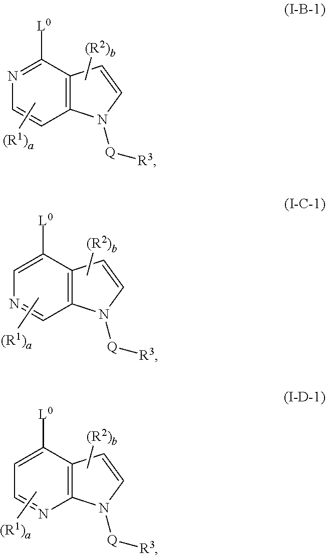
or a pharmaceutically acceptable salt, a stereoisomer, or a deuterated form thereof.

7. The compound of claim 1, having a structure of formula (I-E-1), (I-F-1), or (I-G-1): 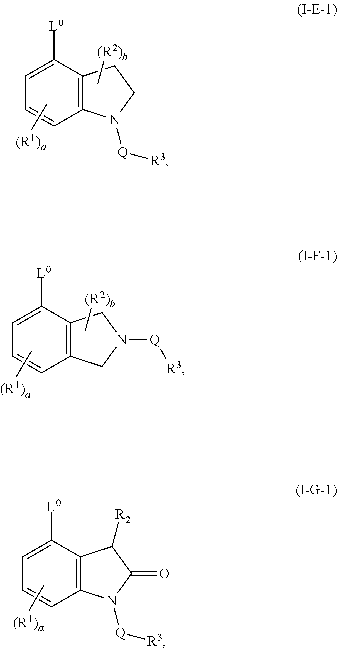
or a pharmaceutically acceptable salt, a stereoisomer, or a deuterated form thereof.

8. The compound of claim 1, or a pharmaceutically acceptable salt, stereoisomer, or deuterated form thereof, wherein L⁰ is —(C₁₋₆ alkylene)-N(H)(C₁₋₆ hydroxyalkyl), —(C₁₋₆ alkylene)-N(C₁₋₆ alkyl)(C₁₋₆ hydroxyalkyl), —(C₁₋₆ alkylene)-N(H)(C₁₋₆ alkoxy), —(C₁₋₆ alkylene)-N(C₁₋₆ alkyl)(C₁₋₆ alkoxy), —(C₁₋₆ alkylene)-NH(C₁₋₆ alkylene)(C₁₋₆ alkoxy), —C(O)N(H)(C₁₋₆ hydroxyalkyl), —(C₁₋₆ alkylene)-N(H)C(O)(C₁₋₆ alkoxy), —(C₁₋₆ alkylene)-N(C₁₋₆ alkyl)C(O)(C₁₋₆ alkoxy), —(C₁₋₆ alkylene)-N(H)C(O)N(H)(C₁₋₆ alkoxy), —(C₁₋₆ alkylene)-N(C₁₋₆ alkyl)C(O)N(C₁₋₆ alkyl)(C₁₋₆ alkoxy), —(C₃₋₈ cycloalkylene)N(H)(C₁₋₆ hydroxyalkyl), —(C₃₋₈ cycloalkylene)N(C₁₋₆ alkyl)(C₁₋₆ hydroxyalkyl), —O—(C₁₋₆ alkylene)-N(H)(C₁₋₆ hydroxyalkyl), —O—(C₁₋₆ alkylene)-N(C₁₋₆ alkyl)(C₁₋₆ hydroxyalkyl), —N(H)(C₁₋₆ hydroxyalkyl), —N(H)C(O)N(H)(C₁₋₆ hydroxyalkyl), —N(H)C(O)N(H)(C₁₋₆ alkylene)-(C₁₋₆ alkoxy), —(C₁₋₆ alkylene)-O—(C₁₋₆ alkylene)C(O)N(H)(C₁₋₆ alkyl), —(C₁₋₆ alkylene)-O—(C₁₋₆ alkylene)N(H)C(O)(C₁₋₆ alkyl), —O—(C₁₋₆ alkylene)C(O)N(H)(C₁₋₆ alkyl), —N(C₁₋₆ alkyl)(C₁₋₆ hydroxyalkyl), —N(H)C(O)(C₁₋₆ hydroxyalkyl), —N(C₁₋₆ alkyl)C(O)(C₁₋₆ hydroxyalkyl), —(C₁₋₆ alkylene)-heterocyclylene-(C₁₋₆ alkoxy), —(C₁₋₆ alkylene)-O—(C₁₋₆ alkylene)-N(H)(C₁₋₆ alkyl), or —(C₁₋₆ alkylene)-O—(C₁₋₆ alkylene)-N(C₁₋₆ alkyl)(C₁₋₆ alkyl),
wherein the alkylene or alkyl is optionally substituted with —OH or halogen.

9. The compound of claim 1, or a pharmaceutically acceptable salt, stereoisomer, or deuterated form thereof, wherein L⁰ is
—CH₂NHCH₂CH₂OH,
—CHCH₃NHCH₂CH₂OH,
—CH₂NHCH₂CH(CH₃)OH, 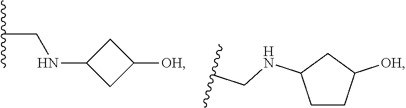 [SMILES: *CNC1CC(O)C1 ; *CNC1CCC(O)C1]
—CH₂NHCH₂CH₂OCH₃,
—CH₂NHCH₂CH₂OCH(CH₃)₂,
—CH₂OCH₂CH₂NHCH₃,
—CH₂OCH₂CH₂NHCH(CH₃)₂,
—CH₂OCH₂CH₂N(CH₃)₂,
—C(O)NHCH₂CH₂OH,
—NHC(O)CH₂CH₂OH,
—NHC(O)CH₂OH,
—CH₂NHC(O)NHCH₂CH₂OH, 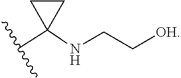 [SMILES: *C1(NCCO)CC1]
—NHC(O)NHCH₂CH₂OH,
—OCH₂CH(OH)CH₂NHCH(CH₃)₂,
—OCH₂CH₂C(O)NHCH₃,
—CH₂OCH₂CH₂C(O)NHCH₃,
—CH₂OCH₂CH₂NHC(O)CH₃,
—CH₂NHC(O)CH₂OH,
—CH₂NHC(O)CH₂CH₂OH,
—NHC(O)NHCH₂CH₂OCH₃, or

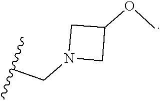 [SMILES: *CN1CC(OC)C1]

10. The compound of claim 1, or a pharmaceutically acceptable salt, stereoisomer, or deuterated form thereof, wherein L⁰ is —CH₂NHCH₂CH₂OH, —CH₂NHCH₂CH(CH₃)OH, —CH₂OCH₂CH₂NHCH₃, or —OCH₂CH(OH)CH₂NHCH(CH₃)₂.

11. The compound of claim 1, having a structure of formula (I-A-1-a), (I-A-2-a), or (I-A-3-a): 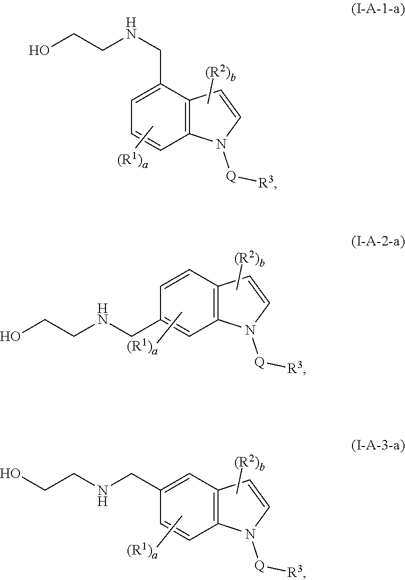
or a pharmaceutically acceptable salt, a stereoisomer, or a deuterated form thereof.

12. The compound of claim 1, having a structure of formula (I-B-1-a), (I-C-1-a), or (I-D-1-a): 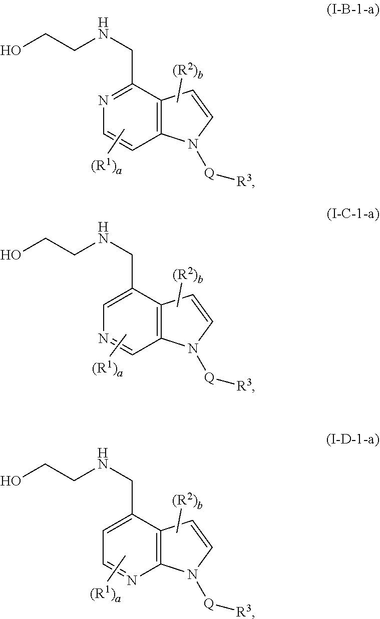
or a pharmaceutically acceptable salt, a stereoisomer, or a deuterated form thereof.

13. The compound of claim 1, having a structure of formula (I-E-1-a), (I-F-1-a), or (I-G-1-a): 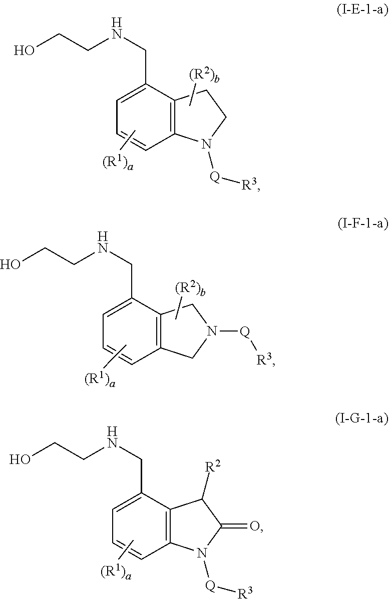
or a pharmaceutically acceptable salt, a stereoisomer, or a deuterated form thereof.

14. The compound of claim 1, wherein at least one H in L⁰ is replaced by conjugate comprising a ligand that binds to a protein, a protein aggregate, a protein complex, or a lipid.

15. A compound of formula (II) 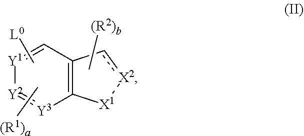
or a pharmaceutically acceptable salt, stereoisomer, or deuterated form thereof,
wherein:
L¹ is C₂-C₅₀ alkylene-R⁵, C₂-C₂₅ alkenylene-R⁵, C₂-C₂₅ alkynylene-R⁵, wherein 1-25 methylene groups of L¹ are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently —OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen, or two R² form an oxo;
- - is a bond or absent;
one of X¹ or X² is N-Q-R³ and the other is CH or CH₂ as permitted by valency, provided that when X² is N-Q-R³, - - is absent;
Y¹, Y², and Y³ are each independently CH or N;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, or —C(O)O—(C₁₋₆ alkyl);
each R⁵ is independently a bond, a leaving group, a protecting group, H, alkynyl, aryl, or heteroaryl;
R^{X} is H, C₁₋₆ alkyl, C₁₋₆ haloalkyl, or C₁₋₆ hydroxyalkyl;
R^{Y} is C₃₋₈ cycloalkyl, C₃₋₈ hydroxycycloalkyl, aryl, or heterocycle;
a is an integer of 0-3; and
b is an integer of 0-2; and
the compound of formula (II) is not

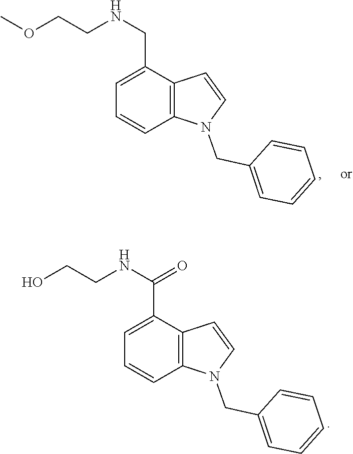 [SMILES: COCCNCc1cccc2c1ccn2Cc1ccccc1 ; O=C(NCCO)c1cccc2c1ccn2Cc1ccccc1]

16.-29. (canceled)

30. A compound of formula (III) 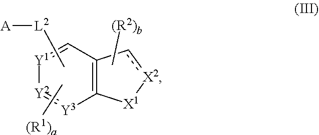
or a pharmaceutically acceptable salt, stereoisomer, or deuterated form thereof,
wherein:
A is a ligand that binds to a protein, a protein aggregate, a protein complex, or a lipid;
L² is C₂-C₅₀ alkylene, C₂-C₂₅ alkenylene, C₂-C₂₅ alkynylene, or —(C₂-C₅₀ alkylene)-arylene, wherein 1-25 methylene groups of L² are optionally and independently replaced by —N(H)—, —N(C₁-C₆ alkyl)-, —N(C₃-C₈ cycloalkyl)-, —O—, —C(O)—, —C(O)O—, —S—, —S(O)—, —S(O)₂—, —S(O)₂N(C₁-C₆ alkyl)-, —S(O)₂N(C₃-C₈ cycloalkyl)-, —N(H)C(O)—, —N(C₁-C₆ alkyl)C(O)—, —N(C₃-C₈ cycloalkyl)C(O)—, —N(H)C(O)N(H)—, —N(C₁-C₆ alkyl)C(O)N(H)—, —N(H)C(O)N(C₁-C₆ alkyl)-, —N(C₁-C₆ alkyl)C(O)N(C₁-C₆ alkyl)-, —C(O)N(H)—, —C(O)N(C₁-C₆ alkyl)-, —C(O)N(C₃-C₈ cycloalkyl)-, arylene, heteroarylene, heterocyclylene, C₃-C₈ cycloalkylene, or C₃-C₈ cycloalkenylene, wherein the alkylene, alkenylene, alkynylene, alkyl, arylene, heteroarylene, and heterocyclylene are each optionally and independently substituted with 1, 2, or 3 R^{Z}, wherein each R^{Z} is independently OH, C₁₋₆ alkyl, or halogen;
each R¹ is independently C₁₋₆ alkyl, C₃-C₈ cycloalkyl, or halogen;
each R² is independently C₁₋₆ alkyl, C₃-C₈ cycloalkyl, or halogen, or two R² form an oxo;
- - is a bond or absent;
one of X¹ or X² is N-Q-R³ and the other is CH or CH₂ as permitted by valency, provided that when X² is N-Q-R³, - - is absent;
Y¹, Y², and Y³ are each independently CH or N;
Q is absent, C₁₋₆ alkylene, —C₁₋₆ alkylene-C(O)—, C₃₋₈ cycloalkylene, —C₃₋₈ cycloalkylene-C(O)—, —C(O)—, or —S(O)₂—;
R³ is carbocycle, heterocycle, aryl, heteroaryl, or —NR^{X}R^{Y}, wherein the carbocycle, heterocycle, aryl, and heteroaryl are each optionally substituted with 1, 2, or 3 R⁴;
each R⁴ is independently halogen, —OH, C₁₋₆ alkyl, C₁₋₆ alkoxy, or —C(O)O—(C₁₋₆ alkyl);
R^{X} is H, C₁₋₆ alkyl, C₁₋₆ haloalkyl, or C₁₋₆ hydroxyalkyl;
R^{Y} is C₃₋₈ cycloalkyl, C₃₋₈ hydroxycycloalkyl, aryl, or heterocycle;
a is an integer of 0-3; and
b is an integer of 0-2.

31.-40. (canceled)

41. The compound of claim 30, or a pharmaceutically acceptable salt, a stereoisomer, or a deuterated form thereof, wherein:
the protein is a protein that is associated with cancer, optionally wherein the protein associated with cancer comprises a mutation or a fusion, and optionally wherein the protein associated with cancer is BRD4;
the protein is a protein associated with a metabolic disease:
the protein is a protein associated with inflammation:
the protein is present in bacteria:
the protein is present in a virus particle:
the protein is a mitochondrial protein:
the protein is an intracellular protein; or
the protein aggregate is a Tau protein aggregate, an alpha-synuclein protein aggregate, a β-sheet protein aggregate, a mutant Huntingtin protein aggregate, an amyloid protein aggregate, or a TDP-43 protein aggregate.

42.-52. (canceled)

53. The compound of claim 1, or a pharmaceutically acceptable salt, a stereoisomer, or a deuterated form thereof, wherein each R¹ is independent halogen.

54. (canceled)

55. The compound of claim 1, or a pharmaceutically acceptable salt, a stereoisomer, or a deuterated form thereof, wherein a is 0 or 1.

56. The compound of claim 1, or a pharmaceutically acceptable salt, a stereoisomer, or a deuterated form thereof, wherein each R² is independently C₁₋₆ alkyl.

57.-59. (canceled)

60. The compound of claim 1, or a pharmaceutically acceptable salt, a stereoisomer, or a deuterated form thereof, wherein Q is absent, C₁₋₄ alkylene, —(C₁₋₃ alkylene)C(O)—, C₃₋₆ cycloalkylene, —(C₃₋₆ cycloalkylene)C(O)—, —C(O)—, or —S(O)₂—.

61.-68. (canceled)

69. The compound of claim 1, or a pharmaceutically acceptable salt, a stereoisomer, or a deuterated form thereof, wherein R³ is C₃₋₈ carbocycle, 3-10 membered heterocycle, C₆₋₁₀ aryl, or 5-10 membered heteroaryl, and wherein the C₃₋₈ carbocycle, 3-8 membered heterocycle, C₆₋₁₀ aryl, and 5-10 membered heteroaryl are each optionally substituted with 1 or 2 R⁴, and each R⁴ is independently halogen, —OH, C₁₋₆ alkyl, C₁₋₆ alkoxy, or —C(O)O—C₁₋₆ alkyl.

70.-85. (canceled)

86. The compound of claim 1, wherein the compound of formula (I) is: 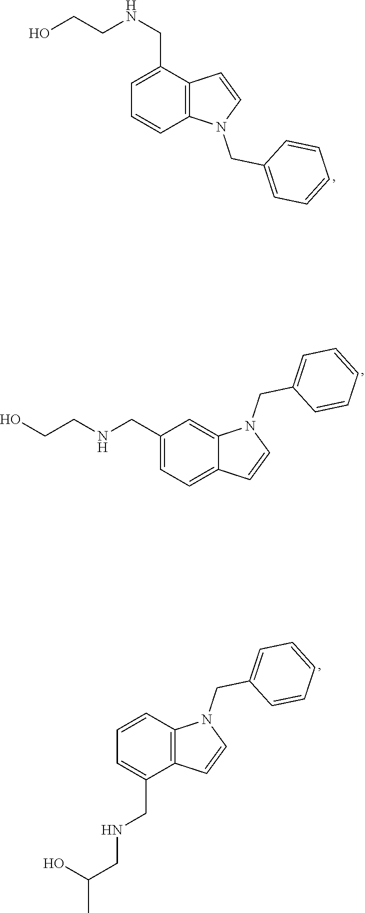 [SMILES: OCCNCc1cccc2c1ccn2Cc1ccccc1 ; OCCNCc1ccc2ccn(Cc3ccccc3)c2c1 ; CC(O)CNCc1cccc2c1ccn2Cc1ccccc1] 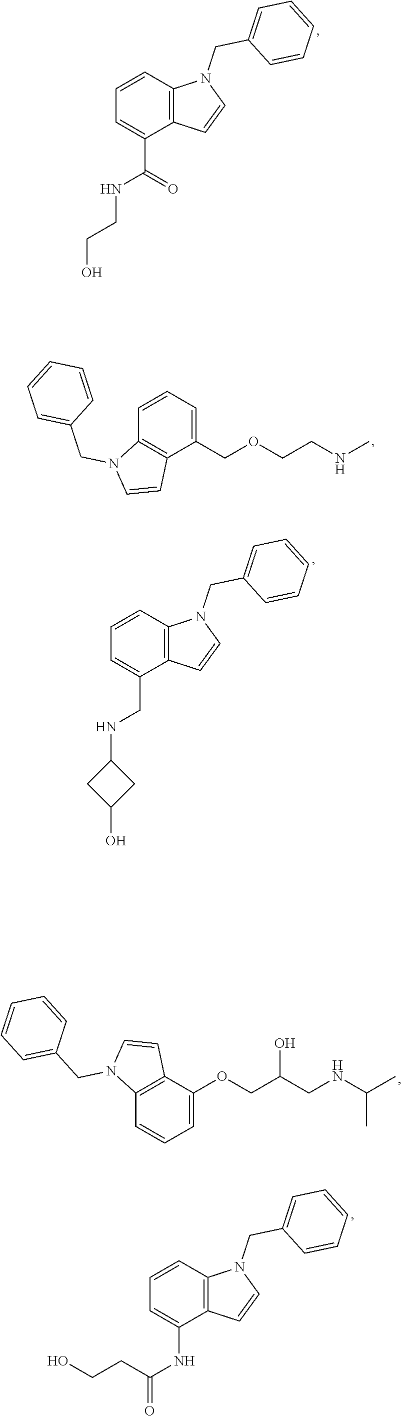 [SMILES: O=C(NCCO)c1cccc2c1ccn2Cc1ccccc1 ; CNCCOCc1cccc2c1ccn2Cc1ccccc1 ; OC1CC(NCc2cccc3c2ccn3Cc2ccccc2)C1 ; CC(C)NCC(O)COc1cccc2c1ccn2Cc1ccccc1 ; O=C(CCO)Nc1cccc2c1ccn2Cc1ccccc1] 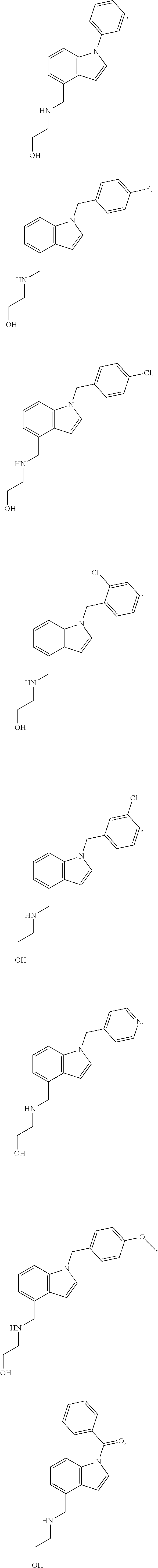 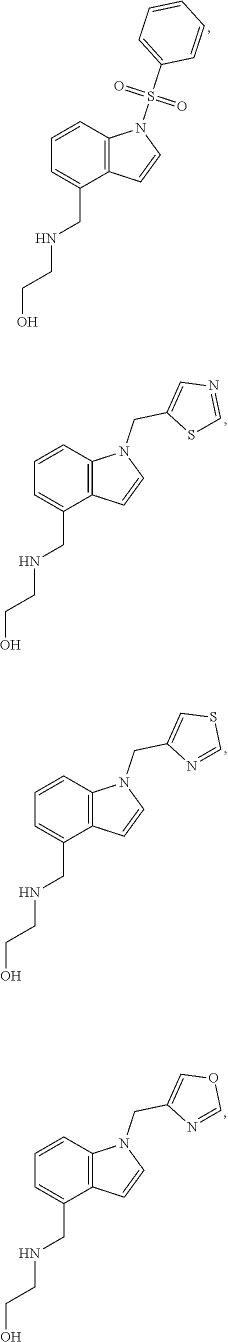 [SMILES: O=S(=O)(c1ccccc1)n1ccc2c(CNCCO)cccc21 ; OCCNCc1cccc2c1ccn2Cc1cncs1 ; OCCNCc1cccc2c1ccn2Cc1cscn1 ; OCCNCc1cccc2c1ccn2Cc1cocn1] 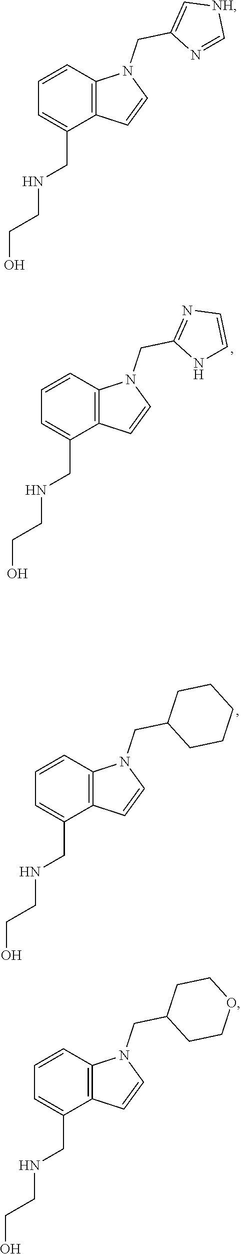 [SMILES: OCCNCc1cccc2c1ccn2Cc1c[nH]cn1 ; OCCNCc1cccc2c1ccn2Cc1ncc[nH]1 ; OCCNCc1cccc2c1ccn2CC1CCCCC1 ; OCCNCc1cccc2c1ccn2CC1CCOCC1] 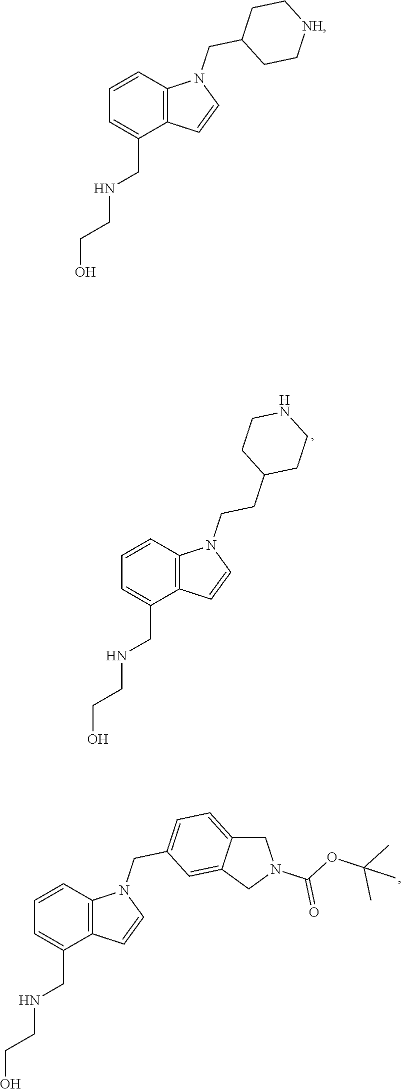 [SMILES: OCCNCc1cccc2c1ccn2CC1CCNCC1 ; OCCNCc1cccc2c1ccn2CCC1CCNCC1 ; CC(C)(C)OC(=O)N1Cc2ccc(Cn3ccc4c(CNCCO)cccc43)cc2C1] 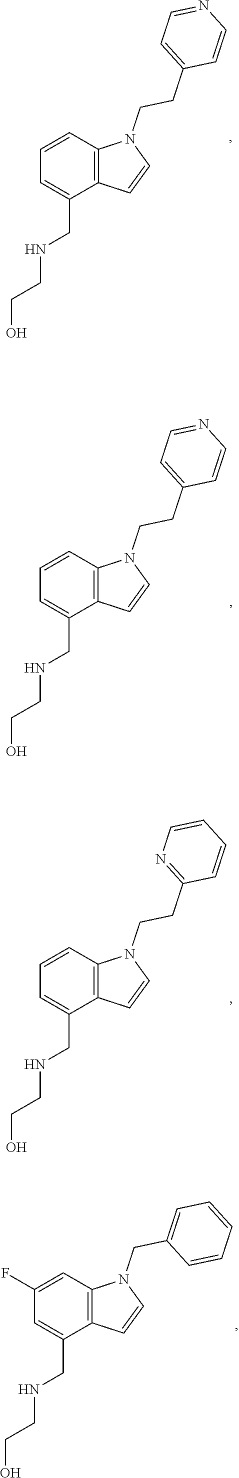 [SMILES: OCCNCc1cccc2c1ccn2CCc1ccncc1 ; OCCNCc1cccc2c1ccn2CCc1ccncc1 ; OCCNCc1cccc2c1ccn2CCc1ccccn1 ; OCCNCc1cc(F)cc2c1ccn2Cc1ccccc1] 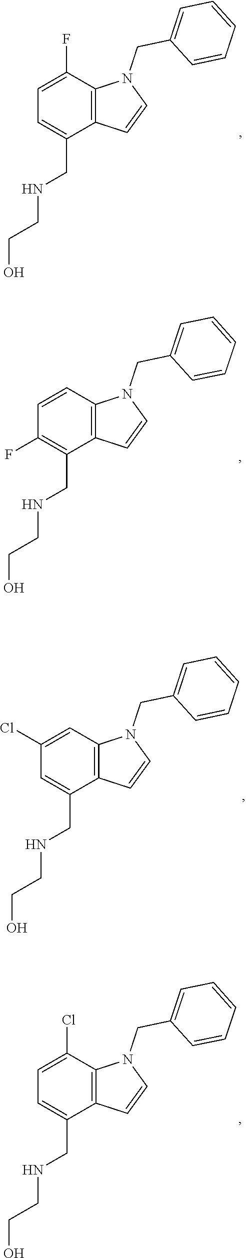 [SMILES: OCCNCc1ccc(F)c2c1ccn2Cc1ccccc1 ; OCCNCc1c(F)ccc2c1ccn2Cc1ccccc1 ; OCCNCc1cc(Cl)cc2c1ccn2Cc1ccccc1 ; OCCNCc1ccc(Cl)c2c1ccn2Cc1ccccc1] 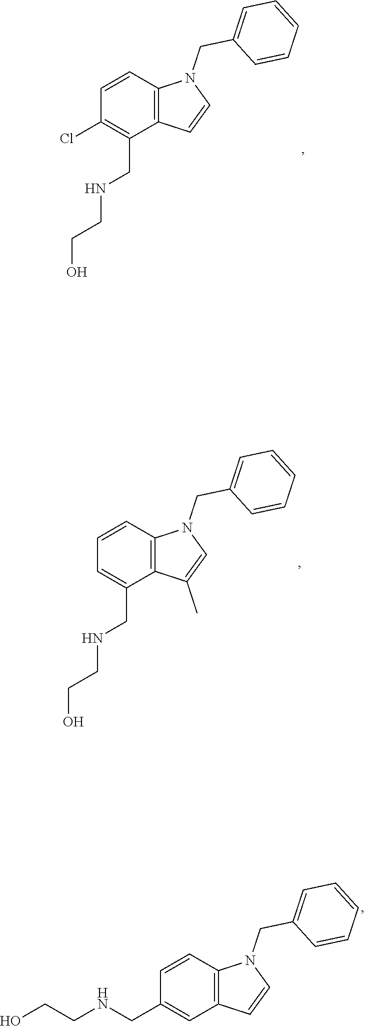 [SMILES: OCCNCc1c(Cl)ccc2c1ccn2Cc1ccccc1 ; Cc1cn(Cc2ccccc2)c2cccc(CNCCO)c12 ; OCCNCc1ccc2c(ccn2Cc2ccccc2)c1] 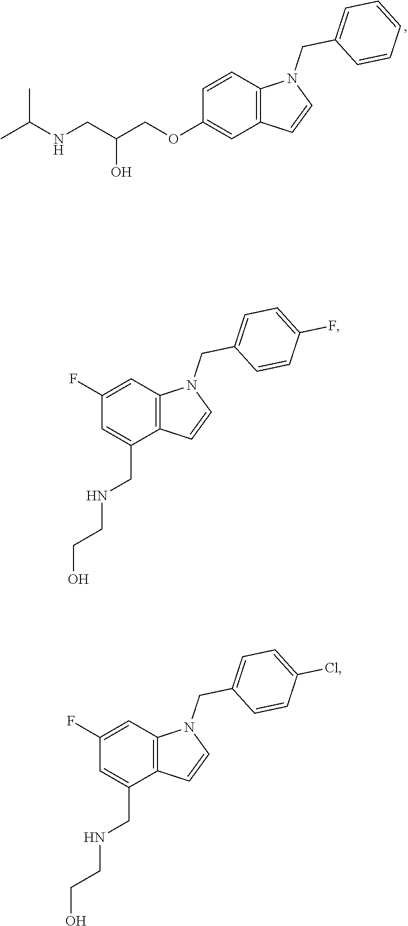 [SMILES: CC(C)NCC(O)COc1ccc2c(ccn2Cc2ccccc2)c1 ; OCCNCc1cc(F)cc2c1ccn2Cc1ccc(F)cc1 ; OCCNCc1cc(F)cc2c1ccn2Cc1ccc(Cl)cc1] 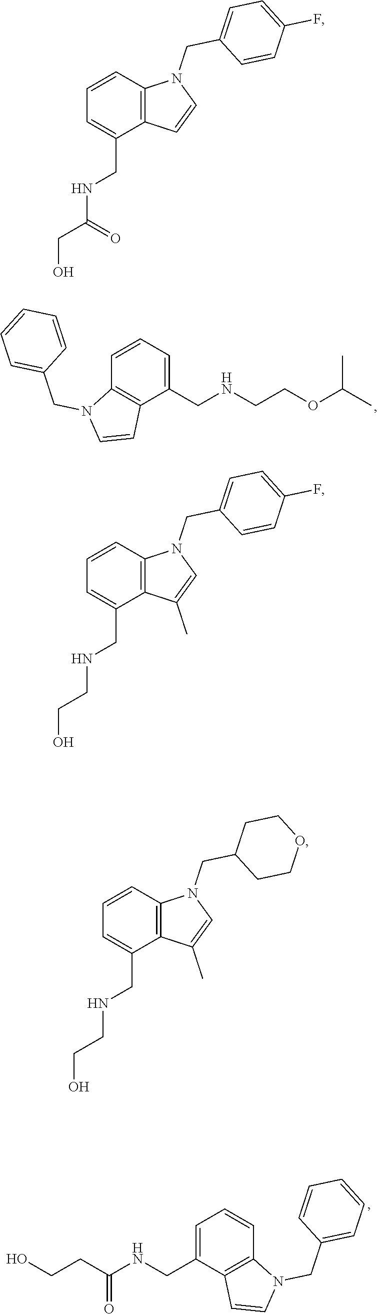 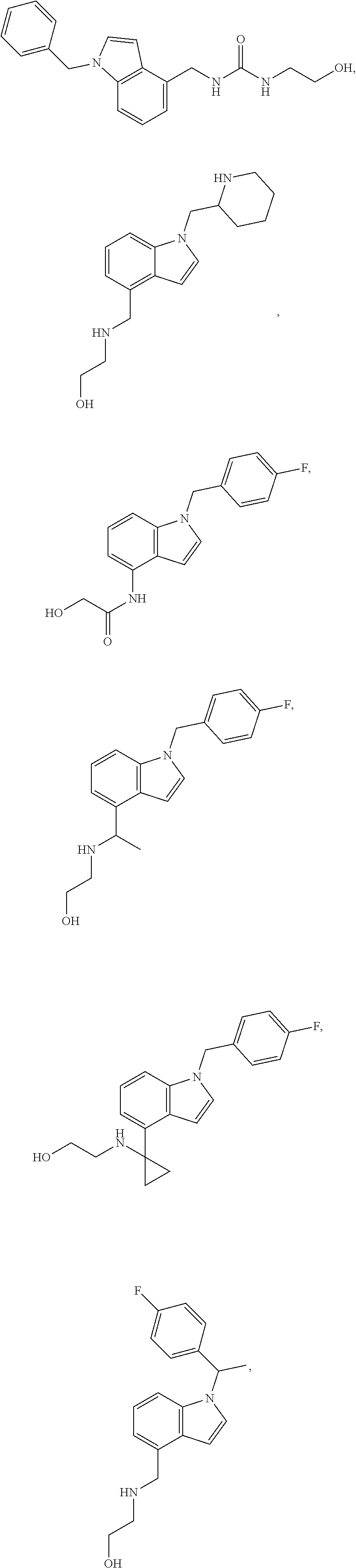 [SMILES: O=C(NCCO)NCc1cccc2c1ccn2Cc1ccccc1 ; OCCNCc1cccc2c1ccn2CC1CCCCN1 ; O=C(CO)Nc1cccc2c1ccn2Cc1ccc(F)cc1 ; CC(NCCO)c1cccc2c1ccn2Cc1ccc(F)cc1 ; OCCNC1(c2cccc3c2ccn3Cc2ccc(F)cc2)CC1 ; CC(c1ccc(F)cc1)n1ccc2c(CNCCO)cccc21] 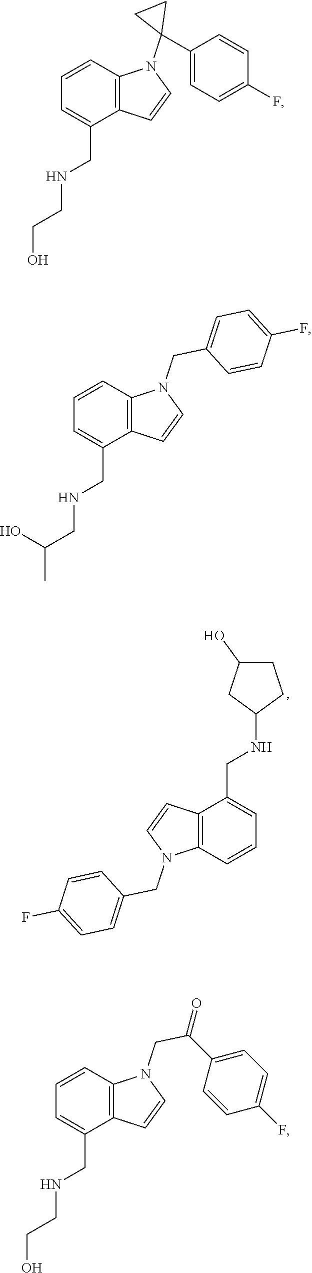 [SMILES: OCCNCc1cccc2c1ccn2C1(c2ccc(F)cc2)CC1 ; CC(O)CNCc1cccc2c1ccn2Cc1ccc(F)cc1 ; OC1CCC(NCc2cccc3c2ccn3Cc2ccc(F)cc2)C1 ; O=C(Cn1ccc2c(CNCCO)cccc21)c1ccc(F)cc1] 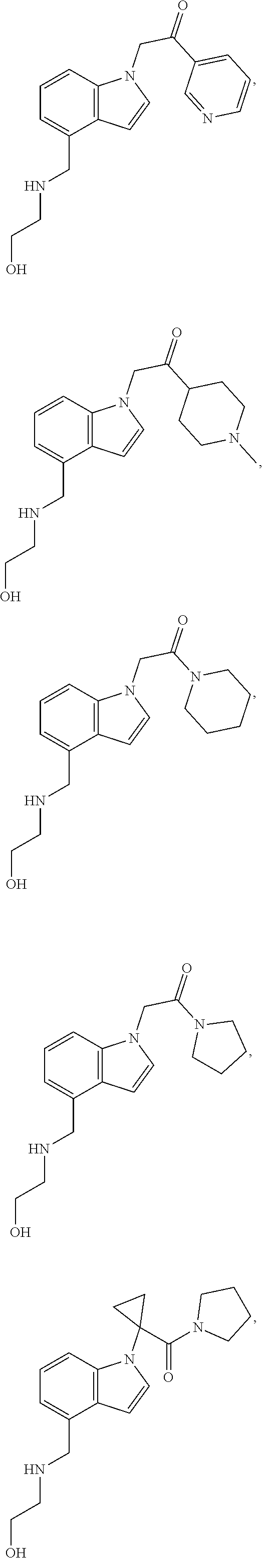 [SMILES: O=C(Cn1ccc2c(CNCCO)cccc21)c1cccnc1 ; CN1CCC(C(=O)Cn2ccc3c(CNCCO)cccc32)CC1 ; O=C(Cn1ccc2c(CNCCO)cccc21)N1CCCCC1 ; O=C(Cn1ccc2c(CNCCO)cccc21)N1CCCC1 ; O=C(N1CCCC1)C1(n2ccc3c(CNCCO)cccc32)CC1] 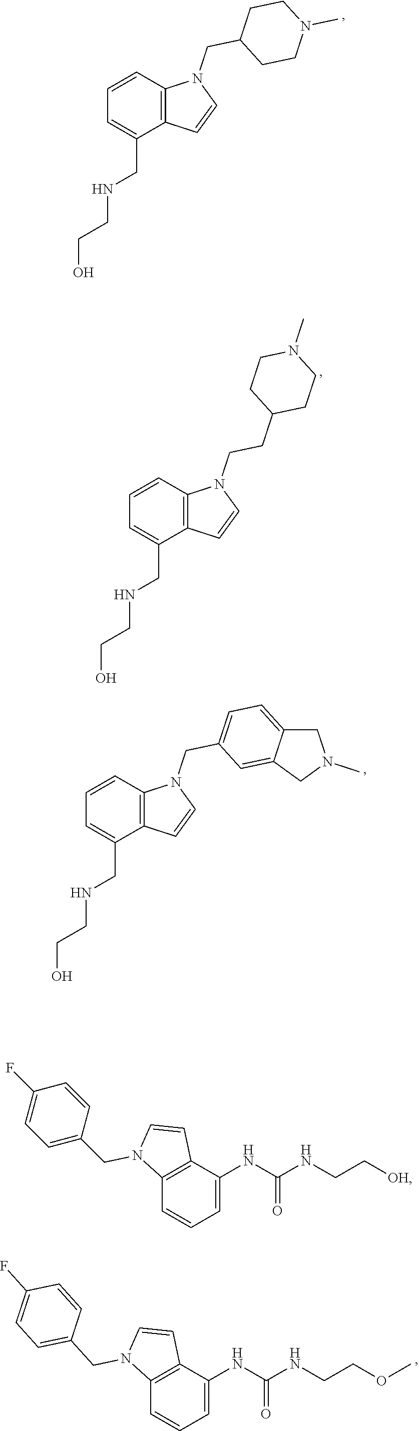 [SMILES: CN1CCC(Cn2ccc3c(CNCCO)cccc32)CC1 ; CN1CCC(CCn2ccc3c(CNCCO)cccc32)CC1 ; CN1Cc2ccc(Cn3ccc4c(CNCCO)cccc43)cc2C1 ; O=C(NCCO)Nc1cccc2c1ccn2Cc1ccc(F)cc1 ; COCCNC(=O)Nc1cccc2c1ccn2Cc1ccc(F)cc1] 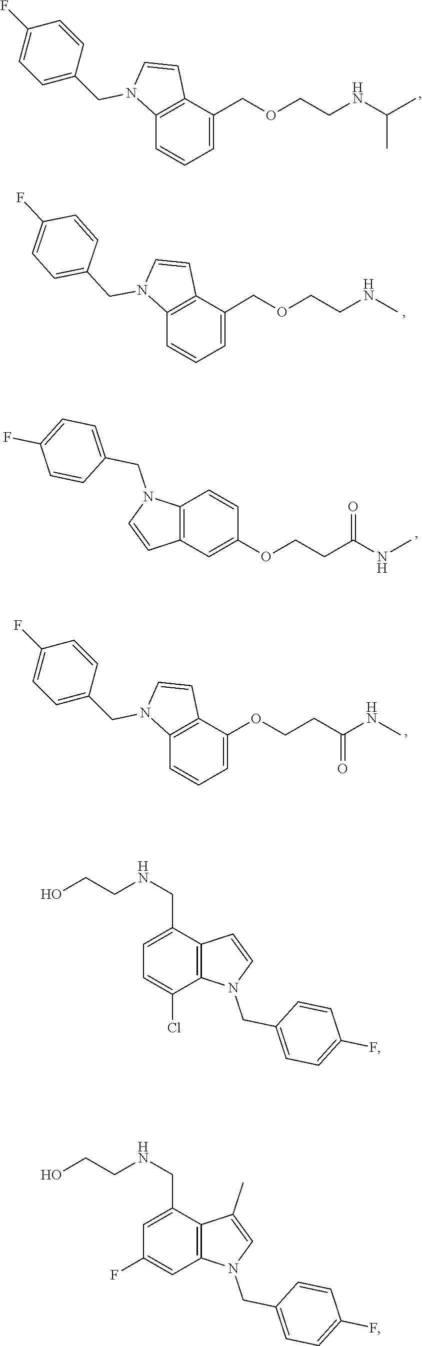 [SMILES: CC(C)NCCOCc1cccc2c1ccn2Cc1ccc(F)cc1 ; CNCCOCc1cccc2c1ccn2Cc1ccc(F)cc1 ; CNC(=O)CCOc1ccc2c(ccn2Cc2ccc(F)cc2)c1 ; CNC(=O)CCOc1cccc2c1ccn2Cc1ccc(F)cc1 ; OCCNCc1ccc(Cl)c2c1ccn2Cc1ccc(F)cc1 ; Cc1cn(Cc2ccc(F)cc2)c2cc(F)cc(CNCCO)c12] 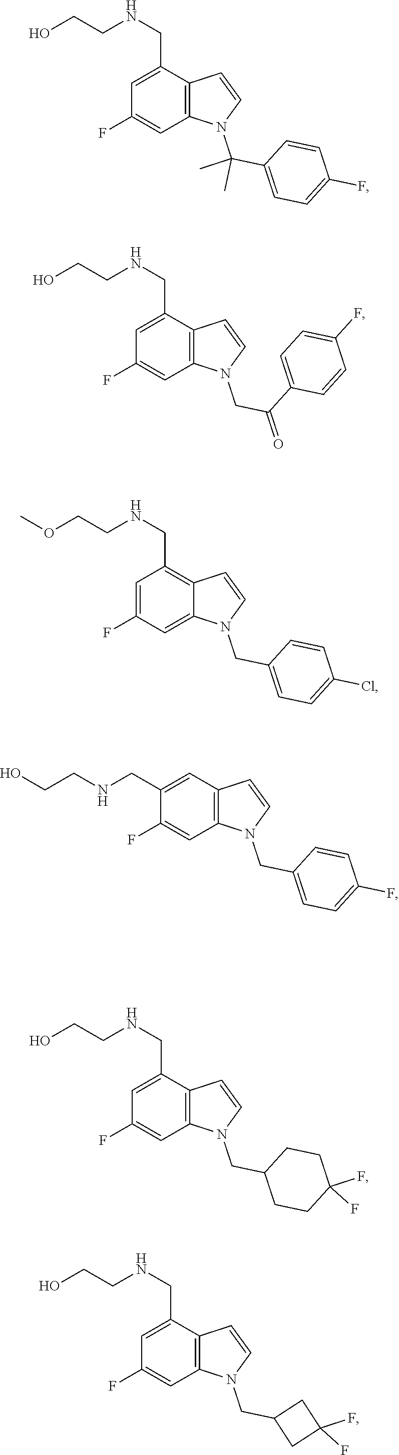 [SMILES: CC(C)(c1ccc(F)cc1)n1ccc2c(CNCCO)cc(F)cc21 ; O=C(Cn1ccc2c(CNCCO)cc(F)cc21)c1ccc(F)cc1 ; COCCNCc1cc(F)cc2c1ccn2Cc1ccc(Cl)cc1 ; OCCNCc1cc2ccn(Cc3ccc(F)cc3)c2cc1F ; OCCNCc1cc(F)cc2c1ccn2CC1CCC(F)(F)CC1 ; OCCNCc1cc(F)cc2c1ccn2CC1CC(F)(F)C1] 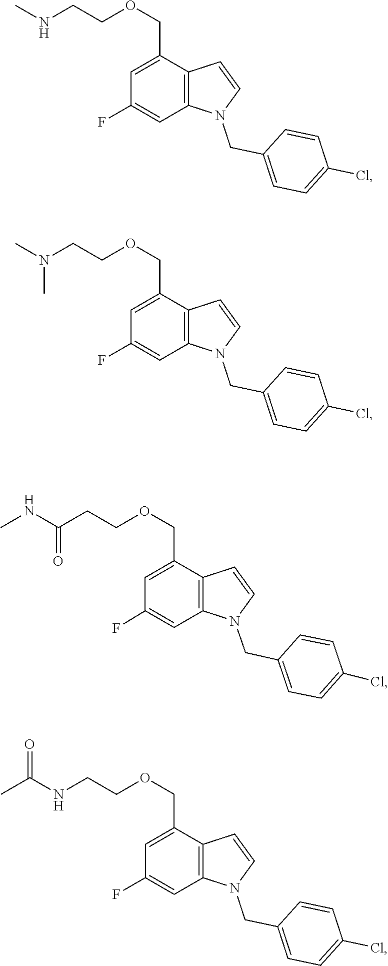 [SMILES: CNCCOCc1cc(F)cc2c1ccn2Cc1ccc(Cl)cc1 ; CN(C)CCOCc1cc(F)cc2c1ccn2Cc1ccc(Cl)cc1 ; CNC(=O)CCOCc1cc(F)cc2c1ccn2Cc1ccc(Cl)cc1 ; CC(=O)NCCOCc1cc(F)cc2c1ccn2Cc1ccc(Cl)cc1] 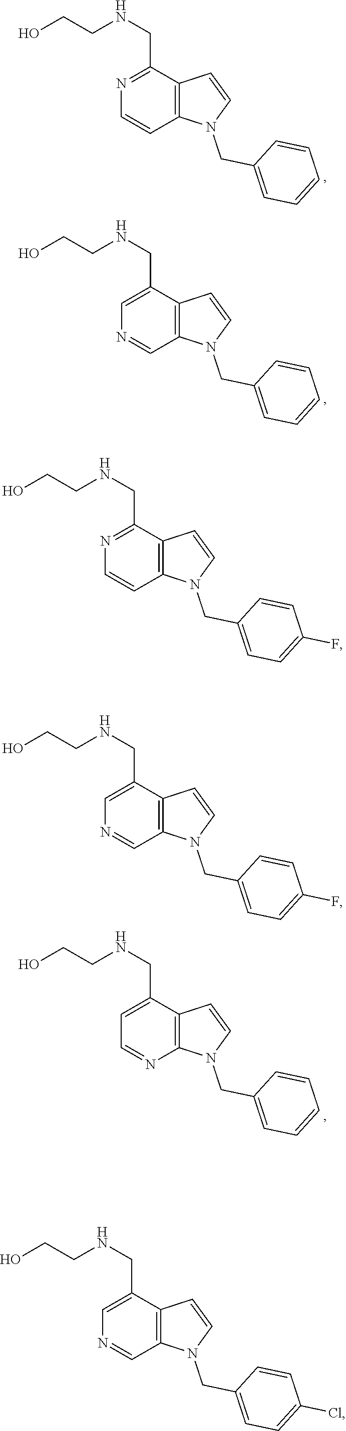 [SMILES: OCCNCc1nccc2c1ccn2Cc1ccccc1 ; OCCNCc1cncc2c1ccn2Cc1ccccc1 ; OCCNCc1nccc2c1ccn2Cc1ccc(F)cc1 ; OCCNCc1cncc2c1ccn2Cc1ccc(F)cc1 ; OCCNCc1ccnc2c1ccn2Cc1ccccc1 ; OCCNCc1cncc2c1ccn2Cc1ccc(Cl)cc1] 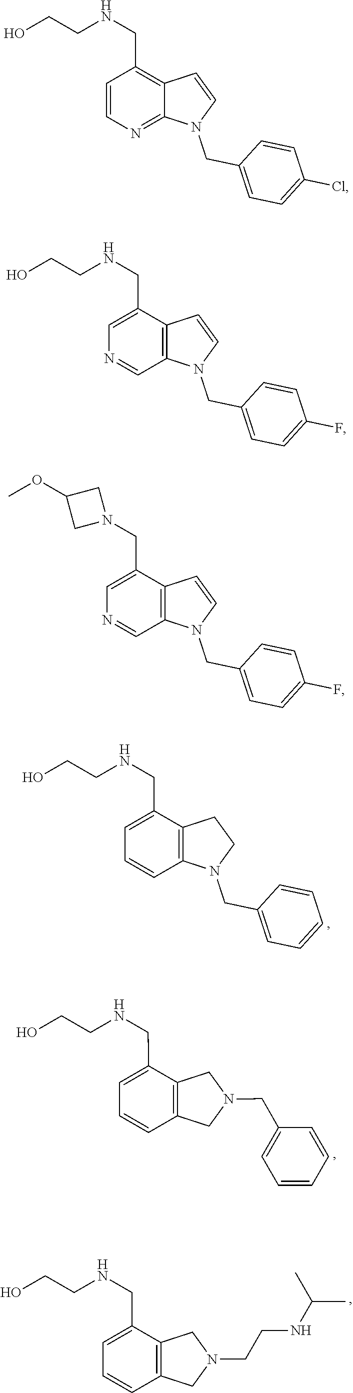 [SMILES: OCCNCc1ccnc2c1ccn2Cc1ccc(Cl)cc1 ; OCCNCc1cncc2c1ccn2Cc1ccc(F)cc1 ; COC1CN(Cc2cncc3c2ccn3Cc2ccc(F)cc2)C1 ; OCCNCc1cccc2c1CCN2Cc1ccccc1 ; OCCNCc1cccc2c1CN(Cc1ccccc1)C2 ; CC(C)NCCN1Cc2cccc(CNCCO)c2C1] 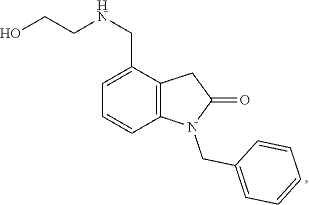 [SMILES: O=C1Cc2c(CNCCO)cccc2N1Cc1ccccc1]
or a pharmaceutically acceptable salt, a stereoisomer, or deuterated form thereof.

87.-88. (canceled)

89. The compound of claim 15, wherein the compound of formula (II) is 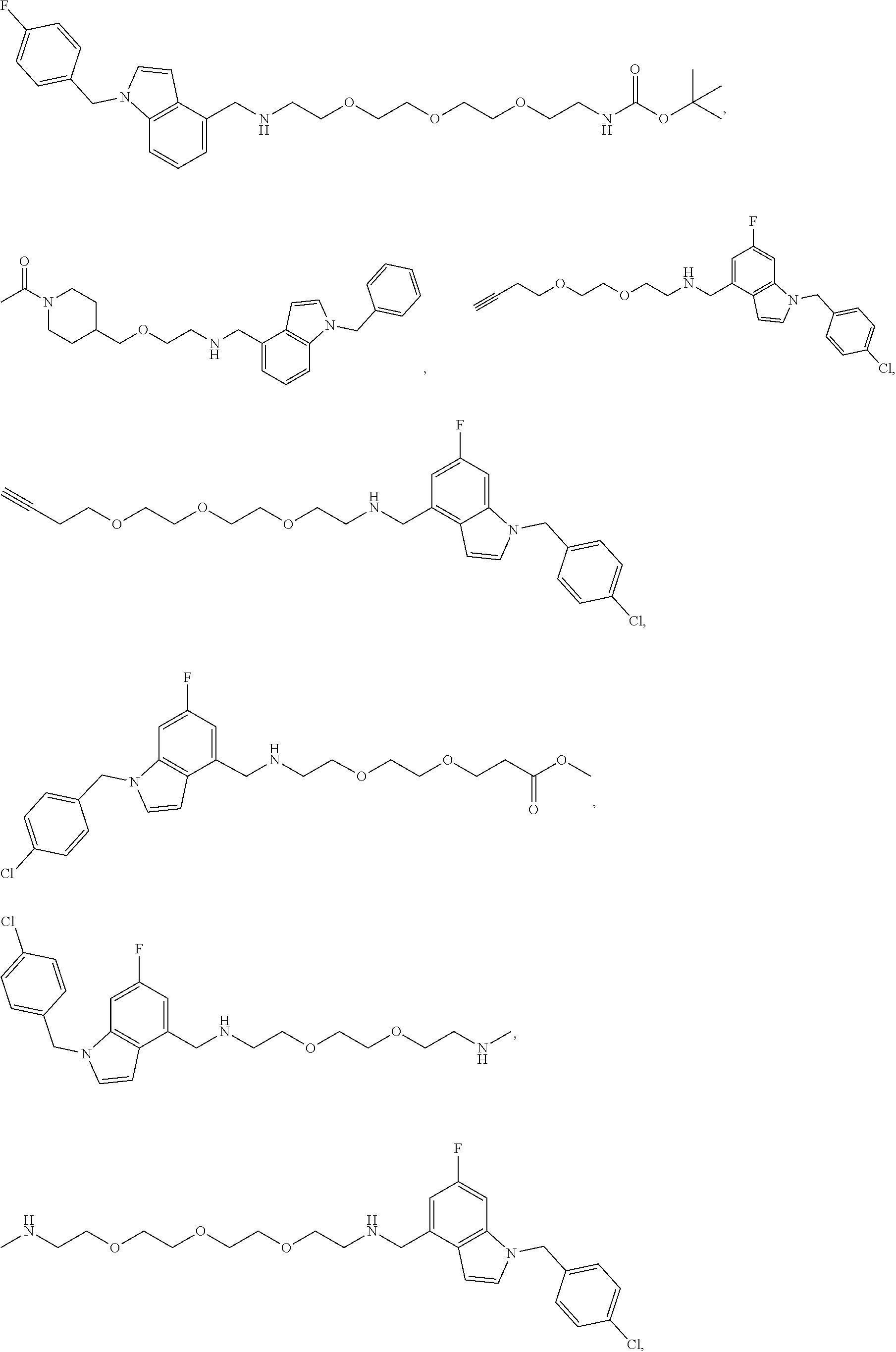 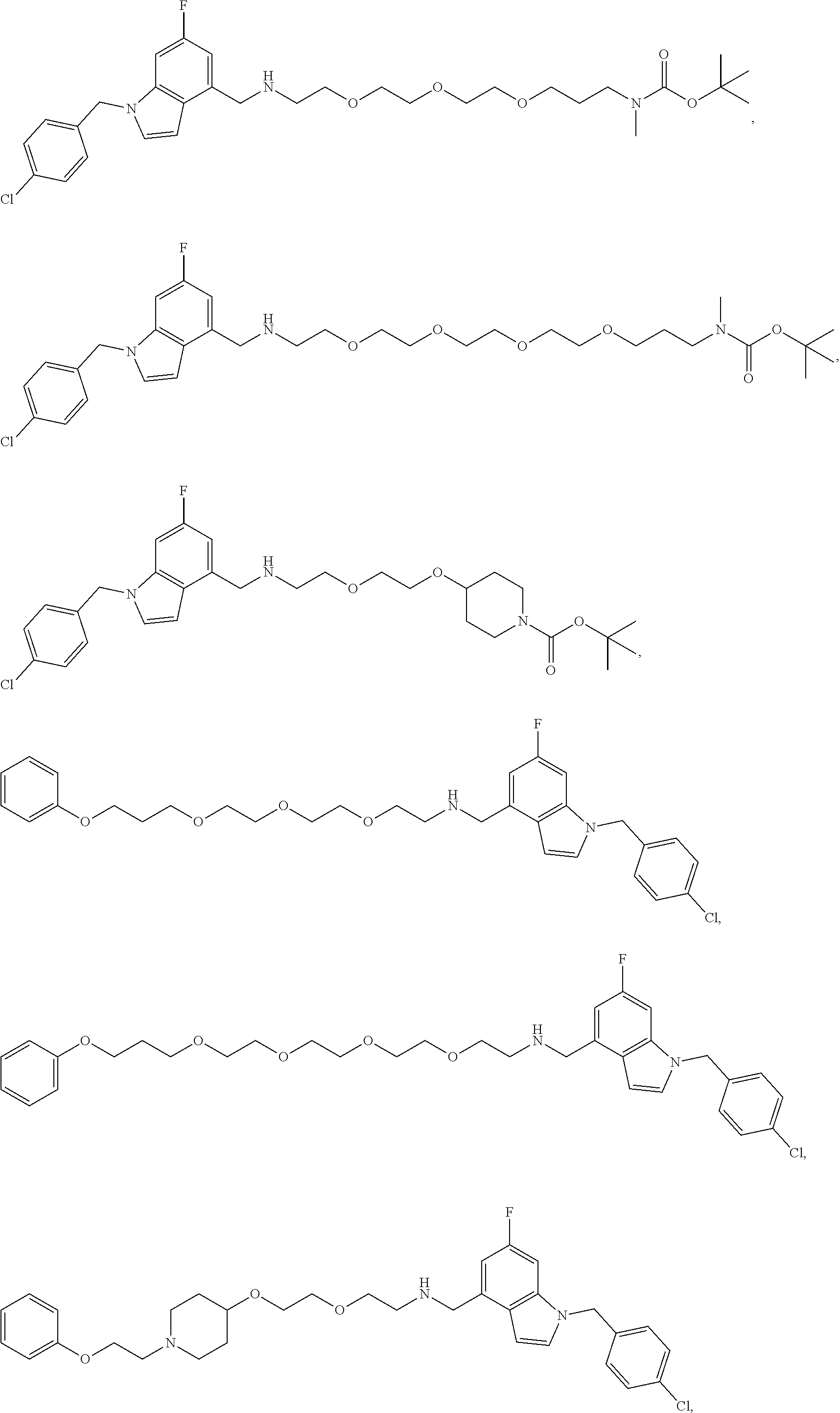 [SMILES: CN(CCCOCCOCCOCCNCc1cc(F)cc2c1ccn2Cc1ccc(Cl)cc1)C(=O)OC(C)(C)C ; CN(CCCOCCOCCOCCOCCNCc1cc(F)cc2c1ccn2Cc1ccc(Cl)cc1)C(=O)OC(C)(C)C ; CC(C)(C)OC(=O)N1CCC(OCCOCCNCc2cc(F)cc3c2ccn3Cc2ccc(Cl)cc2)CC1 ; Fc1cc(CNCCOCCOCCOCCCOc2ccccc2)c2ccn(Cc3ccc(Cl)cc3)c2c1 ; Fc1cc(CNCCOCCOCCOCCOCCCOc2ccccc2)c2ccn(Cc3ccc(Cl)cc3)c2c1 ; Fc1cc(CNCCOCCOC2CCN(CCOc3ccccc3)CC2)c2ccn(Cc3ccc(Cl)cc3)c2c1] 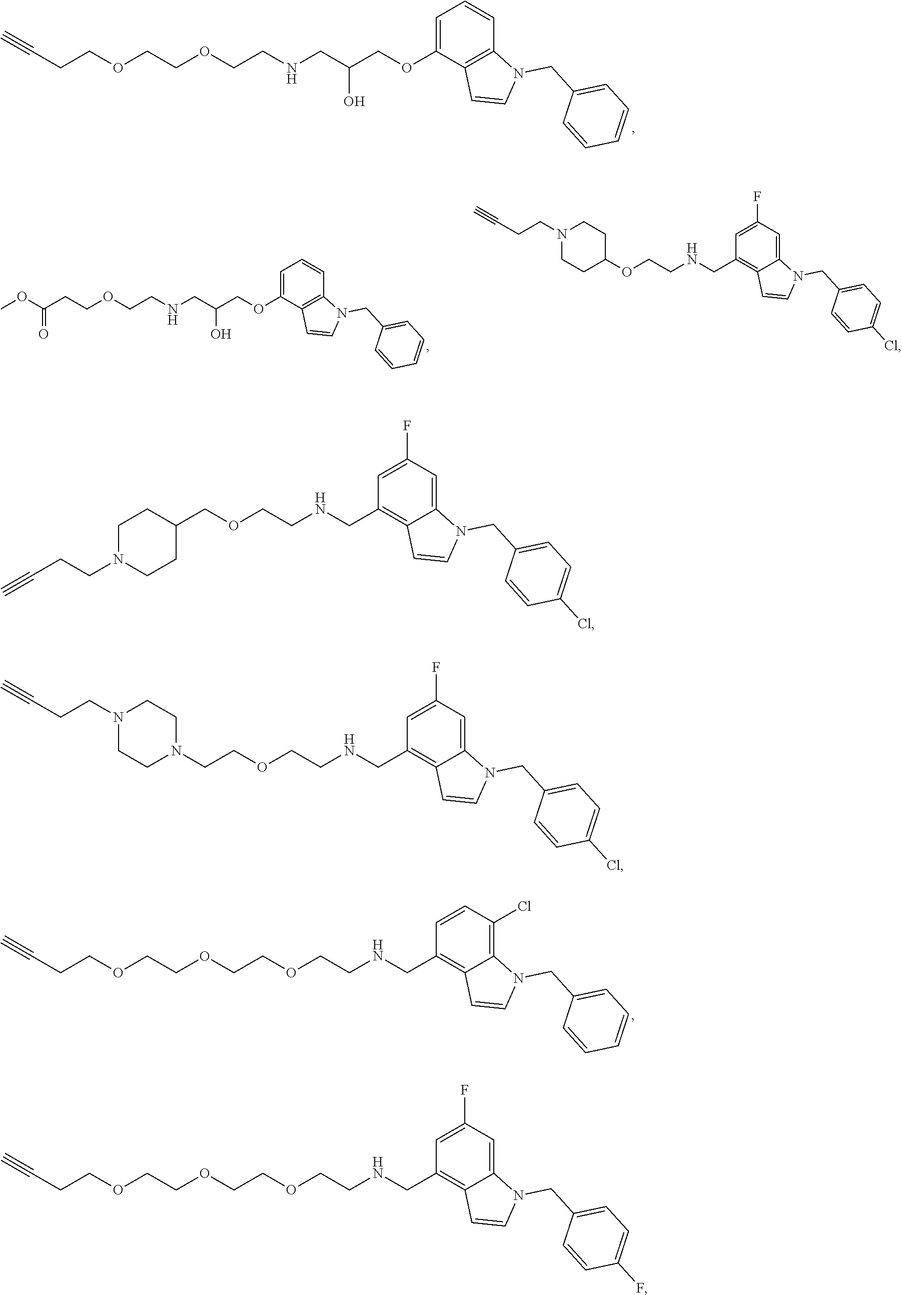 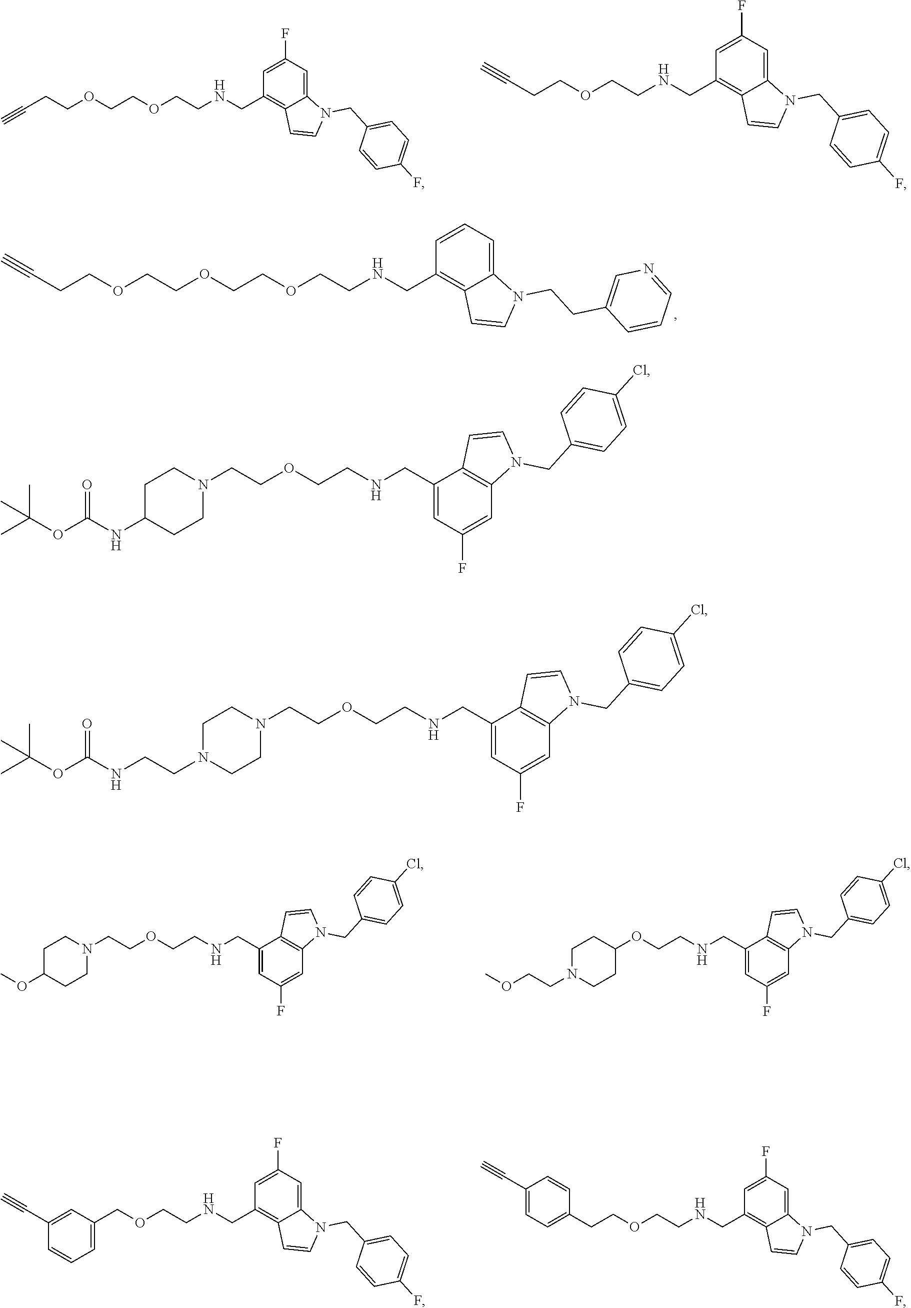 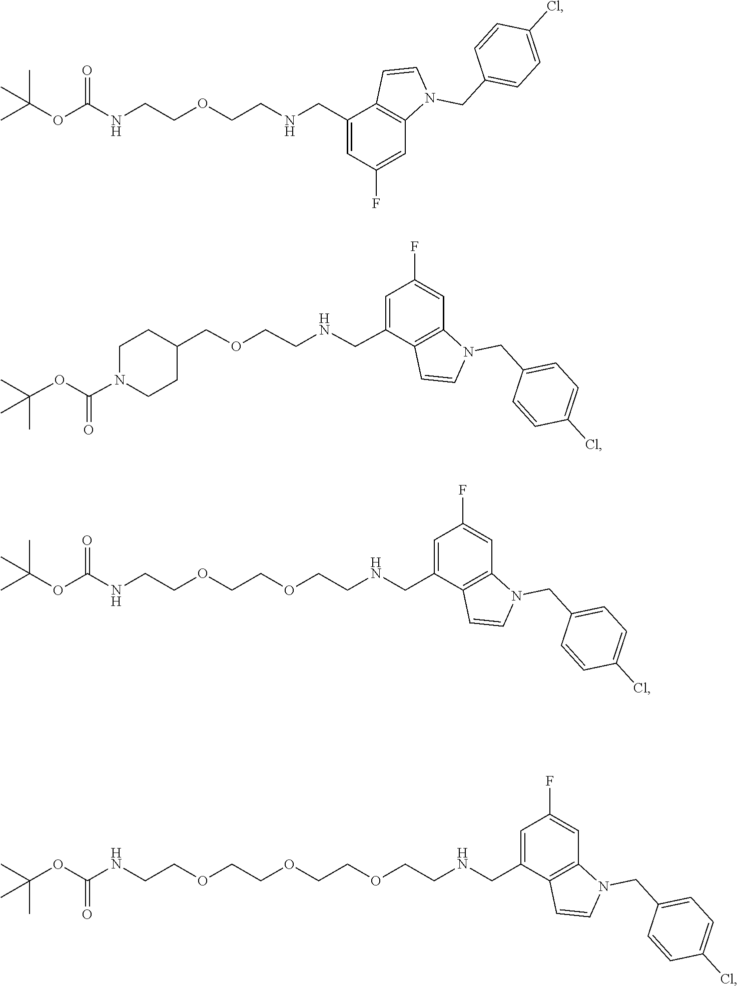 [SMILES: CC(C)(C)OC(=O)NCCOCCNCc1cc(F)cc2c1ccn2Cc1ccc(Cl)cc1 ; CC(C)(C)OC(=O)N1CCC(COCCNCc2cc(F)cc3c2ccn3Cc2ccc(Cl)cc2)CC1 ; CC(C)(C)OC(=O)NCCOCCOCCNCc1cc(F)cc2c1ccn2Cc1ccc(Cl)cc1 ; CC(C)(C)OC(=O)NCCOCCOCCOCCNCc1cc(F)cc2c1ccn2Cc1ccc(Cl)cc1] 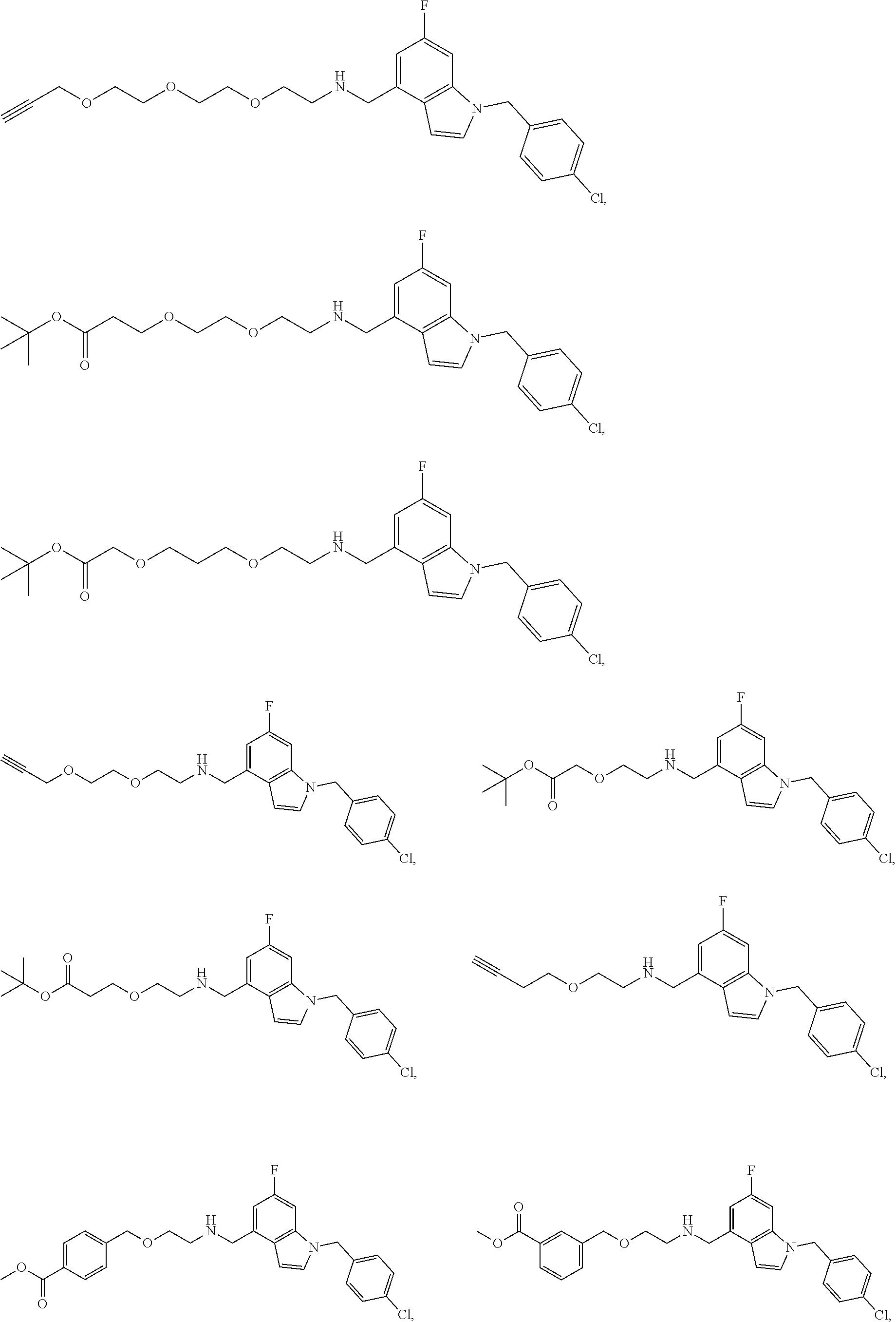 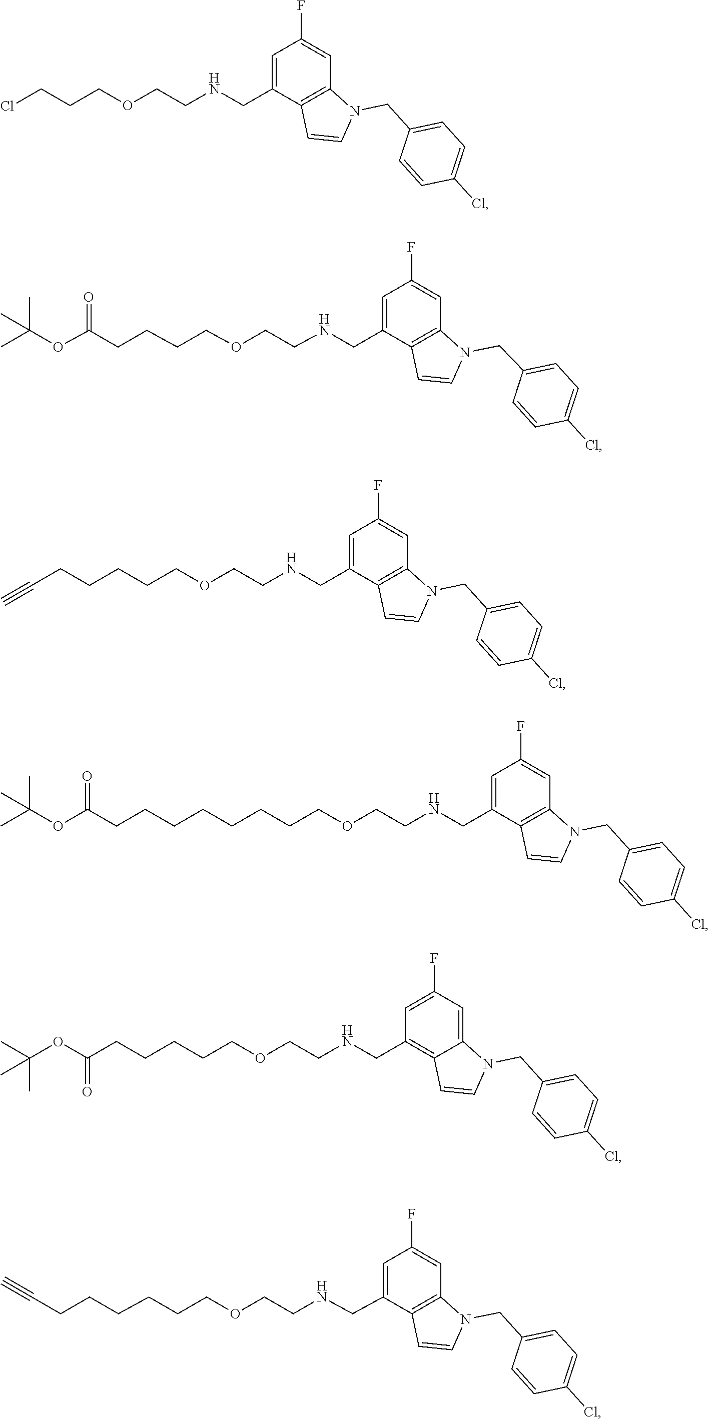 [SMILES: Fc1cc(CNCCOCCCCl)c2ccn(Cc3ccc(Cl)cc3)c2c1 ; CC(C)(C)OC(=O)CCCCOCCNCc1cc(F)cc2c1ccn2Cc1ccc(Cl)cc1 ; C#CCCCCCOCCNCc1cc(F)cc2c1ccn2Cc1ccc(Cl)cc1 ; CC(C)(C)OC(=O)CCCCCCCCOCCNCc1cc(F)cc2c1ccn2Cc1ccc(Cl)cc1 ; CC(C)(C)OC(=O)CCCCCOCCNCc1cc(F)cc2c1ccn2Cc1ccc(Cl)cc1 ; C#CCCCCCCOCCNCc1cc(F)cc2c1ccn2Cc1ccc(Cl)cc1] 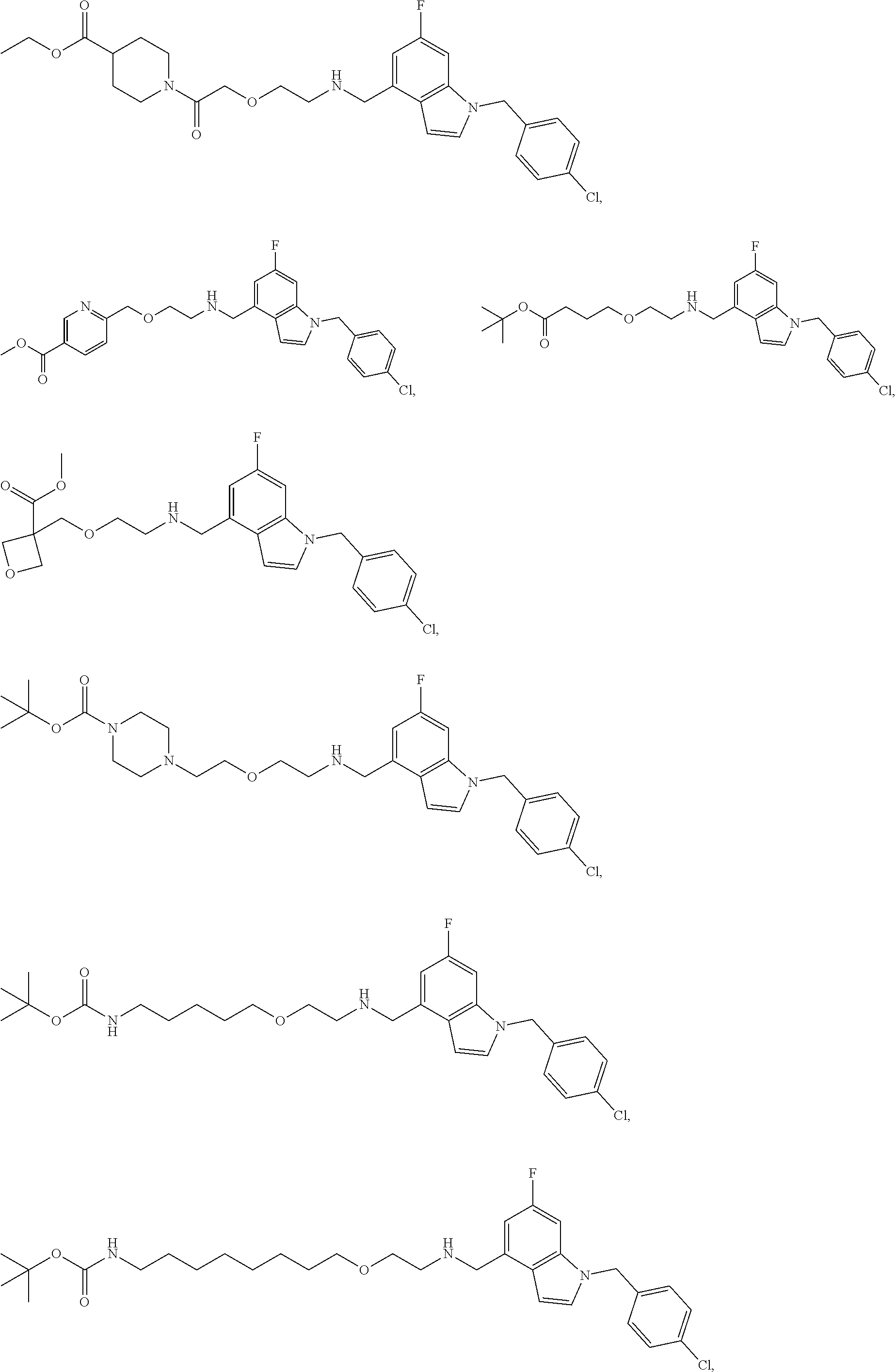 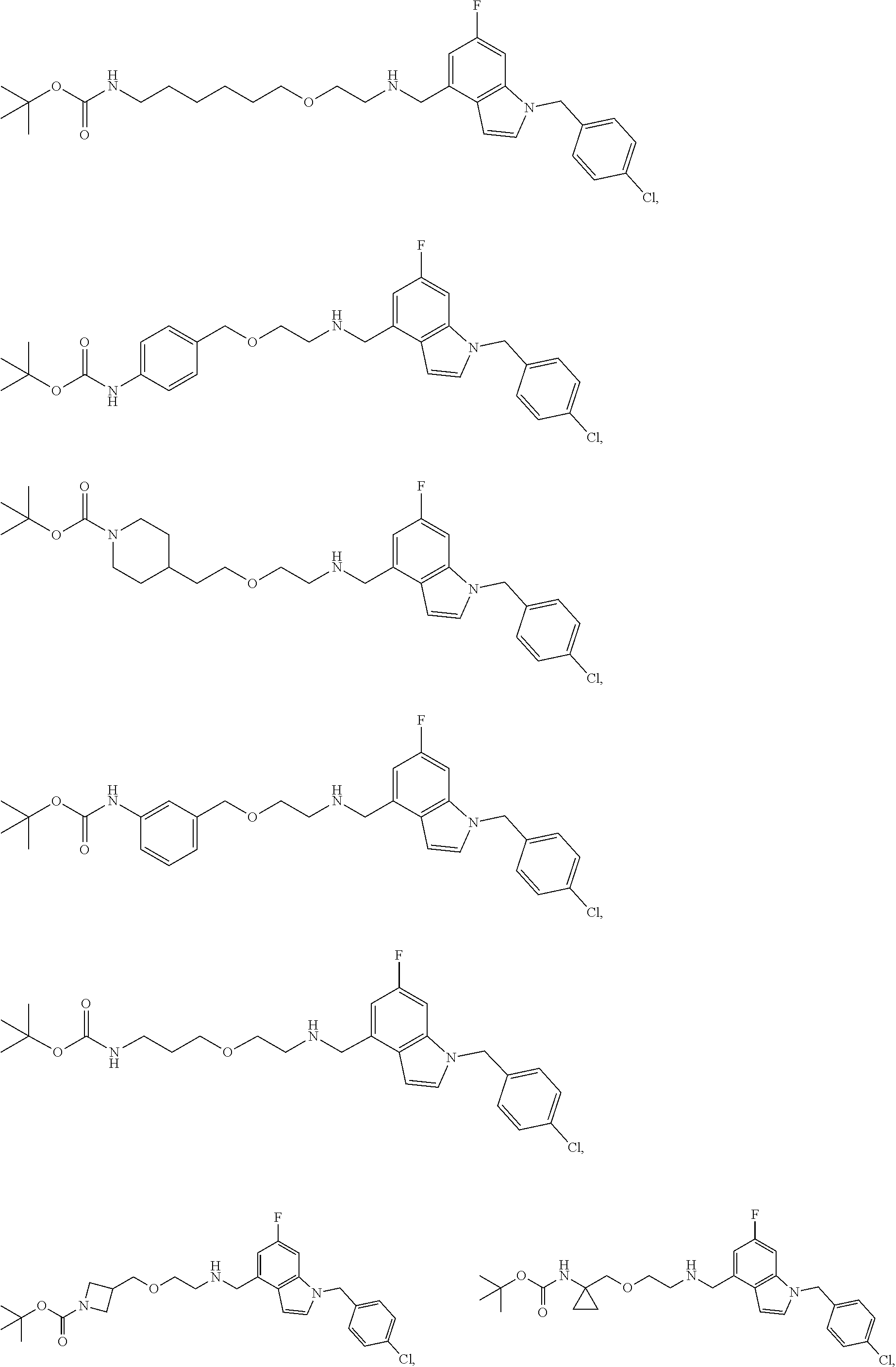 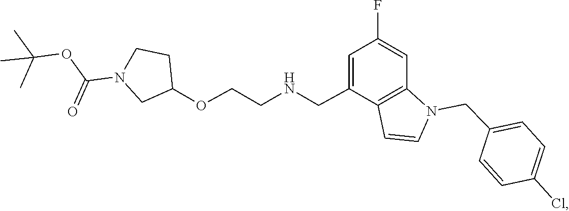 [SMILES: CC(C)(C)OC(=O)N1CCC(OCCNCc2cc(F)cc3c2ccn3Cc2ccc(Cl)cc2)C1] 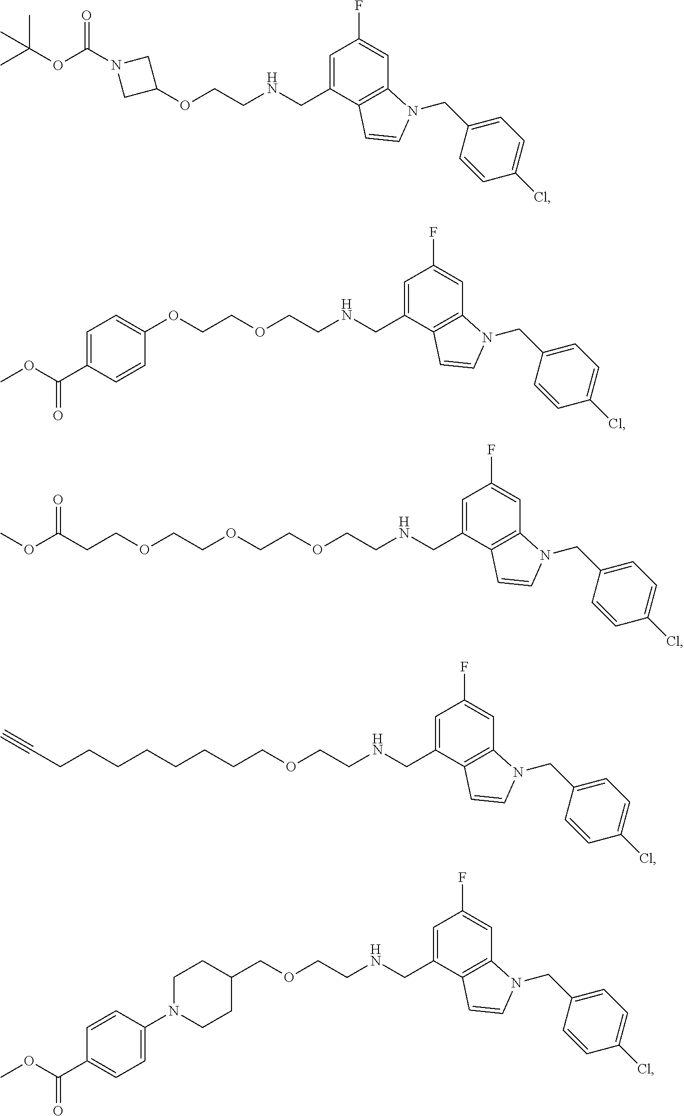 [SMILES: CC(C)(C)OC(=O)N1CC(OCCNCc2cc(F)cc3c2ccn3Cc2ccc(Cl)cc2)C1 ; COC(=O)c1ccc(OCCOCCNCc2cc(F)cc3c2ccn3Cc2ccc(Cl)cc2)cc1 ; COC(=O)CCOCCOCCOCCNCc1cc(F)cc2c1ccn2Cc1ccc(Cl)cc1 ; C#CCCCCCCCCOCCNCc1cc(F)cc2c1ccn2Cc1ccc(Cl)cc1 ; COC(=O)c1ccc(N2CCC(COCCNCc3cc(F)cc4c3ccn4Cc3ccc(Cl)cc3)CC2)cc1] 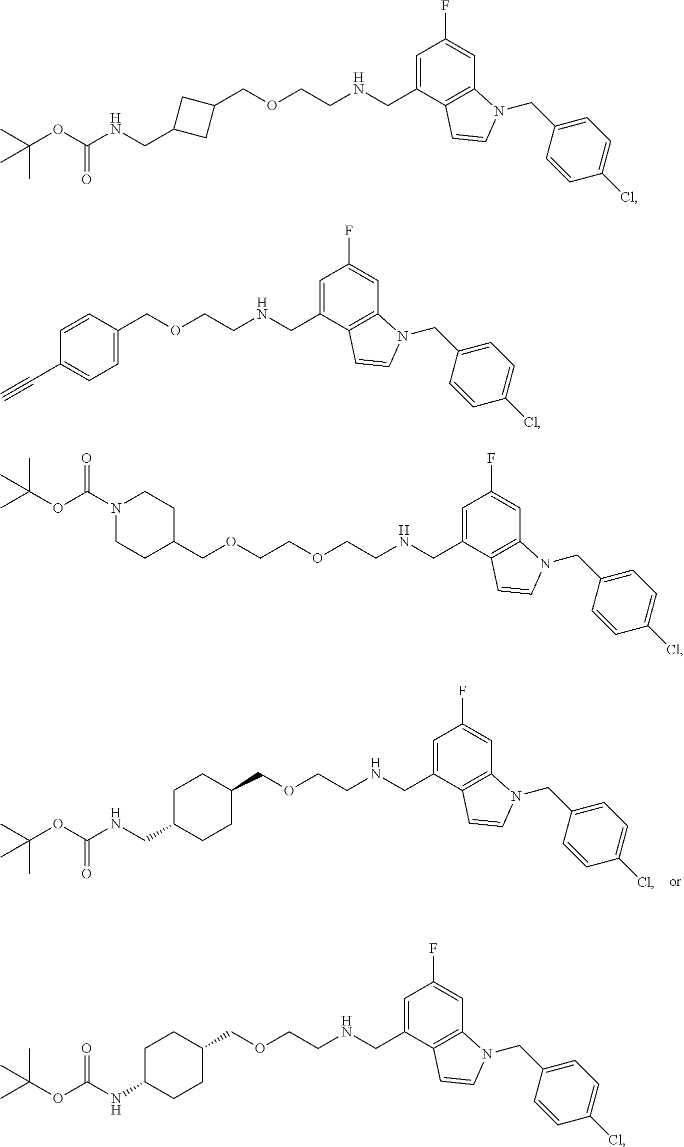 [SMILES: CC(C)(C)OC(=O)NCC1CC(COCCNCc2cc(F)cc3c2ccn3Cc2ccc(Cl)cc2)C1 ; C#Cc1ccc(COCCNCc2cc(F)cc3c2ccn3Cc2ccc(Cl)cc2)cc1 ; CC(C)(C)OC(=O)N1CCC(COCCOCCNCc2cc(F)cc3c2ccn3Cc2ccc(Cl)cc2)CC1 ; CC(C)(C)OC(=O)NC[C@H]1CC[C@H](COCCNCc2cc(F)cc3c2ccn3Cc2ccc(Cl)cc2)CC1 ; CC(C)(C)OC(=O)N[C@H]1CC[C@@H](COCCNCc2cc(F)cc3c2ccn3Cc2ccc(Cl)cc2)CC1]
or a pharmaceutically acceptable salt, a stereoisomer, or deuterated form thereof.

90. The compound of claim 30, wherein the compound of formula (III) is 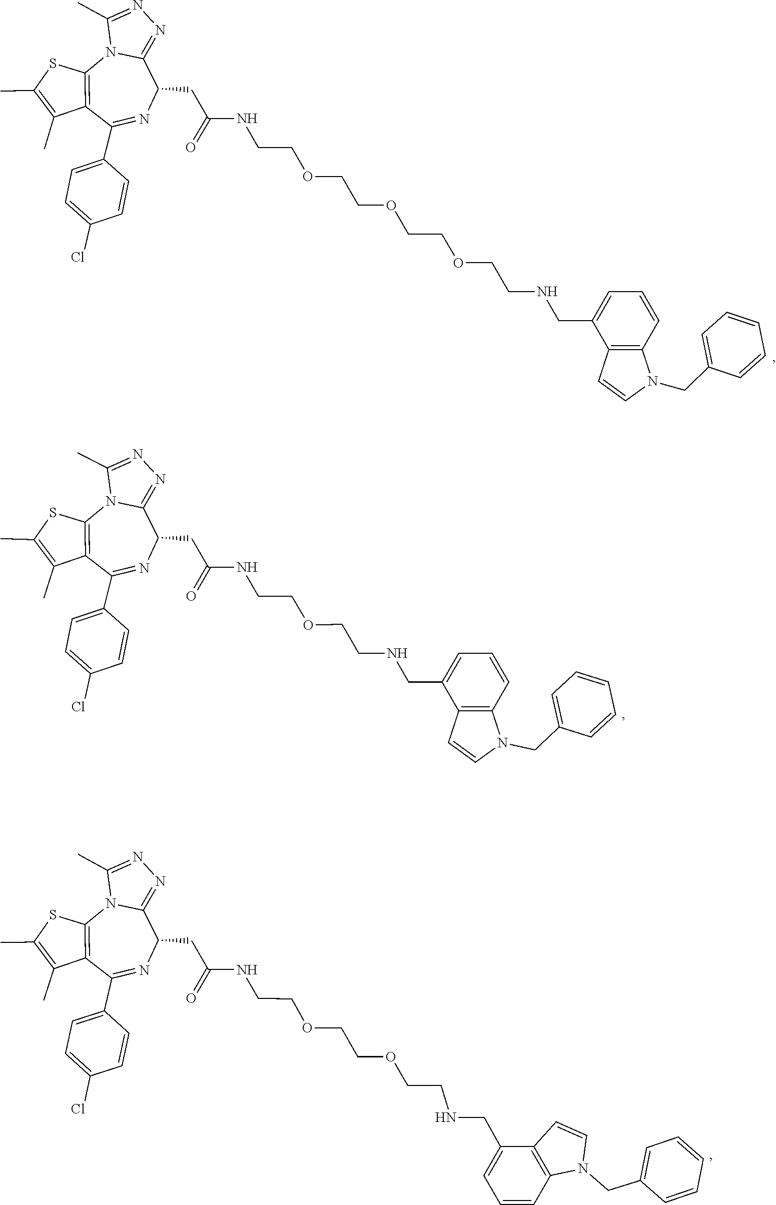 [SMILES: Cc1sc2c(c1C)C(c1ccc(Cl)cc1)=N[C@@H](CC(=O)NCCOCCOCCOCCNCc1cccc3c1ccn3Cc1ccccc1)c1nnc(C)n1-2 ; Cc1sc2c(c1C)C(c1ccc(Cl)cc1)=N[C@@H](CC(=O)NCCOCCNCc1cccc3c1ccn3Cc1ccccc1)c1nnc(C)n1-2 ; Cc1sc2c(c1C)C(c1ccc(Cl)cc1)=N[C@@H](CC(=O)NCCOCCOCCNCc1cccc3c1ccn3Cc1ccccc1)c1nnc(C)n1-2] 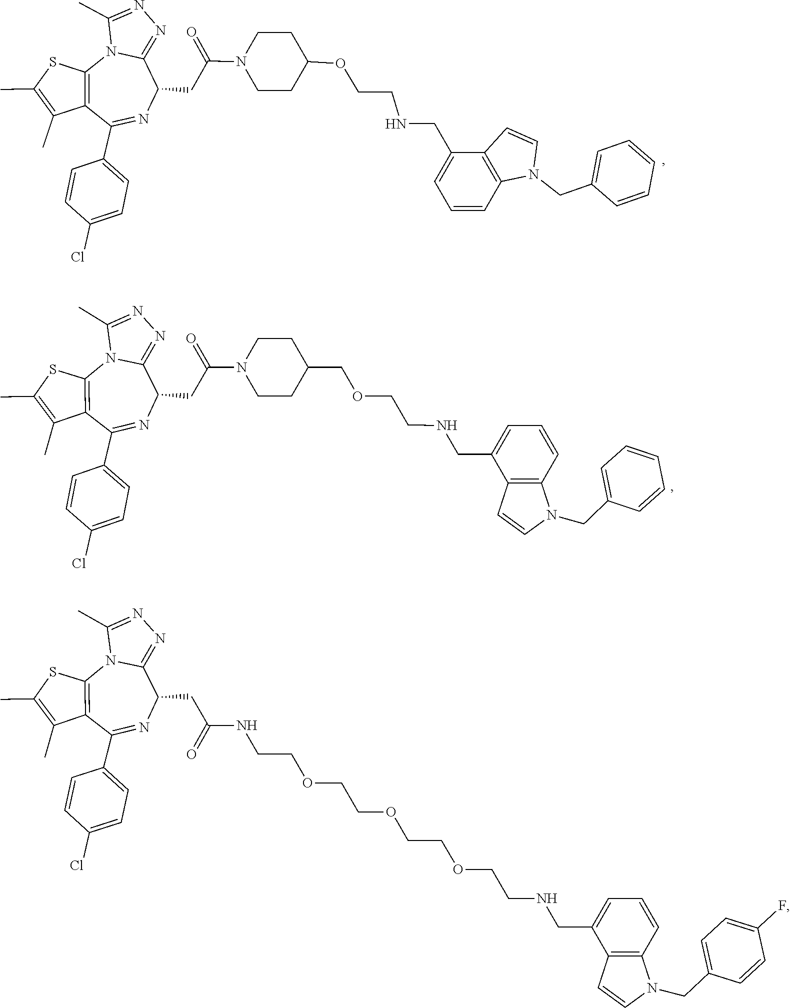 [SMILES: Cc1sc2c(c1C)C(c1ccc(Cl)cc1)=N[C@@H](CC(=O)N1CCC(OCCNCc3cccc4c3ccn4Cc3ccccc3)CC1)c1nnc(C)n1-2 ; Cc1sc2c(c1C)C(c1ccc(Cl)cc1)=N[C@@H](CC(=O)N1CCC(COCCNCc3cccc4c3ccn4Cc3ccccc3)CC1)c1nnc(C)n1-2 ; Cc1sc2c(c1C)C(c1ccc(Cl)cc1)=N[C@@H](CC(=O)NCCOCCOCCOCCNCc1cccc3c1ccn3Cc1ccc(F)cc1)c1nnc(C)n1-2] 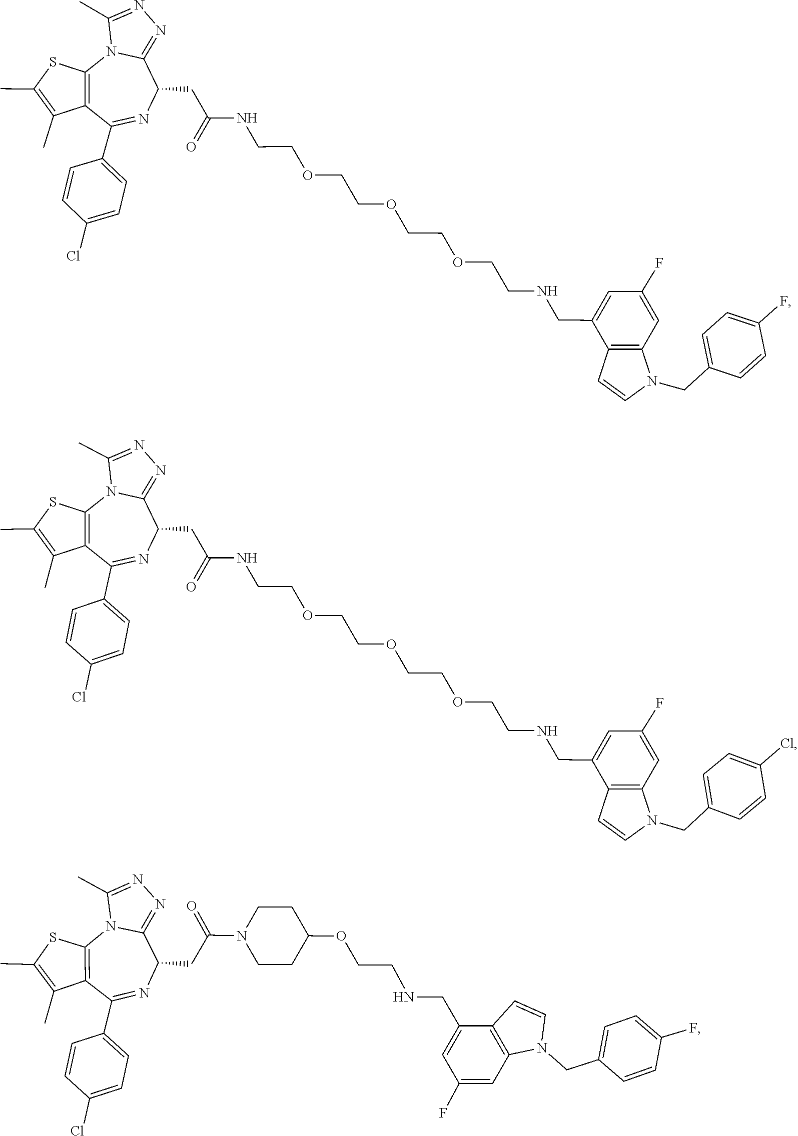 [SMILES: Cc1sc2c(c1C)C(c1ccc(Cl)cc1)=N[C@@H](CC(=O)NCCOCCOCCOCCNCc1cc(F)cc3c1ccn3Cc1ccc(F)cc1)c1nnc(C)n1-2 ; Cc1sc2c(c1C)C(c1ccc(Cl)cc1)=N[C@@H](CC(=O)NCCOCCOCCOCCNCc1cc(F)cc3c1ccn3Cc1ccc(Cl)cc1)c1nnc(C)n1-2 ; Cc1sc2c(c1C)C(c1ccc(Cl)cc1)=N[C@@H](CC(=O)N1CCC(OCCNCc3cc(F)cc4c3ccn4Cc3ccc(F)cc3)CC1)c1nnc(C)n1-2] 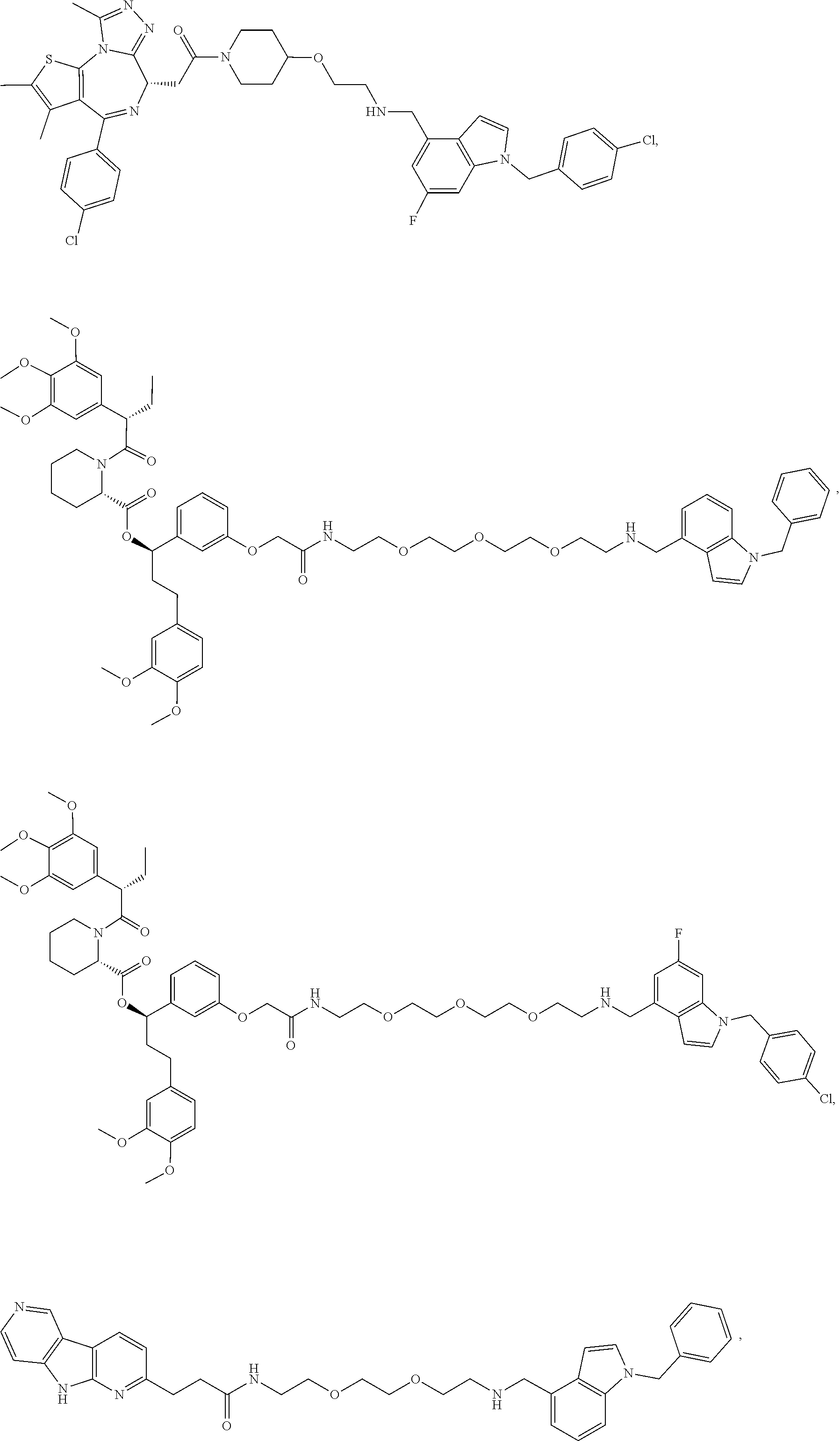 [SMILES: Cc1sc2c(c1C)C(c1ccc(Cl)cc1)=N[C@@H](CC(=O)N1CCC(OCCNCc3cc(F)cc4c3ccn4Cc3ccc(Cl)cc3)CC1)c1nnc(C)n1-2 ; CC[C@H](C(=O)N1CCCC[C@H]1C(=O)O[C@H](CCc1ccc(OC)c(OC)c1)c1cccc(OCC(=O)NCCOCCOCCOCCNCc2cccc3c2ccn3Cc2ccccc2)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCNCc2cc(F)cc3c2ccn3Cc2ccc(Cl)cc2)c1)c1cc(OC)c(OC)c(OC)c1 ; O=C(CCc1ccc2c(n1)[nH]c1ccncc12)NCCOCCOCCNCc1cccc2c1ccn2Cc1ccccc1] 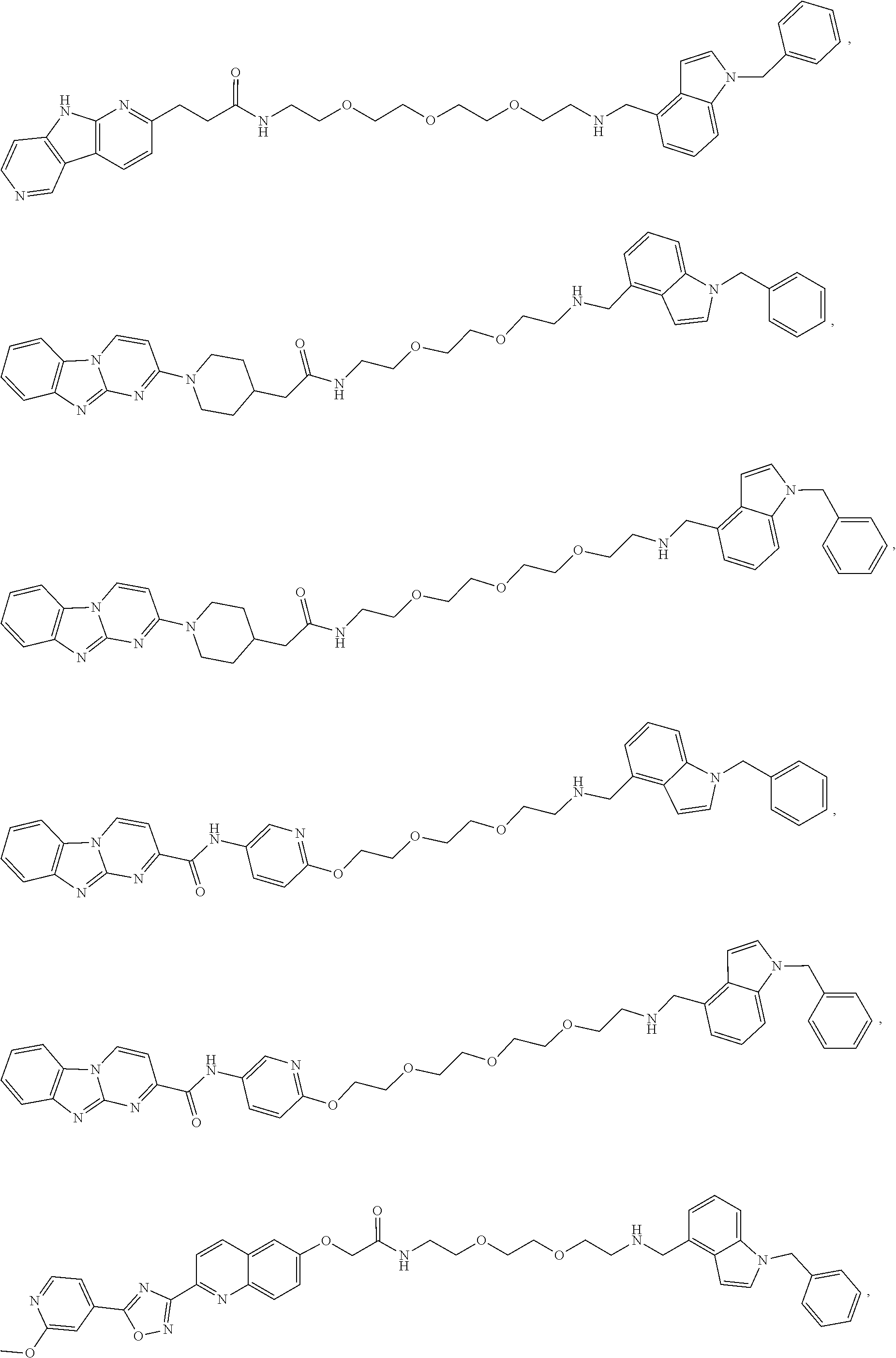 [SMILES: O=C(CCc1ccc2c(n1)[nH]c1ccncc12)NCCOCCOCCOCCNCc1cccc2c1ccn2Cc1ccccc1 ; O=C(CC1CCN(c2ccn3c(n2)nc2ccccc23)CC1)NCCOCCOCCNCc1cccc2c1ccn2Cc1ccccc1 ; O=C(CC1CCN(c2ccn3c(n2)nc2ccccc23)CC1)NCCOCCOCCOCCNCc1cccc2c1ccn2Cc1ccccc1 ; O=C(Nc1ccc(OCCOCCOCCNCc2cccc3c2ccn3Cc2ccccc2)nc1)c1ccn2c(n1)nc1ccccc12 ; O=C(Nc1ccc(OCCOCCOCCOCCNCc2cccc3c2ccn3Cc2ccccc2)nc1)c1ccn2c(n1)nc1ccccc12 ; COc1cc(-c2nc(-c3ccc4cc(OCC(=O)NCCOCCOCCNCc5cccc6c5ccn6Cc5ccccc5)ccc4n3)no2)ccn1] 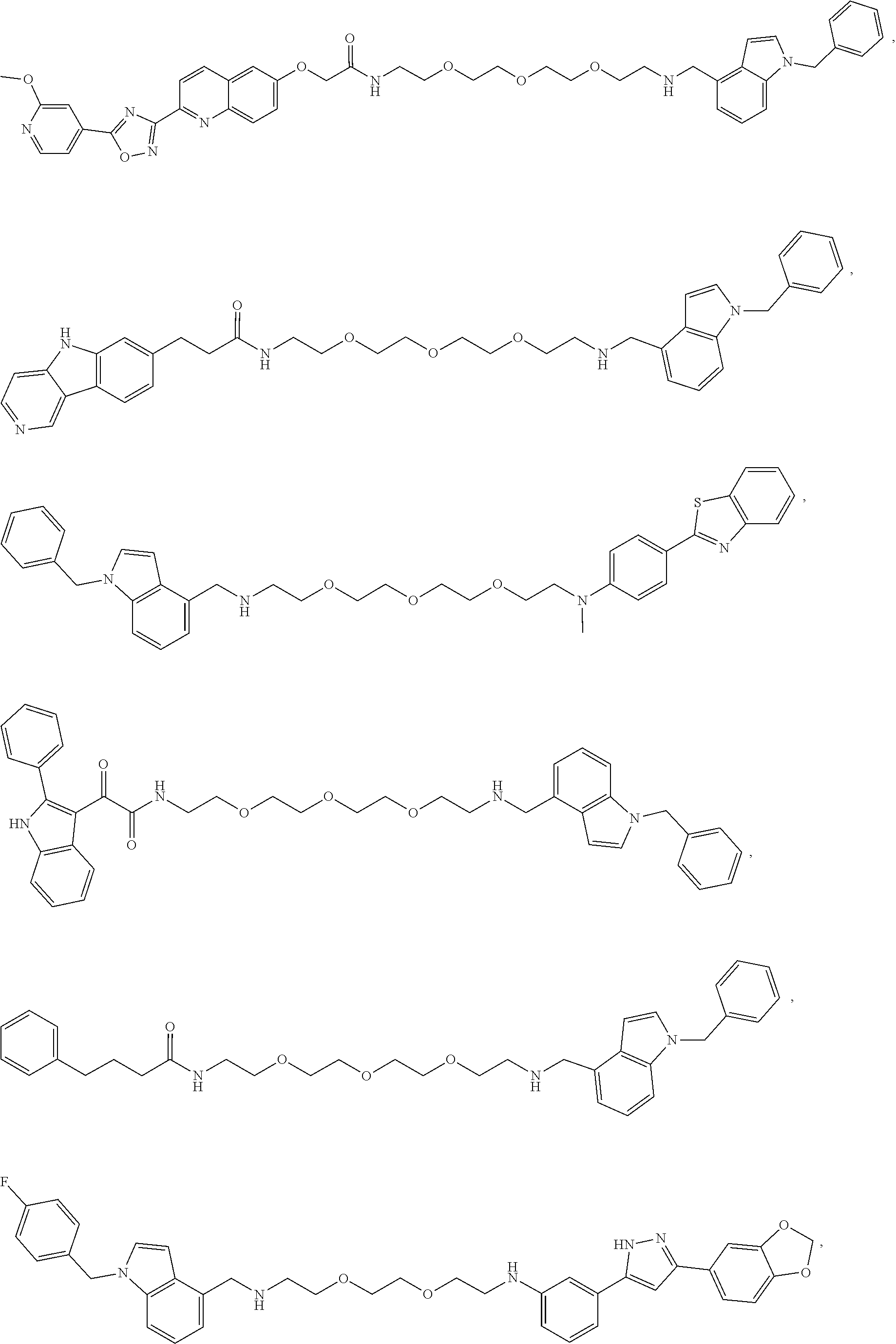 [SMILES: COc1cc(-c2nc(-c3ccc4cc(OCC(=O)NCCOCCOCCOCCNCc5cccc6c5ccn6Cc5ccccc5)ccc4n3)no2)ccn1 ; O=C(CCc1ccc2c(c1)[nH]c1ccncc12)NCCOCCOCCOCCNCc1cccc2c1ccn2Cc1ccccc1 ; CN(CCOCCOCCOCCNCc1cccc2c1ccn2Cc1ccccc1)c1ccc(-c2nc3ccccc3s2)cc1 ; O=C(NCCOCCOCCOCCNCc1cccc2c1ccn2Cc1ccccc1)C(=O)c1c(-c2ccccc2)[nH]c2ccccc12 ; O=C(CCCc1ccccc1)NCCOCCOCCOCCNCc1cccc2c1ccn2Cc1ccccc1 ; Fc1ccc(Cn2ccc3c(CNCCOCCOCCNc4cccc(-c5cc(-c6ccc7c(c6)OCO7)n[nH]5)c4)cccc32)cc1] 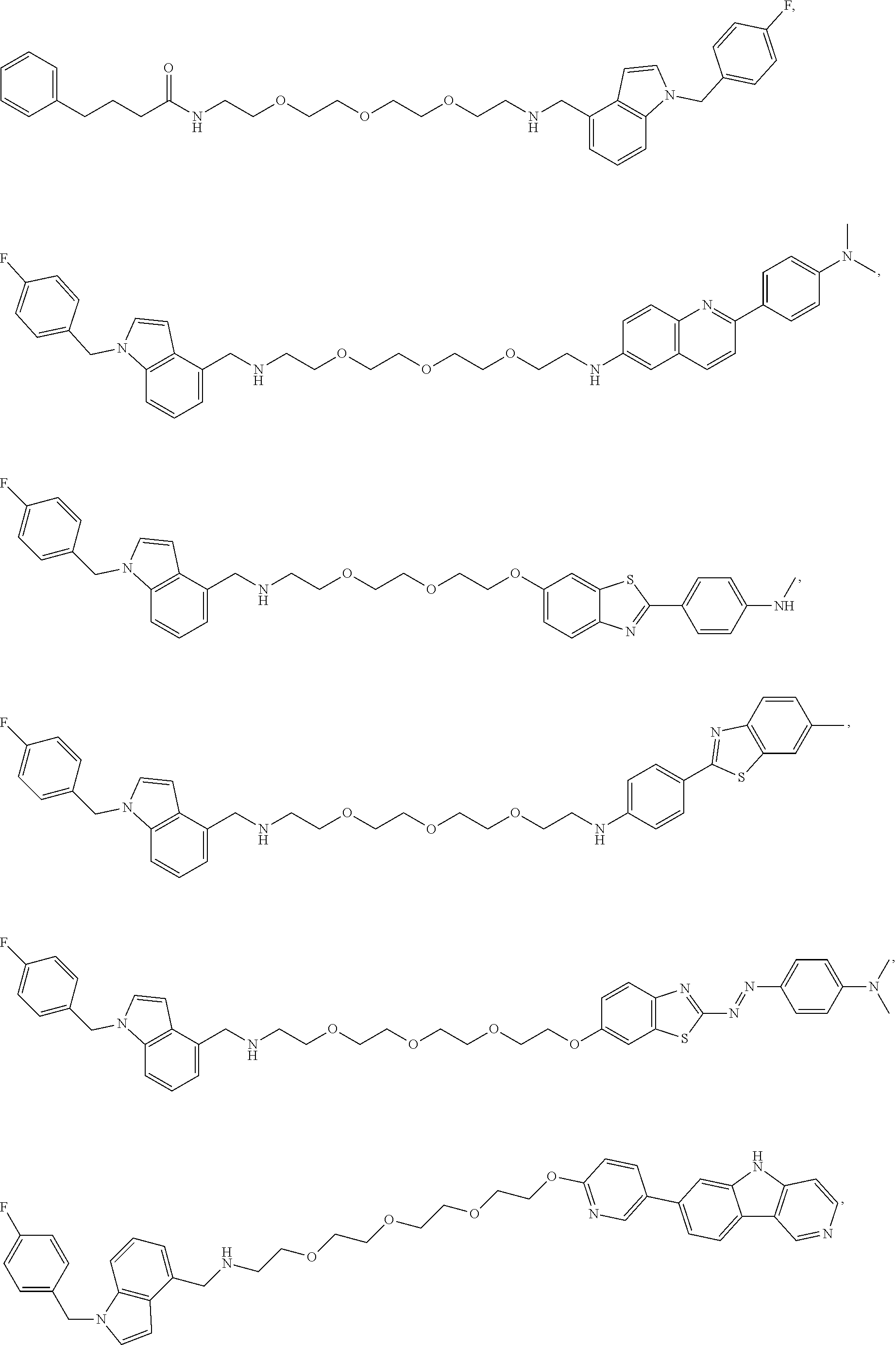 [SMILES: O=C(CCCc1ccccc1)NCCOCCOCCOCCNCc1cccc2c1ccn2Cc1ccc(F)cc1 ; CN(C)c1ccc(-c2ccc3cc(NCCOCCOCCOCCNCc4cccc5c4ccn5Cc4ccc(F)cc4)ccc3n2)cc1 ; CNc1ccc(-c2nc3ccc(OCCOCCOCCNCc4cccc5c4ccn5Cc4ccc(F)cc4)cc3s2)cc1 ; Cc1ccc2nc(-c3ccc(NCCOCCOCCOCCNCc4cccc5c4ccn5Cc4ccc(F)cc4)cc3)sc2c1 ; CN(C)c1ccc(/N=N/c2nc3ccc(OCCOCCOCCOCCNCc4cccc5c4ccn5Cc4ccc(F)cc4)cc3s2)cc1 ; Fc1ccc(Cn2ccc3c(CNCCOCCOCCOCCOc4ccc(-c5ccc6c(c5)[nH]c5ccncc56)cn4)cccc32)cc1] 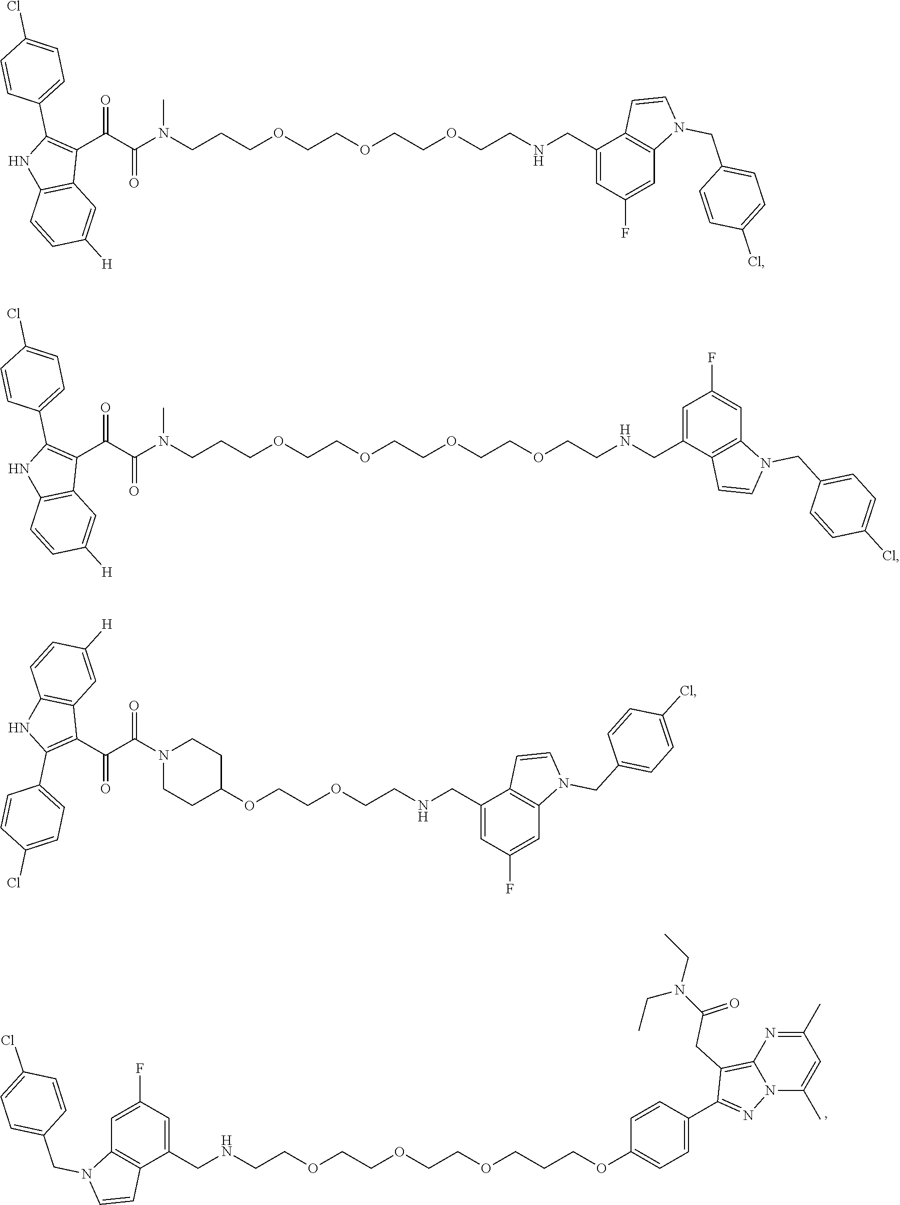 [SMILES: CN(CCCOCCOCCOCCNCc1cc(F)cc2c1ccn2Cc1ccc(Cl)cc1)C(=O)C(=O)c1c(-c2ccc(Cl)cc2)[nH]c2ccccc12 ; CN(CCCOCCOCCOCCOCCNCc1cc(F)cc2c1ccn2Cc1ccc(Cl)cc1)C(=O)C(=O)c1c(-c2ccc(Cl)cc2)[nH]c2ccccc12 ; O=C(C(=O)N1CCC(OCCOCCNCc2cc(F)cc3c2ccn3Cc2ccc(Cl)cc2)CC1)c1c(-c2ccc(Cl)cc2)[nH]c2ccccc12 ; CCN(CC)C(=O)Cc1c(-c2ccc(OCCCOCCOCCOCCNCc3cc(F)cc4c3ccn4Cc3ccc(Cl)cc3)cc2)nn2c(C)cc(C)nc12] 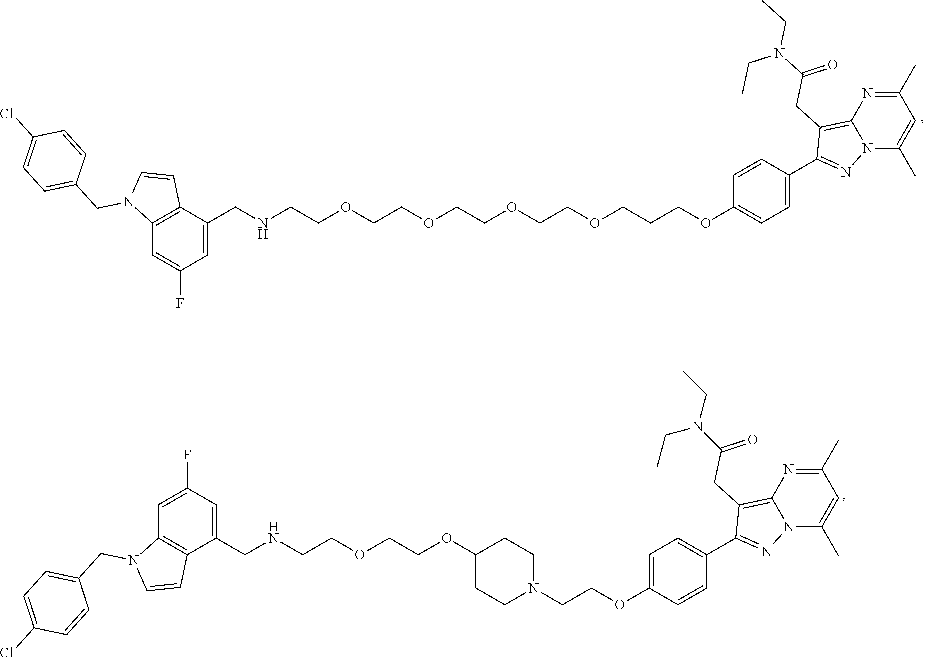 [SMILES: CCN(CC)C(=O)Cc1c(-c2ccc(OCCCOCCOCCOCCOCCNCc3cc(F)cc4c3ccn4Cc3ccc(Cl)cc3)cc2)nn2c(C)cc(C)nc12 ; CCN(CC)C(=O)Cc1c(-c2ccc(OCCN3CCC(OCCOCCNCc4cc(F)cc5c4ccn5Cc4ccc(Cl)cc4)CC3)cc2)nn2c(C)cc(C)nc12]
or a pharmaceutically acceptable salt, a stereoisomer, or deuterated form thereof.

91.-97. (canceled)

98. A method of degrading a target protein in a subject in need thereof, comprising administering a compound of claim 1.

99.-101. (canceled)
